(19) United States
(12) Patent Application Publication
(10) Pub. No.: US 2014/0086881 A1
(43) Pub. Date: Mar. 27, 2014

(54) GOOD HEALTHY CELLS FOUND IN PROTEINS, THEIR APPLICATIONS, AND PROCESS OF MAKING A MEDIUM TO HARVEST THE CELLS

(71) Applicant: Kieu Hoang, Agoura Hills, CA (US)

(72) Inventor: Kieu Hoang, Agoura Hills, CA (US)

(21) Appl. No.: 13/756,463

(22) Filed: Jan. 31, 2013

Related U.S. Application Data
(60) Provisional application No. 61/593,164, filed on Jan. 31, 2012.
(60) Provisional application No. 61/593,183, filed on Jan. 31, 2012.
(60) Provisional application No. 61/593,196, filed on Jan. 31, 2012.
(60) Provisional application No. 61/648,281, filed on May 17, 2012.
(60) Provisional application No. 61/692,273, filed on Aug. 23, 2012.
(60) Provisional application No. 61/710,930, filed on Oct. 8, 2012.

(51) Int. Cl. A61K 36/899

(52) U.S. Cl. CPC A61K 36/899
USPC 424/93.7; 435/375; 435/325; 435/366; 435/410; 435/412; 435/411; 435/349; 530/350

(57) ABSTRACT

GOOD HEALTHY DRAGON, SNAKE, DIFFERENT SIZE DOUBLE RINGS, LIGHTNING, SQUARE PIXEL, BEAMING RAYS, RECONSTRUCTION BACKGROUND, FACET, CRATER, YELLOW, LEER CELLS were found in New Proteins (among them 27 new ones and their sequences (Under a different patent application) or in the existing discovered proteins and their applications. The process of making the medium derived from any source to harvest any cell—named KH cells—KH cells are good healthy cells in which the RNA synthesizes good proteins that: 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals to increase the protein yield for the application of the cell expression of human healthcare, animal healthcare and plant healthcare including fertilizer and maximize production of medicine, food, fruit, juice, meat, seafood and plants.

### SUMMARY OF THE INVENTION

GOOD HEALTHY DRAGON, SNAKE, DIFFERENT SIZE DOUBLE RINGS, LIGHTNING, SQUARE PIXEL, BEAMING RAYS, RECONSTRUCTION BACKGROUND, FACET, CRATER, YELLOW, LEER CELLS were found in New Proteins (among them 27 new ones and their sequences (Under a different patent application) or in the existing discovered proteins and their applications. In addition, the process of making the medium derived from any source to harvest any cell—named KH cells—KH cells are good healthy cells in which the RNA synthesizes good proteins that: 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals to increase the protein yield for the application of the cell expression of human healthcare, animal healthcare and plant healthcare including fertilizer and maximize production of medicine, food, fruit, juice, meat, seafood and plants.

### INVENTOR: Kieu Hoang

### 30423 Canwood Street, #120 Agoura Hills Calif. 91301

### BACKGROUND

Some says in a human body, there are between 10 trillions, 50 trillions to 70 trillions of cells which are essential in making a person life healthy with GOOD HEALTHY CELLS.

BAD DAMAGED OR SICK CELLS make a person SICK, and the Death of Cells will end a person life.

So far about 210 human cells have been discovered and identified.

27 more New proteins and their sequence have been discovered by Inventor and therefore MORE GOOD HEALTHY CELLS in these proteins as well as in the found proteins have been discovered.

In Vietnam history and culture, the SNAKE goes together with DRAGON, an animal Which is not real among the 11 real animals that Buddha has allowed for all animals to compete in order to select 12 animals to control and to govern man kind and provide horoscope for those who were born in the yea there is no rs of these animals including: 1: Mice 2: Buffalo 3:Tiger 4: Cat 5: DRAGON 6 SNAKE 7:Horse 8:Goat 9:Monkey 10:Chicken 11:Dog 12: Pig

DRAGON is not a REAL ANIMAL so why it can be chosen by Buddha.

Dragon lives in water, fly in sky why did it end up in Number 5 in this competition it is quite strange for Dragon as it can swim very quickly quicker than Mice, Buffalo, and CAT. This is the first strange point.

The second strange point is that the DRAGON is NOT A REAL ANIMAL. Why our ancestors in the East as well as in the WEST from the stone ages, with no means of communication, but all thought and had the imagination about an ANIMAL which is called DRAGON is the same with its MYSTERY.

The Origin of Dragon: Mankind from the EAST to the WEST have tried to analyze explain the origin, the image and symbol of the DRAGON as follows:

### 1. DRAGON of the EAST:

Every country has recognized that DRAGON is not REAL, an imagination French language in the beginning 13^{th} centuries (much later than China and Vietnam) called Dragon as DRAGE from Latin language: Draconem

And it also has the meaning: A BIG SNAKE. Egyptian language called DRAKON, which means SNAKE or a GIANT WATER SNAKE. English language: DRAGON came from DRA'KO'N of Greece which also means a very long Water Snake.

### 2. DRAGON of the WEST:

In China and its neighboring countries, DRAGON is one of the Four Long, Lan, Quy, Phung (Vietnamese name of these four animals),

1. Dragon (Long), Chinese Lions (Lan) that one can easily see in Las Vegas at the entrance of hotels, Turtle (Quy) which is ONLY REAL ANIMAL among the four. The oldest turtle with thousand of years is still living in Hoan Kiem Lake in Hanoi. Vietnam. The fourth is a very rare big bird, which is only in imagination.

At the end of 1987, they have discovered a Dragon made of ceramics in Hanam province from where 1^{st} generation ancestors of Inventor started in the North of Vietnam.

Archeologists have evaluated this dragon was made 6000 years ago.

SONS OF DRAGON and NIECES of Beautiful fairy. (CON RONG CHAU TIEN (Vietnamese language.)

The history and culture of Vietnam is related to the DRAGON since 2878 B.C So Vietnam has been established and found nearly 5000 years of history.

Being a Vietnamese or Vietnamese origins, Our father named is LAC and our mother named is AU.

Father LAC is LAC LONG QUAN, aka SUNG LAM, a top ranking leading farmer, and king of the South.

Thanks to his talent and his ability to govern to bring peace prosperity together with his kindness and generosity to his people everywhere living in A Paradise on Earth, therefore people consider him as A DRAGON.

At that time in a neighboring country, a very beautiful charming gentle virtue

Lady and everybody in this neighboring country consider her as a Beautiful Fairy. Lac long Quan has wedded her. They lived together for one year and she gave birth to one bag of 100 eggs. 7 days later, 50 boys were born from 50 eggs and

50 girls from 50 eggs. One day Lac Long Quan told his wife:

I am the DRAGON you are BEAUTIFUL FAIRY. Now it is the time to part as FIRE is against the WATER and it is difficult to live together in unity for a long time.

Now I will take 50 sons of ours and will go up to the lowland coastal areas and you will take 50 daughters up to the mountains in order to govern all regions of this country.

Regardless up in the mountain or in the coastal areas if there is event or anything that we should let each other know and we should never part.

Bidding good-bye to mother Au Co, Lac Long Quan took 50 boys to the South Lowland coastal areas.

His eldest son named HUNG VUONG and has been heir to the throne to govern the VIET RACE and established HONG BANG dynasty from this point on since 2878B. R and Vietnam was founded nearly 5000 years ago.

So the name of DRAGON has been mentioned nearly 5000 years ago from the EAST to the WEST, so we must assume that our ancestors had for some how knew the existence of this kind of creature even in the imagination

Thousand years ago, People in the east can even built Great wall with bare hand and people in West has built ROME and GREECE, and in Middle East has built Pyramids with also Bare Hands and with their intelligence.

We have seen the mummies in Egypt but all these mummies were wrapped up and the corpse cannot be seen in FULL but in HeNan (Province) in ChangSha city where our Shumen plasma center (One of the three plasma centers from where Dragon Cell originated) another corpse of the princess has been displayed in the Province Museum, the coffin contained this corpse which was buried around 2500 years ago and the corpse is in good shape without using frozen technology

The technology to keep the corpse of this princess as well as all jewelries, silks look new and not damaged.

With these advanced technologies that we could not have it today it is possible That our ancestor's scientists may have discovered this GENE of a human being long time ago Based upon Vietnamese history culture theory DRAGON is not AN ANIMAL DRAGON is the leading FARMER LAC LONG QUAN who ruled the beginning period before King HUNG VUONG (His eldest son) was a HUMAN BEING nearly 5000 years ago.

Nearly 5000 years later, the theory of Vietnamese people about Dragon as a HUMAN BEING is proven by the INVENTOR who is also A Vietnamese American who has discovered GOOD HEALTHY DRAGON CELL from Human Plasma

### DESCRIPTIONS OF THE DRAWING FIGURES

FIG. 1.1 through 1.5—Images taken from different wells at different positions from Cry pool plasma lot#20110810-4B consisting of approximately 5,000 liters of plasma from 3 plasma centers from Quang Xi (Quan Xi has the oldest person that live up to 129 years old in China) and Hunan Province were used to culture. After centrifugation the paste and the supernatant were used to culture on Aug. 20, 2011 and this plates containing the cells, which still live and grow until Jan. 12, 2012 when we wrote this document for patent, 145 days have passed. This is amazing finding as most scientist conclude that the cell will live only for 7 days in a culture medium.

FIGS. 2.1 and 3.1—FIG. 1.1 through 1.5—Images taken from different wells at different positions at later dates from Cry pool plasma lot#20110810-4B consisting of approximately 5,000 liters of plasma from 3 plasma centers from Quang Xi (Quan Xi has the oldest person that live up to 129 years old in China) and Hunan Province were used to culture. After centrifugation the paste and the supernatant were used to culture on Aug. 20, 2011 and this plates containing the cells, which still live and grow until Jan. 12, 2012 when we wrote this document for patent, 145 days have passed. This is amazing finding as most scientist conclude that the cell will live only for 7 days in a culture medium.

FIG. 4.1 through 4.36—Beginning from day 1 until day 31^{st} when being asked by the inventor the progress of the cell culture, the scientist in charge of the cell culture report that there are not cells only the fragment of the dead cell. As she used dye to see if the cell is alive or dead she concluded that there were all dead segment of the cell. At this time is when the inventor got heavily involved to monitor the growth of the cells from day 31. The cells begin to grow with different shapes like lining, double ring, square cell, snake cell, dragon cell, etc. In order to prove that they are living cell then the inventor ordered the scientist to use the pipette to stir at the bottom of the plate to destroy everything in that well. And transfer half of the medium into two more plates. (Plate #2 and #3)

FIG. 4.37 through 4.90—Images captured from live video taken from the original plate after mix showing moving cells. In these images we can observe different type of cells in shape and size move through the well.

FIG. 5.1 through 5.40—Images captured from microscope taken from Plate#2, which consists of 12 wells and a blank control well. On this plate in well#5 we discovered the appearance of the Dragon cell on Oct. 20, 2011.

FIG. 5.41 through 5.47—Images captured from microscope taken from Plate#2 of different type of non-moving cells. These cells may have moved in the wells but we have no record of this.

FIG. 5.48—Original plate #1 from well number 5 from where the dragon originated. No dragon in this well. FIG. 5.49 through 5.51—Images captured of the GOOD HEALTHY Dragon cell during different dates, from when it originated till the Jan. 10, 2012.

FIG. 5.52 through 5.130—Images captured from live video of Plate #2 Well #5 of the GOOD HEALTHY Dragon cell. Images reflect movement of this cell during a 12 minute long video. The cell moves up and down repeatedly and also blurs in and out of the video.

FIG. 5B.1—Transfer Plate number 3 of the medium well number 5 into the breast and lung cancer cell. The medium containing good healthy cell vs Breast Cancer Cell. The good healthy cell has attacked the cancer cell and transformed it into a good healthy cell.

FIG. 5B.2—Third transfer of the medium from the Dragon Well Plate number 2. After 90 days still no Dragon Cell appeared only cluster of different cells including new found and already discovered ones.

FIG. 5B3—Fifth transfer from the Dragon Well #5 medium 400 micro liters for another CRO lab to identify the cells.

FIG. 6.1 through 6.16—Transfer Plate 3 Breast & Lung Cancer. In order to know certain degree of the killing effect of these cells we used the medium to put in the breast cancer cell. From day 1, Sep. 30, 2011 to 49 days and continue on until 104 days (Jan. 12, 2012) and these cells after killing the cancer cells they may have transformed into good healthy cell.

FIG. 6.17 through 6.32—Images taken from live video of plate #3 where cancer cells were introduced. It was observed different type of moving cells changing the background.

FIG. 7.1 through 7.4—Images taken form transfer plate #4 where we put our AFOD (7.5% with 12.5% stabilizer) product vs breast cancer cells.

FIG. 7.5 through 7.8—Images taken form transfer plate #4 where we put our AFCC (6 kg-600 kg-60 kg) product vs breast cancer cells.

FIG. 7.9 through 7.12—Images taken form transfer plate #4 where we put our AFCC (from column last elution) product vs breast cancer cells.

FIG. 8.1 through 8.4—Images taken from transfer plate #5 where we put our AFCC KH product vs breast cancer cells.

FIG. 9.1—Images taken from plate #6. Tissue from mice #3-7 treated with AFOD & AFCC and the type of cell it grew. This mice tumor has been self-detached from the body.

FIG. 9.2 through 9.5—Images taken from plate #6. Tissue from different mice treated with AFOD & AFCC in comparison to mice treated with Docetaxcel against breast cancer and the type of cell it grew.

FIG. 9.6 through 9.7—Images taken from plate #6. Tissue from different mice treated with AFOD in comparison to mice treated with Docetaxcel against lung cancer and the type of cell it grew.

FIGS. 10.1 and 10.2—Images taken from plate #1 after third transfer. In order to identify the type of cell we have grown in the main 6 plates from the second transfer, we took 400 micro liters of medium and transferred this medium for a third time into 12 wells. We observed different type of cells in these 12 wells, including GOOD HEALTHY Snake cell.

FIGS. 10.3 and 10.4—Images taken from plate #2 after third transfer. In order to identify the type of cell we have grown in the main 6 plates from where we have taken 400 micro liters of medium and transferred this medium third time. We did not discover a GOOD HEALTHY Dragon cell after this third transfer but we did find 3 GOOD HEALTHY Snake cells and GOOD HEALTHY double ring cells.

FIGS. 10.5 and 10.6—Images taken from plate #3 after the third transfer. In order to identify the type of cell we have grown in the main 6 plates from where we have taken 400 micro liters of medium and transferred this medium third time. This plate contains medium with cancer cells.

FIGS. 10.7 and 10.8—Images taken from plate #4 after the third transfer. In order to identify the type of cell we have grown in the main 6 plates from where we have taken 400 micro liters of medium and transferred this medium third time. This plate contains our products AFCC and AFOD vs breast cancer cells.

FIGS. 10.9 and 10.10—Images taken from plate #5 after the third transfer. In order to identify the type of cell we have grown in the main 6 plates from where we have taken 400 micro liters of medium and transferred this medium third time. This plate contains our products AFCC and AFOD vs breast cancer cells.

FIGS. 10.11 and 10.12—Images taken from plate #6 after the third transfer. In order to identify the type of cell we have grown in the main 6 plates from where we have taken 400 micro liters of medium and transferred this medium third time. This plate contains the culture of the mice tissue with breast and lung cancer vs our AFOD and Docetaxcel.

FIG. 11.1 through 11.58—Images taken from live video of the 6 plates after the third transfer. In these pictures we have identified many different type of cells, just like GOOD HEALTHY Snake cell mainly from plate #1 and plate #5 along with GOOD HEALTHY Double ring cell.

FIG. 11a.1 through 11a.5—Images taken plate culture of living mice tissue treated with our product AFOD and AFCC.

FIG. 12.1 through 12.14—Images taken from plate culture of GOOD HEALTHY cell from AFOD 10% product. In these pictures we found the moving living cells as well, mainly double ring and background reconstruction cells.

FIG. 13.1 through 13.12—Images taken from plate culture of GOOD HEALTHY cells from AFCC product. In these pictures we found moving living cells as well,

FIG. 14.1 through 14.16—Images taken from plate culture of lung cancer cells.

FIG. 15.1 through 15.14—Images taken from live video of plate culture of CHO cells. CHO cells move slowly and we do have background cells. There was neither double ring cell nor lining cell like we observed in AFOD & AFCC

FIG. 16.33 through 16.68—Images taken from live video of culture plate #3 containing our AFOD product vs lung cancer cells. We observed a lot of activity in moving cells, but mainly from the GOOD HEALTHY Double ring cells either single or moving in groups.

FIG. 17.1 through 17.20—Images taken from live video of plate culture #3 containing our product AFCC vs lung cancer cells. We observed a lot of activity in moving cells. Both from GOOD HEALTHY Double ring cells and also other type of cells.

FIG. 18.1 through 18.16—Images taken from live video of plate culture containing our product AFOD vs CHO cell. We observed a lot of living moving Double ring cells.

FIG. 19.1 through 19.28-Images taken from live video of plate culture containing lung cancer cells vs our product AFOD. We observed a lot of living moving cells. In FIG. 19.4 through 19.6 we observed GOOD HEALTHY Lighting cell moving across the screen. We also observed GOOD HEALTHY Double ring cells in different shapes and moving at different speeds. FIG. 19.20 through 19.24 we observed a single double ring cell move upward in the screen leaving a trail behind.

FIG. 20.1 through 20.6—Images taken from live video of plate culture containing lung cancer cells vs our product AFCC. We observed mainly living moving GOOD HEALTHY Double ring cells.

FIG. 20.7—Images taken from plate culture containing living GOOD HEALTHY cells from our products AFOD and AFCC.

FIG. 20.8—Image taken from plate culture containing lung cancer cells. These were the cells that we used to mix into our products AFOD and AFCC culture plates.

FIG. 20.9 Images taken from plate culture containing our products AFOD and AFCC after we mixed the lung cancer cells showed from picture 20.8. We observed that both concentrations of our products transformed the lung cancer cells into good healthy cells. We also observed more transformation in the higher concentrations of our products AFOD and AFCC.

FIG. 21.1 through 21.21—Picture taken from culture plates containing GOOD HEALTHY Snake cells showing the DNA of the cell.

FIG. 22a.1—Picture taken from CRO lab during mice pilot studies ensuring the good practices of animal care during the investigations.

FIG. 22.1—Chart recording the growth of the tumor volume on nude mice #3-7 vs Docetaxcel and vehicle control. On date 87 of introducing the tumor, the tumor itself detached from the body of the mice.

FIGS. 22.2 and 22.3—Pictures of mice 3-7 documenting the growth of the tumor until the 87^{th} day on Oct. 19, 2011 when the tumor detached from the body.

FIG. 22.4—Chart recording the tumor measurements from start till Jan. 19, 2012. For mice #1-5, 3-7 and 4-6 the values are 0 because on all three mice the tumor popped out.

FIG. 22.5—Pictures of mice #3-7 66 days after re-implantation.

FIG. 22.6—Chart recording the growth of the tumor volume on nude mice #4-6 vs Docetaxcel and vehicle control. On date 39 of introducing the tumor, the tumor itself detached from the body of the mice.

FIGS. 22.7 and 22.8—Pictures of mice 4-6 documenting the growth of the tumor until the 39^{th} day on Aug. 30, 2011 when the tumor detached from the body.

FIG. 22.9—Pictures of mice #4-6, 59 days after re-implantation.

FIG. 22.10—Picture of mice #4-6. Picture taken after treatment was stopped. We discovered that this particular mice, which is a nude mice and cannot grow hair, had grown hair in the top of the head.

FIG. 23.1—Picture of mice #3-7 on October 18, a day before the tumor detached.

FIG. 23.2 through 23.5—Pictures of cultured tumor from mice #3-7 which originally detached by itself from the body of the mice.

FIG. 23.6—Picture of re-culture tumor from mice #3-7 which originally detached by itself from the body of the mice. Tissue re-cultured on Jan. 26, 2012.

FIGS. 23.7 and 23.8—Picture taken from re-cultured tumor of Mice #3-7. We observed living moving cells, including GOOD HEALTHY Beaming cell and GOOD HEALTHY Snake cell.

FIGS. 23.9 and 23.10—Picture taken from live video of re-cultured tumor of Mice #3-7, where we observed movement of living GOOD HEALTHY Snake cell. This is the first time we have seen any GOOD HEALTHY Snake cell moving.

FIG. 23.11—Picture taken from culture media of lot# HA20020308A0 of human Albumin collected in 2002 still showing living cells.

FIG. 23.12—Picture taken from culture media of lot# HA200701A001 of human Albumin collected in 2007 still showing living cells.

FIG. 23.13—Picture taken from culture media of lot#20031211A0 of human Immunoglobulin collected in 2003 still showing living cells.

FIG. 23.14—Picture taken from culture media of lot#200701G003 of human Immunoglobulin collected in 2007 still showing living cells.

FIGS. 23.15 and 23.16—Pictures taken form live video of living cells in Immunoglobulin from lot collected in 2007. We observed mainly GOOD HEALTHY Double ring cells and background cells.

FIGS. 23.17 and 23.18—Pictures taken from live video of living moving cells in Human Albumin from lot collected in 2007. We mainly observed GOOD HEALTHY Double ring cells.

FIG. 23.19—Pictures taken from culture plate of plasma collected in 2001 displaying different types of living cells.

FIG. 23.20—Pictures taken from culture plate of Fraction IV collected in 2001 showing different types of living cells.

FIGS. 24.1 and 24.2—Chart and picture of the composition of our Product AFCC containing a sequence of 26 proteins.

FIGS. 25.1 and 25.2—Chart and picture of the composition of our Product AFOD containing a sequence of 15 proteins.

FIG. 26.1—Sample of 10 year old Human Albumin.

FIG. 26.2—Sample of 10 years old of Human Immunoglobulin.

FIG. 26.3—Photos of mouse 3-7 showing tumor pop-out.

FIG. 26.4—Cultured plate of tumor cells from mouse 3-7.

FIG. 26.5—Picture of Pork fat medium.

FIG. 26.6—Picture of Pork fat medium with cell count.

FIG. 26.7—Picture of Chicken fat medium.

FIG. 26.8—Picture of Chicken fat medium with cell count.

FIG. 26.9—Picture of Beef fat medium.

FIG. 26.10—Picture of Beef fat medium with cell count.

FIG. 26.11—Mangos teen

FIG. 26.12—Cucumber

FIG. 26.13—Lettuce

FIG. 26.13B—CHO cell

FIG. 26.14—Dose-dependent curves (CC50 values)

FIG. 26.15—Dose-dependent curves (CC50 values)

FIG. 26.16—Dose-dependent curves (EC50 values)z

FIG. 26.17—CC50 and EC50 Summary of the human plasma derived proteins

FIG. 26.18—Anti-tumor efficacy of high concentrated fibrinogen enriched a1 at thrombin and Afod in PDX model LU-01-0032

FIG. 26.19—Dose-dependent curves (EC50 values)

FIG. 26.20—Dose-dependent curves (EC50 values)

FIG. 26.21—CC50 and EC50 Summary of the human plasma derived proteins

FIG. 26.22—Photographs of tumors dissected from abdominal cavity of each group

FIG. 26.23—Ratios of mice with palpable tumors observed in each group

FIG. 26.24—Relative change of body weight (%) of different groups

FIG. 27.1—Sample of KH101 (non-sticky rice)

FIG. 27.2—Sample of KH101 (non-sticky rice) with cell count

FIG. 27.3—Sample of KH102 (Urine)

FIG. 27.4—Sample of KH102 (Urine) with cell count

FIG. 27.5—Sample of KH103 (Soybean)

FIG. 27.6—Sample of KH103 (Soybean) with cell count

FIG. 27.7—Sample of KH104 (Orange Juice)

FIG. 27.8—Sample of KH104 (Orange Juice) with cell count

FIG. 27.9—Sample of KH105 (Grape Juice)

FIG. 27.10—Sample of KH105 (Grape juice) with cell count

FIG. 27.11—Sample of KH106 (Apple juice)

FIG. 27.12—Sample of KH106 (Apple juice) with cell count

FIG. 27.13—Sample of KH107 (Sticky rice)

FIG. 27.14—Sample of KH107 (Sticky rice) with cell count

FIG. 27.15—Sample of KH108 (Water for Injection)

FIG. 27.16—Sample of KH108 (Water for Injection) with cell count

FIG. 27.17—Sample of KH109 (White wine)

FIG. 27.18—Sample of KH109 (White wine) with cell count

FIG. 27.19—Sample of KH110 (red wine)

FIG. 27.20—Sample of KH110 (red wine) with cell count

FIG. 27.21—Sample of KH111 (green bean)

FIG. 27.22—Sample of KH111 (green bean) with cell count

FIG. 27.23—Sample of KH112 (Oat)

FIG. 27.24—Sample of KH112 (Oat) with cell count

FIG. 27.25—Sample of KH113 (Chestnut)

FIG. 27.26—Sample of KH113 (Chestnut) with cell count

FIG. 27.27—Sample of KH114 (Dorian)

FIG. 28—Sample of KH114 (Dorian) with cell count

FIG. 29—Sample of KH115 (Raspberry)

FIG. 30—Sample of KH115 (Raspberry) with cell count

FIG. 31—Sample of KH116 (Pear)

FIG. 32—Sample of KH116 (Pear) with cell count

FIG. 33—Sample of KH117 (Jack fruit)

FIG. 34—Sample of KH117 (Jack fruit) with cell count

FIG. 35—Sample of KH118 (water apple)

FIG. 36—Sample of KH118 (Water apple) with cell count

FIG. 37—Sample of KH119 (Mangosteen)

FIG. 38—Sample of KH119 (Mangosteen) with cell count

FIG. 39—Sample of KH120 (Lettuce)

FIG. 40—Sample of KH120 (Lettuce) with cell count

FIG. 41—Sample of KH121 (Corn)

FIG. 42—Sample of KH121 (Corn) with cell count

FIG. 43—Sample of KH122 (Sweet Potato)

FIG. 44—Sample of KH122 (sweet potato) with cell count

FIG. 45—Sample of KH123 (Cucumber)

FIG. 46—Sample of KH123 (Cucumber) with cell count

FIG. 47—Sample of KH124 (Tomato)

FIG. 48—Sample of KH124 (Tomato) with cell count

FIG. 49—Sample of KH125 (Dragon Fruit)

FIG. 50—Sample of KH125 (Dragon Fruit) with cell count

FIG. 51—Sample of KH126 (Water Melon)

FIG. 52—Sample of KH126 (Water Melon) with cell count

FIG. 53—Sample of KH127 (Lychee)

FIG. 54—Sample of KH127 (Lychee) with cell count

FIG. 55—Sample of KH128 (Yellow Melon)

FIG. 56—Sample of KH128 (Yellow Melon) with cell count

FIG. 57—Sample of KH129 (Pineapple)

FIG. 58—Sample of KH129 (Pineapple) with cell count

FIG. 59—Sample of KH130 (Coconut Juice)

FIG. 60—Sample of KH130 (Coconut Juice) with cell count

FIG. 61—Sample of KH131 (Mint)

FIG. 62—Sample of KH131(Mint) with cell count

FIG. 63—Sample of KH132 (Hot Pepper)

FIG. 64—Sample of KH132 (Hot Pepper) with cell count

FIG. 65—Sample of KH133 (Black Pepper)

FIG. 66—Sample of KH133 (Black Pepper) with cell count

FIG. 67—Sample of KH134 (Carrot)

FIG. 68—Sample of KH134 (Carrot) with cell count

FIG. 68.1—Sample of KH135 (Banana)

FIG. 68.2—Sample of KH135 (Banana)

FIG. 68.3—Sample of KH136 (Big Banana)

FIG. 68.4—Sample of KH136 (Big Banana)

FIG. 68.5—Sample of KH137 (Small Banana)

FIG. 68.6—Sample of KH137 (Small Banana)

FIG. 68.7—Sample of KH138 (Star Fruit)

FIG. 68.8—Sample of KH138 (Star Fruit)

FIG. 68.9—Sample of KH139 (Pomegranate)

FIG. 68.10—Sample of KH139 (Pomegranate)

FIG. 68.11—Sample of KH140 (Plum)

FIG. 68.12—Sample of KH140 (Plum)

FIG. 68.13—Sample of KH141 (Mango)

FIG. 68.14—Sample of KH141 (Mango)

FIG. 68.15—Sample of KH142 (Green hot pepper)

FIG. 68.16—Sample of KH142 (Green hot pepper)

FIG. 68.17—Sample of KH143 (Red sweet pepper)

FIG. 68.18—Sample of KH143 (Red sweet pepper)

FIG. 68.19—Sample of KH144 (Green sweet pepper)

FIG. 68.20—Sample of KH144 (Green sweet pepper)

FIG. 68.21—Sample of KH145 (Daisy flower)

FIG. 68.22—Sample of KH145 (Daisy flower)

FIG. 68.23—Sample of KH146 (Puer Tea)

FIG. 68.24—Sample of KH146 (Puer Tea)

FIG. 68.25—Sample of KH147 (Walnut)

FIG. 68.26—Sample of KH147 (Walnut)

FIG. 68.27—Sample of KH148 (White bread)

FIG. 68.28—Sample of KH148 (White bread)

FIG. 68.29—Sample of KH149 (Brown bread)

FIG. 68.30—Sample of KH149 (Brown bread)

FIG. 68.31—Sample of KH150 (Garlic)

FIG. 68.32—Sample of KH150 (Garlic)

FIG. 68.33—Sample of KH151 (Ginger)

FIG. 68.34—Sample of KH151 (Ginger)

FIG. 68.35—Sample of KH152 (Persimmon)

FIG. 68.36—Sample of KH152 (Persimmon)

FIG. 68.37—Sample of KH153 (Papaya)

FIG. 68.38—Sample of KH153 (Papaya)

FIG. 68.39—Sample of KH154 (Broccoli)

FIG. 68.40—Sample of KH154 (Broccoli)

FIG. 68.41—Sample of KH155 (Onion)

FIG. 68.42—Sample of KH155 (Onion0

FIG. 68.43—Sample of KH156 (Pumpkin)

FIG. 68.44—Sample of KH156 (Pumpkin)

FIG. 68.45—Sample of KH157 (Wax Gourd)

FIG. 68.46—Sample of KH157 (Wax Gourd)

FIG. 68.47—Sample of KH158 (Towel Gourd)

FIG. 68.48—Sample of KH158 (Towel Gourd)

FIG. 69—Sample 1 KH201 Containing 18.8 g of paste of Green Mussel with 380 mL of WFI. Original plate containing cell without cell count.

FIG. 70—Sample 1 KH201 Containing 18.8 g of paste of Green Mussel with 380 mL of WFI. Cell count of 5.23 million cells.

FIG. 71—KH201 Containing 18.8 g of paste of Green Mussel with 380 mL of WFI. Cell count of 5.23 million cells.

FIG. 72—Sample number 2 KH201 with no cell count.

FIG. 73—Sample number 2 KH201 with cell count.

FIG. 74—Sample number 2 KH201 with cell count.

FIG. 75—Sample number 3 KH201 with no cell count.

FIG. 76—Sample number 3 KH201 with cell count 4.65 million.

FIG. 77—Sample number 3 KH201 with cell count 4.65 million.

FIG. 78—Sample number 4 without Tryptophan added to the medium and no cell count.

FIG. 79—Sample number 4 without Tryptophan added to the medium and with cell count of 5.53 million.

FIG. 80—Sample number 4 without Tryptophan added to the medium and with cell count of 5.53 million.

FIG. 81—Sample number 5 KH201 without Tryptophan.

FIG. 82—Sample number 5 KH201 without Tryptophan with cell count.

FIG. 83—Sample number 5 KH201 without Tryptophan with cell count.

FIG. 84—Sample number 1 KH202 (Duck) with no cell count.

FIG. 85—Sample number 1 KH202 with cell count.

FIG. 86—Sample number 1 KH202 with cell count.

FIG. 87—Sample number 2 KH202 With no cell count.

FIG. 88—Sample number 2 KH202 with cell count.

FIG. 89—Sample number 2 KH202 with cell count.

FIG. 90—Sample number 3 KH202 without cell count.

FIG. 91—Sample number 3 KH202 with cell count.

FIG. 92—Sample number 3 KH202 with cell count.

FIG. 93—Sample number 4 KH202 with no tryptophan without cell count.

FIG. 94—Sample number 4 KH202 without tryptophan with cell count.

FIG. 95—Sample number 4 KH202 without tryptophan with cell count.

FIG. 96—Sample number 5 KH202 without tryptophan with no cell count.

FIG. 97—Sample number 5 KH202 without tryptophan with cell count.

FIG. 98—Sample number 5 KH202 without tryptophan with cell count.

FIG. 99—Sample number 1 KH203 (Giant Clam) no cell count.

FIG. 100—Sample number 1 KH203 with cell count.

FIG. 101—Sample number 1 KH203 with cell count.

FIG. 102—Sample number 2 KH203 without cell count.

FIG. 103—Sample number 2 KH203 with cell count.

FIG. 104—Sample number 2 KH203 with cell count.

FIG. 105—Sample number 3 KH203 without cell count (clear solution added in the lower chamber).

FIG. 106—Sample number 3 KH203 with cell count (clear solution added in the lower chamber).

FIG. 107—Sample number 3 KH203 with cell count (clear solution added in the lower chamber).

FIG. 108—Sample 4 KH203 without tryptophan with no cell count.

FIG. 109—Sample 4 KH203 without tryptophan with cell count.

FIG. 110—Sample 4 KH203 without tryptophan with cell count.

FIG. 111—Sample 5 KH203 without tryptophan with no cell count.

FIG. 112—Sample 5 KH203 without tryptophan with cell count.

FIG. 113—Sample 5 KH203 without tryptophan with cell count.

FIG. 114—Sample KH204 (Alaskan crab) Sample #1.

FIG. 115—Sample KH204 (Alaskan crab) Sample #1.

FIG. 116—Sample KH204 (Alaskan crab) Sample #1.

FIG. 117—Sample KH204 (Alaskan crab) Sample #2.

FIG. 118—Sample KH204 (Alaskan crab) Sample #2.

FIG. 119—Sample KH204 (Alaskan crab) Sample #2.

FIG. 120—Sample KH204 (Alaskan crab) Sample #3.

FIG. 121—Sample KH204 (Alaskan crab) Sample #3.

FIG. 122—Sample KH204 (Alaskan crab) Sample #3.

FIG. 123—Sample KH204 (Alaskan crab) Sample #4.

FIG. 124—Sample KH204 (Alaskan crab) Sample #4.

FIG. 125—Sample KH204 (Alaskan crab) Sample #4.

FIG. 126—Sample KH204 (Alaskan crab) Sample #5.

FIG. 127—Sample KH204 (Alaskan crab) Sample #5.

FIG. 128—Sample KH204 (Alaskan crab) Sample #5.

FIG. 129—Sample KH205 (Pork) Sample #1.

FIG. 130—Sample KH205 (Pork) Sample #1.

FIG. 131—Sample KH205 (Pork) Sample #1.

FIG. 132—Sample KH205 (Pork) Sample #2.

FIG. 133—Sample KH205 (Pork) Sample #2.

FIG. 134—Sample KH205 (Pork) Sample #2.

FIG. 135—Sample KH205 (Pork) Sample #3.

FIG. 136—Sample KH205 (Pork) Sample #3.

FIG. 137—Sample KH205 (Pork) Sample #3.

FIG. 138—Sample KH205 (Pork) Sample #4.

FIG. 139—Sample KH205 (Pork) Sample #4.

FIG. 140—Sample KH205 (Pork) Sample #4.

FIG. 141—Sample KH205 (Pork) Sample #5.

FIG. 142—Sample KH205 (Pork) Sample #5.

FIG. 143—Sample KH205 (Pork) Sample #5.

FIG. 144—Sample KH206 (Beef) Sample #1.

FIG. 145—Sample KH206 (Beef) Sample #1.

FIG. 146—Sample KH206 (Beef) Sample #1.

FIG. 147—Sample KH206 (Beef) Sample #2.

FIG. 148—Sample KH206 (Beef) Sample #2.

FIG. 149—Sample KH206 (Beef) Sample #2.

FIG. 150—Sample KH206 (Beef) Sample #3.

FIG. 151—Sample KH206 (Beef) Sample #3.

FIG. 152—Sample KH206 (Beef) Sample #3.

FIG. 153—Sample KH206 (Beef) Sample #4.

FIG. 154—Sample KH206 (Beef) Sample #4.

FIG. 155—Sample KH206 (Beef) Sample #4.

FIG. 156—Sample KH206 (Beef) Sample #5.

FIG. 157—Sample KH206 (Beef) Sample #5.

FIG. 158—Sample KH206 (Beef) Sample #5.

FIG. 159—Sample KH207 (Mackerel Fish) Sample #1.

FIG. 160—Sample KH207 (Mackerel Fish) Sample #1.

FIG. 161—Sample KH207 (Mackerel Fish) Sample #1.

FIG. 162—Sample KH207 (Mackerel Fish) Sample #2.

FIG. 163—Sample KH207 (Mackerel Fish) Sample #2.

FIG. 164—Sample KH207 (Mackerel Fish) Sample #2.

FIG. 165—Sample KH207 (Mackerel Fish) Sample #3.

FIG. 166—Sample KH207 (Mackerel Fish) Sample #3.

FIG. 167—Sample KH207 (Mackerel Fish) Sample #3.

FIG. 168—Sample KH207 (Mackerel Fish) Sample #4.

FIG. 169—Sample KH207 (Mackerel Fish) Sample #4.

FIG. 170—Sample KH207 (Mackerel Fish) Sample #4.

FIG. 171—Sample KH207 (Mackerel Fish) Sample #5.

FIG. 172—Sample KH207 (Mackerel Fish) Sample #5.

FIG. 173—Sample KH207 (Mackerel Fish) Sample #5.

FIG. 174—Sample KH208 (Chicken) Sample #1.

FIG. 175—Sample KH208 (Chicken) Sample #1.

FIG. 176—Sample KH209 (Shrimp) Sample #1.

FIG. 177—Sample KH209 (Shrimp) Sample #1.

FIG. 178—Sample KH210 (Egg yoke) Sample #1.

FIG. 179—Sample KH210 (Egg yoke) Sample #1.

FIG. 180—Sample KH210 (Egg yoke) Sample #1.

FIG. 181—Sample KH210 (Egg yoke) Sample #2.

FIG. 182—Sample KH210 (Egg yoke) Sample #2.

FIG. 183—Sample KH210 (Egg yoke) Sample #2.

FIG. 184—Sample KH210 (Egg yoke) Sample #3.

FIG. 185—Sample KH210 (Egg yoke) Sample #3.

FIG. 186—Sample KH210 (Egg yoke) Sample #3.

FIG. 187—Sample KH210 (Egg yoke) Sample #4.

FIG. 188—Sample KH210 (Egg yoke) Sample #4.

FIG. 189—Sample KH210 (Egg yoke) Sample #4.

FIG. 190—Sample KH210 (Egg yoke) Sample #5.

FIG. 191—Sample KH210 (Egg yoke) Sample #5.

FIG. 192—Sample KH210 (Egg yoke) Sample #5.

FIG. 193—Sample KH211 (Egg white) Sample #1.

FIG. 194—Sample KH211 (Egg white) Sample #1.

FIG. 195—Sample KH211 (Egg white) Sample #1.

FIG. 196—Sample KH211 (Egg white) Sample #2.

FIG. 197—Sample KH211 (Egg white) Sample #2.

FIG. 198—Sample KH211 (Egg white) Sample #2.

FIG. 199—Sample KH211 (Egg white) Sample #3.

FIG. 200—Sample KH211 (Egg white) Sample #3.

FIG. 201—Sample KH211 (Egg white) Sample #3.

FIG. 202—Sample KH211 (Egg white) Sample #4.

FIG. 203—Sample KH211 (Egg white) Sample #4.

FIG. 204—Sample KH211 (Egg white) Sample #4.

FIG. 205—Sample KH211 (Egg white) Sample #5.

FIG. 206—Sample KH211 (Egg white) Sample #5.

FIG. 207—Sample KH211 (Egg white) Sample #5.

FIG. 208—Sample KH212 (Shanghai Crab) Sample #1.

FIG. 209—Sample KH212 (Shanghai Crab) Sample #1.

FIG. 210—Sample KH213 (Crawfish) Sample #1.

FIG. 211—Sample KH213 (Crawfish) Sample #1.

FIG. 212—Sample KH213 (Crawfish) Sample #1.

FIG. 213—Sample KH213 (Crawfish) Sample #2.

FIG. 214—Sample KH213 (Crawfish) Sample #2.

FIG. 215—Sample KH213 (Crawfish) Sample #2.

FIG. 216—Sample KH213 (Crawfish) Sample #3.

FIG. 217—Sample KH213 (Crawfish) Sample #3.

FIG. 218—Sample KH213 (Crawfish) Sample #3.

FIG. 219—Sample KH213 (Crawfish) Sample #4.

FIG. 220—Sample KH213 (Crawfish) Sample #4.

FIG. 221—Sample KH213 (Crawfish) Sample #4.

FIG. 222—Sample KH213 (Crawfish) Sample #5.

FIG. 223—Sample KH213 (Crawfish) Sample #5.

FIG. 224—Sample KH213 (Crawfish) Sample #5.

FIG. 225—Sample KH214 (Salmon Fish) Sample #1.

FIG. 226—Sample KH214 (Salmon Fish) Sample #1.

FIG. 227—Sample KH214 (Salmon Fish) Sample #1.

FIG. 228—Sample KH214 (Salmon Fish) Sample #2.

FIG. 229—Sample KH214 (Salmon Fish) Sample #2.

FIG. 230—Sample KH214 (Salmon Fish) Sample #2.

FIG. 231—Sample KH214 (Salmon Fish) Sample #3.

FIG. 232—Sample KH214 (Salmon Fish) Sample #3.

FIG. 233—Sample KH214 (Salmon Fish) Sample #3.

FIG. 234—Sample KH214 (Salmon Fish) Sample #4.

FIG. 235—Sample KH214 (Salmon Fish) Sample #4.

FIG. 236—Sample KH214 (Salmon Fish) Sample #4.

FIG. 237—Sample KH214 (Salmon Fish) Sample #5.

FIG. 238—Sample KH214 (Salmon Fish) Sample #5.

FIG. 239—Sample KH214 (Salmon Fish) Sample #5.

FIG. 240—Sample KH301 (Yonggang) Sample #1.

FIG. 241—Sample KH301 (Yonggang) Sample #1.

FIG. 242—Sample KH302 (Chinese worm medicine (Dong Chong Xia Cao)) Sample #1.

FIG. 243—Sample KH302 (Chinese worm medicine (Dong Chong Xia Cao)) Sample #1.

FIG. 244—Sample KH303 (Tibet Leave) Sample #1.

FIG. 245—Sample KH303 (Tibet Leave) Sample #1.

FIG. 246—Sample KH304 (Milk for Baby born) Sample #1.

FIG. 247—Sample KH304 (Milk for Baby born) Sample #1.

FIG. 248—Sample KH305 (Milk for three month baby) Sample #1.

FIG. 249—Sample KH305 (Milk for three month baby) Sample #1.

FIG. 250—Sample KH306 (Milk for six month baby) Sample #1.

FIG. 251—Sample KH306 (Milk for six month baby) Sample #1.

FIG. 252—Sample KH307 (Milk for 1 year old baby) Sample #1.

FIG. 253—Sample KH307 (Milk for 1 year old baby) Sample #1.

FIG. 254—Sample KH308 (Cow Milk) Sample #1.

FIG. 255—Sample KH308 (Cow Milk) Sample #1.

FIG. 256—Sample KH309 (Human Placenta) Sample #1.

FIG. 257—Sample KH309 (Human Placenta) Sample #1.

FIG. 258—Arthrosclerosis and inflammation, MMP-2 control group vs. experimental group.

FIG. 259—Arthrosclerosis and inflammation, control group vs. experimental group.

FIG. 260—Arthrosclerosis and inflammation, APOA-1 concentration vs. MMP-2 and GAPDH.

FIG. 261—Arthrosclerosis and inflammation, APOA-1 concentration vs. different receptors.

FIG. 262—Arthrosclerosis and inflammation, APOA-1 concentration vs. different receptors.

FIG. 263—KH101 through KH109 mediums vs. lung cancer cells.

FIG. 264—KH110 through KH118 mediums vs. lung cancer cells.

FIG. 265—KH119 through KH127 mediums vs. lung cancer cells.

FIG. 266—KH128 through KH206 mediums vs. lung cancer cells.

FIG. 267—KH207 through KH214 mediums vs. lung cancer cells.

FIG. 268—KH301 through KH309 mediums vs. lung cancer cells.

FIG. 268.1—KH medium with breast cancer cell.

FIG. 268.2—KH medium with high TC breast cancer cell.

FIG. 268.3—KH medium with high TC breast cancer cell.

FIG. 268.4—KH medium with Leukemia cell.

FIG. 268.5—KH medium with high TC with Leukemia cell.

FIG. 268.6—KH medium with high TC Leukemia cell.

FIG. 268.7—KH medium with lung cancer cell.

FIG. 268.8—KH medium with high TC lung cancer cell.

FIG. 268.9—KH medium with high TC lung cancer cell.

FIG. 268.10—KH135-KH149 with lung cancer cell.

FIG. 268.11—KH135-KH148 with lung cancer cell.

FIG. 268.12—KH135-KH149 with breast cancer cell.

FIG. 268.13—KH135-KH148 with breast cancer cell.

FIG. 268.14—KH135-KH149 with Leukemia cell.

FIG. 268.15—KH135-KH148 with Leukemia cell.

FIG. 268.16—KH101-KH134 medium with lung cancer cell.

FIG. 268.17—KH101-KH115 medium with lung cancer cell.

FIG. 268.18—KH116-KH131 medium with lung cancer cell.

FIG. 268.19—KH132-KH134 medium with lung cancer cell.

FIG. 268.20—KH201-KH214 medium with lung cancer cell.

FIG. 268.21—KH201-KH215 medium with lung cancer cell.

FIG. 268.22—KH216 and KH217 medium with lung cancer cell.

FIG. 268.23—KH301-KH309 medium with lung cancer cell.

FIG. 268.24—KH301-KH309 medium with lung cancer cell.

FIG. 269—FSC/SSC on FACS.

FIG. 270—FSC/SSC on FACS.

FIG. 271—FSC/SSC on FACS.

FIG. 272—FSC/SSC on FACS.

FIG. 273—FSC/SSC on FACS.

FIG. 274—FSC/SSC on FACS.

FIG. 275—FSC/SSC on FACS.

FIG. 276—FSC/SSC on FACS.

FIG. 277—FSC/SSC on FACS.

FIG. 278—Comparison with human T/B cells on FACS.

FIG. 279—Comparison with human T/B cells on FACS.

FIG. 280—Comparison with human T/B cells on FACS.

FIG. 281—Comparison with human T/B cells on FACS.

FIG. 282—Comparison with human T/B cells on FACS.

FIG. 283—Comparison with human T/B cells on FACS.

FIG. 284—Comparison with human T/B cells on FACS.

FIG. 285—Comparison with human granulocytes on FACS.

FIG. 286—Comparison with human granulocytes on FACS.

FIG. 287—Comparison with human granulocytes on FACS.

FIG. 288—Comparison with human granulocytes on FACS.

FIG. 289—Comparison with human granulocytes on FACS.

FIG. 290—Comparison with human granulocytes on FACS.

FIG. 291—Comparison with human granulocytes on FACS.

FIG. 292—Comparison with human granulocytes on FACS.

FIG. 293—Comparison with human NK cells on FACS.

FIG. 294—Total Cholesterol/cholesterol Ester quantification (TC).

FIG. 295—HDL cholesterol quantification (HDLC).

FIG. 296—LDL/VLDL cholesterol quantification (LDLC/VLDLC).

FIG. 297—Triglyceride quantification (TG).

FIG. 298—TC, HDLC and LDLC/VLDLC quantification of sample #1.

FIG. 299—TG quantification of sample#1. AFOD.

FIG. 300—TC, HDLC and LDLC/VLDLC quantification of sample #2. AFOD RAAS1.

FIG. 301—TG quantification of sample #2. AFOD RAAS1.

FIG. 302—TC, HDLC and LDLC/VLDLC quantification of sample #3. AFOD RAAS2.

FIG. 303—TG quantification of sample #3. AFOD RAAS2.

FIG. 304—TC, HDLC and LDLC/VLDLC quantification of sample #4. AFCC RAAS1.

FIG. 305—TG quantification of sample #4. AFCC RAAS1.

FIG. 306—TC, HDLC and LDLC/VLDLC quantification of sample #5. AFCC RAAS2.

FIG. 307—TG quantification of sample #5. AFCC RAAS2.

FIG. 308—TC, HDLC and LDLC/VLDLC quantification of sample #6. AFCC RAAS3.

FIG. 309—TG Quantification of sample #6. AFCC RAAS3.

FIG. 310—TC, HDLC and LDLC/VLDLC quantification of sample #7. AFCC RAAS4.

FIG. 311—TG quantification of sample #7. AFCC RAAS4.

FIG. 312—TC, HDLC and LDLC/VLDLC quantification of sample #8. AFCC RAAS5.

FIG. 313-TG quantification of sample #8. AFCC RAAS5.

FIG. 314—TC, HDLC and LDLC/VLDLC quantification of sample #9. AFOD RAAS3.

FIG. 315—TG quantification of sample #9. AFOD RAAS3.

FIG. 316—TC, HDLC and LDLC/VLDLC quantification of sample #12. RE-VIII RAAS

FIG. 317—TG quantification of sample #12. RE-VIII RAAS.

FIG. 318—Standard curve of Total Cholesterol/Cholesterol Ester Quantification (TC) FIG. 319—Standard curve of HDL Cholesterol Quantification (HDLC).

FIG. 320—Standard curve of LDL/VLDL Cholesterol Quantification (LDLC/VLDLC) FIG. 321—Standard curve of Triglyceride Quantification (TG).

FIG. 322—Quantification of TC, HDL, LDL/VLDL and TG of sample KH 101.

FIG. 323—Quantification of TC, HDL, LDL/VLDL and TG of sample KH 102.

FIG. 324—Quantification of TC, HDL, LDL/VLDL and TG of sample KH 103.

FIG. 325—Quantification of TC, HDL, LDL/VLDL and TG of sample KH 104.

FIG. 326—Quantification of TC, HDL, LDL/VLDL and TG of sample KH 105.

FIG. 327—Quantification of TC, HDL, LDL/VLDL and TG of sample KH106.

FIG. 328—Quantification of TC, HDL, LDL/VLDL and TG of sample KH 107.

FIG. 329—Quantification of TC, HDL, LDL/VLDL and TG of sample KH 108.

FIG. 330—Quantification of TC, HDL, LDL/VLDL and TG of sample KH 109.

FIG. 331—Quantification of TC, HDL, LDL/VLDL and TG of sample KH 110.

FIG. 332—Quantification of TC, HDL, LDL/VLDL and TG of sample KH 111.

FIG. 333—Quantification of TC, HDL, LDL/VLDL and TG of sample KH 112.

FIG. 334—Quantification of TC, HDL, LDL/VLDL and TG of sample KH 113.

FIG. 335—Quantification of TC, HDL, LDL/VLDL and TG of sample KH 114.

FIG. 336—Quantification of TC, HDL, LDL/VLDL and TG of sample KH 115.

FIG. 337—Quantification of TC, HDL, LDL/VLDL and TG of sample KH 116.

FIG. 338—Quantification of TC, HDL, LDL/VLDL and TG of sample KH 117.

FIG. 339—Quantification of TC, HDL, LDL/VLDL and TG of sample KH 118.

FIG. 340—Quantification of TC, HDL, LDL/VLDL and TG of sample KH 119.

FIG. 341—Quantification of TC, HDL, LDL/VLDL and TG of sample KH 120.

FIG. 342—Quantification of TC, HDL, LDL/VLDL and TG of sample KH 121.

FIG. 343—Quantification of TC, HDL, LDL/VLDL and TG of sample KH 122.

FIG. 344—Quantification of TC, HDL, LDL/VLDL and TG of sample KH 123.

FIG. 345—Quantification of TC, HDL, LDL/VLDL and TG of sample KH 124.

FIG. 346—Quantification of TC, HDL, LDL/VLDL and TG of sample KH 125.

FIG. 347—Quantification of TC, HDL, LDL/VLDL and TG of sample KH 126.

FIG. 348—Quantification of TC, HDL, LDL/VLDL and TG of sample KH 127.

FIG. 349—Quantification of TC, HDL, LDL/VLDL and TG of sample KH 128.

FIG. 350—Quantification of TC, HDL, LDL/VLDL and TG of sample KH 129.

FIG. 351—Quantification of TC, HDL, LDL/VLDL and TG of sample KH 130.

FIG. 352—Quantification of TC, HDL, LDL/VLDL and TG of sample KH 131.

FIG. 353—Quantification of TC, HDL, LDL/VLDL and TG of sample KH 132.

FIG. 354—Quantification of TC, HDL, LDL/VLDL and TG of sample KH 133.

FIG. 355—Quantification of TC, HDL, LDL/VLDL and TG of sample KH 134.

FIG. 356—Quantification of TC, HDL, LDL/VLDL and TG of sample KH 201.

FIG. 357—Quantification of TC, HDL, LDL/VLDL and TG of sample KH 202.

FIG. 358—Quantification of TC, HDL, LDL/VLDL and TG of sample KH 203.

FIG. 359—Quantification of TC, HDL, LDL/VLDL and TG of sample KH 204.

FIG. 360—Quantification of TC, HDL, LDL/VLDL and TG of sample KH 205.

FIG. 361—Quantification of TC, HDL, LDL/VLDL and TG of sample KH 206.

FIG. 362—Quantification of TC, HDL, LDL/VLDL and TG of sample KH 207.

FIG. 363—Quantification of TC, HDL, LDL/VLDL and TG of sample KH 208.

FIG. 364—Quantification of TC, HDL, LDL/VLDL and TG of sample KH 209.

FIG. 365—Quantification of TC, HDL, LDL/VLDL and TG of sample KH 210.

FIG. 366—Quantification of TC, HDL, LDL/VLDL and TG of sample KH 211.

FIG. 367—Quantification of TC, HDL, LDL/VLDL and TG of sample KH 212.

FIG. 368—Quantification of TC, HDL, LDL/VLDL and TG of sample KH 213.

FIG. 369—Quantification of TC, HDL, LDL/VLDL and TG of sample KH 214.

FIG. 370—Quantification of TC, HDL, LDL/VLDL and TG of sample KH 215.

FIG. 371—Quantification of TC, HDL, LDL/VLDL and TG of sample KH 216.

FIG. 372—Quantification of TC, HDL, LDL/VLDL and TG of sample KH 217.

FIG. 373—Quantification of TC, HDL, LDL/VLDL and TG of sample KH 301.

FIG. 374—Quantification of TC, HDL, LDL/VLDL and TG of sample KH 302.

FIG. 375—Quantification of TC, HDL, LDL/VLDL and TG of sample KH 303.

FIG. 376—Quantification of TC, HDL, LDL/VLDL and TG of sample KH 304.

FIG. 377—Quantification of TC, HDL, LDL/VLDL and TG of sample KH 305.

FIG. 378—Quantification of TC, HDL, LDL/VLDL and TG of sample KH 306.

FIG. 379—Quantification of TC, HDL, LDL/VLDL and TG of sample KH 307.

FIG. 380—Quantification of TC, HDL, LDL/VLDL and TG of sample KH 308.

FIG. 381—Quantification of TC, HDL, LDL/VLDL and TG of sample KH 309.

FIG. 382—Standard curve of Total Cholesterol/Cholesteryl Ester Quantification (TC) FIG. 383—Standard curve of HDL Quantification.

FIG. 384—Standard curve of HDL Quantification.

FIG. 385—Standard curve of LDL/VLDL Quantification.

FIG. 386—Standard curve of LDL/VLDL Quantification.

FIG. 387—Standard curve of Triglyceride Quantification (TG).

FIG. 388—Standard curve of Triglyceride Quantification (TG).

FIG. 389—Shanghai Daily report from Sep. 20, 2012 on genetic modified corn. FIG. 390—Different cancer cells cultured with HEK 293 cell CCK8 result.

FIG. 391—Different cancer cells culture.

FIG. 392—Different cancer cells cultured with HEK293 cell.

FIG. 393—Effects of AFOD KH, AFOD 103, AFOD 107, AFOD 108 and AFOD 1 on body weight (A) and body weight change (B) in AIA model till Day 35 (*p<0.05, **p<0.01, ***p<0.001, treatment groups v.s. saline group, two-way repeated or one-way ANOVA).

FIG. 394—Effects of AFCC KH, AFOD 101 and AFOD 102 on body weight (A) and body weight change (B) in AIA model till Day 45 (**p<0.01, ***p<0.001, treatment groups v.s. saline group, two-way repeated or one-way ANOVA).

FIG. 395—Effects of AFOD KH, AFOD 103, AFOD 107, AFOD 108 and AFOD 1 on delta paw (right hind paw) volume (A) in AIA model till Day 35. AUC of delta paw volume curves were also presented (B). The delta paw volume of Dex group was significantly lower than saline group, from day 14 (***p<0.001, v.s. saline group, two-way repeated or one-way ANOVA).

FIG. 396—Effects of AFCC KH, AFOD 101 and AFOD 102 on delta paw (right hind paw) volume (A) in AIA model till Day 45. AUC of delta paw volume curves were also presented (B). The delta paw volume of Dex group was significantly lower than saline group, from day 14 (***p<0.001, v.s. saline group, two-way repeated or one-way ANOVA).

FIG. 397—Effects of AFOD KH, AFOD 103, AFOD 107, AFOD 108 and AFOD 1 on arthritic score in AIA model till day 35. The arthritic score of Dex group was significantly lower than saline group, from day 14 (p<0.01 for day 14, p<0.001 for day 16 to 35, Kruskal-Wallis test).

FIG. 398—Effects of AFCC KH, AFOD 101 and AFOD 102 on arthritic score in AIA model till Day 45. The arthritic score of Dex group was significantly lower than saline group, from day 14 (p<0.01 for day 14, p<0.001 for day 16 to 45, Kruskal-Wallis test).

FIG. 399—Effects of AFOD KH, AFOD 103, AFOD 107, AFOD 108 and AFOD 1 on incidence rate in AIA model till day 35. The incidence rate reached 100%, 11 days after immunization. There was no change of incidence rate afterward, for all the treatment groups.

FIG. 400—Effects of AFCC KH, AFOD 101 and AFOD 102 on incidence rate in AIA model till day 45. The incidence rate reached 100%, 11 days after immunization. There was no change of incidence rate afterward, for all the treatment groups.

FIG. 401—Efficacy of therapeutic treatment or prophylactic treatment of RAAS 8 or ETV on in vivo HBV replication in HBV mouse HDI model

FIG. 402—Effect of prophylactic treatment or therapeutic treatment of RAAS 8 or ETV on the HBsAg in mouse blood.

FIG. 403—. Effect of prophylactic treatment or therapeutic treatment of RAAS 8 or ETV on the intermediate HBV replication in the mouse livers by qPCR.

FIG. 404—Southern blot determination of intermediate HBV DNA in mouse livers.

FIG. 405—The body weights of mice treated with vehicle or indicated compounds during the course of experiment.

FIG. 406—CD3+ T lymphocytes in lymph node.

FIG. 407—T lymphocytes subsets in lymph node.

FIG. 408—Dendritic cell in lymph node.

FIG. 409—CD4+ T lymphocytes subsets in lymph node.

FIG. 410—CD8 T lymphocytes subsets in lymph node.

FIG. 411—Macrophage/Granulocytes in lymph node.

FIG. 412—T regulate cells in lymph node.

FIG. 413—T lymphocytes/B lymphocytes in spleen.

FIG. 414—Dendritic cell subsets in spleen.

FIG. 415—CD4+ T lymphocytes subsets in spleen.

FIG. 416—CD8 T lymphocytes subsets in spleen.

FIG. 417—Macrophages subsets in spleen.

FIG. 418—Macrophages/Granulocytes in spleen.

FIG. 419—T regulate cells in spleen.

FIG. 420—T lymphocytes/B lymphocytes in peripheral blood.

FIG. 421—T lymphocytes subsets in peripheral blood.

FIG. 422—Granulocytes/Dendritic cells in peripheral blood.

FIG. 423—Monocytes in peripheral blood.

FIG. 424—CD3+ T lymphocytes in lymph node.

FIG. 425—T lymphocytes subsets in lymph node.

FIG. 426—Dendritic cell in lymph node.

FIG. 427—CD4+ T lymphocytes subsets in lymph node.

FIG. 428—CD8 T lymphocytes subsets in lymph node.

FIG. 429—Macrophages/Granulocytes in lymph node.

FIG. 430—T regulate cells in lymph node.

FIG. 431—T lymphocytes/B lymphocytes in spleen.

FIG. 432—T lymphocytes subsets in spleen.

FIG. 433—Dendritic cell subsets in spleen.

FIG. 434—CD4+ T lymphocytes subsets in spleen.

FIG. 435—CD8 T lymphocytes subsets in spleen.

FIG. 436—Macrophages subsets in spleen.

FIG. 437—Macrophages/Granulocytes in spleen.

FIG. 438—T regulate cells in spleen.

FIG. 439—T lymphocytes/B lymphocytes in peripheral blood.

FIG. 440—T lymphocytes subsets in peripheral blood.

FIG. 441—Granulocytes/Dendritic cells in peripheral blood F.

FIG. 442—Monocytes in peripheral blood.

FIG. 443—Effect of APOA1 on body weight.

FIG. 444—Plasma lipid profile of ApoE mice fed with a normal diet and high fat diet.

FIG. 445—Effect of RAAS antibody on plasma total cholesterol.

FIG. 446—Net change of RAAS antibody on plasma total cholesterol.

FIG. 447—The effect of RAAS antibody on total plasma Triglyceride.

FIG. 448—The effect of RAAS antibody on High Density Lipoprotein.

FIG. 449—Net change of RAAS antibody on High Density Lipoprotein.

FIG. 450—The effect of RAAS antibody on Low Density Lipoprotein.

FIG. 451—Net change of RAAS antibody on Low Density Lipoprotein.

FIG. 452—Effect of RAAS antibody on negative control group on Atherosclerosis plaque lesion.

FIG. 453—Percent of plaque area in total inner vascular area.

FIG. 454—Illustrated analysis of arterial arch area.

FIG. 455—Percent of plaque area in the arterial arch area.

FIG. 456—Illustrated analysis from root to right renal artery.

FIG. 457—Percent of plaque area from root to right renal artery.

FIG. 458—Diagram of liver weight.

FIG. 459—Diagram of liver index.

FIG. 460—Comparison of percentage of plaque area in study 1, 2, 3.

FIG. 461—Comparison of Total Cholesterol level in study 1, 2, 3.

FIG. 462—Comparison of percentage of plaque area in study 1, 2, 3.

FIG. 463—Images of aorta plaque lesions after 16 weeks treatment.

### INVENTION: GOOD HEALTHY CELLS

### Dragon Cell:

On Aug. 23, 2011, we began to culture the first plate with the cryoprecipitate poor plasma Fractionation Lot: 20110810-4B consisting of the following three plasma stations, collection date and weight of the plasma.

| Plasma station | Collected date | Weighted |
|---|---|---|
| Dahua, Guangxi | Aug. 11, 2010~Apr. 7, 2011 | 1.77 tons |
| Shimen, Hunan | Aug. 27, 2010~Dec. 17, 2010 | 1.40 tons |
| Wumin, Guangxi | Dec. 23, 2010~Apr. 11, 2011 | 1.63 tons |

With the total of 4,800 liters of plasma from 12,800 donations containing WHITE

BLOOD CELLs as Red Blood cells were returned to Donor through Plasmapheresis, from the healthy Chinese donors who have been tested negative for HBV, HCV and HIV and the other required test for plasma donation. The donors are mainly repeat donors, mostly farmers who have a very active and stress free lifestyle and an ideal diet, consisting of more vegetables from Guangxi province and Hunan province.

According to the Gerontological Society of China we have found that the oldest person in China is in Guangxi province at the age of 129 years old.

After centrifugation the paste and supernatant were used to culture on Aug. 20, 2011. We used a 24-well plate. These kind of plates are made of polystyrene. These that are designed for adherent cells have been treated chemically to promote cell adhesion and are called tissue culture dishes.

| | Diameter | Growth area |
|---|---|---|
| | of each well | of each well |
| 6-well plate | 34.8 mm | 9.5 cm2 |
| 12-well plate | 22.1 mm | 3.8 cm2 |
| 24-well plate | 15.6 mm | 1.9 cm2 |
| 48-well plate | 11.0 mm | 0.95 cm2 |
| 96-well plate | 6.4 mm | 0.32 cm2 |

Each well can contain a maximum 2,000 micro liters of the medium. This plate contains the cells that live and grow until Jan. 25, 2012 when we wrote this invention for patent. 5 months and 5 days when most scientists conclude that the cell will live only for 7 days in a culture medium.

From day 1 to day 21 just a few pictures have been taken from microscope and on the 21^{st} day when being asked by the inventor the progress of the cell culture by the inventor the scientist report that they are not cells, only the fragment of dead cells. And she has used the trypan blue dye to see if the cells are alive or dead. She concluded that they were all dead fragments of cells, at this time from day 21 the inventor himself got heavily involved through the microscope the growth of the cell. The cell then begin to grow with the different shape just like described in the tittle of this patent. The inventor believes that these are living cells.

The scientist conducting the experiment thinks the findings were fibers or miscellaneous fragments stuck at the bottom of the well, but not living cells. The inventor ordered the scientist to use the pipette to stir violently the bottom of the plate, to destroy everything in that well, then to transfer half of the medium into two more plates (Plate 2 and Plate 3). The new found Dragon cell in plate #2, well #5 out of 5 culture plates on day 31. In order to prove that this is a living cell the inventor has been monitoring the Dragon well very closely on a daily basis and found different movement patterns from the Dragon cell. During a 12 minute video the inventor has observed the Dragon cell move up and down, appear and disappear. We have not yet observed the Dragon cell in our protein products. However we have observed the same other cells as the ones in the Dragon well #5. The physical description of the Vietnamese Dragon fit with the description of the Dragon cell that we discovered. The Vietnamese Dragon does not have a beard and no horns. Its tongue is thin and narrow and long, it has big eyes and his jaw opens wide so his teeth show. It's nose is in perfect shape, unlike the Chinese Dragon. The Vietnamese Dragon holds a jade in his mouth, while the Japanese, Korean and Chinese Dragons hold the same jade in the leg. (According to VIEN DONG DAILY NEWS 2012, the Year of Dragon addition)

In the transferred medium from the original plate well #5 we discovered the snake cell, also the medium from this well transferred into well #5 of the second plate is where the Dragon appeared.

### Snake Cell:

In the western culture in history the theory one say that the Dragon always with the snake. That is true in the theory in the east as well. French language in the beginning 13^{th} centuries (much later than China and Vietnam) called Dragon as DRAGE from Latin language: Draconem and it also has the meaning. A BIG SNAKE. Egyptian language called DRAKON, which means SNAKE or a GIANT WATER SNAKE. English language: DRAGON came from DRA'KO'N of Greece which also means a very long Water Snake.

We have not observed the Snake in our products, however we have observed the snake in the nude mice with Breast cancer that have been treated with our products, AFCC and AFOD. The DNA is obvious seen inside the body of the Snake cell. It has been observed that the Snake cell just like the characteristics of the other cells has changed shapes and sizes. The description of each cell was obtained by observing from thousands of hours of video and still pictures. The observation still continues to obtain the behavior of this cell and to find how long they can live in a cultured medium.

### Double Ring Cell:

This type of cell is the most active we have observed in our products. The cell consists of two rings, smaller ring in the inside and a larger one on the outside. The size of the double ring cell varies keeping the same structure. We documented this type of cell moving at different speeds at different times sending a beaming signal from the outer ring. Sometimes they move alone and at other times they move in groups in different directions with in the well. The description of each cell was obtained by observing from thousands of hours of video and still pictures. The observation still continues to obtain the behavior of this cell and to find how long they can live in a cultured medium.

### Lighting Cell:

This type of cell has been observed moving much like a thunderstorm. Spreading lighting very quickly. The shape resembles a cluster of cells changing shape as it moves. The description of each cell was obtained by observing from thousands of hours of video and still pictures. The observation still continues to obtain the behavior of this cell and to find how long they can live in a cultured medium.

### Square Pixel Cell:

This type of cell is much smaller than the others, the shape resembles that of a square block and it moves in a cluster signaling from on to the others changing the background of the cell at the bottom of the plate. The description of each cell was obtained by observing from thousands of hours of video and still pictures. The observation still continues to obtain the behavior of this cell and to find how long they can live in a cultured medium.

### Beaming Rays Cell:

This type of cell was observed displaying different brightness as it moved very slowly. The shape changed from a round structure to an oval shaped structure. The lighting of the cell replicated that of continuous beaming yellow light. The description of each cell was obtained by observing from thousands of hours of video and still pictures. The observation still continues to obtain the behavior of this cell and to find how long they can live in a cultured medium.

### Reconstruction Background Cell:

This type of cell was observed changing the background cells by changing layer after layer of the cluster of cells when we observed the Dragon cell move. The description of each cell was obtained by observing from thousands of hours of video and still pictures. The observation still continues to obtain the behavior of this cell and to find how long they can live in a cultured medium.

### Crater Cell

This type of cell was observed in the culture medium at the bottom of the well. We did not observe any movement. The structure resembles the shape of a volcano crater.

### Yellow Cell

This type of cell was observed in the culture medium at the bottom of the well. We did not observe any movement. This yellow cell in CHO cell made movement.

### Facet Cell

This type of cell was observed moving in the culture plate. The structure resembles that of a human being face, having two eyes, nose and a mouth.

### Leer Cell

This type of cell was observed in 10 year old Human Albumin. The cell was observed moving slowly and it resembled the shape of a leer.

### Good Healthy Cells Size:

Usually the size of cells which have been discovered have a smaller size of the four micrometer. Based on the filter that we used to filter the Cryoprecipitate poor pool of plasma the size is 0.22 micrometers and for the protein product we go through the 0.22—micrometer then onto 20—nanometer virus removal the cell also can pass through with the protein. So all size of the cell discovered are much smaller than 20-nanometer. Usually people including the health authorities thought that the cell cannot go through the small size of filter such as 20-nanometer and the cell membrane have been stripped off leaving only protein going through the filter, therefore they thought that only protein was present in the product but not the cell. The inventor discovered that this types of cells can go through the 20 nanometer and can survive during the process of manufacturing from 6,000 rpm centrifugation, up to 40% of alcohol addition into the plasma together with virus inactivation just like solvent detergent technology, pasteurization, double pasteurization, heating up to 100° C., 20 nanometer filtration and other additional steps of filtering during the ultra filtration with different sizes of filters. All these cells can LIVE in lyophilized form or in liquid form and can live up to ten years back from 2012 in AlbuRAAS® (HumanAlbumin) and GammaRAAS® (Intravenous Immune Globulin)

### The Cells Must be Good and Healthy Containing the Good Proteins Inside, do not Die and Survive and are Present in the Products

In order to prove the existence of cells in the product, we have cultured the product and we immediately found the presence of the living cells. These GOOD HEALTHY cells can live outside of the body in the plasma, fraction paste and products for a long time.

### Mechanism of Good Healthy Cells:

Being GOOD HEALTHY CELLS, The cells must have A NORMAL GENE (DNA), which can transcribe into the RNA. This RNA is then subject to post-transcriptional modification and control, resulting in a mature mRNA that then is transported out of the nucleus and into the cytoplasm, where it undergoes translation into a protein. This protein from the good healthy cell can help transform the bad cell into the good healthy cell to fight the diseases, cancers, bacteria, viruses, neurological diseases, provide coagulation factors (to the point that Hemophiliac patients can produce coagulant factors for themselves), to regulate and restore the metabolism for the pancreas to produce the insulin for diabetics, send the recognition signal to people suffering from Alzheimer, Parkinson disease and Autism.

### Efficacy of the Good Healthy Cells:

A combination of 26 proteins in the AFCC (Under a separated patent application For 16 Processes to manufacture AFCC) consisting of: —C3 Complement C3 ENO1 Isoform-ENOL Isoform-TUFM elongation factor-ASS1 Argininosuccinate-ASS1 Argininosuccinate-ANXA2 Isoform 2 of Annexin A2-Glyceraldehyde-3-phosphate dehydrogenase-Glyceraldehyde-3-phosphate dehydrogenase-Glyceraldehyde-3-phosphate dehydrogenase-ANXA2 Isoform 2 of Annexin A2 KRT 86 Keratin, type II cuticular HB6-Glyceraldehyde-3-phosphate dehydrogenase-Glyceraldehyde-3-phosphate dehydrogenase-KH 20 Protein-LDHA Isoform 1 of L-lactate dehydrogenase A chain-Fibrin beta—KH 21Protein-Growth-inhibiting protein 25-Fibrinogen gama-Chain L, Crystal structure of Human Fibrinogen-Growth-inhibiting protein 25 Chain A of IgM-Chain A Crystal structure of the Fab fragment of A Human Monoclonal Igm Cold Agglutinin-Immunoglobulin light chain-Chain C, Molecular Basis for Complement Recoginition has been used to cure nude mice with breast cancer for a period of 77 days. The tumor size of this nude mice #3-7 has gone from 0 to 5,650 down to 4,935 and at this point the tumor detached from the body and the wound is in the process of healing. On Nov. 9, 2011 the nude mice #3-7 due to bad animal care of the CRO lab, the inventor decided to sacrifice the remaining group of animals including mice #3-7 then brought this mice over to another CRO lab for further studies using the tissue surrounding the tumor wound and cut 20 mm3 fragments to implant into 10 new nude mice to see if the tumor still grow.

Some of the mice grew the tumor size up to about 400 mm3 and eventually disappeared. CRO reported that this mice was infected but did not show any sign of infection.

AFCC is also known to kill viruses like H1, N1, HBV, HCV, and HIV as well as Bacteria. Therefore it is impossible that this mice has been infected.

The recovery and the reduction of tumor size was due to GOOD HEALTHY CELLS with their proteins (including two new found ones named KH20 and KH21 under a different patent application for 28 New found proteins with their sequence) providing signal to the DNA which trigger the RNA to copy the good healthy protein. This has been proven in the culture of the tumor that popped out from its body on Oct. 19, 2011.

On Oct. 23, 2011 we used a piece of this tumor that has been detached from its body and cultured it.

Oct. 26, 2011 we obtained the picture of the Snake GOOD HEALTHY cell with its DNA similar to a lot of GOOD HEALTHY Snake cells that we have obtained from other culture plates.

On Nov. 28, 2011 we observed the appearance of another GOOD HEALTHY Double ring cell.

On Dec. 16, 2011 we observed the well again and we discovered another different shape of the GOOD HEALTHY Snake cell.

On Jan. 26, 2012 taking a picture of the same plate and we observed a different form of GOOD HEALTHY cells.

Also on this date, Jan. 26, 2012, the inventor re cultured a little piece of the same tumor from mice #3-7 and found a GOOD HEALTHY Snake cell again.

This proves that the AFCC has signaled to change the DNA of this breast cancer cell from nude mice #3-7 and transformed the RNA into GOOD HEALTHY cell, mainly the Snake cell, containing GOOD HEALTHY protein.

AFOD A combination of the 15 Proteins—(16 Processes for the manufacture of AFOD is under a separated patent application) consisting of: —CP 98 kDa protein-CP Reuloplasmin—KRT2 Keratin, type II cytoskeletal epidermal-KH 22 Protein-KH 23

Protein-KH 24 Protein-KH 25 Protein (New found proteins among 28 new discovered proteins under a separated patent application)-APOA1 Apolipoprotein A-1—APOA1

Apolipoprotein A-1—APOA1 Apolipoprotein A-1—APOA1 Apolipoprotein A-1—Human Albumin-Transferrin-Vimentin-Haptoglobin has been used in a pilot study for Nude mice N 4-6 which has been cured by AFOD within one month with a tumor size up to 2562 mm3 down to almost 0 and 4-6 mice which has Been recovered completely from Breast cancer, GREW HAIR on its HEAD after Aug. 31, 2011 This nude mice has been living well until NOV 9 when It was sacrificed and his body brought to another CRO for further study. On November 11, Fragments of 20 mm3 from its body were implanted into another 9 Nude Mice to see if the Breast cancer tumor grow, until NOW Jan. 27, 2011 There is Breast Cancer Tumor GROWTH in this Nude mice 4-6.

Tissue from this Nude mice 4-6 was used to culture and grew with GOOD HEALTHY CELL not BREAST CANCER CELL any more. ANIMAL CARE and TREATMENT after Breast tumor have been detached from their body: Our phathologist and surgeon have been involved with CRO to check their Health condition on daily basis as a patient. All Nude mice whose tumor have been detached, Their wounds were cleaned daily and antibiotics applied.

Through this initial PILOT STUDY, It is found that:

Within three weeks, for a Breast cancer Nude mice could not be CURED for A Protein or combination of Proteins like AFCC and AFOD. The limit of 2000 MM3 measurement of the tumor size will also lead to Faulty Conclusion of CRO about the efficacy of these proteins.

In this PILOT study, It is found that the shortest duration for A nude mice To recover from Breast cancer, the timing is about 1 month and the Tumor Size could reach to 2500 MM3 for the Case of Nude Mice Nr 4-6 With the case of Nude mice 3-7 Tumor size could reach 6000 to 7000 MM3 For it to be detached from its body and on the way to recover and the timing for the treatment is nearly 3 months.

### The Life of Good Healthy Cells:

These good healthy cells in culture were obtained from Aug. 11, 2010 plasma products. In at least 50 plates and these cells are still alive until today Jan. 26, 2012 when we wrote this patent.

In order to determine how long these good healthy cells can live we are now culturing lots of plasma (5, 10 years old and current) and the product (5, 10 years old and current) and Fraction IV dating back to 1994. Regarding the Dragon cell we believe this cell belong one of the donor who has this characterized gene. Attempts have been made to culture the Dragon cell but so far we have not succeeded and we only have one Dragon cell. In order to determine if we can reproduce the Dragon Cell, we are culturing the same lot of plasma to see if we can find the Dragon again. This Dragon cell may represent longevity with a very healthy life.

These GOOD HEALTHY cells can live out of the human body (plasma, fraction paste and products) in different temperature conditions from −25° C. to 100° C. and may live as long as 10 years in plasma products and 15 years in fraction IV and possibly even longer.

To prove these GOOD HEALTHY CELLS live this long in our products, On Jan. 27, 2012

We cultured 2 Lots each of:

AlbuRAAS® (Human Albumin) Lot 2002038AO manufactured in 2002 (expired in 2007), now until March 2012 it will be 10 years. Lot 200701A001 Manufactured in 2007 now 5 years and expired.

GammaRAAS® (Intravenous Immune Globulin) Lot Number 20031211 manufactured in 2003 Now 9 Years. Lot Number 200701G003 Expired Now 5 years.

The evidence of GOOD HEALTHY CELLS's presence is Clear. GOOD HEALTHY CELLS ARE LIVING and MOVING in the wells of these plate.

Conclusion: GOOD HEALTHY CELLS with GOOD PROTEINS could live beyond 10 years time and we are continuing the discovery to see how long they can live.

### In Vitro Study:

GOOD HEALTHY CELLS containing Good Proteins transformed DNA of H1, N1 Virus, Hepatitis B, and RNA of Hepatitis C, and HIV viruses. Study was performed at one of the top ten CRO.

### I. Study Objective:

### HCV Study

To analyze human plasma derived proteins for anti-HCV activity (EC₅₀) and cytotoxicity (CC₅₀) using HCV 1a, 1b and 2a replicon culture systems

### II. Study Protocols

### 1. Materials:

### 1.1 Cell Line:

Replicon cell lines 1a and 2a were established following published methods (1,2) using Huh7 by G418 selection. The replicons were assembled using synthetic gene fragments. The GT 1a line is derived from H77 and contains PVIRES-Luciferase-Ubi-Neo, and two adaptive mutations: P1496L, S2204I. The 2a line contains no adaptive mutations and encodes a Luciferase reporter. The 1b replicon plasmid is also assembled using synthetic gene fragments. The replicon genome contains PVIRES-Luciferase Ubi-Neo gene segments and harbors 1 adaptive mutation (S2204I), and the backbone is Con1.

### 1.2 Compounds:

The test articles are supplied in the form of dry powder or 10 mM solution, and Ribavirin as control, in duplicate.

### 1.3 Reagents:

TABLE 1
List of reagents

| | | Catalog |
|---|---|---|
| Reagent | Vendor | Number |
| Dimethyl sulfoxide (DMSO) | Sigma | Cat#34869 |
| DMEM | Invitrogen | Cat#11960-044 |
| Fetal Bovine Serum (FBS) | Gibco | Cat#16140 |
| Penicillin-Streptomycin | Invitrogen | Cat#15070063 |
| MEM non-essential amino acids | Invitrogen | cat#11140-050 |
| L-Glutamine | Invitrogen | Cat#25030-081 |
| Trypsin/EDTA | Invitrogen | Cat#25200-072 |
| DPBS/Modified | Hyclone | SH30028.01B |
| 96 well cell plate | Greiner | Cat#655090 |
| CellTiter fluor | Promega | Cat#G6082 |
| Bright-Glo Promega Cat#E2650 | | |

### 1.4 Instrument

Envision (Perkinelmer)
Multidrop (Thermo)
Janus (Perkinelmer)

### 2. Methods

### 2.1 Cell Addition

T150 flask containing 1a, 1b and 2a replicons cell monolayer is rinsed with 10 ml pre-warmed PBS. Add 3 ml of pre-warmed Trypsin 0.25% and incubate at 5% CO₂, 37° C. for 3 minutes. Nine milliliters of DMEM complete media are added, and the cells are blown for 30 s by pipetting. The cells are counted using hemocytometer.

1a, 1b and 2a replicons cells are resuspended in medium containing 10% FBS to reach a cell density of 64,000 cells/ml (to obtain a final cell plating density of 8000 cells/125 ul/well). Plate cells in Greiner 96 black plate using Multidrop. Incubate plate at 5% CO₂, 37° C. for 4 hours.

### 2.2 Compound Addition

RAAS provided the test articles in the form of dry powder or liquid (Table 2). Test samples were diluted in PBS as 3.5×10⁴ μg/ml stocks. Sample dilutions are made by Janus with 2-fold serial dilutions for 10 concentrations plus PBS. Ribavirin is also diluted by Janus with 2-fold for 10 concentrations. The final sample concentrations of the HCV replicon assay are described in Table 3.

TABLE 2
Sample information

| Name | Protein conc. | Formulation | Diluents |
|---|---|---|---|
| AFOD KH | 10% | Liquid | |
| AFCC KH | 3.50% | Liquid | |
| AFCC RAAS 1 | 4% | Lyophilized | AFOD KH 10 mL |
| AFCC RAAS 4 | 0.0020% | Lyophilized | AFOD KH 10 mL |
| AFCC RDNA | 0.00001% | Lyophilized | AFOD KH 10 mL |

TABLE 3
Sample or compound concentrations for EC₅₀ and CC₅₀ measurement

| Name | HCV | | | | | | | | | | |
|---|---|---|---|---|---|---|---|---|---|---|---|
| | | Concentration (μg/ml) | | | | | | | | | |
| AFOD KH | 1a/1b/2a | 400 | 200 | 100 | 50 | 25 | 12.5 | 6.3 | 3.1 | 1.6 | 0.8 |
| AFCC KH | | 400 | 200 | 100 | 50 | 25 | 12.5 | 6.3 | 3.1 | 1.6 | 0.8 |
| AFCC RAAS 1 | | 400 | 200 | 100 | 50 | 25 | 12.5 | 6.3 | 3.1 | 1.6 | 0.8 |
| AFCC RAAS 4 | | 400 | 200 | 100 | 50 | 25 | 12.5 | 6.3 | 3.1 | 1.6 | 0.8 |
| AFCC RDNA | | 400 | 200 | 100 | 50 | 25 | 12.5 | 6.3 | 3.1 | 1.6 | 0.8 |

| Concentration (μM) | | | | | | | | | |
|---|---|---|---|---|---|---|---|---|---|
| 320 | 160 | 80 | 40 | 20 | 10 | 5 | 2.5 | 1.3 | 0.6 |

2.3 Detection (after 72 Hours of Incubation)

Bright-Glo Luiferase and CellTiter-Fluor™ are prepared and stored in dark while allowing to equilibrate to room temperature. Plates are removed from incubator to allow equilibration to room temperature. Multidrop is used to add 40 ul CellTiter-Fluor™ to each well of compound-treated cells. The plates are incubated for 0.5 hour, and then read on an Envision reader for cytotoxicity calculation. The cytotoxicity is calculates using the equation below.

100 ul of Bright-Glo are added to each well, incubated for 2 minutes at room temperature, and chemi-luminescence (an indicator of HCV replication) is measured for EC₅₀ calculation.

The anti-replicon activity (% inhibition) is calculated using the equation below

Dose-response curves are plotted using Prism.

### 1 Assay Plate Map

plate 1. column column column column column column
column column column column column column

| | | |
|---|---|---|
| 1 | 2 3 4 5 6 7 8 9 10 11 12 | |
| 1000000 | 1000000 1000000 1000000 1000000 1000000 1000000 1000000 1000000 1000000 | 1000000 |
| CTL | AFOD | PBS |
| | AFCC | |
| | AFCC | |
| 1000000 | 1000000 1000000 1000000 1000000 1000000 1000000 1000000 1000000 1000000 | 1000000 |

Plate 1

| column | column | column | column | column | column | column | column | column | column | column | column |
|---|---|---|---|---|---|---|---|---|---|---|---|
| 1 | 2 | 3 | 4 | 5 | 6 | 7 | 8 | 9 | 10 | 11 | 12 |
| row A | 10000000 | 10000000 | 10000000 | 10000000 | 10000000 | 10000000 | 10000000 | 10000000 | 10000000 | 10000000 | 10000000 |
| | | | | | | 10000000 | | | | | |
| row B | CTL | AFCC RAAS 4 | | | | | | | | | PBS |
| row C | | AFCC RDNA | | | | | | | | | |
| row D | | Ribavirin | | | | | | | | | |
| row E | 10000000 | 10000000 | 10000000 | 10000000 | 10000000 | 10000000 | 10000000 | 10000000 | 10000000 | 10000000 | 10000000 |
| | | | | | | 10000000 | | | | | |
| row F | | | | | | | | | | | |
| row G | | | | | | | | | | | |
| row H | | | | | | | | | | | |

Note:
CTL: 100% inhibition control;
PBS: 0% inhibition control.

### 2 Raw Data; 2.1 Raw Data of Cytotoxicity Assay

1a plate1

| | column | column | column | column | column | column | column | column | column | column | [symbol] | [symbol] |
|---|---|---|---|---|---|---|---|---|---|---|---|---|
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 11788 | 37829 | 76241 | 79783 | 86094 | 89352 | 86475 | 84132 | 79922 | 82317 | 78529 | 84888 |
| B | | | | | | | | | | | | |
| row | 10513 | 38733 | 73718 | 79841 | 90368 | 82949 | 84058 | 85256 | 86834 | 85378 | 81751 | 78143 |
| C | | | | | | | | | | | | |
| row | 11907 | 71546 | 83521 | 89104 | 91831 | 87528 | 88304 | 89908 | 89782 | 81452 | 87404 | 80906 |
| D | | | | | | | | | | | | |
| row | 10873 | 82130 | 82349 | 86032 | 91782 | 93224 | 90052 | 88416 | 85029 | 87835 | 82113 | 80129 |
| E | | | | | | | | | | | | |
| row | 12015 | 61801 | 82574 | 79316 | 91001 | 90159 | 94232 | 93293 | 90416 | 91764 | 85286 | 75439 |
| F | | | | | | | | | | | | |
| row | 10586 | 51803 | 75949 | 84140 | 89954 | 84298 | 85969 | 87016 | 87714 | 84677 | 81008 | 81025 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

[symbol]

indicates data missing or illegible when filed

1a plate2

| | column | column | column | column | column | column | column | column | column | column | [symbol] | [symbol] |
|---|---|---|---|---|---|---|---|---|---|---|---|---|
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 12214 | 59805 | 68928 | 67269 | 68991 | 70963 | 70986 | 72721 | 80578 | 72648 | 86545 | 75138 |
| B | | | | | | | | | | | | |
| row | 10586 | 56271 | 62901 | 69768 | 63586 | 63753 | 61014 | 64486 | 70765 | 74224 | 84881 | 74471 |
| C | | | | | | | | | | | | |
| row | 12167 | 75390 | 86019 | 93902 | 94512 | 84075 | 78058 | 81619 | 78419 | 81311 | 81604 | 83171 |
| D | | | | | | | | | | | | |
| row | 10838 | 79348 | 85248 | 88417 | 90128 | 90987 | 89205 | 87054 | 80379 | 82754 | 79328 | 84199 |
| E | | | | | | | | | | | | |
| row | 12006 | 42127 | 56876 | 55340 | 70676 | 73336 | 84894 | 85941 | 87587 | 91910 | 91748 | 79542 |
| F | | | | | | | | | | | | |
| row | 10398 | 52814 | 54925 | 59760 | 72108 | 85112 | 88015 | 84100 | 88429 | 87978 | 88712 | 79154 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

[symbol]

indicates data missing or illegible when filed

1b plate1

| | column | column | column | column | column | column | column | column | column | column | [symbol] | [symbol] |
|---|---|---|---|---|---|---|---|---|---|---|---|---|
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 11869 | 51104 | 57291 | 60533 | 71572 | 71590 | 72696 | 63905 | 67104 | 64951 | 63293 | 68405 |
| B | | | | | | | | | | | | |
| row | 10705 | 46415 | 52869 | 63478 | 66044 | 76232 | 75102 | 64901 | 70704 | 64733 | 73663 | 65869 |
| C | | | | | | | | | | | | |
| row | 11915 | 48782 | 62222 | 70988 | 70061 | 72337 | 70822 | 62570 | 61489 | 69424 | 67863 | 62024 |
| D | | | | | | | | | | | | |
| row | 10698 | 54787 | 67780 | 74332 | 77817 | 76266 | 71439 | 69920 | 69209 | 68573 | 71055 | 76183 |
| E | | | | | | | | | | | | |
| row | 11617 | 56776 | 72151 | 78099 | 73707 | 80133 | 77881 | 71345 | 74569 | 75191 | 72729 | 67333 |
| F | | | | | | | | | | | | |
| row | 10389 | 55289 | 73692 | 79149 | 72098 | 79174 | 80854 | 75314 | 79363 | 74574 | 69452 | 70933 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

[symbol]

indicates data missing or illegible when filed

1b plate2

| | column | column | column | column | column | column | column | column | column | column | column | column |
|---|---|---|---|---|---|---|---|---|---|---|---|---|
| | 1 | 2 | 3 | 4 | 5 | 6 | 7 | 8 | 9 | 10 | 11 | 12 |
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 11781 | 46220 | 70386 | 71631 | 74038 | 70501 | 65402 | 59277 | 57714 | 59416 | 60015 | 55776 |
| B | | | | | | | | | | | | |
| row | 10659 | 50913 | 64365 | 63452 | 70091 | 66277 | 69189 | 64968 | 68110 | 71646 | 58898 | 58925 |
| C | | | | | | | | | | | | |
| row | 11590 | 53855 | 64467 | 71054 | 70043 | 66523 | 60263 | 62948 | 64591 | 67881 | 69418 | 52688 |
| D | | | | | | | | | | | | |
| row | 10463 | 59788 | 63077 | 66840 | 65994 | 75550 | 68223 | 63481 | 63360 | 64326 | 61607 | 64260 |
| E | | | | | | | | | | | | |
| row | 11215 | 31282 | 37580 | 38330 | 48594 | 59252 | 61156 | 62875 | 64284 | 66814 | 68225 | 60286 |
| F | | | | | | | | | | | | |
| row | 10340 | 34855 | 36605 | 40247 | 43076 | 56209 | 64876 | 65543 | 66800 | 66567 | 66665 | 64220 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

2a plate1

| | column | column | column | column | column | column | column | column | column | column | [symbol] | [symbol] |
|---|---|---|---|---|---|---|---|---|---|---|---|---|
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 11260 | 62423 | 63994 | 60008 | 66320 | 63246 | 63076 | 62824 | 54226 | 52388 | 56680 | 52388 |
| B | | | | | | | | | | | | |
| row | 10127 | 54433 | 51255 | 51497 | 56262 | 59280 | 55890 | 60222 | 55138 | 56626 | 57542 | 56626 |
| C | | | | | | | | | | | | |
| row | 11453 | 52361 | 58693 | 62869 | 69429 | 56045 | 58716 | 58284 | 60293 | 63778 | 58197 | 63778 |
| D | | | | | | | | | | | | |
| row | 10728 | 56908 | 65547 | 67010 | 64930 | 63451 | 64533 | 63630 | 63273 | 64781 | 64208 | 64781 |
| E | | | | | | | | | | | | |
| row | 11424 | 50095 | 64112 | 61153 | 63665 | 60082 | 61140 | 62072 | 66478 | 68446 | 61890 | 58446 |
| F | | | | | | | | | | | | |
| row | 10165 | 52406 | 60200 | 68101 | 64203 | 63244 | 61168 | 64479 | 63711 | 64375 | 61306 | 64375 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

[symbol]

indicates data missing or illegible when filed

2a plate2

| | column | column | column | column | column | column | column | column | column | column | [symbol] | [symbol] |
|---|---|---|---|---|---|---|---|---|---|---|---|---|
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 12001 | 66898 | 61275 | 60453 | 63884 | 61264 | 60534 | 60138 | 56469 | 62475 | 68167 | 66469 |
| B | | | | | | | | | | | | |
| row | 10936 | 66043 | 60181 | 55762 | 59218 | 56456 | 64653 | 56607 | 61353 | 60143 | 56251 | 61353 |
| C | | | | | | | | | | | | |
| row | 11751 | 60500 | 56343 | 66462 | 64470 | 62106 | 63364 | 56872 | 65881 | 62280 | 60706 | 65881 |
| D | | | | | | | | | | | | |
| row | 10693 | 64056 | 68127 | 69773 | 60913 | 69648 | 67707 | 67359 | 65950 | 64531 | 66975 | 65950 |
| E | | | | | | | | | | | | |
| row | 11776 | 37011 | 43034 | 47350 | 54734 | 60176 | 68095 | 70369 | 68319 | 70444 | 70185 | 68319 |
| F | | | | | | | | | | | | |
| row | 10164 | 38973 | 42537 | 43897 | 53024 | 59597 | 67739 | 65084 | 65506 | 65173 | 69459 | 65506 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

[symbol]

indicates data missing or illegible when filed

### 2.2 Raw Data of Anti-Replicon Activity Assay

1a plate1

| | column | column | column | column | column | column | column | column | column | column | [symbol] | [symbol] |
|---|---|---|---|---|---|---|---|---|---|---|---|---|
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 8 | 732 | 3768 | 3796 | 4068 | 4308 | 3768 | 3932 | 3632 | 3408 | 3640 | 3692 |
| B | | | | | | | | | | | | |
| row | 24 | 1060 | 3388 | 4176 | 3904 | 3672 | 3896 | 3340 | 3132 | 3468 | 3248 | 3236 |
| C | | | | | | | | | | | | |
| row | 28 | 3172 | 3916 | 4364 | 4156 | 3660 | 3384 | 3312 | 3516 | 3380 | 3336 | 3684 |
| D | | | | | | | | | | | | |
| row | 32 | 3736 | 4300 | 4028 | 4428 | 3840 | 3904 | 3668 | 3828 | 3852 | 3812 | 3804 |
| E | | | | | | | | | | | | |
| row | 20 | 2120 | 4036 | 4316 | 4452 | 4276 | 3728 | 3708 | 4092 | 3676 | 3656 | 4148 |
| F | | | | | | | | | | | | |
| row | 28 | 2040 | 4080 | 4044 | 4156 | 4316 | 4084 | 4008 | 3912 | 3992 | 4028 | 3844 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

[symbol]

indicates data missing or illegible when filed

1a plate2

| | column | column | column | column | column | column | column | column | column | column | column | column |
|---|---|---|---|---|---|---|---|---|---|---|---|---|
| | 1 | 2 | 3 | 4 | 5 | 6 | 7 | 8 | 9 | 10 | 11 | 12 |
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 24 | 3312 | 4300 | 3624 | 4348 | 3636 | 3692 | 3756 | 3188 | 3612 | 3488 | 3396 |
| B | | | | | | | | | | | | |
| row | 28 | 3552 | 4168 | 3480 | 4268 | 3692 | 3580 | 3592 | 3832 | 3748 | 3384 | 3396 |
| C | | | | | | | | | | | | |
| row | 28 | 3792 | 4188 | 4276 | 4504 | 3768 | 4292 | 3688 | 3452 | 3676 | 3600 | 3720 |
| D | | | | | | | | | | | | |
| row | 20 | 4112 | 4396 | 4180 | 4104 | 3800 | 3884 | 3868 | 3936 | 3332 | 3396 | 3392 |
| E | | | | | | | | | | | | |
| row | 36 | 116 | 728 | 1608 | 2804 | 3524 | 4012 | 4076 | 4232 | 3760 | 3856 | 4032 |
| F | | | | | | | | | | | | |
| row | 12 | 52 | 196 | 1088 | 2800 | 3880 | 4000 | 4284 | 4360 | 4152 | 3912 | 4188 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

1b plate1

| | column | column | column | column | column | column | column | column | column | column | [symbol] | [symbol] |
|---|---|---|---|---|---|---|---|---|---|---|---|---|
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 24 | 3416 | 3652 | 3304 | 3688 | 3620 | 3400 | 3400 | 3348 | 3048 | 3096 | 3388 |
| B | | | | | | | | | | | | |
| row | 28 | 3464 | 3236 | 3852 | 3236 | 3400 | 3760 | 3316 | 3216 | 3048 | 3020 | 3338 |
| C | | | | | | | | | | | | |
| row | 24 | 2968 | 3176 | 3476 | 2956 | 3324 | 3440 | 3196 | 2748 | 2628 | 3108 | 3524 |
| D | | | | | | | | | | | | |
| row | 40 | 3180 | 2932 | 3408 | 3176 | 3696 | 3264 | 2912 | 3480 | 2768 | 2776 | 3596 |
| E | | | | | | | | | | | | |
| row | 28 | 3132 | 3760 | 3932 | 3724 | 3548 | 3452 | 3968 | 3172 | 3196 | 3228 | 3740 |
| F | | | | | | | | | | | | |
| row | 20 | 3248 | 3976 | 3888 | 3836 | 4060 | 3484 | 3440 | 3328 | 3028 | 3124 | 3496 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

[symbol]

indicates data missing or illegible when filed

1b plate2

| | column | column | column | column | column | column | column | column | column | column | [symbol] | [symbol] |
|---|---|---|---|---|---|---|---|---|---|---|---|---|
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 20 | 3788 | 3852 | 3664 | 3728 | 3944 | 3584 | 3436 | 3192 | 3348 | 3740 | 3588 |
| B | | | | | | | | | | | | |
| row | 36 | 3548 | 3964 | 3416 | 3352 | 3280 | 3232 | 3188 | 3200 | 3052 | 3064 | 3576 |
| C | | | | | | | | | | | | |
| row | 32 | 3856 | 3876 | 4044 | 3428 | 3364 | 3876 | 3600 | 3080 | 3496 | 3356 | 3624 |
| D | | | | | | | | | | | | |
| row | 24 | 4048 | 4036 | 3980 | 3924 | 3328 | 3704 | 3780 | 3388 | 3312 | 3504 | 3880 |
| E | | | | | | | | | | | | |
| row | 24 | 36 | 172 | 680 | 1548 | 3368 | 3596 | 3820 | 3708 | 3724 | 3760 | 4340 |
| F | | | | | | | | | | | | |
| row | 16 | 32 | 232 | 752 | 2116 | 3372 | 3668 | 4032 | 4116 | 3852 | 4208 | 4096 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

[symbol]

indicates data missing or illegible when filed

2a plate1

| | column | column | column | column | column | column | column | column | column | column | [symbol] | [symbol] |
|---|---|---|---|---|---|---|---|---|---|---|---|---|
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 24 | 2844 | 2960 | 2856 | 2412 | 2644 | 2548 | 2388 | 2388 | 2304 | 2564 | 2352 |
| B | | | | | | | | | | | | |
| row | 32 | 3172 | 2856 | 2708 | 2652 | 2388 | 2200 | 2428 | 2056 | 2444 | 2328 | 2224 |
| C | | | | | | | | | | | | |
| row | 32 | 2136 | 2504 | 2360 | 2268 | 2108 | 2156 | 2248 | 2096 | 2304 | 2056 | 2492 |
| D | | | | | | | | | | | | |
| row | 20 | 2280 | 2720 | 2684 | 2260 | 2332 | 2244 | 2304 | 2572 | 2208 | 1888 | 2532 |
| E | | | | | | | | | | | | |
| row | 28 | 3068 | 2664 | 2908 | 2524 | 2804 | 3092 | 2484 | 2608 | 2380 | 2232 | 2416 |
| F | | | | | | | | | | | | |
| row | 16 | 2820 | 2984 | 3016 | 2892 | 2944 | 2956 | 2804 | 2392 | 2752 | 2628 | 3216 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

[symbol]

indicates data missing or illegible when filed

2a plate2

| | column | column | column | column | column | column | column | column | column | column | [symbol] | [symbol] |
|---|---|---|---|---|---|---|---|---|---|---|---|---|
| row | 10000000 | 10000000 | 10000000 | 10000000 | 10000000 | 10000000 | 10000000 | 10000000 | 10000000 | 10000000 | 10000000 | 10000000 |
| A | | | | | | | | | | | | |
| row | 20 | 2700 | 2812 | 2628 | 2572 | 2624 | 2604 | 2460 | 2460 | 2484 | 2456 | 2696 |
| B | | | | | | | | | | | | |
| row | 28 | 2752 | 2768 | 2416 | 2208 | 2804 | 2440 | 2188 | 2884 | 2204 | 2240 | 2548 |
| C | | | | | | | | | | | | |
| row | 24 | 2508 | 3016 | 2568 | 2580 | 2744 | 2064 | 2504 | 2288 | 2084 | 2168 | 2504 |
| D | | | | | | | | | | | | |
| row | 36 | 2676 | 2740 | 2740 | 2404 | 2536 | 2632 | 2236 | 2016 | 2408 | 2228 | 2232 |
| E | | | | | | | | | | | | |
| row | 28 | 56 | 184 | 548 | 1024 | 1428 | 2436 | 2276 | 2348 | 2468 | 2512 | 2692 |
| F | | | | | | | | | | | | |
| row | 20 | 48 | 200 | 688 | 1396 | 1856 | 2248 | 2712 | 2532 | 2284 | 2520 | 2820 |
| G | | | | | | | | | | | | |
| row | 10000000 | 10000000 | 10000000 | 10000000 | 10000000 | 10000000 | 10000000 | 10000000 | 10000000 | 10000000 | 10000000 | 10000000 |
| H | | | | | | | | | | | | |

[symbol]

indicates data missing or illegible when filed

3 Cytotoxicity and anti-replicon activity of the human plasma derived proteins.

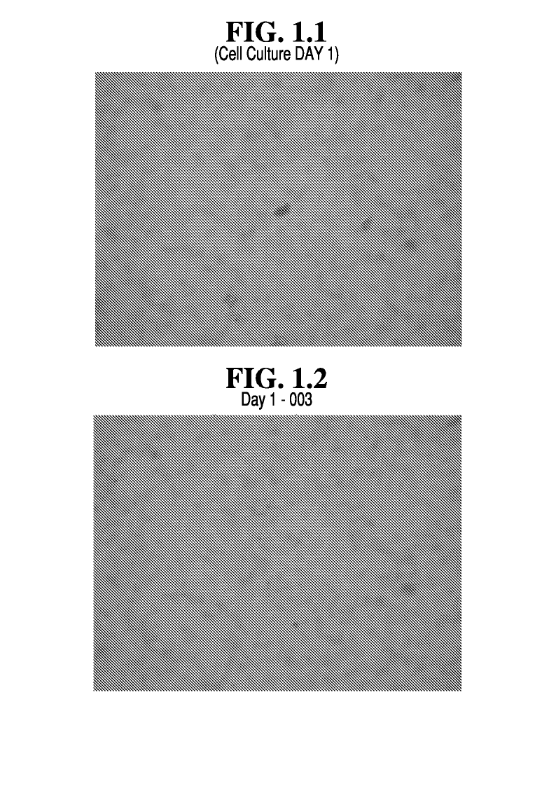

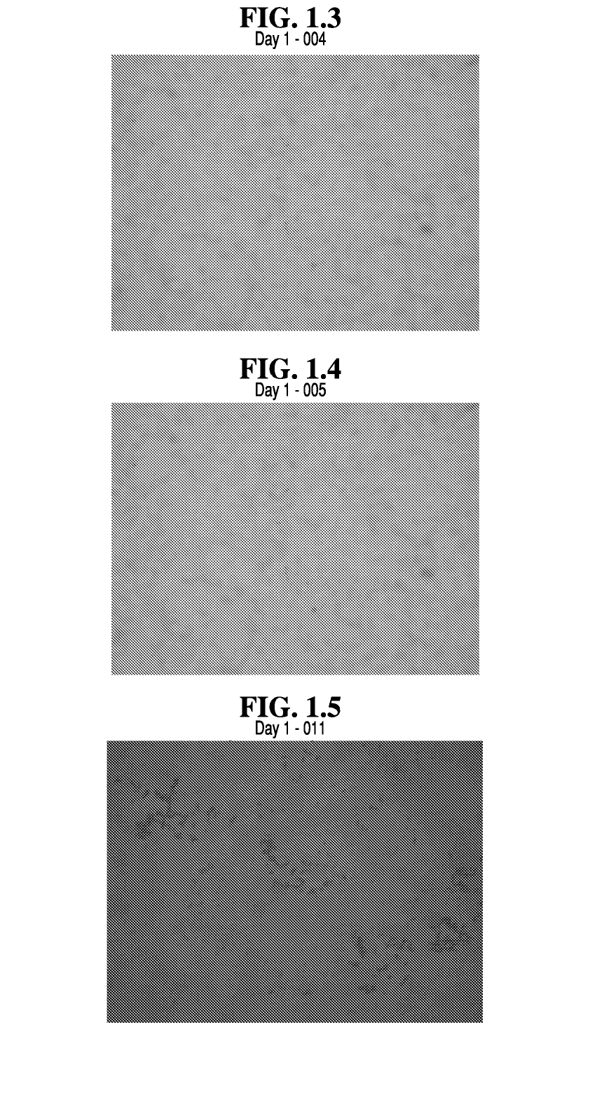

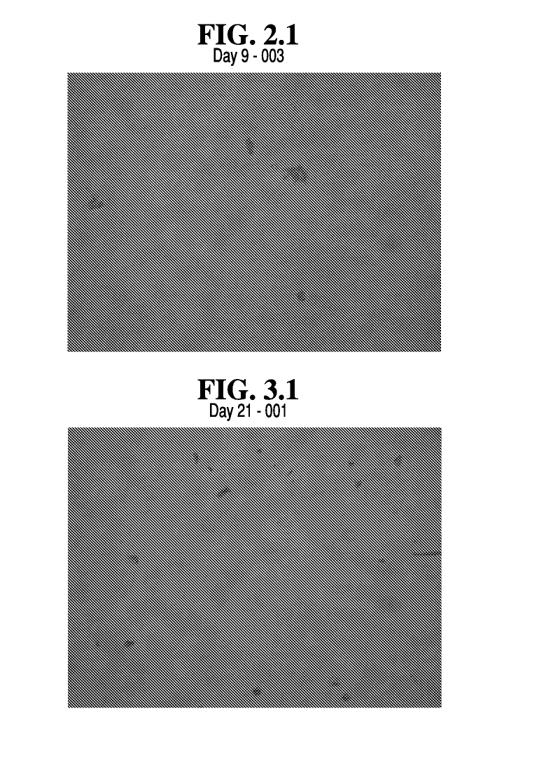

CC50 and EC50 values are summarized in Table 4. GraphPad Prism files containing dose-dependent curves are presented in this report. CC50 and EC50 values are shown in FIG. 1 and FIG. 2 respectively.

TABLE 4
CC₅₀ and EC₅₀ Summary of the human plasma derived proteins

| | 1a | | 1b | | 2a | |
|---|---|---|---|---|---|---|
| Name | CC₅₀ (ug/ml) | EC₅₀ (ug/ml) | CC₅₀ (ug/ml) | EC₅₀ (ug/ml) | CC₅₀ (ug/ml) | EC₅₀ (ug/ml) |
| AFOD KH | 60.7% inh at | 76.5% inh at | >400 | >400 | >400 | >400 |
| | 400 ug/ml | 400 ug/ml | | | | |
| AFCC KH | >400 | >400 | >400 | >400 | >400 | >400 |
| AFCC RAAS 1 | 33.8% inh at | 44.5% inh at | >400 | >400 | >400 | >400 |
| | 400 ug/ml | 400 ug/ml | | | | |
| AFCC RAAS 4 | >400 | >400 | >400 | >400 | >400 | >400 |
| AFCC RDNA | >400 | >400 | >400 | >400 | >400 | >400 |
| | CC₅₀ (uM) | EC₅₀ (uM) | CC₅₀ (uM) | EC₅₀ (uM) | CC₅₀ (uM) | EC₅₀ (uM) |
| Ribavirin | 47.4% inh at | 57.58 | 61.8% inh at | 39.04 | 44.8% inh at | 37.44 |
| | 320 Um | | 320 uM | | 320 uM | |

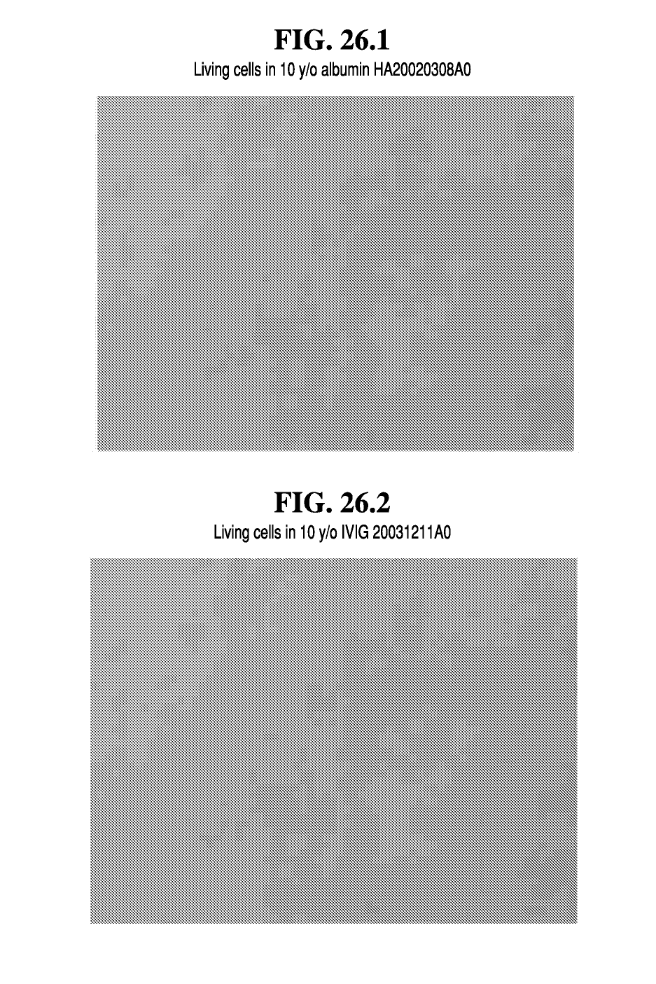

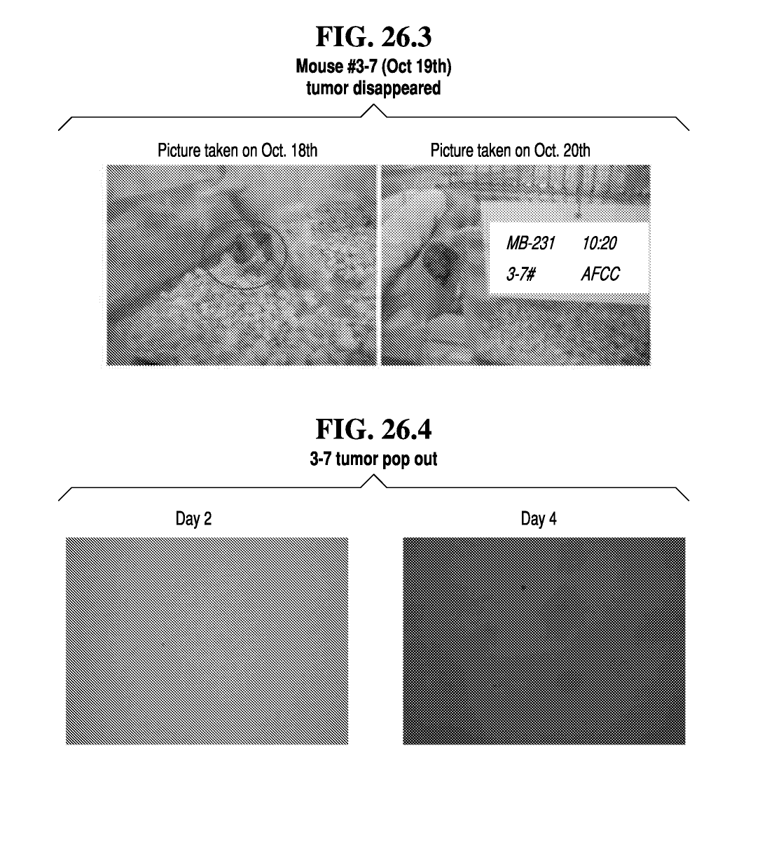

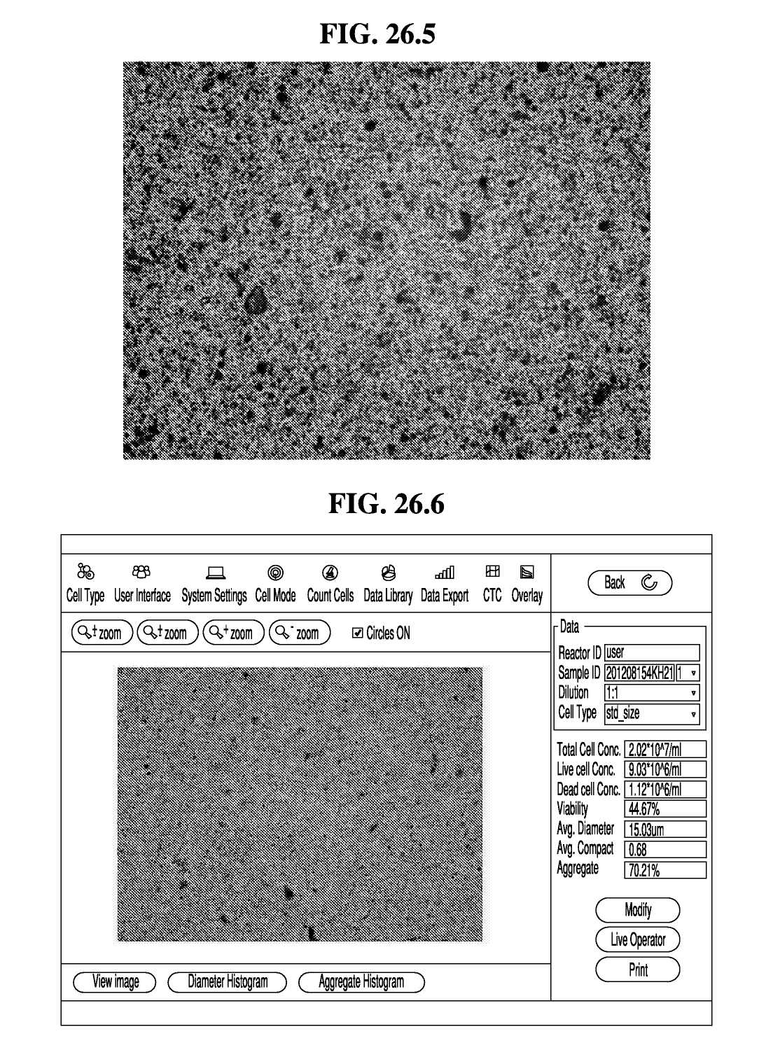

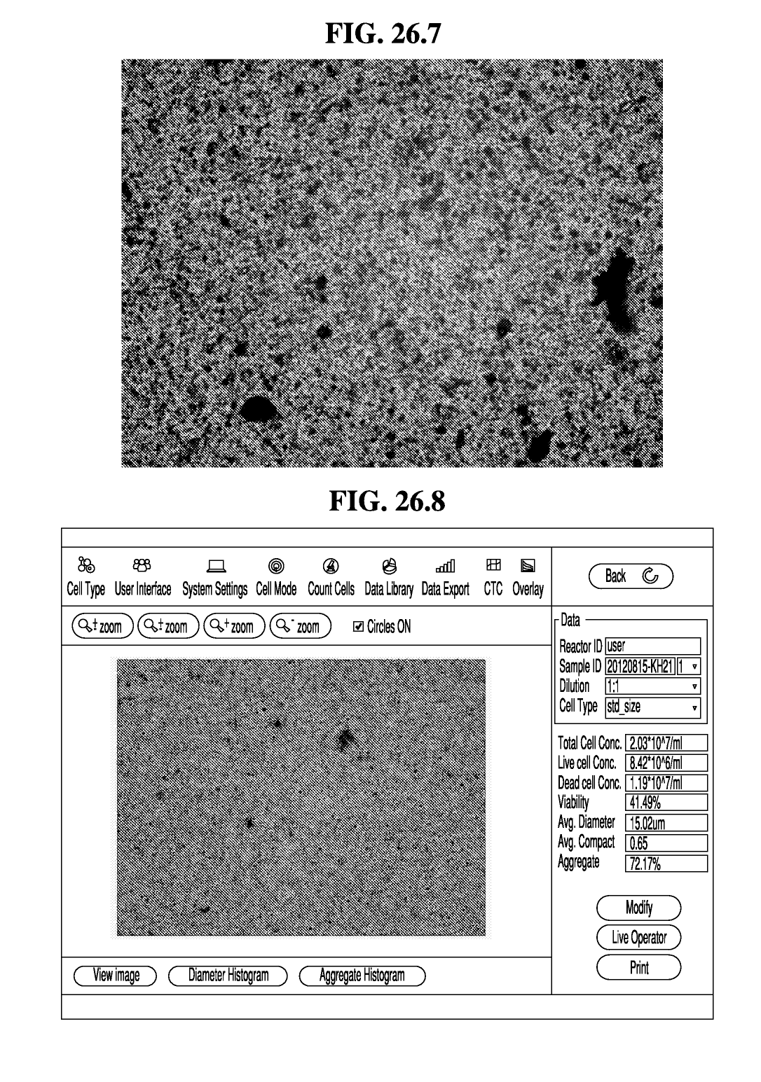

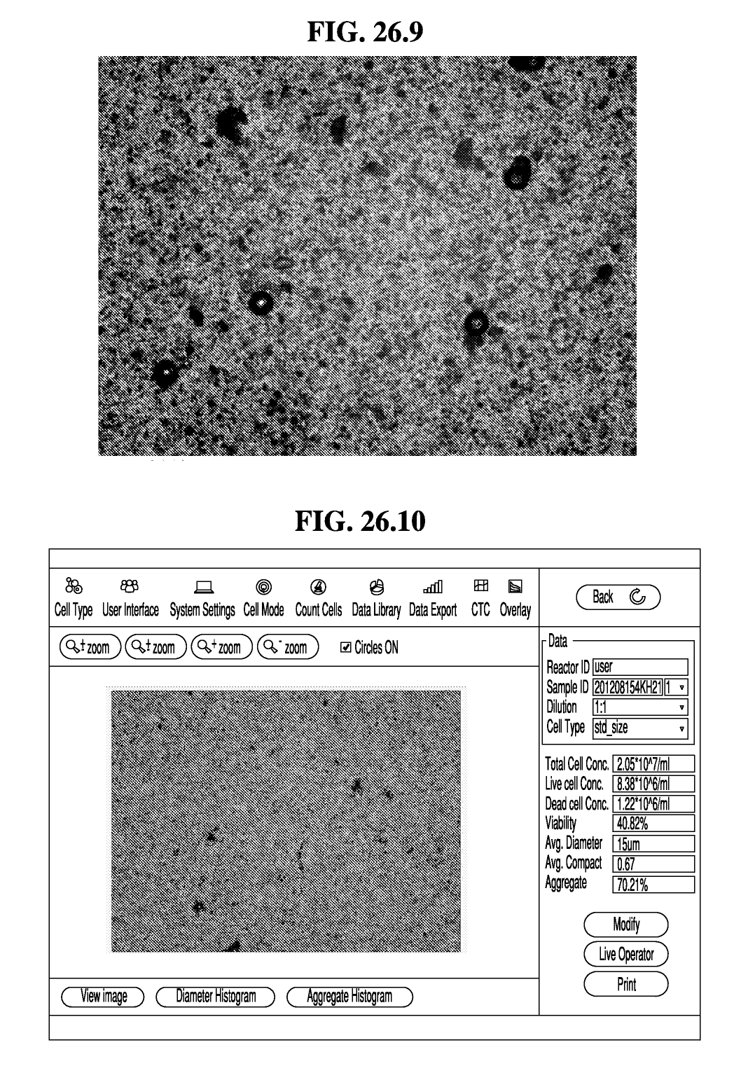

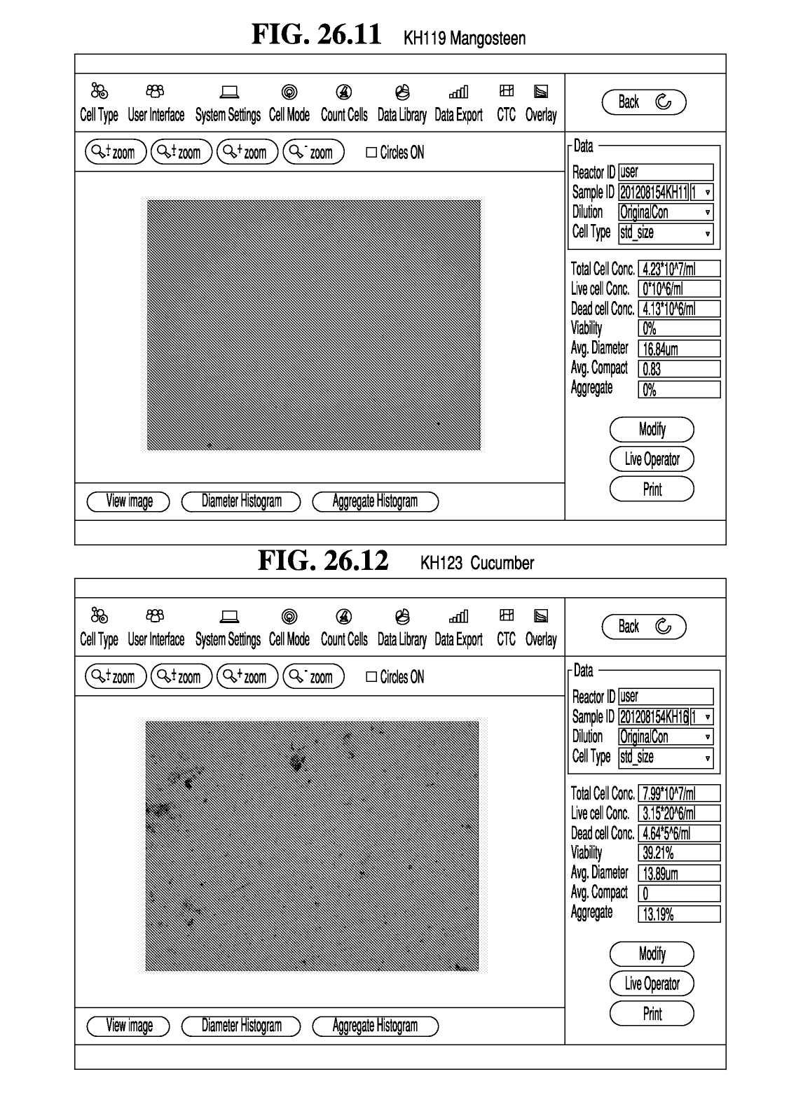

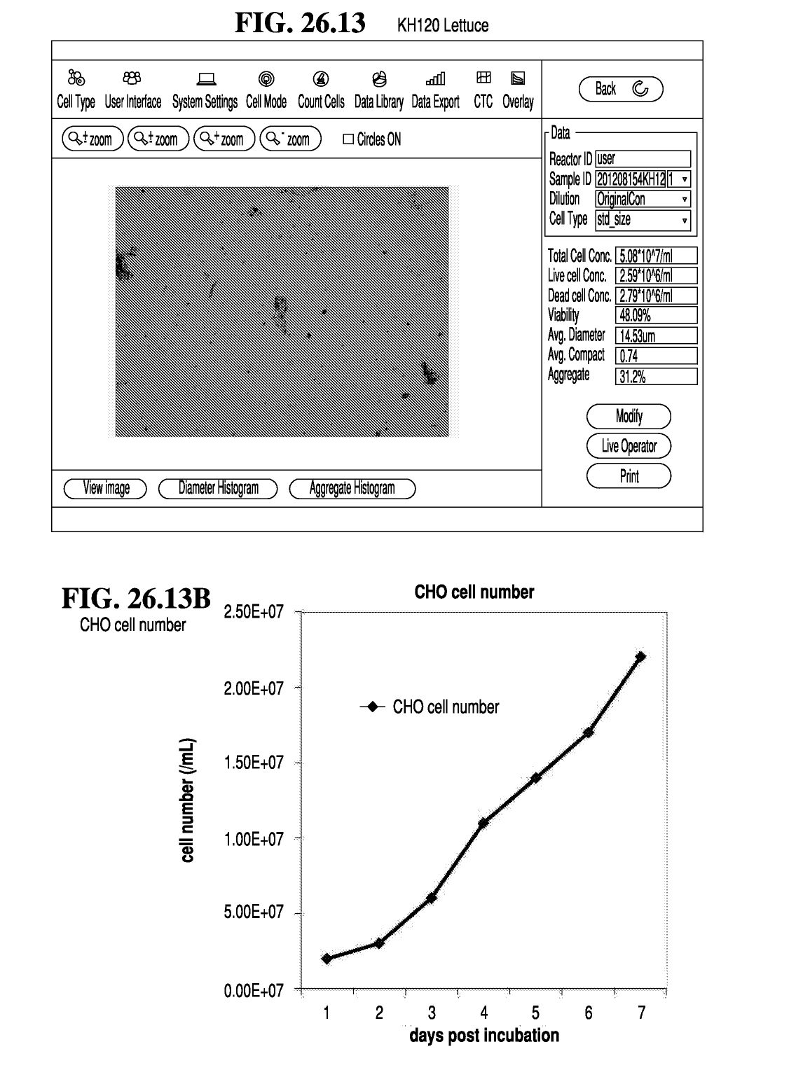

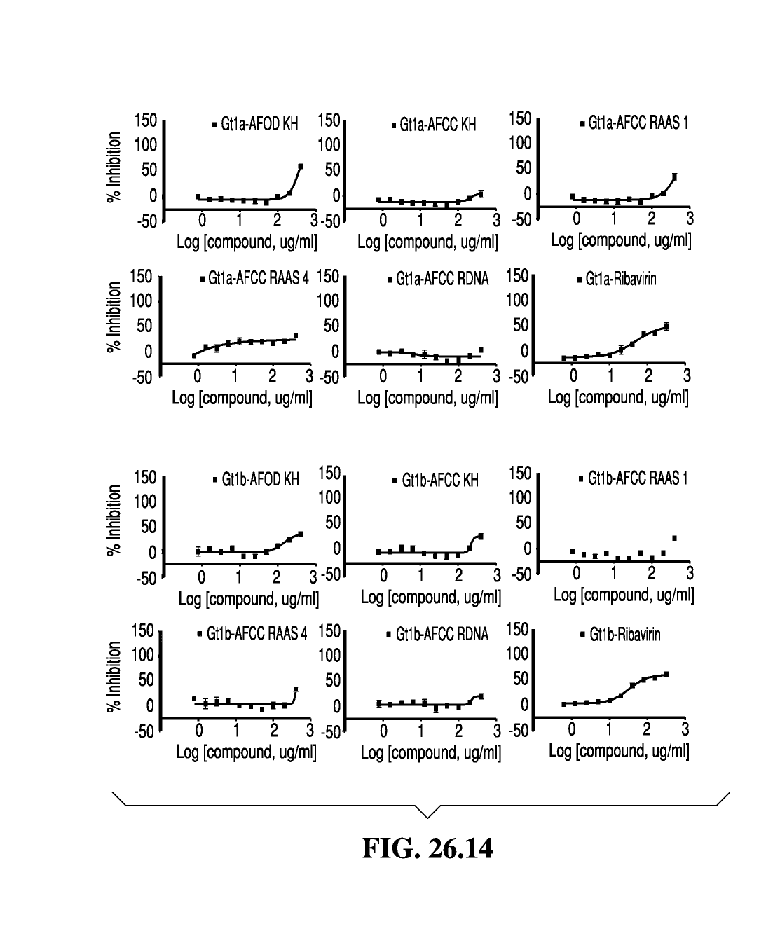

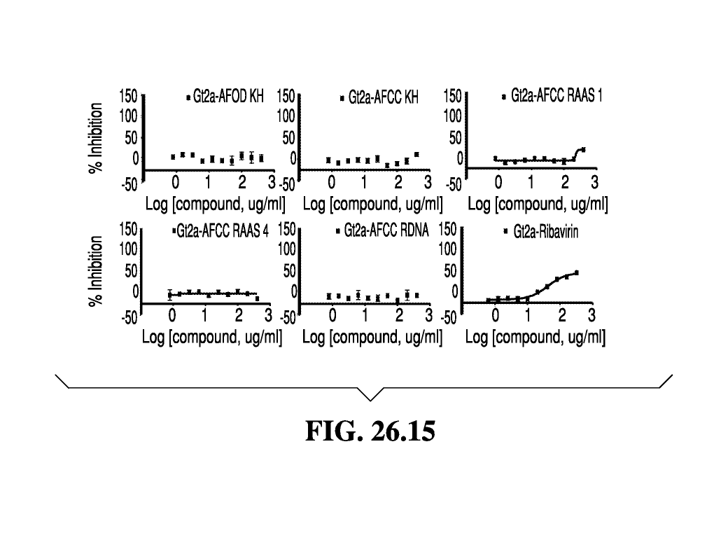

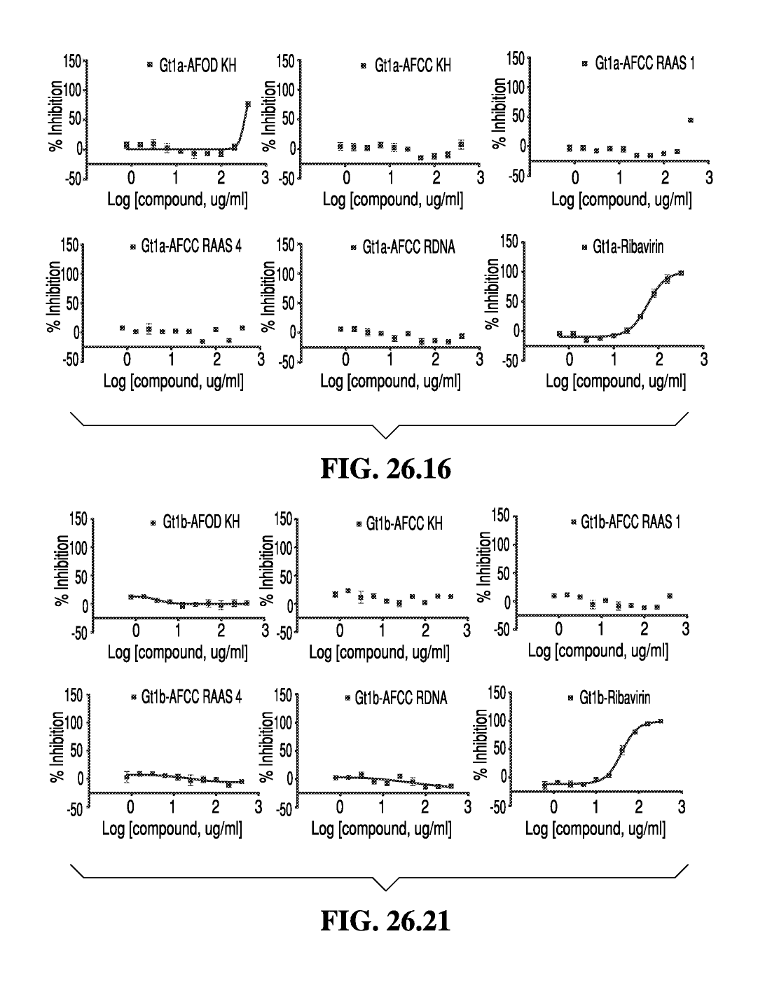

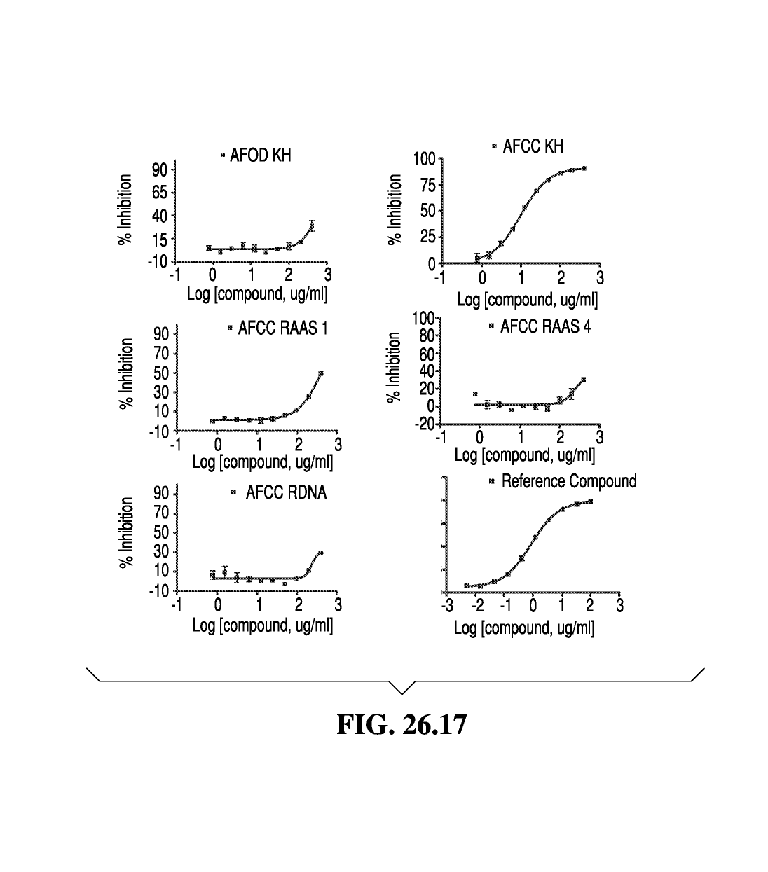

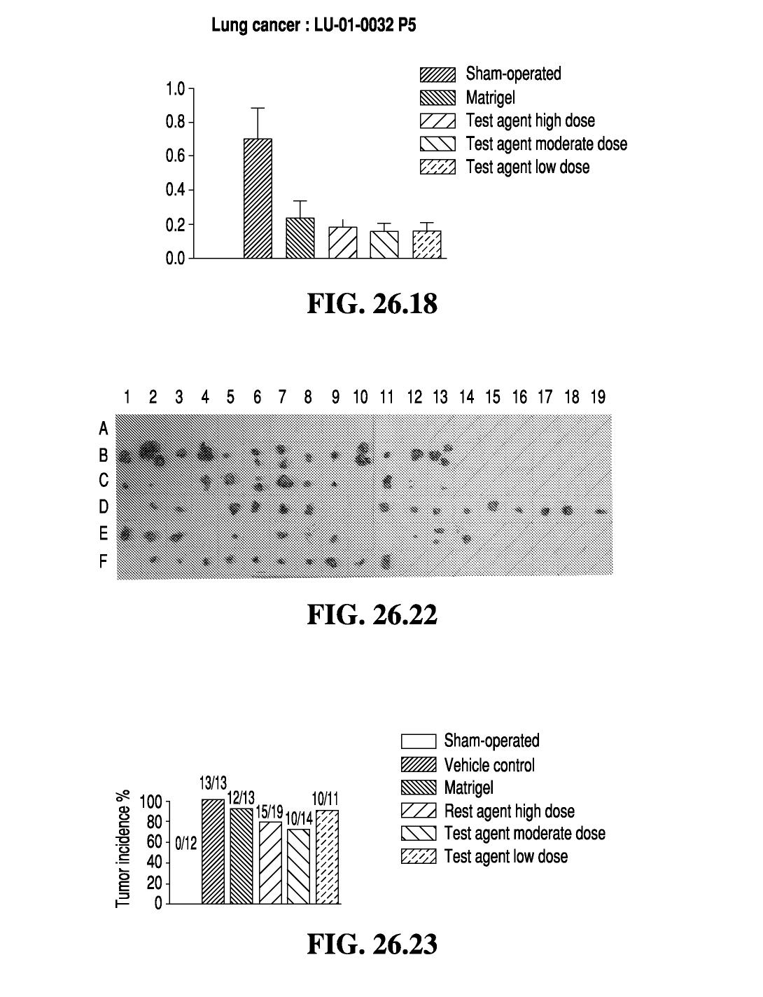

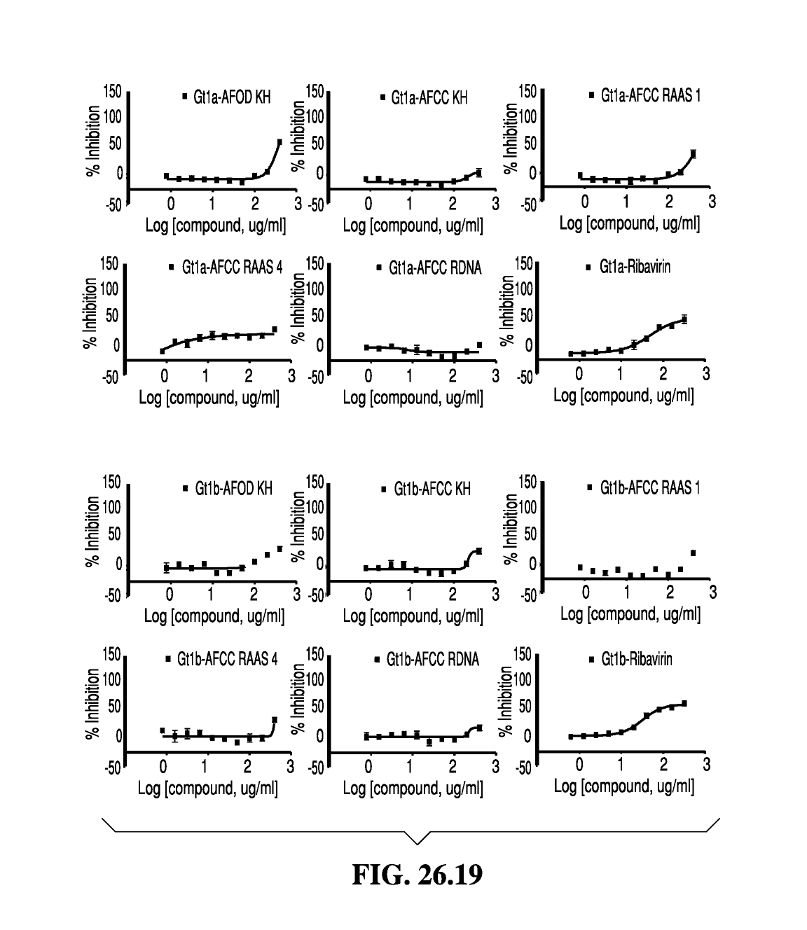

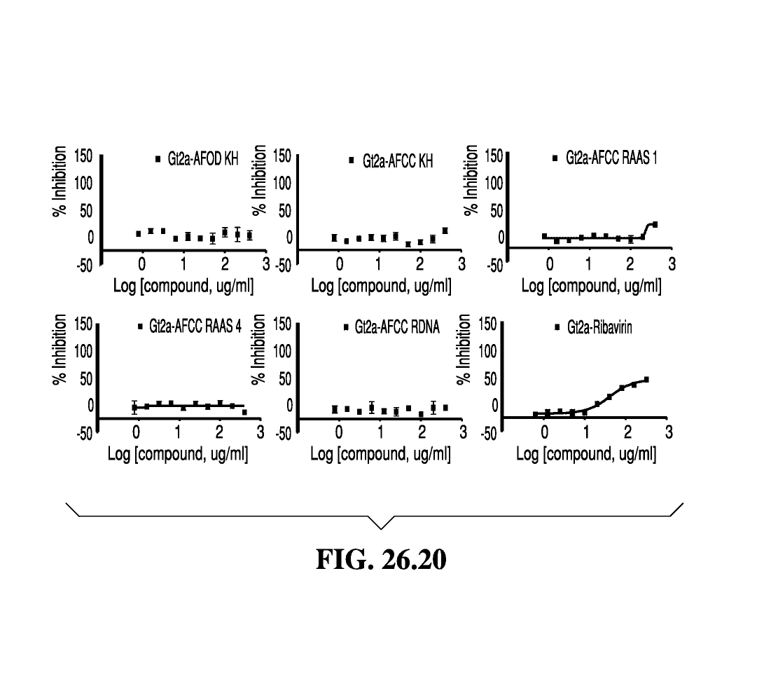

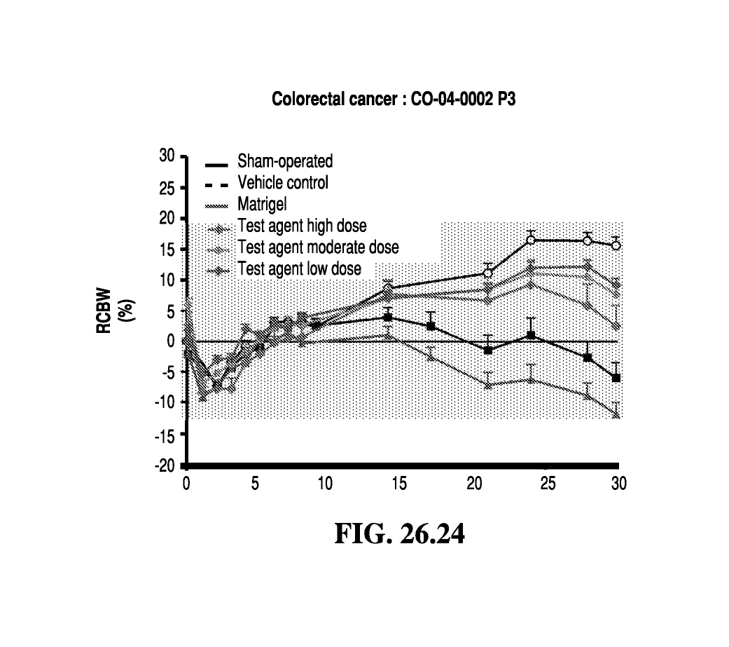

The following figure designations, such as FIGS. 26.14, 16.15, refer to figures of Group A, a first group of figures in the present application. A second group of figures in the present application, Group B, which will be referred to later in the application, will contain some figures that have the same designation as figures of Group A. See FIGS. 26.14, 16.15. Dose-dependent curves (CC50 values) and FIG. 26.19, 16.20. Dose-dependent curves (EC50 values)

### IV. Conclusions

The Z factors of the cytotoxicity assay plates are 0.83(1a-plate1), 0.79 (1a-plate2),
   0.71(1b-plate1), 0.68 (1b-plate2), 0.65 (2a-plate1) and 0.83(2a-palte2), which are better than our QC standard.
The Z factors of the anti-replicon assay plates are 0.75(1a-plate1), 0.70 (1a-plate2),
   0.87 (1b-plate1), 0.75 (1b-plate2), 0.58 (2a-plate1) and 0.75(2a-palte2), which are better than our QC standard.
EC₅₀ of the positive control Ribavirin in this study are 57.58 uM (1a), 39.04 uM (1b), and
   37.44 (2a), which are consistent with our previous data.

### V. References

1. Mutations in Hepatitis C Virus RNAs Conferring Cell Culture Adaptation V. Lohmann et al., 2001 J. Virol.
2. Development of a replicon-based phenotypic assay for assessing the drug susceptibilities of HCV NS3 protease genes from clinical isolates. Qi X et al., Antiviral Res. 2009 February; 81(2:)166

### I. Study Objective

INFLUENZA STUDYTo Test 2 Compounds from RAAS for Anti-Influenza Activity Against Strains A/Weiss/43

### H1N1 in cell culture

### II. Study Protocols

### 3. Materials:

Cell Line: MDCK cells

### 1.2 Compounds:

The test articles are supplied in the form of dry powder or 10 mM solution, and Oseltamivir as control, in duplicate.

### 1.3 Reagents:

The following table designations, such as Table 5.1, refer to tables of a first group of tables in the present application. Other groups of tables in the present application, which will be referred to later in the application, will contain some tables that have the same designations as tables of the first group.

TABLE 5.1
List of reagents and consumable

| Reagent | Vendor | Catalog Number |
|---|---|---|
| Dimethyl sulfoxide (DMSO) | Sigma | Cat#D8418 |
| SFM | Invitrogen | Cat# 12309-019 |
| Fetal Bovine Serum (FBS) | Gibco | Cat#16140 |
| Penicillin-Streptomycin | Invitrogen | Cat# 15140-122 |
| MEM non-essential amino acids | Invitrogen | cat# 11140-076 |
| GlutaMAX-I Supplement | Invitrogen | Cat# 35050-061 |
| Trypsin/EDTA | Invitrogen | Cat# 25300-062 |
| PBS | Invitrogen | Cat#10010-049 |
| DPBS/Modified | Hyclone | SH30028.01B |
| 96 well cell plate | Corning | Cat#3599 |
| MTT | sigma | Cat# M2128 |

### 1.4 Instrument

speterphotemeter (Molecular Devices)
Multidrop (Thermo)
Janus (perkinelmer)

### 4. Methods

### 2.1 Cell Addition

T150 flask containing MDCK cell monolayer is rinsed with 10 ml pre-warmed PBS. Add 3 ml of pre-warmed Trypsin 0.25% and incubate at 5% CO₂, 37° C. for 3 minutes. Nine milliliters of DMEM complete media are added, and the cells are blown for 30 s by pipetting. The cells are counted using hemocytometer.

MDCK cells are resuspended in SFM medium to reach a cell density of 50,000 cells/ml (to obtain a final cell plating density of 5000 cells/100 ul/well). Plate cells in 96 well plate using Multidrop. Incubate plate at 5% CO₂, 37° C. for overnight.

### 2.2 Compound Addition

RAAS provided the test articles in the form of dry powder or liquid (Table 5.2). Test samples were diluted in PBS as 3.5×10⁴ m/ml stocks. Sample dilutions are made by Janus with 2-fold serial dilutions for 8 concentrations plus PBS. Osletamivir is diluted with 3-fold for 8 concentrations. The final sample concentrations of the anti-influenza assay are described in Table 5.3.

TABLE 5.2
Sample information

| Name | Protein conc. | Formulation | Diluents |
|---|---|---|---|
| AFOD KH | 10% | Liquid | |
| AFCC KH | 3.50% | Liquid | |
| AFCC RAAS 1 | 4% | Lyophilized | AFOD KH 10 mL |
| AFCC RAAS 4 | 0.0020% | Lyophilized | AFOD KH 10 mL |
| AFCC RDNA | 0.00001% | Lyophilized | AFOD KH 10 mL |

TABLE 5.3
Sample or compound concentrations for EC₅₀ and CC₅₀ measurement

Name

| | Concentration (μg/ml) | | | | | | | |
|---|---|---|---|---|---|---|---|---|
| AFOD KH | 400 | 200 | 100 | 50 | 25 | 12.5 | 6.3 | 3.1 |
| AFCC KH | 400 | 200 | 100 | 50 | 25 | 12.5 | 6.3 | 3.1 |
| AFCC RAAS 1 | 400 | 200 | 100 | 50 | 25 | 12.5 | 6.3 | 3.1 |
| AFCC RAAS 4 | 400 | 200 | 100 | 50 | 25 | 12.5 | 6.3 | 3.1 |
| AFCC RDNA | 400 | 200 | 100 | 50 | 25 | 12.5 | 6.3 | 3.1 |
| | Concentration (μM) | | | | | | | |
| Osletamivir | 100.00 | 33.33 | 11.11 | 3.70 | 1.23 | 0.41 | 0.14 | 0.05 |

2.3 Detection (after 72 Hours of Incubation)

MTT solution is prepared freshly. Plates are removed from incubator to allow equilibration to room temperature. Multidrop is used to add 20 ul MTT to each well of compound-treated cells. The plates are incubated for 4 hour, and then read on a speterphotemeter for EC50 and cytotoxicity calculation.

The anti-influenza activity (% inhibition) is calculated using the equation below

The cytotoxicity is calculates using the equation below:

% livability=(Cmpd/PBS control)*100

Dose-response curves are plotted using Prism.

### III. Assay Results

### 1 Assay Plate Map

### For Anti-Influenza Activity

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | |
|---|---|---|---|
| A | | | |
| B | cpd1 | VC | CC |
| C | | | |
| D | cpd2 | VC | CC |
| E | | | |
| F | cpd3 | VC | CC |
| G | | | |
| H | | | |

| | | | | | | | |
|---|---|---|---|---|---|---|---|
| 400.000 | 200.000 | 100.000 | 50.000 | 25.000 | 12.500 | 6.250 | 3.125 |

Doses (ug/ml)

Note:
CC: 100% inhibition control;
VC: 0% inhibition control.
For cytotoxicity:

| | 1 | 2 | 3 | 4 | 5 | 6 | 7 | 8 | 9 | 10 | 11 | 12 |
|---|---|---|---|---|---|---|---|---|---|---|---|---|
| A | | | | | | | | | | | | |

| | | | |
|---|---|---|---|
| B | cpd1 | CC | CC |
| C | | | |
| D | cpd2 | CC | CC |
| E | | | |
| F | cpd3 | CC | CC |
| G | | | |
| H | | | |

| | | | | | | | |
|---|---|---|---|---|---|---|---|
| 400.000 | 200.000 | 100.000 | 50.000 | 25.000 | 12.500 | 6.250 | 3.125 |

Doses (ug/ml)

Note:
CC: 100% livability control.

### 2 Raw Data

### 2.1 Raw Data of Anti-Influenza Assay

plate1

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | | | |
|---|---|---|---|---|---|---|---|---|---|---|
| A B | | | | | | | | | | |
| | 0.935 | 1.478 | 1.435 | 0.247 | 0.221 | 0.212 | 0.188 | 0.193 | 0.136 | 1.504 |
| C | 1.032 | 1.345 | 1.276 | 0.455 | 0.241 | 0.226 | 0.203 | 0.188 | 0.216 | 1.439 |
| D | 1.348 | 1.308 | 1.375 | 1.485 | 0.221 | 0.171 | 0.197 | 0.158 | 0.159 | 1.506 |
| E | 1.362 | 1.429 | 1.466 | 1.386 | 0.234 | 0.159 | 0.173 | 0.208 | 0.167 | 1.565 |
| F | 1.486 | 1.318 | 0.963 | 0.264 | 0.173 | 0.173 | 0.185 | 0.181 | 0.163 | 1.477 |
| G H | 1.584 | 1.432 | 0.948 | 0.322 | 0.224 | 0.217 | 0.205 | 0.149 | 0.131 | 1.468 |

plate2

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | | | |
|---|---|---|---|---|---|---|---|---|---|---|
| A B | | | | | | | | | | |
| | 1.484 | 1.396 | 0.819 | 0.273 | 0.224 | 0.182 | 0.145 | 0.171 | 0.180 | 1.279 |
| C | 1.464 | 1.294 | 0.668 | 0.236 | 0.174 | 0.224 | 0.176 | 0.179 | 0.189 | 1.261 |
| D | 1.411 | 1.238 | 0.279 | 0.183 | 0.207 | 0.237 | 0.175 | 0.177 | 0.150 | 1.262 |
| E | 1.418 | 1.128 | 0.306 | 0.211 | 0.180 | 0.178 | 0.231 | 0.176 | 0.172 | 1.238 |
| F | 1.290 | 1.382 | 1.296 | 1.266 | 0.969 | 0.563 | 0.544 | 0.386 | 0.353 | 1.319 |
| G H | 1.292 | 1.218 | 1.210 | 1.295 | 0.962 | 0.627 | 0.431 | 0.388 | 0.394 | 1.397 |

### Raw Data of Cytotoxicity Assay

| plate1 | 1 | 2 | 3 | 4 | 5 | 6 | 7 | 8 | 9 | 10 | 11 | 12 |
|---|---|---|---|---|---|---|---|---|---|---|---|---|
| AB | | | | | | | | | | | | |
| | | 1.490 | 1.619 | 1.584 | 1.420 | 1.037 | 1.183 | 1.139 | 1.101 | 1.161 | 1.209 | |
| C | | 1.593 | 1.550 | 1.482 | 1.440 | 0.995 | 1.173 | 1.337 | 1.043 | 1.122 | 1.261 | |
| D | | 1.366 | 1.332 | 1.230 | 1.301 | 1.321 | 1.279 | 1.227 | 1.322 | 1.238 | 1.306 | |
| E | | 1.308 | 1.323 | 1.225 | 1.273 | 1.268 | 1.247 | 1.274 | 1.357 | 1.318 | 1.326 | |
| F | | 1.788 | 1.718 | 1.471 | 1.418 | 1.406 | 1.373 | 1.295 | 1.340 | 1.257 | 1.270 | |
| G | | 1.798 | 1.741 | 1.455 | 1.543 | 1.471 | 1.320 | 1.352 | 1.367 | 1.275 | 1.216 | |
| H | | | | | | | | | | | | |

plate2

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | | | |
|---|---|---|---|---|---|---|---|---|---|---|
| A | | | | | | | | | | |
| B | 1.793 | 1.799 | 1.852 | 1.776 | 1.796 | 1.639 | 1.626 | 1.650 | 1.626 | 1.524 |
| C | 1.842 | 1.870 | 1.818 | 1.939 | 1.773 | 1.690 | 1.631 | 1.649 | 1.675 | 1.564 |
| D | 1.822 | 1.897 | 1.849 | 1.891 | 1.688 | 1.689 | 1.641 | 1.637 | 1.713 | 1.617 |
| E | 1.830 | 1.944 | 1.913 | 1.874 | 1.812 | 1.606 | 1.630 | 1.652 | 1.605 | 1.570 |
| H | | | | | | | | | | |

### Cytotoxicity and Anti-Influenza Activity of the Human Plasma Derived Proteins.

CC₅₀ and EC₅₀ values are summarized in Table 5.4. GraphPad Prism files containing dose-dependent curves are presented in this report. CC₅₀ and EC₅₀ values are shown in FIG. 26.17 and FIG. 26.21 respectively.

TABLE 5.4
CC₅₀ and EC₅₀ Summary of the human plasma derived proteins

| cpds | anti H1N1 EC50s | CC50s (ug/ml) |
|---|---|---|
| AFOD KH | 69.06 | >400 |
| AFCC KH | 35.37 | >400 |
| AFCC RAAS 1 | 89.63 | >400 |
| AFCC RAAS 4 | 108.40 | >400 |
| AFCC RDNA | 154.90 | >400 |
| cpds | anti H1N1 EC50s (uM) | |
| Oseltamivir | 0.89 | |

### IV. Conclusions

The EC₅₀ of the positive control Osletamivir in this study is 0.89 uM, which is consistent with our previous data.
The human plasma derived proteins showed anti-influenza activity in this study.

### I. Study Objective

### HIV Study

To analyze human plasma derived proteins for anti-HIV activity on HIV-RT enzyme

### II. Study Protocols

### 5. Materials:

### 1.1 Samples Information:

RAAS provided the test articles in the form of dry powder or liquid (Table 6.1). Wuxi provided reference compound in DMSO solution.

TABLE 6.1
Sample information

| Name | Protein conc. | Formulation | Diluents |
|---|---|---|---|
| AFOD KH | 10% | Liquid | |
| AFCC KH | 3.50% | Liquid | |
| AFCC RAAS | 4% | Lyophilized | AFOD KH 10 |
| AFCC RAAS | 0.0020% | Lyophilized | AFOD KH 10 |
| AFCC RDNA | 0.00001% | Lyophilized | AFOD KH 10 |

### 1.2 Reagents:

TABLE 6.2
List of reagents

| Reagents/Plates | Vendor | Cat.# |
|---|---|---|
| Reverse Transcriptase | | |
| Avidin standard plates | MSD | MSD-L15AA-6 |
| CHAPS | Pierce | Pierce-28300 |
| EGTA | Sigma | Sigma-E3889-10G |
| DTT | Sigma | Sigma-43815-5G |
| d-ATP | Sigma | Sigma-D6500-10M |
| d-GTP | Sigma | D4010-10MG |
| d-CTP-Na2 | Sigma | D4635-10MG |
| Water (DEPC treated) | Invitrogen | Invitrogen-750023 |
| dry biopD500 primer | Shanghai Shenggong | |
| BSA | Sigma | Sigma-A3294 |
| 4 × Read buffer T | MSD | MSD-R92TD-1 |
| Ru-d-UTP | MSD | Lot: DG2005245071 |
| 96-well round bottom | | |
| PCR tubes | AXYGEN | AXYGEN-PCR-020 |
| PCR tube covers | AXYGEN | AXYGEN-PCR-2CP |

### 1.3 Instrument

Sector Imager S6000 (MesoScale Discovery MSD)
Epmotoin (Eppendorf)
Janus (perkinelmer)
Orbital shaker

### 6. Methods

### 2.1 IC50 Measurement

### 2.2.1 Drug Treatment: Human Plasma Derived Protein Dilutions are Made by Using

EpMotion with 2-fold serial dilutions for 10 concentrations, each in duplicate. a) Add 300 μL of enzyme solution per well of the Costar 96 well plates.

b) Add 5 μL of test article or PBS or DMSO.
c) Seal plate and shake for 2 minutes on an orbital shaker
d) Incubate for 30 minutes on an orbital shaker at room temperature. e) Add 15 μL of the Master Mix to initiate the reaction.
f) Seal plate and shake for 5-10 minutes. g) Incubate at 37 degree for 90 minutes.
h) While this is incubating, add 100 μL of 5% BSA in PBS to the wells of the avidin plates. i) Seal the avidin plates and incubate for 1 hour at room temperature.
j) After the 90 minute incubation, add 60 μL of quenching buffer to the reaction wells. k) Seal the plates and incubate for 5 minutes on the plate shaker.
l) Transfer 500 μL of the well contents to MSD blocked plates (the blocking buffer is simply dumped off. No wash is needed).
m) Incubate MSD plates at RT for 60 minutes.
n) Freshly dilute the 4× read buffer T to 1× using distilled water (not DEPC-treated)
o) Wash MSD plates 3 times with 1500 μL of PBS per well per wash. p) Add 1500 μL of 1× read buffer T to the wells.

### q) Read on the Sector Imager Instrument.

### 2.2.2 Sample or Compound Addition

Test samples were diluted in PBS as 3.5×10⁴ μg/ml stocks. Sample dilutions are made by using Epmotion with 2-fold serial dilutions for 10 concentrations plus PBS (see below for final compound concentrations in the HIV-RT enzyme assay). Reference compound were dissolved in DMSO as 10 mM stocks and dilutions are made by using Epmotion with 3-fold serial dilutions for 10 concentrations plus DMSO (see below for final compound concentrations).

TABLE 6.3
Sample or compound concentrations for IC50 measurement

Name

| | Concentration (ug/ml) | | | | | | | | | |
|---|---|---|---|---|---|---|---|---|---|---|
| AFOD KH | 400 | 200 | 100 | 50 | 25 | 12.5 | 6.25 | 3.1 | 1.6 | 0.8 |
| AFCC KH | 400 | 200 | 100 | 50 | 25 | 12.5 | 6.25 | 3.1 | 1.6 | 0.8 |
| AFCC RAAS 1 | 400 | 200 | 100 | 50 | 25 | 12.5 | 6.25 | 3.1 | 1.6 | 0.8 |
| AFCC RAAS 4 | 400 | 200 | 100 | 50 | 25 | 12.5 | 6.25 | 3.1 | 1.6 | 0.8 |
| AFCC RDNA | 400 | 200 | 100 | 50 | 25 | 12.5 | 6.25 | 3.1 | 1.6 | 0.8 |
| | Concentration (nM) | | | | | | | | | |
| Reference | 100 | 33.3 | 11.1 | 3.7 | 1.2 | 0.4 | 0.1 | 0.05 | 0.02 | 0.01 |
| Compound | | | | | | | | | | |

2.2.3 Data analysis:

Percent of HIV-RT inhibition by protein or compound is calculated using the following equation:

% Inh.=[1−(Signal of sample−Signal of control)/(Signal of DMSO or PBS control−Signal of control)]*100.

Dose-response curves are plotted using Prism

### III. Assay Results

3.1 Raw data from the HIV-RT enzyme assay.

3.1.1 HIV-RT enzyme assay Plate Map*:

Plate 1

| | | | | | | | | | |
|---|---|---|---|---|---|---|---|---|---|
| raw A | raw B | raw C | raw D | raw E | raw F | raw G | | | |
| raw | H | | | | | | | | |
| column | column | column | column | column | column | column | column | column | column |
| | | | | column | column | | | | |
| column | column | column | column | column | column | column | column | column | column |
| | | | | column | column | | | | |

Plate 1:

| | | | | | | | |
|---|---|---|---|---|---|---|---|
| raw A | raw B | raw C | raw D | raw E | raw F | raw G | raw H |

TABLE 6.4
IC50 Summary of the the human plasma derived proteins
and the reference compounds.

| Name | IC50 (ug/ml) | |
|---|---|---|
| AFOD KH | >400 | |
| AFCC KH | 9.89 | |
| AFCC RAAS 1 | 49% inhibition at | |
| | 400 ug/ml | |
| AFCC RAAS 4 | >400 | |
| AFCC RDNA | >400 | |
| | IC50 (nM) | |
| Reference | 0.9 | 1.2 |

### 4. Conclusions

The Z factors of the two plate were 0.84 (plate 1), 0.80 (plate 2), which were much better than QC standard of 0.5. Therefore, the assay data met our QC qualification.

The IC50s of positive control in this study were 0.9 nM (plate 1), 1.2 nM (plate 2) and these results are consistent with our previous data.

### HBV Study

### I. Study Objective: To Test Human Plasma Derived Proteins for Anti-HBV Potency and Cytotoxicity in a Stable HBV Cell Line

### II. Study Protocols

### 1. Materials:

### Cell Line: HepG2.2.15

### 1.2 Samples:

RAAS provided the test articles in the form of dry powder or liquid (Table 7.1). Test samples were diluted in PBS as 3.5×104 μg/ml stocks. Sample dilutions are made by Janus with 2-fold serial dilutions for 8 concentrations plus PBS. Lamivudine is diluted with 3-fold for 9 concentrations.

TABLE 7.1
Sample information

| Name | Protein conc. | Formulation | Diluents |
|---|---|---|---|
| AFOD KH | 10% | Liquid | |
| AFCC KH | 3.50% | Liquid | |
| AFCC RAAS 1 | 4% | Lyophilized | AFOD KH 10 mL |
| AFCC RAAS 4 | 0.0020% | Lyophilized | AFOD KH 10 mL |
| AFCC RDNA | 0.00001% | Lyophilized | AFOD KH 10 mL |

### 1.3 EC50 and CC50 Measurement Test Human Plasma Derived Proteins in the Stable HBV Cell Line HepG2.2.15 for Anti-HBV Potency.

i) Cell culture medium: RPM 1640-4% FBS-1% Pen/Strep-1% Glutamine
ii) HepG2.2.15 cell culture: Grow the cells in T75 flask. Incubated at 37° C., 95% humidity, 5% CO2. Perform 1:3 split every 2-3 days. iii) EC50 measurement:
1) Drug treatment
a) Human plasma derived protein dilutions are made by using Janus with 2-fold serial dilutions for 9 concentrations, each in duplicate.
b) Check cells under microscope.
c) Prepare cell suspension and count cell number. d) Seed the HepG2.2.15 cells into 96-well plates.
e) Treat the cells with cell culture medium containing individual human plasma derived protein 24 hours after cell seeding, the final concentrations of the samples are b shown in Table 7.2.

TABLE 7.2
Name

| | Concentration (ug/ml) | | | | | | | | | |
|---|---|---|---|---|---|---|---|---|---|---|
| AFOD KH | 400 | 200 | 100 | 50 | 25 | 12.5 | 6.25 | 3.1 | 1.6 | 0.8 |
| AFCC KH | 400 | 200 | 100 | 50 | 25 | 12.5 | 6.25 | 3.1 | 1.6 | 0.8 |
| AFCC RAAS | 400 | 200 | 100 | 50 | 25 | 12.5 | 6.25 | 3.1 | 1.6 | 0.8 |
| AFCC RAAS | 400 | 200 | 100 | 50 | 25 | 12.5 | 6.25 | 3.1 | 1.6 | 0.8 |
| AFCC RDNA | 400 | 200 | 100 | 50 | 25 | 12.5 | 6.25 | 3.1 | 1.6 | 0.8 |
| | Concentration (uM) | | | | | | | | | |
| Lamivudine | 2 | 0.6667 | 0.2222 | 0.0741 | 0.0247 | 0.0082 | 0.0027 | 0.000 | 0.000 | 0.0001 |

f) Refresh protein-containing medium on day 3 of drug treatment. g) Collect culture media from the HepG2.2.15 plates on day 6 followed by HBV DNA extraction using QIAamp 96 DNA Blood Kit (QIAGEN #51161).
2) Real time PCR for HBV DNA quantification. a) Dilute HBV plasmid standard by 10-fold from 0.1 ng/ul to 0.000001 ng/ul. b) Prepare realtime PCR mix as shown blow.

| | | Volume for 100 |
|---|---|---|
| PCR reagents | Volume | Reactions |
| DEPC Water | 1.1 ul | 110 ul |
| Taqman Universal Master | 12.5 ul | 1250 ul |
| Mix(2X) | | |
| HBV Primer Forward(50 uM) | 0.2 ul | 20 ul |
| HBV Primer Reverse(50 uM) | 0.2 ul | 20 ul |
| HBV Probe(5 uM) | 1 ul | 100 ul |
| Total | 15 ul | 150 ul |

c) Add 15 ul/well PCR mix to 96-well optical reaction plates.
d) Add 10 ul of the diluted plasmid standard to C12-H12. The amount of HBV DNA in each standard well is: 1 ng, 0.1 ng, 0.01 ng, 0.001 ng, 0.0001 ng, and 0.00001 ng, respectively.
e) Transfer 10 ul of the extracted DNA to the other wells (from Row A-H to the corresponding wells in the optical reaction plates). f) Seal the plates with optical adhesive film.
g) Mix and centrifuge. h) Place the plates into realtime PCR system and set up the program according to the table below.

| | | |
|---|---|---|
| 50° C. | 2 min | 1 cycle |
| 95° C. | 10 min | 1 cycle |
| 95° C. | 15 s | 40 cycle |
| 60° C. | 60 s | |

3) Data analysis: A standard curve is generated by plotting Ct value vs. the amount of the HBV plasmid standard, and the quantity of each sample is estimated based on the Ct value projection on the standard curve; percent of HBV inhibition by protein or compound is calculated using the following equation: % Inh.=[1−(HBV quantity of sample−HBV quantity of HepG2 control)/(HBV quantity of 0% Inhibition control−HBV quantity of HepG2 control)]*100.

### Test Human Plasma Derived Proteins in the Stable HBV Cell Line HepG2.2.15 for Cytotoxicity

i) Cell culture medium: RPM 1640-4% FBS-1% Pen/Strep-1% Glutamine
ii) HepG2.2.15 cell culture: Grow the cells in T75 flask. Incubated at 37° C., 95% humidity, 5% CO2. Perform 1:3 split every 2-3 days. iii) CC50 measurement:
a) Human plasma derived protein dilutions are made by using Janus with 2-fold serial dilutions for 9 concentrations, each in duplicate. b) Check cells under microscope.
c) Prepare cell suspension and count cell number. d) Seed the HepG2.2.15 cells into 96-well plates.
a) Treat the cells with cell culture medium containing individual human plasma derived protein 24 hours after cell seeding, the final concentrations of the samples are shown in Table 2.
e)
f) Refresh protein-containing medium on day 3 of drug treatment.
g) Test cell cytotoxicity on day 6 using CellTiter-Blue Cell Viability Assay KIT.

### III. Assay Results:

TABLE 7.3
EC50 raw data (Plate 1, DNA quantity, ng)

| 1 | | 2 | 3 | 4 | 5 | 6 | 7 | 8 | 9 | 10 | 11 | 12 |
|---|---|---|---|---|---|---|---|---|---|---|---|---|
| Sample final | | | | | | | | | | | | |
| dose (ug/ml) | A 400 | 200 | 100 | 50 | 25 | 12.50 | 6.25 | 3.13 | 1.56 | [symbol] | | 100 |
| | | | | | | | | | | | % inhibition | control |
| AFOD KH | B 0.007 | 0.006 | 0.007 | 0.007 | 0.007 | 0.007 | 0.009 | 0.009 | 0.007 | 0.0100 | | |
| AFOD KH | C 0.006 | 0.005 | 0.005 | 0.006 | 0.007 | 0.006 | 0.006 | 0.007 | 0.007 | 0.0080 | | |
| AFCC KH | D 0.006 | 0.008 | 0.007 | 0.007 | 0.007 | 0.006 | 0.006 | 0.008 | 0.007 | 0.007 | | |
| AFCC KH | E 0.009 | 0.009 | 0.007 | 0.007 | 0.006 | 0.006 | 0.006 | 0.006 | 0.005 | 0.006 | | |
| AFCC RAAS 1 | F 0.006 | 0.005 | 0.005 | 0.005 | 0.006 | 0.005 | 0.007 | 0.006 | 0.007 | 0.007 | | |
| AFCC RAAS 1 | G 0.008 | 0.006 | 0.002 | 0.006 | 0.009 | 0.008 | 0.008 | 0.008 | 0.008 | 0.009 | | |
| | H | | | | | | | | | | | |

[symbol]

indicates data missing or illegible when filed

TABLE 7.4
EC50 raw data(Plate 2, DNA quantity, ng)

| 1 | | 2 | 3 | 4 | 5 | 6 | 7 | 8 | 9 | 10 | 11 | 12 |
|---|---|---|---|---|---|---|---|---|---|---|---|---|
| Sample final | | | | | | | | | | | | |
| dose (ug/ml) | A 400 | 200 | 100 | 50 | 25 | 12.50 | 6.25 | 3.13 | 1.56 | [symbol] | | 100 |
| | | | | | | | | | | | % inhibition | control |
| AFCC RAAS 4 | B 0.008 | 0.007 | 0.008 | 0.008 | 0.007 | 0.007 | 0.009 | 0.009 | 0.009 | 0.012 | | 0 |
| AFCC RAAS 4 | C 0.007 | 0.006 | 0.006 | 0.007 | 0.007 | 0.007 | 0.007 | 0.008 | 0.007 | 0.008 | | 0 |
| AFCC RDNA | D 0.007 | 0.006 | 0.007 | 0.007 | 0.006 | 0.006 | 0.007 | 0.007 | 0.008 | 0.008 | | |
| AFCC RDNA | E 0.007 | 0.007 | 0.006 | 0.007 | 0.007 | 0.006 | 0.007 | 0.006 | 0.007 | 0.007 | | |
| Lamivudine | F 0.001 | 0.001 | 0.001 | 0.002 | 0.003 | 0.005 | 0.007 | 0.011 | 0.010 | 0.009 | | |
| Lamivudine | G 0.001 | 0.001 | 0.001 | 0.002 | 0.004 | 0.007 | 0.010 | 0.012 | 0.014 | 0.011 | | |

| Lamivudine | | | | | | | | | |
|---|---|---|---|---|---|---|---|---|---|
| final dose (uM) | H 2 | 0.6667 | 0.2222 | 0.0741 | 0.0247 | 0.0082 | 0.0027 | 0.0009 | 0.0003 |

[symbol]

indicates data missing or illegible when filed

TABLE 7.5
CC50 raw data (Plate 1)

| | | 1 | 2 | 3 | 4 | 5 | 6 | 7 | 8 | 9 | 10 | 11 | 12 |
|---|---|---|---|---|---|---|---|---|---|---|---|---|---|
| Sample | A | 400 | 200 | 100 | 50 | 25 | 12.50 | 6.25 | 3.13 | 1.56 | 0 | DMEM | |
| final | | | | | | | | | | | | | |
| dose | | | | | | | | | | | | | |
| (ug/ml) | | | | | | | | | | | | | |
| AFOD | B | 55803 | 64797 | 71230 | 72149 | 77139 | 78592 | 78704 | 79161 | 79842 | 81561 | 1188 | |
| KH | | | | | | | | | | | | | |
| AFOD | C | 56823 | 68233 | 70631 | 71131 | 76688 | 73011 | 77956 | 78420 | 76152 | 81682 | 1183 | |
| KH | | | | | | | | | | | | | |
| AFCC | D | 82228 | 84496 | 82896 | 80731 | 79344 | 81008 | 80922 | 80895 | 77356 | 79034 | 1193 | |
| KH | | | | | | | | | | | | | |
| AFCC | E | 81506 | 77561 | 74728 | 80403 | 73910 | 82101 | 83557 | 76077 | 74991 | 82662 | 1168 | |
| KH | | | | | | | | | | | | | |
| AFCC | F | 66408 | 74144 | 78364 | 78223 | 76486 | 77972 | 75031 | 78457 | 66609 | 70886 | 1161 | |
| RAAS 1 | | | | | | | | | | | | | |
| AFCC | G | 67246 | 72750 | 74032 | 78193 | 78179 | 76672 | 80360 | 79757 | 69473 | 77564 | 1170 | |
| RAAS 1 | | | | | | | | | | | | | |
| | H | | | | | | | | | | | | |

Note:
DMEM-100% inhibition control

TABLE 7.6
CC50 raw data (Plate 1)

| | | 1 | 2 | 3 | 4 | 5 | 6 | 7 | 8 | 9 | 10 | 11 | 12 |
|---|---|---|---|---|---|---|---|---|---|---|---|---|---|
| Sample | A | 400 | 200 | 100 | 50 | 25 | 12.50 | 6.25 | 3.13 | 1.56 | 0 | DMEM | |
| final | | | | | | | | | | | | | |
| dose | | | | | | | | | | | | | |
| (ug/ml) | | | | | | | | | | | | | |
| AFCC | B | 59995 | 63891 | 66637 | 71746 | 75856 | 77487 | 77999 | 77411 | 78544 | 80078 | 1180 | |
| RAAS 4 | | | | | | | | | | | | | |
| AFCC | C | 59231 | 62440 | 63030 | 65439 | 66946 | 71795 | 73718 | 71840 | 77588 | 80043 | 1164 | |
| RAAS 4 | | | | | | | | | | | | | |
| AFCC | D | 56862 | 67068 | 68782 | 69716 | 65137 | 76510 | 76774 | 76077 | 74595 | 76353 | 1171 | |
| RDNA | | | | | | | | | | | | | |
| AFCC | E | 57147 | 67735 | 68921 | 70400 | 70840 | 73754 | 72536 | 70262 | 74724 | 79979 | 1145 | |
| RDNA | | | | | | | | | | | | | |
| Lamivudine | F | 76444 | 68943 | 69121 | 70533 | 71070 | 72010 | 72631 | 71985 | 75415 | 77856 | 1154 | |
| Lamivudine | G | 80622 | 80415 | 78596 | 77036 | 77288 | 78038 | 78269 | 78178 | 78055 | 80851 | 1152 | |
| | H | | | | | | | | | | | | |

Note:
DMEM-100% inhibition control

### IV. Conclusions

The EC50 of the positive control Lamivudine in this study is 0.0062 uM, which is consistent with our previous data.

### In Vivo Study:

Efficacy of FibrinGluRAAS plus AFOD Study was performed at one of the top ten CRO.

### RAAS

### Title:

Anti-tumor efficacy of high concentrated fibrinogen enriched a1at thrombin and Afod in a patient-derived tumor xenograft (PDX) model of lung cancer in nude mice.

### Description:

Patient-derived tumor xenograft (PDX) model of lung cancer was used to evaluate the anti-cancer efficacy of high concentrated fibrinogen enriched a1at thrombin and Afod at different 3 doses. The results showed that high concentrated fibrinogen enriched a1at thrombin and afod at all doses significantly inhibited the growth of PDX tumors implanted at 4 different locations of the peritoneum while having minor effects on mice body weights, which indicates high concentrated fibrinogen enriched a1at thrombin and Afod is a potent anti-cancer agent on lung cancer with a limited side effect.

### Subject:

high concentrated fibrinogen enriched a1at thrombin and Afod, patient-derived tumor xenograft model, lung cancer

### Quotation:

### RAAS-20111029

### SUMMARY

Patient-derived tumor xenograft (PDX) model of lung cancer (LU-01-0032) was used to evaluate the anti-tumor efficacy of high concentrated fibrinogen enriched a1at thrombin and Afod at 3 doses. PDX tumors (LU-01-0032) were implanted at 4 different locations in peritoneal cavity, and high concentrated fibrinogen enriched a1at thrombin and Afod or a control agent was applied to peritoneum before and after tumor implantation. Forty five days after implantation, the mice were sacrificed and tumors were removed and weighed. The final tumor weights for all groups were statistically analyzed by one-way ANOVA with the significance level set at 0.05.

The data show that high concentrated fibrinogen enriched a1at thrombin and Afod at all 3 doses exhibits significant inhibitory effects on tumor growth in the lung cancer model while no significant toxicity was observed, which indicates high concentrated fibrinogen enriched a1at thrombin and Afod was a potential anti-tumor agent in lung cancer, warranting further development of high concentrated fibrinogen enriched a1at thrombin and Afod for clinical application.

Note: The page numbers presented in this table of contents are not consistent with the page numbering in this specification.

TABLE OF CONTENTS

| 1. | DETAILS OF FACILITY, PERSONNEL AND DATA LOCATION | 4 |
|---|---|---|
| 2. | INTRODUCTION | 4 |
| 3. | METHODS | 5 |
| 3.1. | Experimental Preparations | 5 |
| 3.1.1. | Animal preparation | 5 |
| 3.1.2. | Tumor tissue preparation | 5 |
| 3.1.3. | Formulation | 5 |
| 3.2. | Experimental Protocol | 5 |
| 3.2.1. | Establishment of Xenograft Model and Treatment | 5 |
| 3.2.2. | Evaluation of the Anti-Tumor Activity | 7 |
| 3.3. | Drugs and Materials | 8 |
| 3.4. | Data Analysis | 8 |
| 3.4.1. | Relative Chage of Body Weight (RCBW) | 8 |
| 3.4.2. | Tumor weight | 8 |
| 3.4.3. | Statistical analysis | 8 |
| 4. | RESULTS | 8 |
| 4.1. | Tumor growth inhibition | 8 |
| 4.2. | Effect on Body weight | 9 |
| 5. | DISCUSSION | 9 |
| 6. | REFERENCES | 10 |
| 7. | FIGURES | 11 |
| FIG. 26.18. Anti-tumor efficacy of high concentrated fibrinogen enriched a1at thrombin and | | 11 |
| Afod in PDX model LU-01-0032. | | |
| FIG. 26.22. Photographs of tumors dissected from abdominal cavity of each group. | | 12 |
| FIG. 26.23. Ratios of mice with palpable tumors observed in each group. | | 13 |
| FIG. 26.24. Relative change of body weight (%) of different groups. | | 14 |
| 8. | TABLES | 15 |

| | |
|---|---|
| Table 8.2. Ratios of palpable tumors observed in each group. | 15 |
| Table 8.3. Relative change of body weight (%) of different groups. | 16 |

### 1. Details of Facility, Personnel and Data Location

| | |
|---|---|
| Sponsor: | RAAS |
| Test Facility: | WuXi AppTec Animal facility in |
| | 90 Delin Road, |
| | Waigaoqiao Free Trade Zone, |
| | Shanghai 200131, P.R.China. |
| Date of Work: | Commenced: Oct. 17, 2011 |
| | Completed: Nov. 25, 2011 |
| Personnel Involved: | Yunbiao Yan scientist BS |
| | Guizhu Yang scientist BS |
| Study Director/Senior Scientist: | Douglas Fang Senior director Ph.D |

### Location of Raw Data, Original Protocols, Experimental Details and Report

The studies described in this report were carried out on behalf of RAAS at external laboratories:

All raw data, protocols and experimental details pertaining to these studies and the original of the report will be held in the Archive of WuXi AppTec in 90 Delin Road, Waigaoqiao Free Trade Zone, Shanghai 200131, P.R. China.

### 2. Introduction

The aim of the study was to test anti-tumor efficacy of high concentrated fibrinogen enriched a1at thrombin and Afod in patient-derived lung tumor xenograft (PDX) model in nude mice.

The model used in the study was derived from surgically resected, fresh patient tumor tissues. The first generation of the xenograft tumors in mice was termed passage 0 (P0), and so on during continual implantation in mice. The passage of xenograft tumors at P5 (LU-01-0032) were used in this study.

All the experiments were conducted in the AAALAC-accrediated animal facility in compliance with the protocol approved by the Institutional Animal Care and Use Committee (IACUC).

### 3. Methods

### 3.1. Experimental Preparations

### 3.1.1. Animal Preparation

Female Balb/c nude mice, with a body weight of approximately 20 grams, were obtained from an approved vendor (Sino-British SIPPR/BK Lab. Animal Co. Ltd., Shanghai, China).

### Acclimation/Quarantine:

Upon arrival, animals were assessed as to their general health by a member of a veterinary staff or authorized personnel. Animals were acclimated for at least 3 days (upon arrival at the experiment room) before being used for the study.

### Animal Husbandry:

Animals were housed in groups during acclimation and individually housed during in-life. The animal room environment was adjusted to the following target conditions: temperature 20 to 25° C., relative humidity 40 to 70%, 12 hours artificial light and 12 hours dark. Temperature and relative humidity was monitored daily.

All animals had access to Certified Rodent Diet (Sino-British SIPPR/BK Lab. Animal Co. Ltd., Shanghai, China) ad libitum. Animals were not fasted prior to the study. Water was autoclaved before provided to the animals ad libitum. Periodic analyses of the water were performed and the results were archived at WuXi AppTec. There were no known contaminants in the diet or water which, at the levels detected expected to interfere with the purpose, conduct or outcome of the study.

### 3.1.2. Tumor Tissue Preparation

The lung xenograft tumor models were established from surgically resected clinical tumor samples. The first generation of the xenograft tumors in mice is termed passage 0 (P0), and so on during continual implantation in mice. The tumor tissues at passage 5 (LU-01-0032) were used in this study.

### 3.1.3. Formulation

High concentrated fibrinogen enriched a1at thrombin and Afod were provide by RAAS and prepared by RAAS scientist during experiment before use. Matrigel (BD Biosciences; cat. #356234).

### 3.2. Experimental Protocol

### 3.2.1. Establishment of Xenograft Model and Treatment

### Grouping and Treatment

Nude mice were assigned to 6 different groups with 11-19 mice/group and each group received different treatments as shown in Table 8.1.

TABLE 8.1
Grouping and the treatment.

| Group | Treatment | N | Remarks |
|---|---|---|---|
| 1 | Sham-operation | 12 | Open up the abdominal cavity and close |
| | | | it with sutures. (No implants) |
| 2 | Vehicle control | 13 | Implant tumor fragments of 20 mm³ in |
| | | | size into 4 corners of abdominal cavity. |
| | | | Close body with sutures. |
| 3 | Matrigel | 13 | Embed tumor fragments of 20 mm³ in |
| | | | Matrigel. Implant the tumor fragments into 4 |
| | | | corners of abdominal cavity. Close body with |
| | | | sutures. |
| 4 | 3 ml high | 19 | Spray high concentrated fibrinogen |
| | concentrated | | enriched |
| | enriched a1at thrombin and | | a1at thrombin and Afod to cover the entire |
| | Afod | | peritoneum and the internal organs. Implant the |
| | (high dose) on the | | tumor fragments of |
| | peritoneum in abdominal | | 20 mm³ into 4 |
| | cavity of | | corners of abdominal cavity. Close body |
| | nude mice | | with sutures. |
| 5 | 2 ml high | 14 | Spray high concentrated fibrinogen |
| | concentrated | | enriched |
| | fibrinogen enriched | | a1at thrombin and Afod to cover the entire |
| | a1at thrombin | | peritoneum and the internal organs. Implant the |
| | and Afod (moderate | | tumor fragments of |
| | dose) on the | | 20 mm³ into 4 |
| | peritoneum in abdominal | | corners of abdominal cavity. Close body with |
| | cavity of nude mice | | sutures. |
| 6 | 1 ml high | 11 | Spray high concentrated fibrinogen |
| | concentrated | | enriched |
| | fibrinogen enriched | | a1at thrombin and Afod to cover the entire |
| | a1at thrombin | | peritoneum and the internal organs. Implant the |
| | and Afod (low dose) on | | tumor fragments of |
| | the peritoneum in abdominal | | 20 mm³ into 4 |
| | cavity of nude mice | | corners of abdominal cavity. Close body with |
| | | | sutures. |
| Total | | 82 | |

### Experiment Procedures

A. Measured the body weight of each mouse before surgery.
B. The animal was anesthetized by i.p. injection of sodium pentobarbital at 60-70 mg/kg. Disinfect the abdominal skin of nude mice with 70% ethanol solution. Open up the abdominal wall along the midline of the ventral surface to expose the peritoneal surface.
C. The surgeries for different groups were done according to table 8.1.
D. For groups using test agent high concentrated fibrinogen enriched a1at thrombin and Afod, the test agent was then applied on the peritoneal surface.
E. Tumor fragments were implanted at 4 different locations of the peritoneal cavity.

The test agent acted as a glue to hold the fragments.

F. The test agent high concentrated fibrinogen enriched a1at thrombin and Afod was applied again on the surface of tumor fragments and peritoneum.
G. After the fibrin membrane formed completely, the peritoneal cavity was closed.
H. In Matrigel control groups, tumor fragments were embedded into matrigel before implantation.
I. Postoperative cares followed protocol SOP-BEO-0016-1.0.
J. Mice were palpated for tumors 2 weeks after implantation. The ratio of palpable tumors observed in each group was recorded.
K. Forty five days after implantation, the mice were sacrificed and tumors were dissected and weighed.
L. The tissues surrounding tumor fragments were also checked to find out whether the tumors had spread to other organ sites within the peritoneal cavity.
M. Pictures of tumor-bearing mice and dissected tumors were taken.
N. If possible, tumor sizes were measured twice per week. Tumor volumes (mm³) are obtained by using the following formula: volume=(W2×L)/2 (W, width; L, length in mm of the tumor).
O. During the experiment, health conditions of mice were observed daily. Body weights of mice were monitored twice per week.

### 3.2.2. Evaluation of the Anti-Tumor Activity

Health conditions of mice were observed daily. Body weights were measured twice per week during the treatment. Mice were palpated for tumors 2 weeks after implantation. The ratio of palpable tumors observed in each group was recorded.

45 days after treatment, all mice were euthanized with CO₂ and cervical dislocation was followed after respiratory arrest. Routine necropsy was performed to detect any abnormal signs of each internal organ with specific attention to metastases. Each tumor was removed and weighted.

### 3.3. Drugs and Materials

High concentrated fibrinogen enriched a1at thrombin and Afod were provided by RAAS; Matrigel was from BD Biosciences (San Jose, Calif., cat. #356234). Digital caliper was from Sylvac, Switzerland.

### 3.4. Data Analysis

### 3.4.1. Relative Change of Body Weight (RCBW)

Relative change of body weight (RCBW) was calculated based on the following formula: RCBW (%)=(BWi−BW0)/BW0×100%; BWi was the body weight on the day of weighing and BW0 was the body weight before surgery.

3.4.2. Tumor weight

Tumors from each mouse were pooled and weighed after sacrificing mice.

3.4.3. Statistical analysis

Data were expressed as mean±SEM; the difference between the groups was analyzed for significance using one-way ANOVA and Dunnett's test.

### 4. Results

4.1. Tumor growth inhibition

Four weeks after implantation, 9 out of 13 mice in vehicle control group showed palpable tumors, while only less than 5 palpable tumors were found in each high concentrated fibrinogen enriched a1at thrombin and Afod-treated group. High concentrated fibrinogen enriched a1at thrombin and Afod treatment delayed the appearance of palpable tumors as shown in table 8.2, indicating high concentrated fibrinogen enriched a1at thrombin and Afod inhibited the growth of implanted lung tumors in vivo. After sacrificing the mice, tumors were found in all the mice in vehicle control group, while some tumors completely regressed in several high concentrated fibrinogen enriched a1at thrombin and Afod-treated mice (FIG. 26.23).

Forty-five days after implantation, tumors in vehicle control group reached more than 0.7 g on average. Conversely, tumor weights in high concentrated fibrinogen enriched a1at thrombin and Afod high, moderate and low dose groups were 0.19 g, 0.16 g and 0.16 g, respectively. Compared with the vehicle control, high concentrated fibrinogen enriched a1at thrombin and Afod demonstrated significant anti-tumor activities in lung cancer PDX model at all 3 doses (FIGS. 26.18-26.19).

The inhibition on tumor growth were shown in FIGS. 26.18-26.20 and table 8.2.

### 4.2. Effect on Body Weight

Loss of body weight, a sign of toxicity, was not seen in high concentrated fibrinogen enriched a1at thrombin and Afod-treated groups, indicating the test agent has no/little side effects.

The effect on body weight was shown in FIG. 26.24 and table 8.3.

### 5. Discussion

Patient-derived tumor xenograft (PDX) model of lung cancer was used to evaluate the anti-cancer efficacy of the high concentrated fibrinogen enriched a1at thrombin and Afod at 3 doses. PDX tumors (LU-01-0032) were implanted at 4 different locations in peritoneal cavity, and high concentrated fibrinogen enriched a1at thrombin and Afod or a control agent was applied to peritoneum before and after tumor implantation.

Mice were palpated for tumors 2 weeks after implantation. The ratio of palpable tumors observed in each group was recorded. High concentrated fibrinogen enriched a1at thrombin and Afod treatment inhibited the tumor growth as shown by the delayed appearance of palpable tumors and decreased tumor incidence. Four weeks after implantation, 9 out of 13 mice in vehicle control group showed palpable tumors, while only less than 5 palpable tumors were found in each high concentrated fibrinogen enriched a1at thrombin and Afod-treated group (Table 8.2).

Forty-five days after implantation, the mice were sacrificed and tumors were dissected and weighed. After sacrificing the mice, tumors were found in all the mice in vehicle control group, while some tumors completely regressed in several high concentrated fibrinogen enriched a1at thrombin and Afod-treated mice. Tumors in vehicle control group reached more than 0.7 g on average. Conversely, tumor weights in high concentrated fibrinogen enriched a1at thrombin and Afod high, moderate and low dose groups were 0.19 g, 0.16 g and 0.16 g, respectively. Compared with the vehicle control, high concentrated fibrinogen enriched a1at thrombin and Afod demonstrated significant anti-tumor activities in lung cancer PDX model at all 3 doses. Matrigel has been commonly used to facilitate the establishment of human tumor xenografts in rodents. In this study, matrigel group also showed a significant inhibitory effect on tumor weight.

Loss of body weight, a sign of toxicity, was not seen in all high concentrated fibrinogen enriched a1at thrombin and Afod-treated groups, indicating the test agent has no/little side effects.

In summary, the results show that high concentrated fibrinogen enriched a1at thrombin and Afod at all doses significantly inhibits the growth of lung tumors in vivo while having minor effects on mice body weight. The results suggest that high concentrated fibrinogen enriched a1at thrombin and Afod is a potent anti-tumor agent in lung cancer.

### 6. References

N/A

### 7. Figures

FIG. 26.18. Anti-Tumor Efficacy of High Concentrated Fibrinogen Enriched a1at Thrombin And Afod in PDX Model LU-01-0032.
0.0

Tumor weights from model LU-01-0032 were used. Data are expressed as mean±SEM. *<0.05, **<0.01, ***<0.001 vs vehicle group (one-way ANOVA and Dunnett's test).

### Confidential

FIG. 26.22. Photographs of Tumors Dissected from Abdominal Cavity of Each Group.

Tumors from each mouse of model LU-01-0032 were pooled and weighed. Scale bar, 1 cm. A, sham-operated; control; C, matrigel; D, test agent high dose; E, test agent moderate dose; F, test agent low dose.

### Confidential

FIG. 26.23. Ratios of Mice with Palpable Tumors Observed in Each Group.

After sacrificing the mice, the tumors from each mouse of model LU-01-0032 were pooled and the ratios of mice bearing tumors in each group were recorded.

### 13

### Confidential

### FIG. 26.24. Relative Change of Body Weight (%) of Different Groups.

Data are expressed as mean±SEM. Relative change of body weight (RCBW) was calculated based on the following formula: RCBW (%)=(BWi−BW0)/BW0×100%; BWi was the body weight on the day of weighing and BW0 was the body weight before surgery.

### Confidential

### 8. Tables

TABLE 8.2
Ratios of palpable tumors observed in each group.

| | | | | | | | | | | | | | Actual |
|---|---|---|---|---|---|---|---|---|---|---|---|---|---|
| | | Palpable tumor observed (days after surgery) | | | | | | | | | | | incidence |
| Gr ou | | 15 | 19 | 22 | 24 | 26 | 29 | 33 | 36 | 40 | 43 | 45 | at the end |
| 1 | Sham- operated | 0/12 | 0/12 | 0/12 | 0/12 | 0/12 | 0/12 | 0/12 | 0/12 | 0/12 | 0/12 | 0/12 | 0/12 |
| 2 | vehicle control | 3/13 | 6/13 | 7/13 | 8/13 | 8/13 | 9/13 | 9/13 | 9/13 | 9/13 | 10/1 | 10/1 | 13/13 |
| 3 | positive control | 1/13 | 4/13 | 6/13 | 6/13 | 6/13 | 6/13 | 6/13 | 6/13 | 6/13 | 6/13 | 6/13 | 12/13 |
| 4 | high dose of | 0/19 | 0/19 | 0/19 | 0/19 | 1/19 | 3/19 | 3/19 | 4/19 | 5/19 | 8/19 | 8/19 | 15/19 |
| | test agent | | | | | | | | | | | | |
| 5 | moderate dose | 0/14 | 0/14 | 1/14 | 1/14 | 2/14 | 3/14 | 3/14 | 3/14 | 6/14 | 6/14 | 7/14 | 10/14 |
| | of test agent | | | | | | | | | | | | |
| 6 | low dose of | 1/11 | 2/11 | 4/11 | 4/11 | 5/11 | 5/11 | 5/11 | 5/11 | 5/11 | 5/11 | 5/11 | 10/11 |
| | test agent | | | | | | | | | | | | |

Mice were palpated for tumors at 15, 19, 22, 24, 26, 29, 33, 36, 40, 43, and 45 days after implantation. The ratios of palpable tumors observed in each group were recorded.

TABLE 8.3
Relative change of body weight (%) of different groups.

| | | Days after | | | | | | | | | |
|---|---|---|---|---|---|---|---|---|---|---|---|
| | | 0 | 1 | 2 | 3 | 4 | 5 | 6 | 7 | 8 | 15 |
| | | Group | | | | | | | | | |
| | | RC | RC | RC | RC | RC | RC | RC | RC | RC | RC |
| | | B W | B W | B W | B W | B W | B W | B W | B W | B W | B W |
| Sham- | Mean | — | [symbol] | — | [symbol] | [symbol] | [symbol] | [symbol] | [symbol] | [symbol] | 11.4 |
| operated | SD | 2.0 | 2.9 | 3.7 | 3.2 | 4.4 | 4.4 | 5.1 | 4.2 | 5.1 | 4.3 |
| group | SEM | 0.60 | 0.85 | 1.08 | 0.93 | 1.29 | 1.27 | 1.48 | 1.24 | 1.47 | 1.24 |
| Vehicle | Mean | — | — | — | — | 1.8 | 2.9 | 5.4 | 6.7 | 7.7 | 11.1 |
| control | SD | 0.71 | 3.19 | 2.83 | 2.41 | 3.03 | 3.03 | 3.78 | 4.18 | 4.57 | 5.56 |
| group | SEM | 0.20 | 0.88 | 0.78 | 0.67 | 0.84 | 0.84 | 1.05 | 1.16 | 1.27 | 1.54 |
| | Mean | 0.5 | — | — | — | 1.3 | 2.3 | 5.1 | 5.7 | 6.8 | 10.8 |
| Matrigel | SD | 0.70 | 4.50 | 3.91 | 3.56 | 3.72 | 3.91 | 3.24 | 3.14 | 3.48 | 4.92 |
| group | SEM | 0.19 | 1.25 | 1.08 | 0.99 | 1.03 | 1.08 | 0.90 | 0.87 | 0.96 | 1.37 |
| | Mean | 13.6 | — | — | — | — | 1.3 | 4.2 | 3.9 | 6.1 | 14.2 |
| Test agent | SD | 1.28 | 2.95 | 4.08 | 3.45 | 3.59 | 4.07 | 3.86 | 3.85 | 3.28 | 3.10 |
| high dose | SEM | 0.29 | 0.68 | 0.94 | 0.79 | 0.82 | 0.93 | 0.89 | 0.88 | 0.75 | 0.71 |
| Test agent | Mean | 9.7 | — | — | — | — | 0.4 | 3.2 | 5.9 | 6.2 | 10.5 |
| moderate | SD | 0.87 | 3.06 | 3.70 | 2.82 | 3.32 | 2.82 | 3.03 | 4.07 | 2.25 | 2.65 |
| dose | SEM | 0.23 | 0.82 | 0.99 | 0.75 | 0.89 | 0.75 | 0.81 | 1.09 | 0.60 | 0.71 |
| | Mean | 2.9 | — | — | — | — | 1.7 | 4.1 | 5.2 | 5.6 | 14.5 |
| Test agent | SD | 2.88 | 2.48 | 2.73 | 3.47 | 3.97 | 3.40 | 4.03 | 3.53 | 3.69 | 4.36 |
| low dose | SEM | 0.80 | 0.69 | 0.76 | 0.96 | 1.10 | 1.03 | 1.22 | 1.06 | 1.11 | 1.31 |

| | | Days after | | | | | | | | |
|---|---|---|---|---|---|---|---|---|---|---|
| | | 19 | 22 | 26 | 29 | 33 | 36 | 40 | 43 | 45 |
| | | Group | | | | | | | | |
| | | RC | RC | RC | RC | RC | RC | RC | RC | RC |
| | | B W | B W | B W | B W | B W | B W | B W | B W | B W |
| Sham- | Mean | [symbol] | 15.3 | [symbol] | [symbol] | [symbol] | [symbol] | [symbol] | 23.0 | [symbol] |
| operated | SD | 4.1 | 4.0 | 4.5 | 4.3 | 4.4 | 3.60 | 3.4 | 3.67 | 4.32 |
| group | SEM | 1.19 | 1.17 | 1.32 | 1.25 | 1.27 | 1.04 | 0.99 | 1.06 | 1.25 |
| Vehicle | Mean | 14.4 | 14.7 | 16.2 | 17.3 | 19.7 | 18.3 | 22.5 | 23.2 | 22.3 |
| control | SD | 4.47 | 4.45 | 3.63 | 4.92 | 5.70 | 5.49 | 6.93 | 7.50 | 6.86 |
| group | SEM | 1.24 | 1.23 | 1.01 | 1.36 | 1.58 | 1.52 | 1.92 | 2.08 | 1.90 |
| | Mean | 15.1 | 17.4 | 17.9 | 18.7 | 21.4 | 20.1 | 23.7 | 25.3 | 23.3 |
| Matrigel | SD | 5.03 | 5.55 | 4.66 | 5.92 | 6.37 | 6.68 | 5.84 | 5.28 | 5.64 |
| group | SEM | 1.40 | 1.54 | 1.29 | 1.64 | 1.77 | 1.85 | 1.62 | 1.47 | 1.56 |
| | Mean | 16.0 | 16.6 | 18.0 | 19.0 | 21.1 | 19.2 | 23.3 | 24.6 | 23.2 |
| Test agent | SD | 2.77 | 3.39 | 3.42 | 3.31 | 3.63 | 4.03 | 4.08 | 4.66 | 4.64 |
| high dose | SEM | 0.64 | 0.78 | 0.78 | 0.76 | 0.83 | 0.92 | 0.94 | 1.07 | 1.06 |
| Test agent | Mean | 12.5 | 13.6 | 15.5 | 17.8 | 19.3 | 17.8 | 20.4 | 22.6 | 21.9 |
| moderate | SD | 2.90 | 3.46 | 3.87 | 4.27 | 4.31 | 4.01 | 2.98 | 3.72 | 4.80 |
| dose | SEM | 0.78 | 0.93 | 1.03 | 1.14 | 1.15 | 1.07 | 0.80 | 1.00 | 1.28 |
| | Mean | 16.9 | 18.5 | 20.1 | 21.6 | 24.4 | 21.9 | 25.4 | 27.3 | 26.4 |
| Test agent | SD | 3.75 | 4.06 | 4.34 | 5.72 | 6.59 | 5.54 | 5.93 | 6.01 | 7.15 |
| low dose | SEM | 1.13 | 1.22 | 1.31 | 1.73 | 1.99 | 1.67 | 1.79 | 1.81 | 2.15 |

[symbol]

indicates data missing or illegible when filed

Relative change of body weight (RCBW) was calculated based on the following formula:

RCBW(%)=(BWi−BW0)/BW0×100%;

BWi was the body weight on the day of weighing and BW0 was the body weight before surgery.

### RAAS

### Title:

Anti-tumor efficacy of high concentrated fibrinogen enriched a1at thrombin and AFOD in patient-derived tumor xenograft (PDX) models in nude mice.

### Description:

Patient-derived colorectal tumor xenograft (PDX) model was used to evaluate the anti-cancer efficacy of the high concentrated fibrinogen enriched a1at thrombin and AFOD at different 3 doses. The results showed that high concentrated fibrinogen enriched a1at thrombin and AFOD at all doses significantly inhibited the growth of PDX tumors implanted at 4 different locations of the peritoneum while having minor effects on mice body weights, which indicated high concentrated fibrinogen enriched a1at thrombin and AFOD is a potent anti-cancer agent on colorectal cancer with a limited side effect.

### Subject:

high concentrated fibrinogen enriched a1at thrombin and AFOD, fibrinogen, thrombin, patient-derived tumor xenograft model, colorectal cancer

### Quotation:

RAAS-20110926

### Summary

Patient-derived colorectal tumor xenograft (PDX) models (CO-04-0001 or CO-04-0002) were used to evaluate the anti-tumor efficacy of high concentrated fibrinogen enriched a1at thrombin and Afod at 3 doses. PDX tumors (CO-04-0001 or CO-04-0002) were implanted at 4 different locations in peritoneal cavity, and high concentrated fibrinogen enriched a1at thrombin and Afod, or a control agent was applied to peritoneum before and after tumor implantation. 30 days after implantation, the mice were sacrificed and tumors were dissected and weighed. The final tumor weights for all groups were statistically analyzed by one-way ANOVA with the significance level set at 0.05.

The data show that high concentrated fibrinogen enriched a1at thrombin and Afod at all 3 doses exhibits significant inhibitory effects on tumor growth in PDX colorectal cancer model while no significant toxicity was observed, which indicates high concentrated fibrinogen enriched a1at thrombin and Afod is a potential anti-tumor agent in colorectal cancer, warranting further development of the agent for clinical application.

Note: The page numbers presented in this table of contents are not consistent with the page numbering in this specification.

TABLE OF CONTENTS

| 1. | DETAILS OF FACILITY, PERSONNEL AND DATA | 4 |
|---|---|---|
| | LOCATION | |
| 2. | INTRODUCTION | 4 |
| 3. | METHODS | 5 |
| 3.1. | Experimental Preparations | 5 |
| 3.1.1. | Animal preparation | 5 |
| 3.1.2. | Tumor tissue preparation | 5 |
| 3.1.3. | Formulation | 5 |
| 3.2. | Experimental Protocol | 5 |
| 3.2.1. | Establishment of Xenograft Model and Treatment | 5 |
| 3.2.2. | Evaluation of the Anti-Tumor Activity | 8 |
| 3.3. | Drugs and Materials | 8 |
| 3.4. | Data Analysis | 8 |
| 3.4.1. | Relative Chage of Body Weight (RCBW) | 8 |
| 3.4.2. | Tumor weight | 8 |
| 3.4.3. | Statistical analysis | 8 |
| 4. | RESULTS | 8 |
| 4.1. | Inhibition on tumor growth | 8 |
| 4.2. | Effect on Body weight | 9 |
| 5. | DISCUSSION | 9 |
| 6. | REFERENCES | 11 |
| 7. | FIGURES | 12 |
| FIG. 26.18. Anti-tumor efficacy of test agent in PDX model CO-04- | | 12 |
| 0002 | | |
| FIG. 26.22. Anti-tumor efficacy of test agent in PDX model CO-04- | | 13 |
| 0002 and CO-04-0001. | | |
| FIG. 26.23. Photographs of tumors dissected from abdominal cavity of | | 14 |
| each group. | | |
| FIG. 26.24. Relative change of body weight (%) of different groups. | | 15 |
| 8. | TABLES | 16 |

| | |
|---|---|
| Table 8.2. Ratios of palpable tumors observed in each group | 16 |
| Table 8.3. Relative change of body weight (%) of different groups. | 17 |

### 1. Details of Facility, Personnel and Data Location

| | |
|---|---|
| Sponsor: | RAAS |
| Test Facility: | WuXi AppTec Animal facility in |
| | 90 Delin Road, Waigaoqiao Free Trade |
| | Zone, Shanghai 200131, P.R.China. |
| Date of Work: | Commenced: Oct. 17, 2011 |
| | Completed: Nov. 25, 2011 |
| Personnel Involved: | Yunbiao Yan scientist BS |
| | Guizhu Yang scientist BS |
| Study Director/Senior Scientist: | Douglas Fang Senior director Ph.D |

### Location of Raw Data, Original Protocols, Experimental Details and Report

The studies described in this report were carried out on behalf of RAAS at external laboratories:

All raw data, protocols and experimental details pertaining to these studies and the original of the report will be held in the Archive of WuXi AppTec in 90 Delin Road, Waigaoqiao Free Trade Zone, Shanghai 200131, P.R. China.

### 2. Introduction

The aim of the study was to test anti-tumor efficacy of high concentrated fibrinogen enriched a1at thrombin and Afod in patient-derived colorectal tumor xenograft (PDX) model in nude mice.

The model used in the study was derived from surgically resected, fresh patient tumor tissues. The first generation of the xenograft tumors in mice was termed passage 0 (P0), and so on during continual implantation in mice. The passage of xenograft tumors at P2 (CO-04-0002) or P3 (CO-04-0001) were used in this study.

All the experiments were conducted in the AAALAC-accrediated animal facility in compliance with the protocol approved by the Institutional Animal Care and Use Committee (IACUC).

### 3. Methods

### 3.1. Experimental Preparations

### 3.1.1. Animal Preparation

Female Balb/c nude mice, with a body weight of approximately 20 grams, were obtained from an approved vendor (Sino-British SIPPR/BK Lab. Animal Co. Ltd., Shanghai, China).

### Acclimation/Quarantine:

Upon arrival, animals were assessed as to their general health by a member of a veterinary staff or authorized personnel. Animals were acclimated for at least 3 days (upon arrival at the experiment room) before being used for the study.

### Animal Husbandry:

Animals were housed in groups during acclimation and individually housed during in-life. The animal room environment was adjusted to the following target conditions: temperature 20 to 25° C., relative humidity 40 to 70%, 12 hours artificial light and 12 hours dark. Temperature and relative humidity was monitored daily.

All animals had access to Certified Rodent Diet (Sino-British SIPPR/BK Lab. Animal Co. Ltd., Shanghai, China) ad libitum. Animals were not fasted prior to the study. Water was autoclaved before provided to the animals ad libitum. Periodic analyses of the water were performed and the results were archived at WuXi AppTec. There were no known contaminants in the diet or water which, at the levels detected expected to interfere with the purpose, conduct or outcome of the study.

### 3.1.2. Tumor Tissue Preparation

The colorectal xenograft tumor models were established from surgically resected clinical tumor samples. The first generation of the xenograft tumors in mice is termed passage 0 (P0), and so on during continual implantation in mice. The tumor tissues at passage 2 (CO-04-0002) or P3 (CO-04-0001) were used in this study.

### 3.1.3. Formulation

Test agent: high concentrated fibrinogen enriched a1at thrombin and Afod were provided by RAAS and prepared by RAAS scientist during experiment before use.

Control agent: Matrigel (BD Biosciences; cat. #356234).

### 3.2. Experimental Protocol

### 3.2.1. Establishment of Xenograft Model and Treatment

### Grouping and Treatment

Nude mice were assigned to 6 different groups with 12-17 mice/group and each group received different treatment as shown in Table 9.1.

8 out 17 (9 left) mice in high dose high concentrated fibrinogen enriched a1at thrombin and Afod group died during the first experiment using PDX model CO-04-0002. To make up for the loss of mice in high dose group, 6 additional mice were implanted with tumor fragments collected from model CO-04-0001 and treated with high dose high concentrated fibrinogen enriched a1at thrombin and Afod. So the total mice number in high dose group was 15.

TABLE 9.1
Grouping and the treatment.

| Group | Treatment | N | Remarks |
|---|---|---|---|
| 1 | Sham-operation | 12 | Open up the abdominal cavity and |
| | | | close it with sutures. (No implants) |
| 2 | Vehicle control | 12 | Implant tumor fragments of 20 mm³ |
| | | | in size into 4 corners of abdominal |
| | | | cavity. Close body with sutures. |
| 3 | Matrigel | 12 | Embed tumor fragments of 20 mm³ |
| | | | in Matrigel. Implant the tumor frag- |
| | | | ments into 4 corners of abdominal |
| | | | cavity. Close body with sutures. |
| 4 | 3 ml of high concen- | 9 + 6 | Spray high concentrated fibrinogen |
| | trated fibrinogen | | enriched a1at thrombin and Afod |
| | enriched a1at thrombin | | to cover the entire peritoneum and |
| | and Afod (high dose) | | the internal organs. Implant the |
| | on the peritoneum | | tumor fragments of 20 mm³ into 4 |
| | in abdominal cavity of | | corners of abdominal cavity. |
| | nude mice | | Close body with sutures. |
| 5 | 2 ml of high concen- | 12 | Spray high concentrated fibrinogen |
| | trated fibrinogen | | enriched a1at thrombin and Afod |
| | enriched a1at thrombin | | to cover the entire peritoneum and |
| | and Afod (moderate | | the internal organs. Implant the |
| | dose) on the perito- | | tumor fragments of 20 mm³ into 4 |
| | neum in abdominal | | corners of abdominal cavity. |
| | cavity of nude mice | | Close body with sutures. |
| 6 | 1 ml of high concen- | 13 | Spray high concentrated fibrinogen |
| | trated fibrinogen | | enriched a1at thrombin and Afod |
| | enriched alat thrombin | | to cover the entire peritoneum and |
| | and Afod (low | | the internal organs. Implant the |
| | dose) on the perito- | | tumor fragments of 20 mm³ into 4 |
| | neum in abdominal | | corners of abdominal cavity. |
| | cavity of nude mice | | Close body with sutures. |
| Total | | 76 | |

### Experiment Procedures

A. The animal was anesthetized by i.p. injection of sodium pentobarbital at 60-70 mg/kg. Disinfect the abdominal skin of nude mice with 70% ethanol solution. Open up the abdominal wall along the midline of the ventral surface to expose the peritoneal surface.
B. The surgeries for different groups were done according to table 9.1.
C. For groups using test agent, high concentrated fibrinogen enriched a1at thrombin and Afod was then applied on the peritoneal surface.
D. Tumor fragments were implanted at 4 different locations of the peritoneal cavity. The test agent acted as a glue to hold the fragments.
E. The test agent was applied again on the surface of tumor fragments and peritoneum.
F. After the fibrin membrane formed completely, the peritoneal cavity was closed.
G. In Matrigel control groups, tumor fragments were embedded into matrigel before implantation.
H. Postoperative cares followed protocol SOP-BEO-0016-1.0.
I. Mice were palpated for tumors 2 weeks after implantation. The ratio of palpable tumors observed in each group was recorded.
J. 30 days after implantation, the mice were sacrificed and tumors were dissected and weighed.
K. The tissues surrounding tumor fragments were also checked to find out whether the tumors had spread to other organ sites within the peritoneal cavity.
L. Pictures of tumor-bearing mice and dissected tumors were taken.
M. If possible, tumor sizes were measured twice per week. Tumor volumes (mm³) are obtained by using the following formula: volume=(W2×L)/2 (W, width; L, length in mm of the tumor).
N. During the experiment, health conditions of mice were observed daily. Body weights of mice were monitored twice per week.

### 3.2.2. Evaluation of the Anti-Tumor Activity

Health conditions of mice were observed daily. Body weights were measured twice per week during the treatment. Mice were palpated for tumors 2 weeks after implantation. The ratio of palpable tumors observed in each group was recorded.

30 days after treatment, all mice were euthanized with CO₂ and cervical dislocation was followed after respiratory arrest. Routine necropsy was performed to detect any abnormal signs of each internal organ with specific attention to metastases. Each tumor was removed and weighted.

### 3.3. Drugs and Materials

High concentrated fibrinogen enriched a1at thrombin and Afod were provided by

RAAS; Matrigel was from BD Biosciences (San Jose, Calif., cat. #356234). Digital caliper was from Sylvac, Switzerland.

### 3.4. Data Analysis

### 3.4.1. Relative Change of Body Weight (RCBW)

Relative change of body weight (RCBW) was calculated based on the following formula: RCBW (%)=(BWi−BW0)/BW0×100%; BWi was the body weight on the day of weighing and BW0 was the body weight before surgery.

### 3.4.2. Tumor Weight

Tumors from each mouse were pooled and weighed after sacrificing mice.

### 3.4.3. Statistical Analysis

Data were expressed as mean±SEM; the difference between the groups was analyzed for significance using one-way ANOVA and Dunnett's test.

### 4. Results

### 4.1. Tumor Growth Inhibition

Three weeks after implantation, all 12 mice in vehicle control group showed palpable tumors, while only less than 2 palpable tumors were found in each test agent-treated group.

High concentrated fibrinogen enriched a1at thrombin and Afod treatment delayed the appearance of palpable tumors as shown in table 9.2, indicating high concentrated fibrinogen enriched a1at thrombin and Afod inhibited the growth of implanted colorectal tumors in vivo.

Thirty days after implantation, tumors in vehicle control group and matrigel group reached more than 1 g on average. Conversely, tumor weights in test agent high, moderate and low dose groups were 0.49 g (0.35 if when two models are combined), 0.28 g and 0.13 g, respectively. Compared with the vehicle control, high concentrated fibrinogen enriched a1at thrombin and Afod demonstrated significant anti-tumor activities in colorectal cancer PDX model at all 3 doses. The inhibition on tumor growth were shown in FIGS. 26.18 & 26.22 and table 9.2.

### 4.2. Effect on Body Weight

Loss of body weight, a sign of toxicity, was not seen in test agent-treated groups, which only showed minor decrease in weight gain. Mortalities were observed within 3 days after surgery and treatment in high dose of test agent group, which may due to the large volume (3 ml) of test agent used in this group.

The effect on body weight was shown in FIG. 26.24 and table 9.3.

### 5. Discussion

Patient-derived colorectal tumor xenograft (PDX) model was used to evaluate the anti-cancer efficacy of the high concentrated fibrinogen enriched a1at thrombin and Afod at 3 doses. PDX tumors (CO-04-0001 and CO-04-0002) were implanted at 4 different locations in peritoneal cavity, and high concentrated fibrinogen enriched a1at thrombin and Afod, or a control agent was applied to peritoneum before and after tumor implantation.

Mice were palpated for tumors 2 weeks after implantation. The ratio of palpable tumors observed in each group was recorded. Test agent treatment inhibited the tumor growth as shown by the delayed appearance of palpable tumors. There weeks after implantation, all 12 mice in vehicle control group showed palpable tumors, while only less than 2 palpable tumors were found in each test agent-treated group (Table 9.2).

Thirty days after implantation, the mice were sacrificed and tumors were dissected and weighed. Tumors in vehicle control group and matrigel group reached more than 1 g on average. Conversely, tumor weights in test agent high, moderate and low dose groups were 0.49 g (0.35 when two models are combined), 0.28 g and 0.13 g, respectively. Compared with the vehicle control, high concentrated fibrinogen enriched a1at thrombin and Afod demonstrated significant anti-tumor activities in colorectal cancer PDX model at all 3 doses. Matrigel has been commonly used to facilitate the establishment of human tumor xenografts in rodents. In this study, matrigel group promoted an increase in tumor weight thought the increase was not statistically significant.

Loss of body weight, a sign of toxicity, was not seen in all test agent-treated groups, in which the animals only showed a minor decrease in weight gain compared to sham-operated group. Mortalities observed in test agent high dose group right after the surgery could be due to large volume of test agent (3 ml) used in this group. The mice of vehicle and matrigel groups started to loss body weights 2 weeks after surgery due to the continuously increased tumor volumes.

In summary, the results show that high concentrated fibrinogen enriched a1at thrombin and Afod at all doses significantly inhibits the growth of colorectal tumors in vivo while having minor effects on mice body weight. The results suggest that high concentrated fibrinogen enriched a1at thrombin and Afod is a potent anti-tumor agent in colorectal cancer.

### 6. References

N/A

### 7. Figures

FIG. 26.24. Anti-Tumor Efficacy of High Concentrated Fibrinogen Enriched a1at Thrombin and Afod in PDX Model CO-04-0002.
Colorectal cancer: CO-04-0002 P3

Tumor weights from model CO-04-0002 were used. Data are expressed as mean±SEM. *<0.05, ***<0.001 vs vehicle group (one-way ANOVA and Dunnett's test).

FIG. 26.22. Anti-Tumor Efficacy of High Concentrated Fibrinogen Enriched a1at Thrombin and Afod in PDX Model CO-04-0002 and CO-04-0001.
Colorectal cancer: CO-04-0002 P3+CO-04-0001 P4

Tumor weights of 6 mice from model CO-04-0001 were combined with the data from model CO-04-0002. There were 15 mice in total in high dose of test agent group. Data are expressed as mean±SEM. *<0.05, ***<0.001 vs vehicle group (one-way ANOVA and Dunnett's test).

FIG. 26.23. Photographs of Tumors Dissected from Abdominal Cavity of Each Group.

Tumors from each mouse were pooled and weighed. The tumors in frame were from model CO-04-0002 (upper panels) and the rest were form model CO-04-0001 (bottom panel). Scale bar, 1 cm.

### FIG. 26.24. Relative Change of Body Weight (%) of Different Groups.

Data are expressed as mean±SEM.

Relative change of body weight (RCBW) was calculated based on the following formula: RCBW (%)=(BWi−BW0)/BW0×100%; BWi was the body weight on the day of weighing and BW0 was the body weight before surgery.

### Confidential

### 8. Tables

TABLE 9.2
Ratios of palpable tumors observed in each group.

| | Days after surgery | | | | | | | |
|---|---|---|---|---|---|---|---|---|
| | 15 | 16 | 17 | 18 | 20 | 21 | 24 | 28 |
| Sham- | 0/12 | 0/12 | 0/12 | 0/12 | 0/12 | 0/12 | 0/12 | 0/12 |
| operated | | | | | | | | |
| group | | | | | | | | |
| vehicle | 0/12 | 1/12 | 4/12 | 4/12 | 8/12 | 12/12 | 12/12 | 12/12 |
| control | | | | | | | | |
| group | | | | | | | | |
| Matrigel | 1/12 | 3/12 | 5/12 | 5/12 | 5/12 | 8/12 | 11/12 | 12/12 |
| high dose of | 0/9 | 0/9 | 0/9 | 0/9 | 0/9 | 0/9 | 0/9 | 5/9 |
| test agent | | | | | | | | |
| moderate | 0/13 | 0/13 | 1/13 | 1/13 | 1/13 | 2/13 | 2/13 | 5/13 |
| dose of | | | | | | | | |
| test agent | | | | | | | | |
| low dose of | 0/12 | 0/12 | 1/12 | 1/12 | 1/12 | 1/12 | 2/12 | 7/12 |
| test agent | | | | | | | | |

Mice were palpated for tumors at 15, 16, 17, 18, 20, 21, 24, 28 days after implantation. The ratios of palpable tumors observed in each group were recorded.

### Confidential

TABLE 9.3
Relative change of body weight (%) of different groups.

| | | Days after | | | | | | | | | | |
|---|---|---|---|---|---|---|---|---|---|---|---|---|
| | | 0 | 1 | 2 | 3 | 4 | 5 | 6 | 7 | 8 | 15 | 45 |
| | | Group | | | | | | | | | | |
| | | RC | RC | RC | RC | RC | RC | RC | RC | RC | RC | RC |
| | | B W | B W | B W | B W | B W | B W | B W | B W | B W | B W | B W |
| Sham- | Mean | — | — | — | — | \grave{ } | 1.6 | 2.4 | 4.7 | 5.6 | 11.4 | 21 |
| operated | SD | 2.0 | 2.9 | 3.7 | 3.2 | 4.4 | 4.4 | 5.1 | 4.2 | 5.1 | 4.3 | 4.32 |
| group | SEM | 0.60 | 0.85 | 1.08 | 0.93 | 1.29 | 1.27 | 1.48 | 1.24 | 1.47 | 1.24 | 1.25 |
| Vehicle | Mean | — | — | — | — | 1.8 | 2.9 | 5.4 | 6.7 | 7.7 | 11.1 | 22.3 |
| control | SD | 0.71 | 3.19 | 2.83 | 2.41 | 3.03 | 3.03 | 3.78 | 4.18 | 4.57 | 5.56 | 6.86 |
| group | SEM | 0.20 | 0.88 | 0.78 | 0.67 | 0.84 | 0.84 | 1.05 | 1.16 | 1.27 | 1.54 | 1.90 |
| Matrigel | Mean | 0.5 | — | — | — | 1.3 | 2.3 | 5.1 | 5.7 | 6.8 | 10.8 | 23.3 |
| group | SD | 0.70 | 4.50 | 3.91 | 3.56 | 3.72 | 3.91 | 3.24 | 3.14 | 3.48 | 4.92 | 5.64 |
| | SEM | 0.19 | 1.25 | 1.08 | 0.99 | 1.03 | 1.08 | 0.90 | 0.87 | 0.96 | 1.37 | 1.56 |
| Test | Mean | 13.6 | — | — | — | — | 1.3 | 4.2 | 3.9 | 6.1 | 14.2 | 23.2 |
| agent | SD | 1.28 | 2.95 | 4.08 | 3.45 | 3.59 | 4.07 | 3.86 | 3.85 | 3.28 | 3.10 | 4.64 |
| high dose | SEM | 0.29 | 0.68 | 0.94 | 0.79 | 0.82 | 0.93 | 0.89 | 0.88 | 0.75 | 0.71 | 1.06 |
| Test | Mean | 9.7 | — | — | — | — | 0.4 | 3.2 | 5.9 | 6.2 | 10.5 | 21.9 |
| agent | SD | 0.87 | 3.06 | 3.70 | 2.82 | 3.32 | 2.82 | 3.03 | 4.07 | 2.25 | 2.65 | 4.80 |
| moderate | SEM | 0.23 | 0.82 | 0.99 | 0.75 | 0.89 | 0.75 | 0.81 | 1.09 | 0.60 | 0.71 | 1.28 |
| dose | | | | | | | | | | | | |
| Test | Mean | 2.9 | — | — | — | — | 1.7 | 4.1 | 5.2 | 5.6 | 14.5 | 26.4 |
| agent | SD | 2.88 | 2.48 | 2.73 | 3.47 | 3.97 | 3.40 | 4.03 | 3.53 | 3.69 | 4.36 | 7.15 |
| low dose | SEM | 0.80 | 0.69 | 0.76 | 0.96 | 1.10 | 1.03 | 1.22 | 1.06 | 1.11 | 1.31 | 2.15 |

| | | Days after | | | | | | | | |
|---|---|---|---|---|---|---|---|---|---|---|
| | | 19 | 22 | 26 | 29 | 33 | 36 | 40 | 43 | 45 |
| | | Group | | | | | | | | |
| | | RC | RC | RC | RC | RC | RC | RC | RC | RC |
| | | B W | B W | B W | B W | B W | B W | B W | B W | B W |
| Sham- | Mean | 15.1 | 15.3 | 16.9 | 18.6 | 19.5 | 18.3 | 21.6 | 23 | 21 |
| operated | SD | 4.1 | 4.0 | 4.5 | 4.3 | 4.4 | 3.60 | 3.4 | 3.67 | 4.32 |
| group | SEM | 1.19 | 1.17 | 1.32 | 1.25 | 1.27 | 1.04 | 0.99 | 1.06 | 1.25 |
| Vehicle | Mean | 14.4 | 14.7 | 16.2 | 17.3 | 19.7 | 18.3 | 22.5 | 23.2 | 22.3 |
| control | SD | 4.47 | 4.45 | 3.63 | 4.92 | 5.70 | 5.49 | 6.93 | 7.50 | 6.86 |
| group | SEM | 1.24 | 1.23 | 1.01 | 1.36 | 1.58 | 1.52 | 1.92 | 2.08 | 1.90 |
| Matrigel | Mean | 15.1 | 17.4 | 17.9 | 18.7 | 21.4 | 20.1 | 23.7 | 25.3 | 23.3 |
| group | SD | 5.03 | 5.55 | 4.66 | 5.92 | 6.37 | 6.68 | 5.84 | 5.28 | 5.64 |
| | SEM | 1.40 | 1.54 | 1.29 | 1.64 | 1.77 | 1.85 | 1.62 | 1.47 | 1.56 |
| Test | Mean | 16.0 | 16.6 | 18.0 | 19.0 | 21.1 | 19.2 | 23.3 | 24.6 | 23.2 |
| agent | SD | 2.77 | 3.39 | 3.42 | 3.31 | 3.63 | 4.03 | 4.08 | 4.66 | 4.64 |
| high dose | SEM | 0.64 | 0.78 | 0.78 | 0.76 | 0.83 | 0.92 | 0.94 | 1.07 | 1.06 |
| Test | Mean | 12.5 | 13.6 | 15.5 | 17.8 | 19.3 | 17.8 | 20.4 | 22.6 | 21.9 |
| agent | SD | 2.90 | 3.46 | 3.87 | 4.27 | 4.31 | 4.01 | 2.98 | 3.72 | 4.80 |
| moderate | SEM | 0.78 | 0.93 | 1.03 | 1.14 | 1.15 | 1.07 | 0.80 | 1.00 | 1.28 |
| dose | | | | | | | | | | |
| Test | Mean | 16.9 | 18.5 | 20.1 | 21.6 | 24.4 | 21.9 | 25.4 | 27.3 | 26.4 |
| agent | SD | 3.75 | 4.06 | 4.34 | 5.72 | 6.59 | 5.54 | 5.93 | 6.01 | 7.15 |
| low dose | SEM | 1.13 | 1.22 | 1.31 | 1.73 | 1.99 | 1.67 | 1.79 | 1.81 | 2.15 |

Relative change of body weight (RCBW) was calculated based on the following formula: RCBW (%)=(BWi−BW0)/BW0×100%;

BWi was the body weight on the day of weighing and BW0 was the body weight before surgery.

### Newfound GOOD HEALTHY Cells in the Existing Found Proteins and the Newly Discovered Proteins

After the inventor has discovered the method to produce the proteins containing GOOD HEALTHY CELLs-named KH CELLS.

KH CELLS are GOOD HEALTHY CELLS in which the RNA synthesizes good proteins that:

1—Send signal to the DAMAGED, SICK, AND BAD CELLS that triggers that synthesis of good proteins that transform these cells to become GOOD healthy cells.
2—Send signal to the other currently undamaged cells to synthesis of good proteins to protect them from being DAMAGED, INFECTED and PRONE to DNA and other cellular alterations.
3—Send signal to the body to produce new cells that are healthy and forbid them from being affected by intra- and extracellular damaging signals.

Thanks to this discovery the people around the world could potentially live longer healthier lives. The current population of the world as present is 7 billion people. With this discovery within the next 15 to 20 years the population could reach 10 billion people.

In order to feed such population growth the inventor discovered the process of making the medium derived from any cell to increase the protein yield for the application of the cell expression of human, animal and plant healthcare including fertilizer and maximize production of medicine, fruit, juice, meat, seafood and plants.

Fat is glucose that through the process turns into glycogen and then turns back into glucose which is a protein. The protein is within the cell to nourish the cell.

There are two kinds of cells:

### 1—A Good Healthy Cell

A good healthy cell has RNA, which produces a good protein against disease, virus, bacteria, immune deficiency and hereditary conditions in which the RNA synthesizes good proteins that 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations, 3—Send signal to the body to produce new cells that are healthy and forbid them from being affected by intra- and extracellular damaging signals.

### 2—A Bad, Damaged and Sick Cell

A bad, damaged and sick cell has RNA, which produces a bad protein that causes disease, virus, bacteria, immune deficiency and hereditary conditions.

A bad, damaged and sick cell are caused by the infiltration of the antigen such as infection, pollution, chemical, poison, radiation, hereditary condition, too much bad fat (obese) and too much sugar (diabetic).

In order to prove fat is glucose, glucose is a protein, so fat, glucose and protein are the same. The inventor has conducted testing to find out the lipid panel test for newly discovered protein plasma derived products:
All products have shown to contain High Density Lipoprotein (HDL according to TEXT BOOKS is GOOD CHOLESTEROL).
All products have shown to contain Low Density Lipoprotein/Very Low Density Lipoprotein. (LDL according to the TEXT BOOKS is BAD CHOLESTEROL)

According to the findings it is amazing all 10 products tested the level of HDL is LOWER than the LDL including APOA1 which contain only HDL (according to text books)

In order to prove Fat is a protein for the recombinant products like Factor VIII or monoclonal antibodies we have to use the fat to construct the plasmid in order to express the cell.

The life of the cell: According to the current text books the cell will die when it is exposed to alcohol or they will die by themselves in the body. There is no proof to prove the cell dies in the body.

The Cell NEVER dies, including bad cell like cancers, hepatitis and HIV.

Most tumor cells that are taken from cancer patients who died from the cancer, which have been removed for research study, these tumor cells are still ALIVE as we implant into the mice to test for tumor growth and they still grow.

In our in-vitro study for the human cell, it also can be proven that the cells are still alive in the product such as Human Albumin, Immunoglobulin, Prothrombin Complex, etc. for decades. According to the current knowledge there shouldn't be any cells in these products, because one believe that going through the process of fractionation by using 40% of alcohol, ultra-filtration at 1 micrometer and as small as 20 nanometer filtration the cell can be stripped from the protein in it.

The inventor has found that the cells are still alive and are living outside of the protein after going through further purification processes like additional alcohol, virus inactivation, pasteurization, solvent detergent, TNBP+TWIN 80, dry heating up to 100 degrees Celsius and double pasteurization.

### FIG. 26.1, 26.2

In one of our in-vitro studies for breast cancer of the nude mouse 3-7 whose tumor have been detached and we obtained that tumor and cultured the tissue and the cell appear even the tumor was out of the mouse's body.

### FIG. 26.3, 26.4

The same can be said for Animal cells which NEVER die. In our in-vitro studies, Animal meat like beef, chicken, pork, duck, seafood all have been cooked up to 100 degrees Celsius then go through our process of grinding, centrifugation and sterilized at 121 degrees Celsius for one and a half hours. When we culture these samples we have found that the cells appear.

### FIG. 26.5, 26.6, 26.7

Plants cells NEVER die. In our in-vitro studies we took lettuce, cucumber, fruits and other plants, we grinded it, centrifuge into the paste, sterilize it at 121 degrees Celsius for one and a half hours and analyzed the samples, the cells had grown up to 30 million cells instantly.

### FIG. 26.8, 26.9, 26.10

Fruit MANGOSTEEN, the cover of the Mangosteen has been used to cure the disease in the south east region, like Thailand, Malaysia, Indonesia and Vietnam. Recently this has led the scientists in the United States to initiate a research about the Mangosteen from the south East Asia region. Based upon the encouraging results of the study the business man of United States and Germany have started a joint venture to produce and introduce into the international market Mangosteen juice. The juice is rich in vitamins that help boost the immune system and can be used just like orange juice. In Vietnam people use the cover of the Mangosteen to treat diarrhea and diabetes. Now the Americans have discovered the other uses for the Mangosteen cover.

Accordingly in a human being there are thousands of free radicals always attempting to attack the normality of the cell every second of the day. All the cells in the immune system usually fight back, however when a cell loses its signal and pass through the immune system which lacks of the nutrient that causes cancers. The cancer cells, which are bad damaged cells, whose RNA has synthesized a bad protein that has sent the signal to the DNA of the good healthy cell to transform its RNA to synthesize a bad protein to become a bad damaged cell. When this happens the disease begins. Usually it will take a long time for the symptoms to show to prove that the individual is sick. This is why the diagnostic of cancers usually is too late to save the patient.

In order to support our immune system we usually use vitamin C and E. Vitamin C is very popular as it contains anti-aging properties. In our world there are a lot of anti-aging properties, among them there are two hundred strongest properties which are called Xanthones. Scientific research have found 40 Xanthones present in the cover of the Mangosteen.

In our in-vitro studies we have found the cells from the Mangosteen fruit and are doing more research on the cover and the seed.

The process to produce the medium, which can be reproduced for all other mediums such as meat, seafood, juice, fruit, etc.:

### KH101 Medium

Obtain 50 g of rice and mix it with 950 mL of water for injection and grind it to obtain the liquid form of the rice.
Centrifuge the solution to obtain the paste
Sterilize the paste by heating up to 120 degrees Celsius for 90 minutes.
Take 50 g of sterilized paste and dissolve in 950 mL of water for injection.
Grind and mix the solution for at least 15 minutes
Transfer the liquid solution into sterilized 50 cc tube.
Obtain the cell number by cell counter

Based on our method we have observed medium KH101 has reading of 20 million cells instantly by ITSELF. When to compare with our method to express CHO cell for factor VIII it will take us at least one week to reach to the 10 million cells level reading. We mixed this medium with all other blood derived products and we have observed a high increase in the cell count.

### FIG. E4 (CHO)

This method of producing this medium is to express the cell to increase the yield.

This significant cell discovery has led the inventor to believe that with this method one can also increase the protein yield of the food such as rice (KH101) from 50 g to 1,000 g (20 folds) instantly in a liquid form. In a slide with the size of 1 inch in length and ¼ inch in width, with the content of 10 microliter can contain 20 million cells. By this method the number of cells is abundant. If 50 g is considered one portion of serving, potentially can be served for 20 people. This process can be duplicated for any type of food such as drinks, protein bar, snacks, French fries. We can select the process to choose which food, fruit, meat, seafood, plant or eggs will maximize the protein cell yield. For example the giant clam has a low number of cell count to compare with others such as rice. Or egg WHITE (20 million cells) to compare with egg yoke (2 million cells).

According to textbooks the giant clam is one of the worse because of the high cholesterol. In this case we prove it has one of the lowest cell count. The reason people have problems with heart conditions or stroke as this fat will not be easy to digest and metabolize, as in our slide it has show big black particles that cannot be disintegrated even after several attempts to grind.

This will be a very amazing discovery that will help us to understand that the daily consumption of only 50 g of lettuce, 50 g of cucumber and 50 g of cherry tomatoes one will have a combination of 60 million cells.

A problem with our culture is that we do not like to eat too much vegetables or fruits, we prefer to eat meat and fat food. This is why our country has one third of the population living as obese or diabetic. By this method people can have a condensed juice, bar or pill to consume on a daily basis to maintain a healthy diet. Rather than having to eat the things that you do not like. In addition by formulating enough number of cells in each meal, like the calorie count, we can produce meals for outer space travelers or military for less weight and space for long range operations or travel.

With this kind of meal ration for the military or regular civilians we can save a lot of money in transportation and warehousing.

With this discovery one also can mass produce the protein to naturally protect any crop from any infiltrating antigen delivered by insects, animals or other source.

With this discovery it can help to manufacture a powerful fertilizer containing the protein or urea. Where the fertilizer can be supplied in a small bar to be dissolved in water in order to fertilize for 1 hectare, whereas 1 ton of urea has to be used. With this discovery also it can help save the bulky transportation of fertilizer.

With this discovery one can increase the crop yield of each individual plant by supplying enough protein and nutrients to multiply the number of fruits, soy bean, rice, nuts, etc. from the same amount of plants.

We also found a very interesting thing that the KH101, which has a very high concentration can block the cancer cell. We also found that for a small grape, whose number of genes, is around 30,000. While a person with 70 kilos has only 25,000 genes. This grape medium also interfere with lung cancer cells and further investigations are still ongoing. For this reason the inventor believes the theory of bad fat and bad food cause problems for anybody, and therefore a good protein with a high number of good cells can help anybody, just like diet.

Description of the KH mediums:
KH101 consist of 50 g of rice paste sterilized at 120 degrees Celsius for 90 minutes in 1 liter of water for injection. Tryptophan is added as a stabilizer.

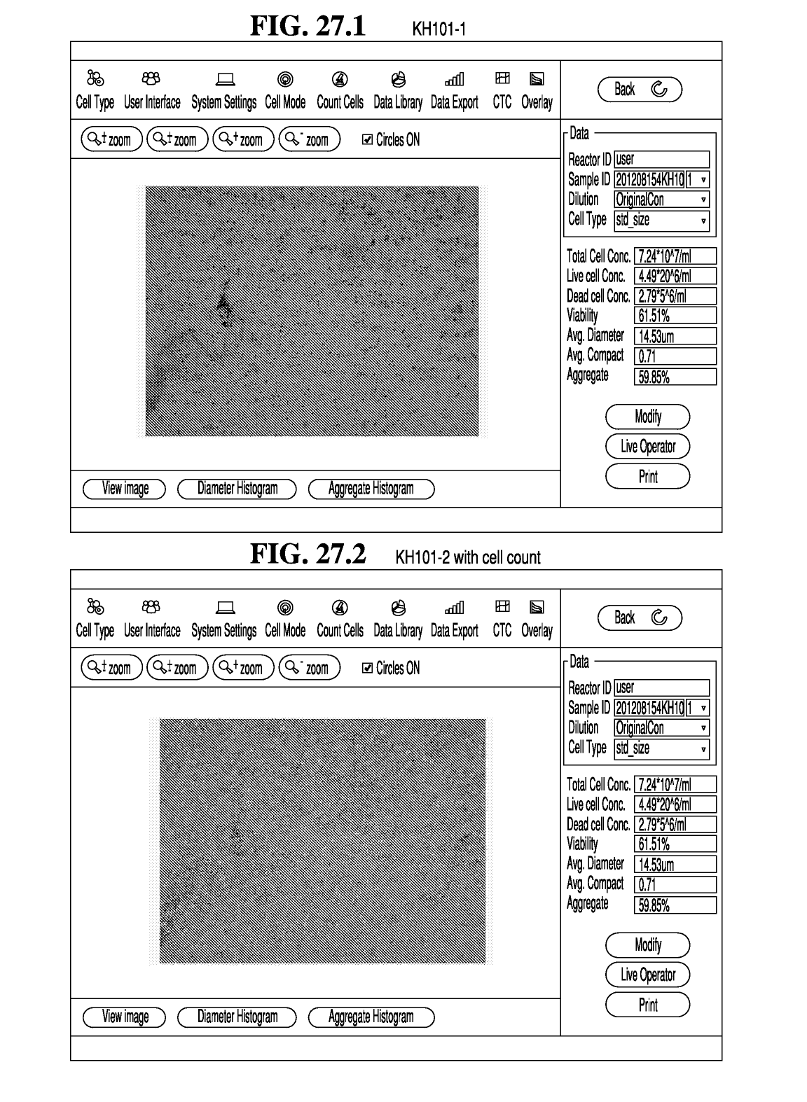

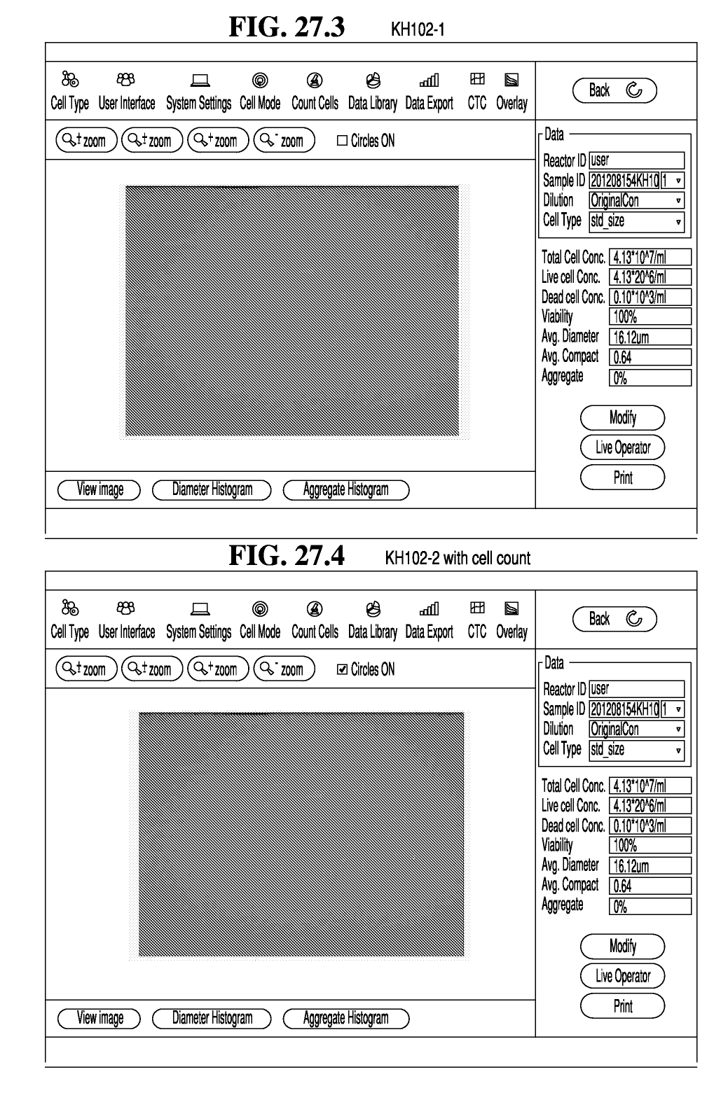

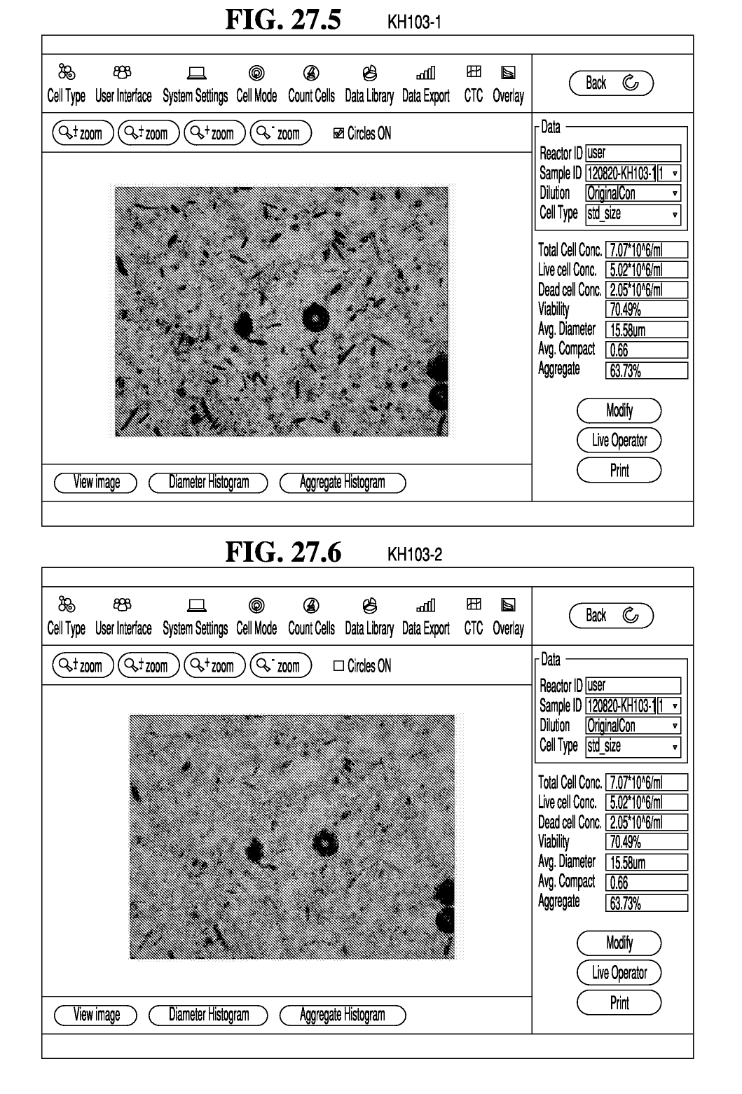

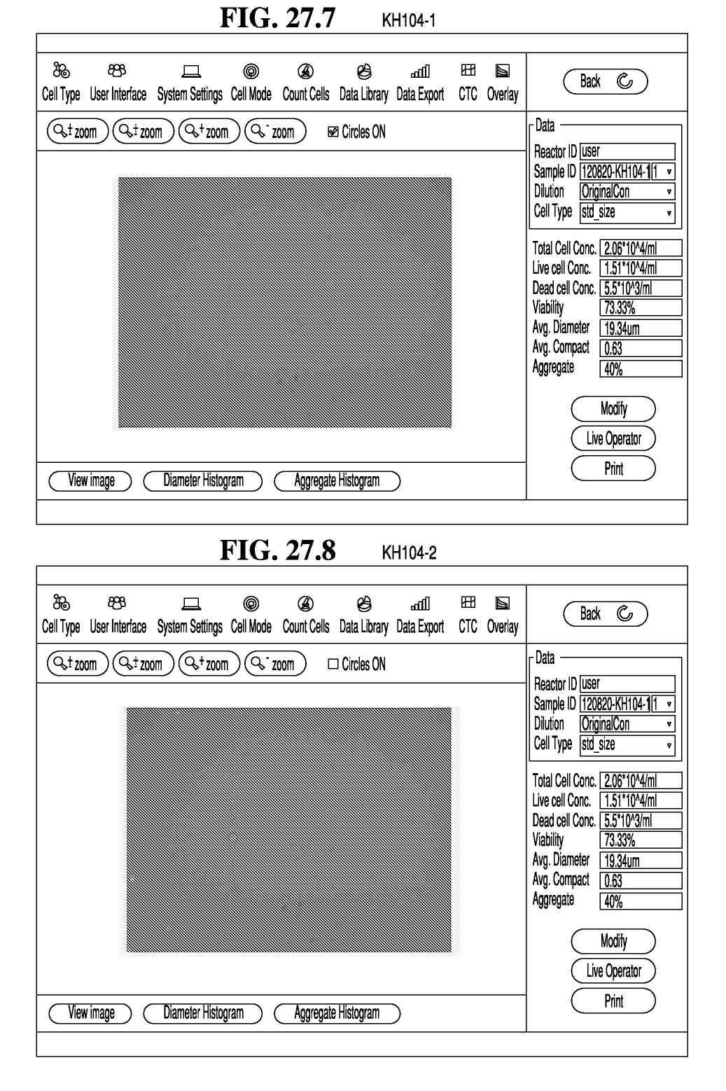

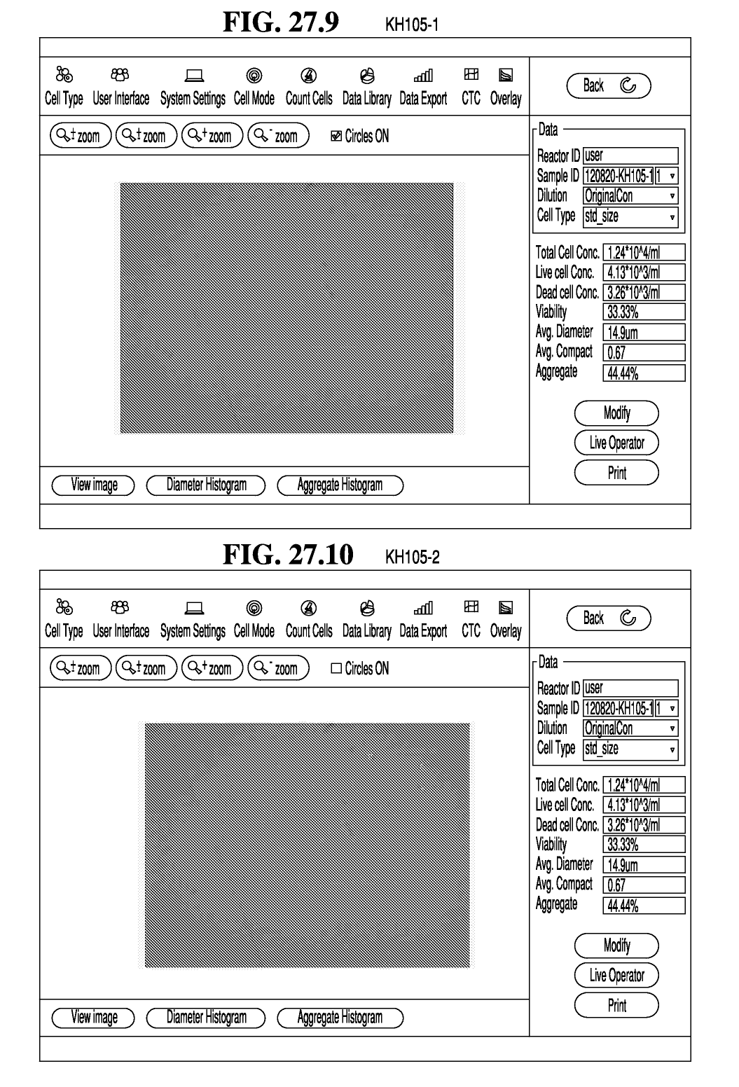

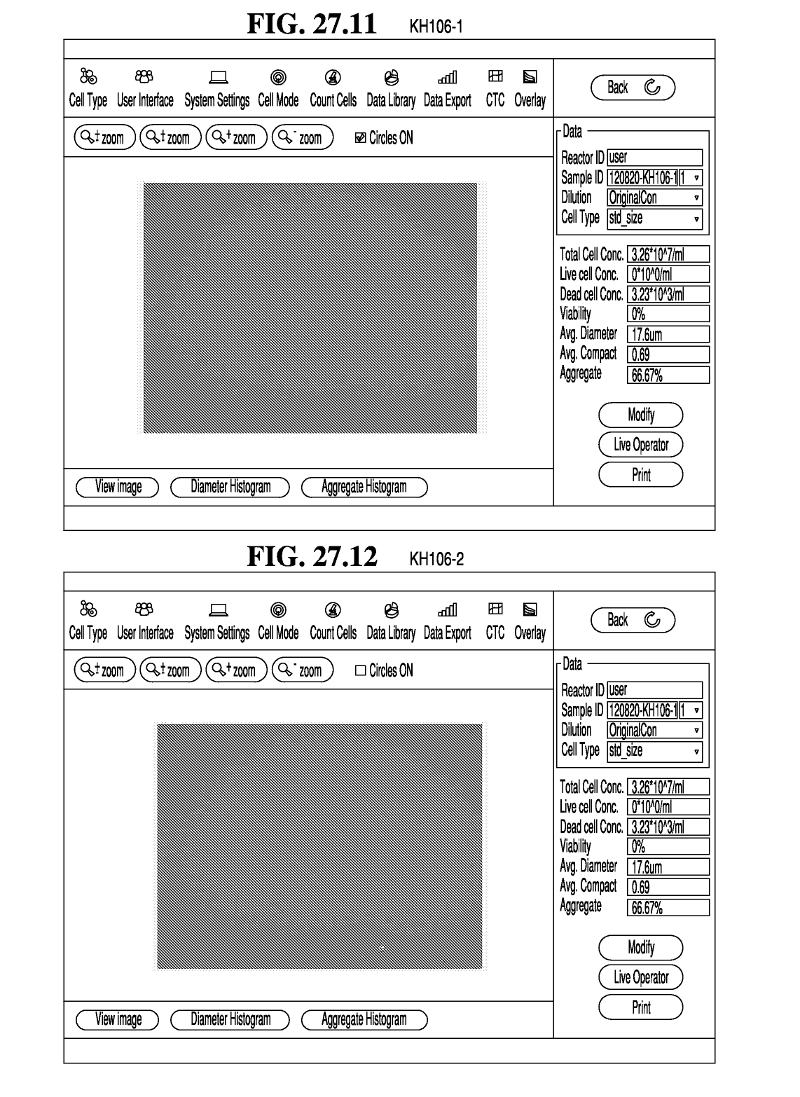

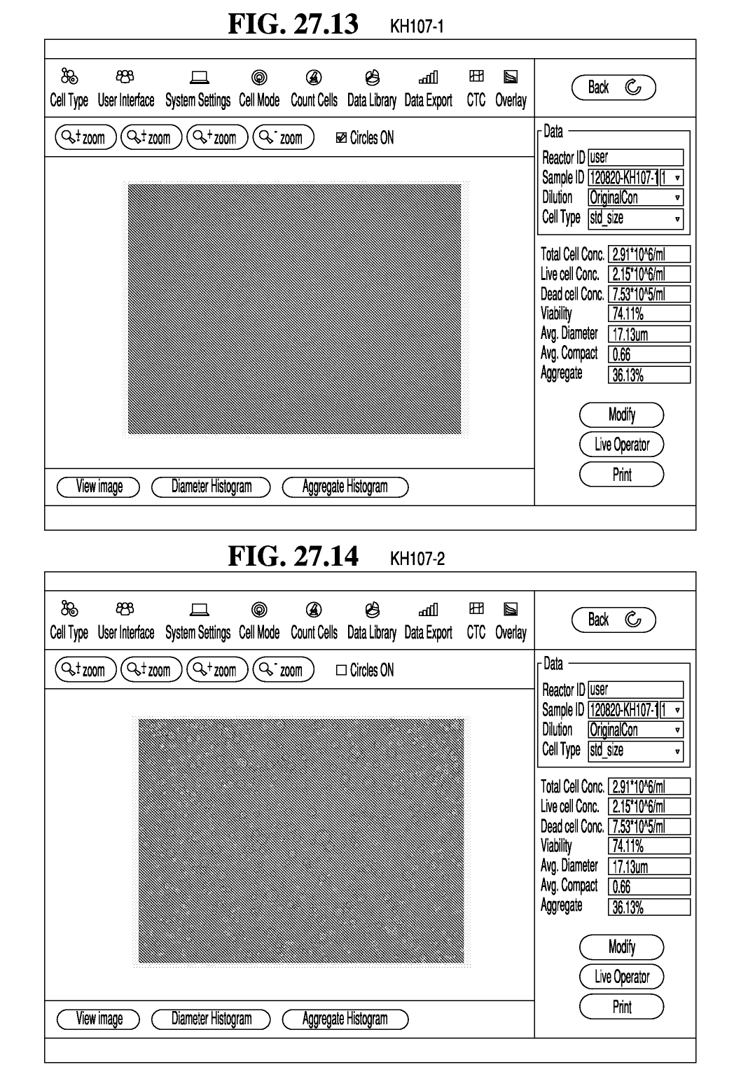

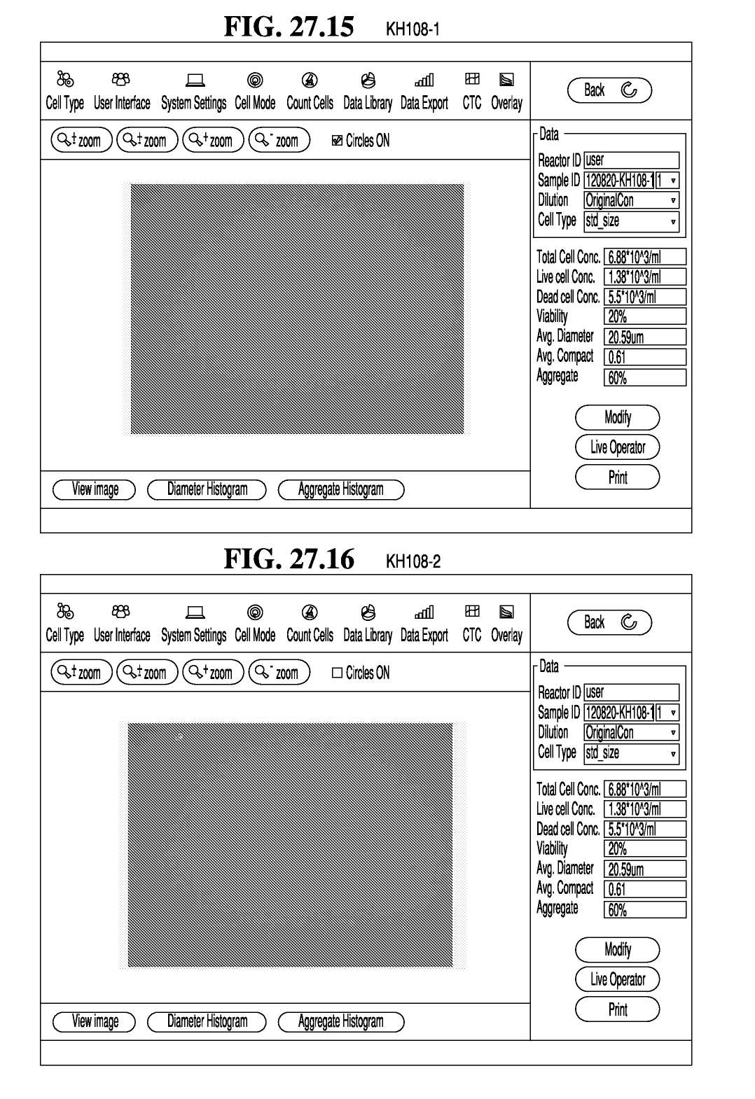

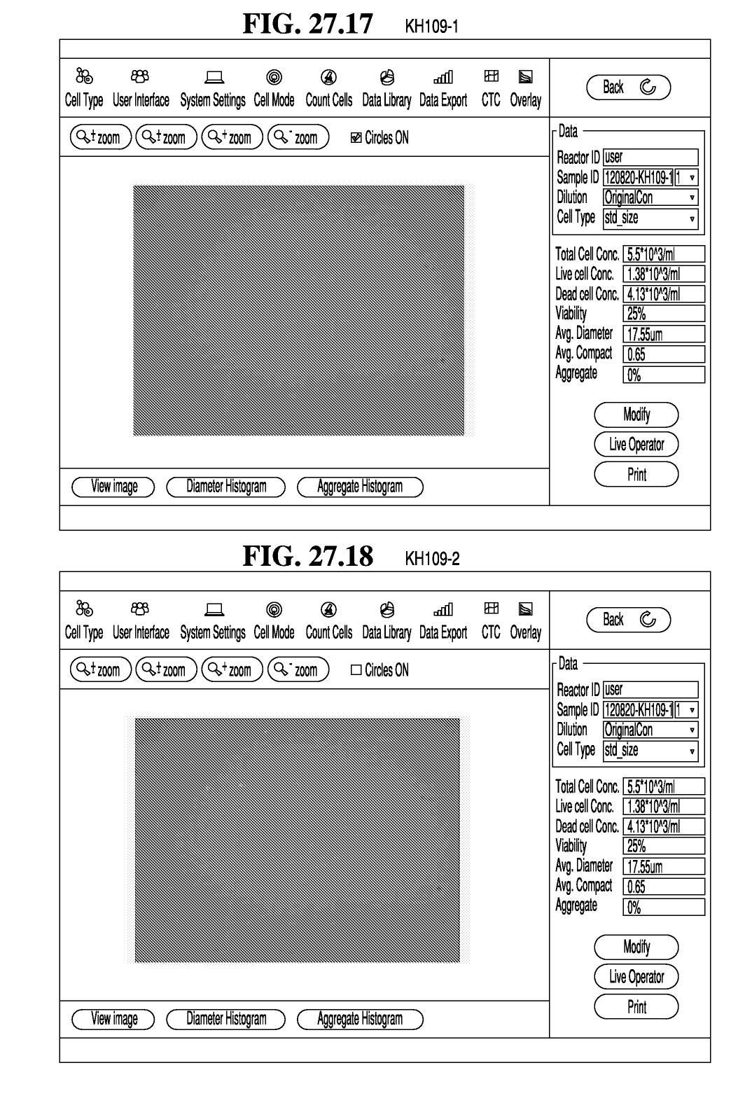

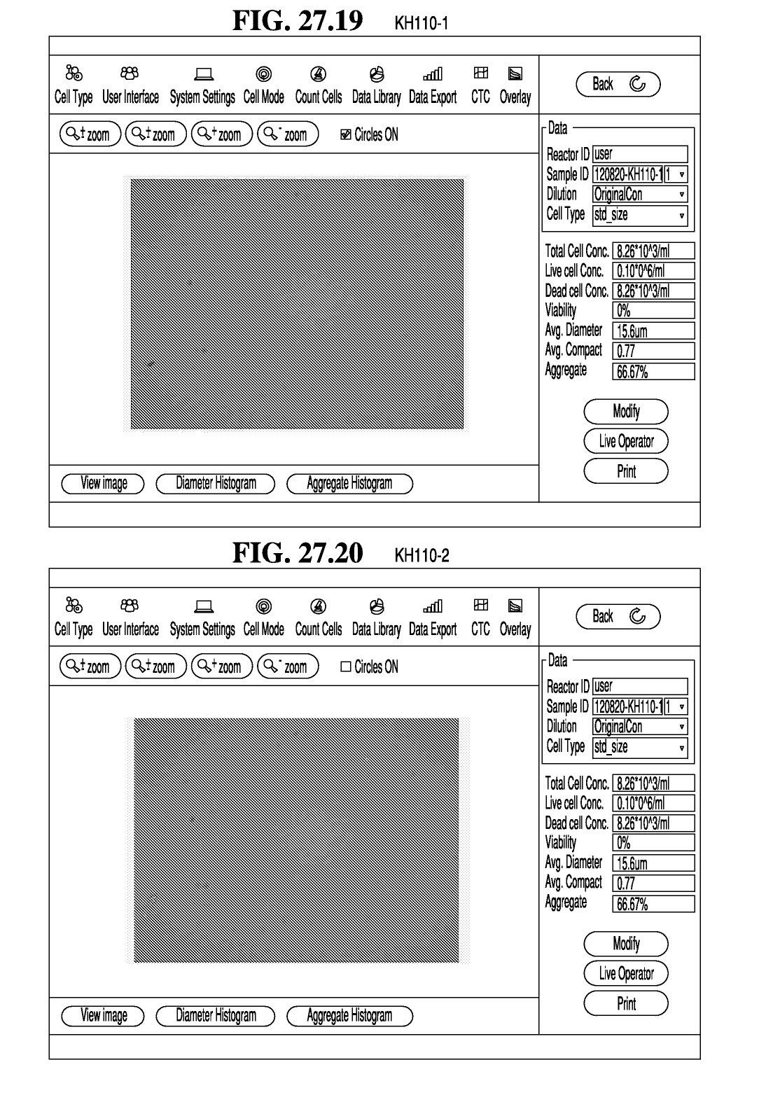

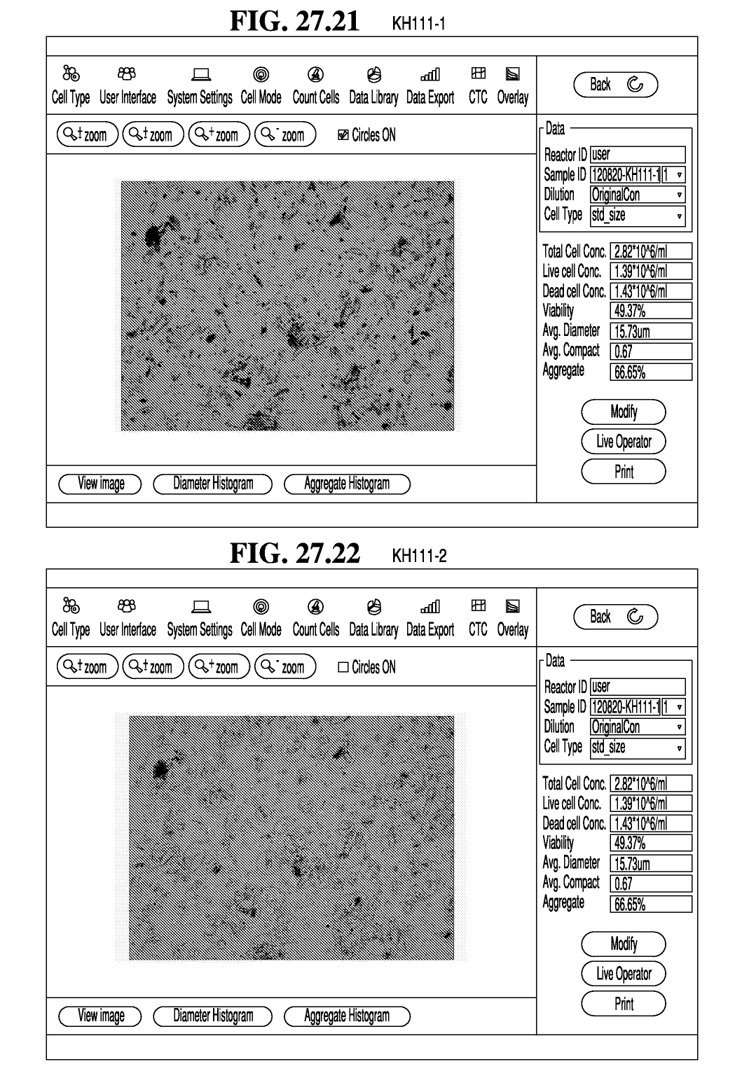

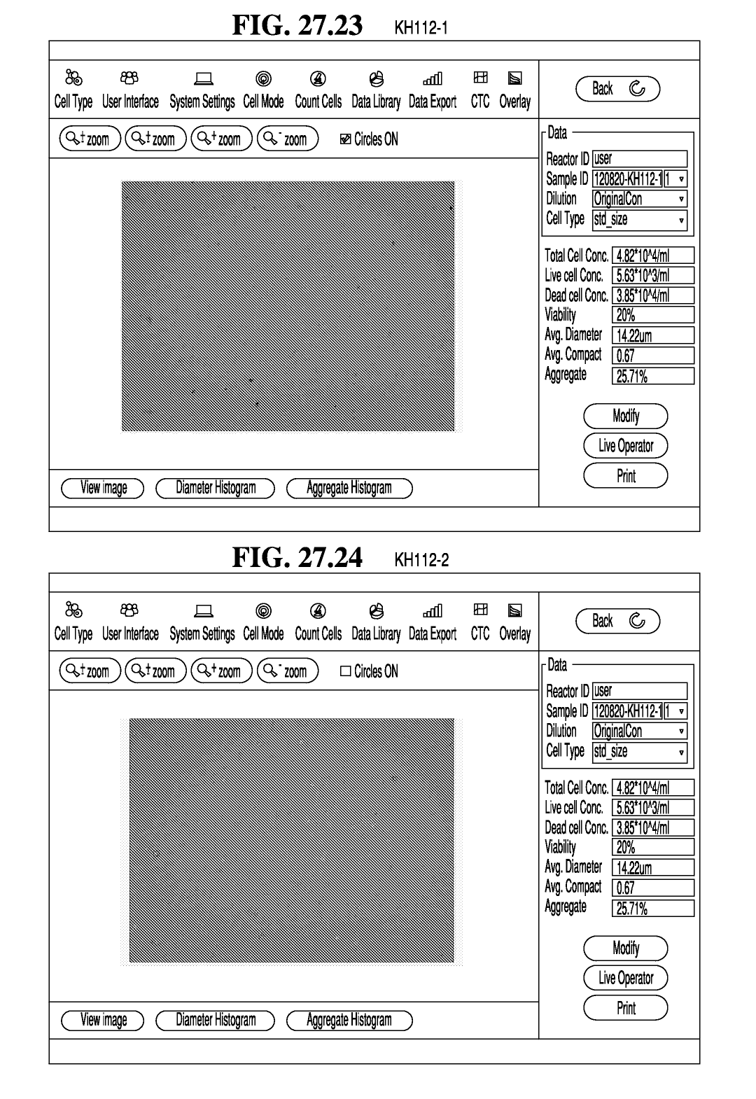

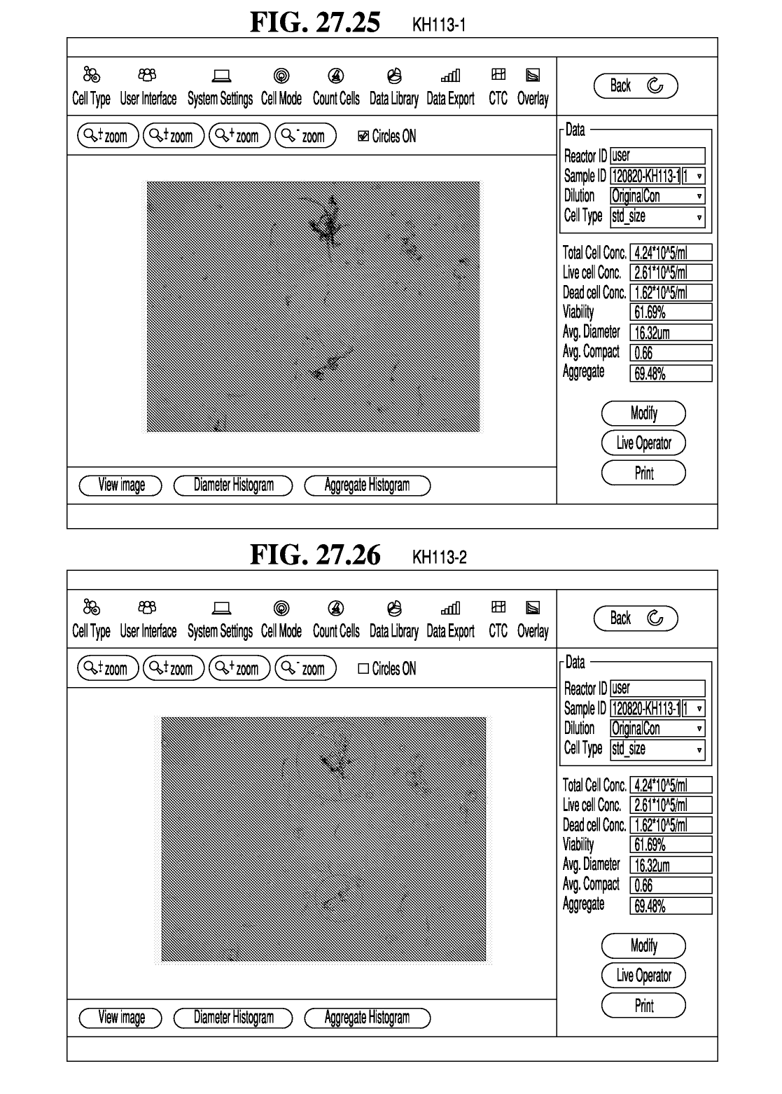

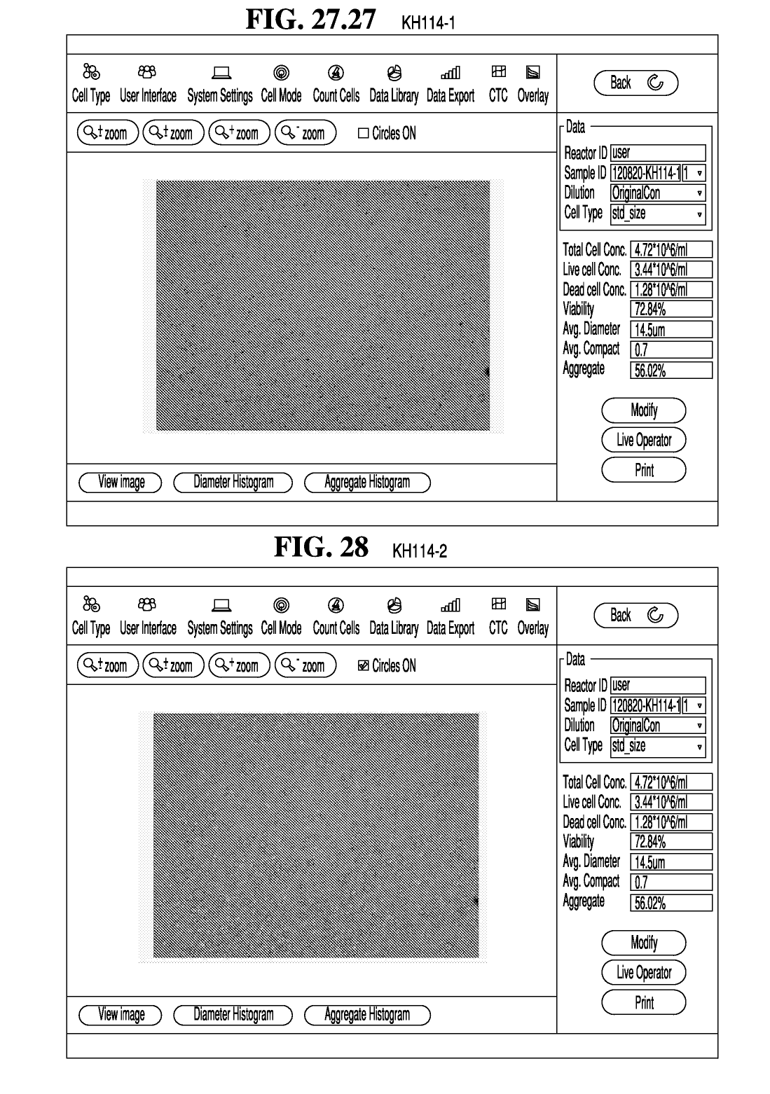

FIGS. 27.1 and 27.—KH102 consists of Urine.

FIGS. 27.3 and 27.—KH103 consists of 50 g paste of Soybean into 1 liter of water for injection.

FIGS. 27.5 and 27.—KH104 consists of 50 g paste of Orange juice into 250 mL of water for injection.

FIGS. 27.7 and 27.—KH105 consists of 30 g of paste of Grape juice into 500 mL of water for injection.

FIGS. 27.9 and 27.1—KH106 consists of 23 g of paste of Apple juice into 500 mL of water for injection.

FIGS. 27.11 and 27.1—KH107 consists 50 g of paste of Sticky Rice into 1000 mL of water for injection.

FIGS. 27.13 and 27.1—KH108 consists of water for injection

FIGS. 27.15 and 27.1—KH109 consists of white wine with 13% alcohol level.

FIGS. 27.17 and 27.1—KH110 consists of red wine with 14% alcohol level

FIGS. 27.19 and 27.2—KH111 consists of 50 g of paste of Green Bean into 1000 mL of water for injection.

FIGS. 27.21 and 27.2—KH112 consists of 50 g of paste of Oat into 1000 mL of water for injection.

FIGS. 27.23 and 27.2—KH113 consists of 50 g of paste of Chestnut into 1000 mL of water for injection.

FIGS. 27.25 and 27.2—KH114 consists of 50 g of paste of Dorian fruit into 1000 mL of water for injection.

FIG. 27.27 and FIG. 2—KH115 consists of 23 g of paste of Raspberry into 450 mL of water for injection.

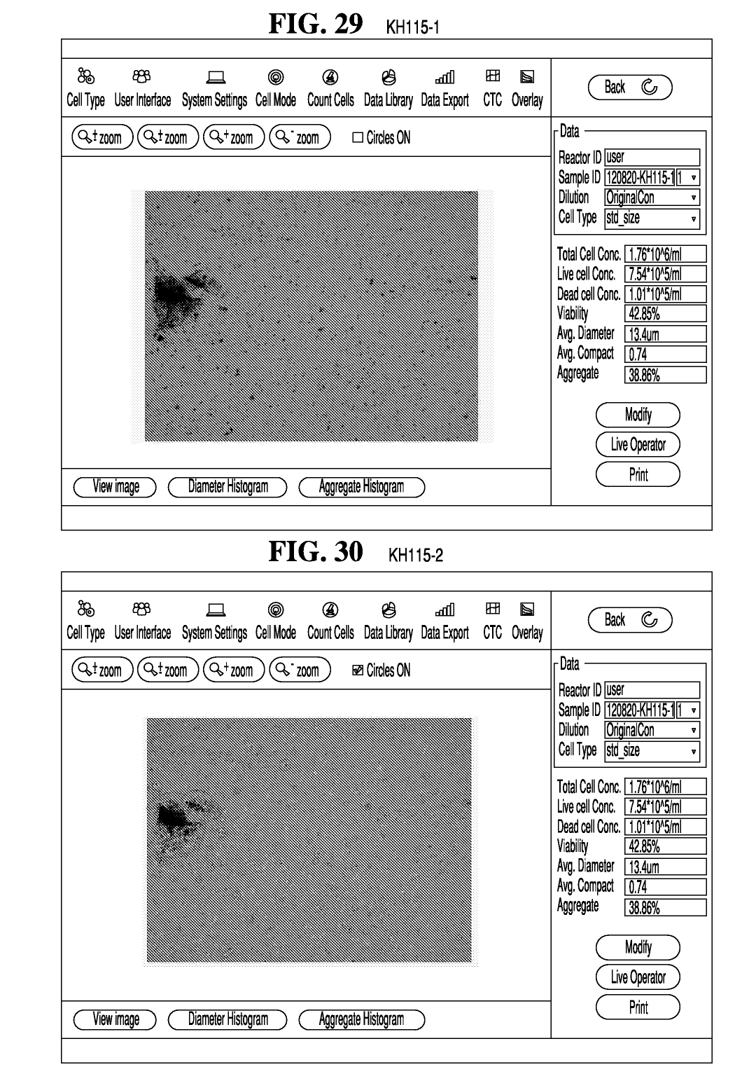

FIGS. 29 and 3—KH116 consists of 23 g of paste of Pear into 400 mL of water for injection.

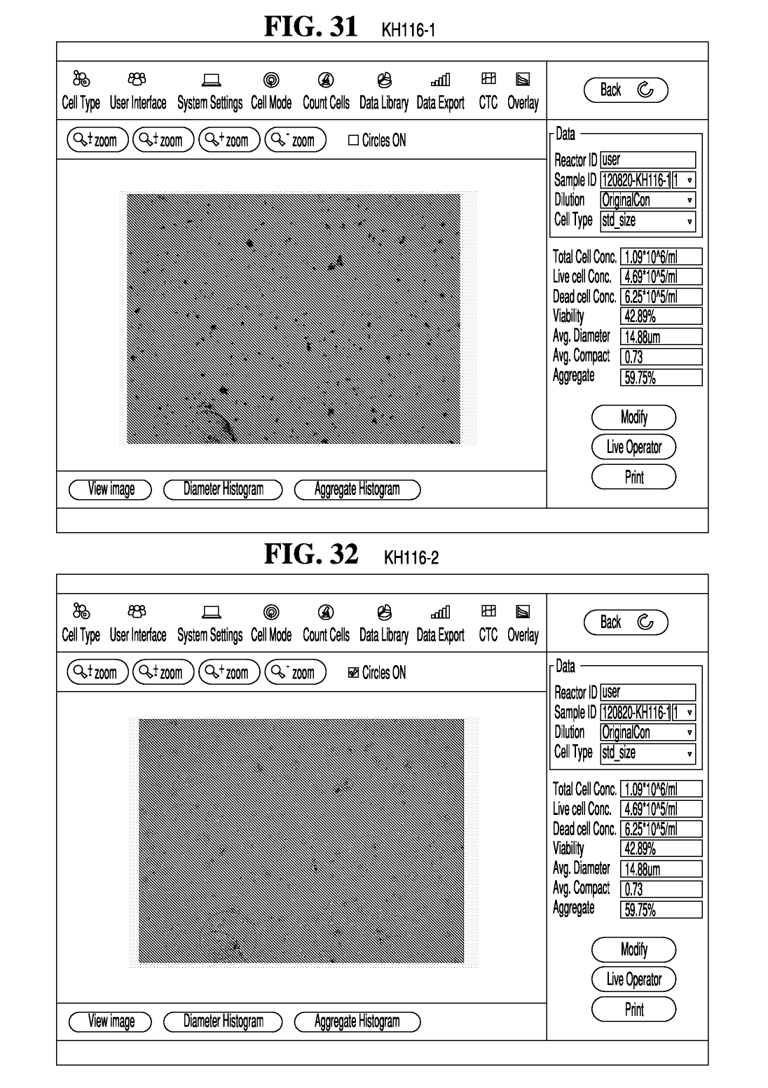

FIGS. 31 and 3—KH117 consists of 50 g of paste of Jack Fruit into 1000 mL of water for injection.

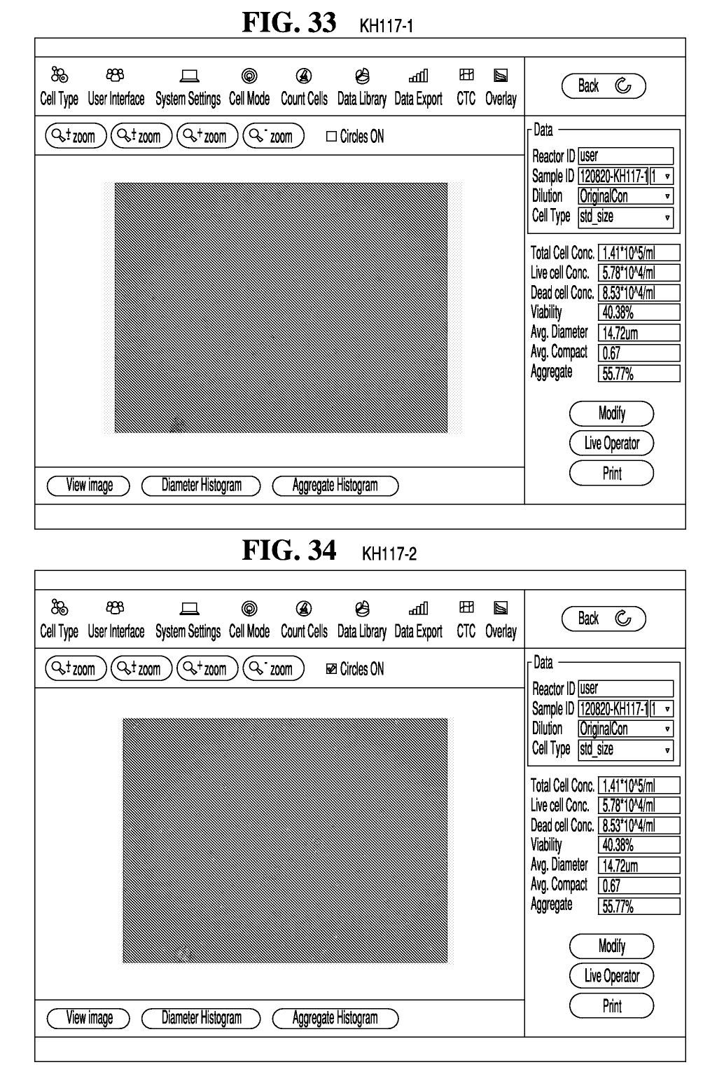

FIGS. 33 and 3—KH118 consists of 32 g of paste of Water Apple into 600 mL of water for injection.

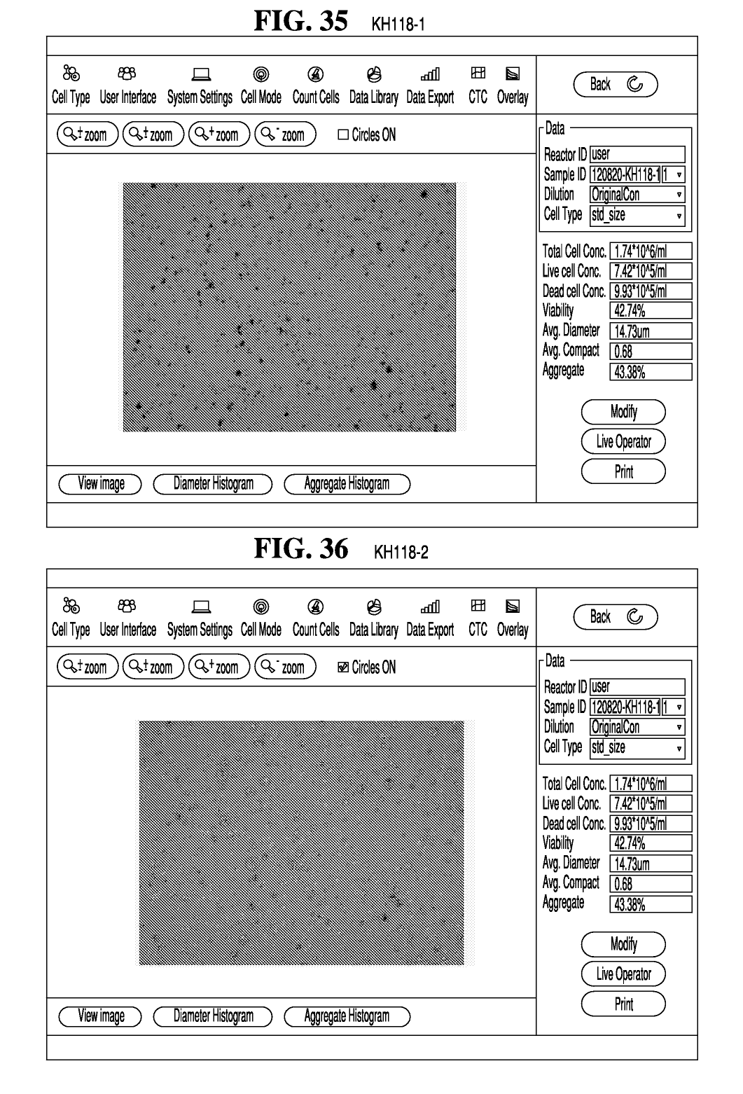

FIGS. 35 and 3—KH119 consists of 52 g of paste of Mangostine into 1000 mL of water for injection.

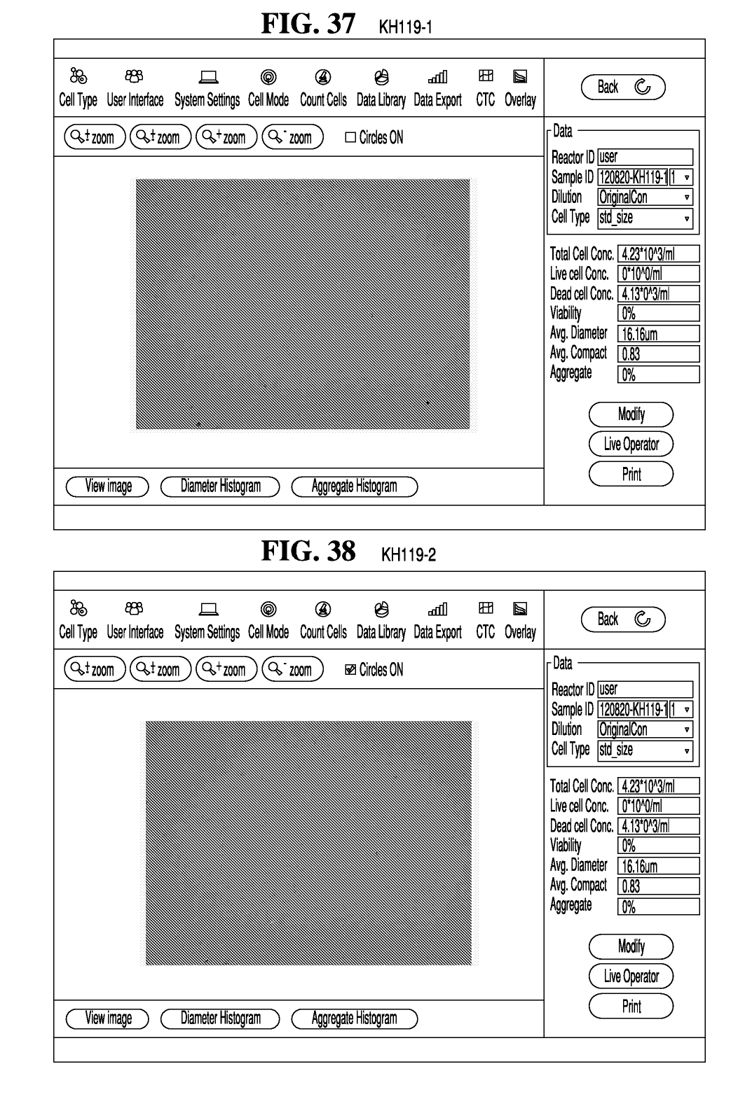

FIGS. 37 and 3—KH120 consists of 10 g of paste of Lettuce into 20 mL of water for injection.

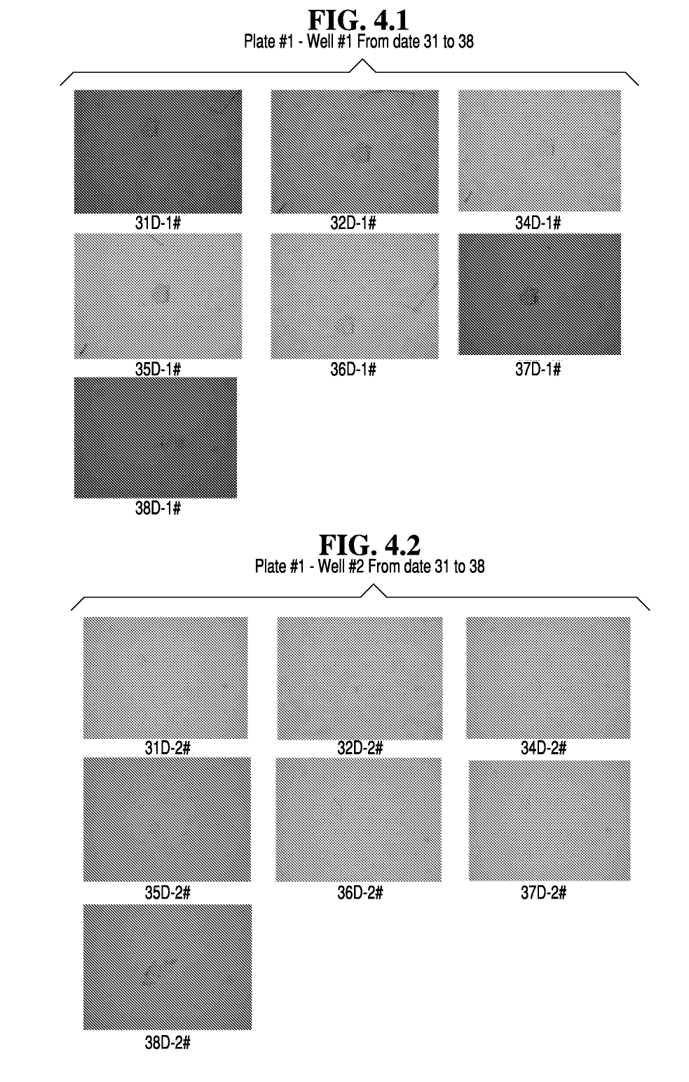

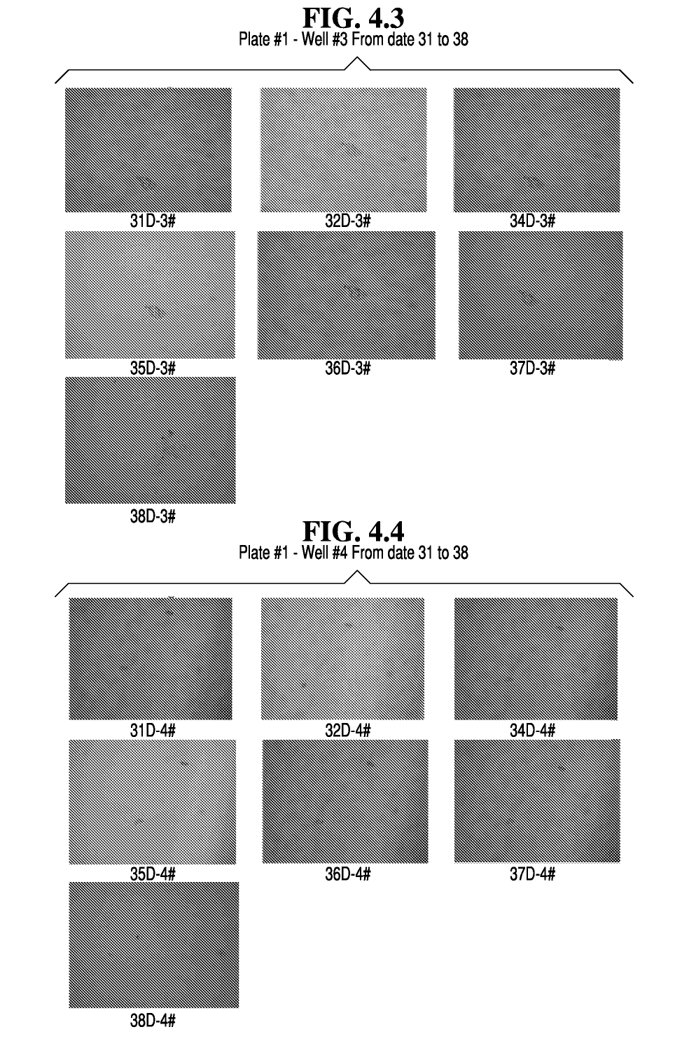

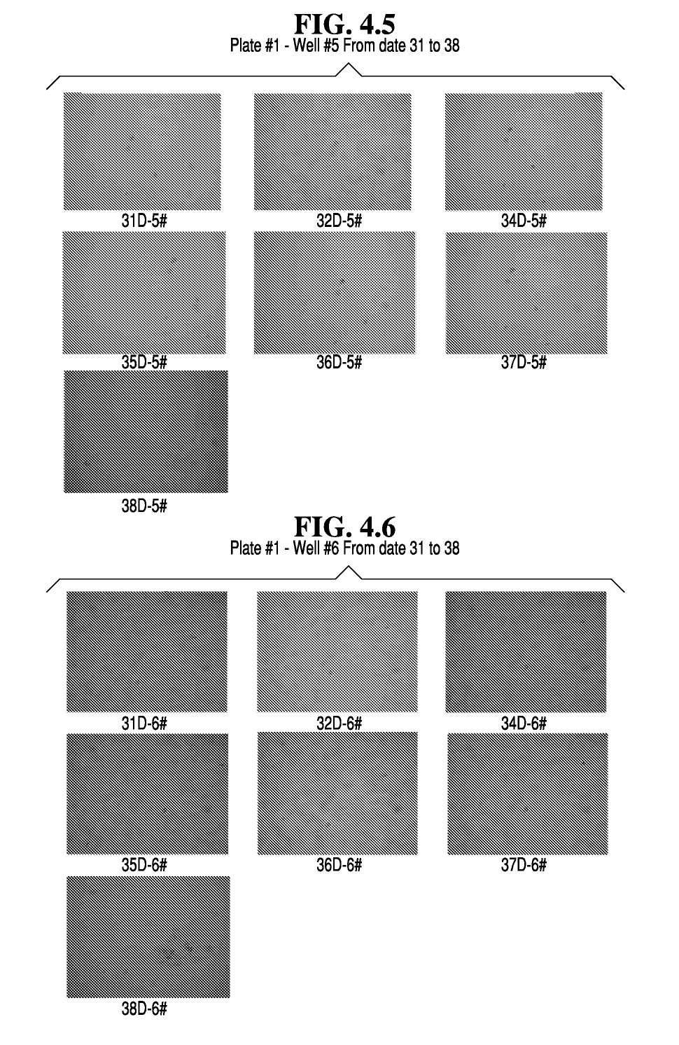

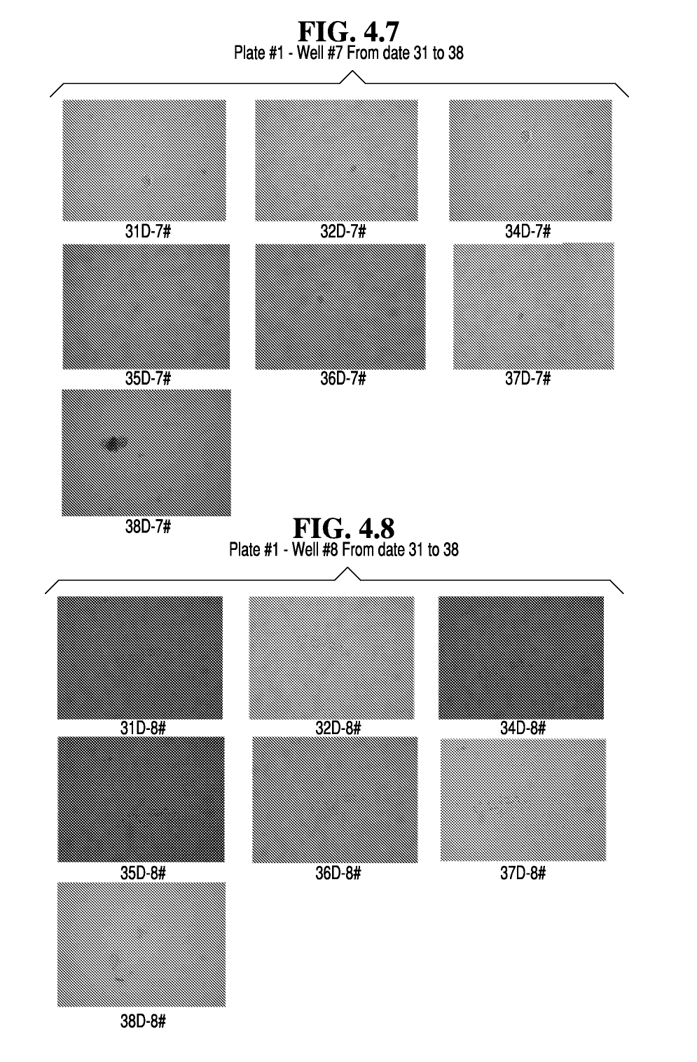

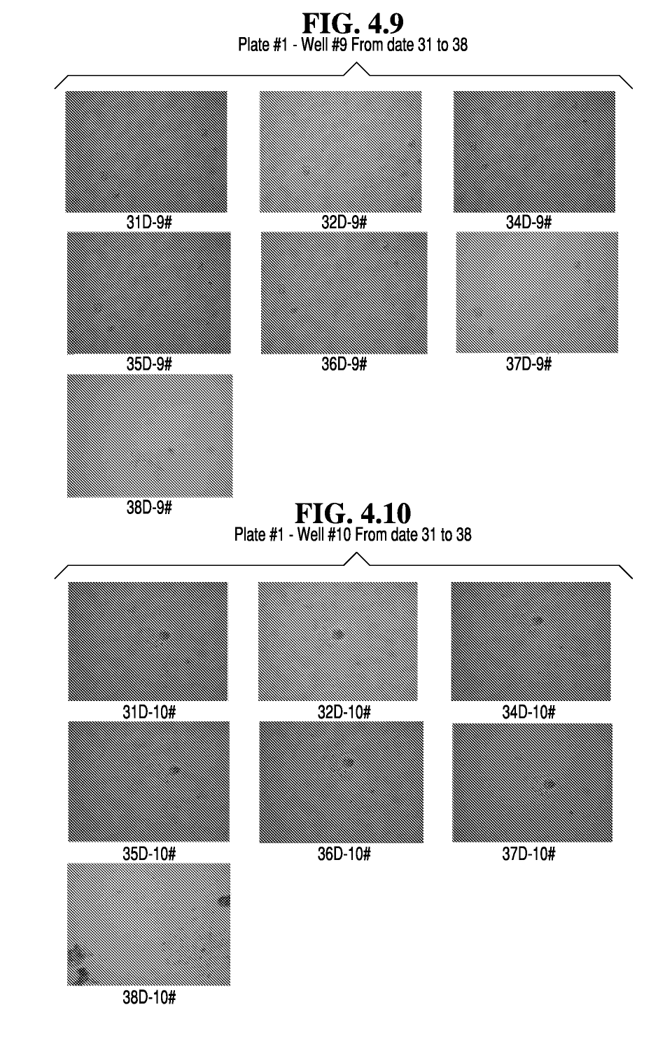

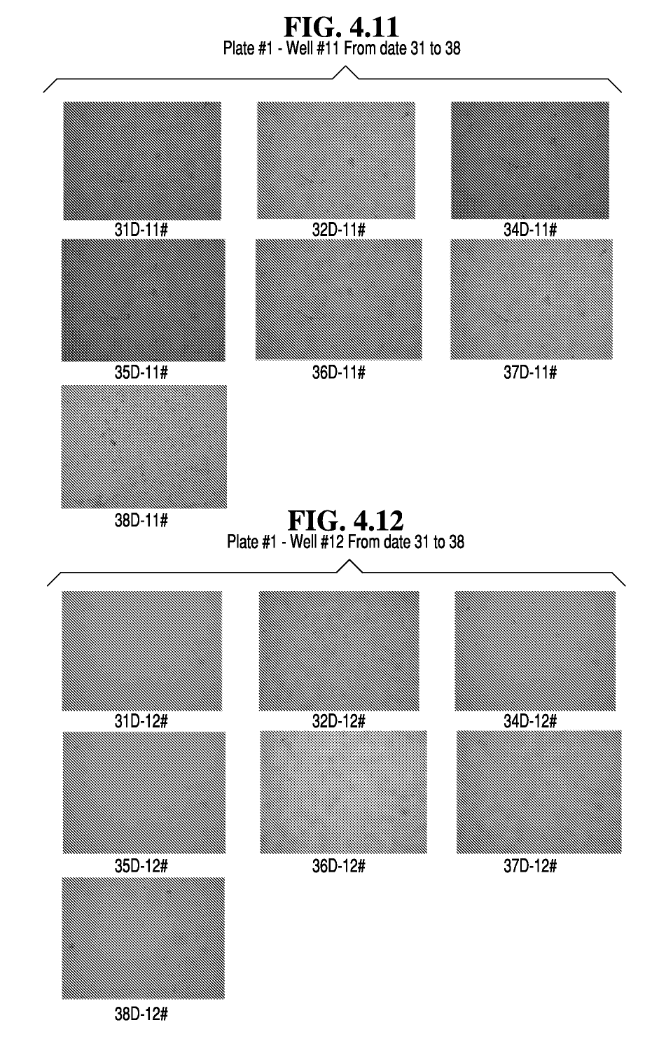

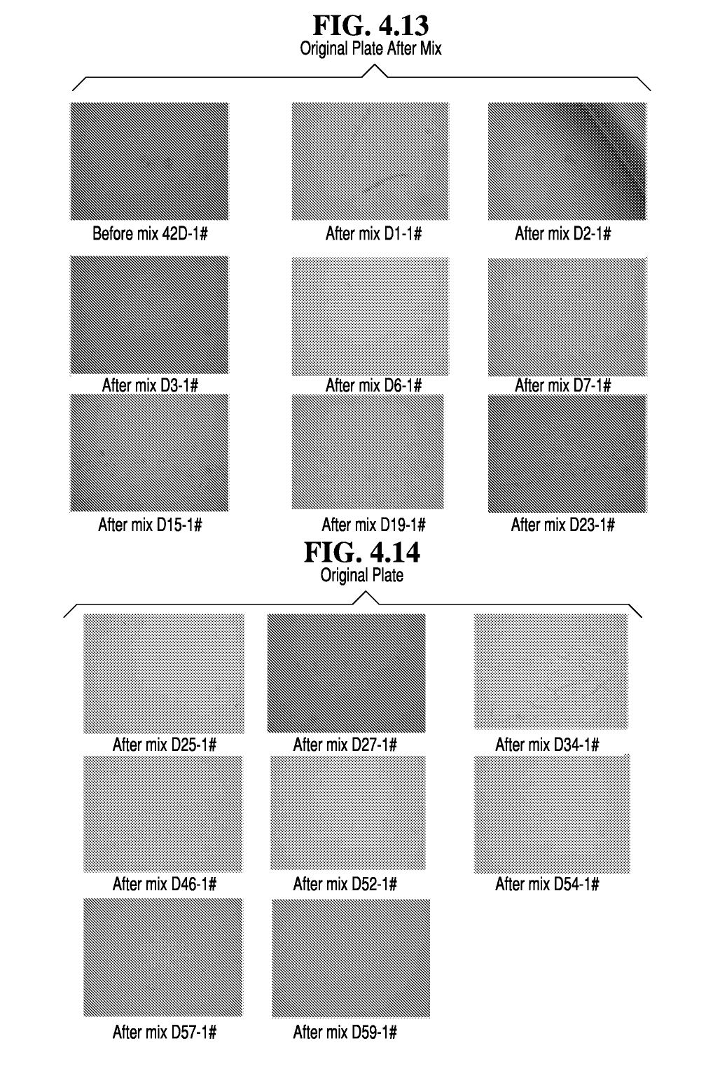

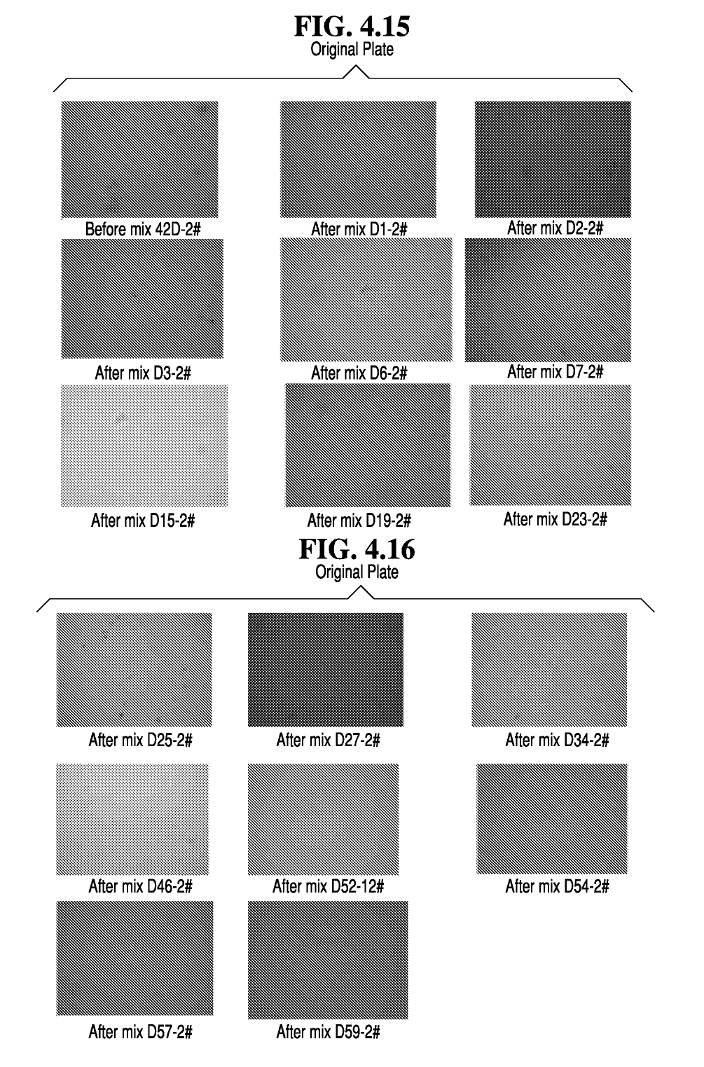

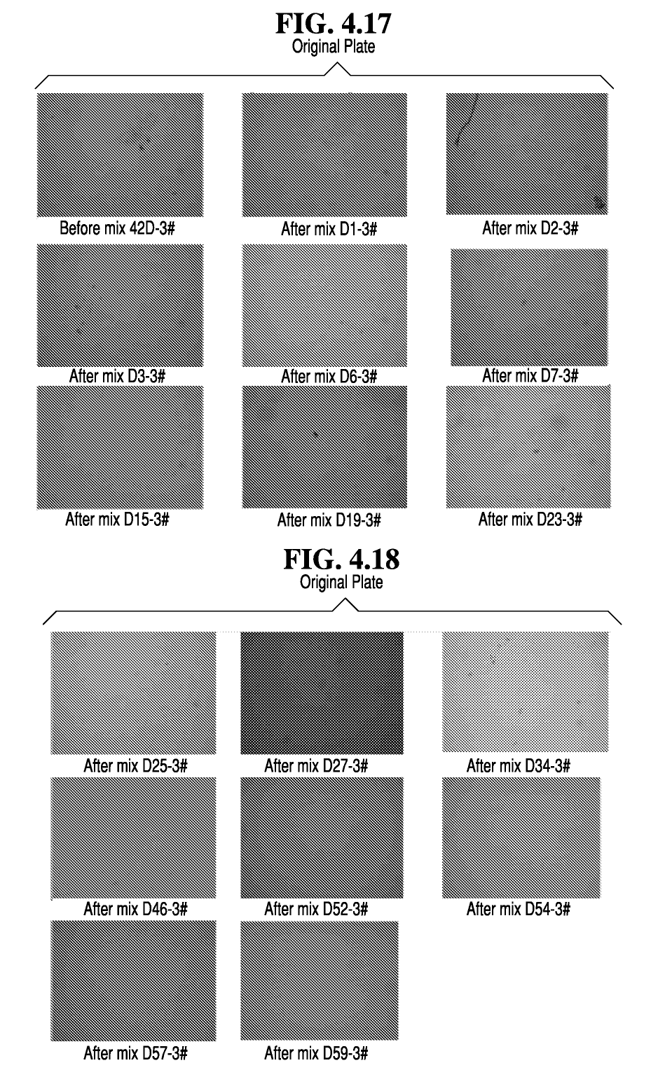

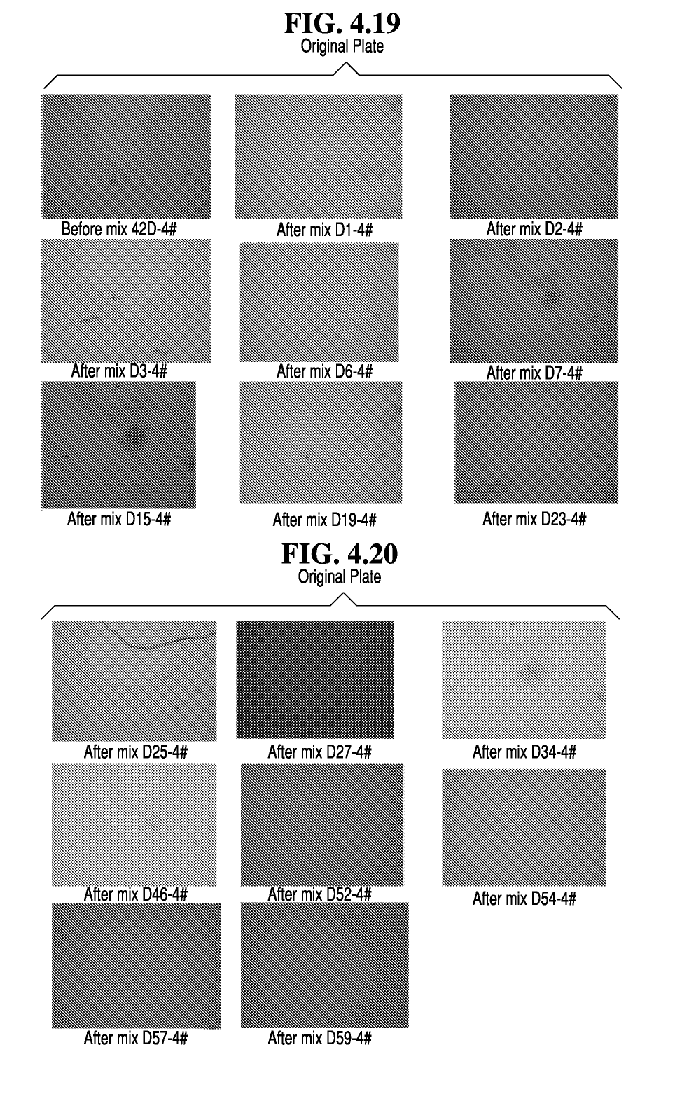

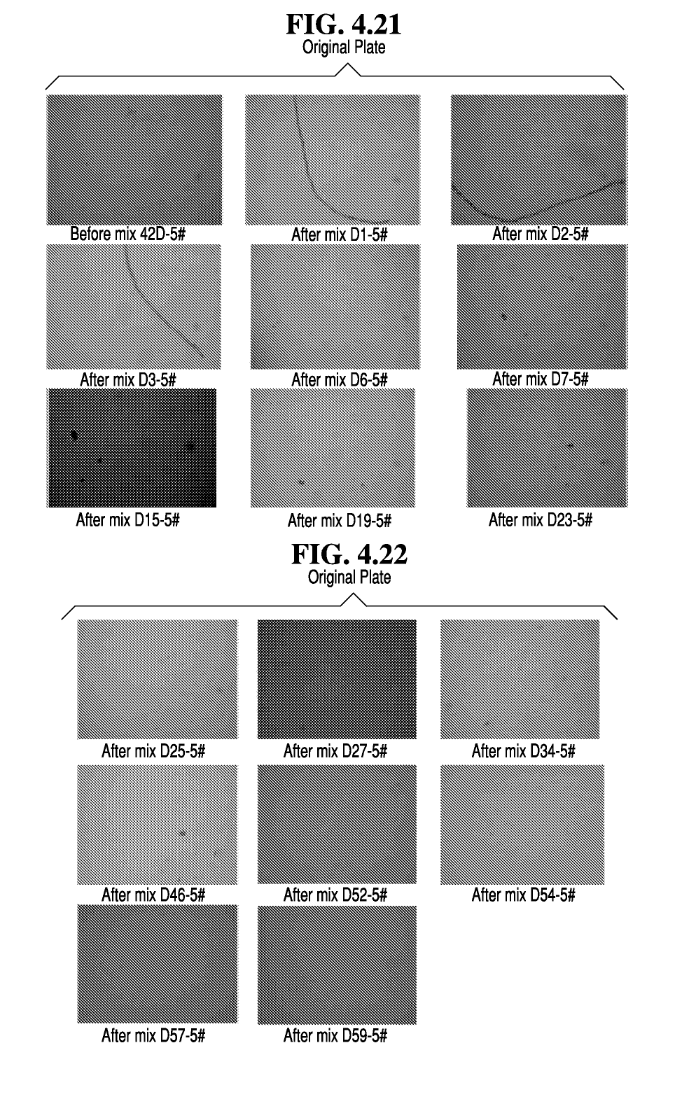

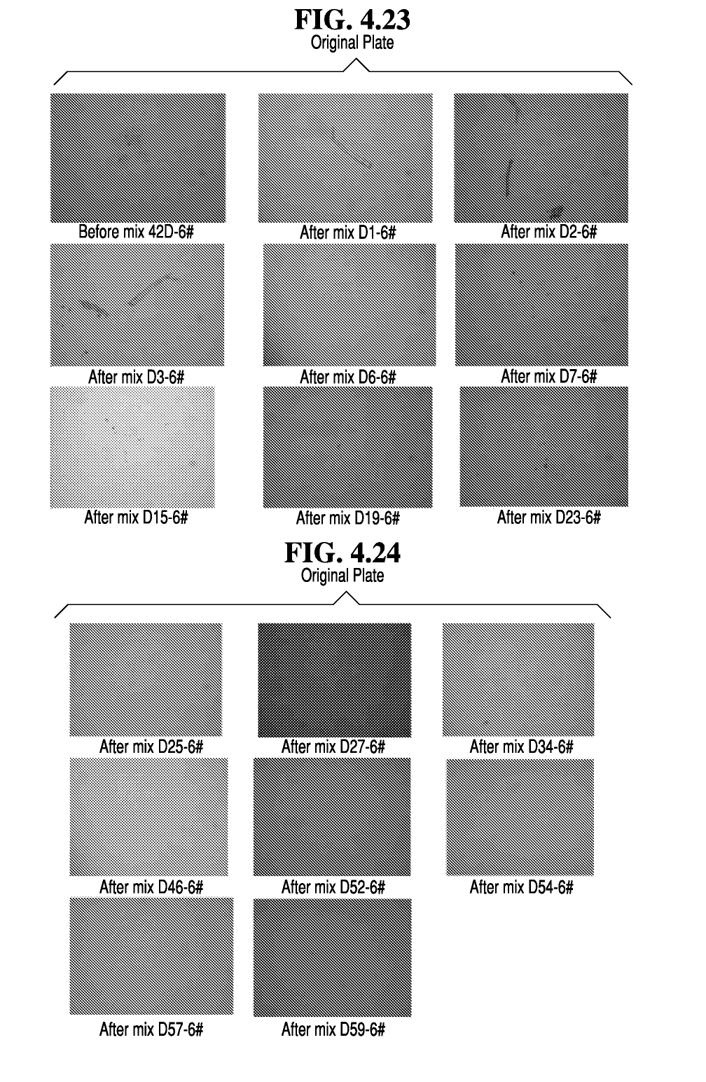

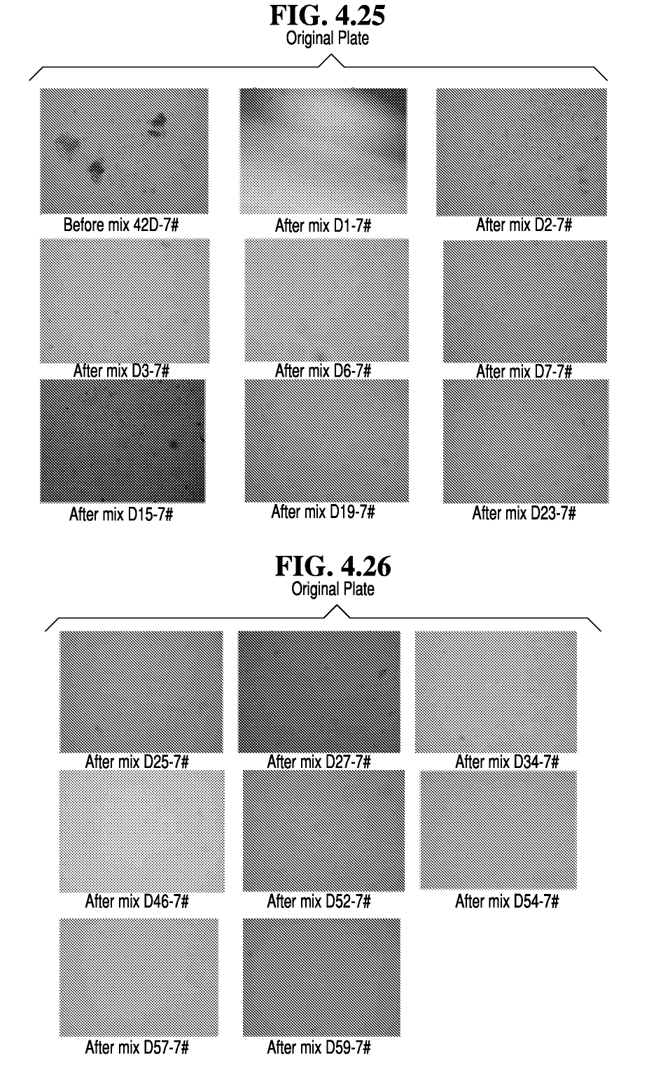

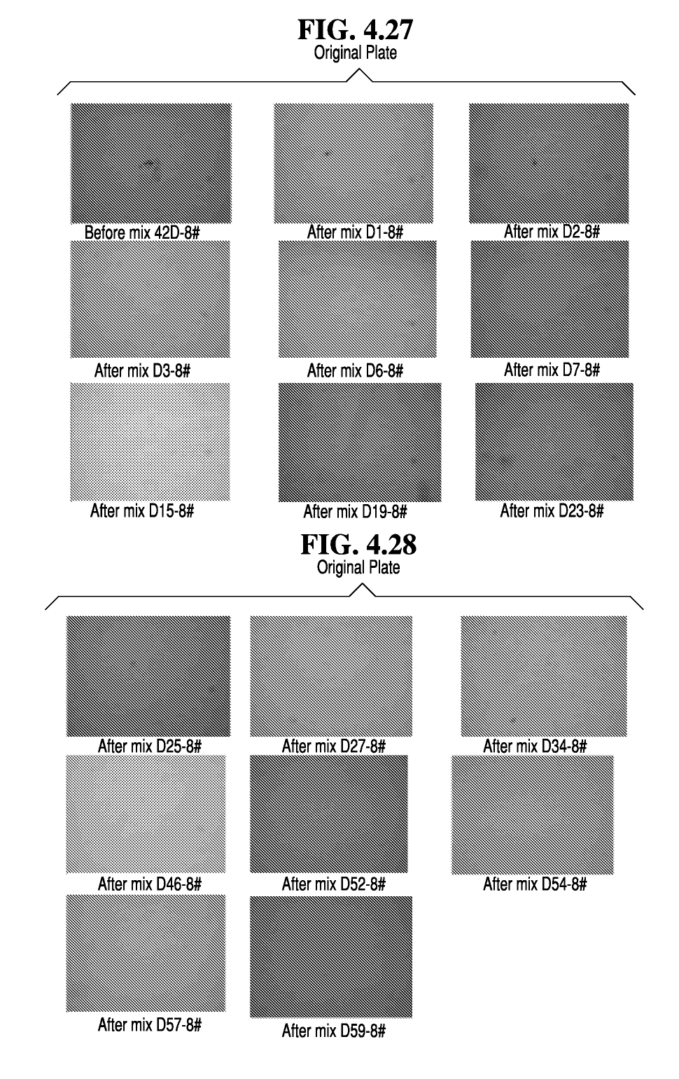

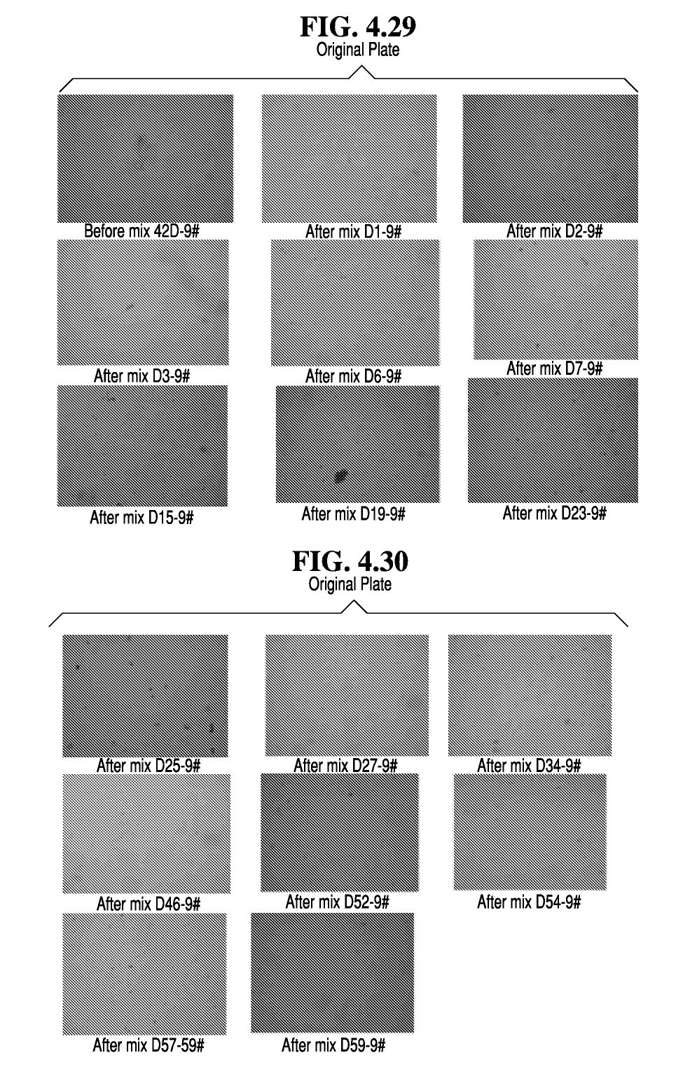

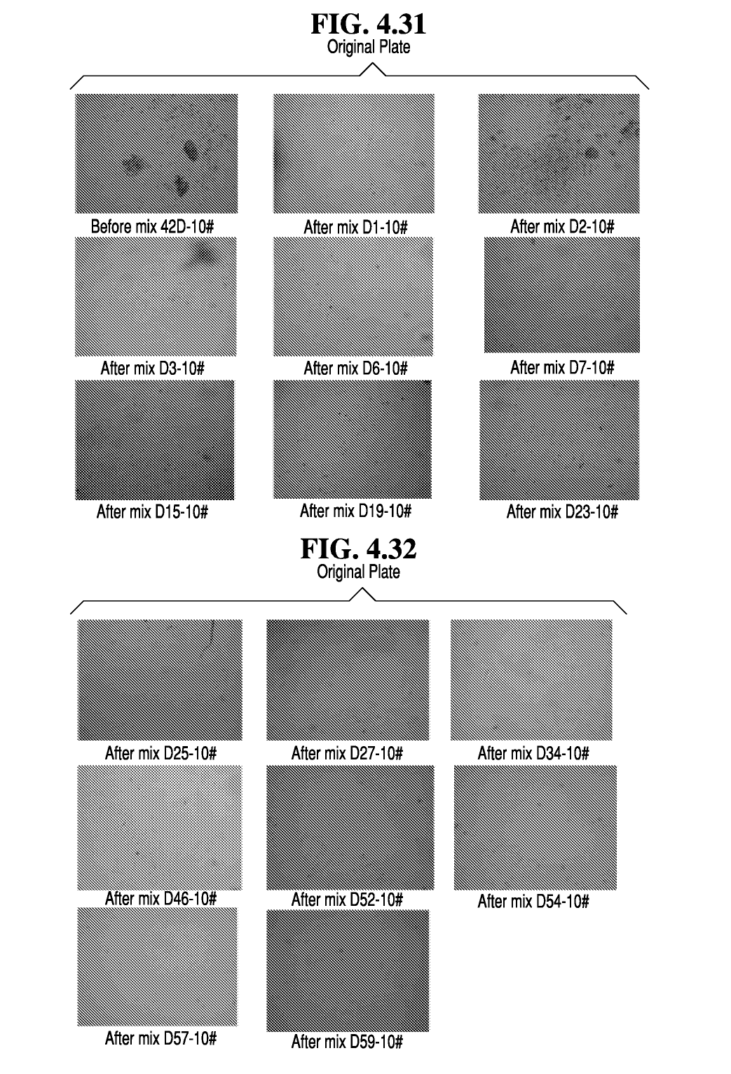

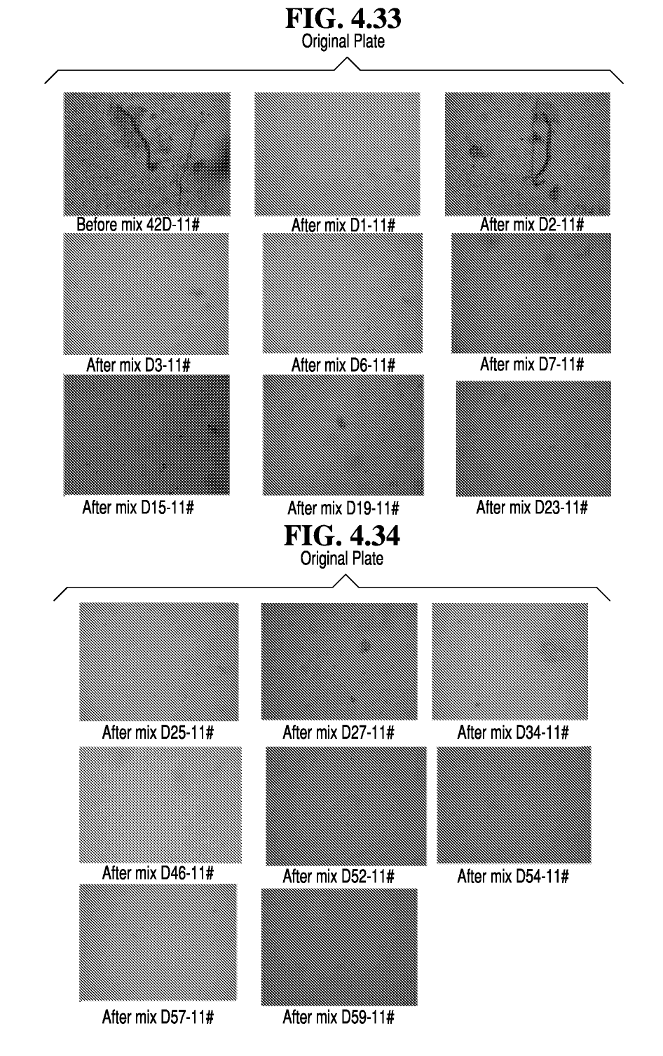

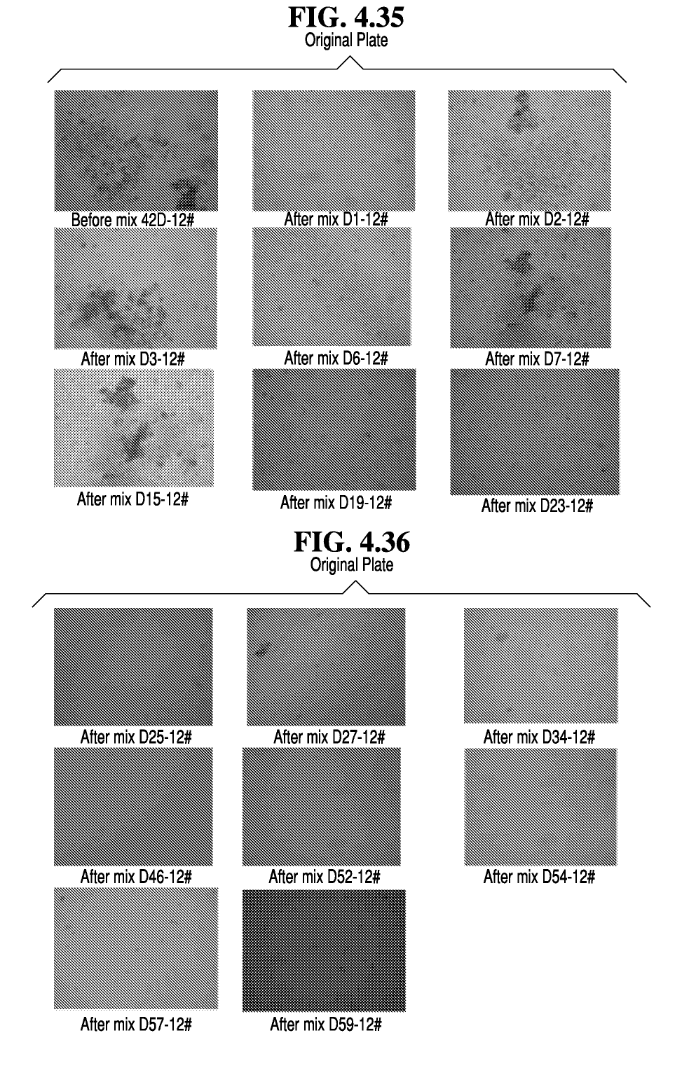

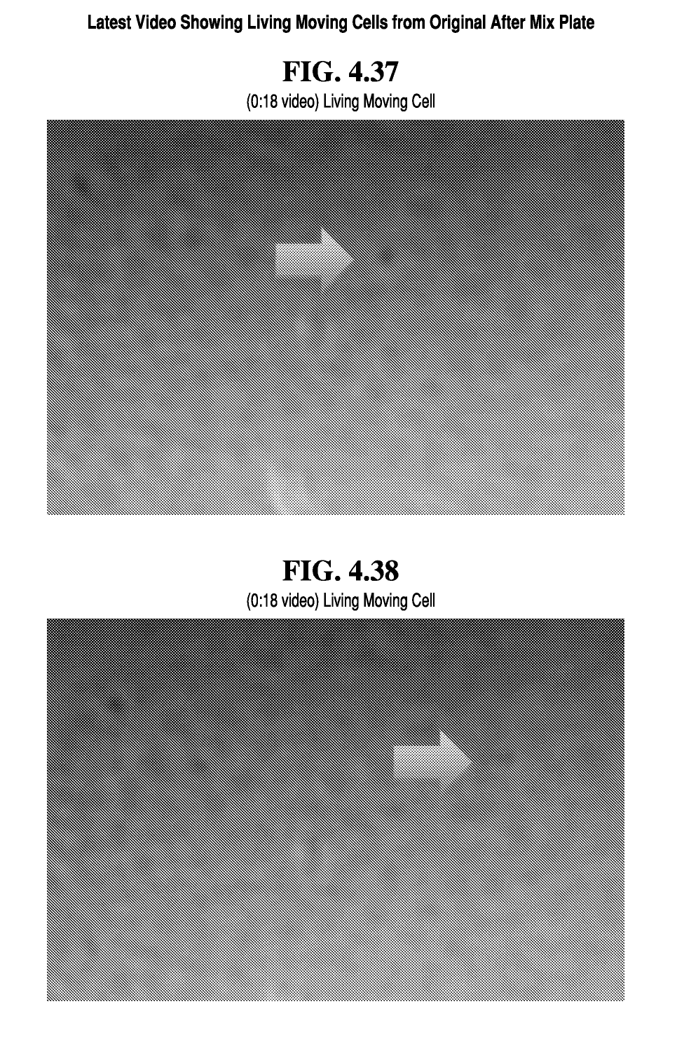

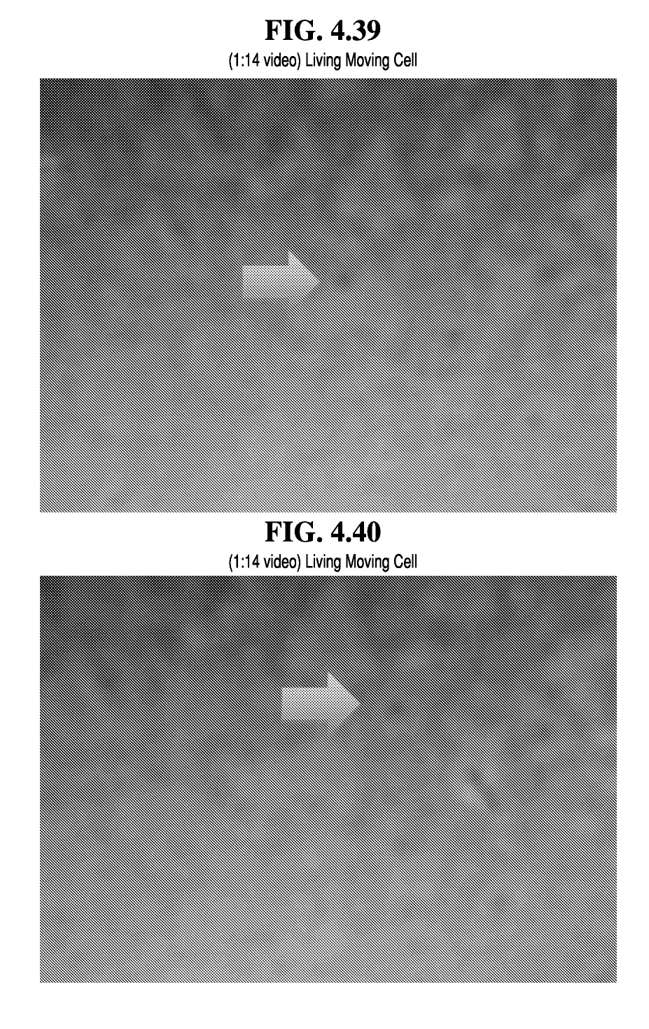

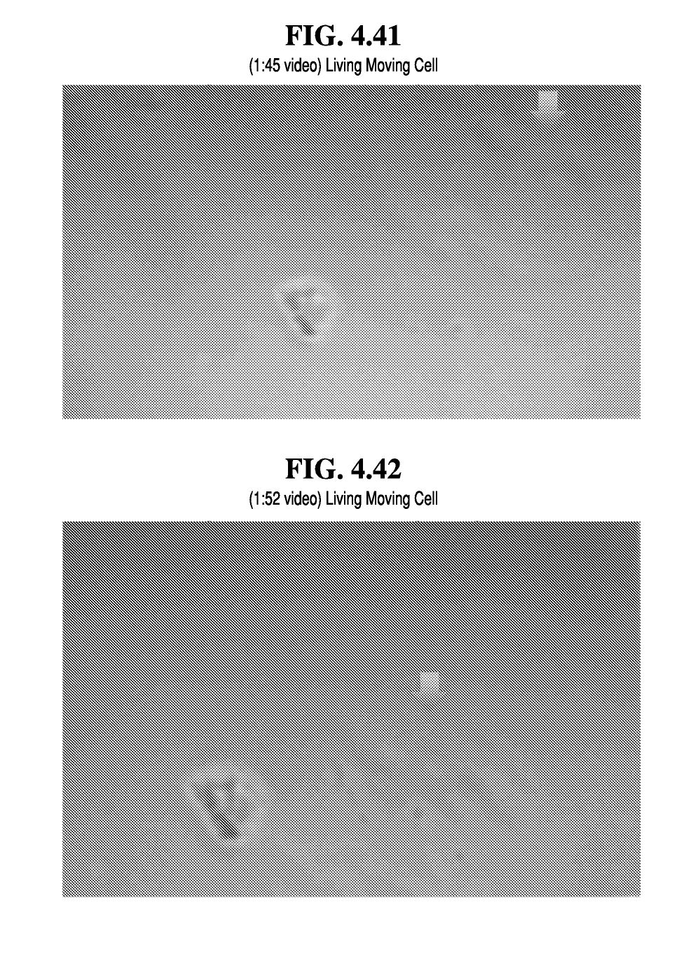

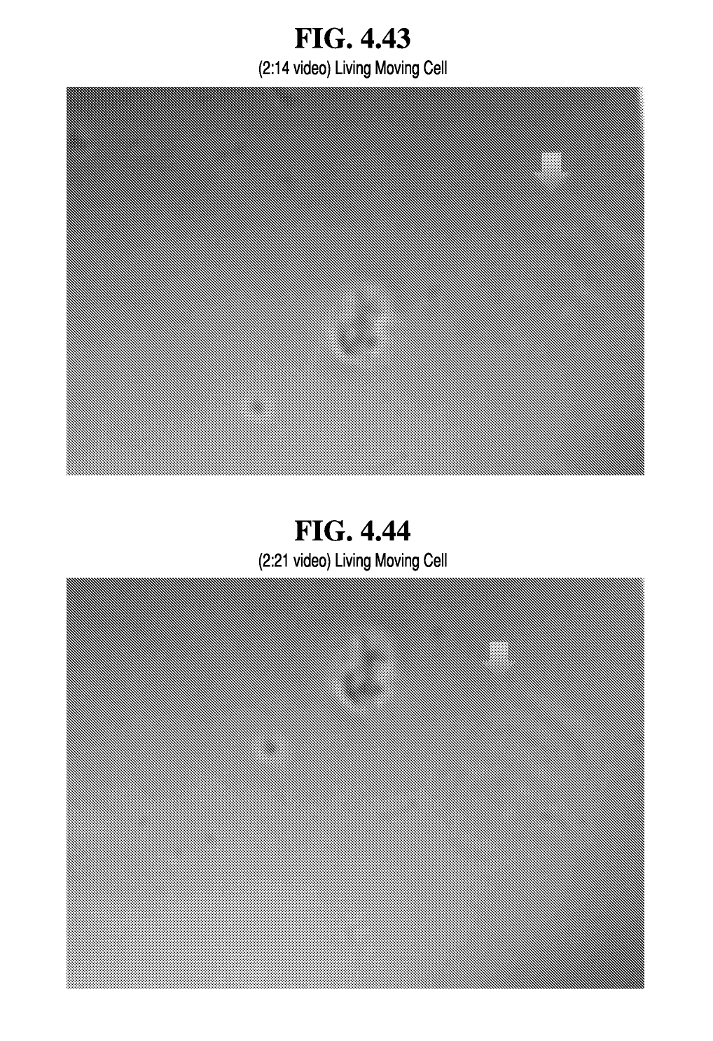

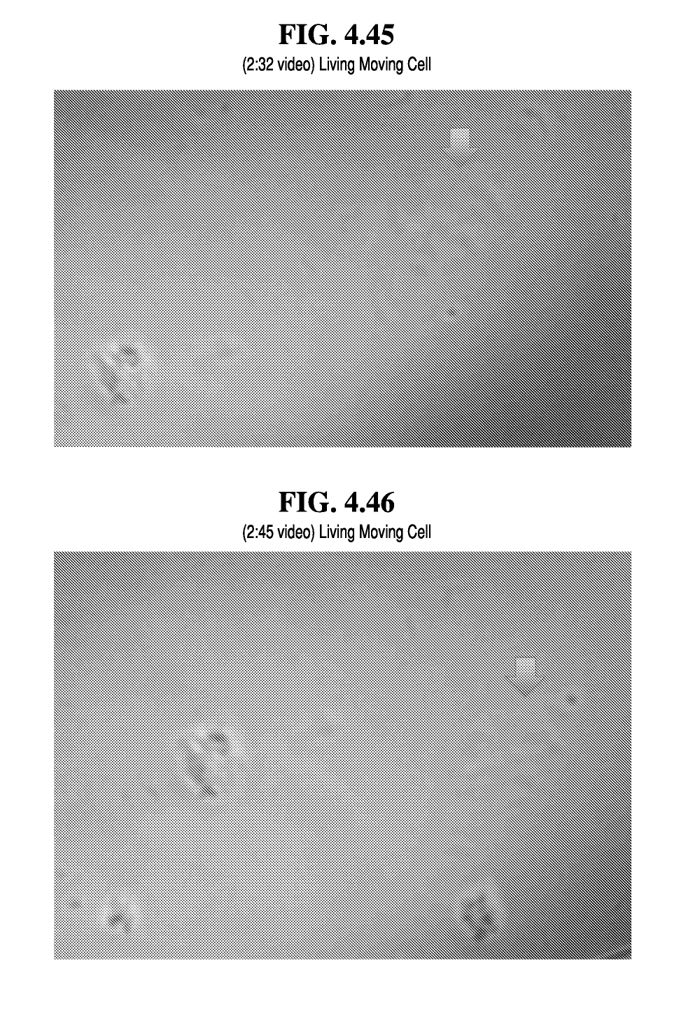

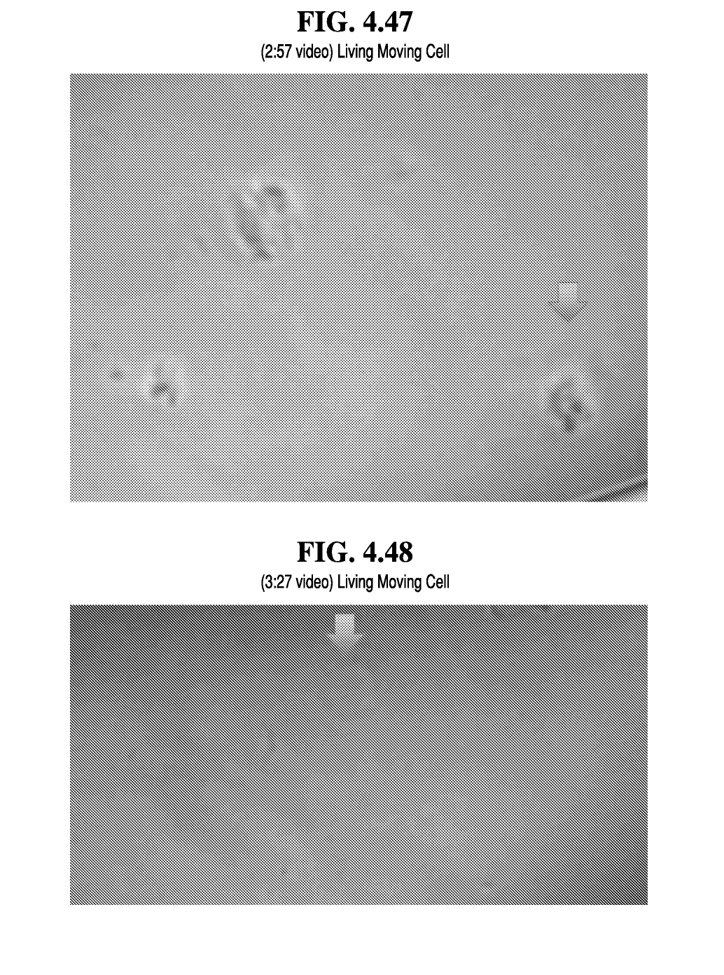

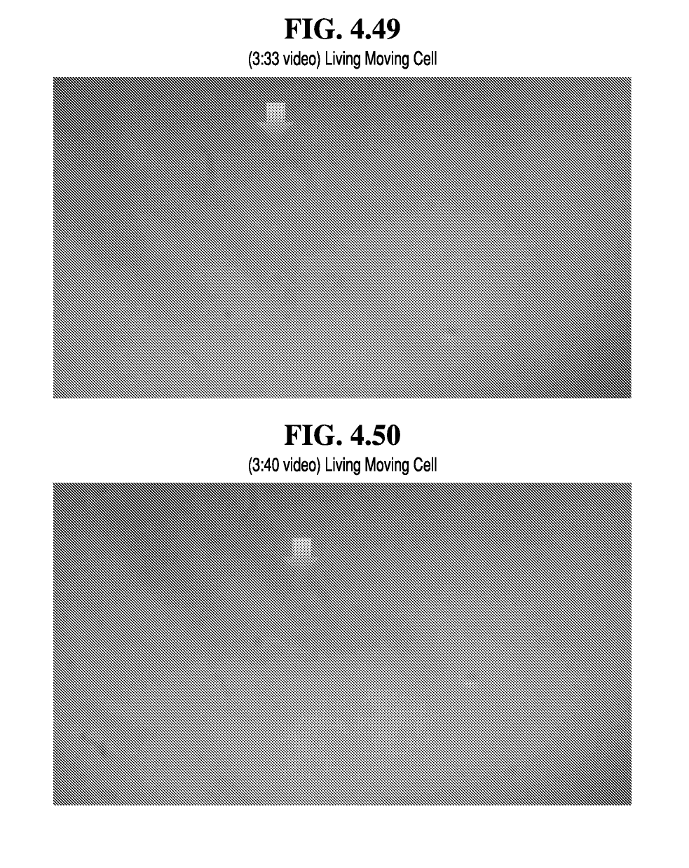

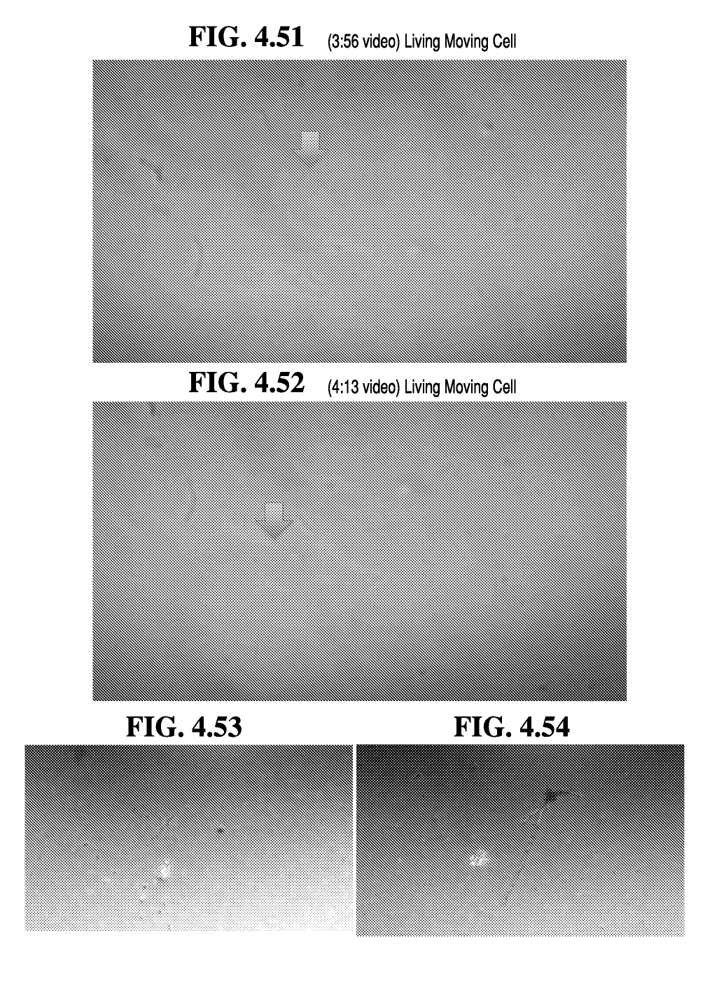

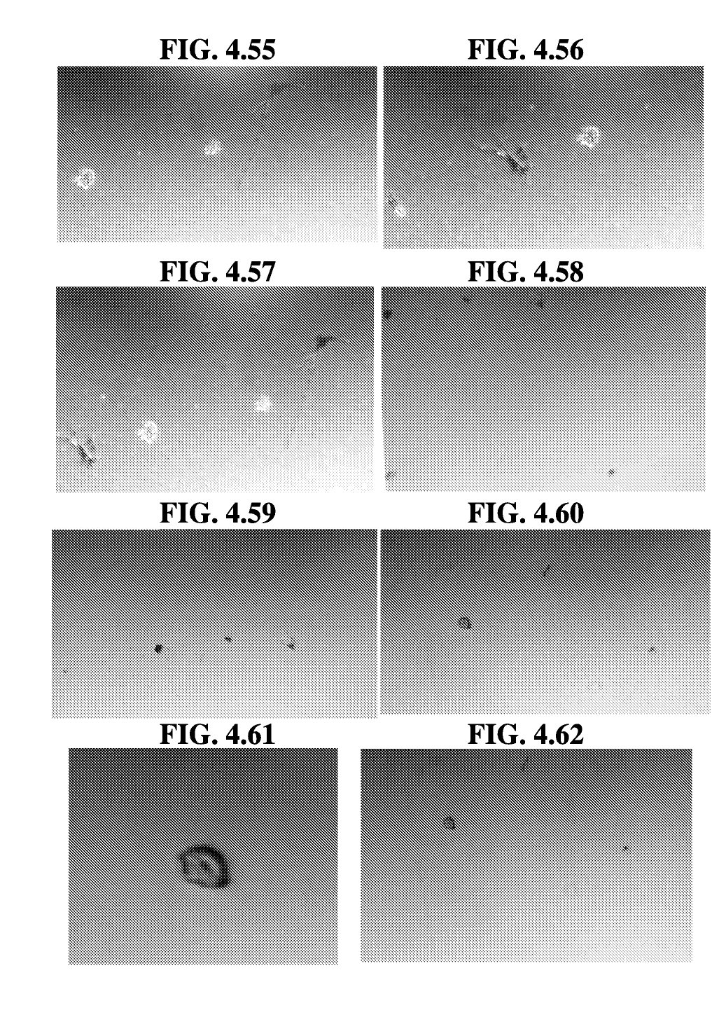

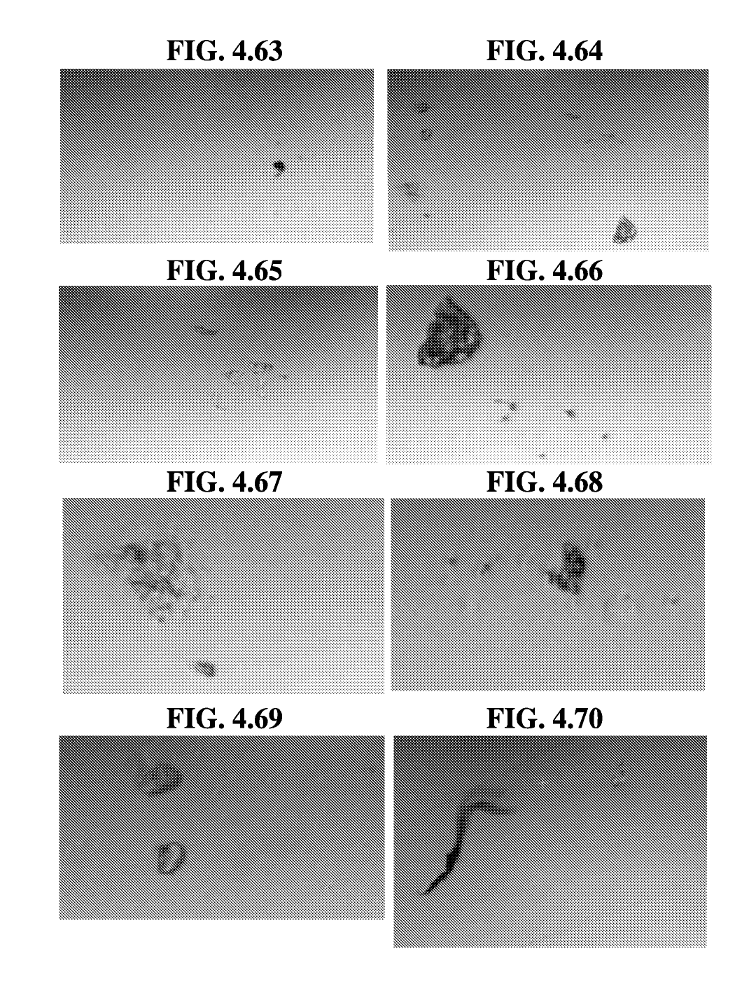

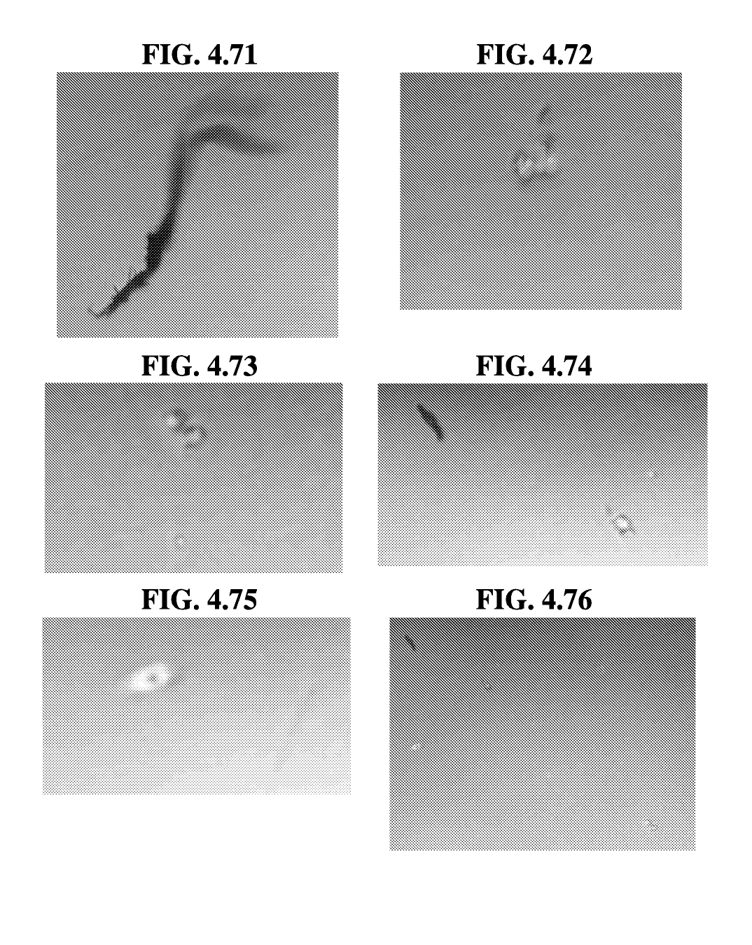

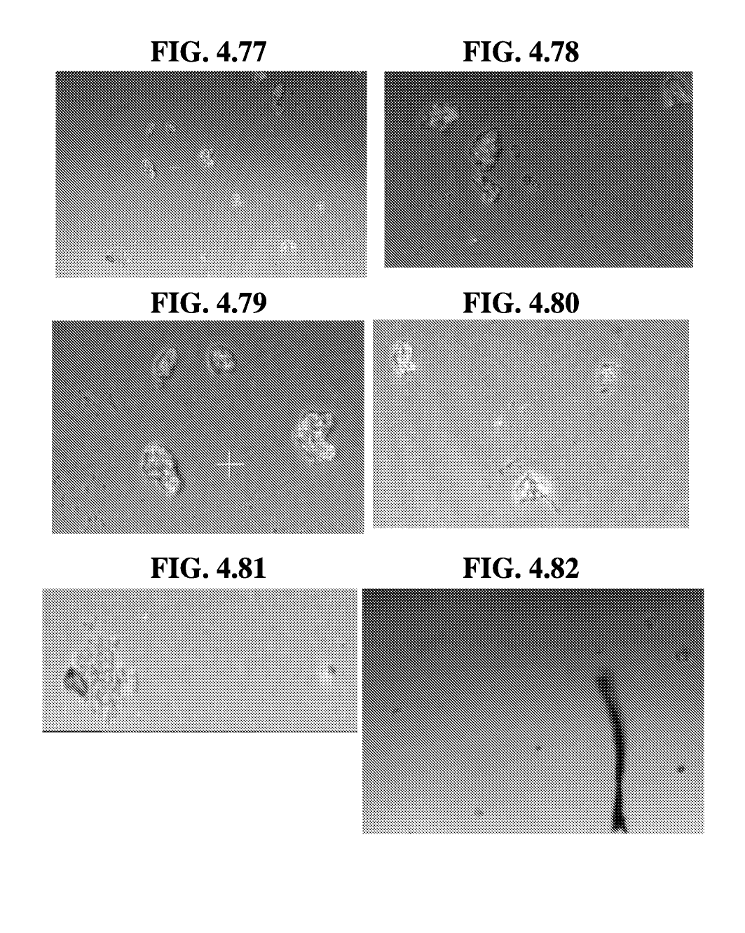

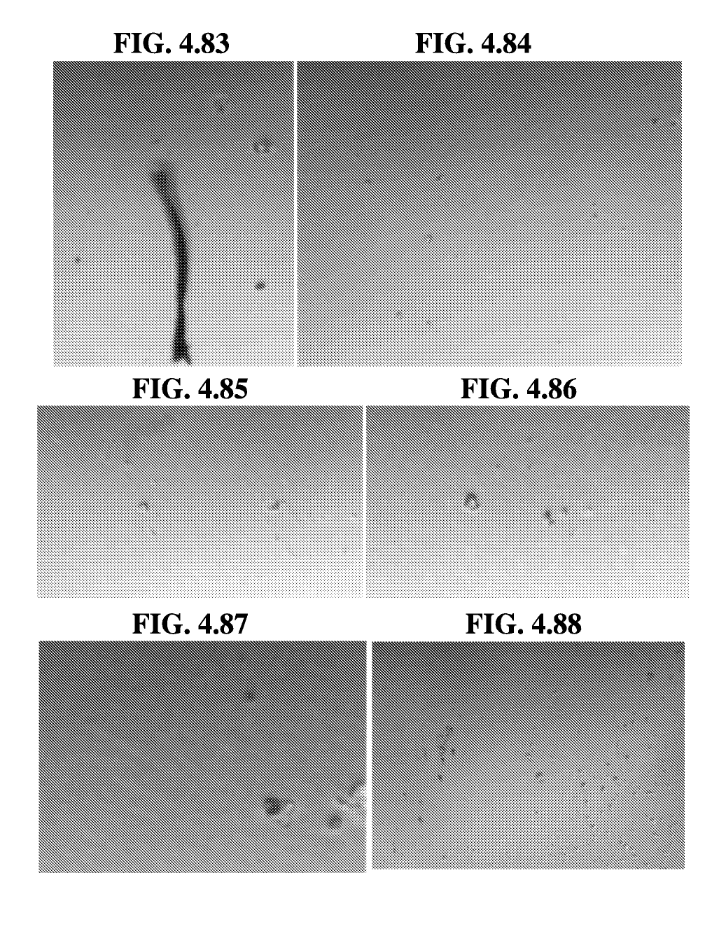

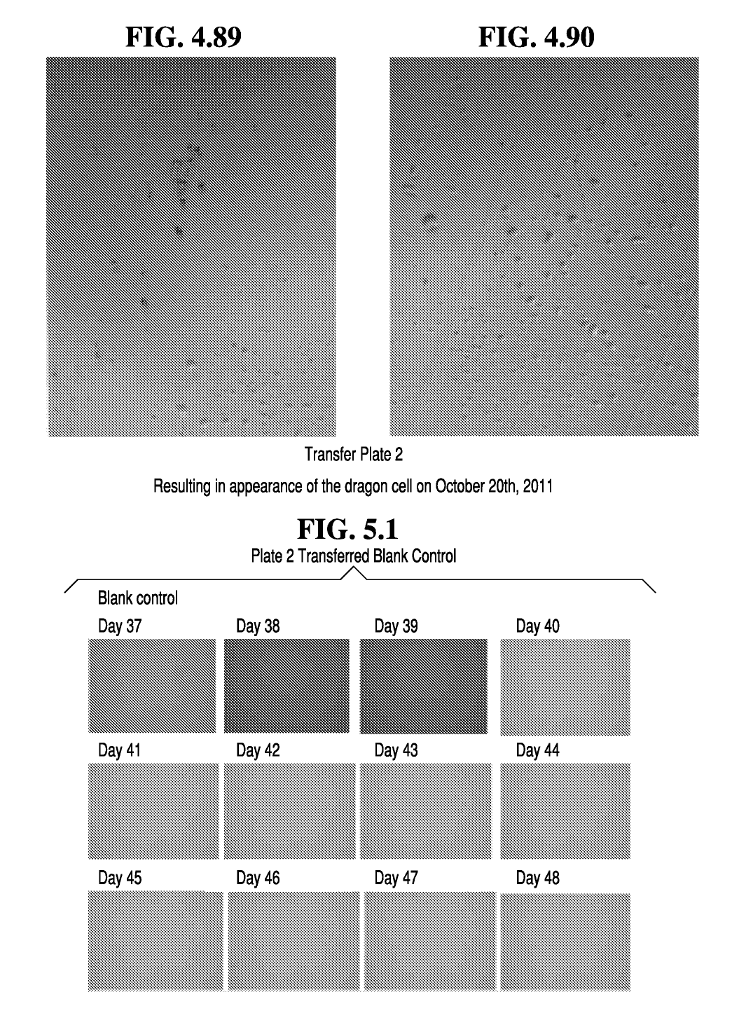

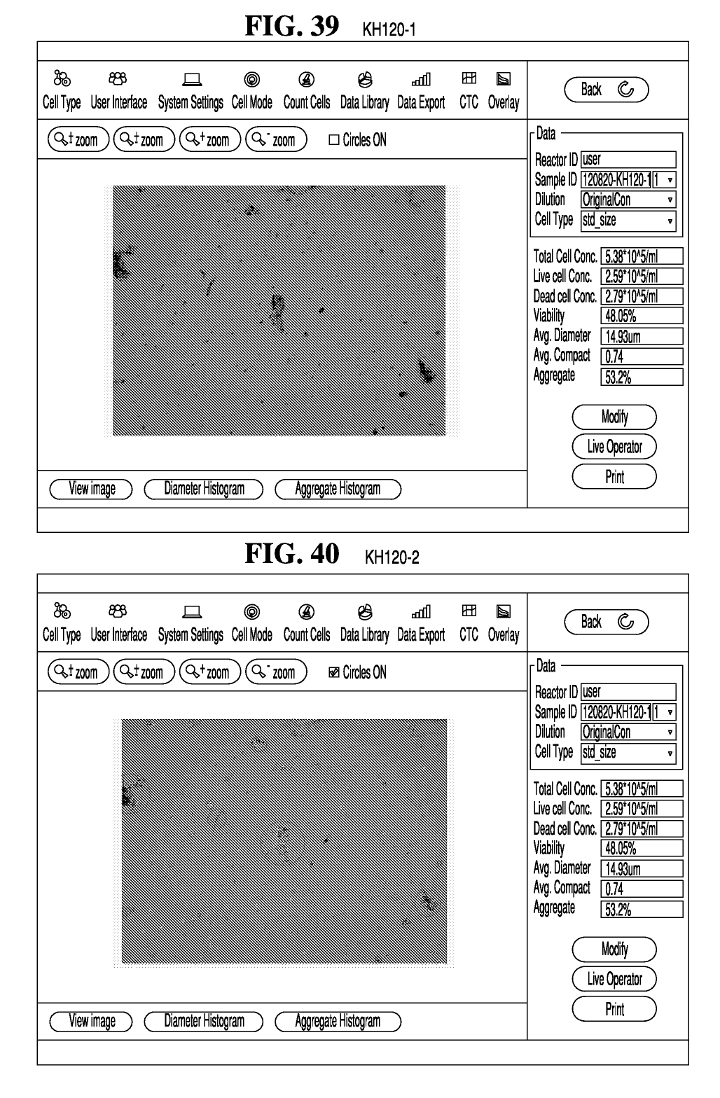

FIGS. 39 and 4—KH121 consists of 50 g of paste of Corn into 1000 mL of water for injection.

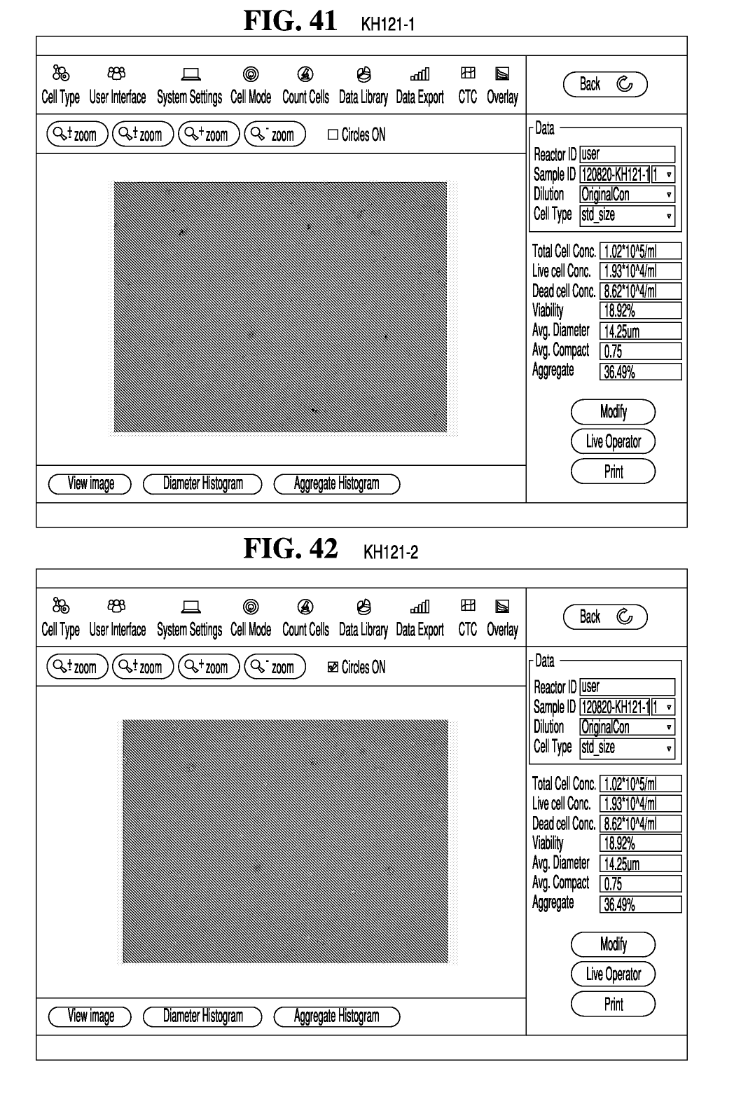

FIGS. 41 and 4—KH122 consists of 50 g of paste of Sweet Potato into 100 mL of water for injection.

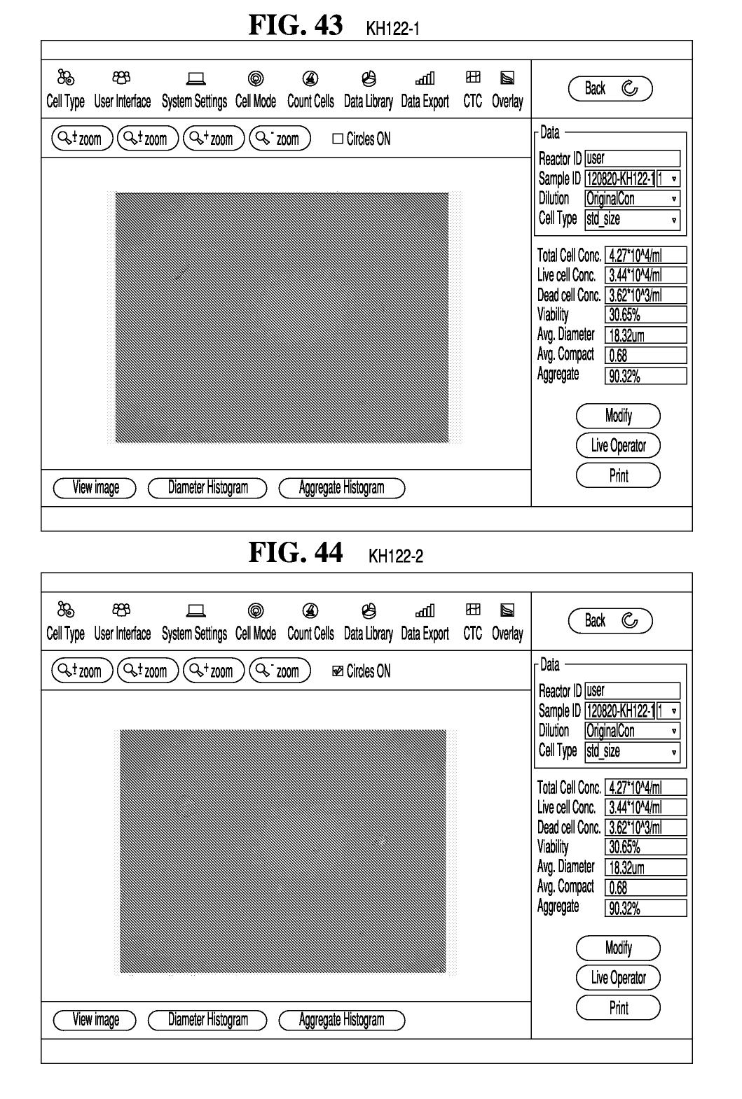

FIGS. 43 and 4—KH123 consists of 2 g of paste of Cucumber into 800 mL of water for injection.

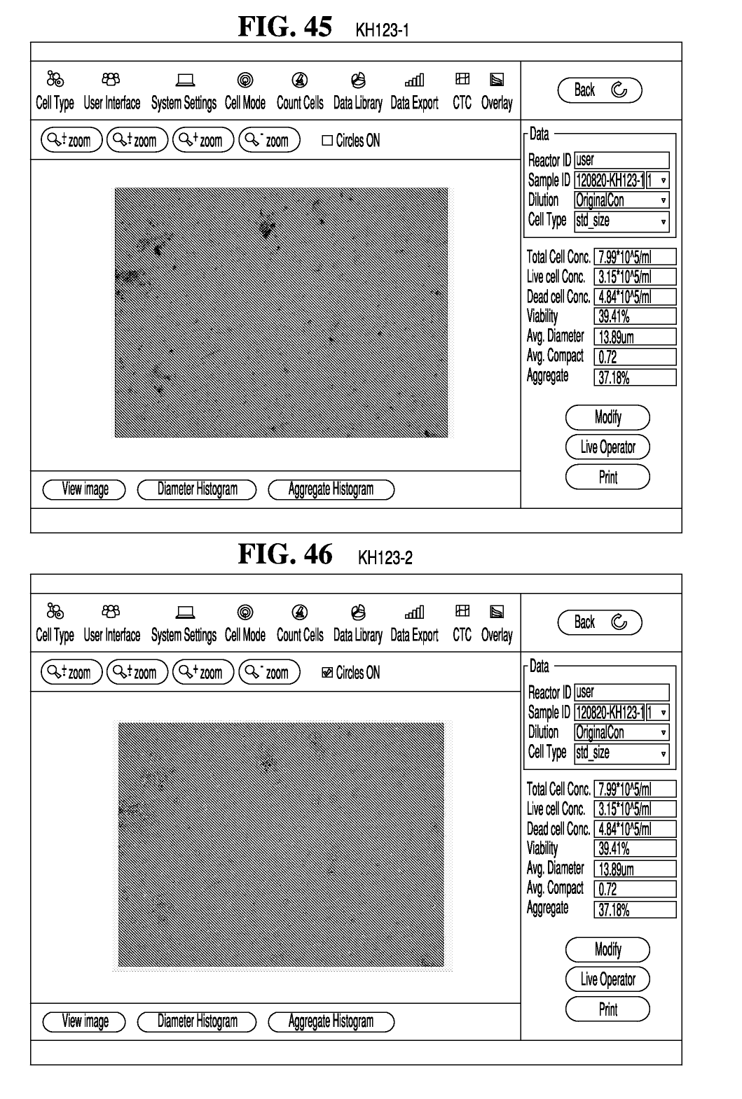

FIGS. 45 and 4—KH124 consists of 44 g of paste of Tomato into 800 mL of water for injection.

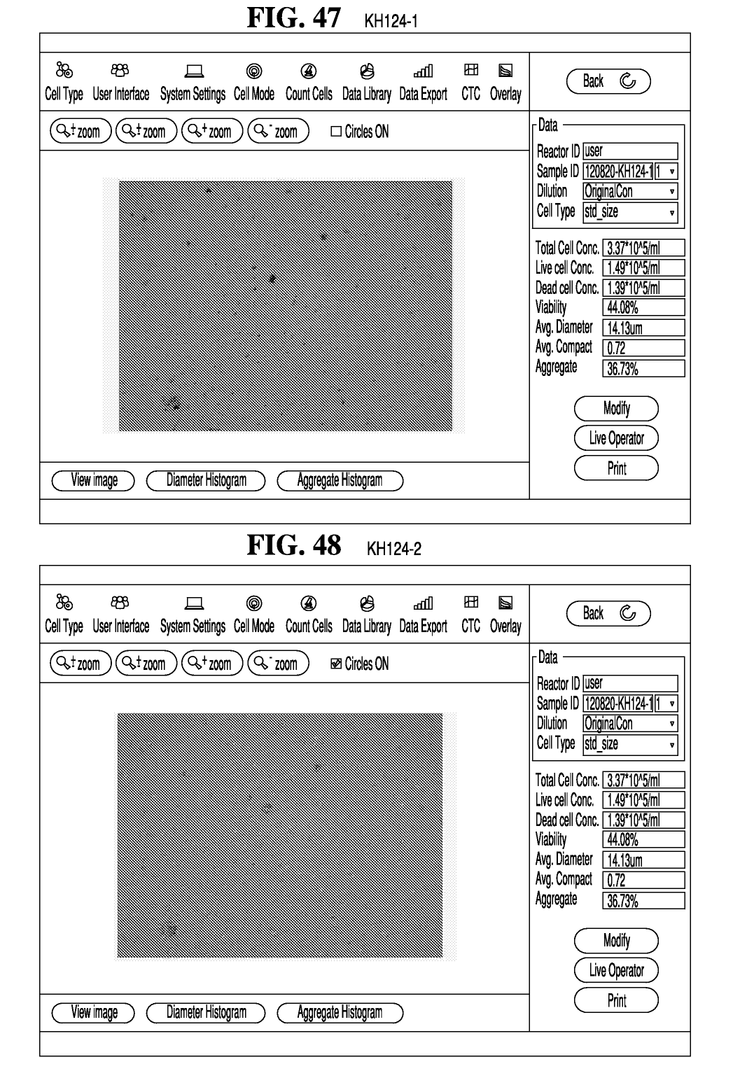

FIGS. 47 and 4—KH125 consists of 20 g of paste of Dragon Fruit into 400 mL of water for injection.

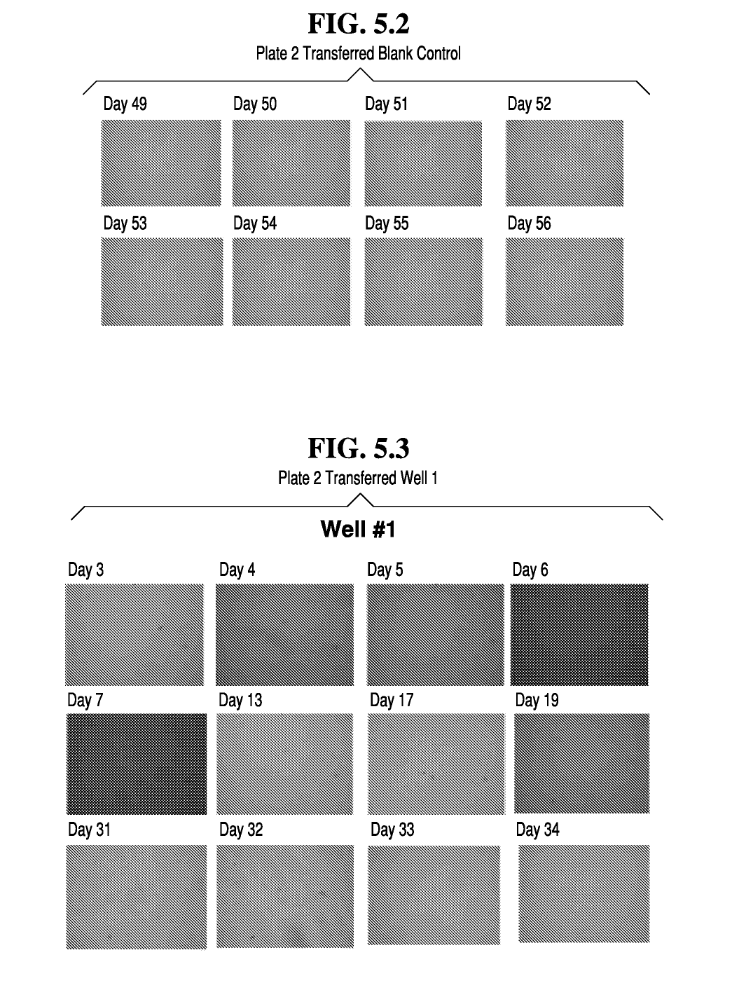

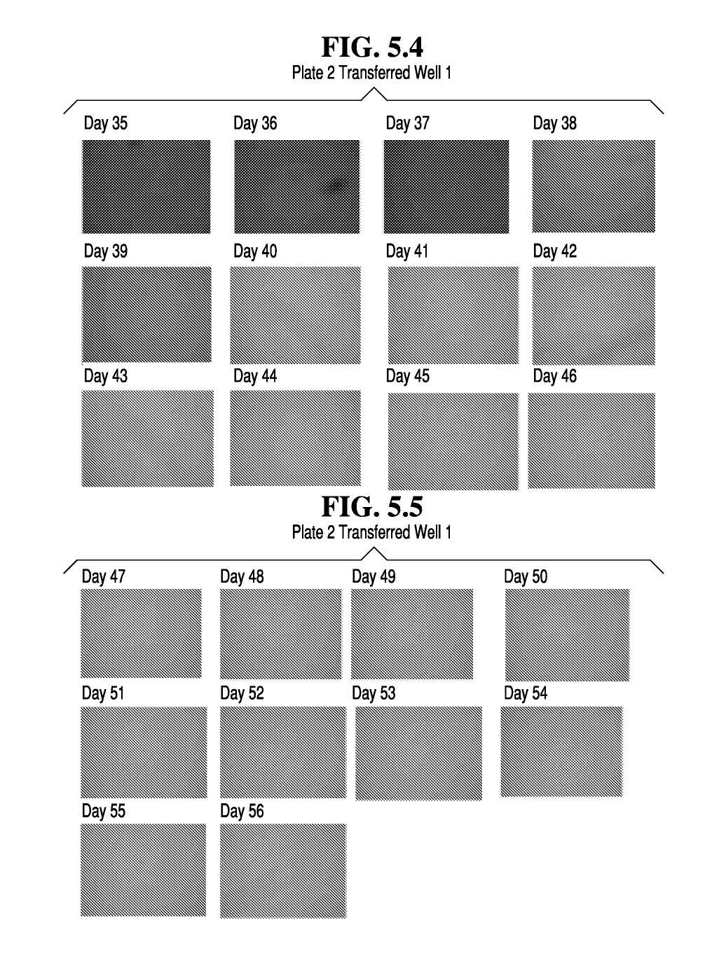

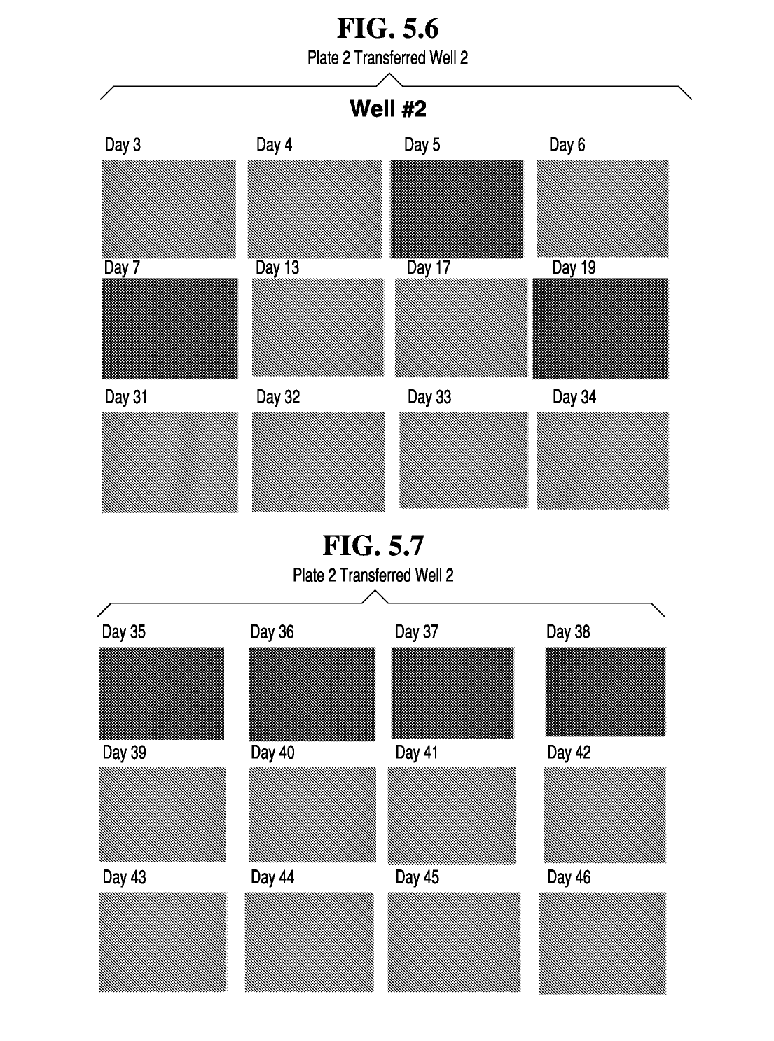

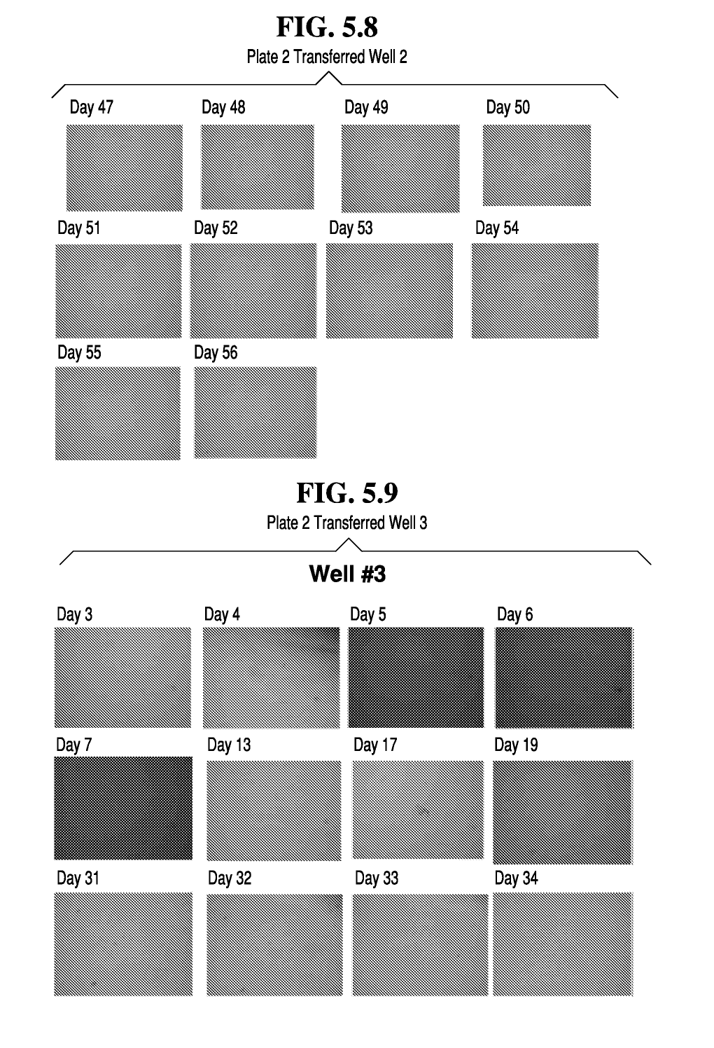

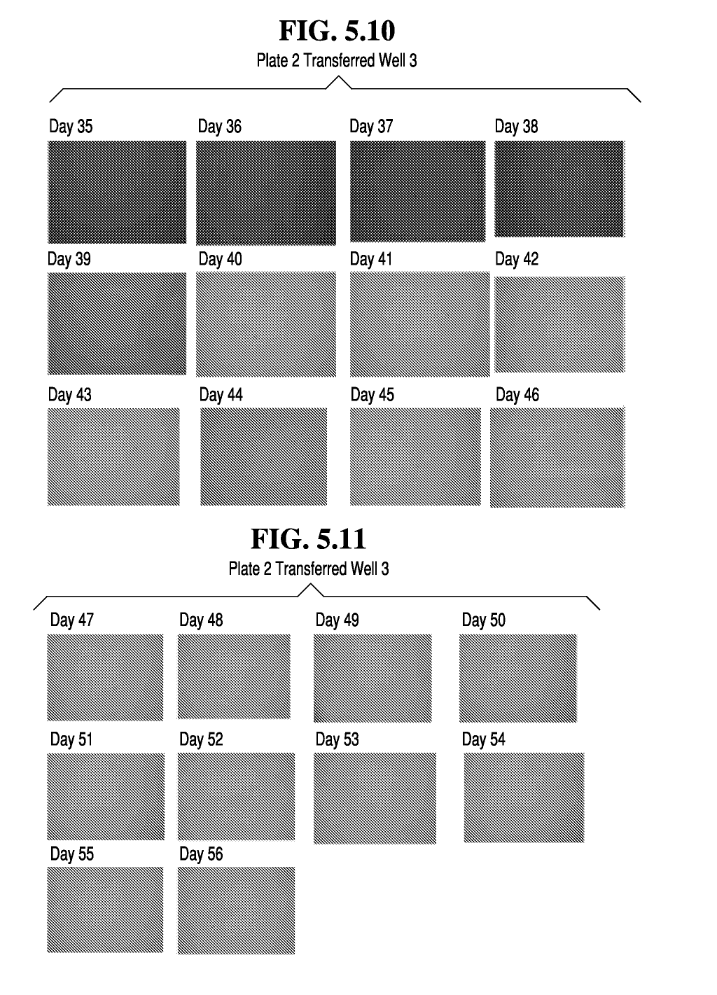

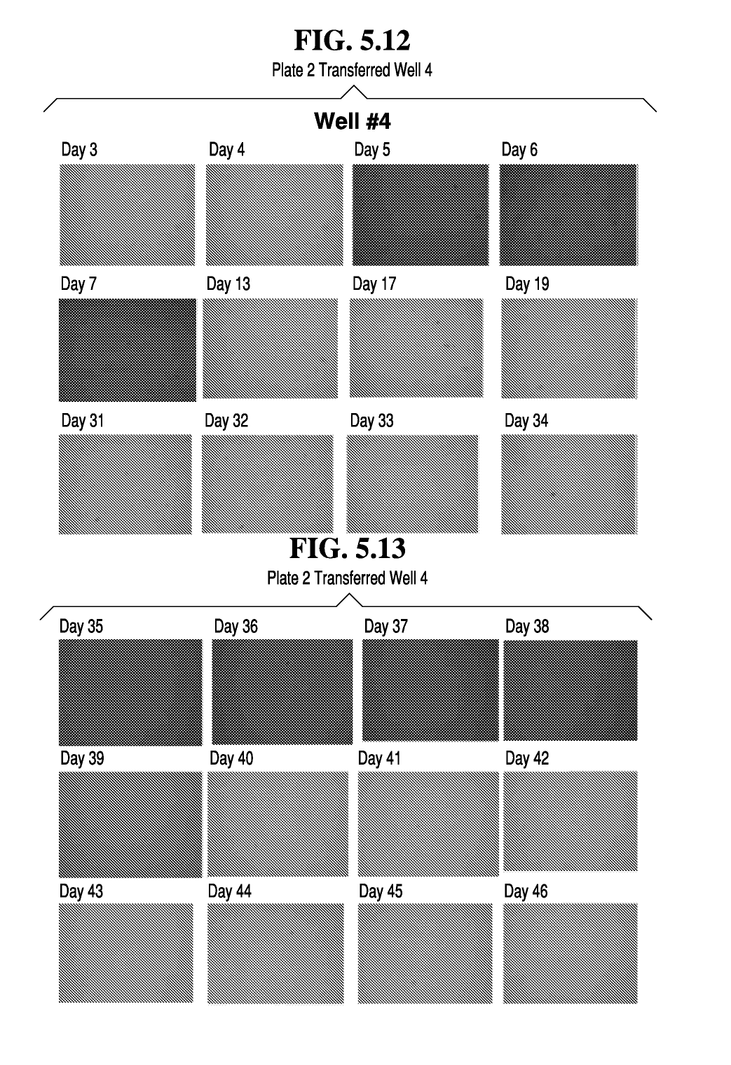

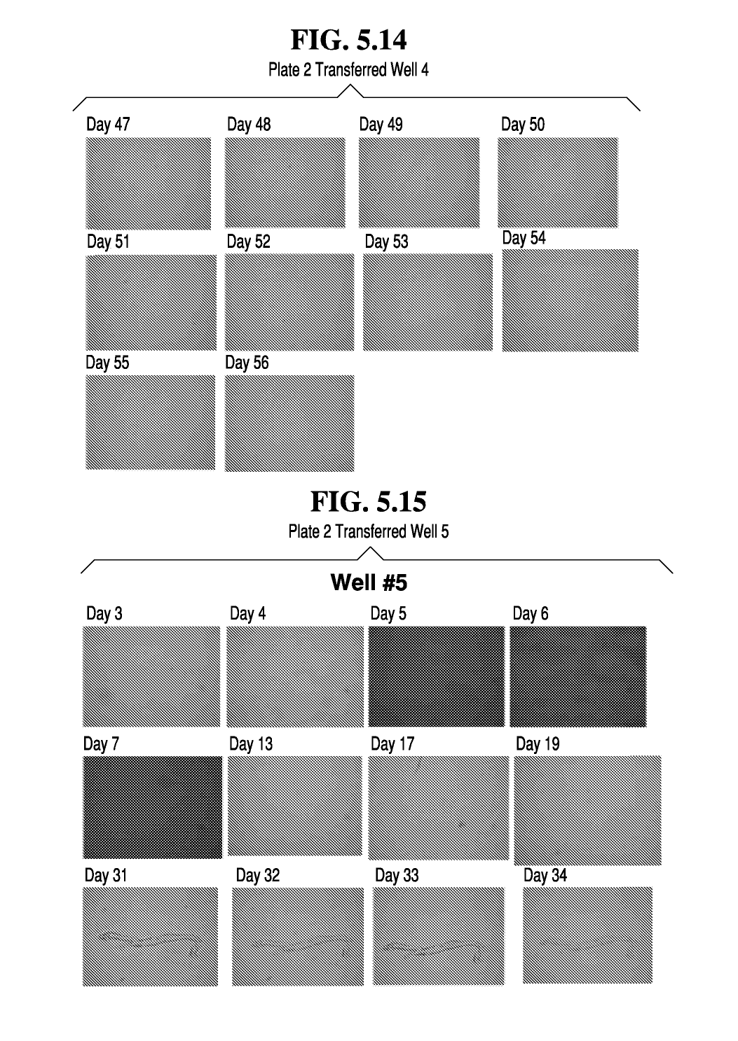

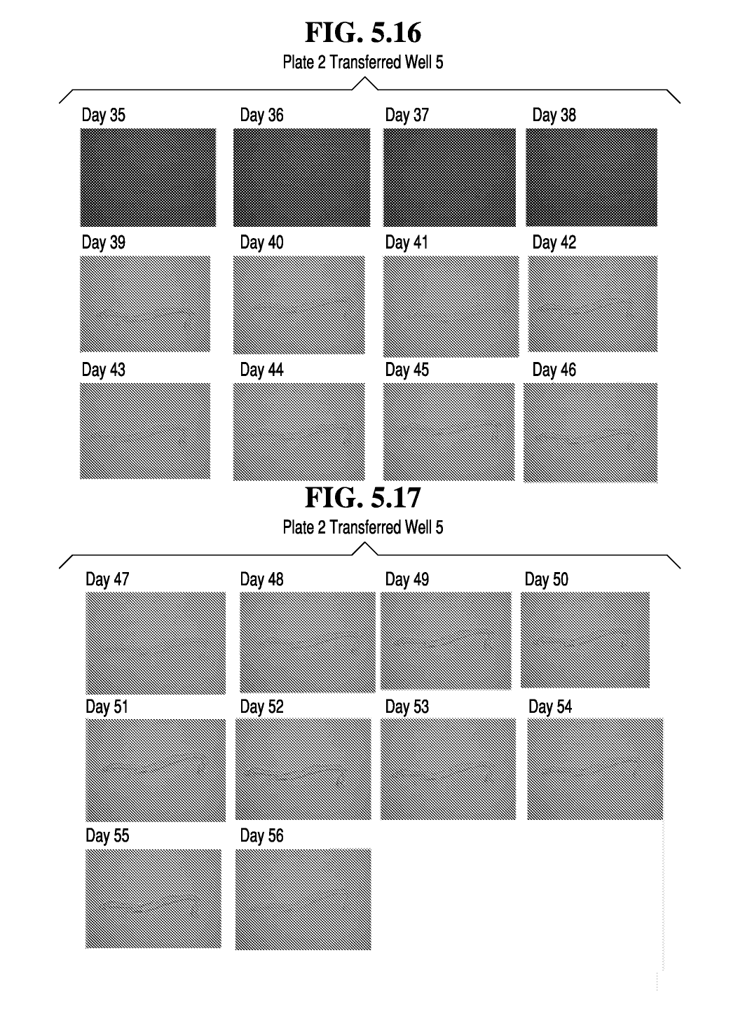

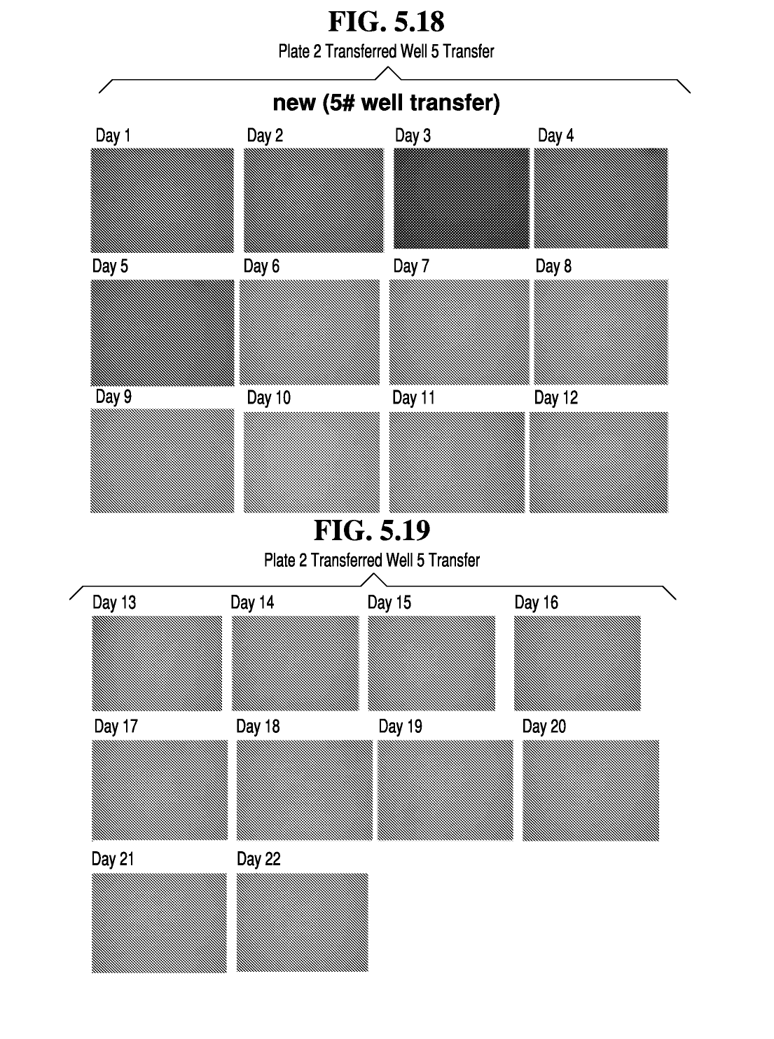

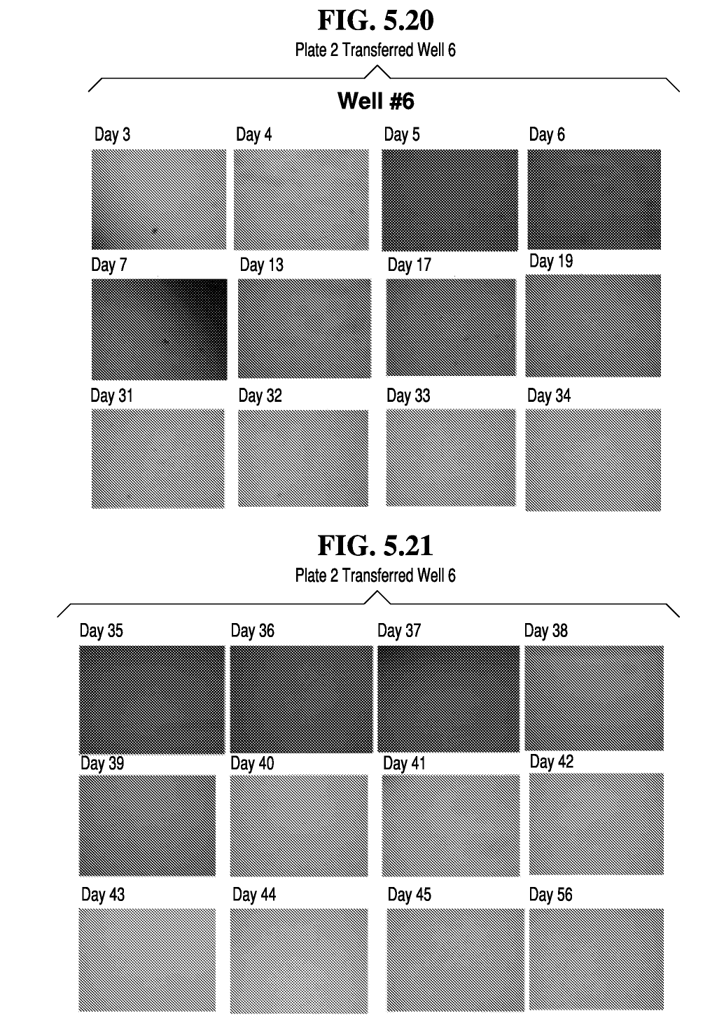

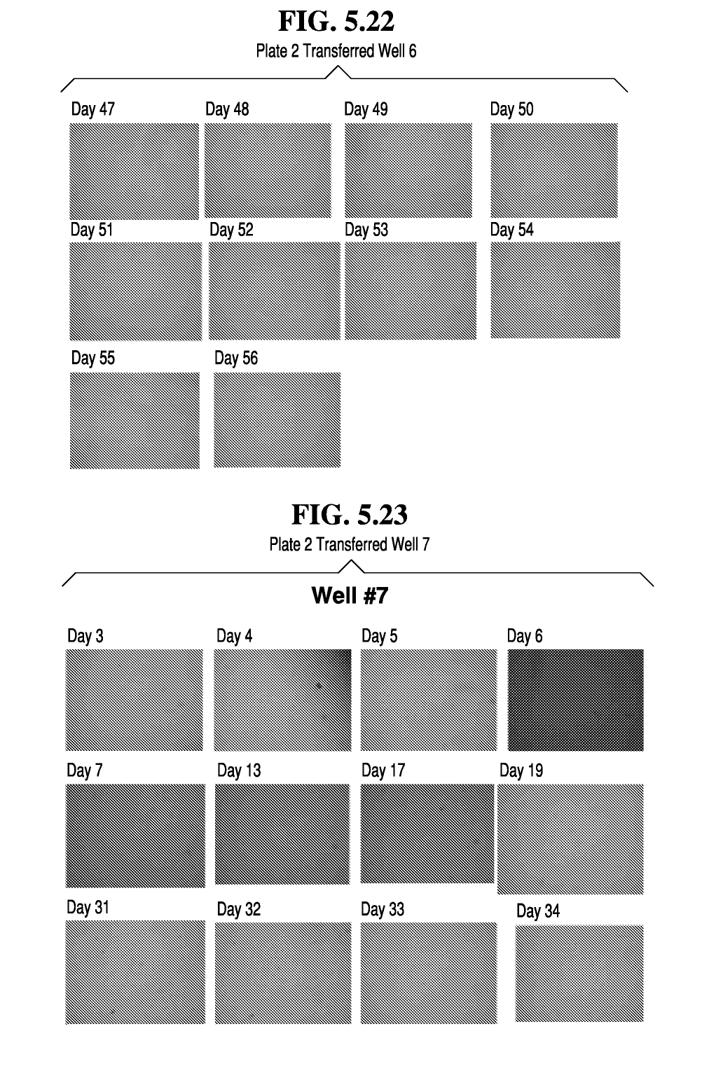

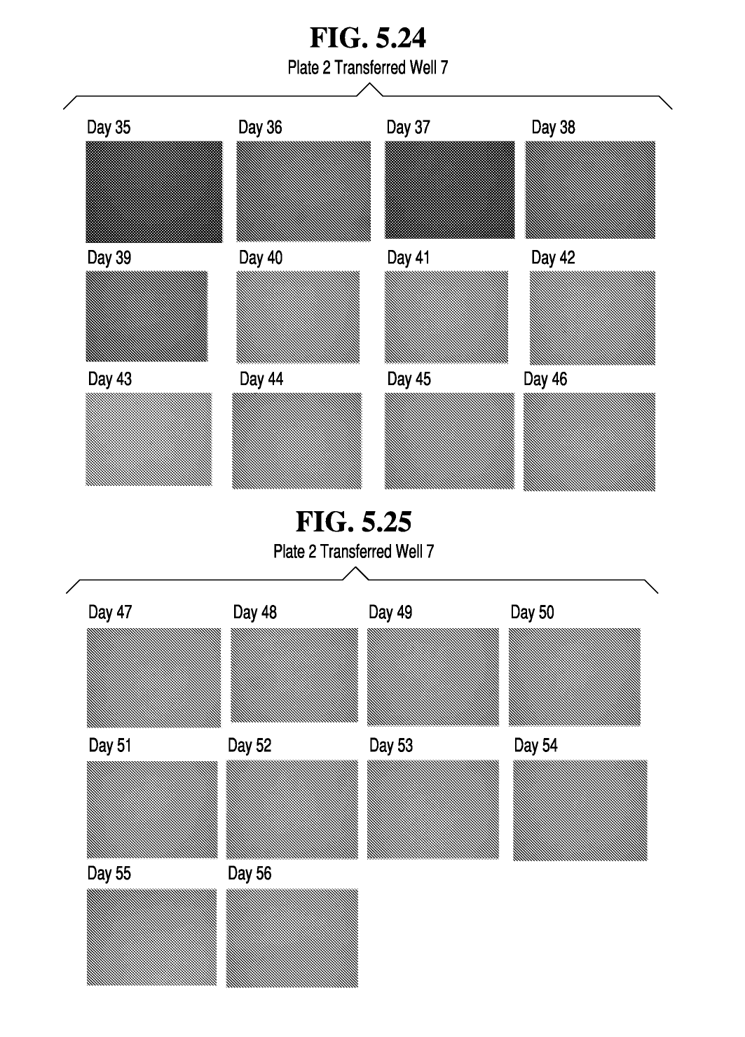

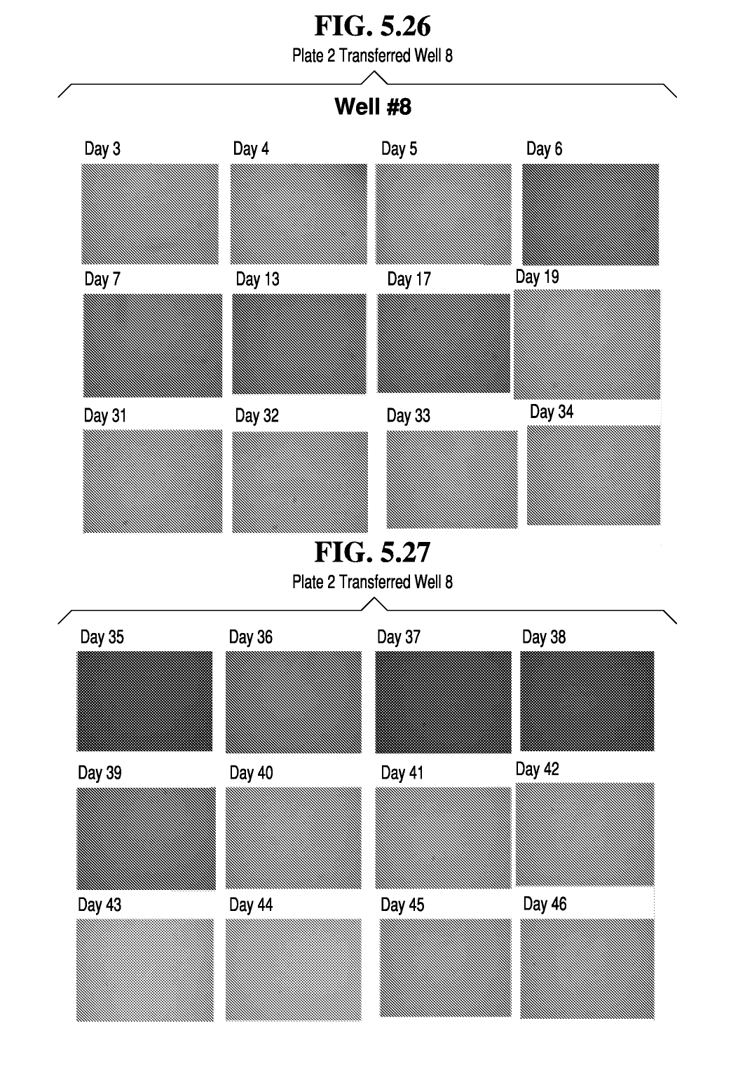

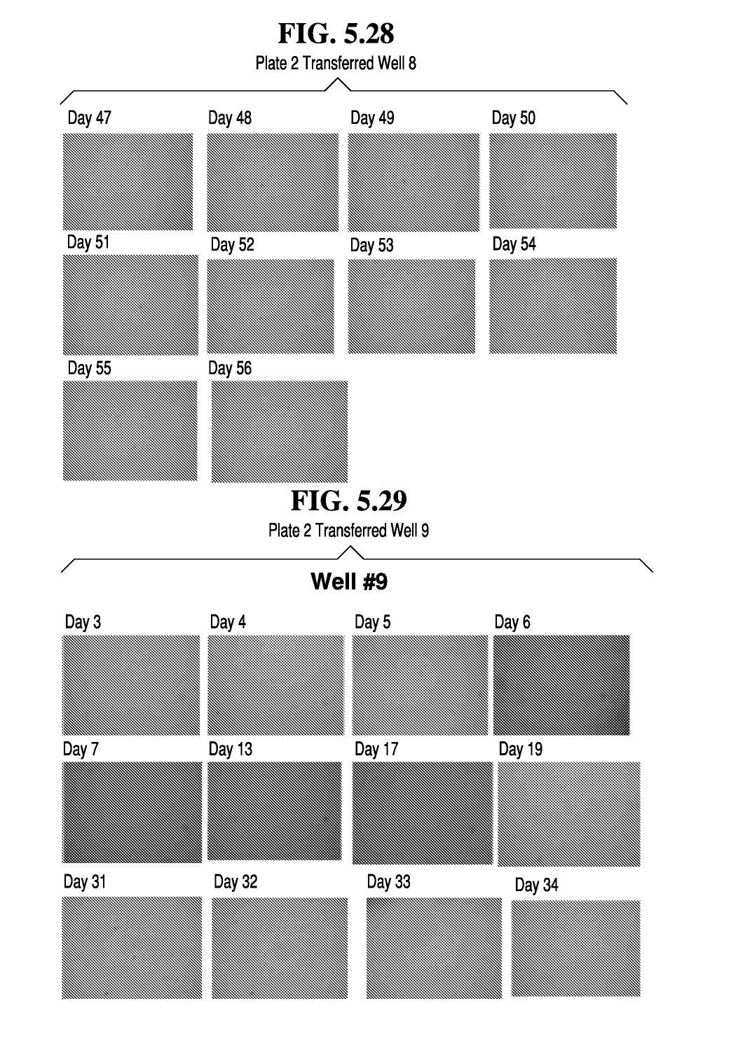

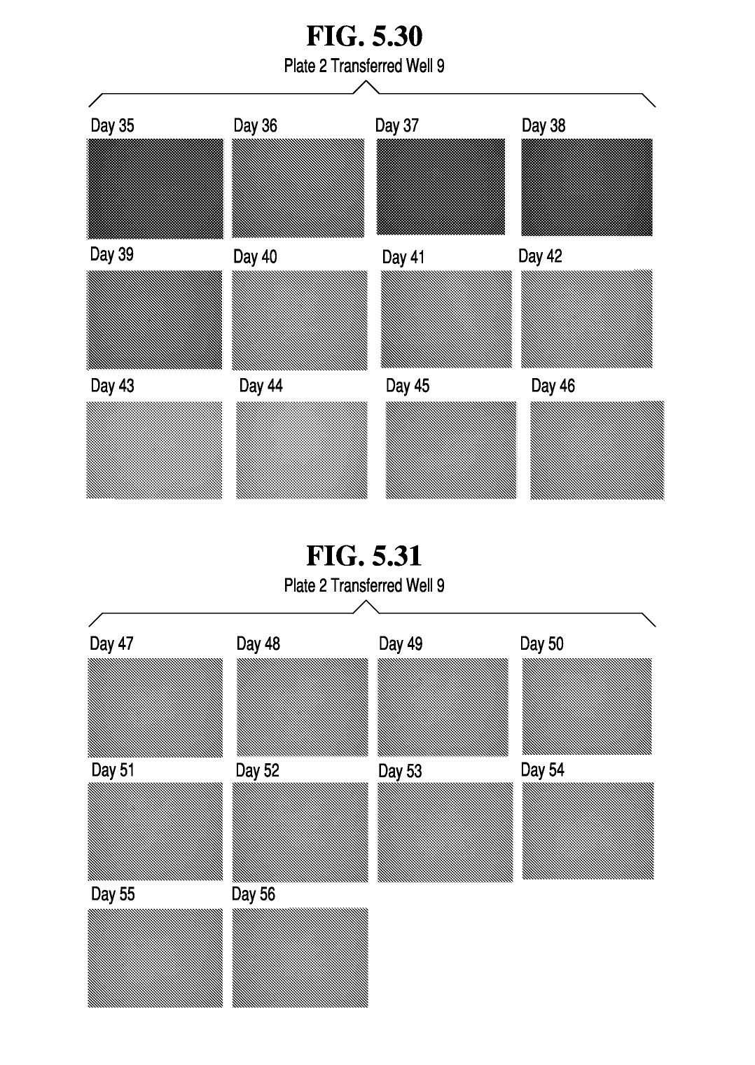

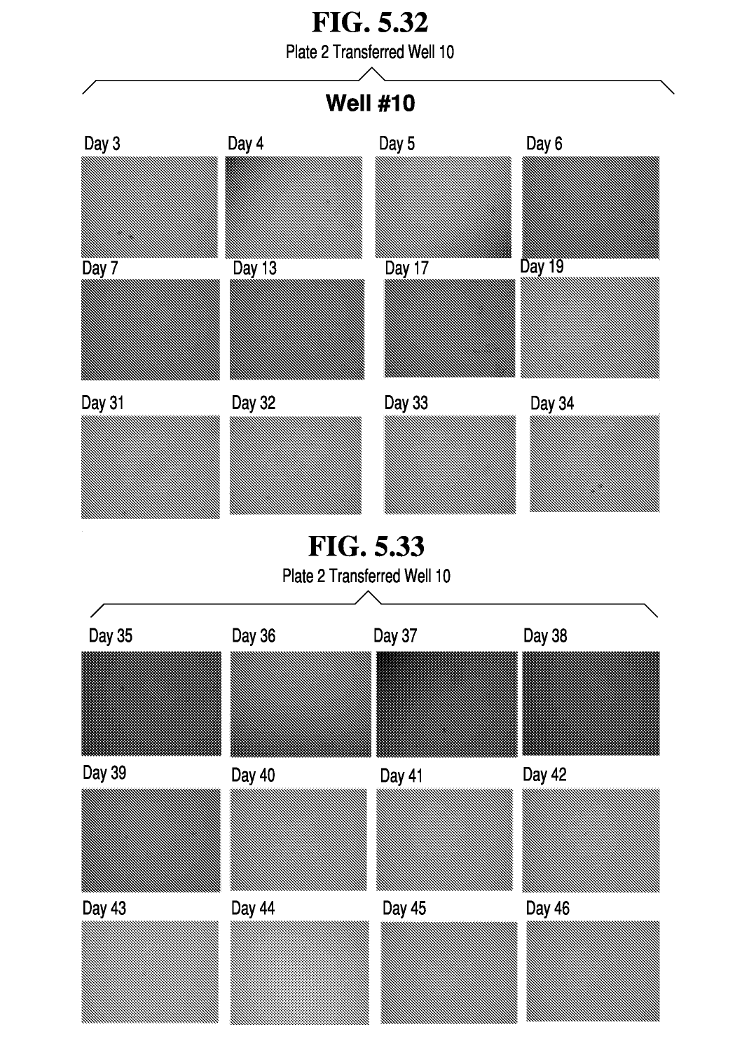

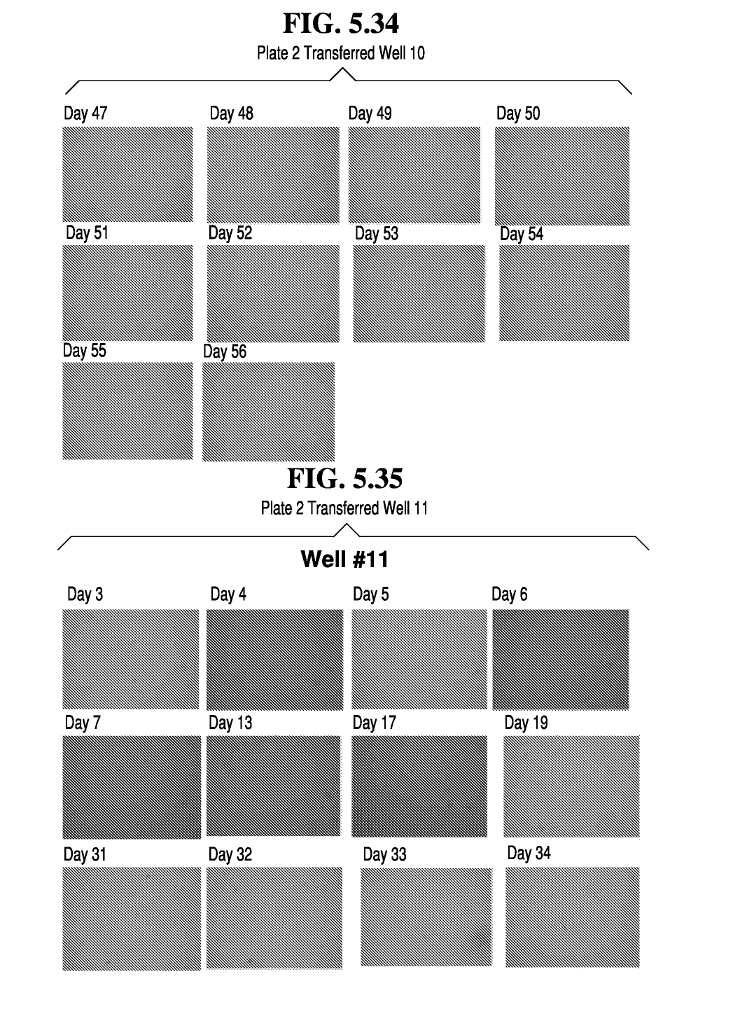

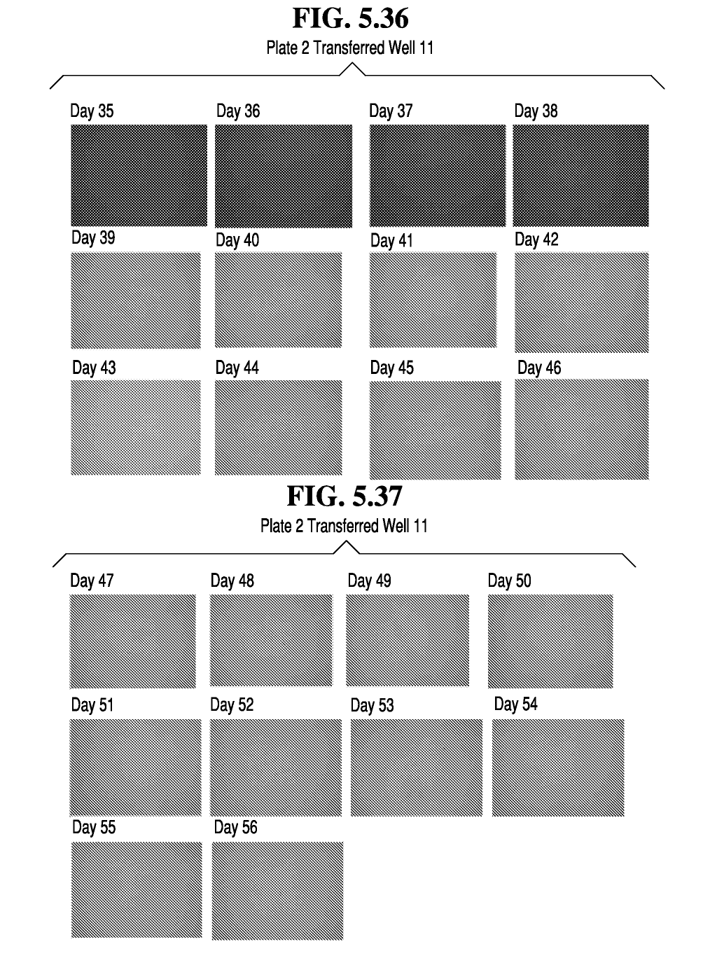

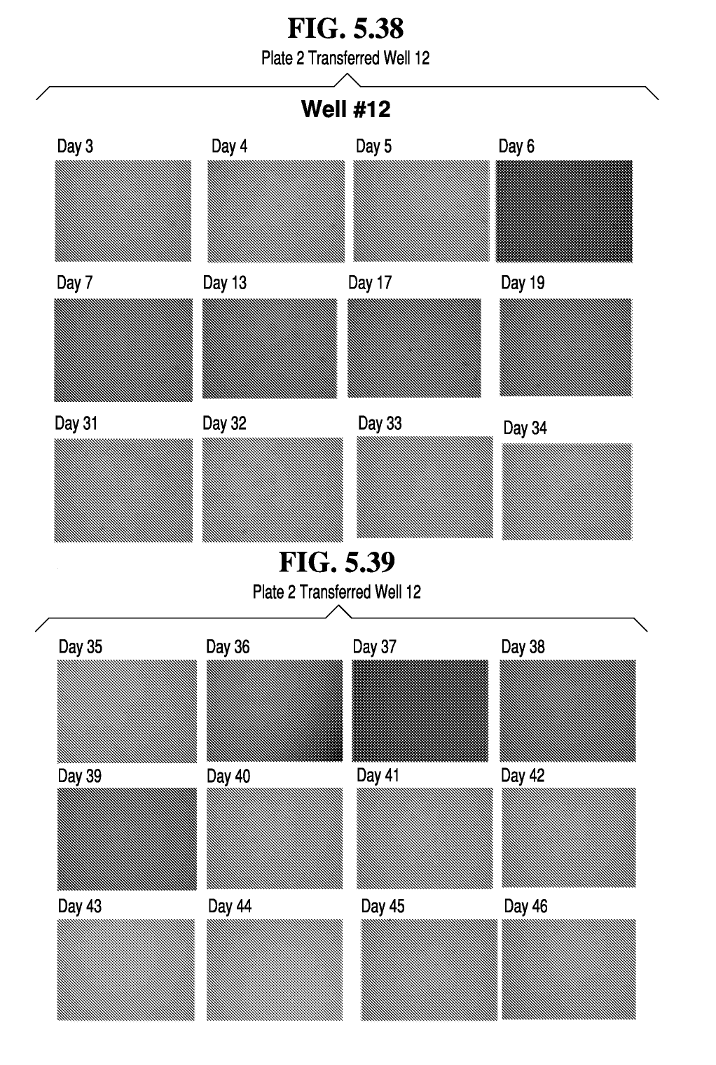

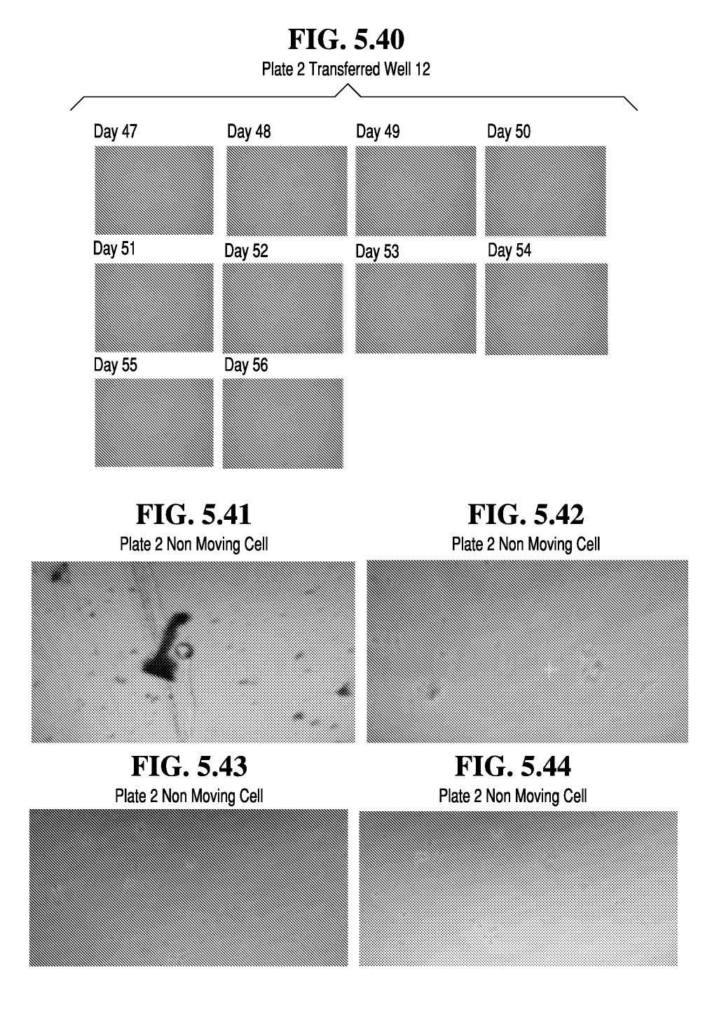

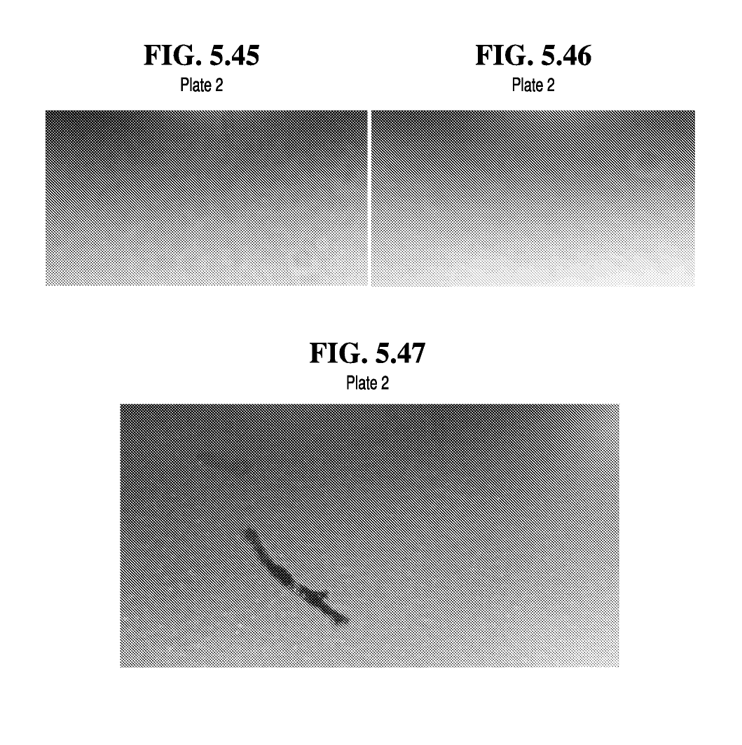

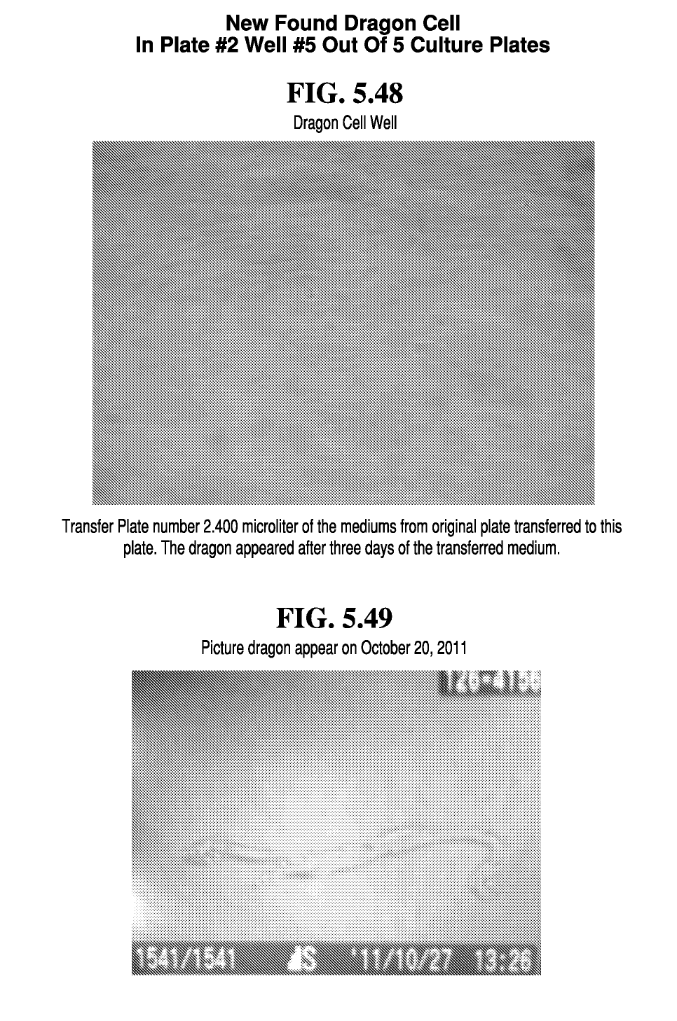

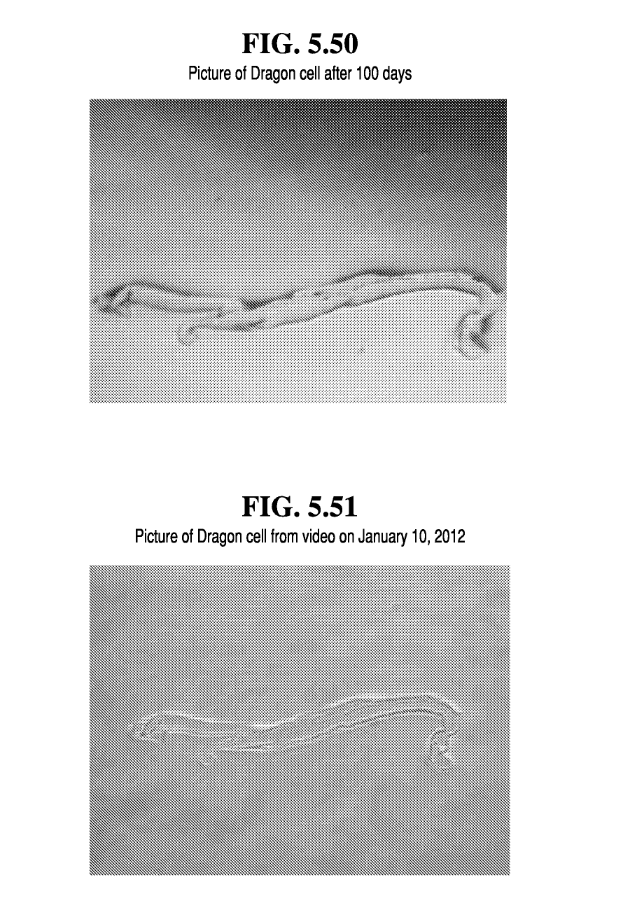

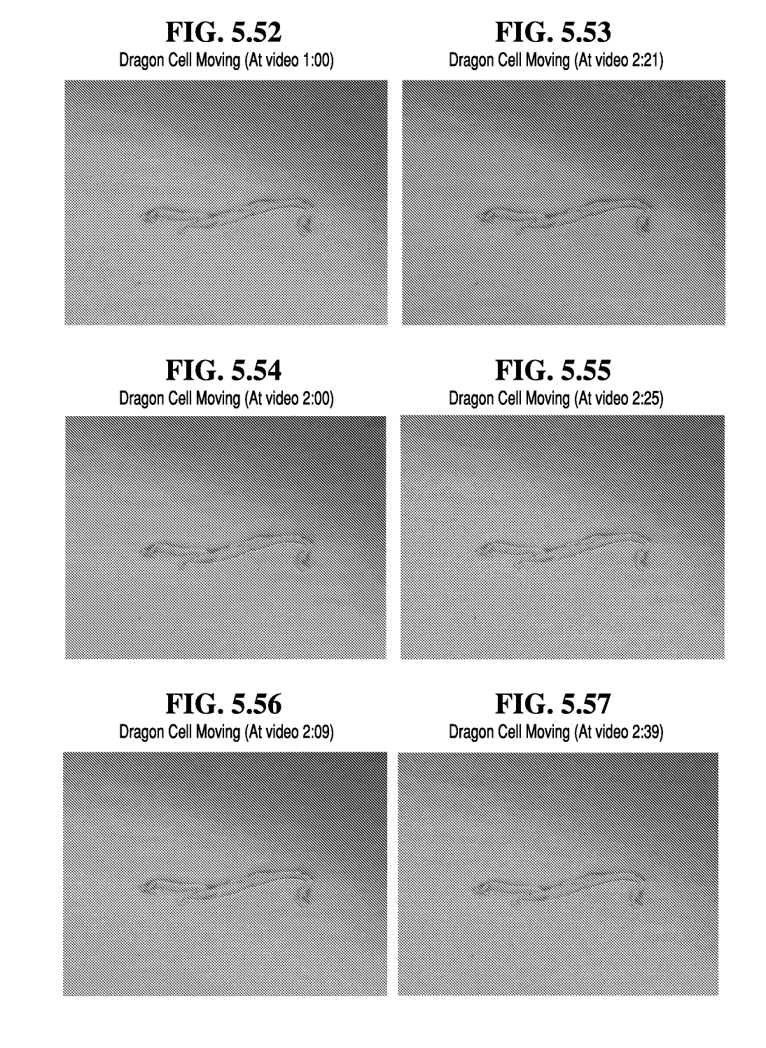

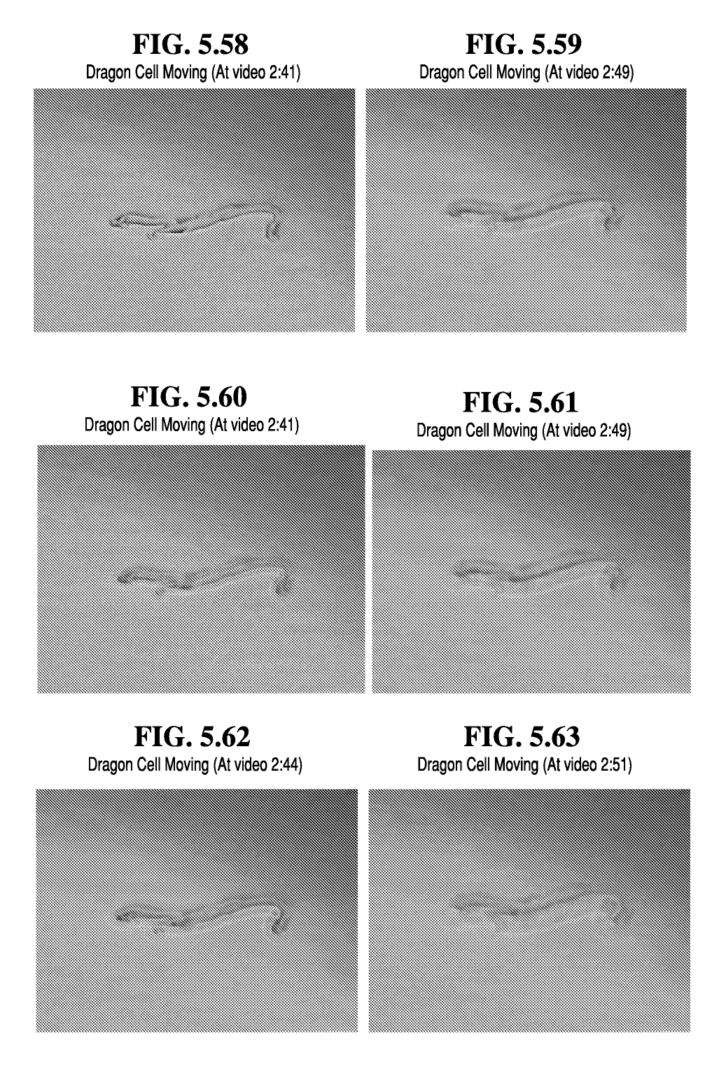

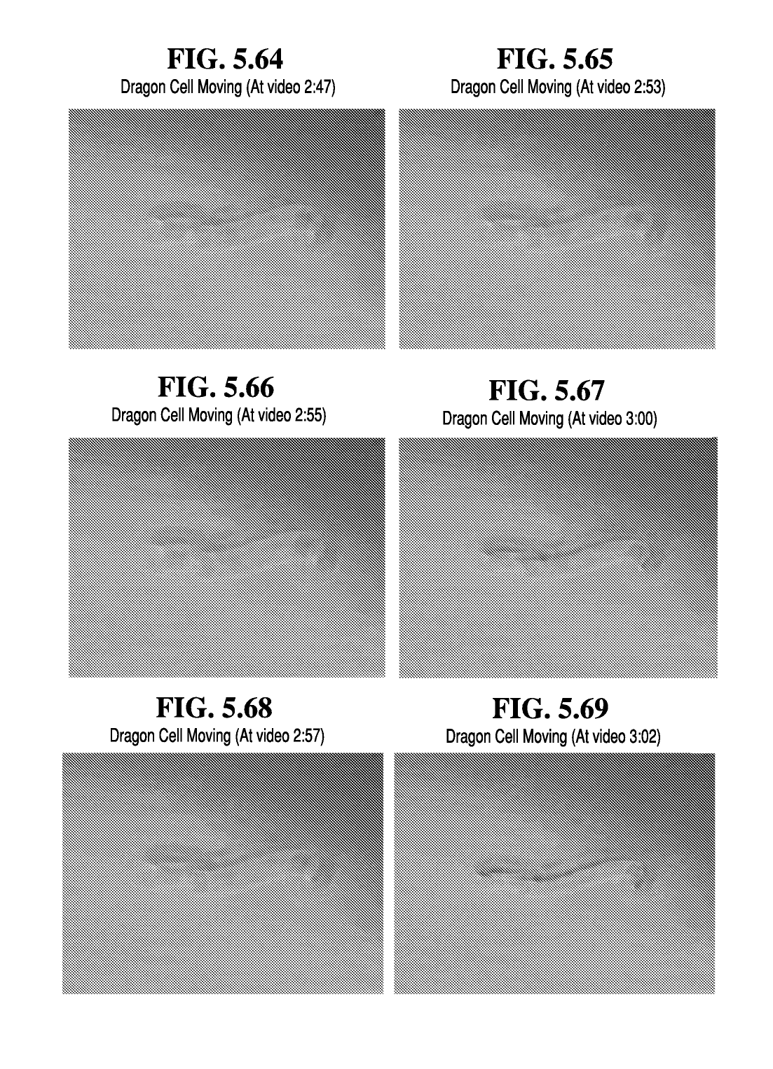

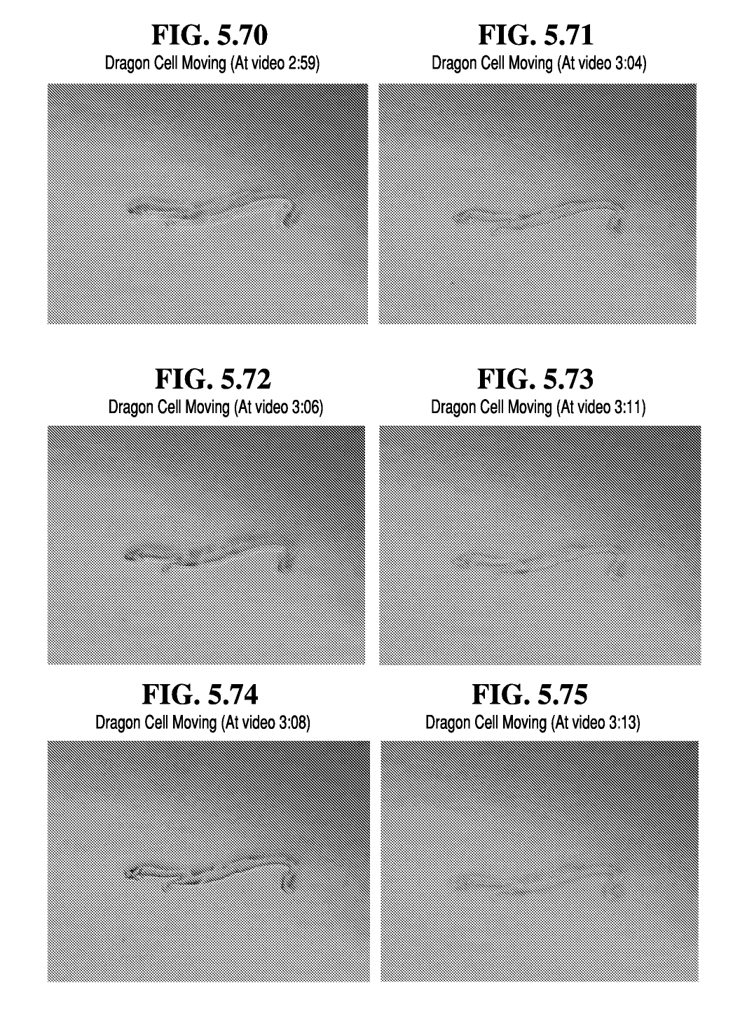

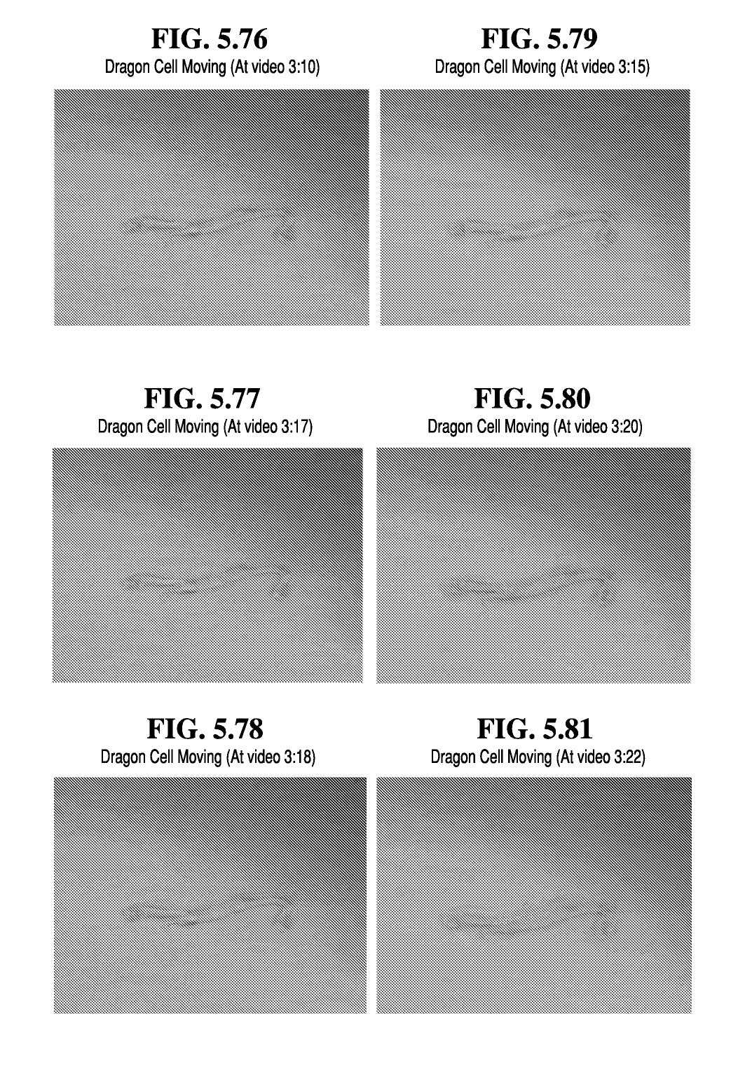

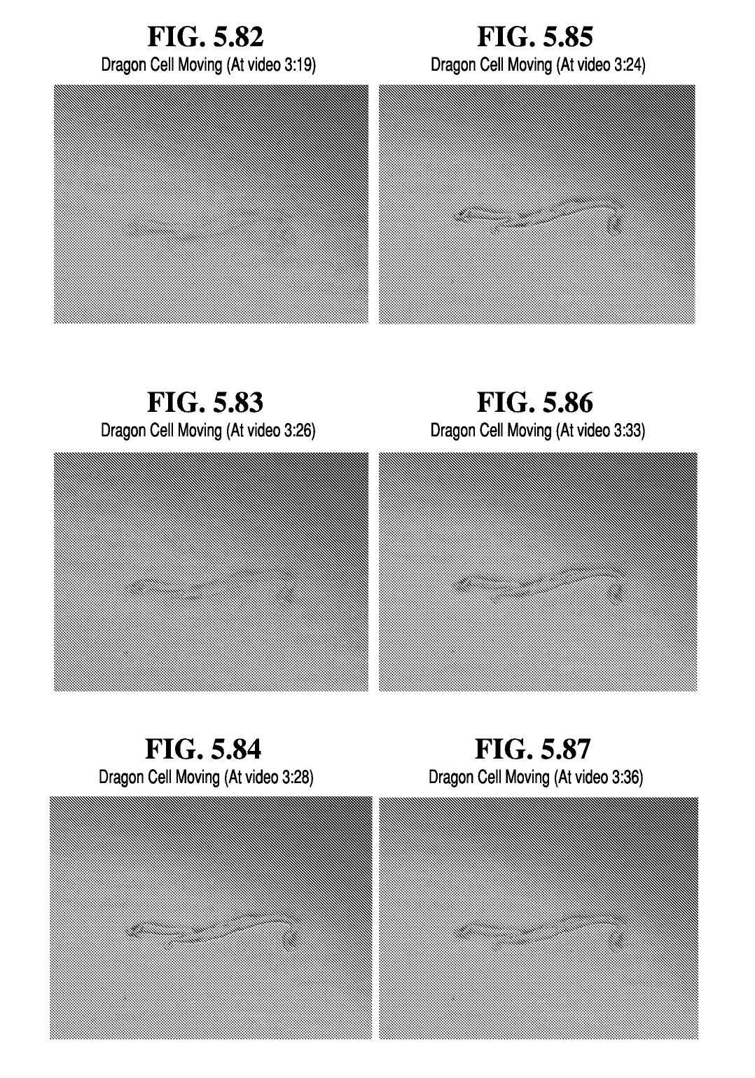

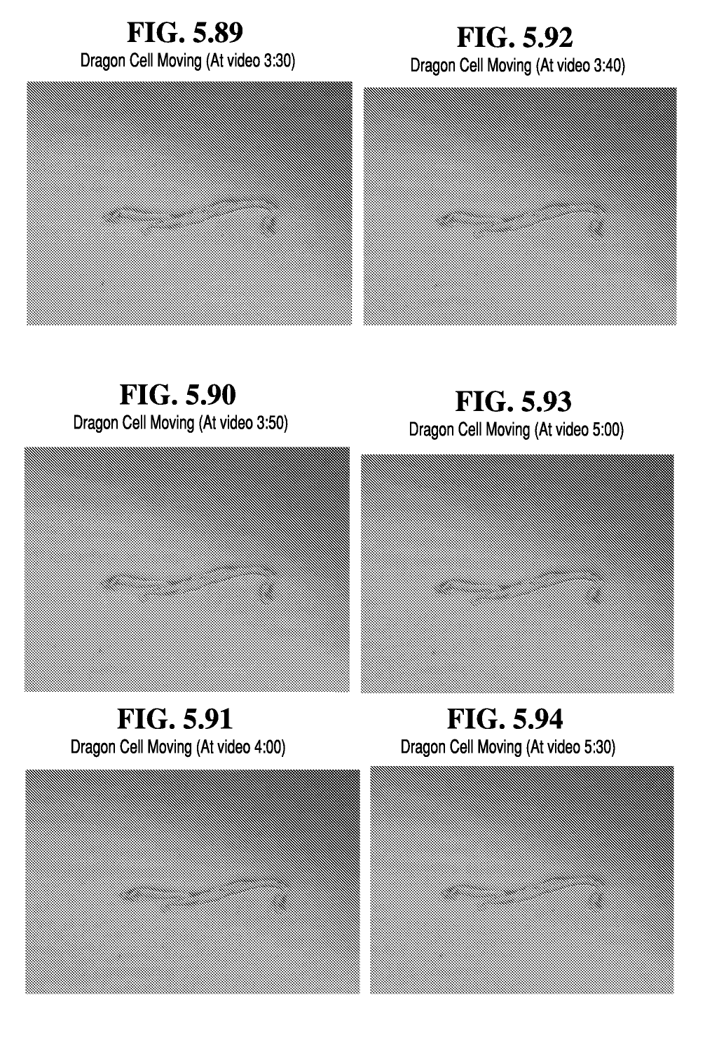

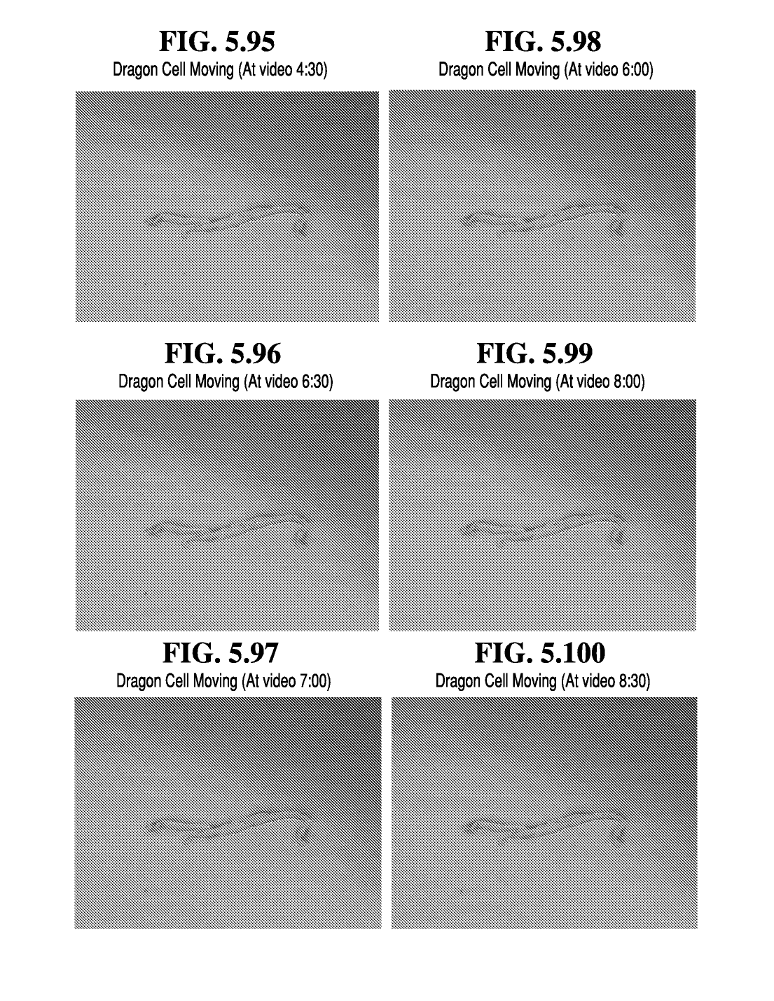

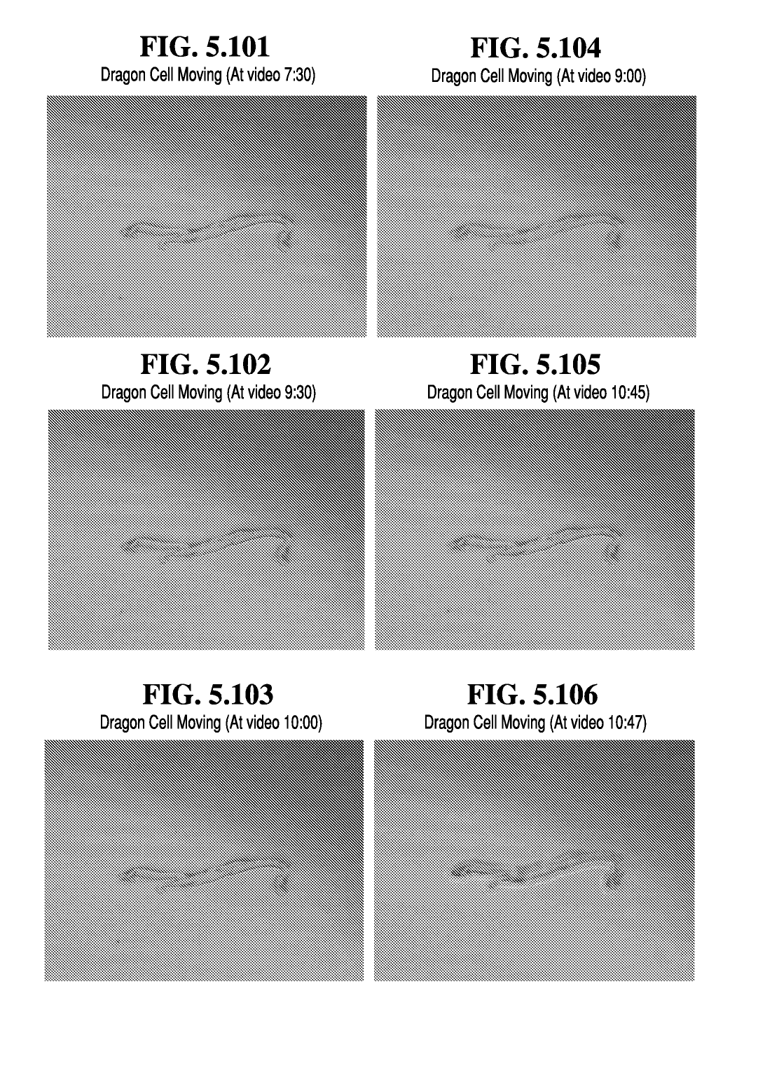

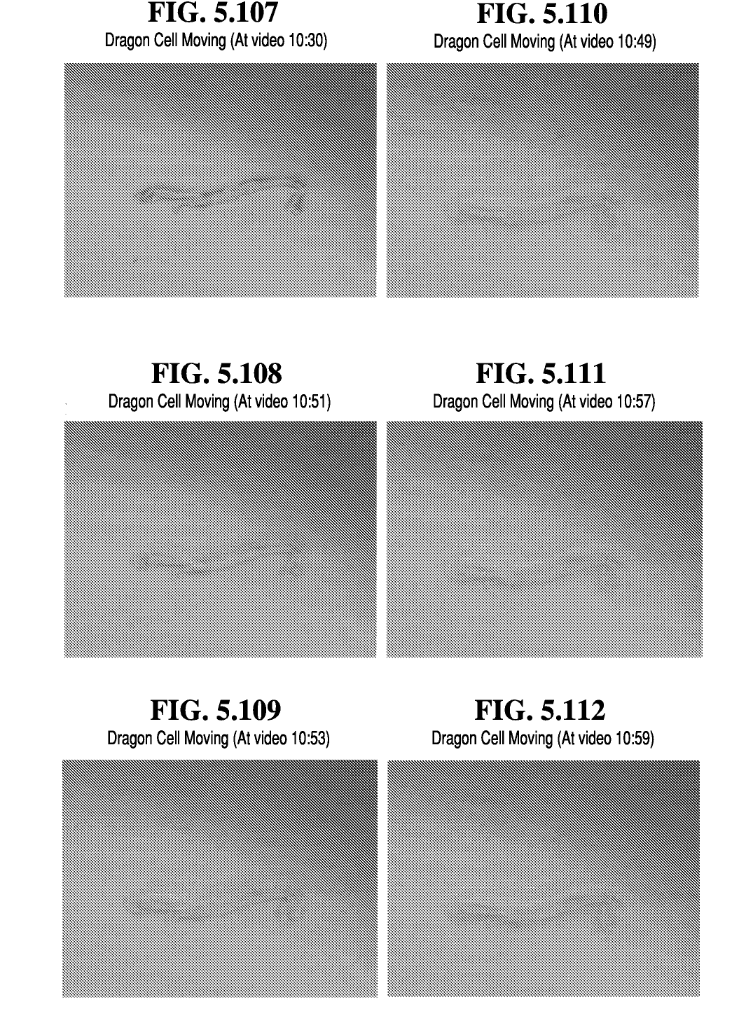

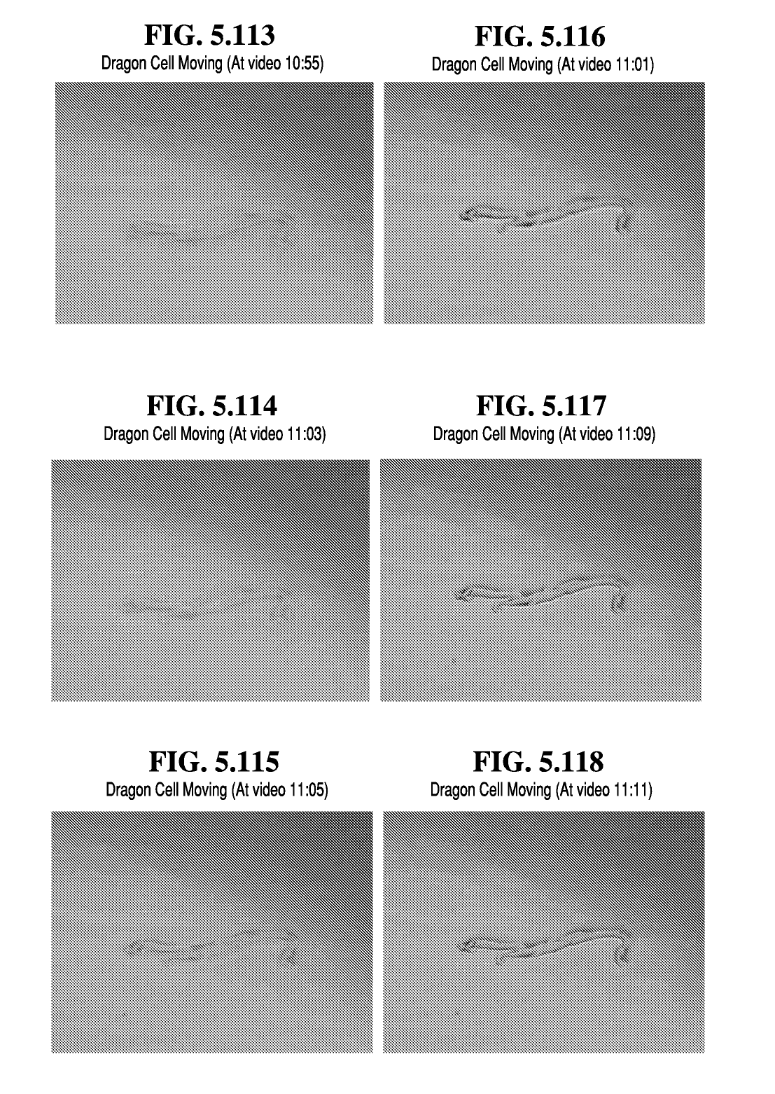

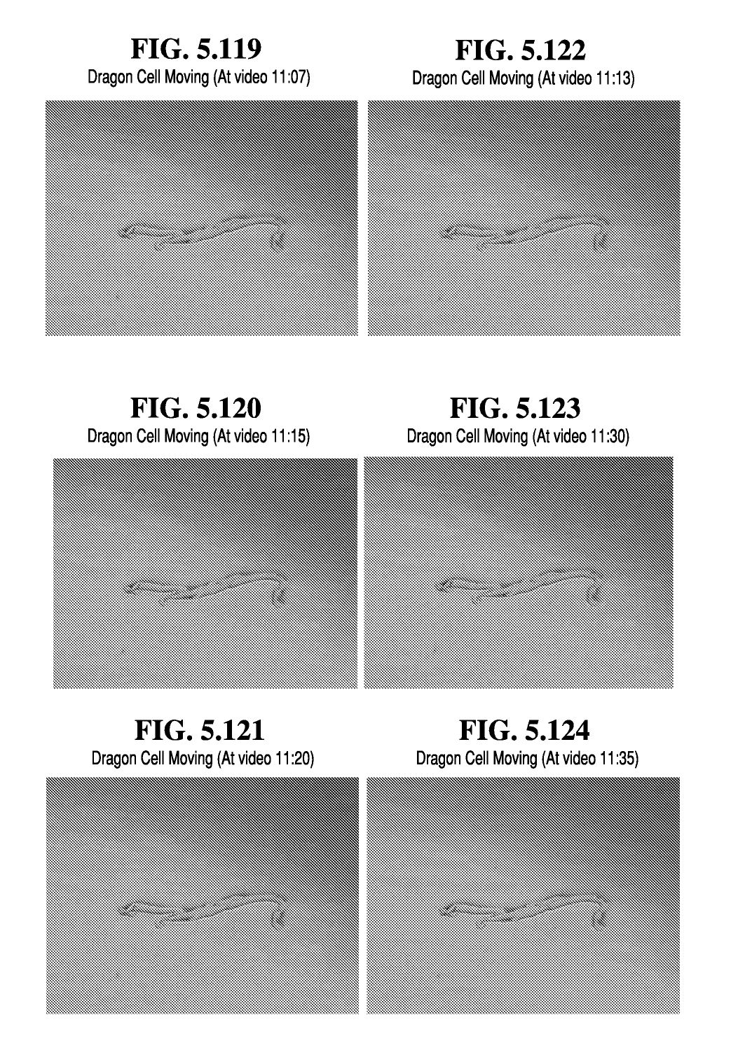

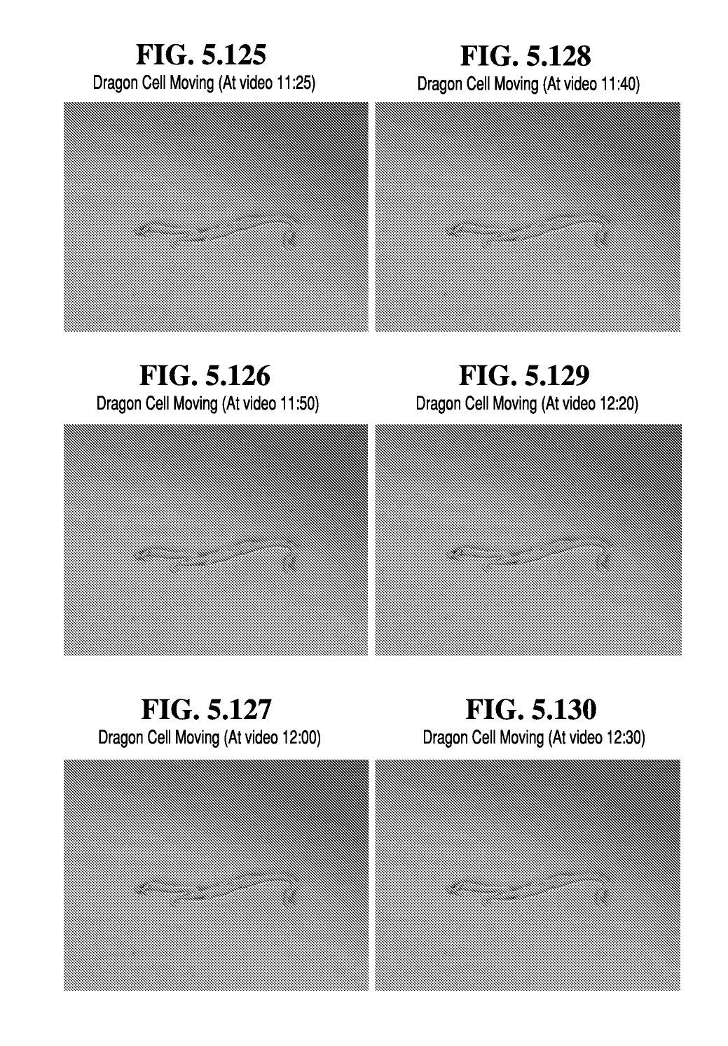

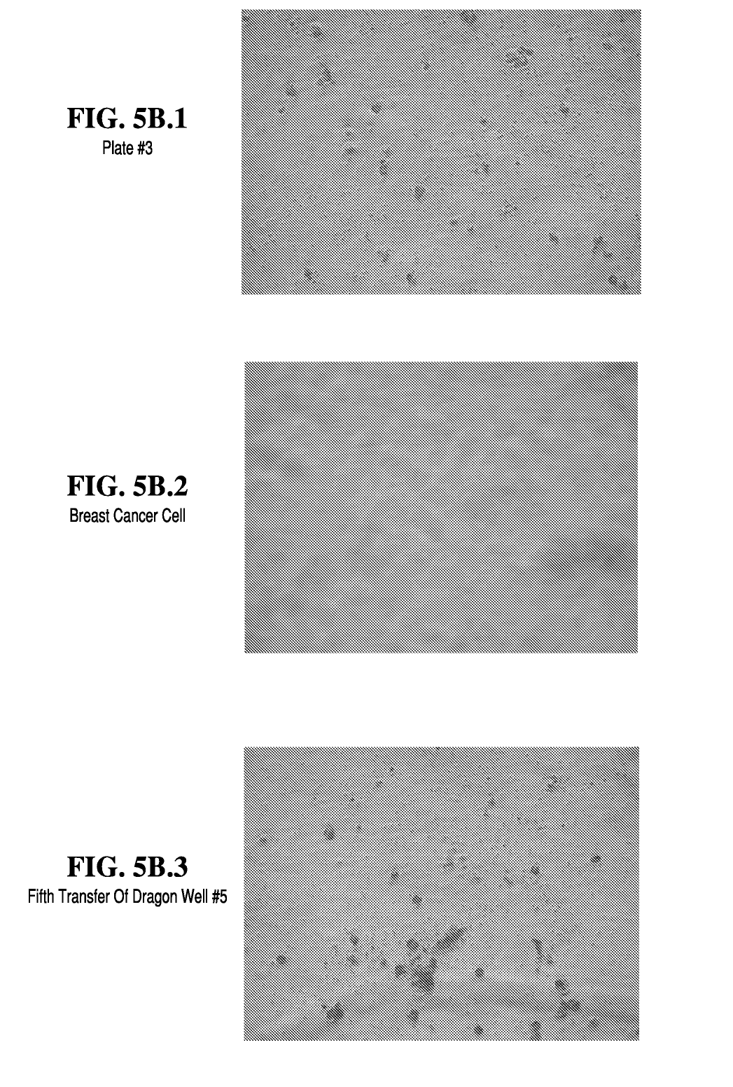

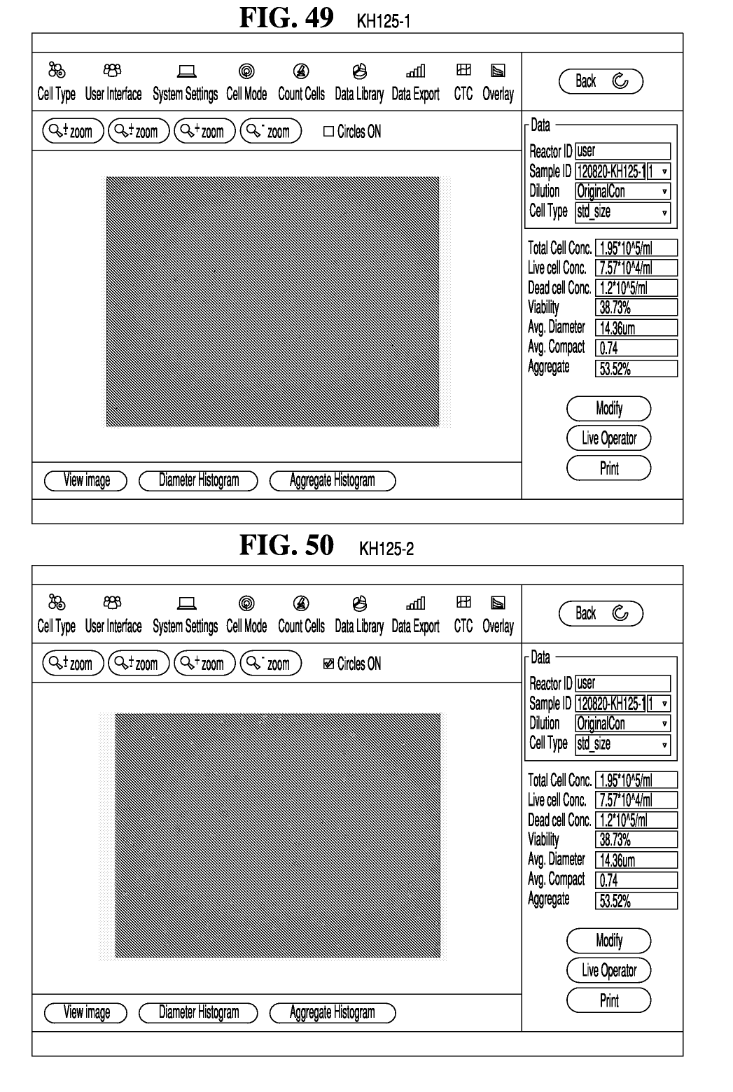

FIGS. 49 and 5—KH126 consists of 10 g of paste of Water Melon into 120 mL of water for injection.

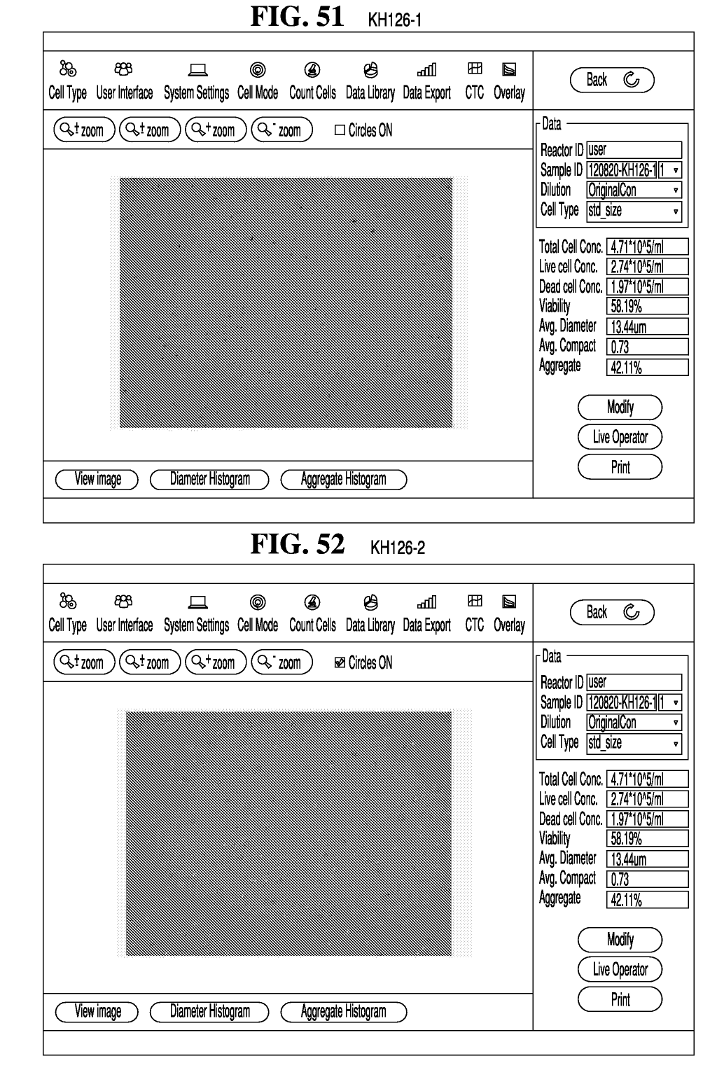

FIGS. 51 and 5—KH127 consists of 34 g of paste of Lychee into 500 mL of water for injection.

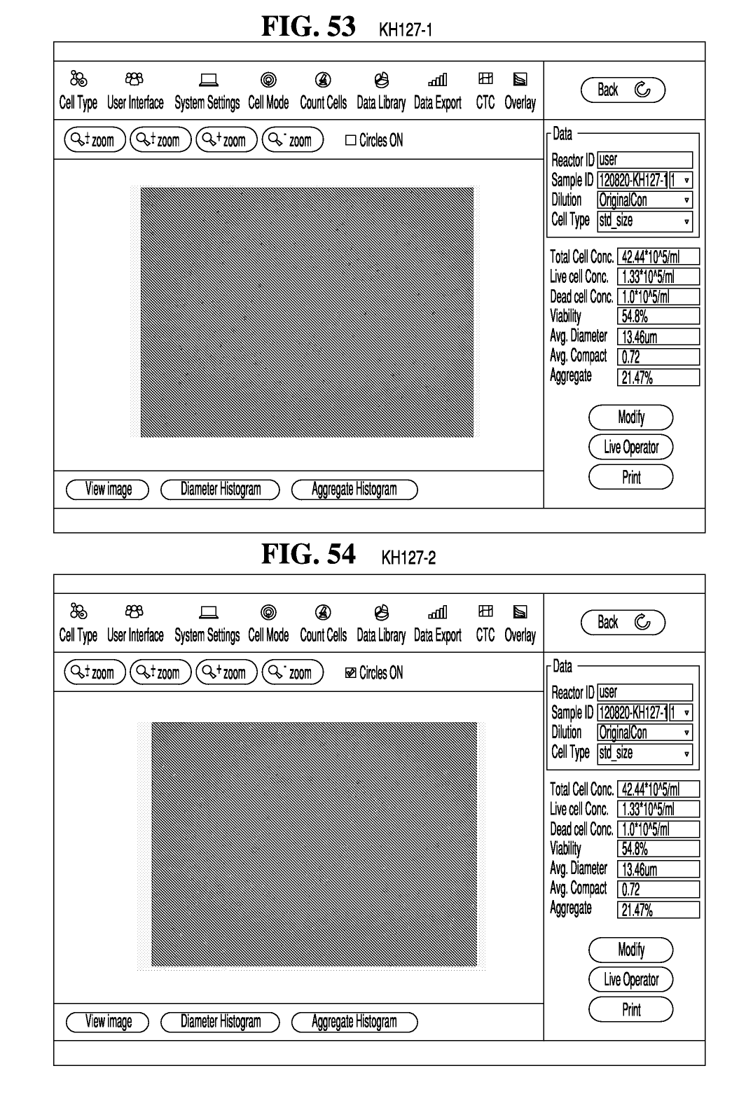

FIGS. 53 and 5—KH128 consists of 15 g of paste of Yellow Melon into 300 mL of water for injection.

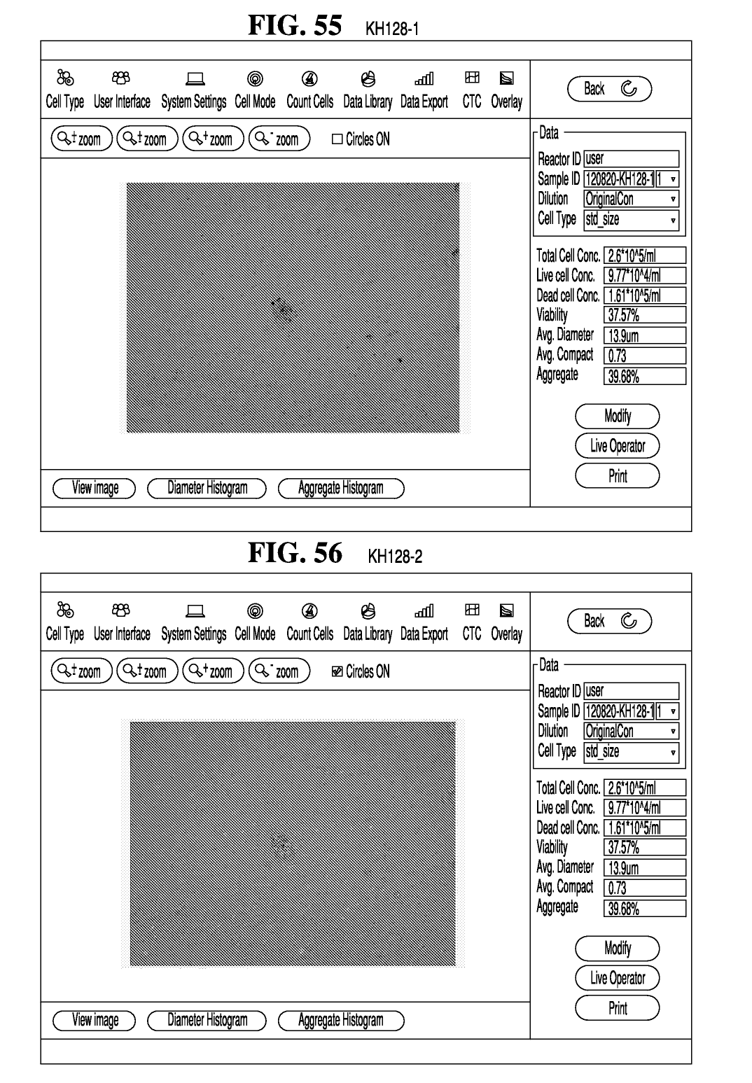

FIGS. 55 and 5—KH129 consists of 21 g of paste of Pineapple into 350 mL of water for injection.

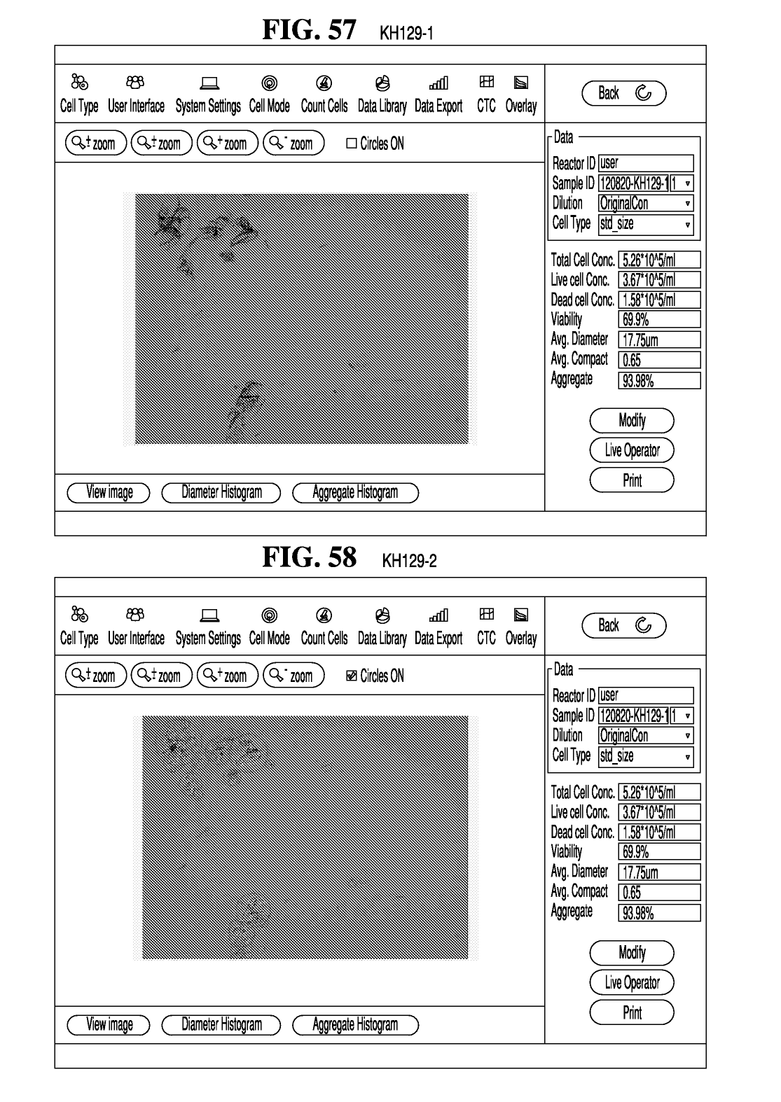

FIGS. 57 and 5—KH130 consists of 10 bottles of coconut juice.

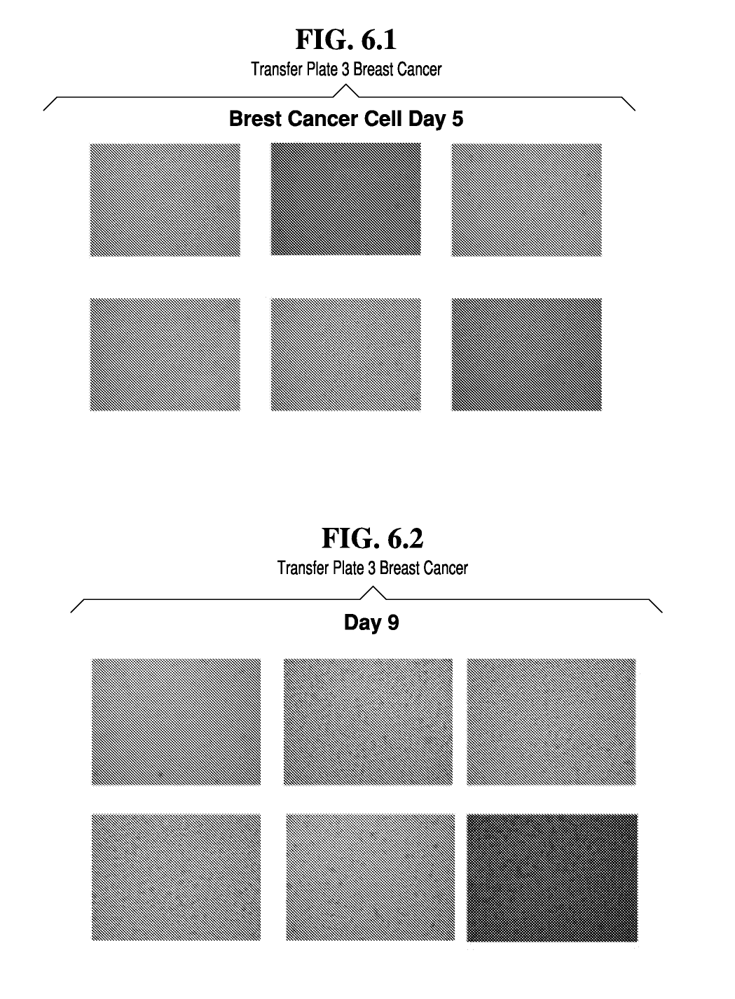

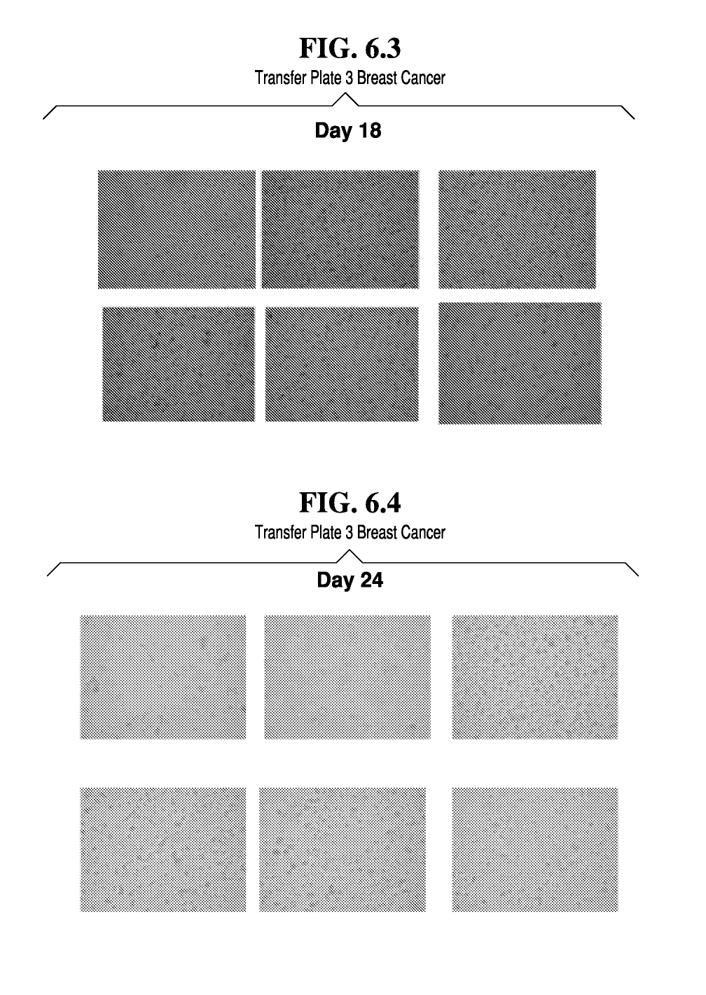

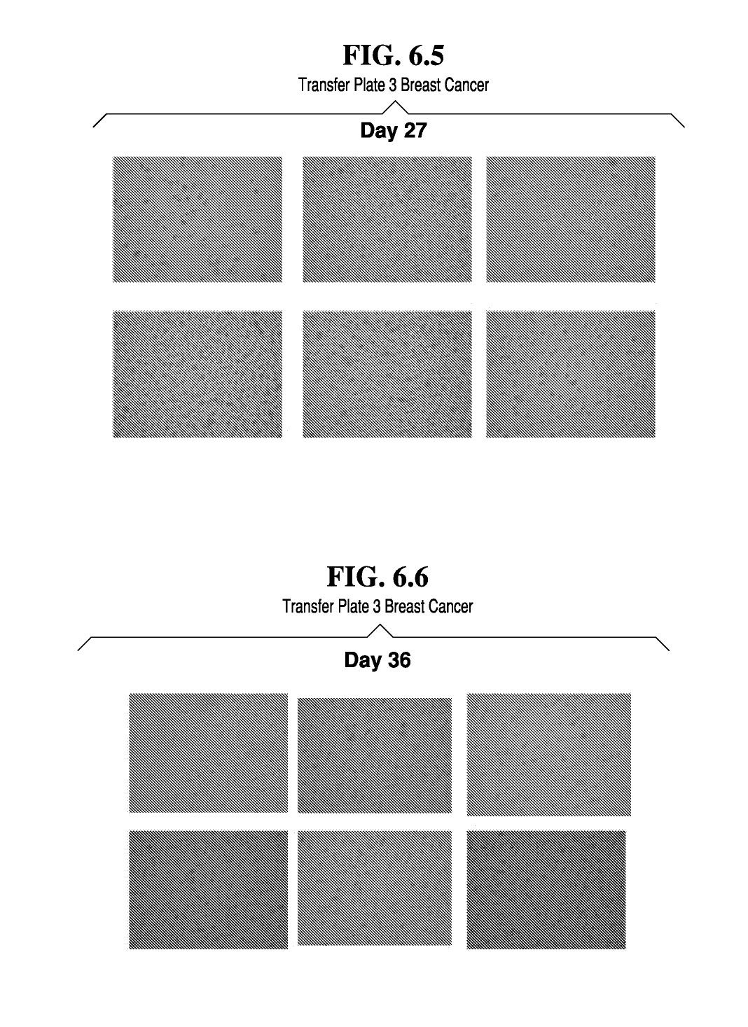

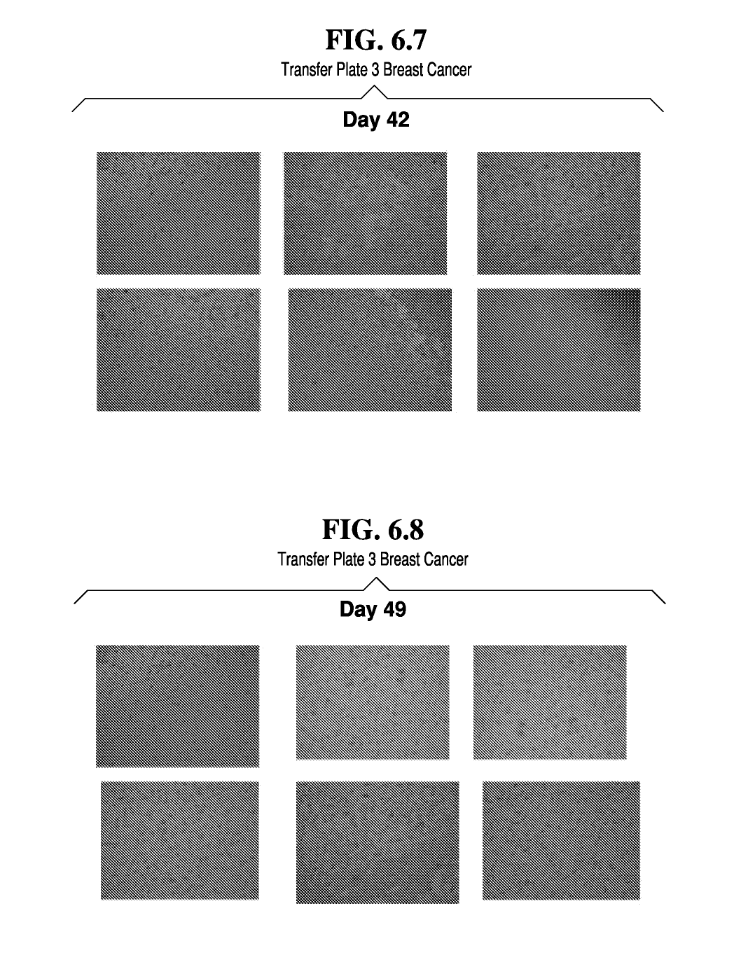

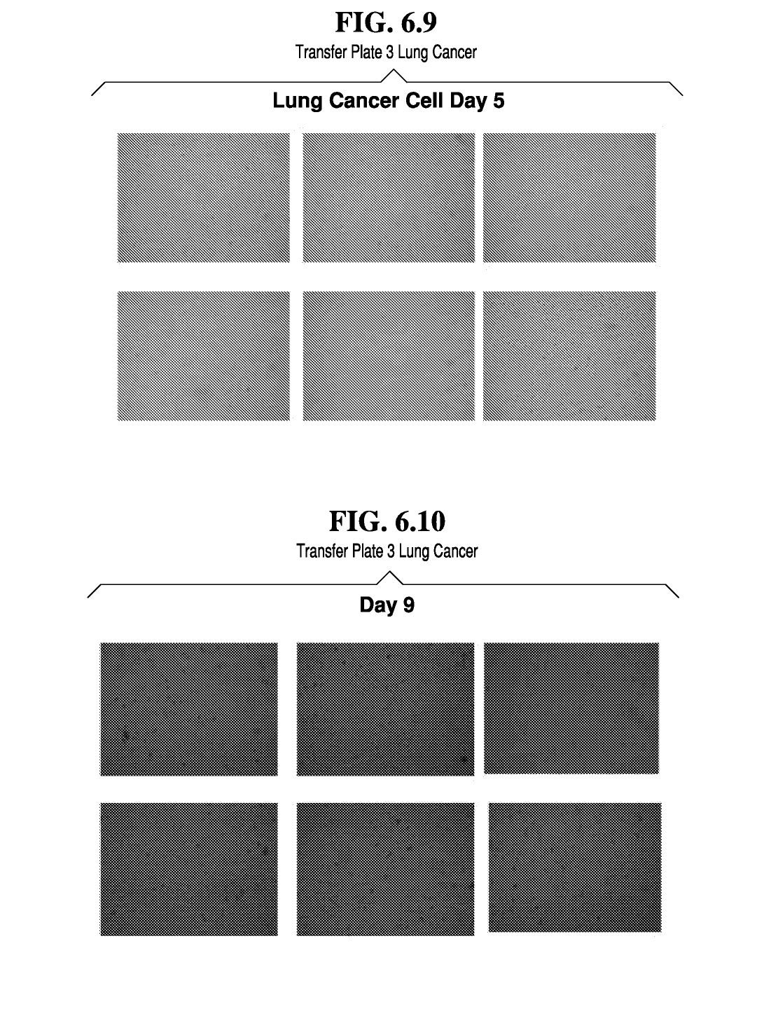

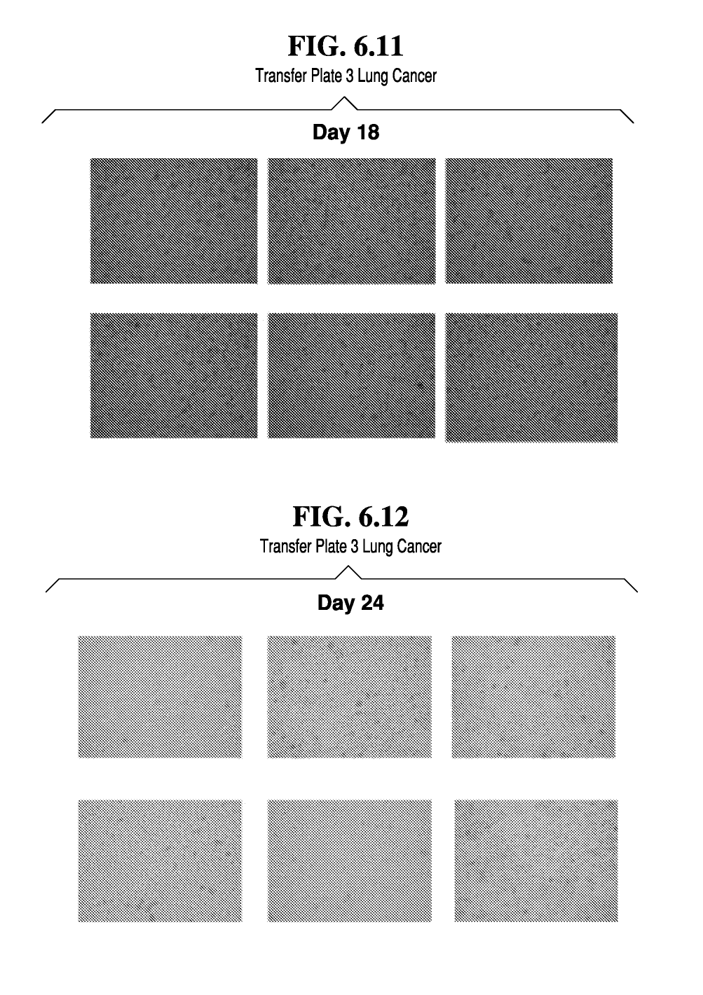

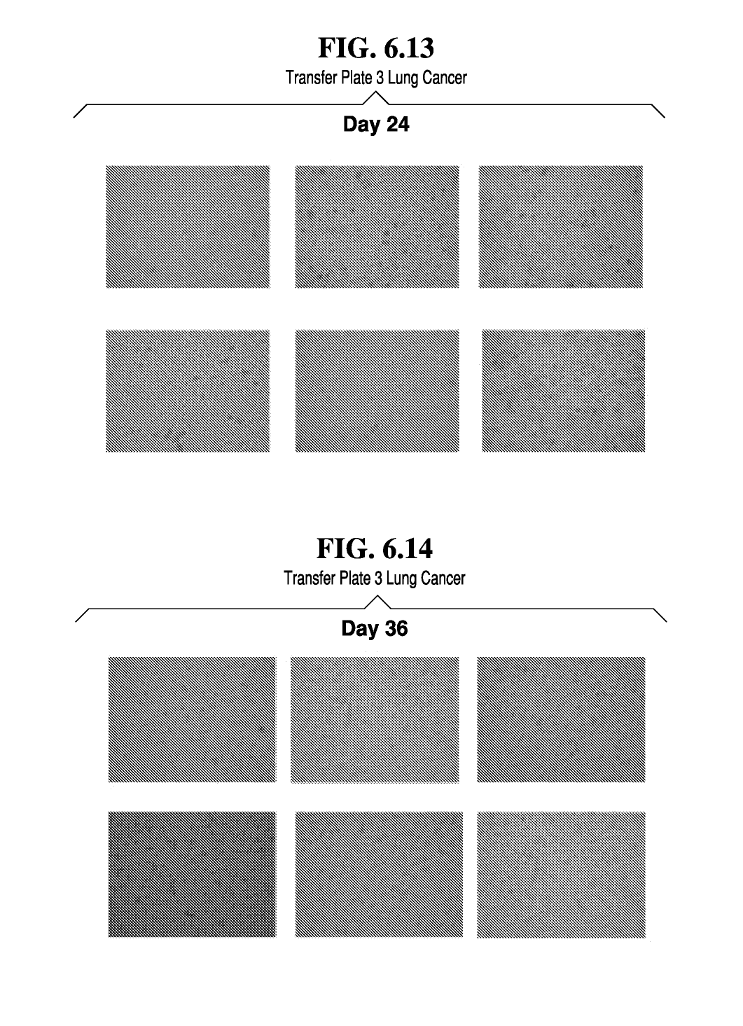

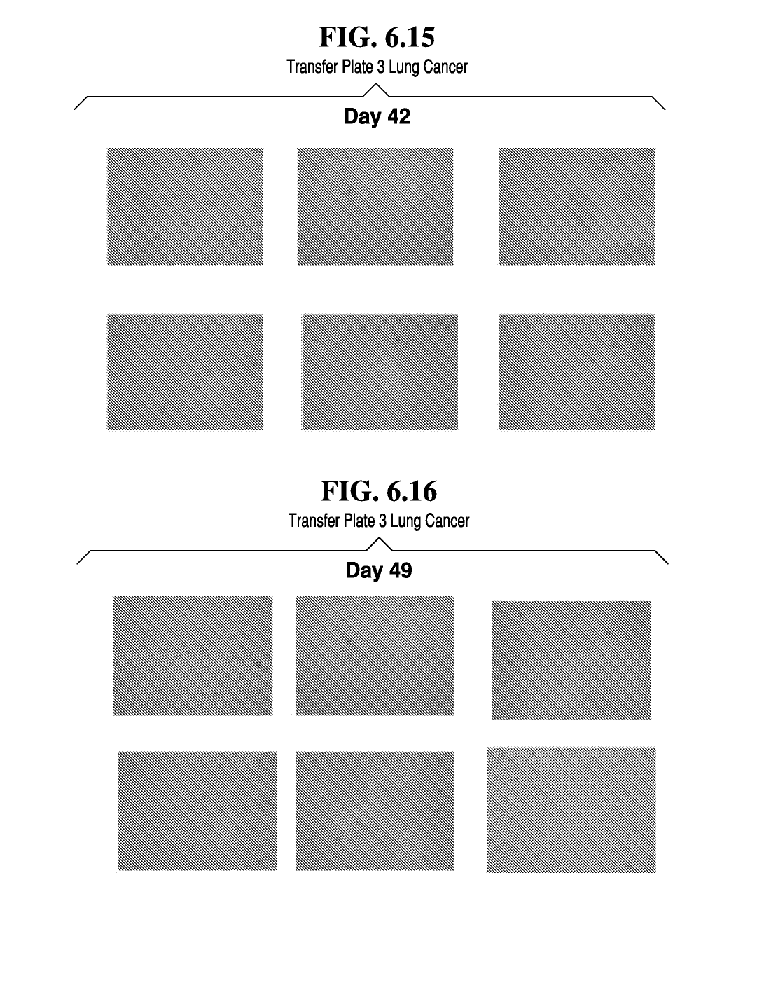

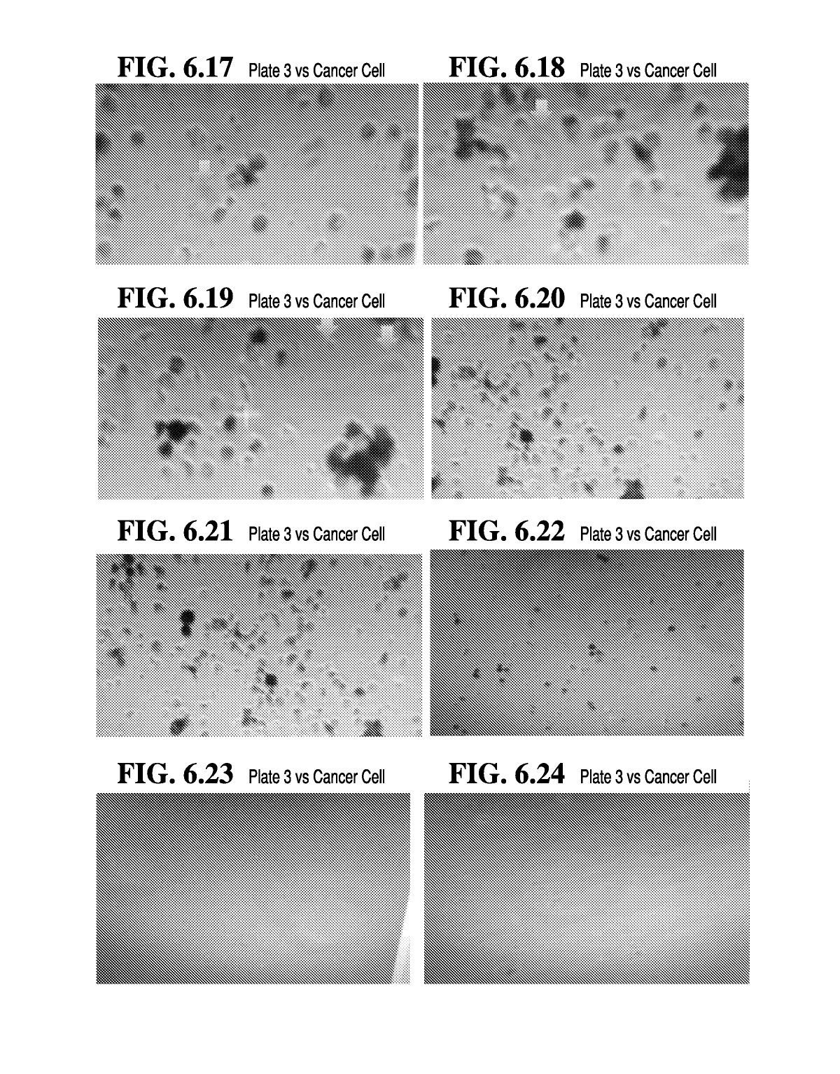

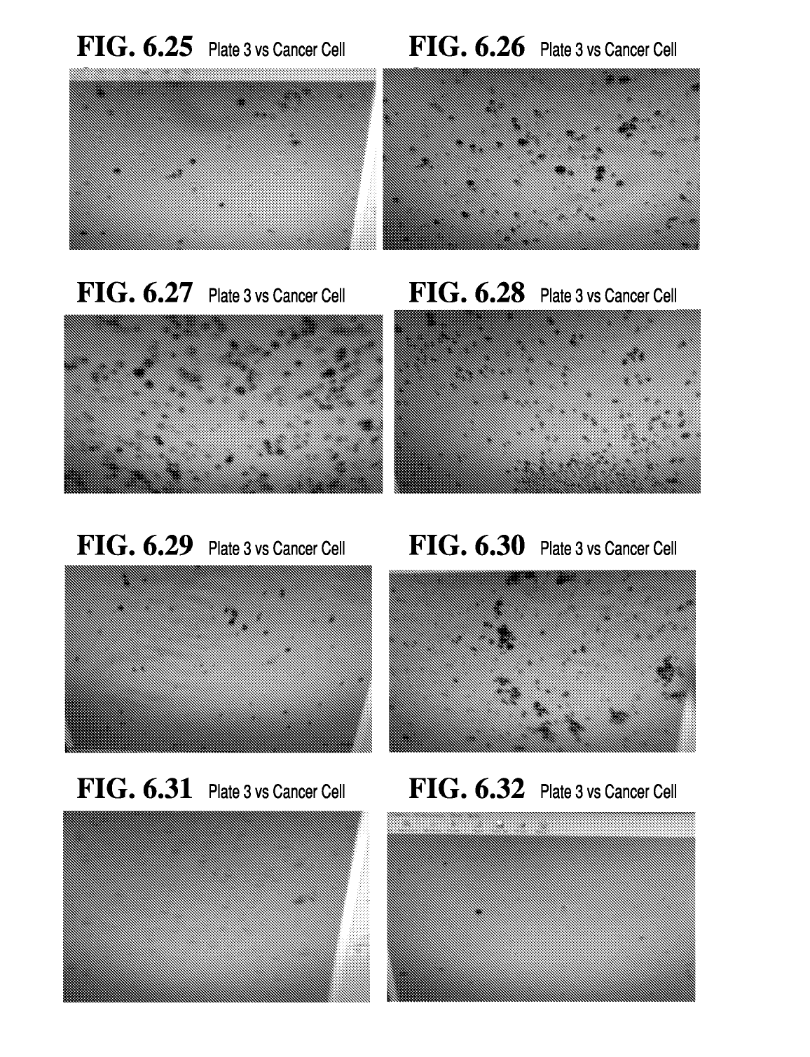

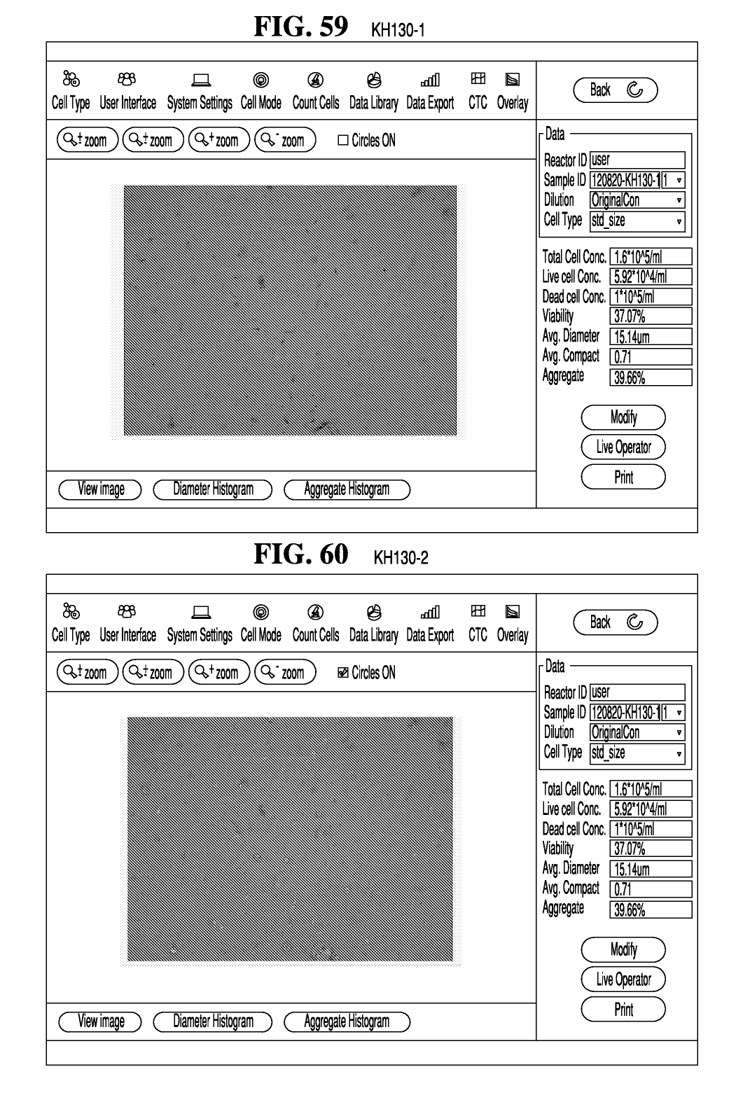

FIGS. 59 and 6—KH131 consists of Mint

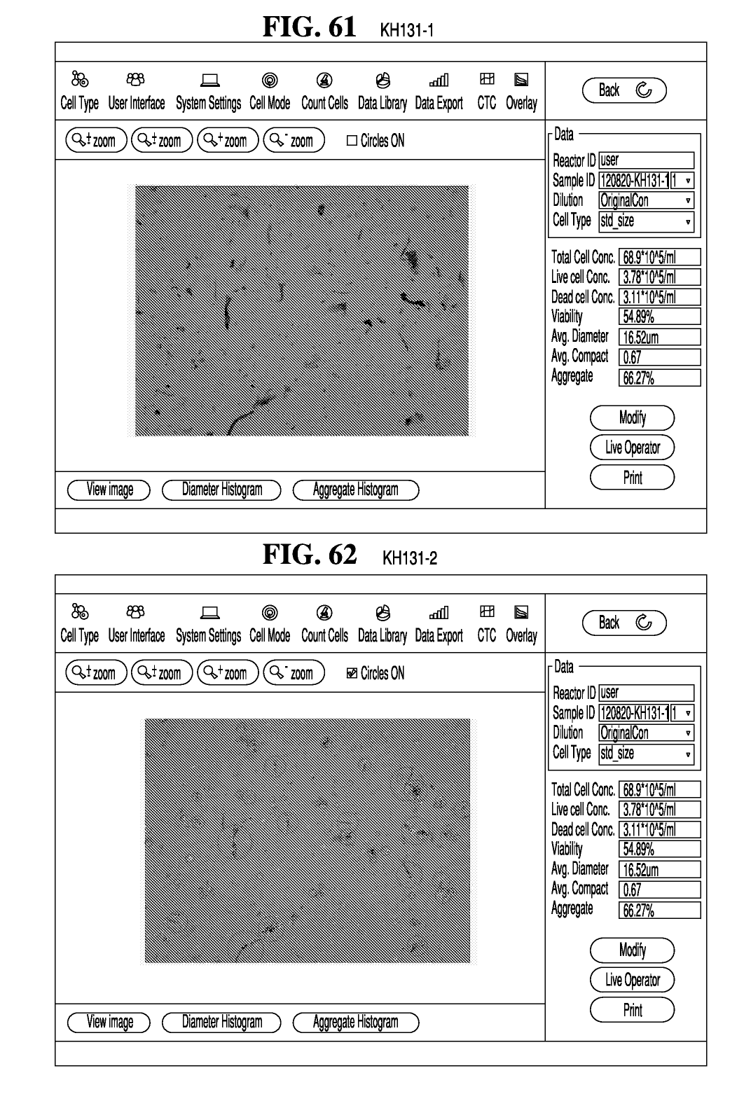

FIGS. 61 and 6—KH132 consists of Hot Pepper

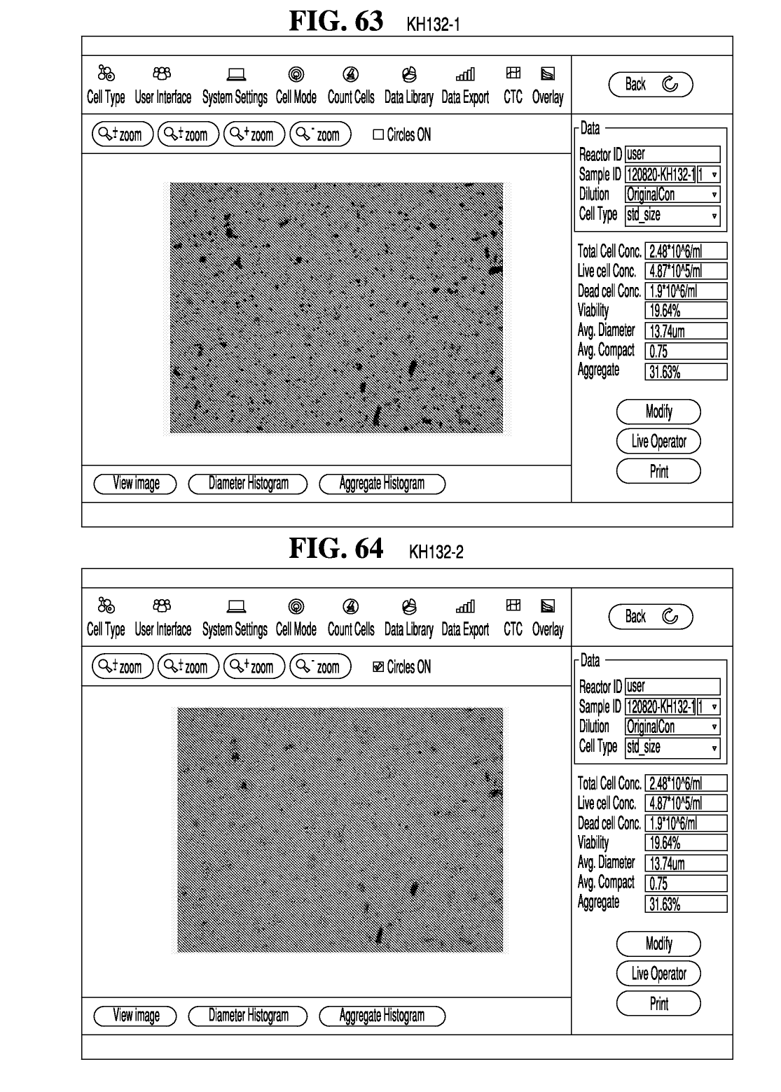

FIGS. 63 and 6—KH133 consists of Black Pepper

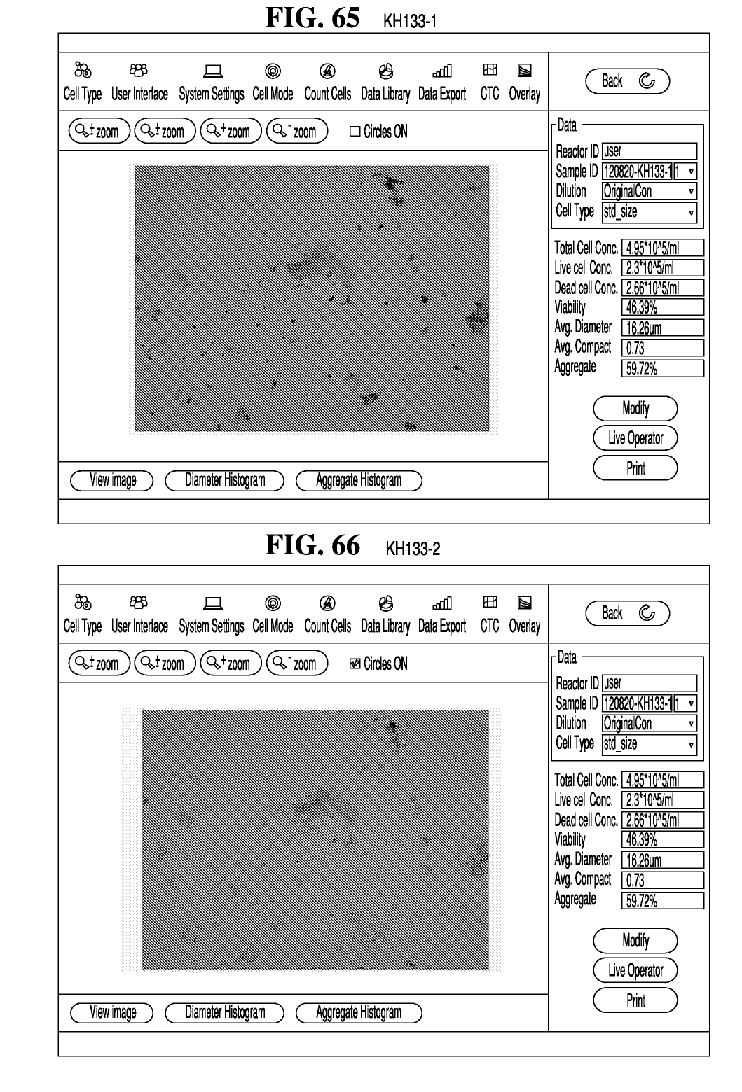

FIGS. 65 and 6—KH134 consists of Carrot

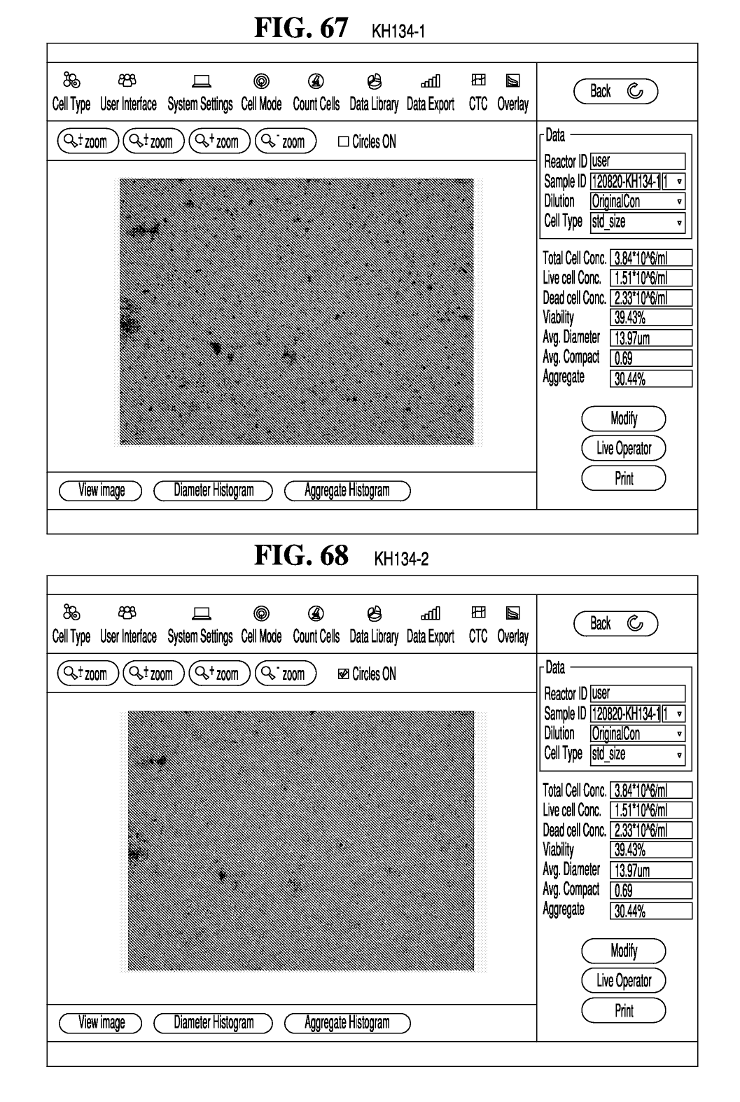

FIGS. 67 and 6—KH135 consists of Banana

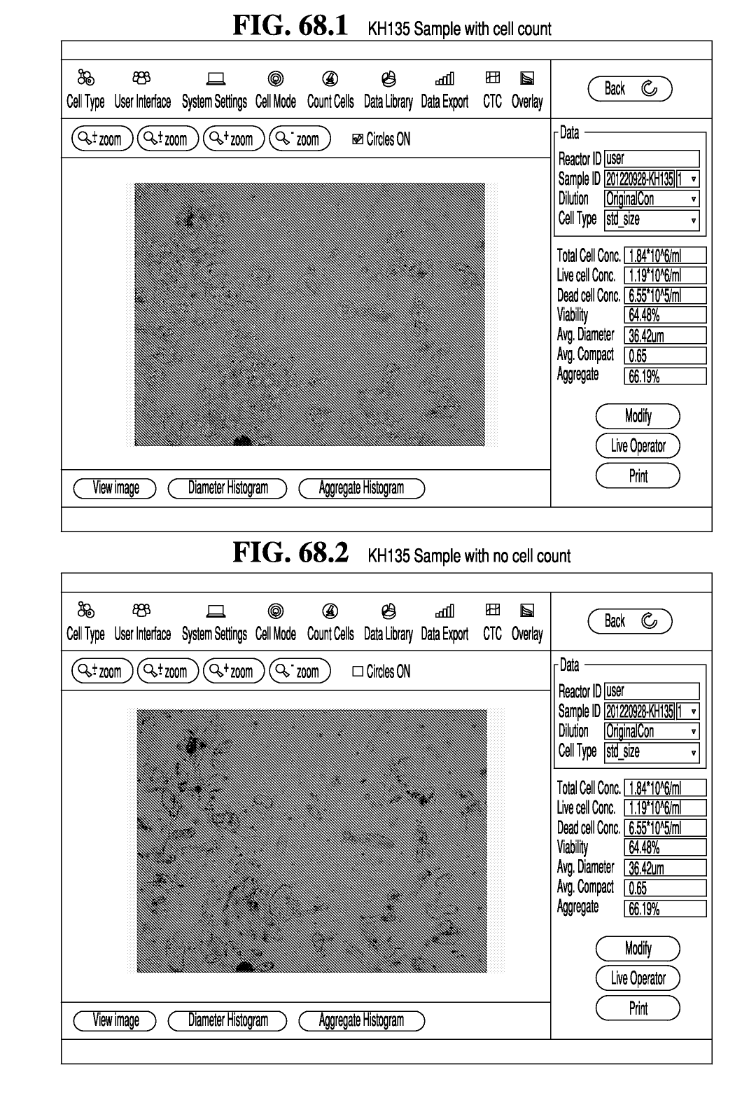

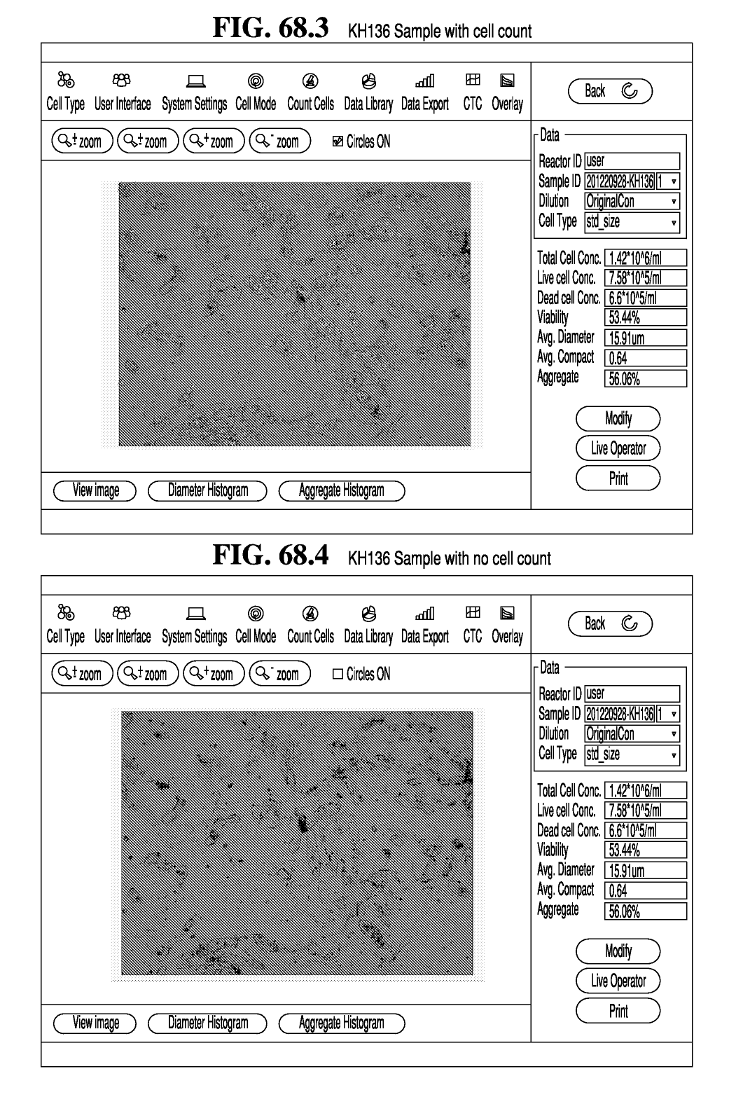

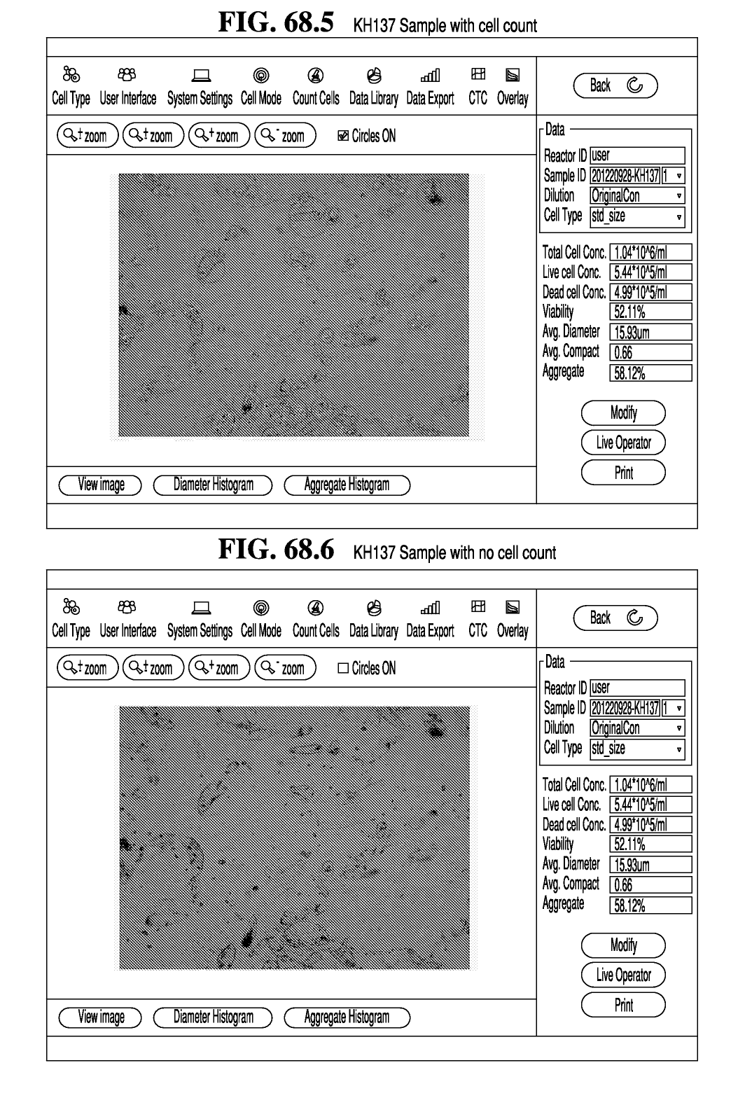

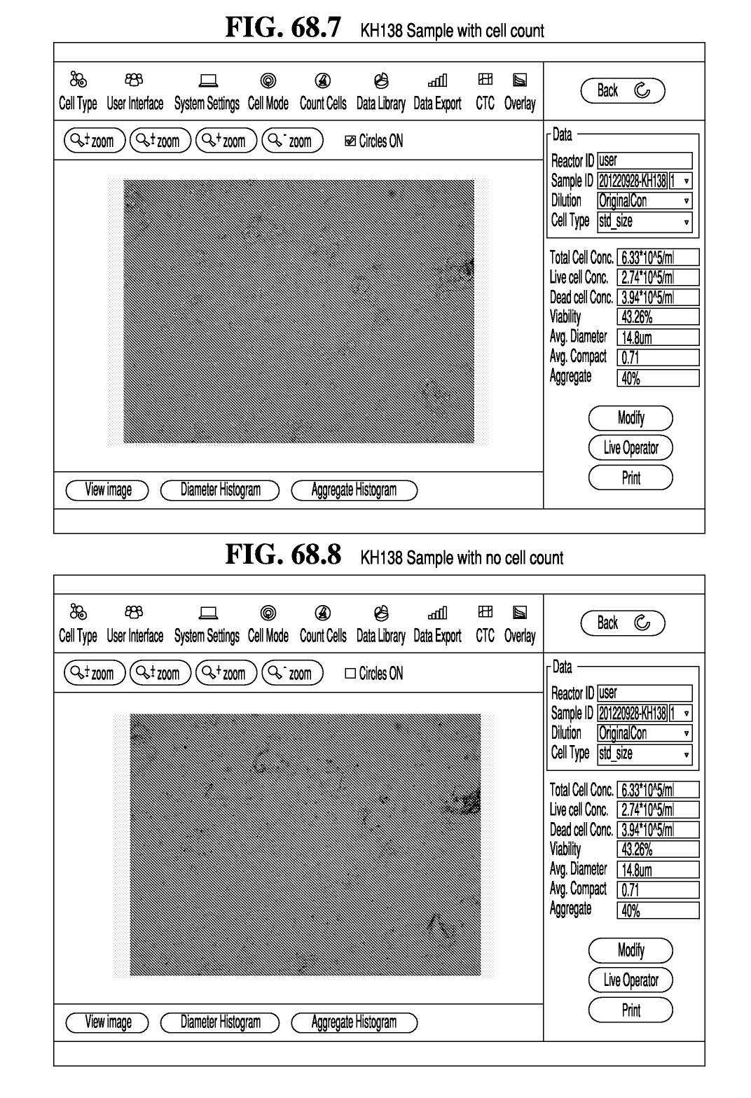

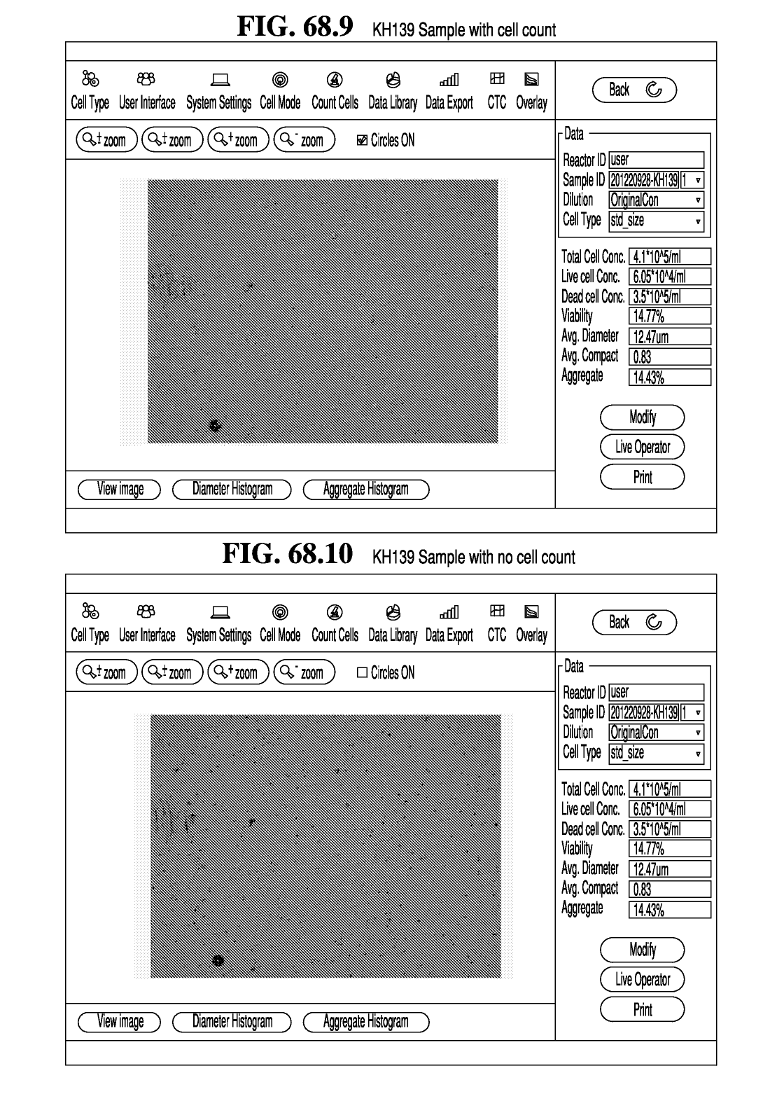

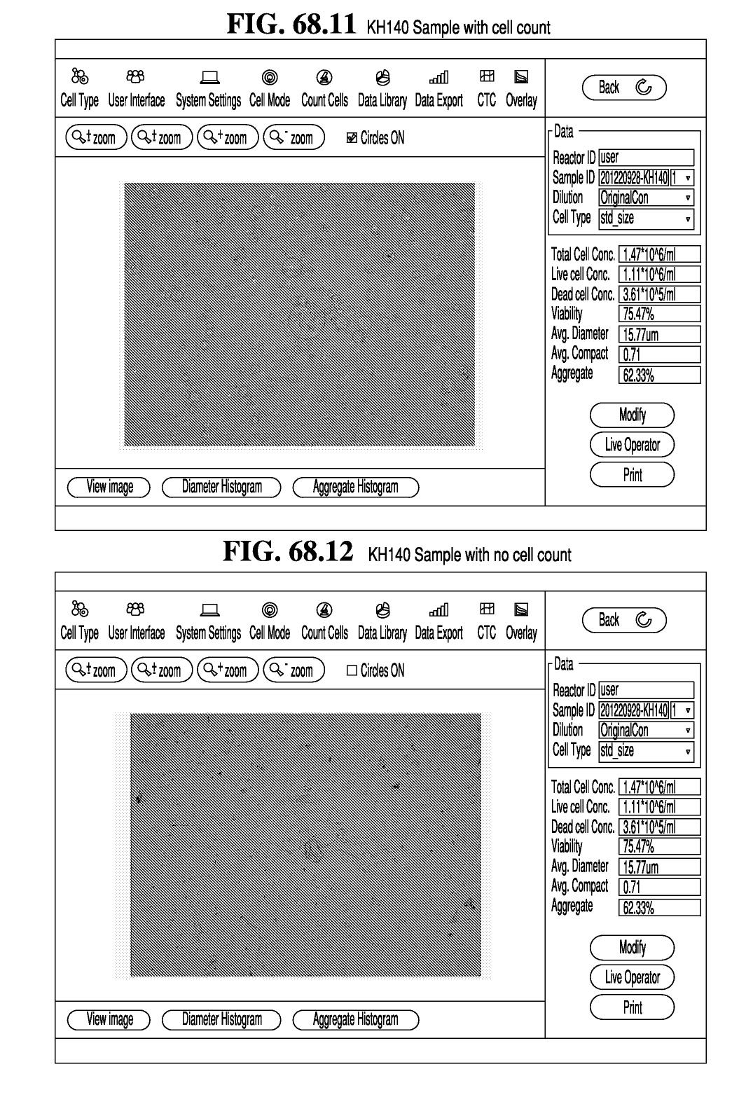

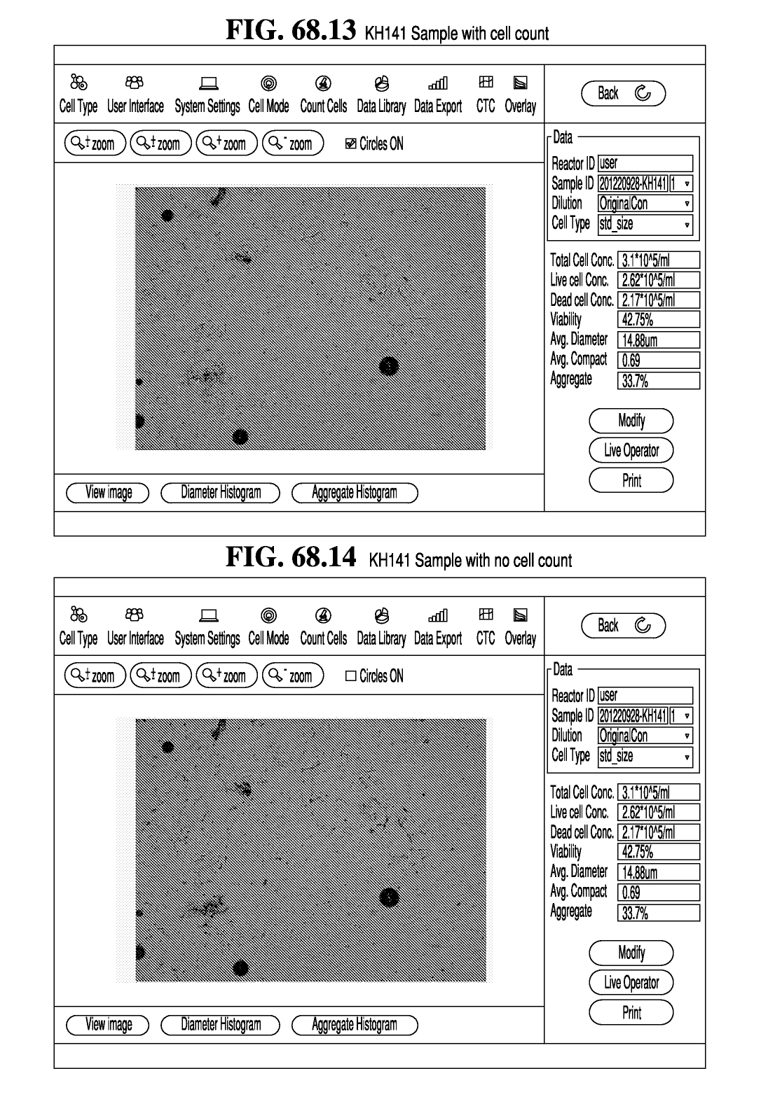

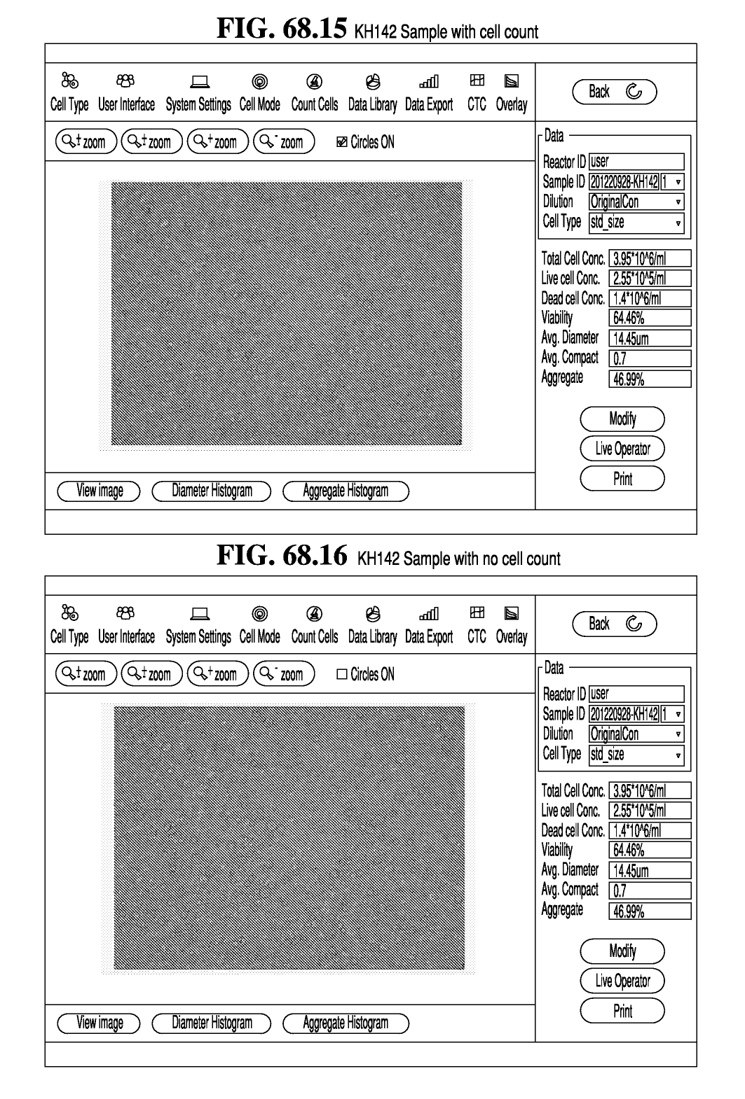

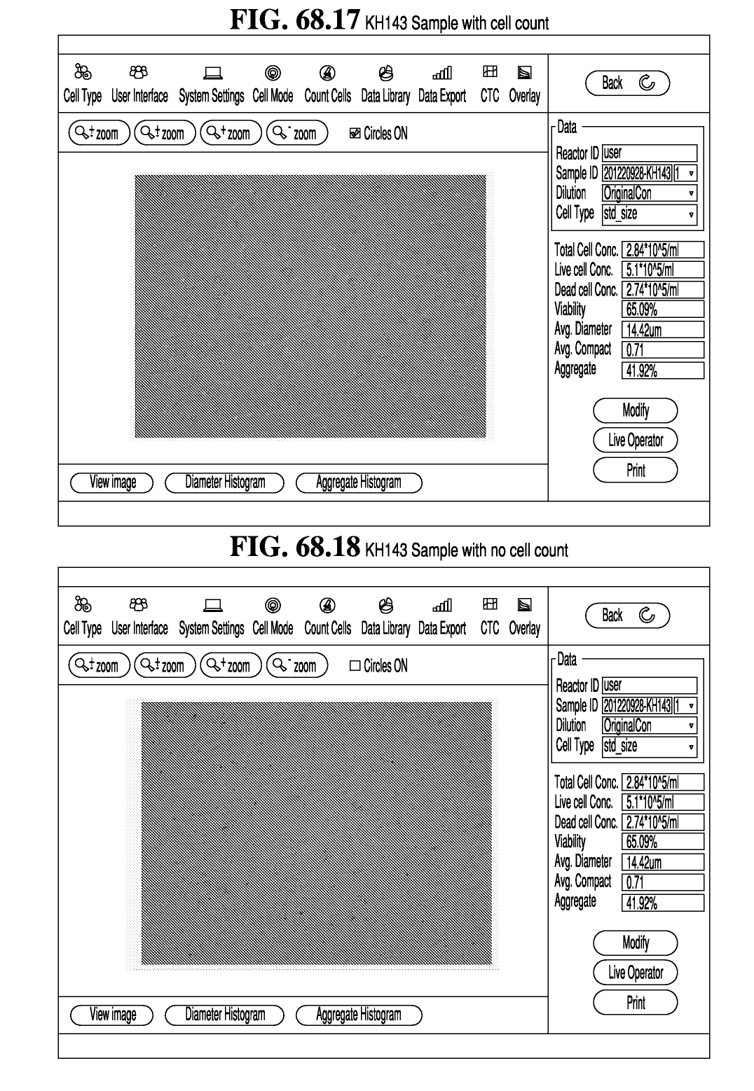

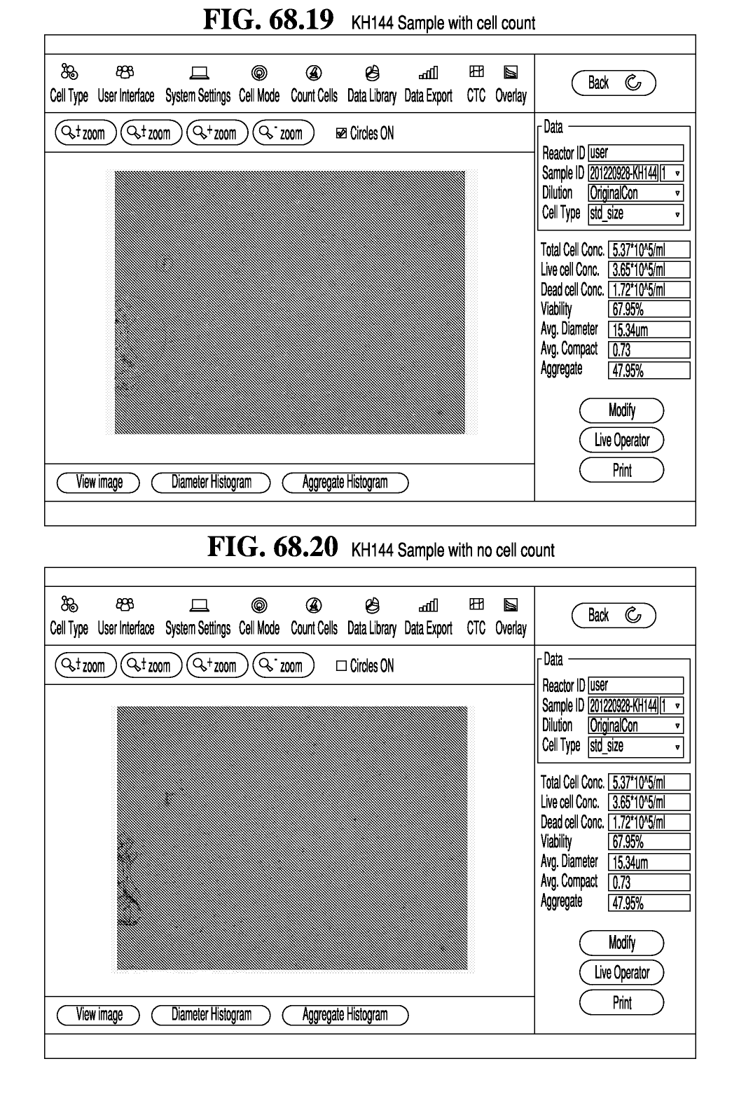

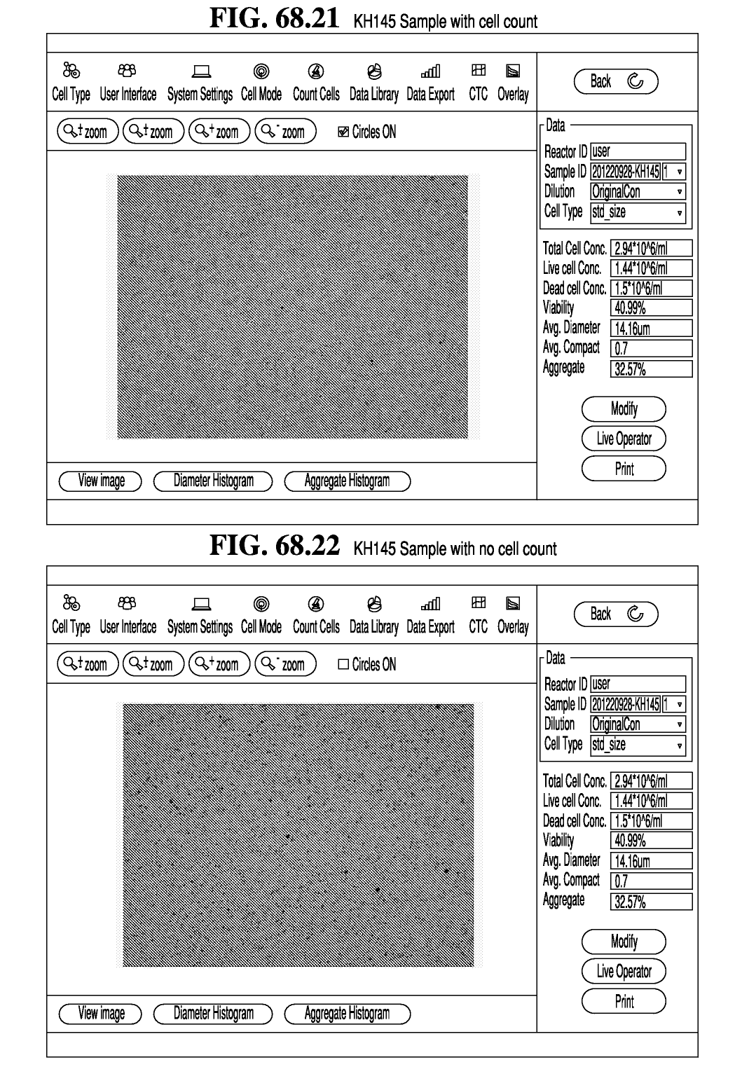

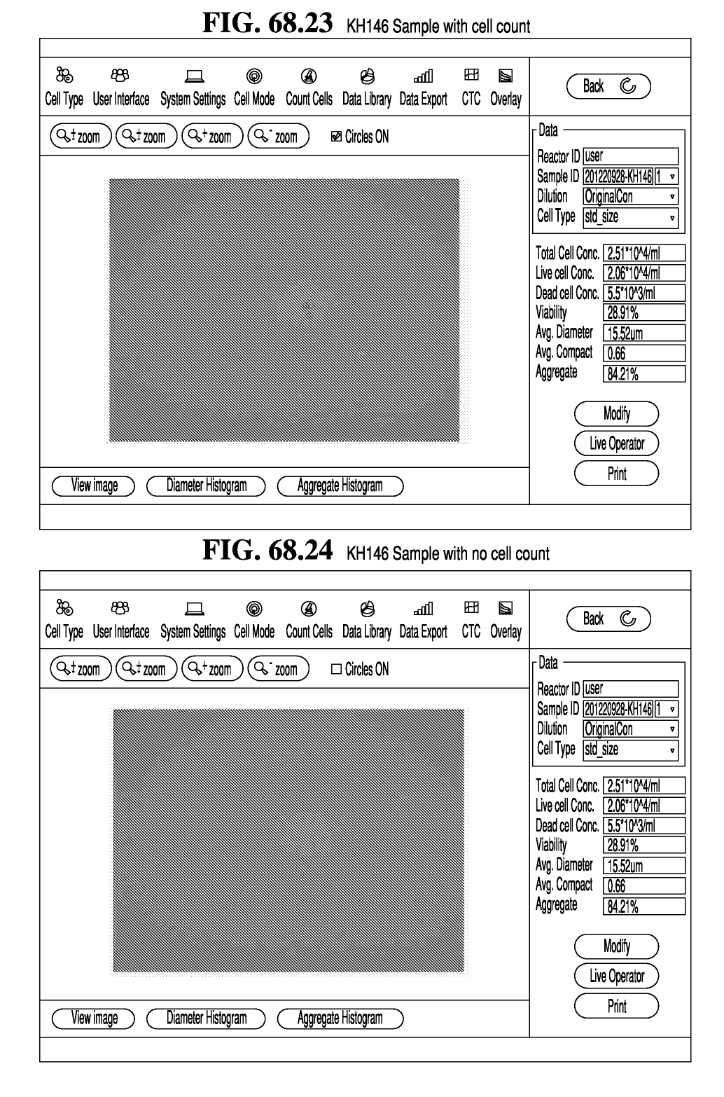

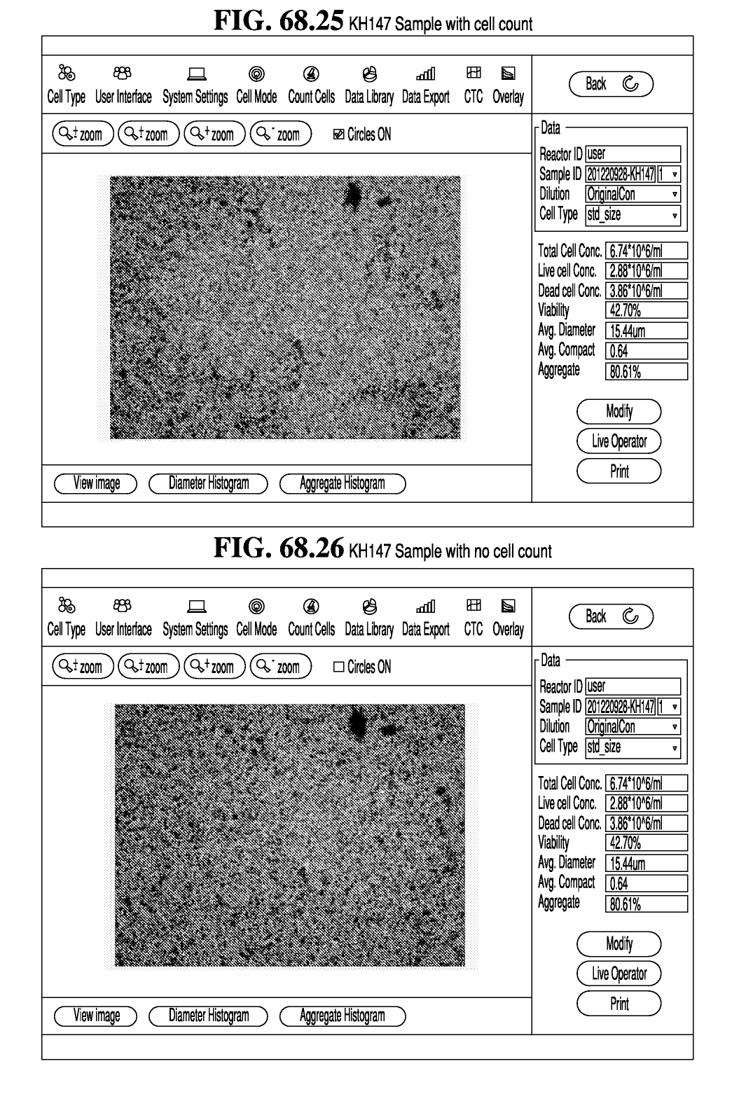

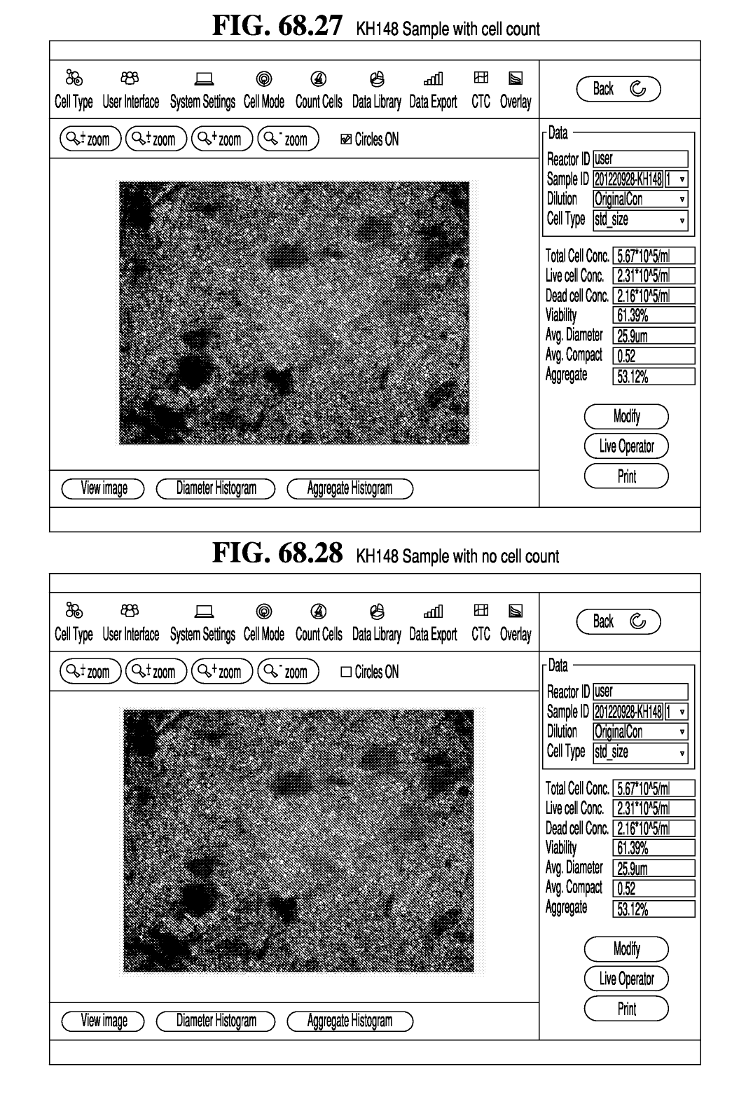

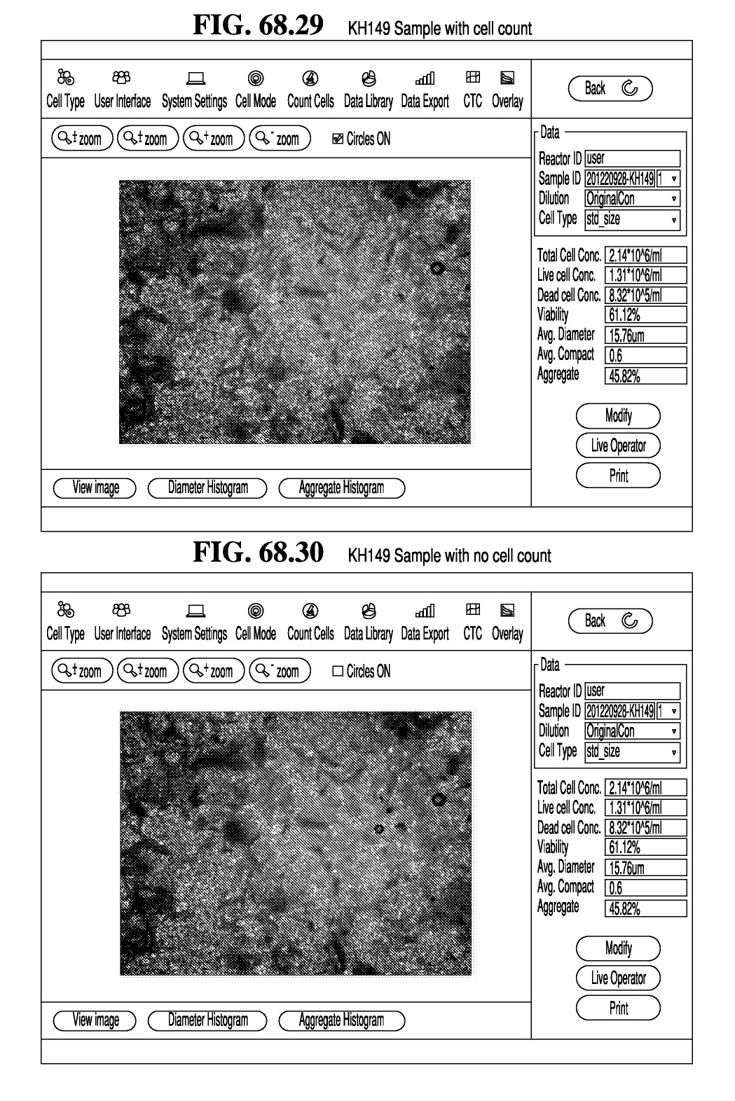

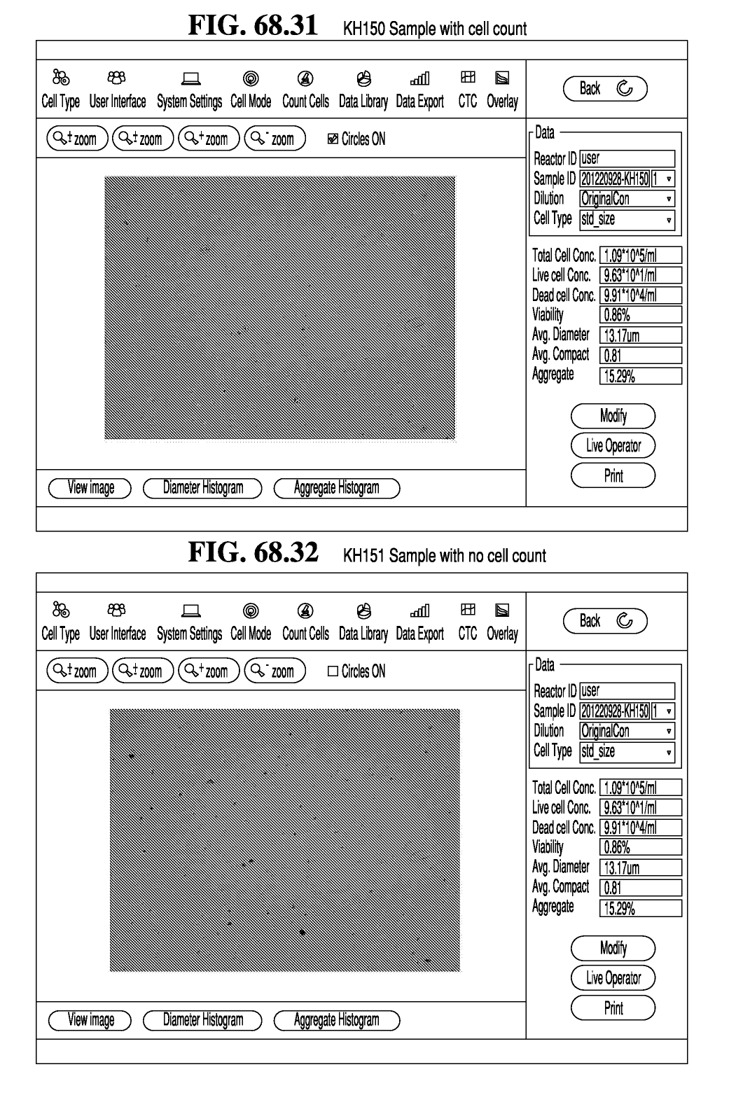

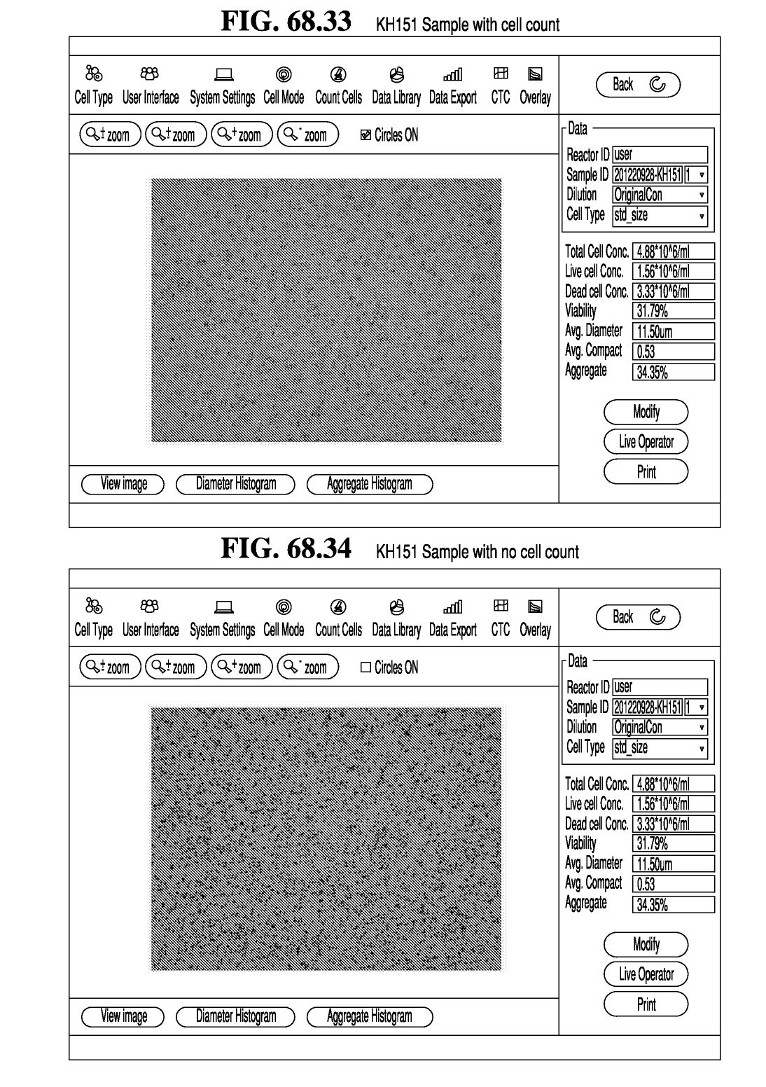

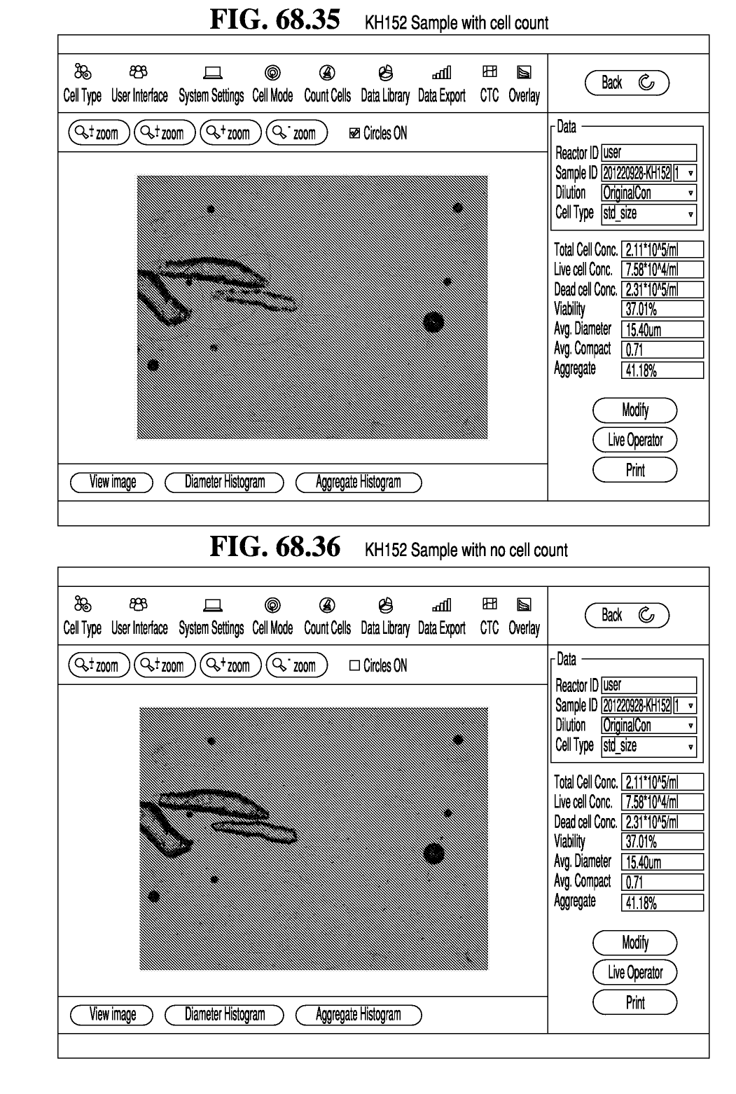

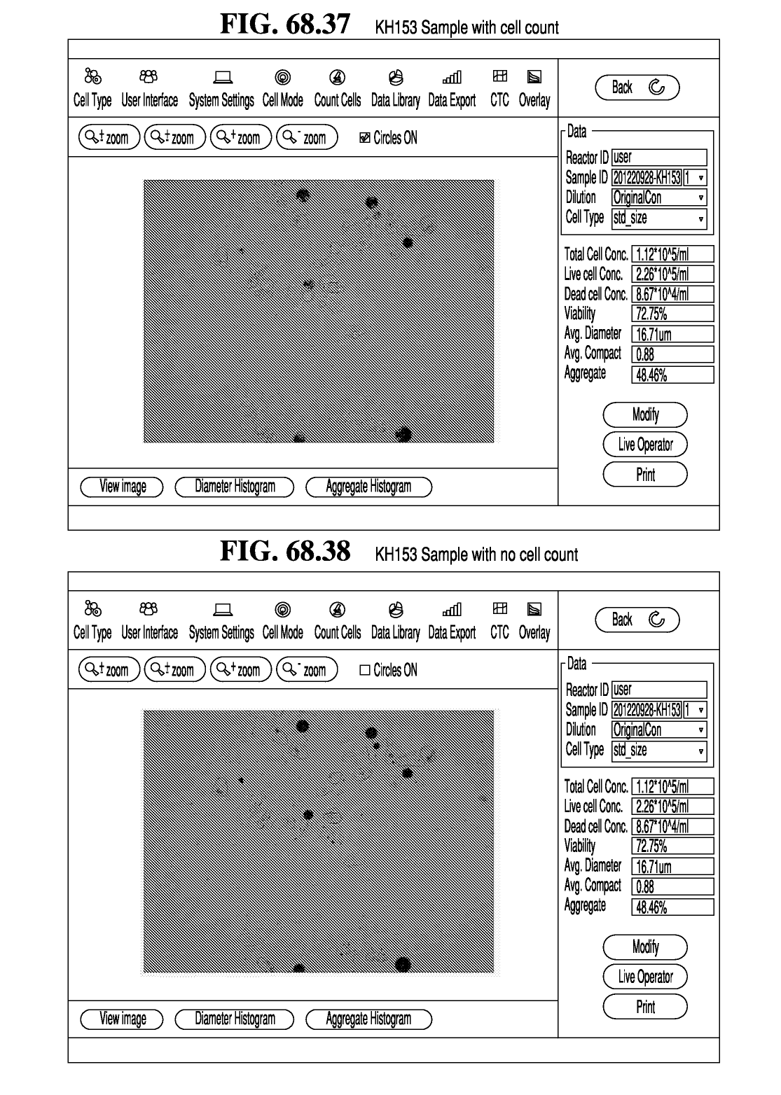

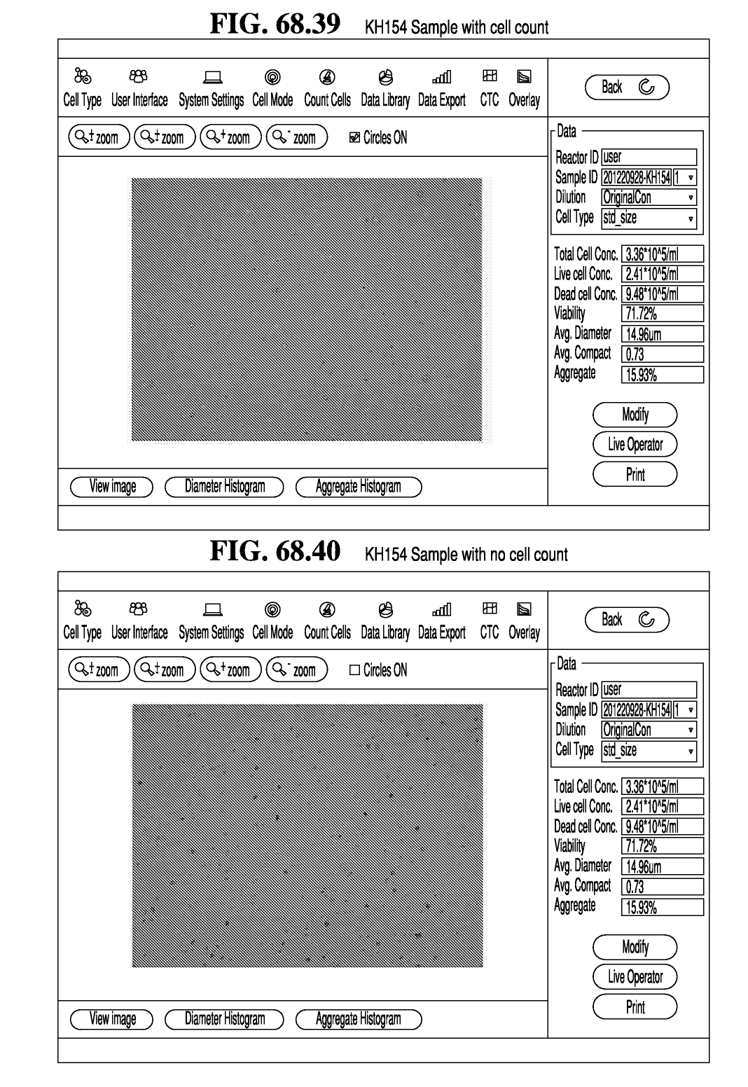

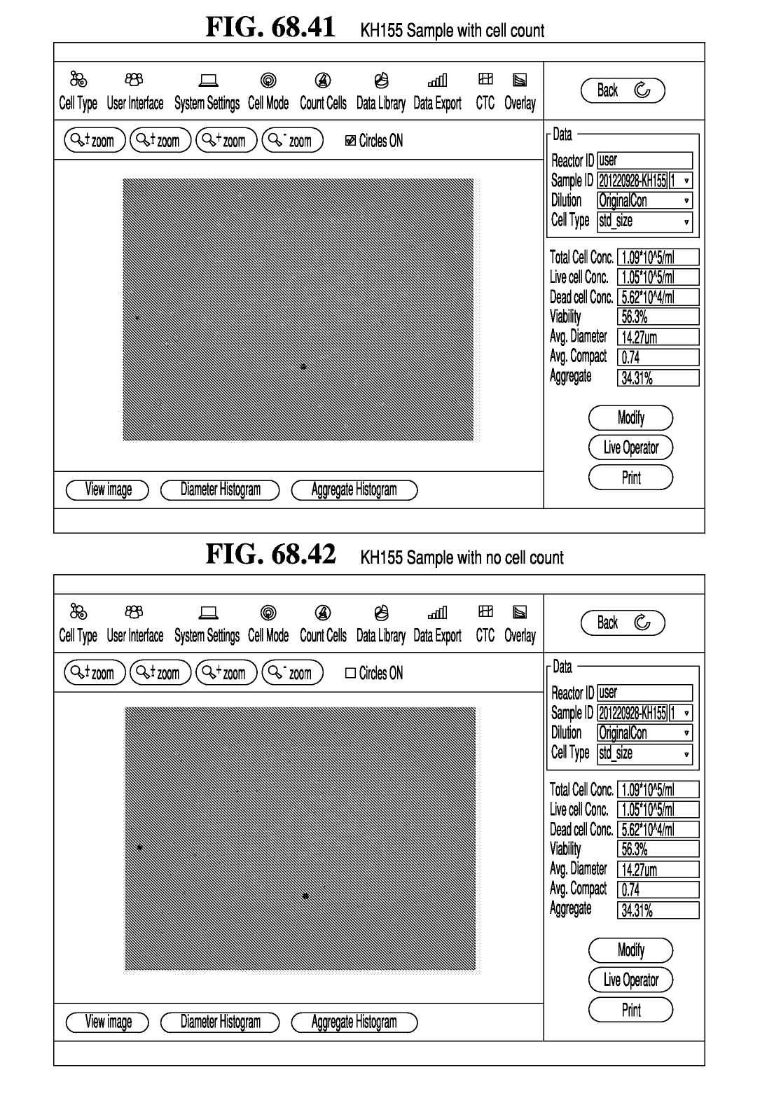

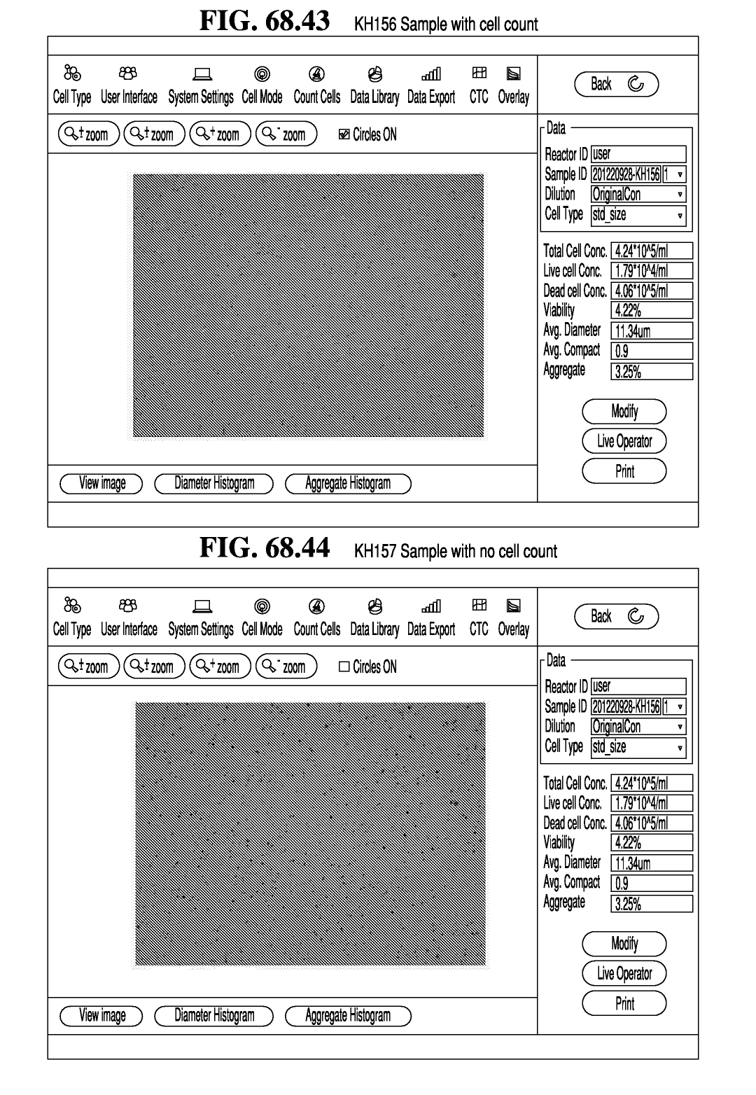

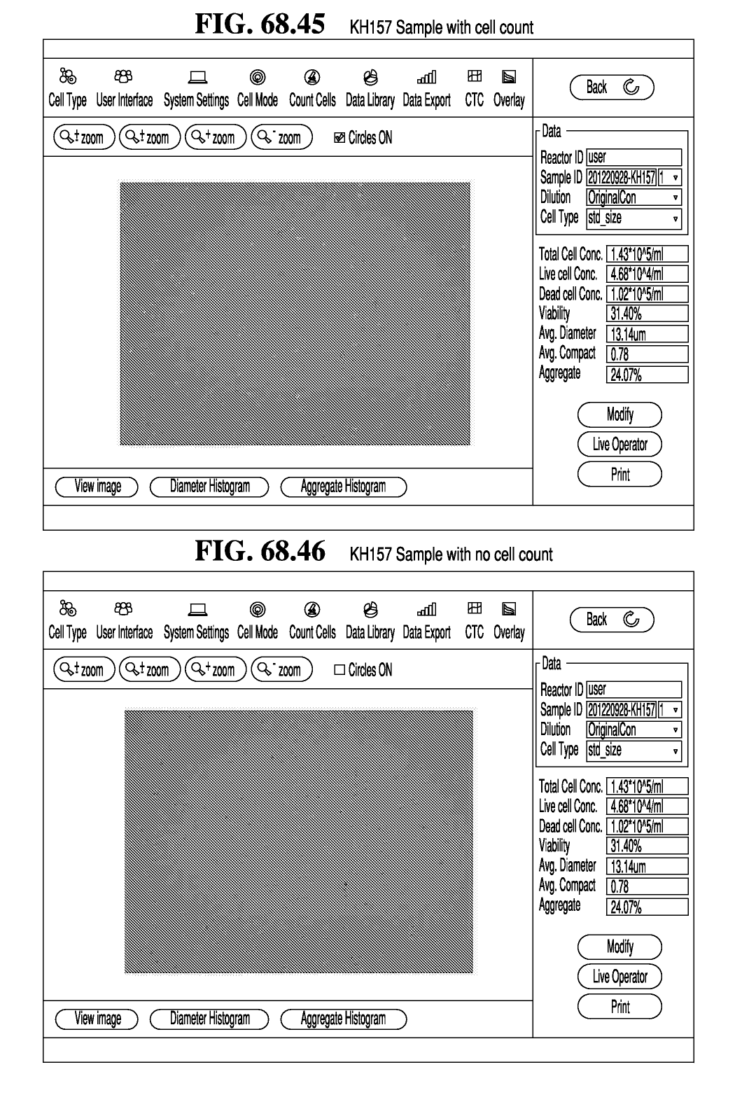

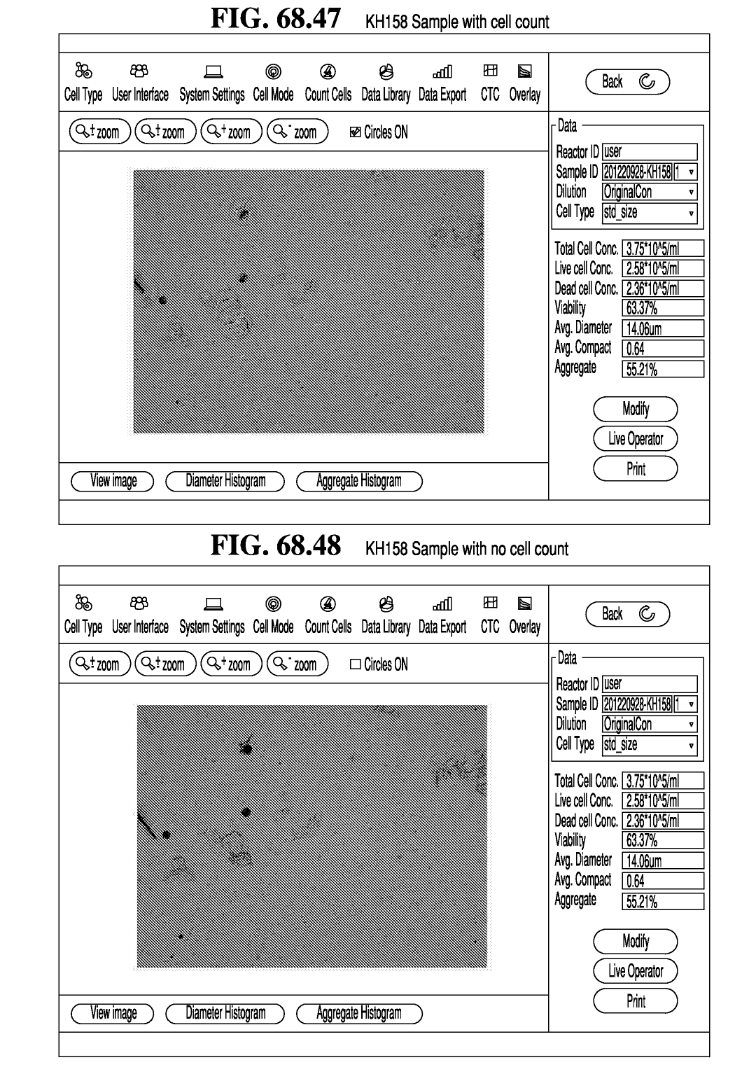

FIGS. 68.1 and 68.—KH136 consists of Big Banana

FIGS. 68.3 and 68.—KH137 consists of Small Banana

FIGS. 68.5 and 68.—KH138 consists of Star Fruit

FIGS. 68.7 and 68.—KH 139 consists of Pomegranate

FIGS. 68.9 and 68.1—KH 140 consists of Plum

FIGS. 68.11 and 68.1—KH141 consists of Mango

FIGS. 68.13 and 68.1—KH 142 consists of Green Hot Pepper

FIGS. 68.15 and 68.1—KH143 consists of Red Sweet Pepper

FIGS. 68.17 and 68.1—KH144 consists of Green Sweet Pepper

FIGS. 68.19 and 68.2—KH145 consists of Daisy Flower

FIGS. 68.21 and 68.2—KH146 consists of Puer Tea

FIGS. 68.23 and 68.2—KH147 consists of Walnut

FIGS. 68.25 and 68.2—KH148 consists of white bread

FIGS. 68.27 and 68.2—KH149 consists of Brown bread

FIGS. 68.29 and 68.3—KH150 consists of Garlic

FIGS. 68.31 and 68.3—KH151 consists of Ginger

FIGS. 68.33 and 68.3—KH152 consists of Persimmon

FIGS. 68.35 and 68.3—KH153 consists of Papaya

FIGS. 68.37 and 68.3—KH154 consists of Broccoli

FIGS. 68.39 and 68.4—KH155 consists of Onion

FIGS. 68.41 and 68.4—KH156 consists of Pumpkin

FIGS. 68.43 and 68.4—KH157 consists of Wax Gourd

FIGS. 68.45 and 68.4—KH158 consists of Towel Gourd

FIGS. 68.47 and 68.4

KH201 through KH214 mediums are all meat based.
KH 201 medium contains 18.8 g of paste of Green Mussel with 380 mL of WFI. Sample number 1, Tryptophan is added as a stabilizer.

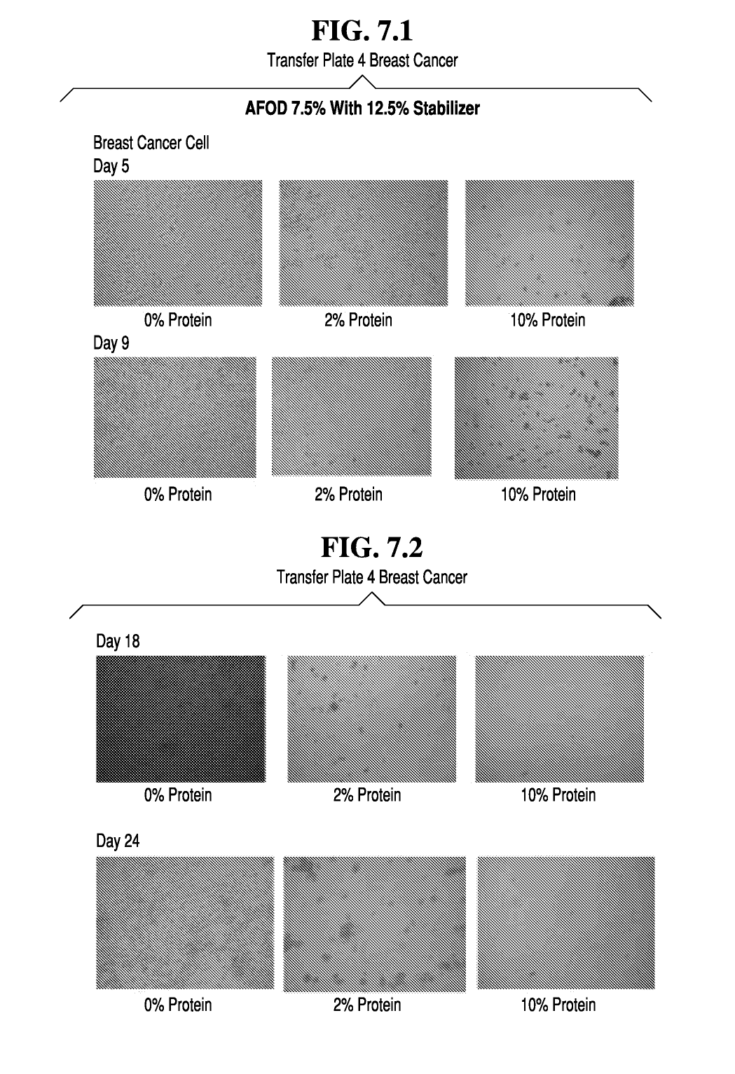

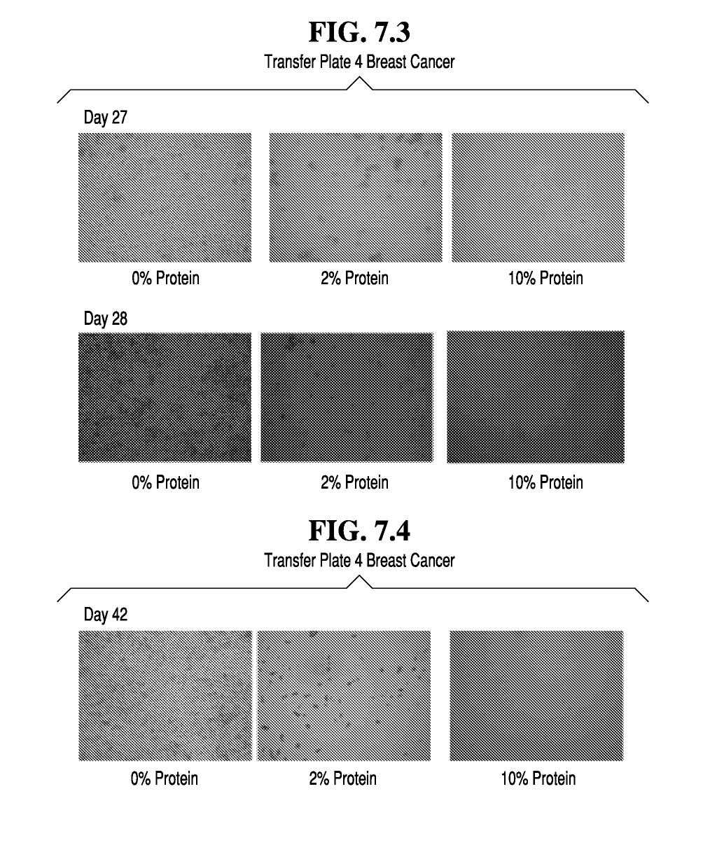

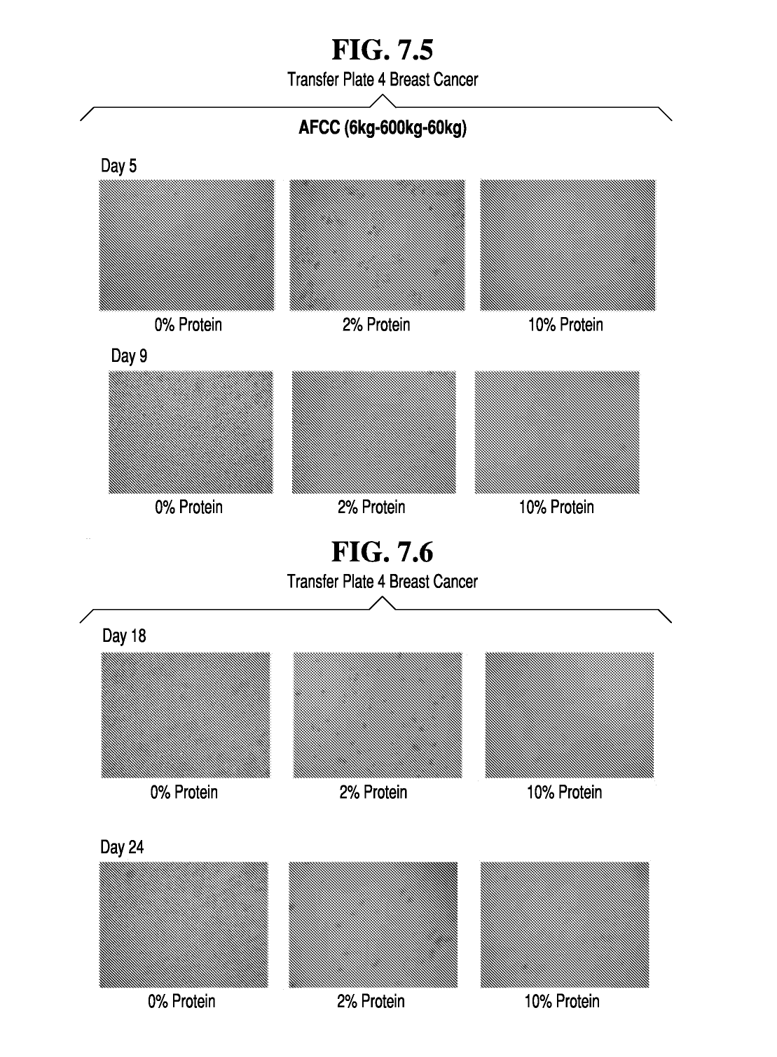

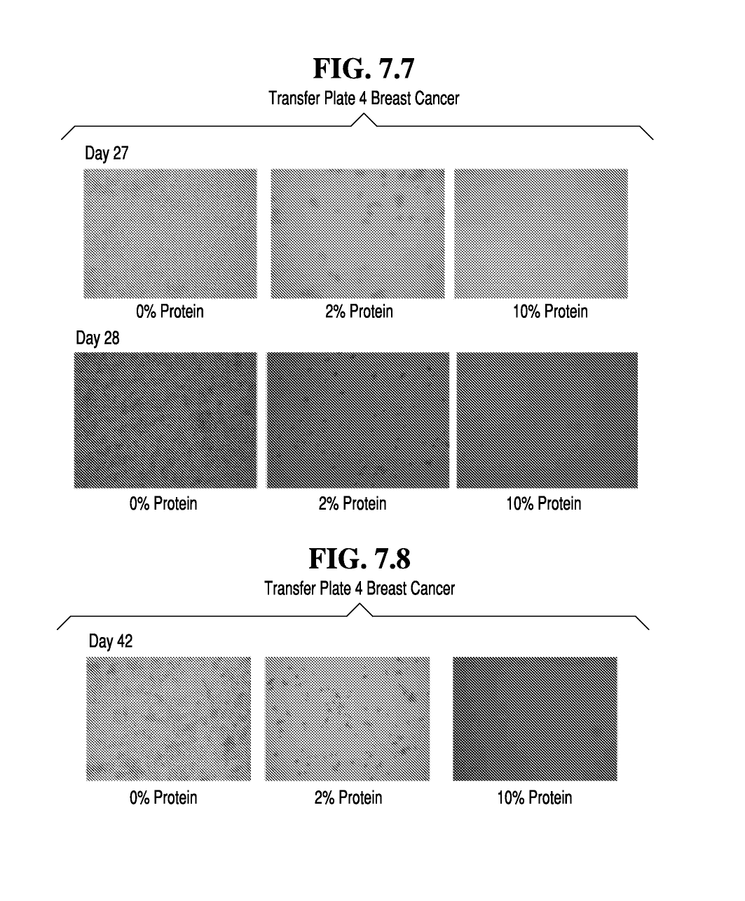

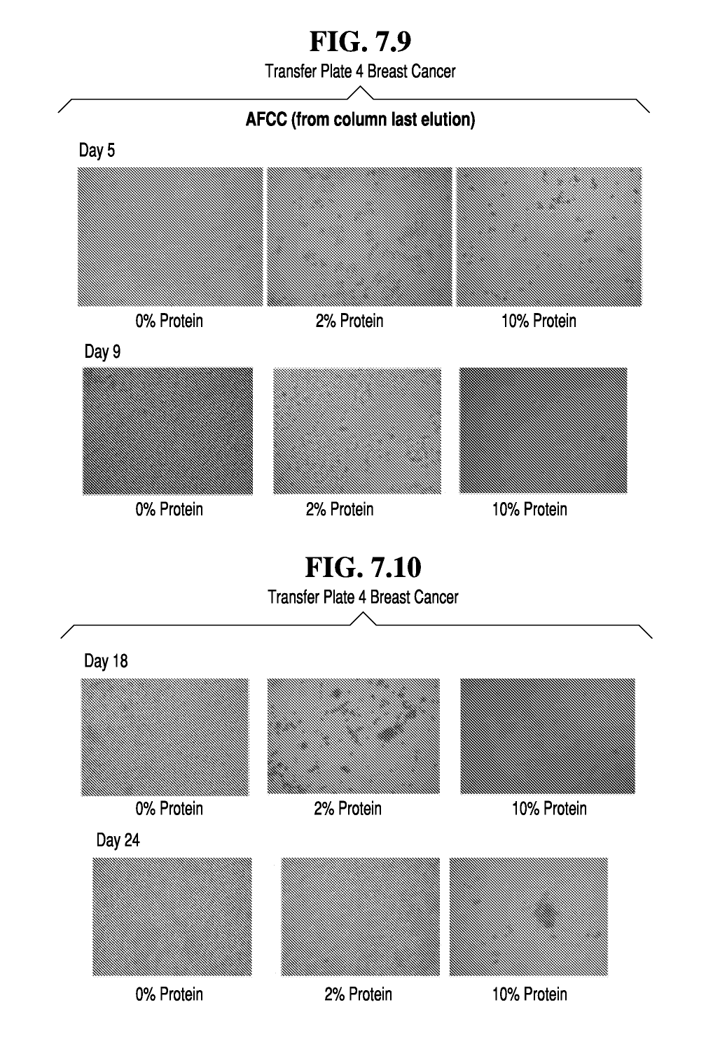

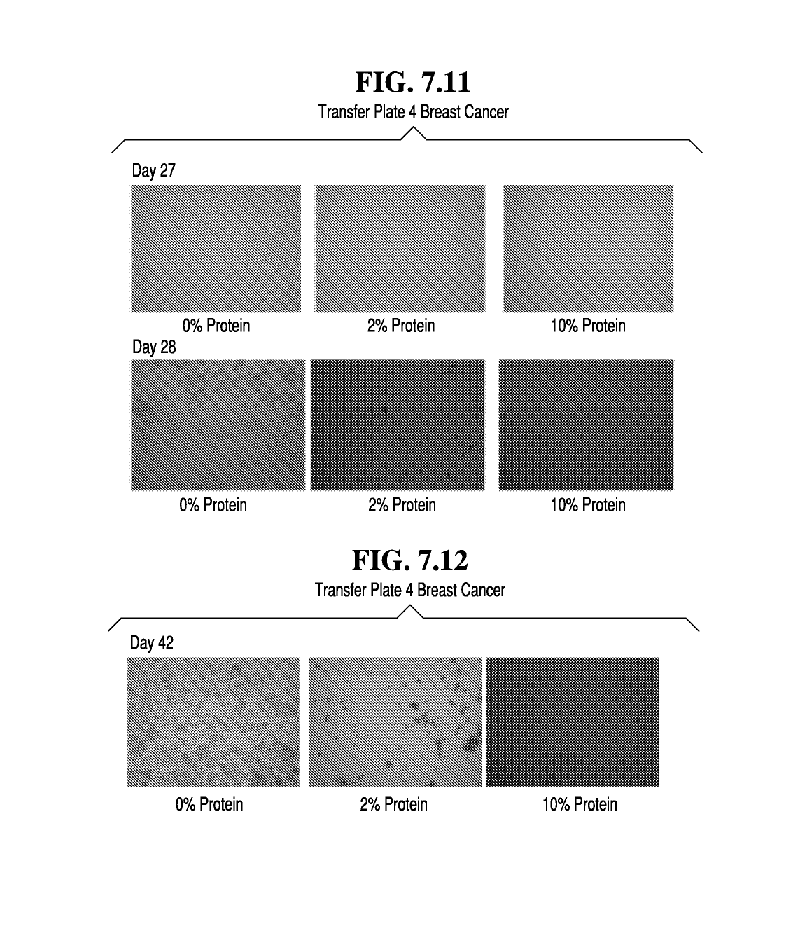

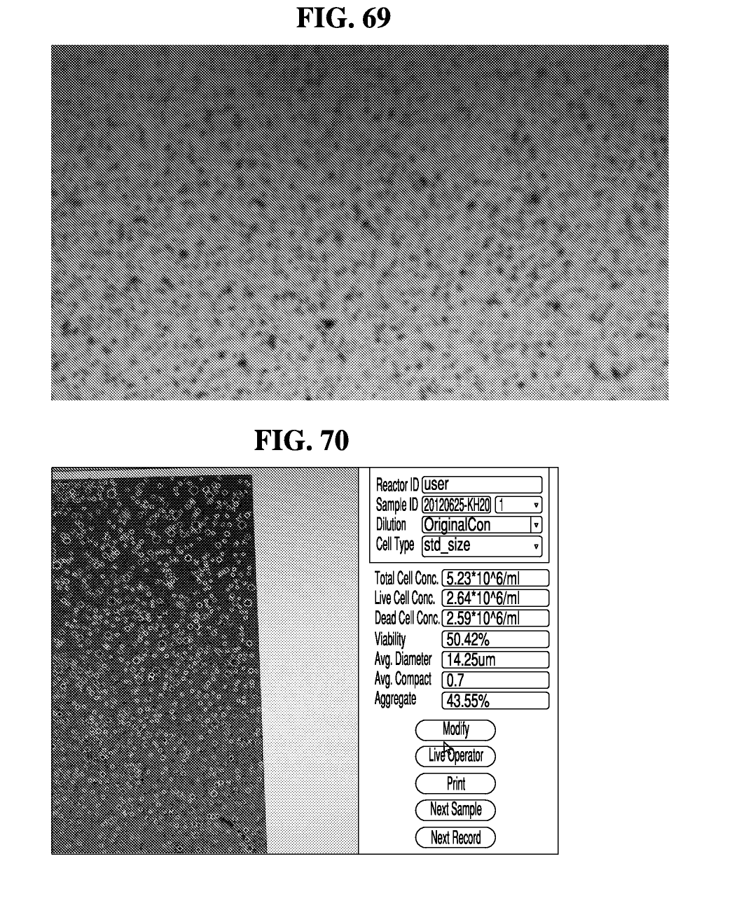

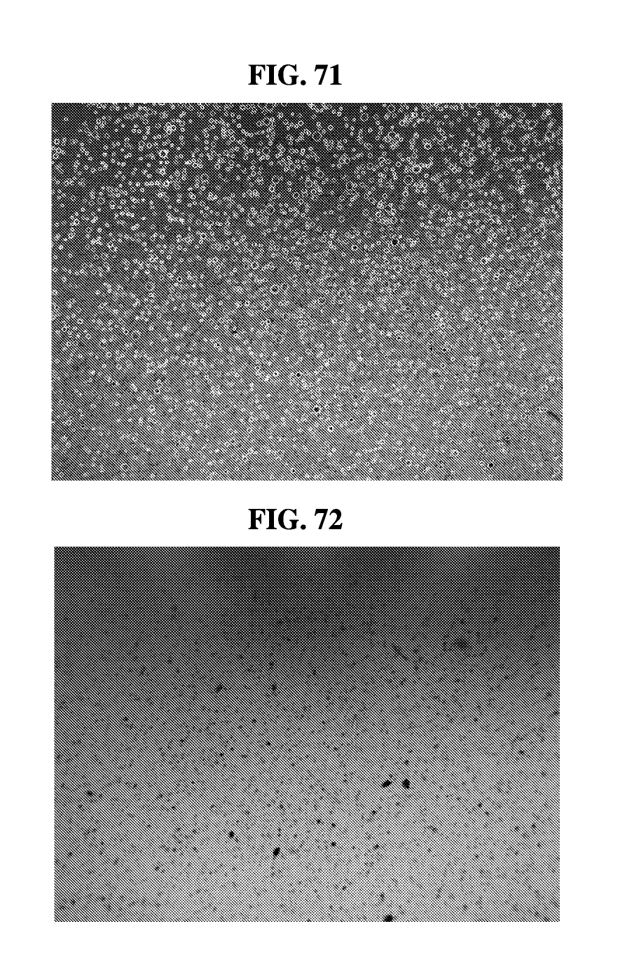

FIGS. 69, 70 and 7—KH201 medium sample number 2. Tryptophan is added as a stabilizer.

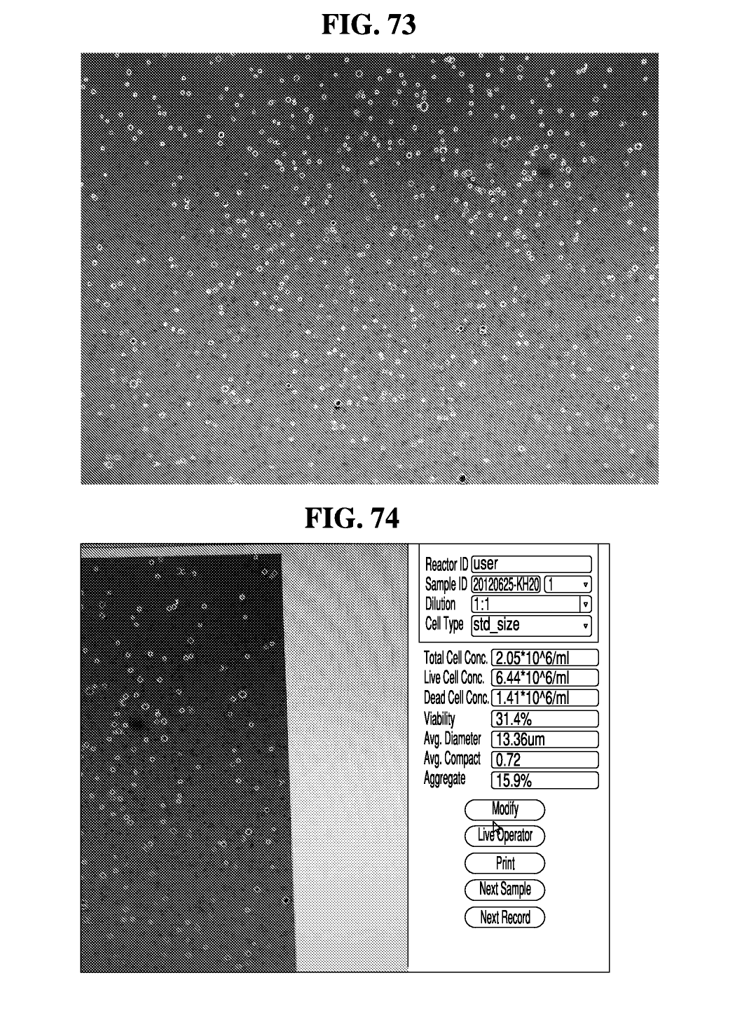

FIGS. 72, 73 and 7—KH201 medium sample number 3. Tryptophan is added as a stabilizer.

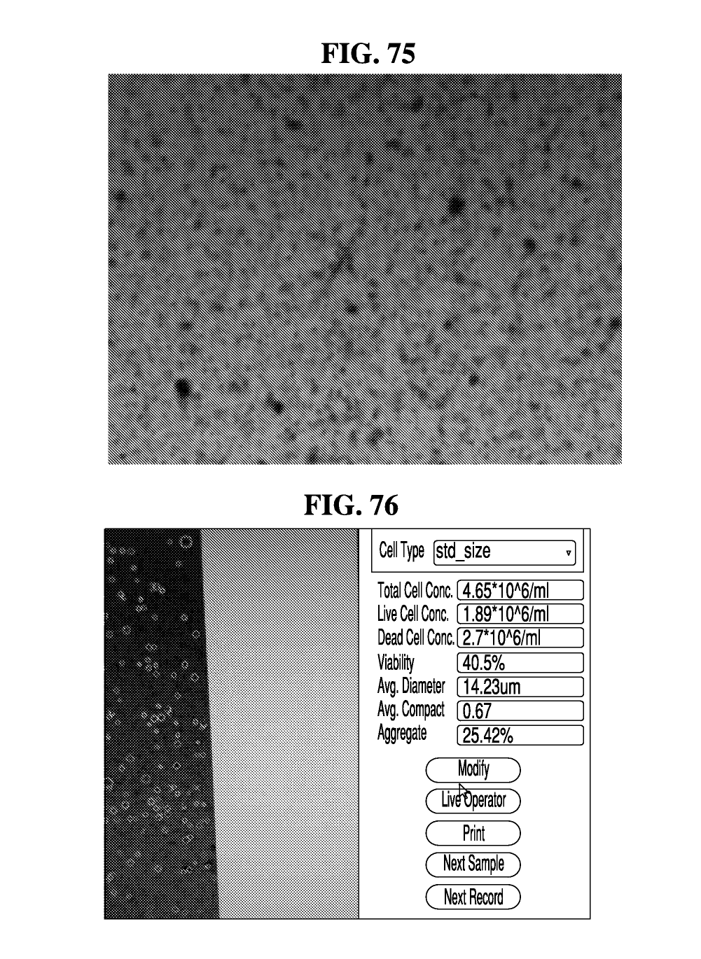

FIGS. 75, 76 and 7—KH201 medium sample number 4. Without Tryptophan.

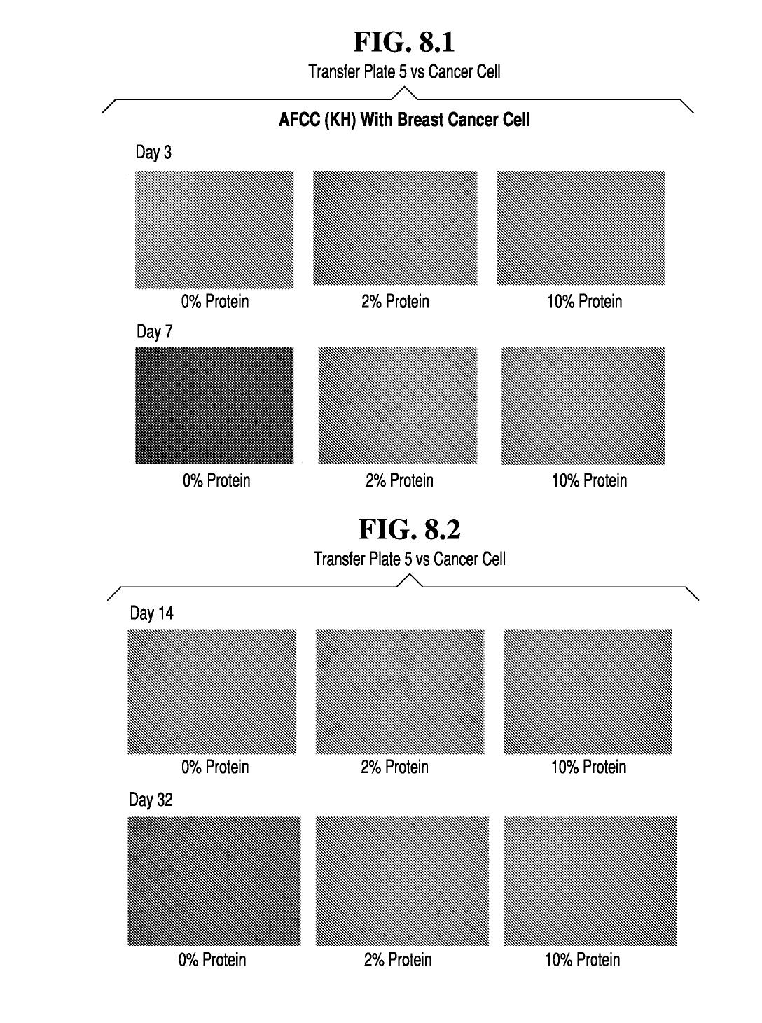

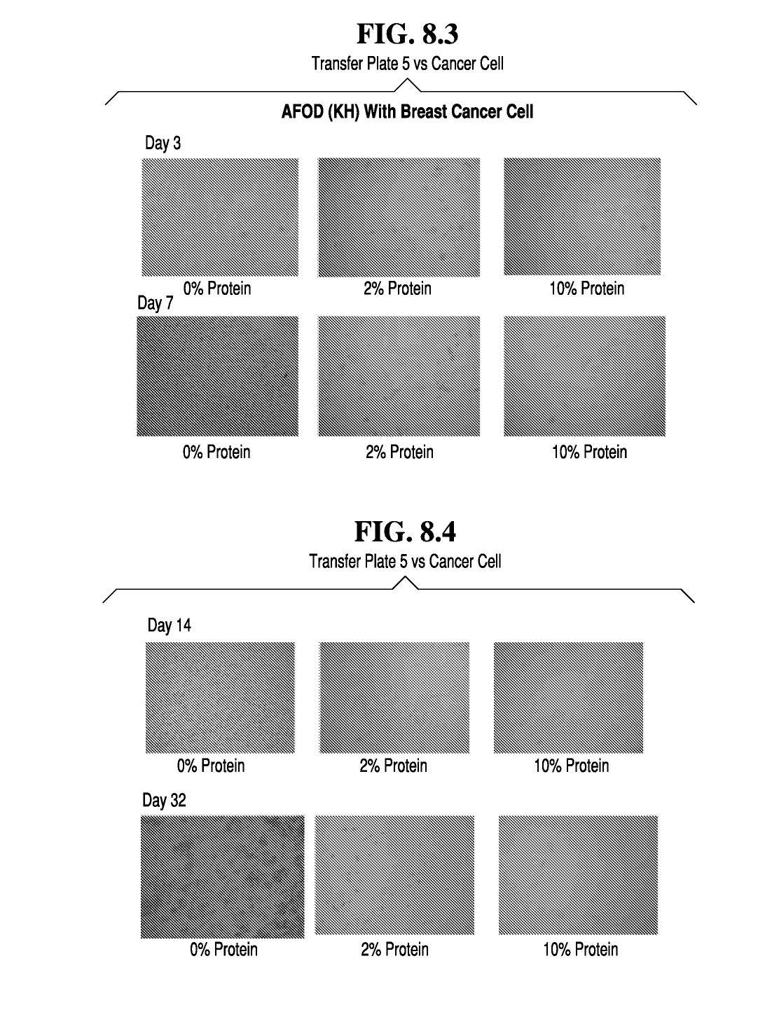

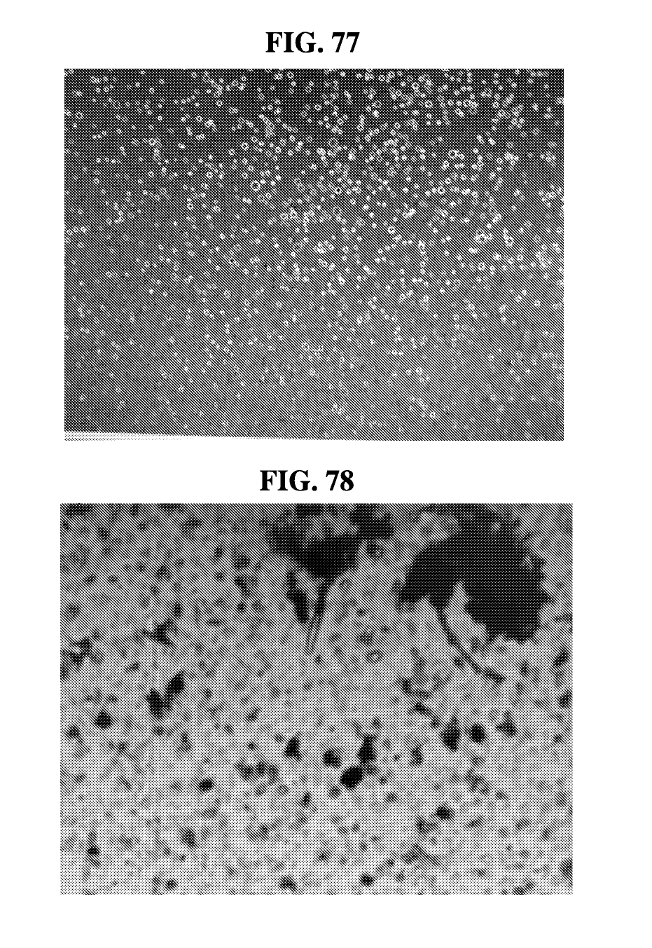

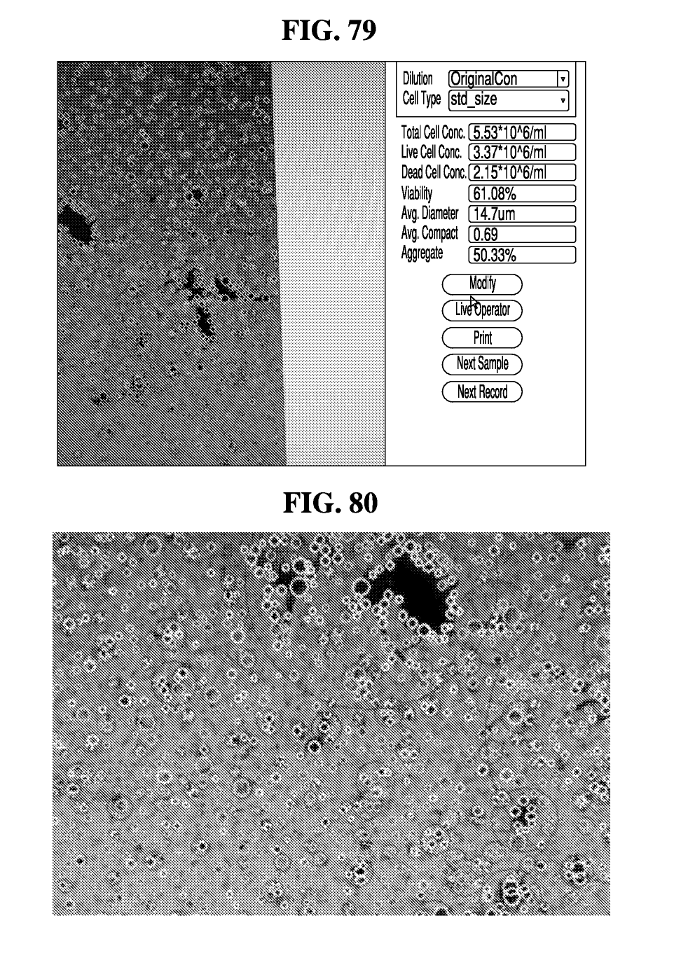

FIGS. 78, 79 and 8—KH201 medium sample number 5. Without Tryptophan.

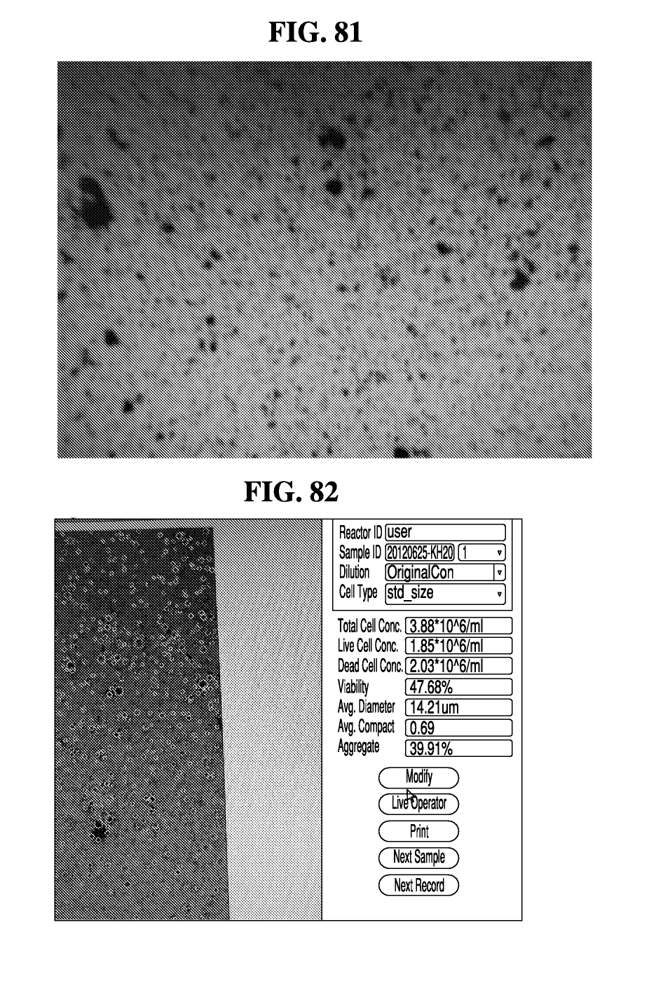

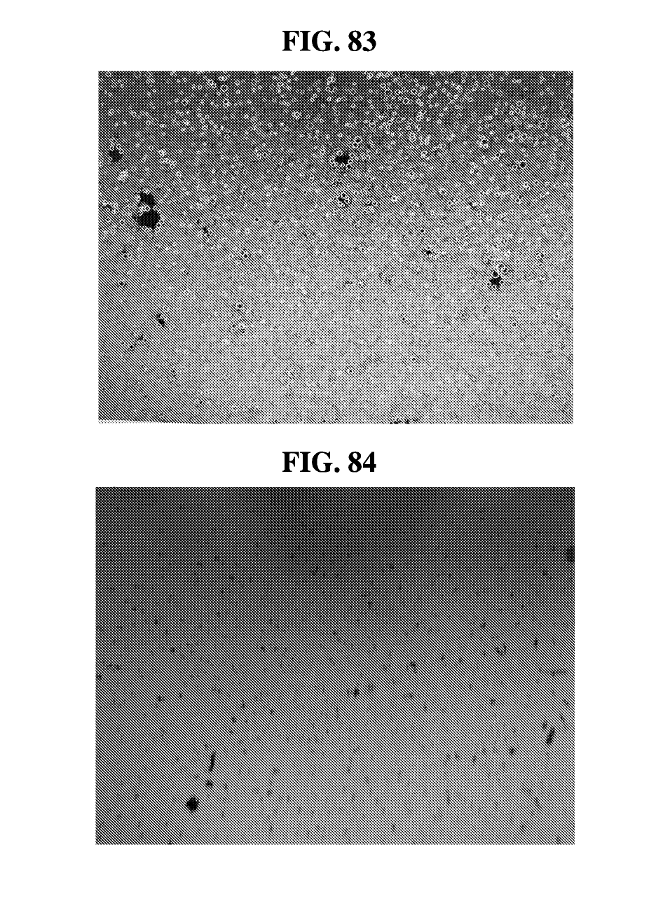

FIGS. 81, 82 and 8—KH 202 medium contains 42 g of paste of duck with 800 mL of WFI. Sample number 1, Tryptophan is added as a stabilizer.

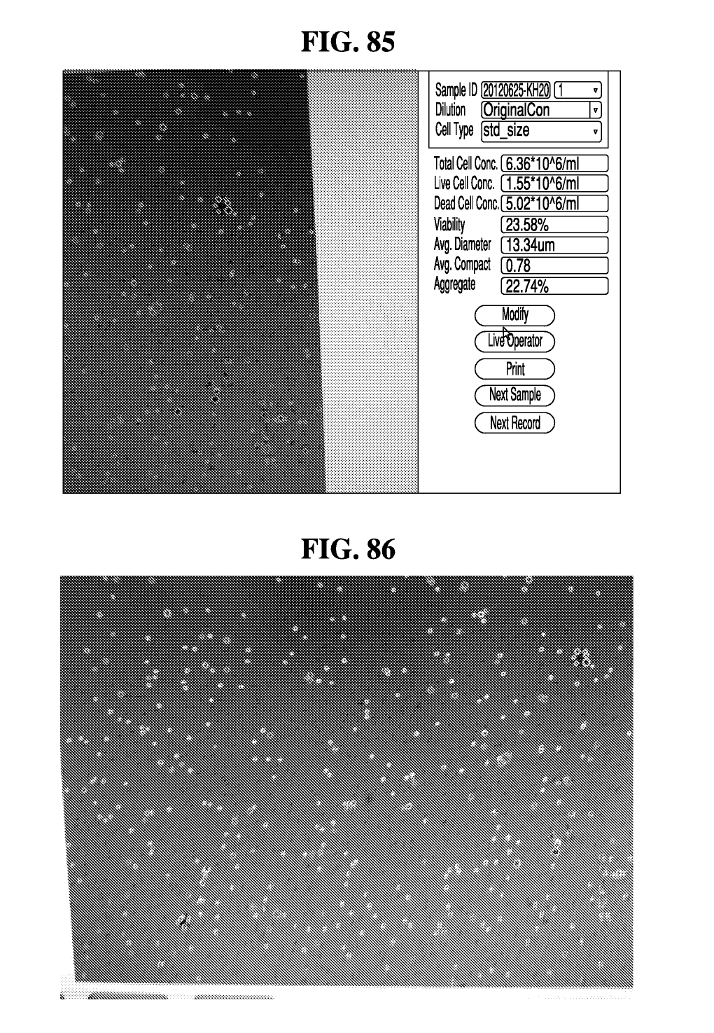

FIGS. 84, 85 and 8—KH202 medium sample number 2. Tryptophan is added as a stabilizer.

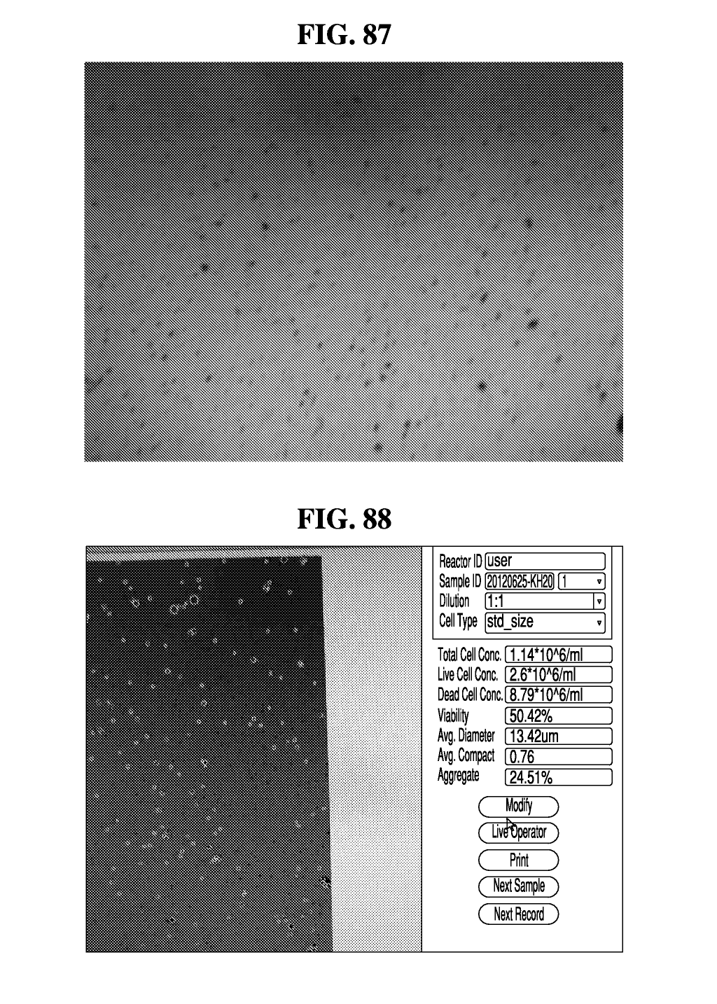

FIGS. 87, 88 and 8—KH202 medium sample number 3. Tryptophan is added as a stabilizer.

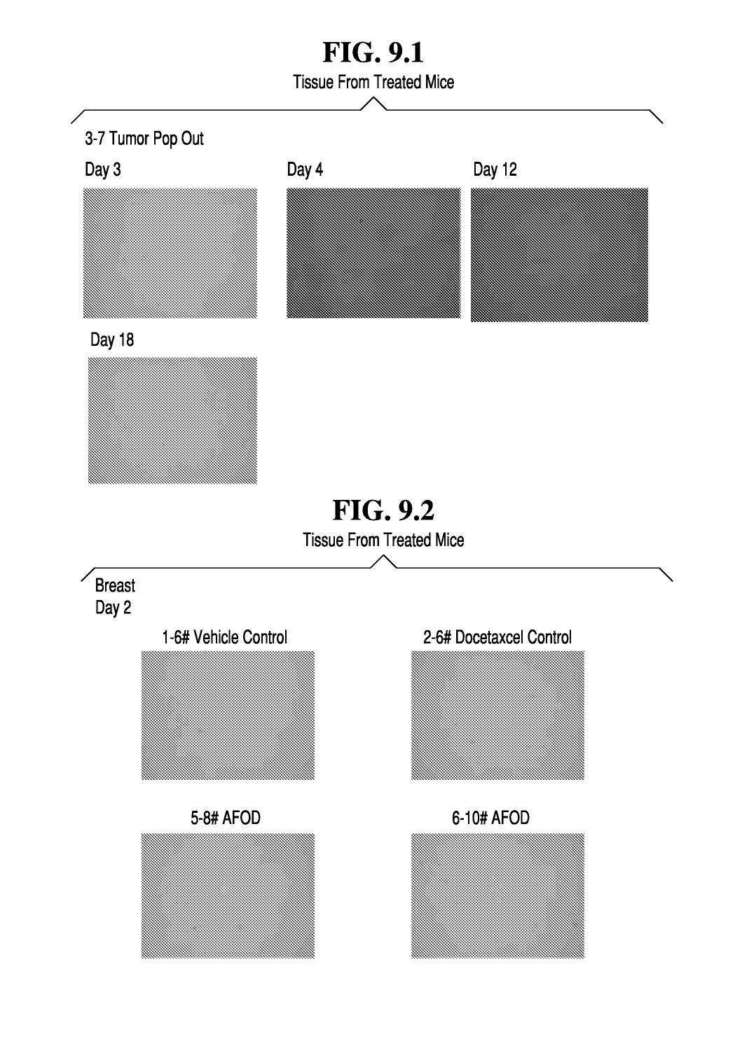

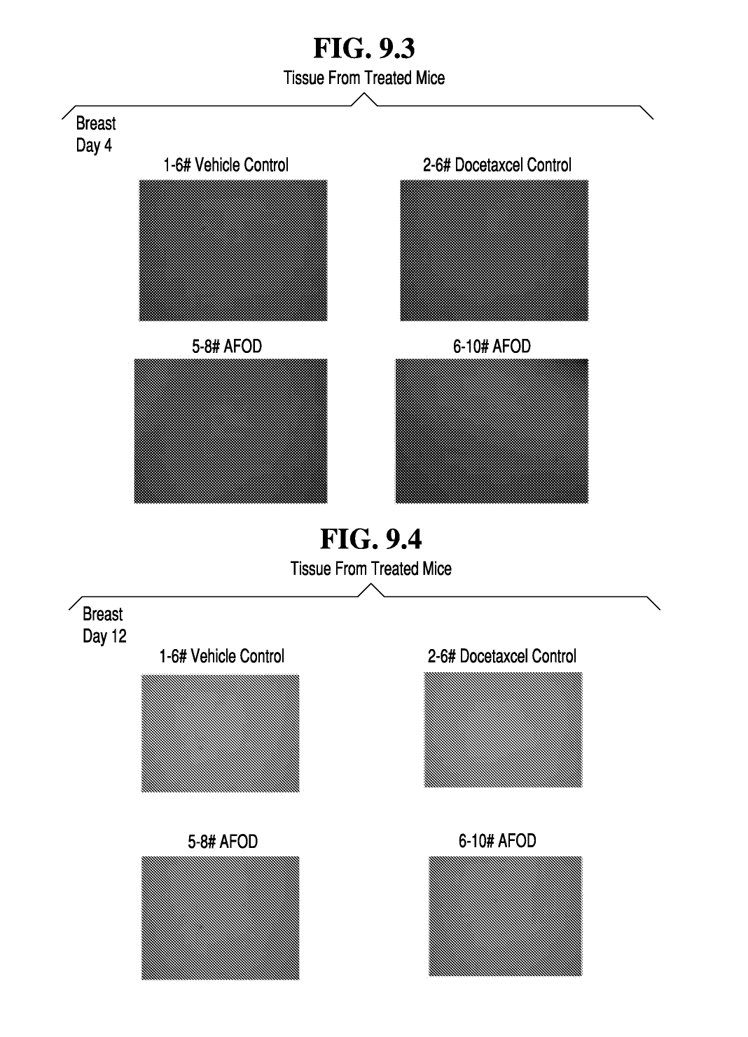

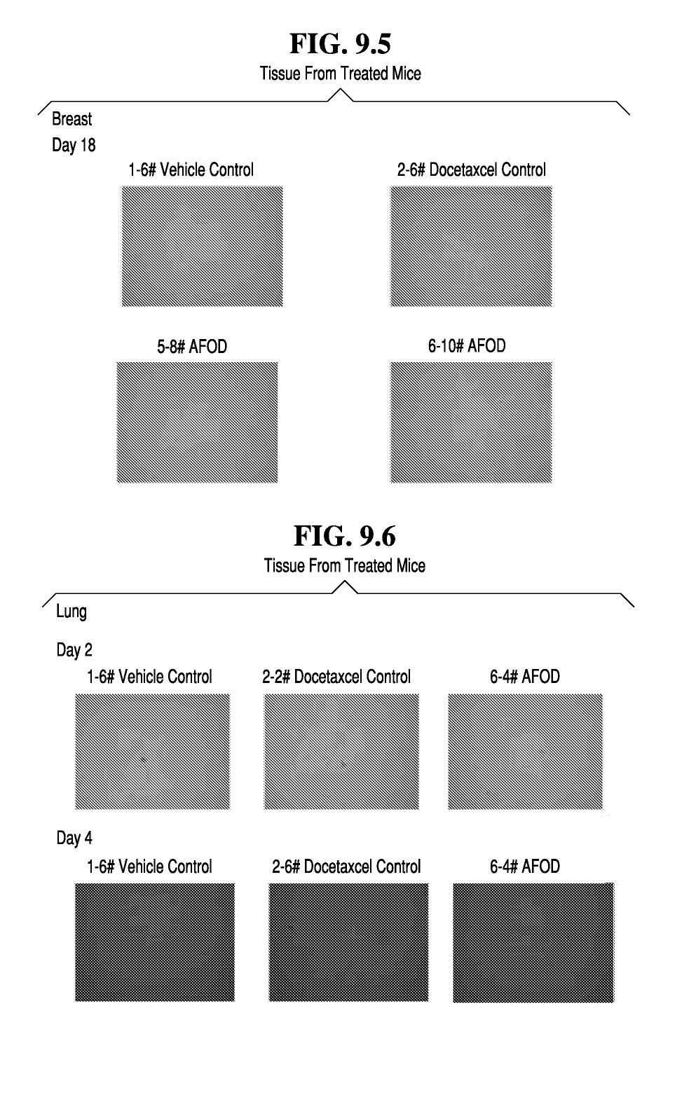

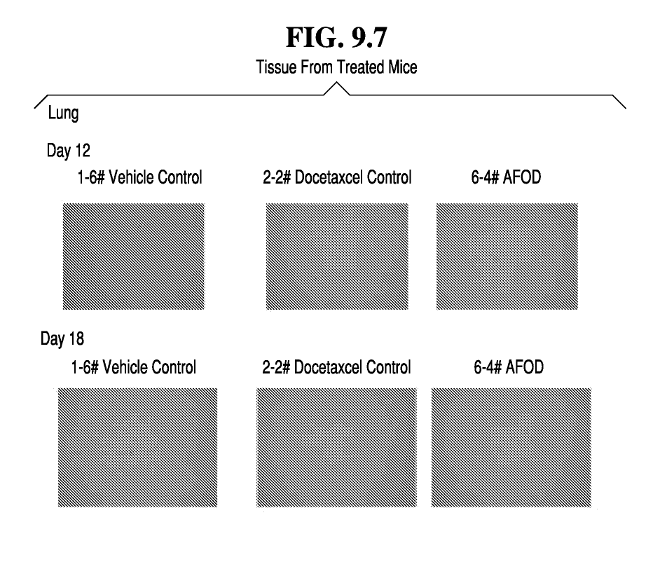

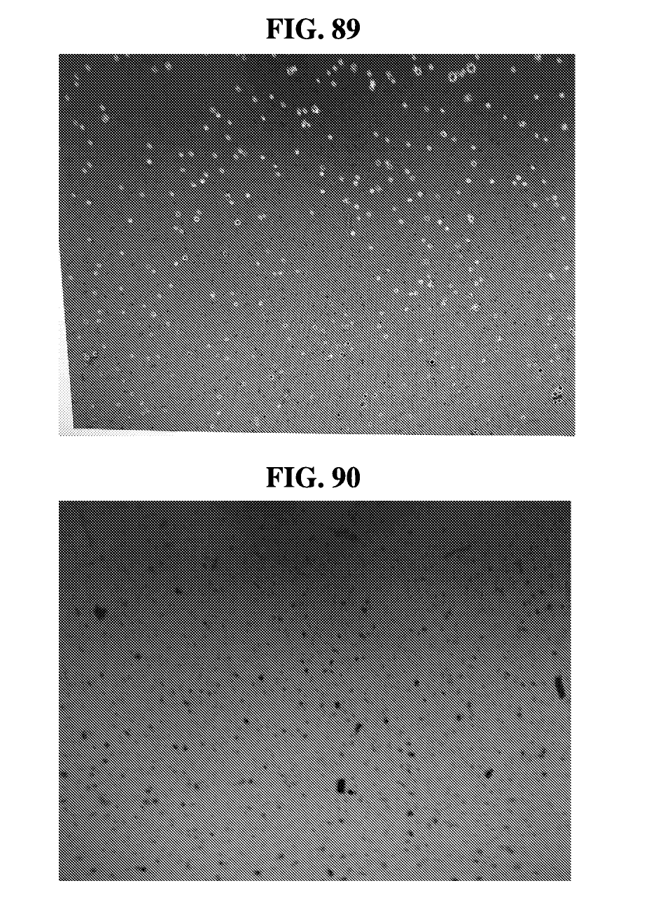

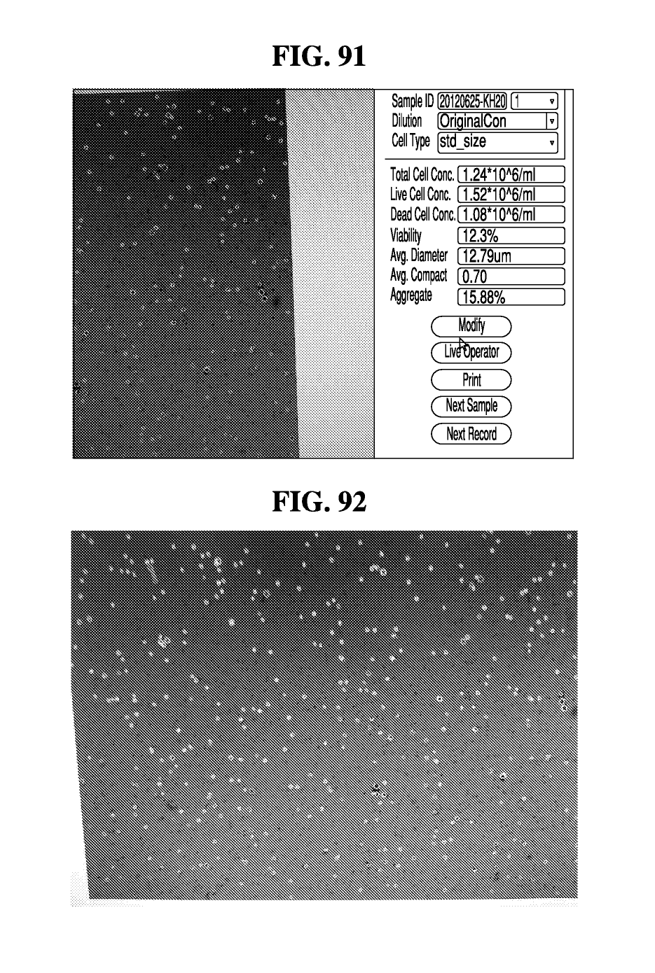

FIGS. 90, 91 and 9—KH202 medium sample number 4. Without Tryptophan.

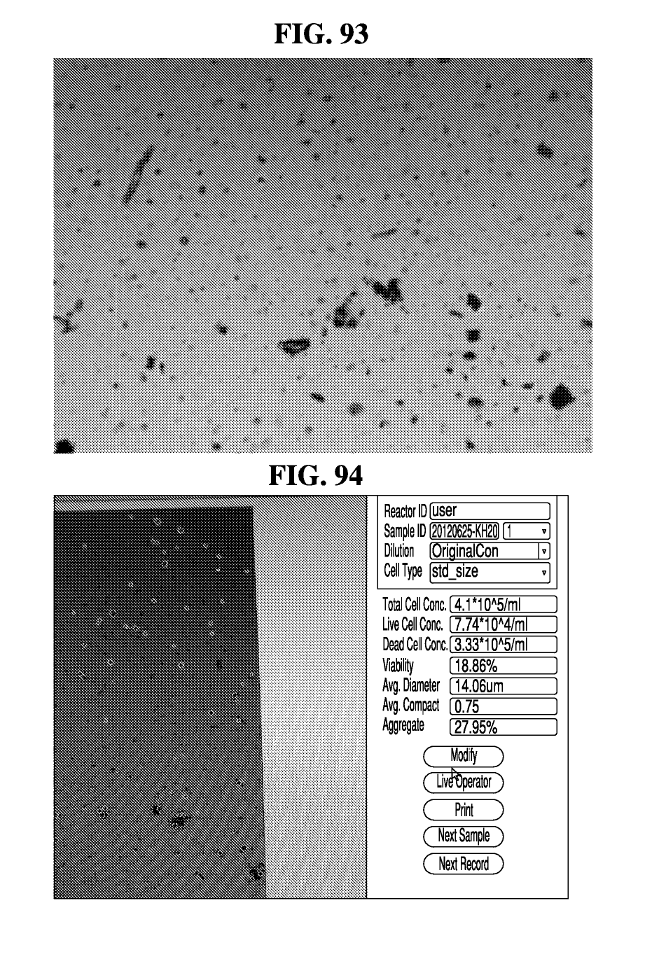

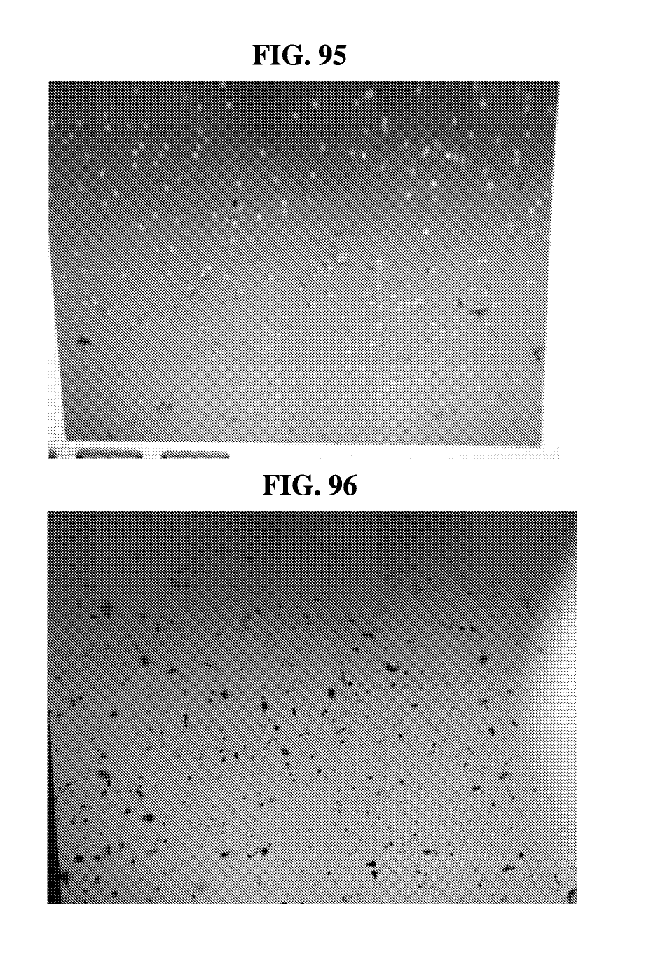

FIGS. 93, 94 and 9—KH202 medium sample number 5. Without Tryptophan.

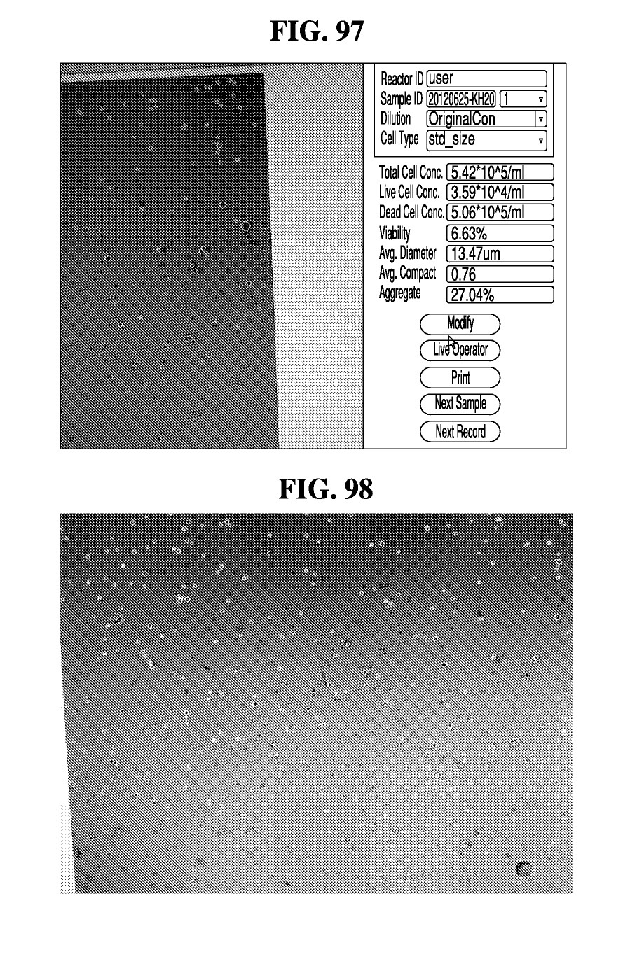

FIGS. 96, 97 and 9—KH 203 medium contains 40 g of paste of Giant Clam with 800 mL of WFI. Sample number 1, Tryptophan is added as a stabilizer.

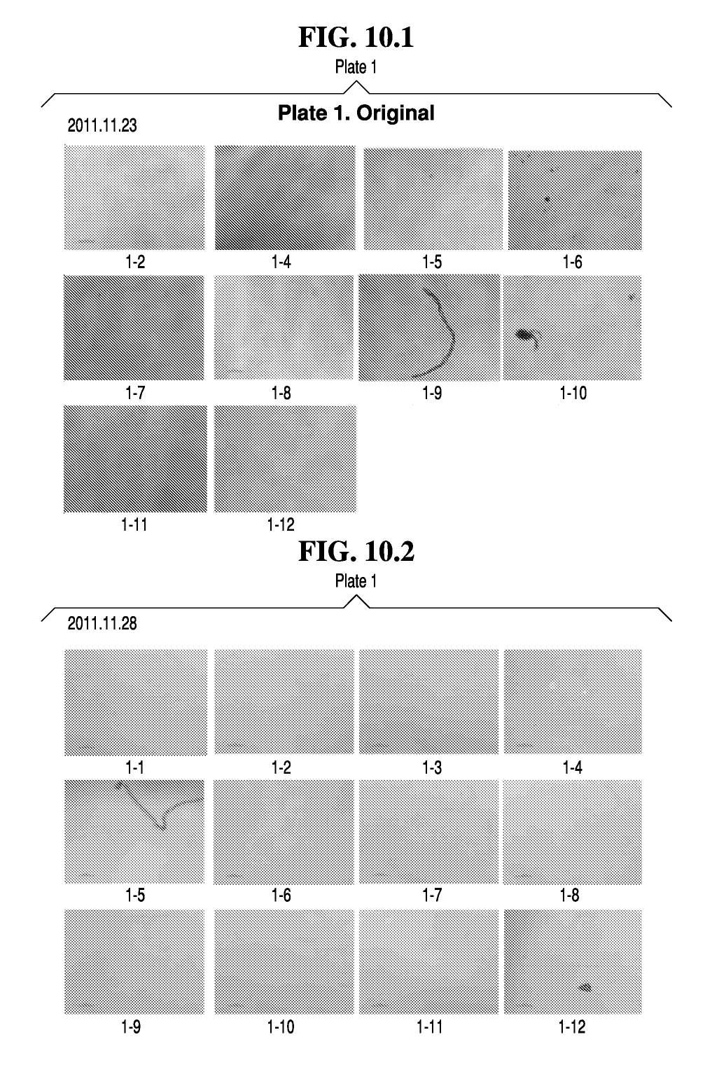

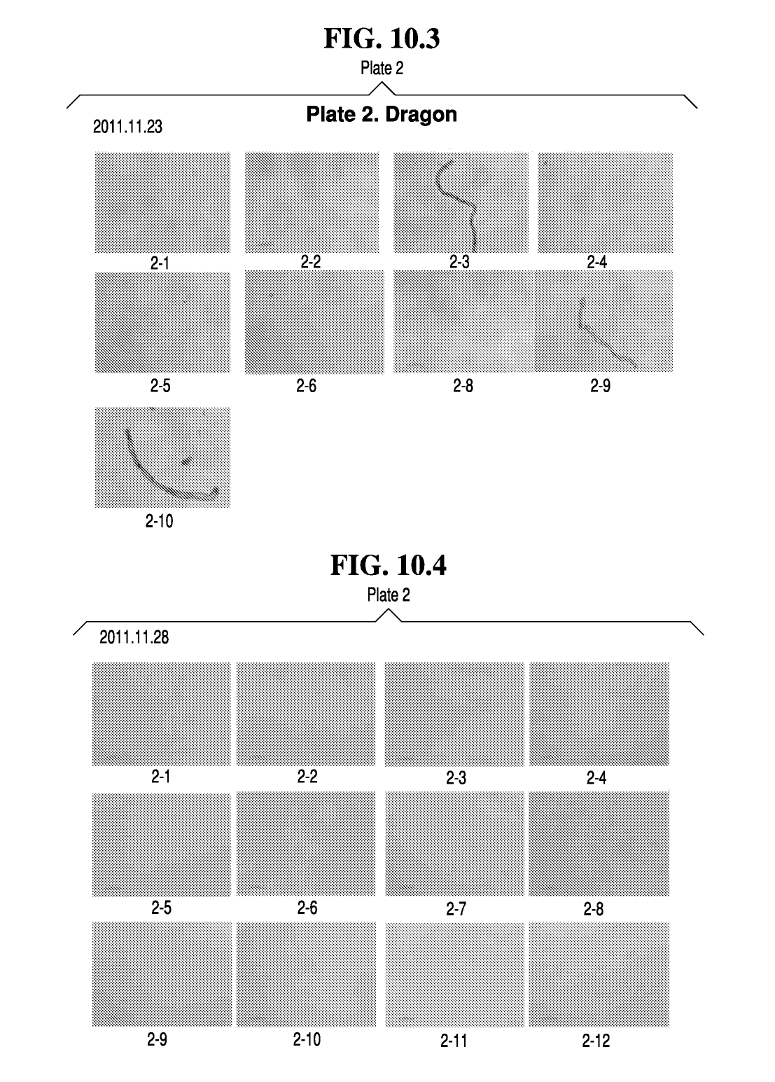

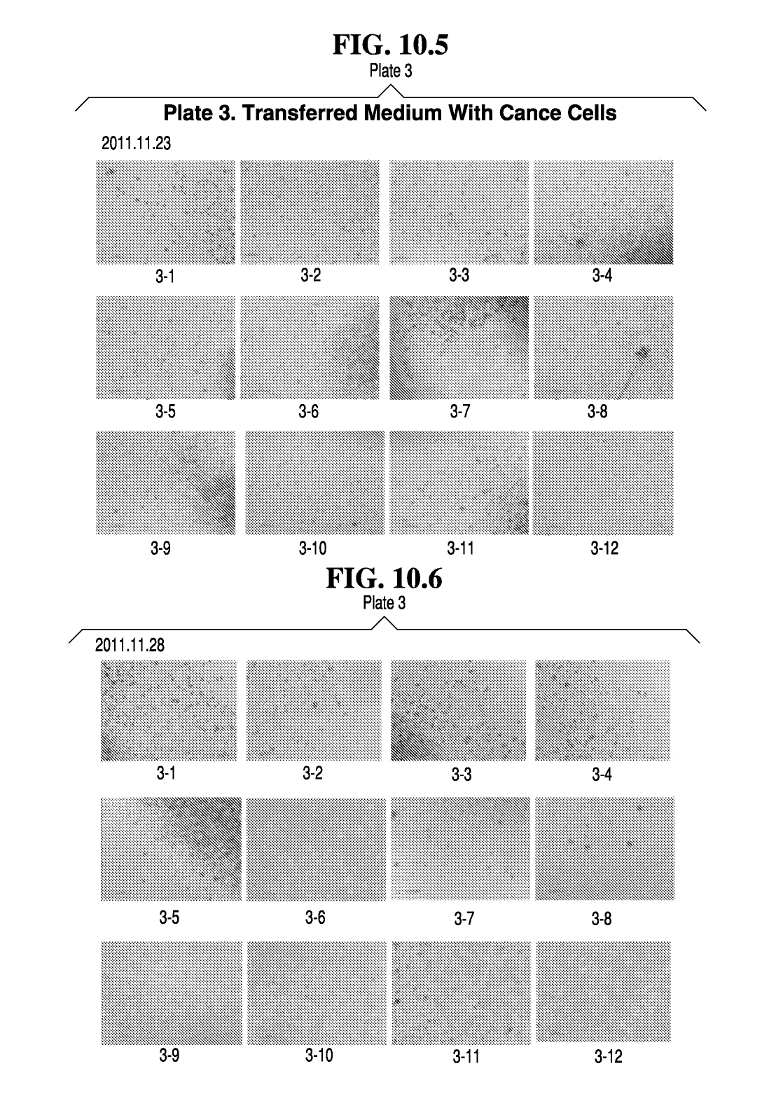

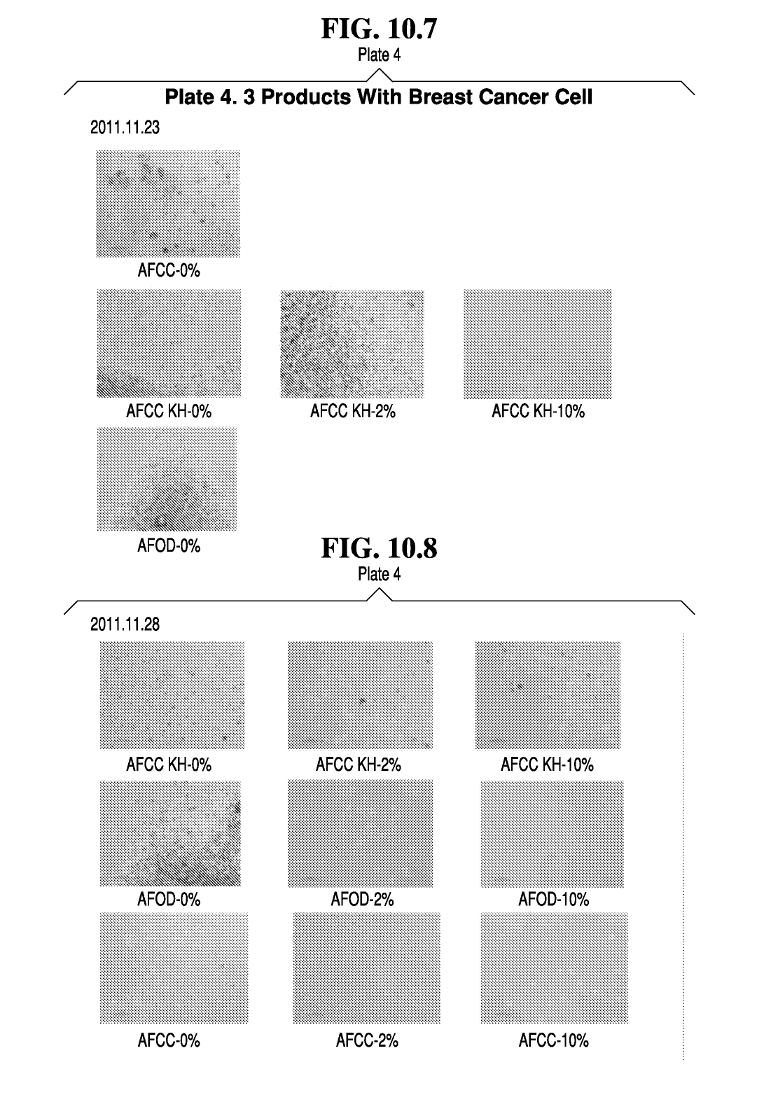

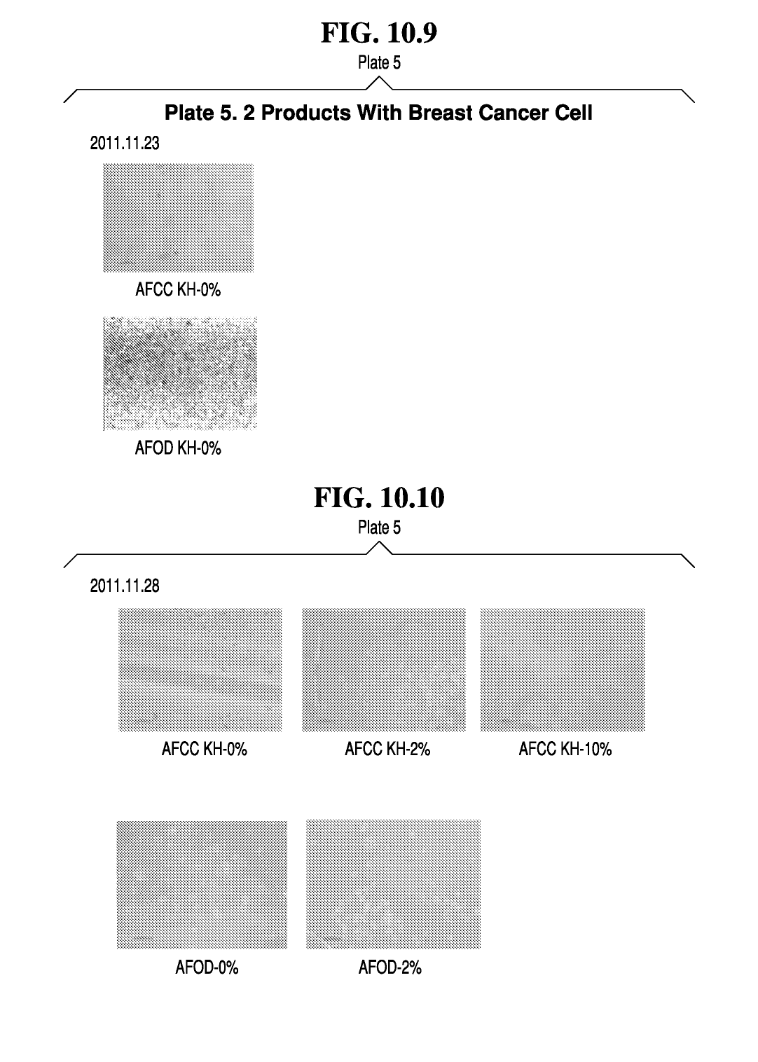

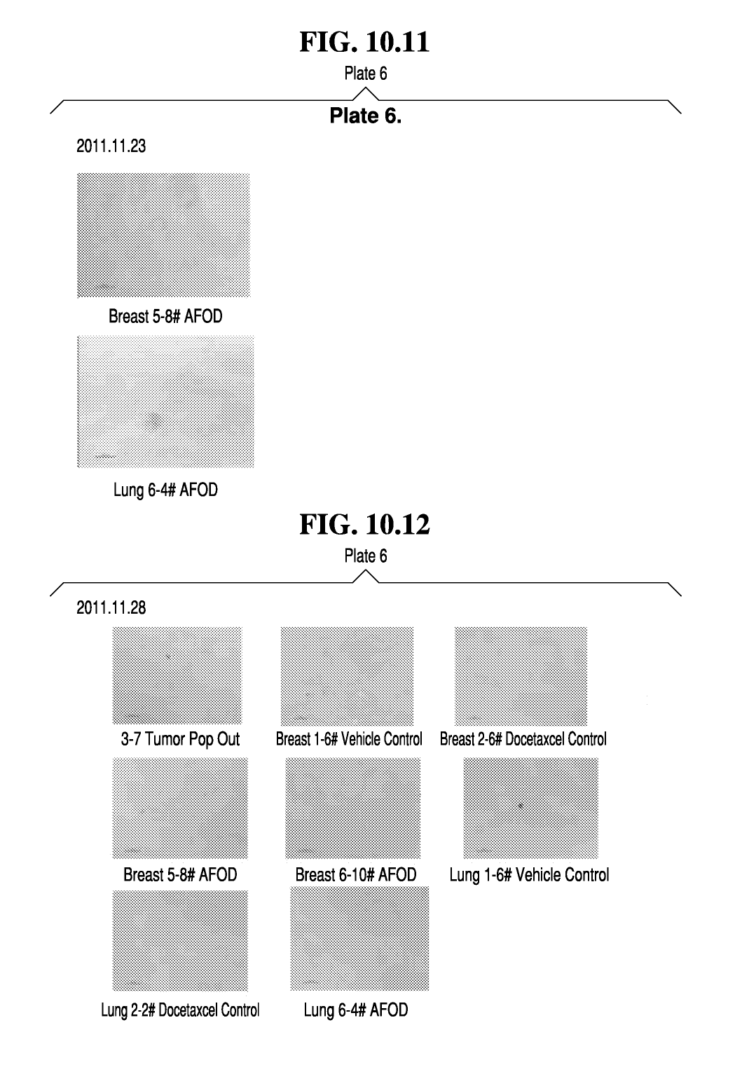

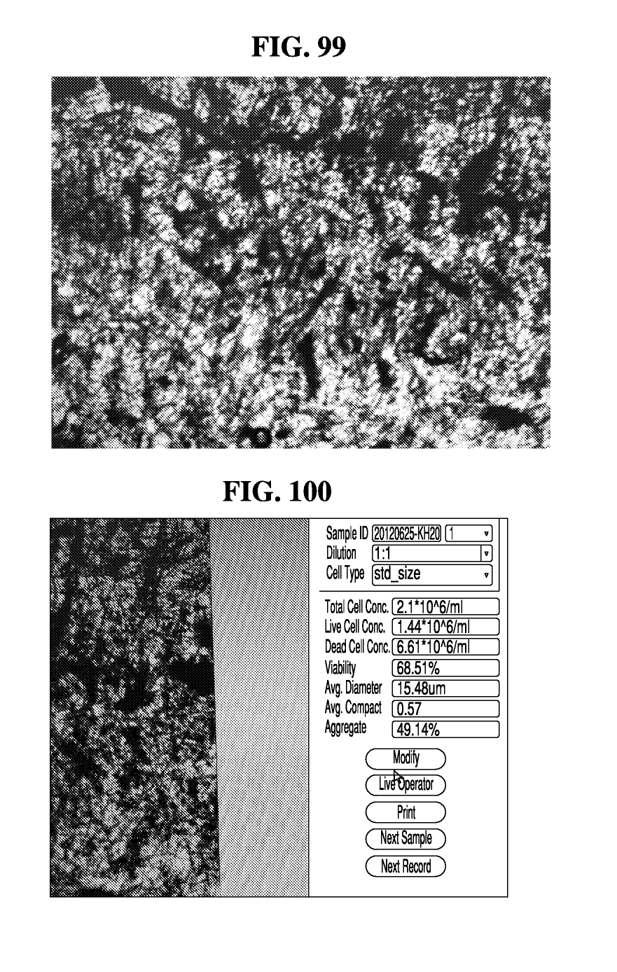

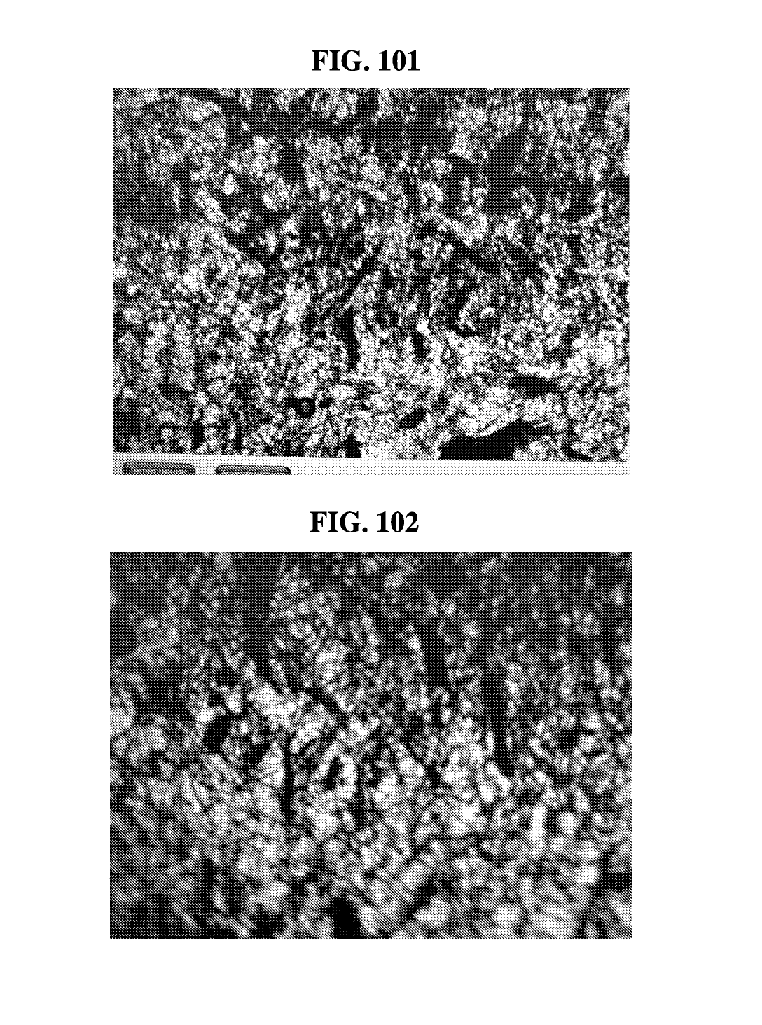

FIGS. 99, 100 and 10—KH203 medium sample number 2. Tryptophan is added as a stabilizer.

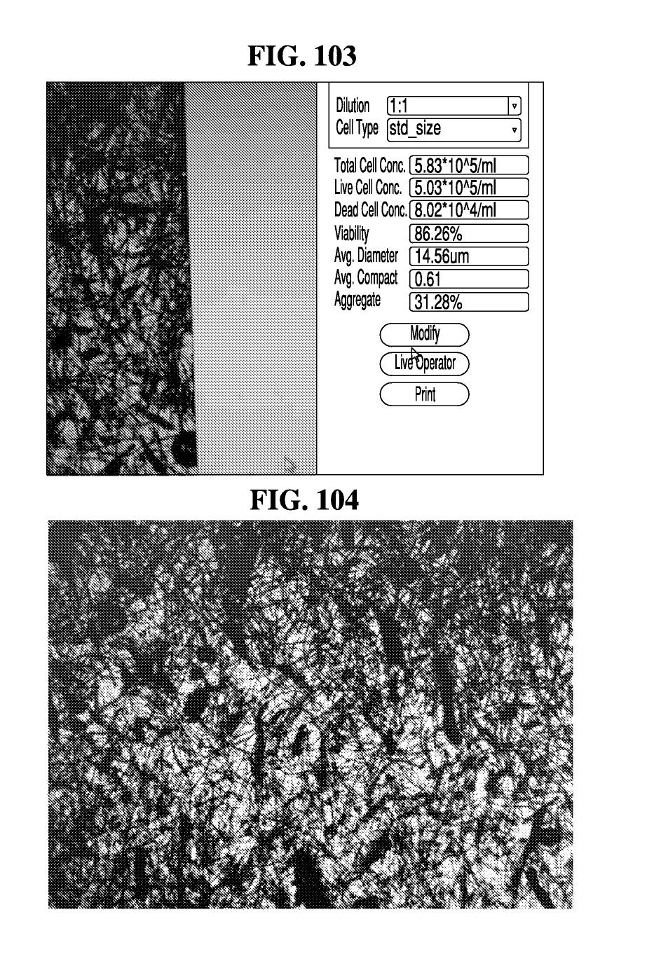

FIGS. 102, 103 and 10—KH203 medium sample number 3. Tryptophan is added as a stabilizer.

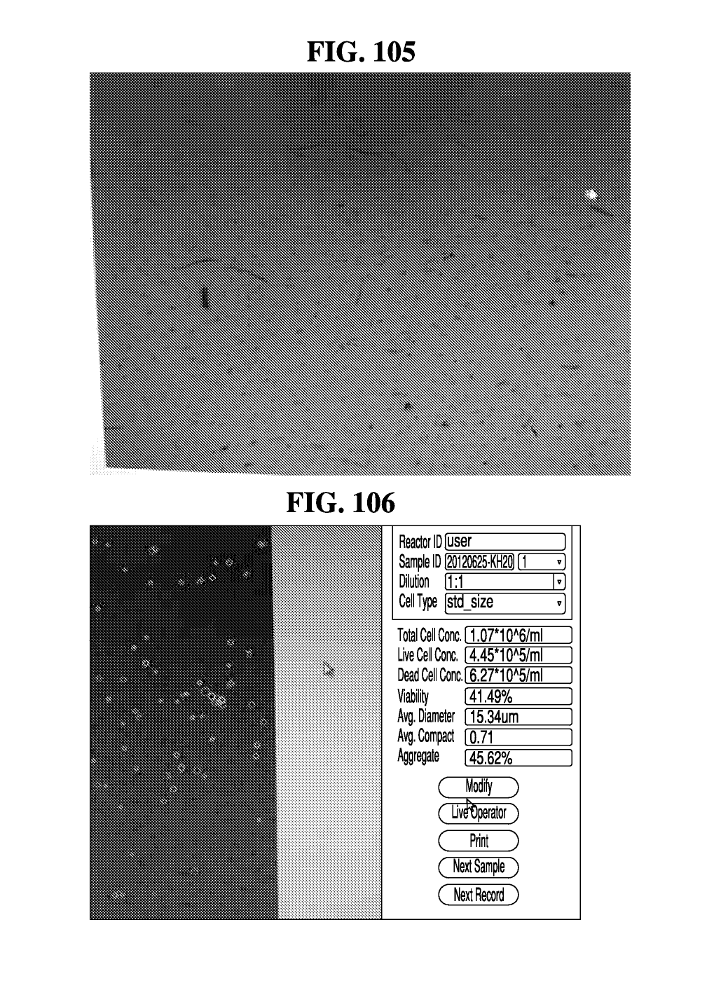

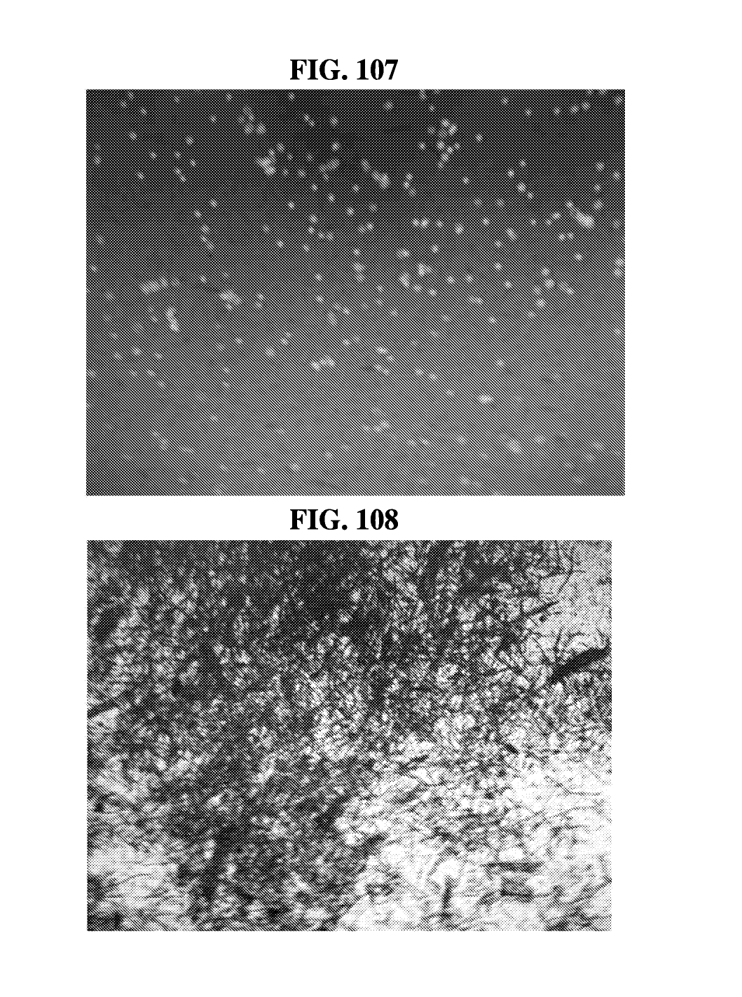

FIGS. 105, 106 and 10—KH203 medium sample number 4. Without Tryptophan.

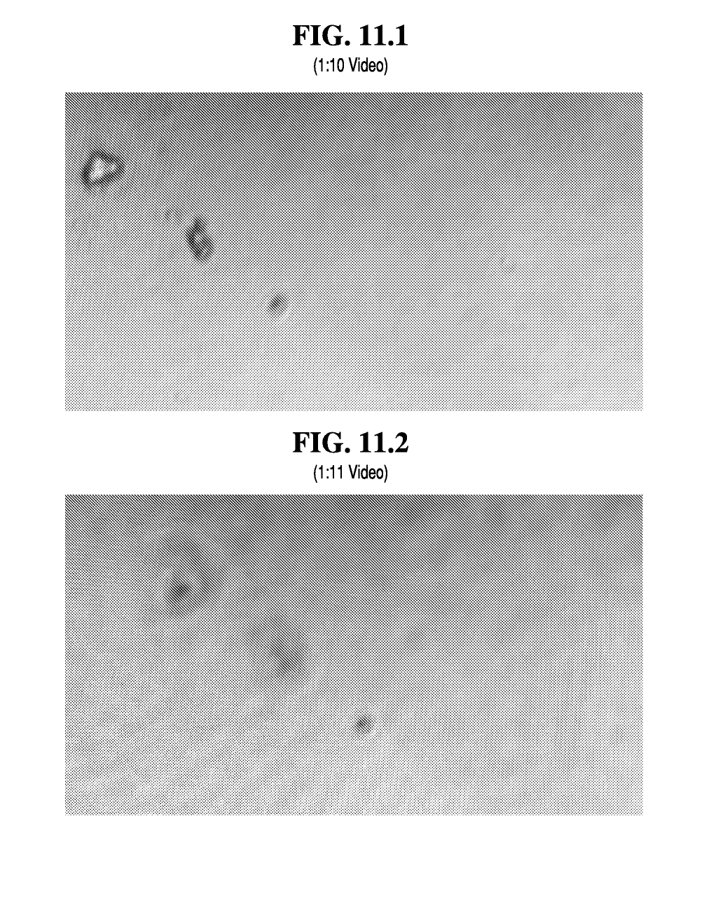

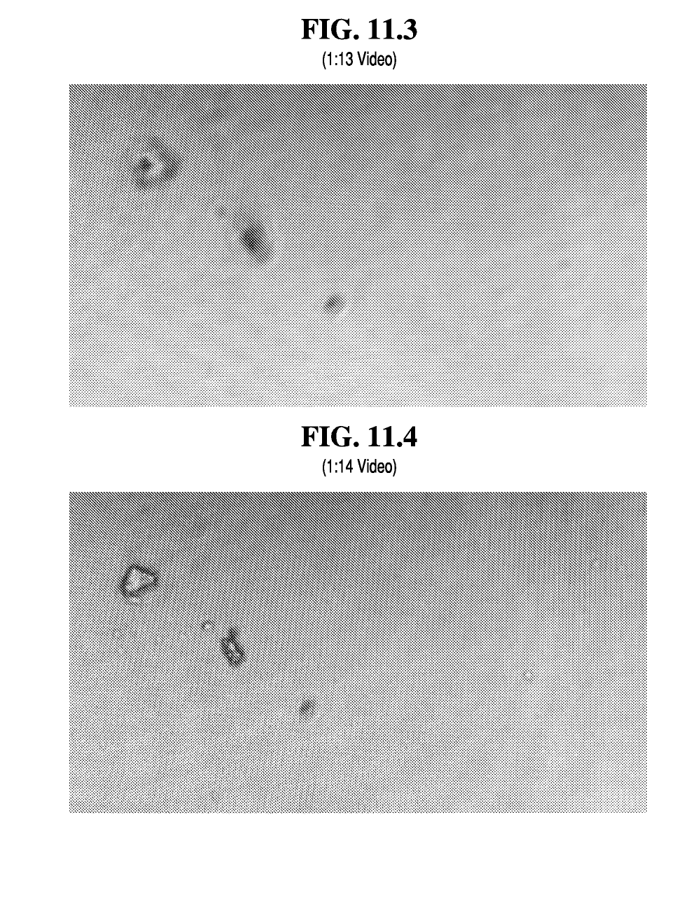

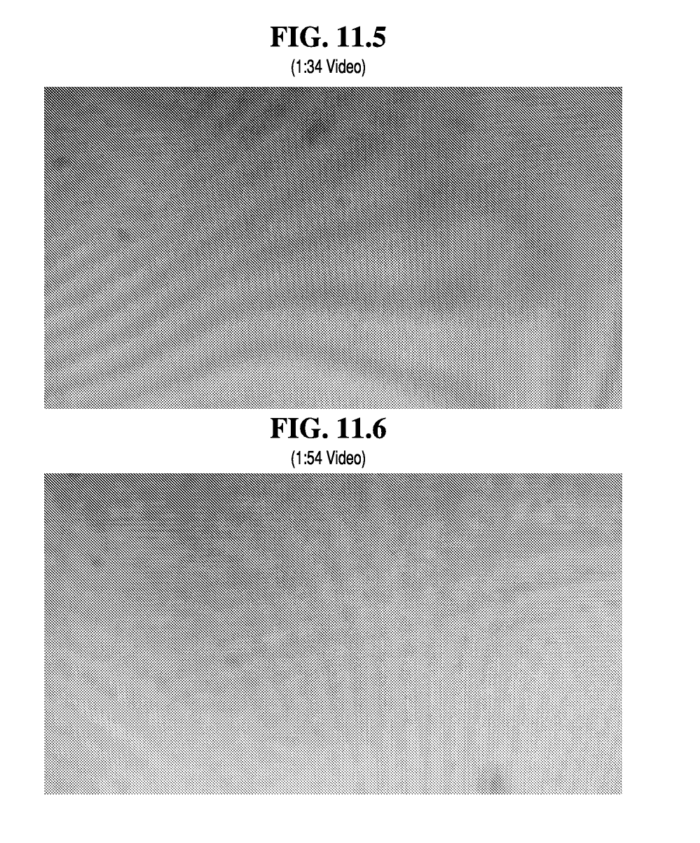

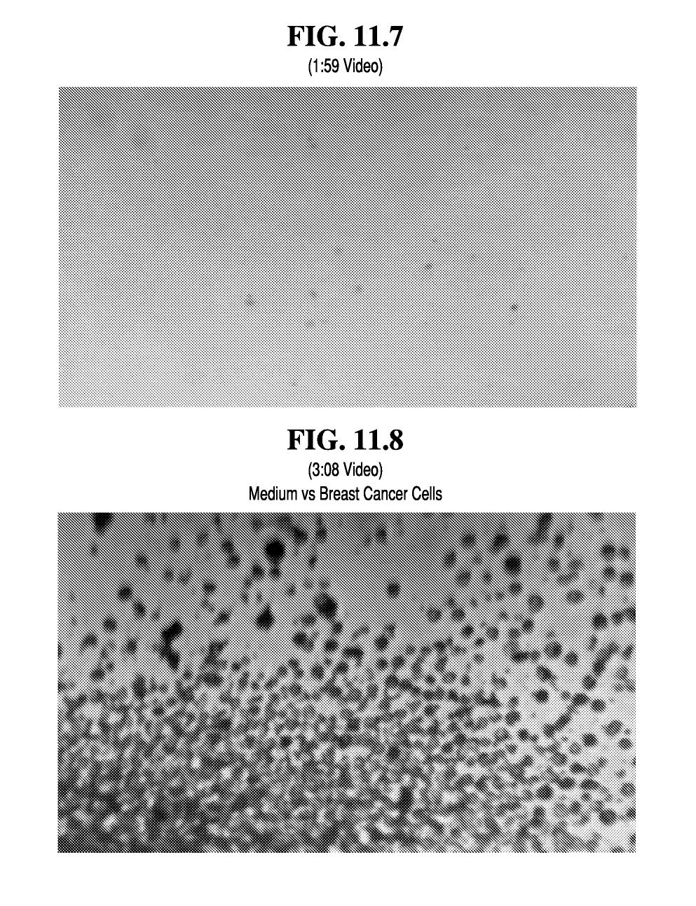

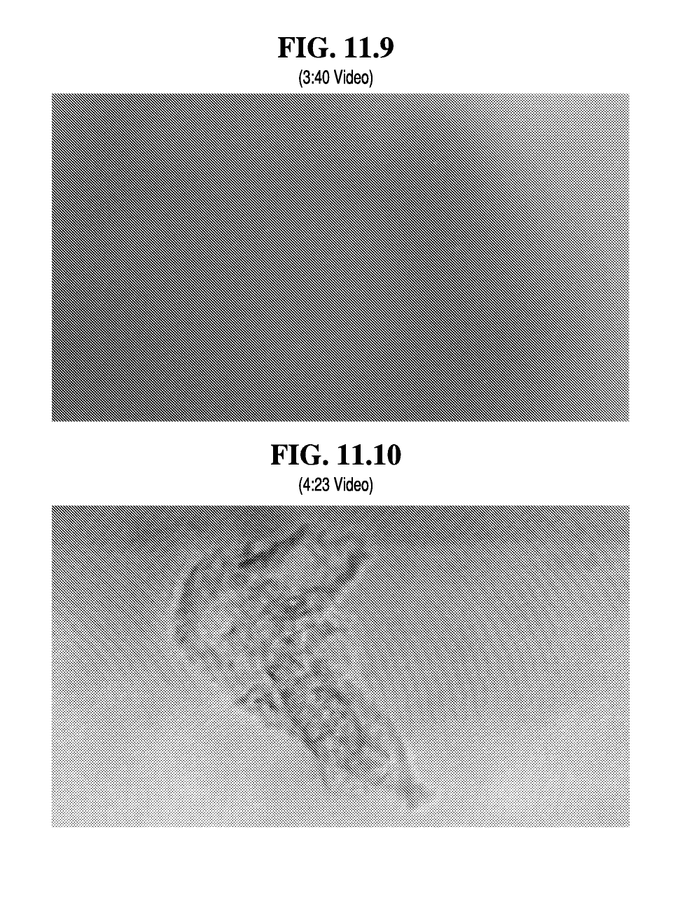

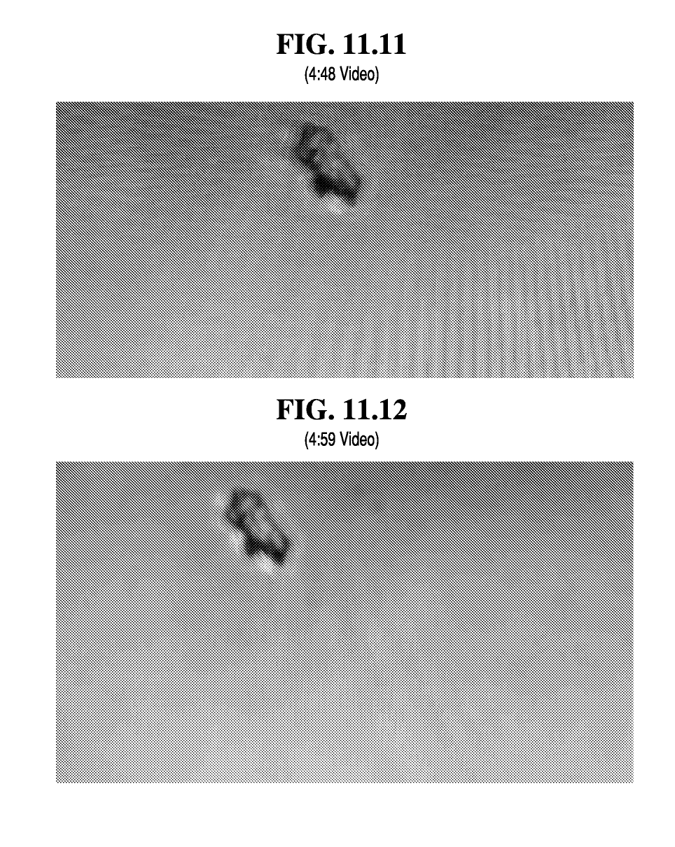

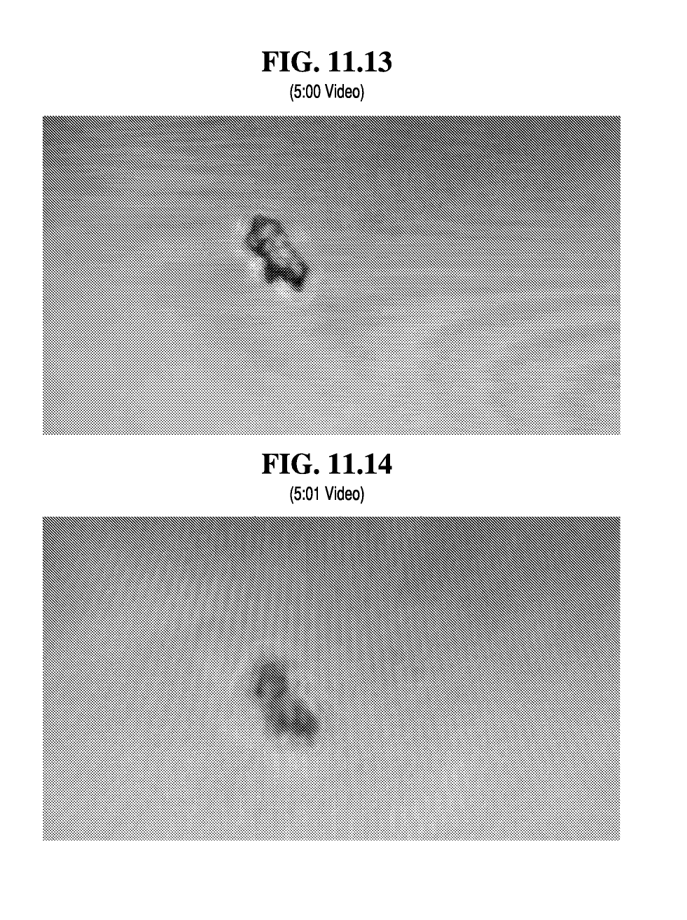

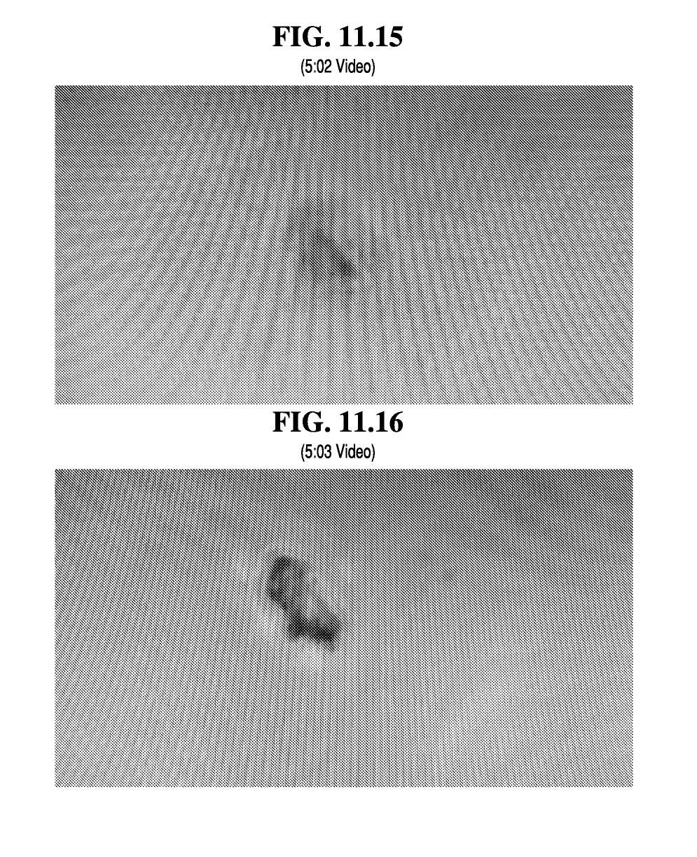

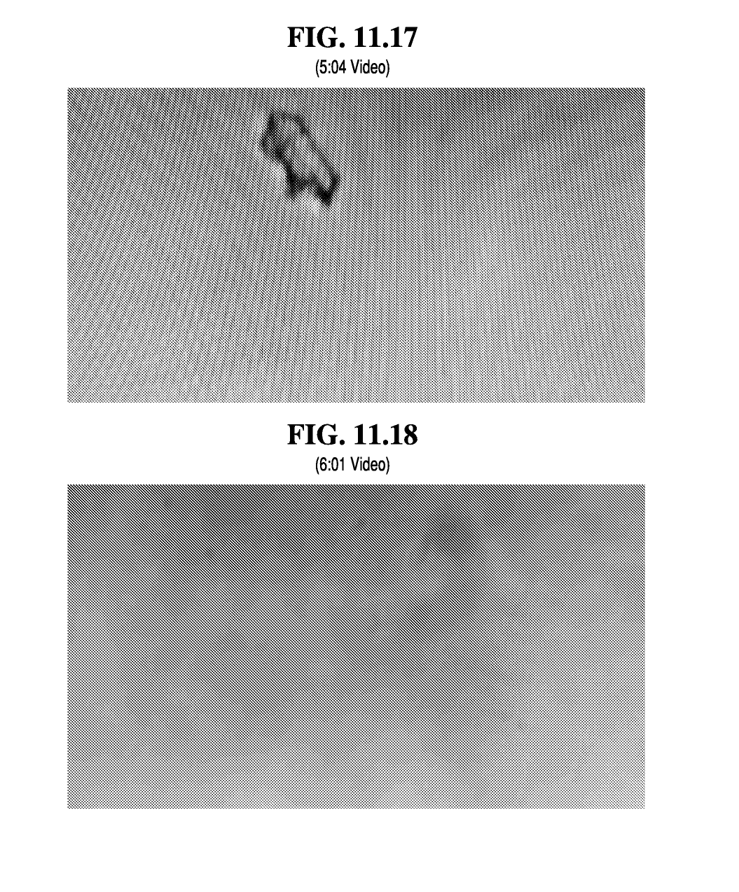

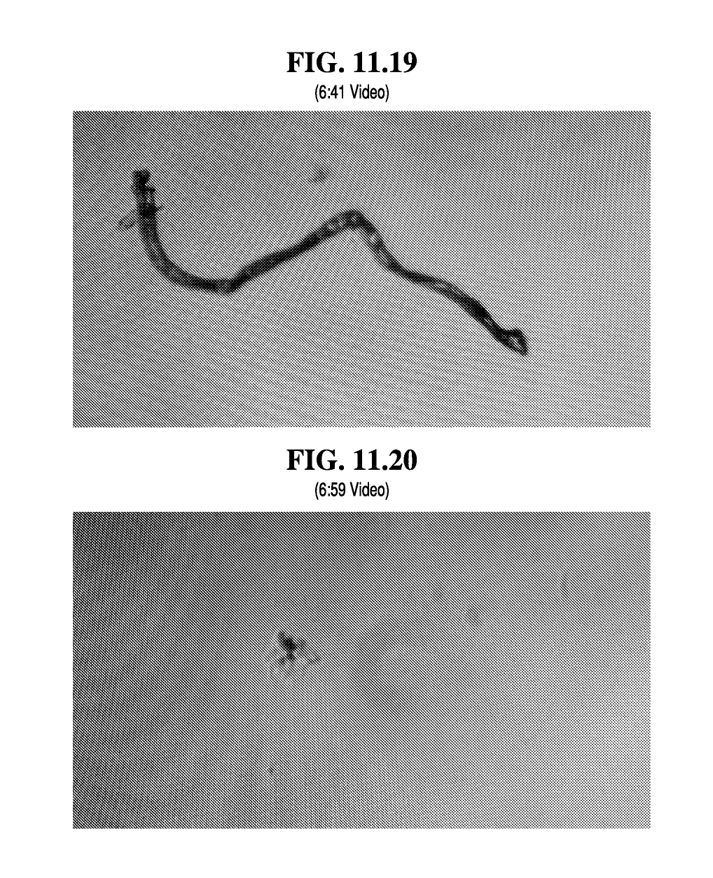

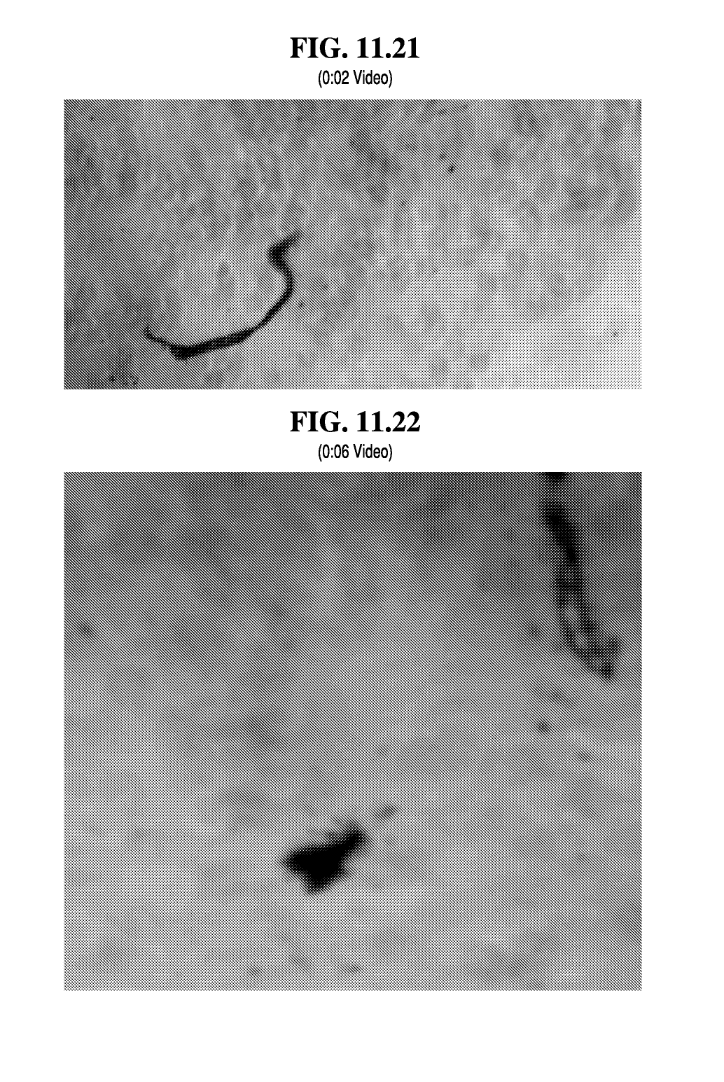

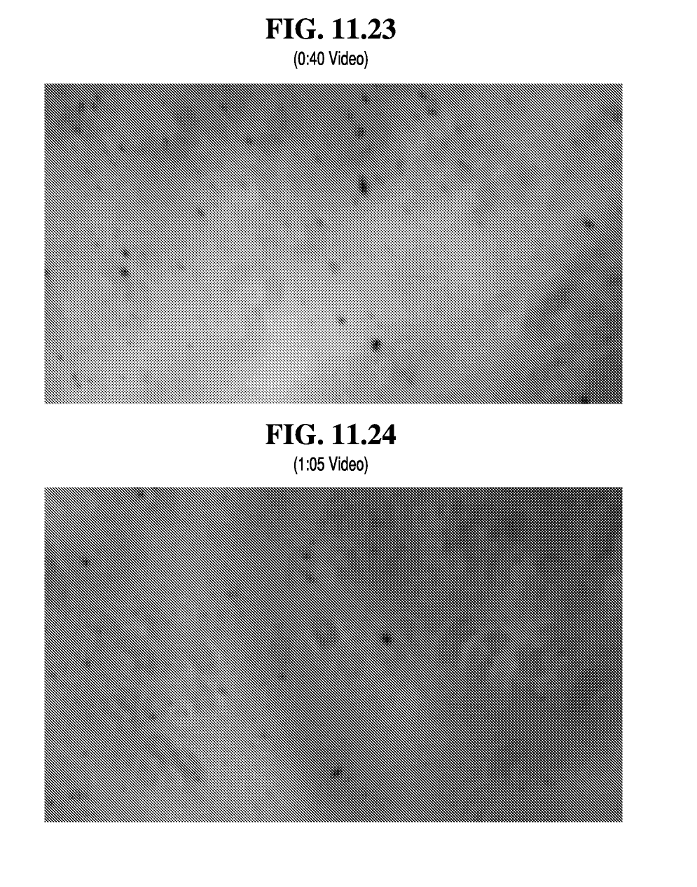

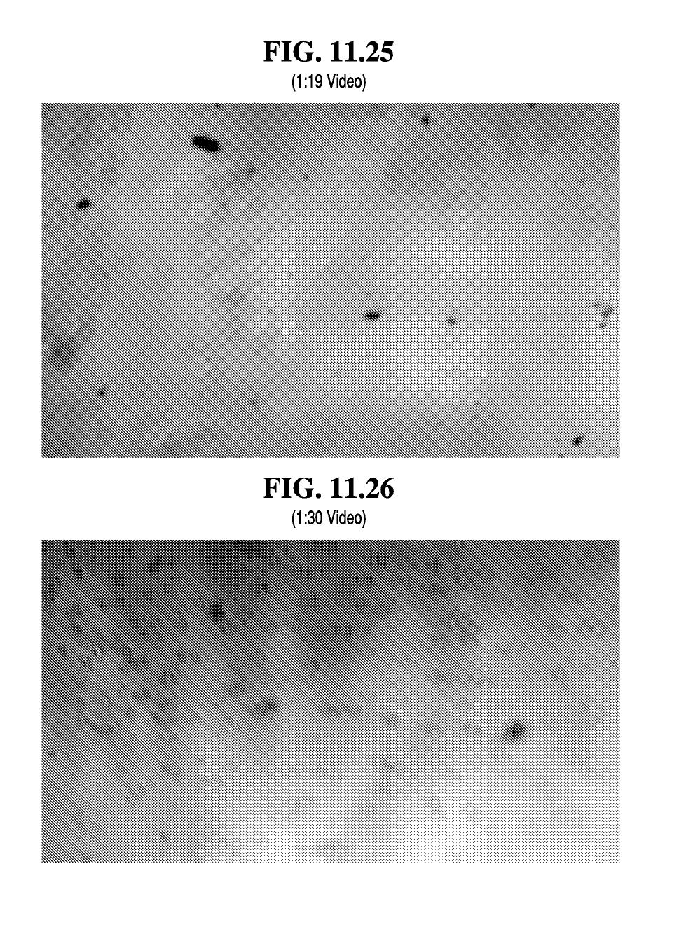

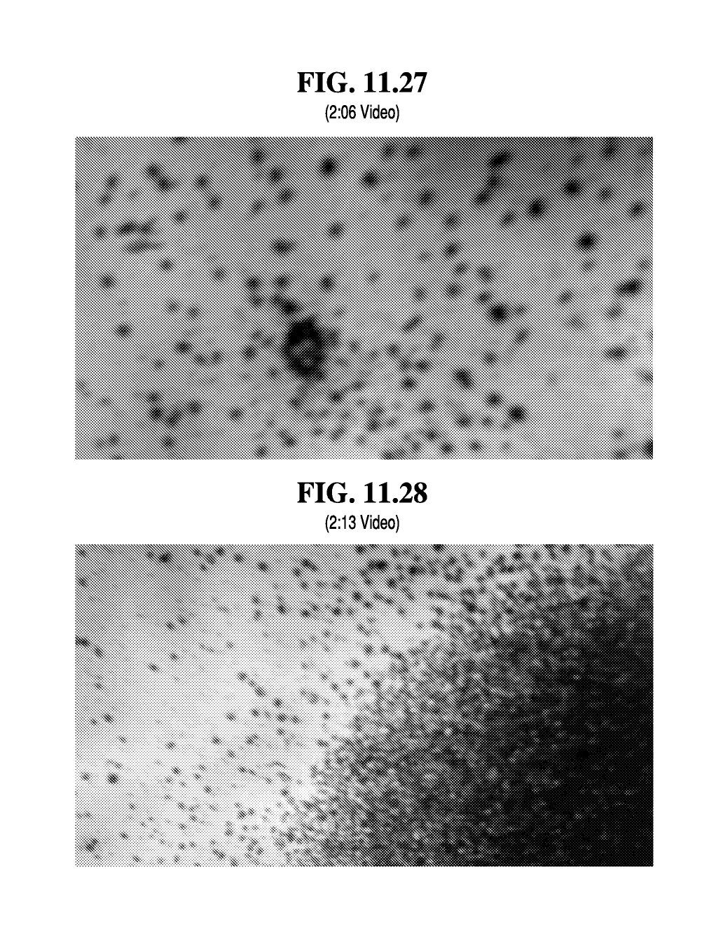

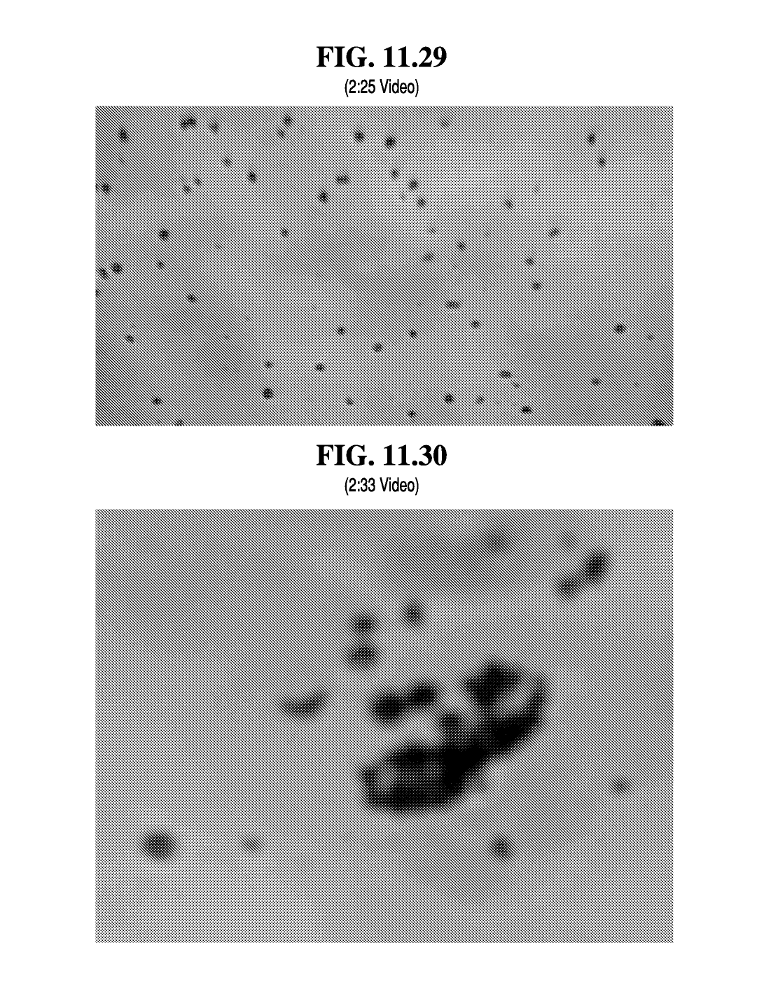

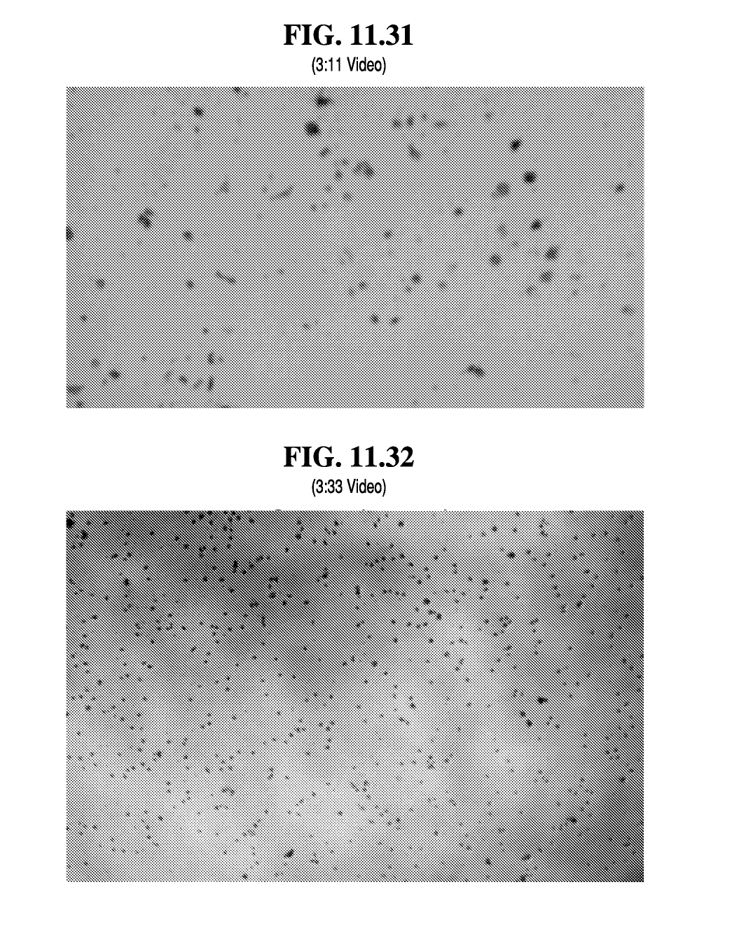

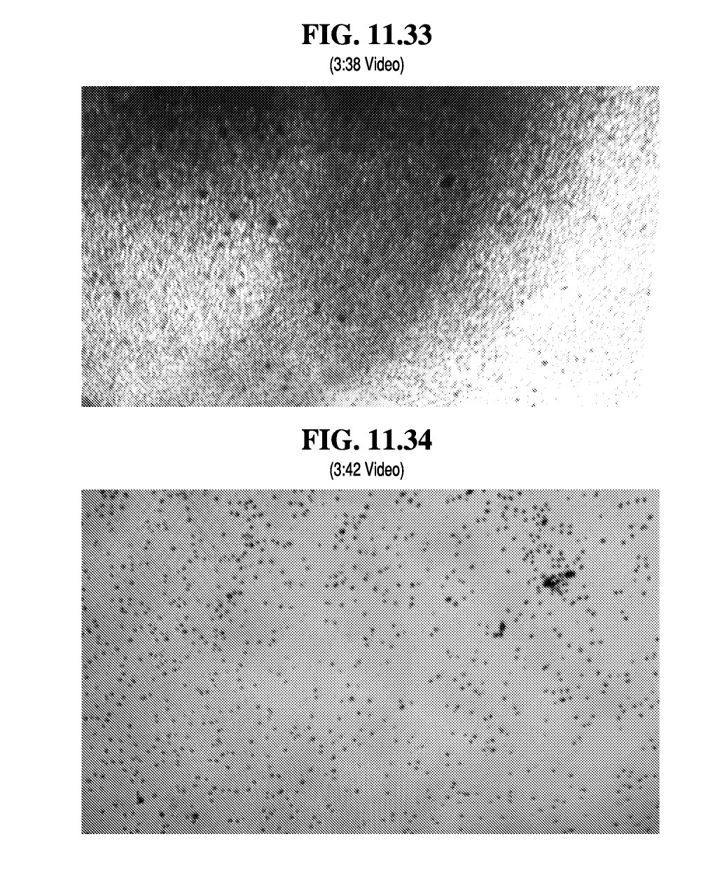

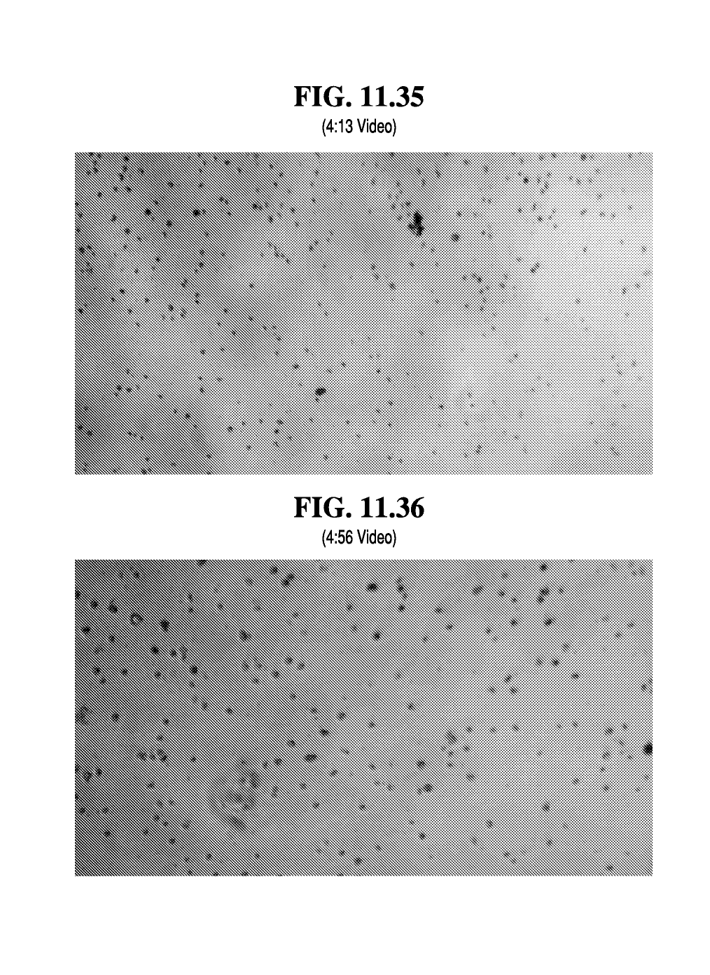

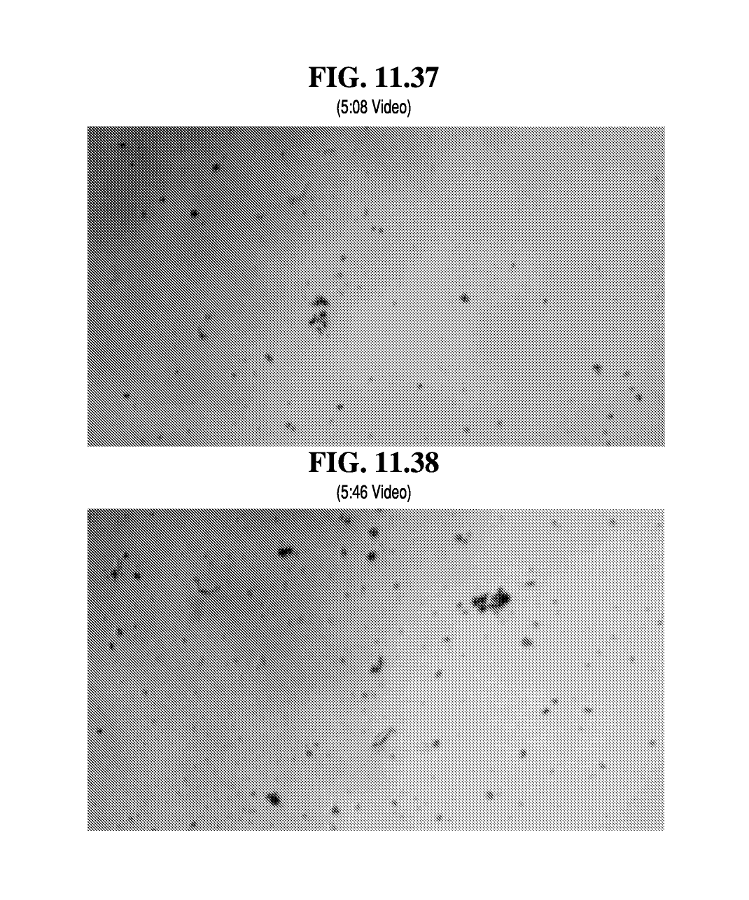

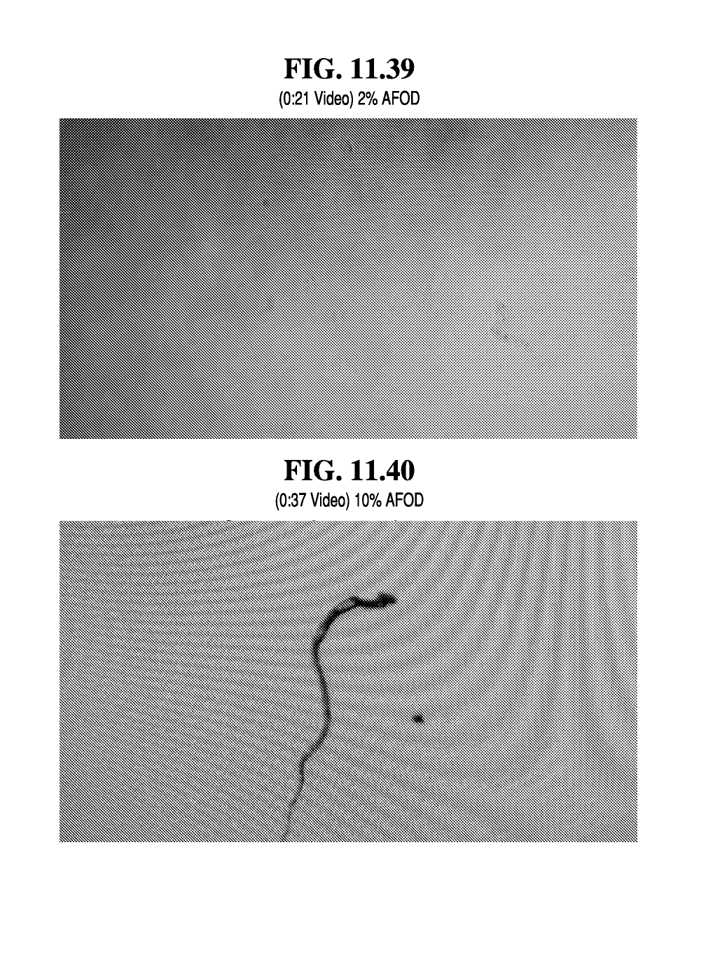

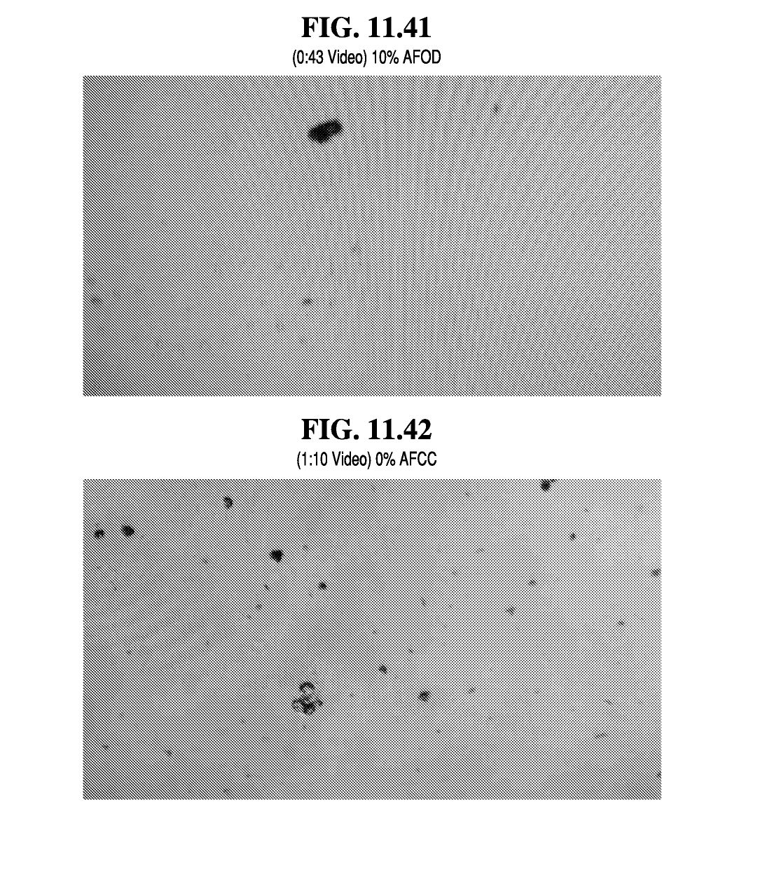

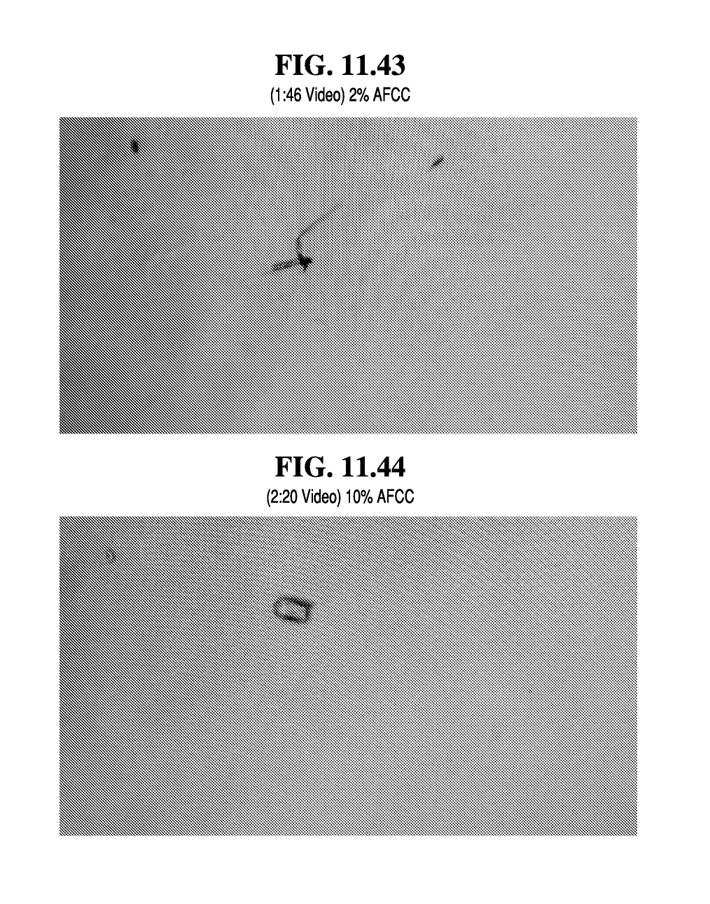

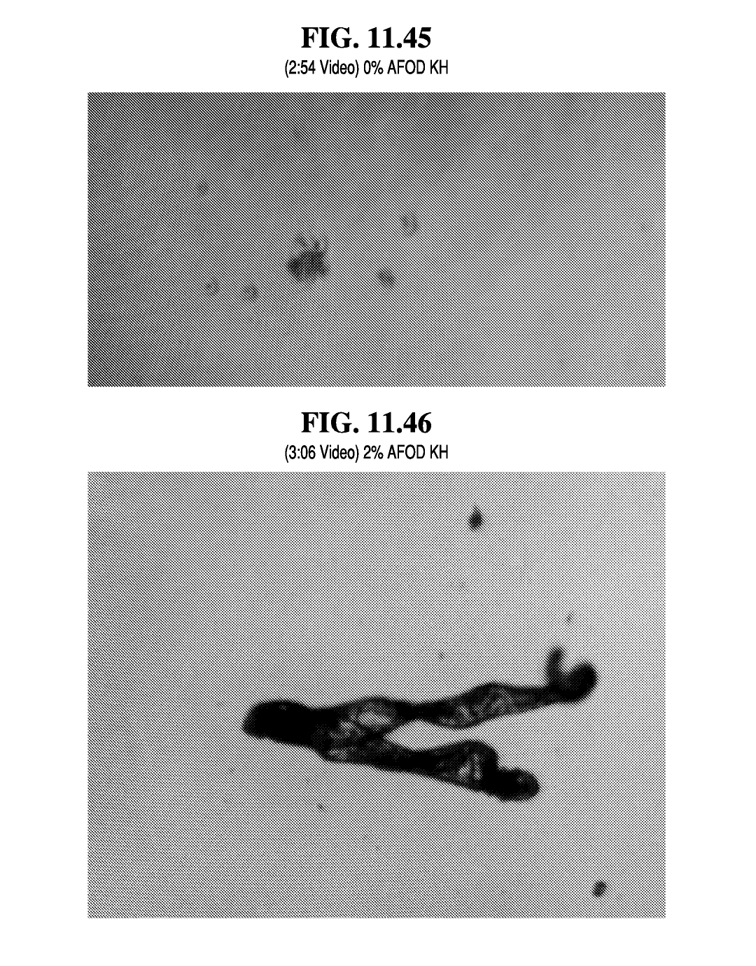

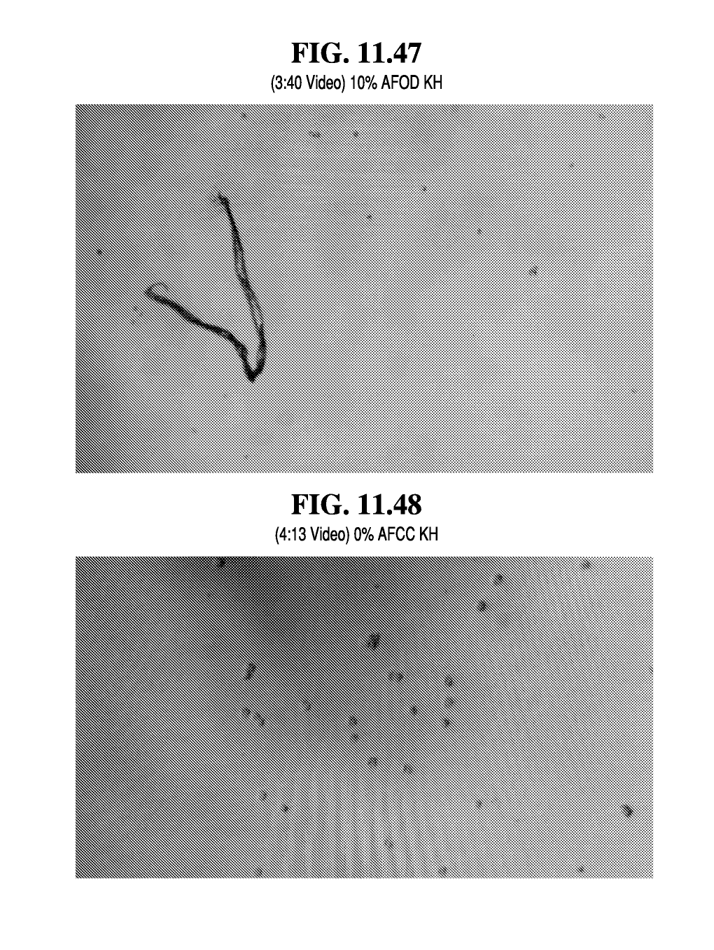

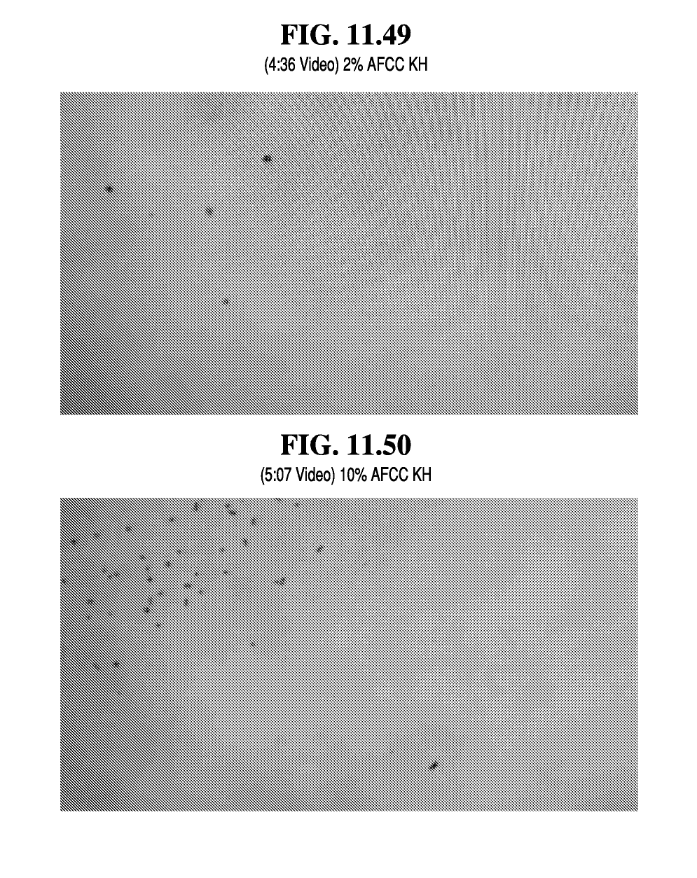

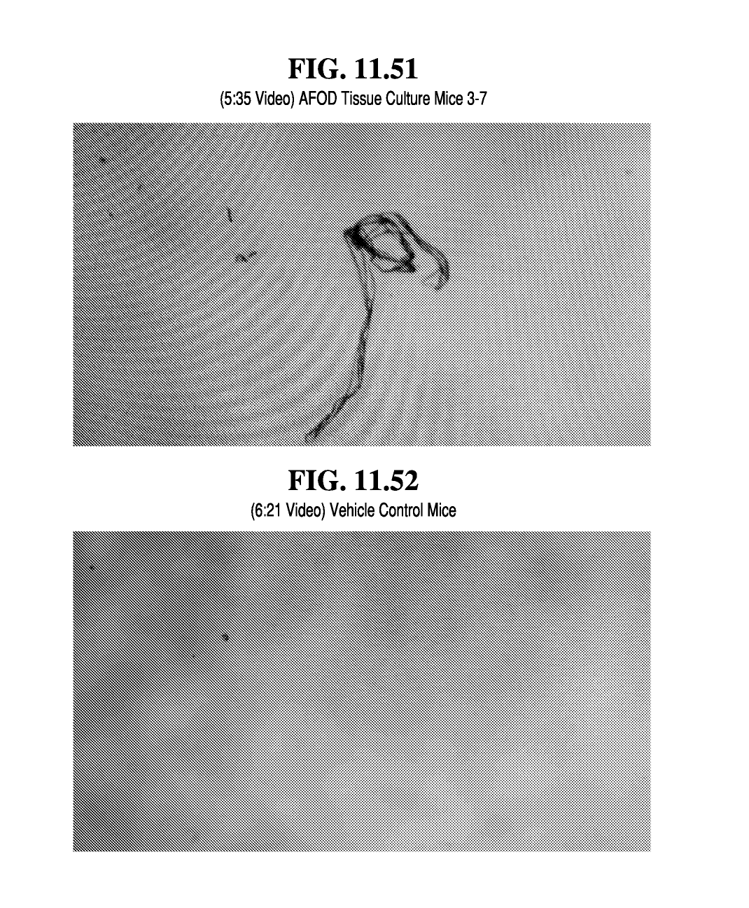

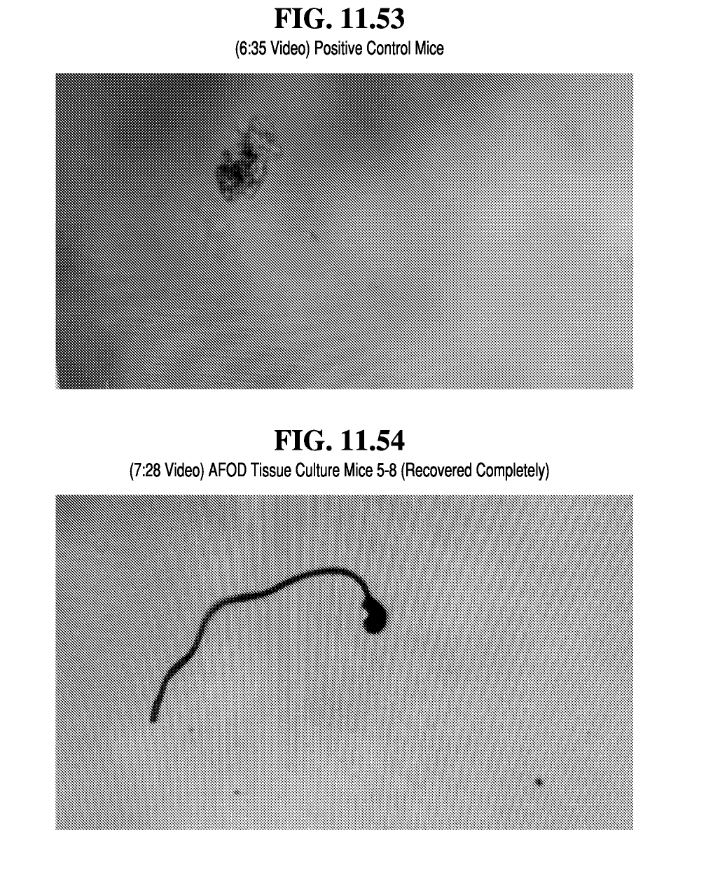

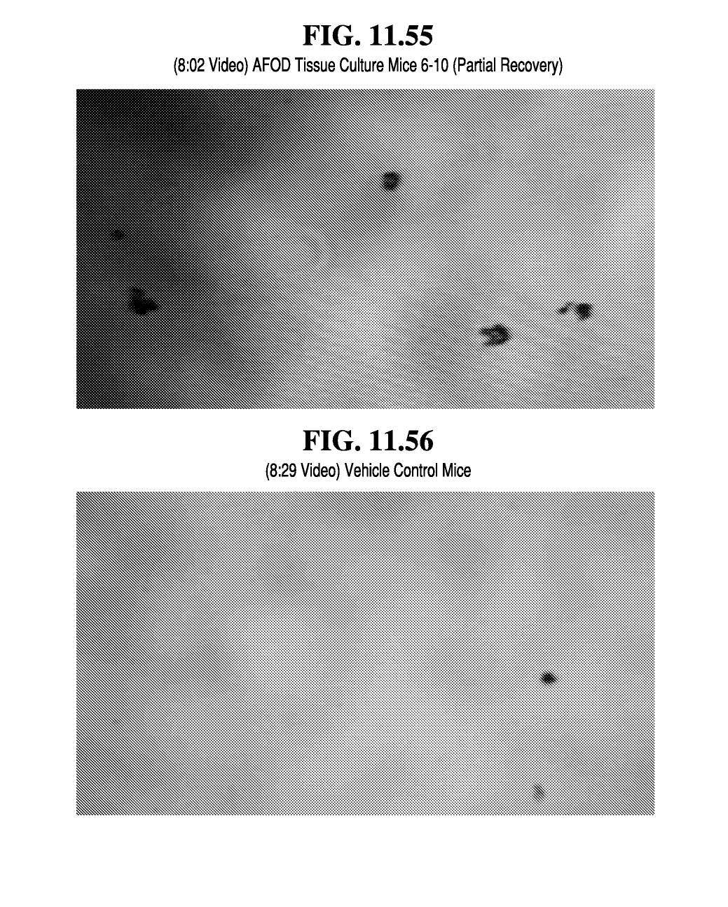

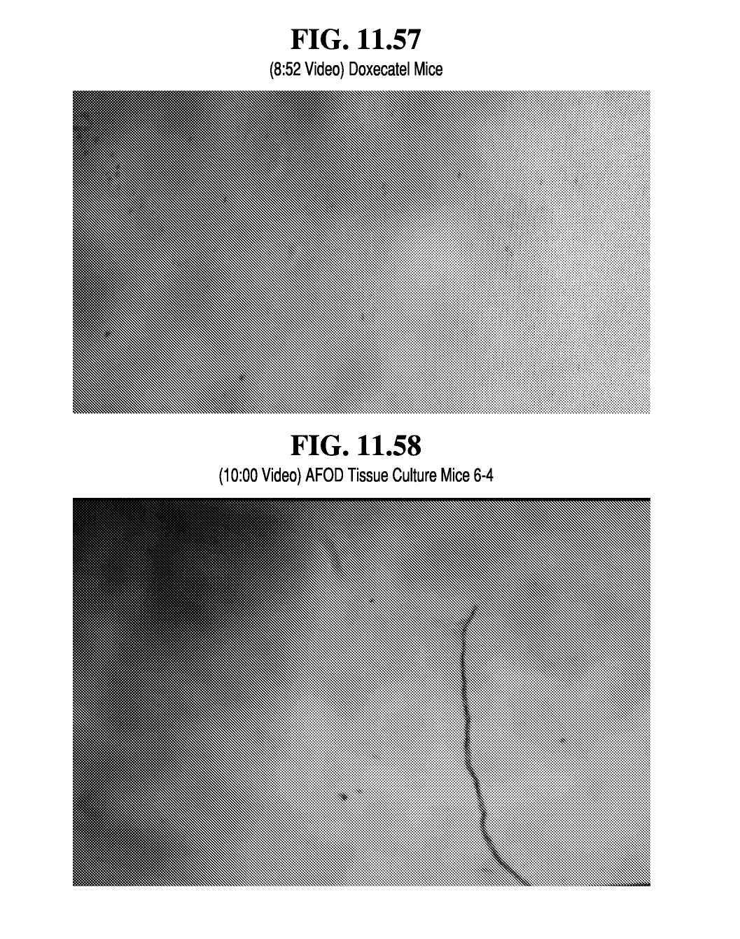

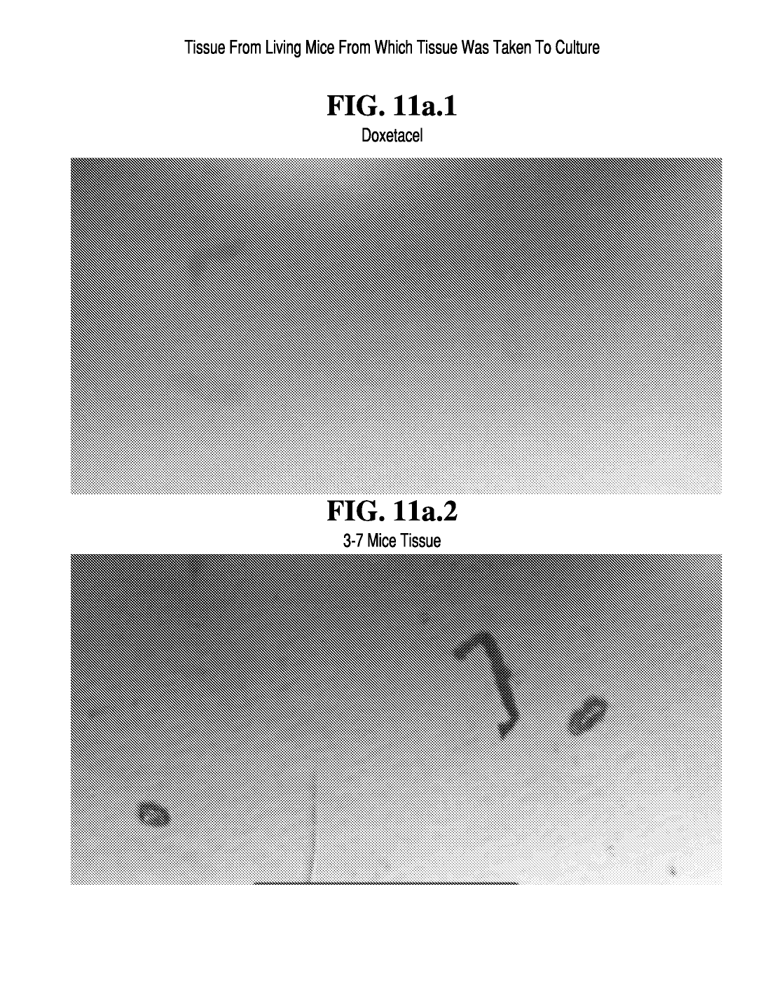

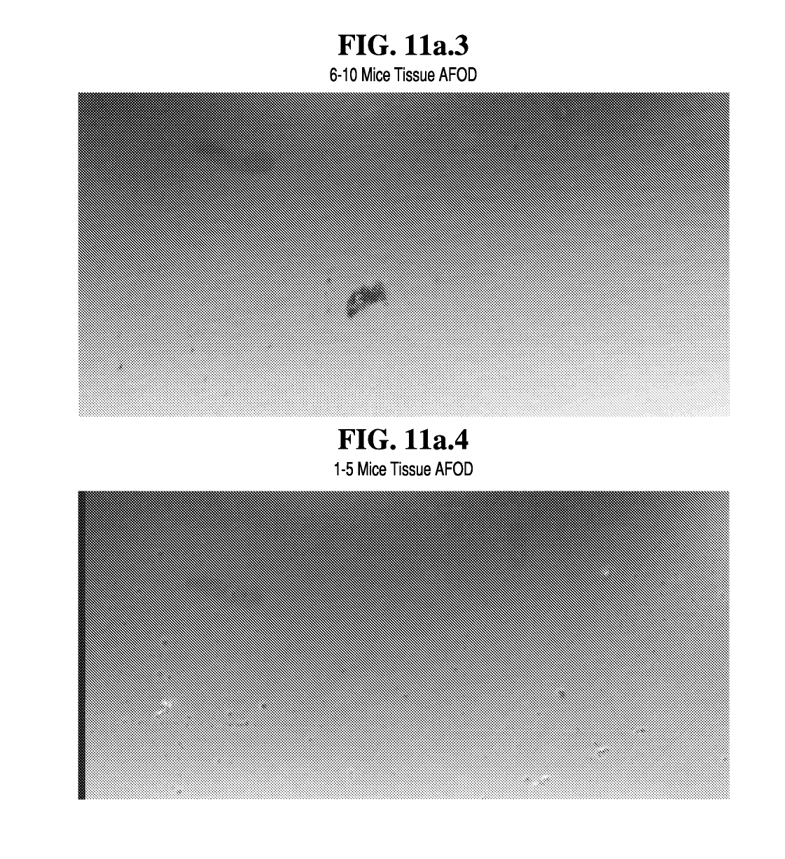

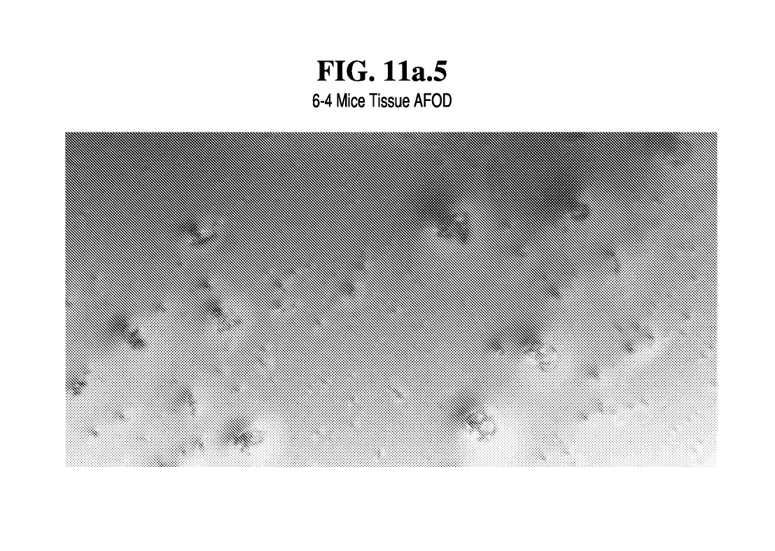

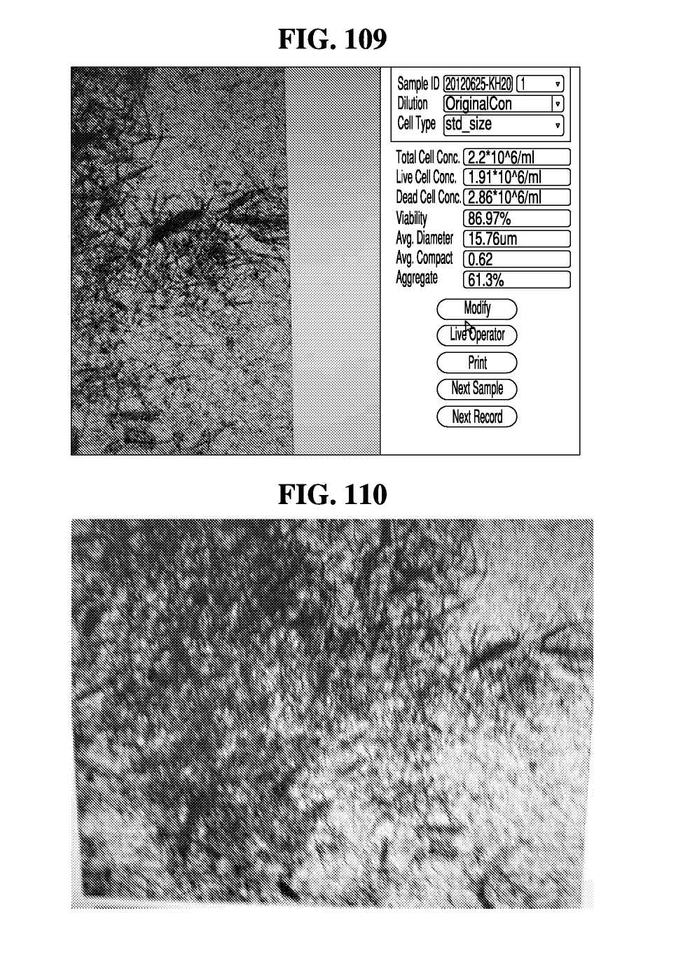

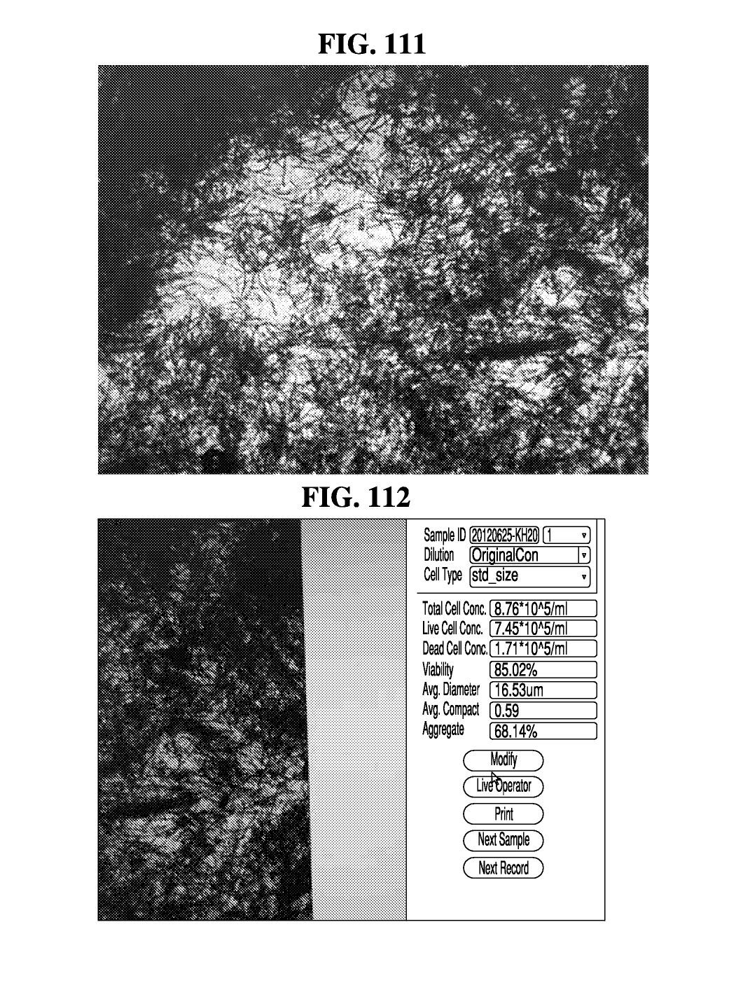

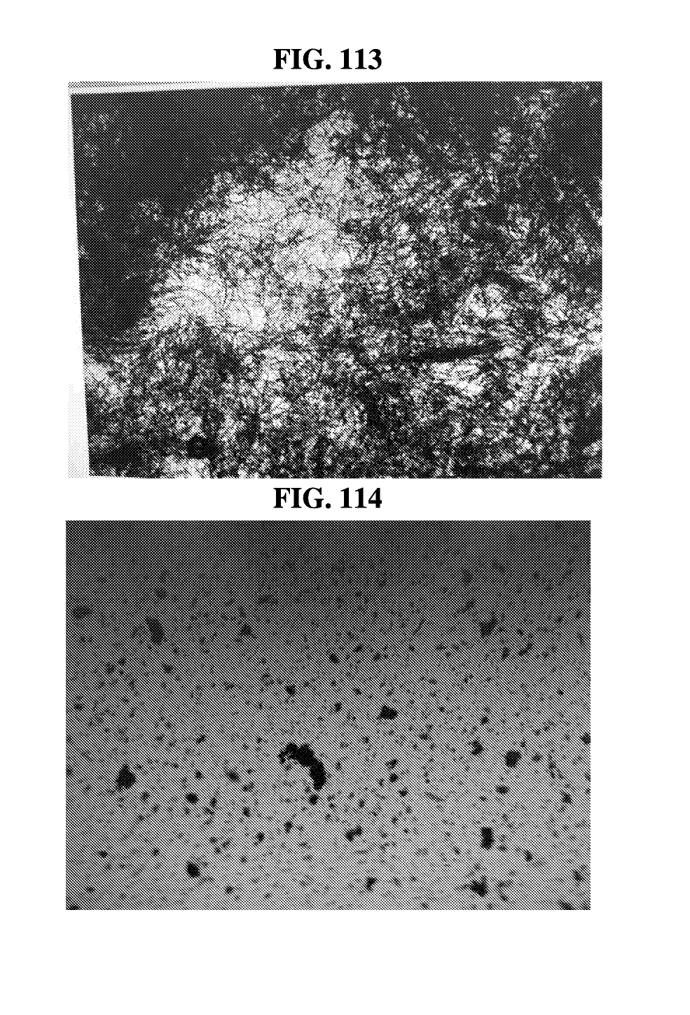

FIGS. 108, 109 and 11—KH203 medium sample number 5. Without Tryptophan.

FIGS. 111, 112 and 11—KH 204 medium contains 16 g of paste of Alaskan Crab with 300 mL of WFI. Sample number 1, Tryptophan is added as a stabilizer.

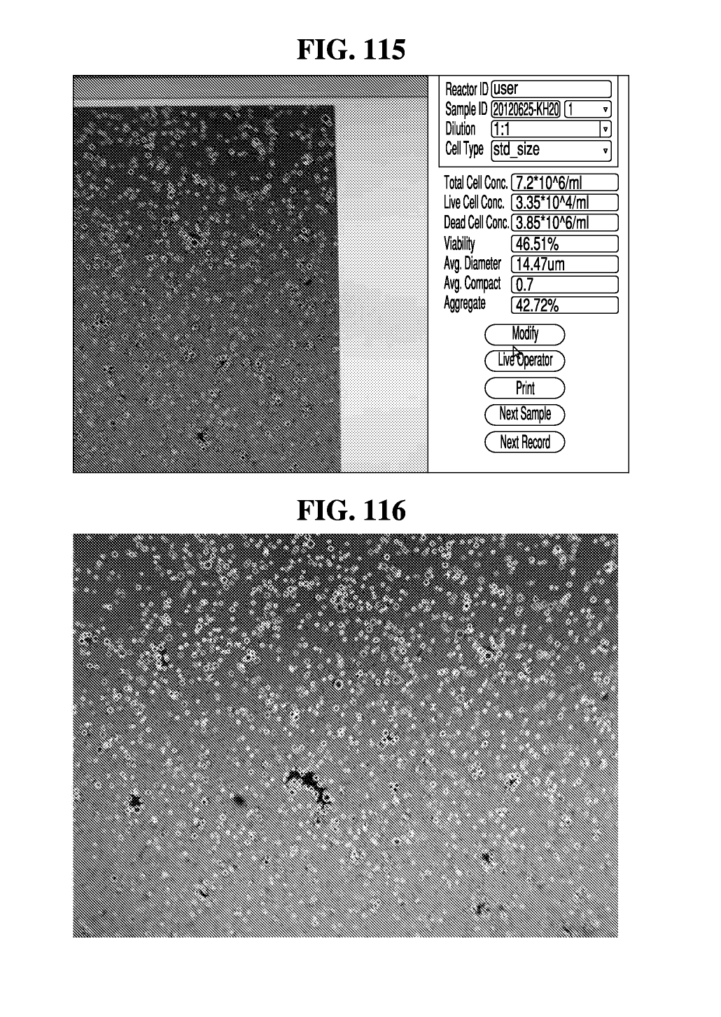

FIGS. 114, 115 and 11—KH204 medium sample number 2. Tryptophan is added as a stabilizer.

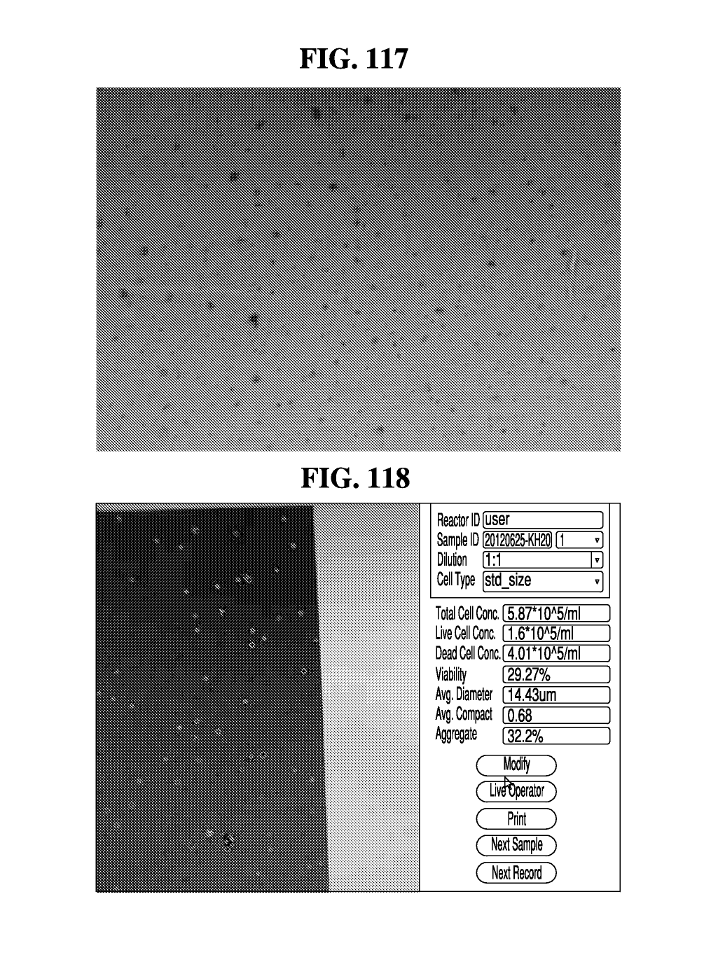

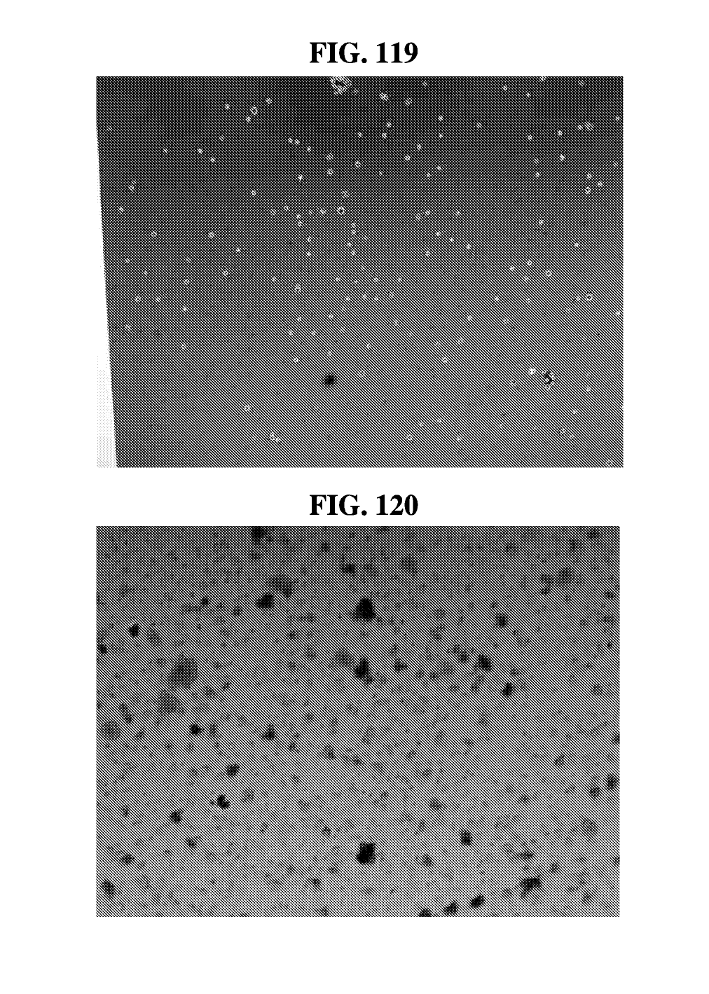

FIGS. 117, 118 and 11—KH204 medium sample number 3. Tryptophan is added as a stabilizer.

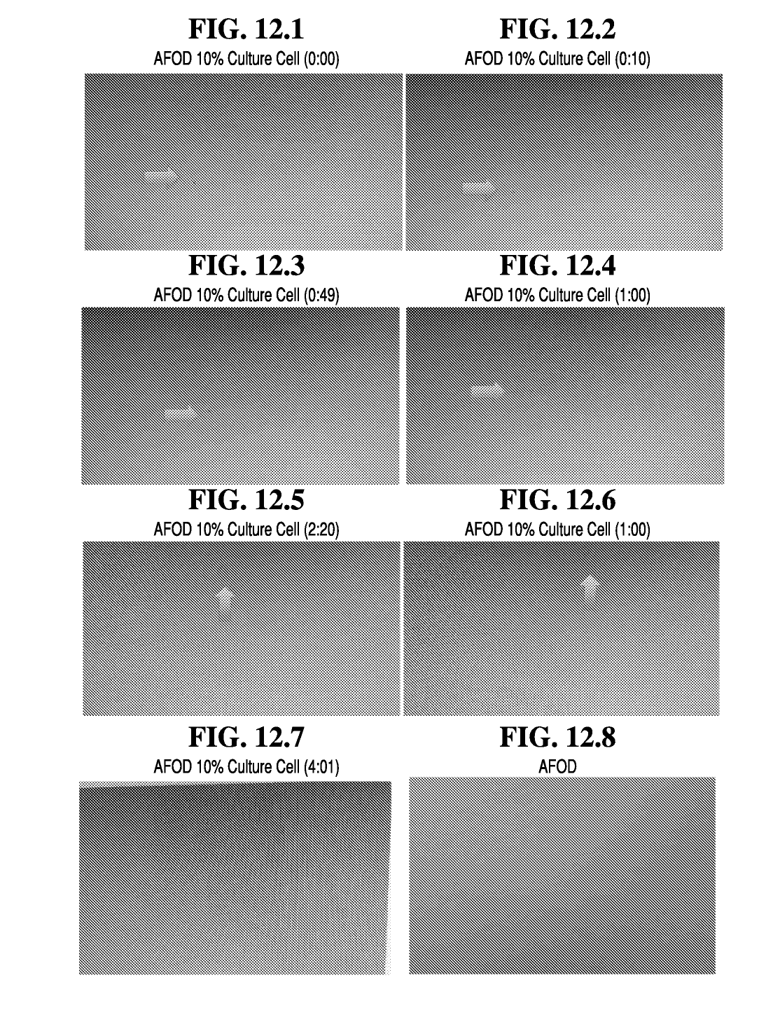

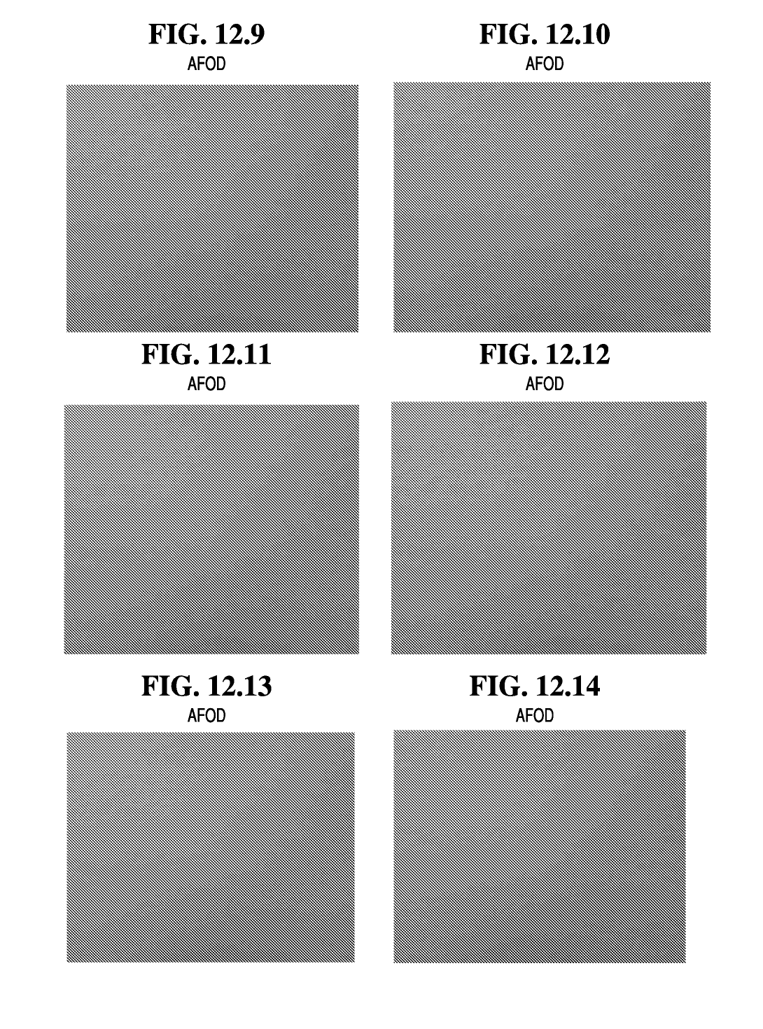

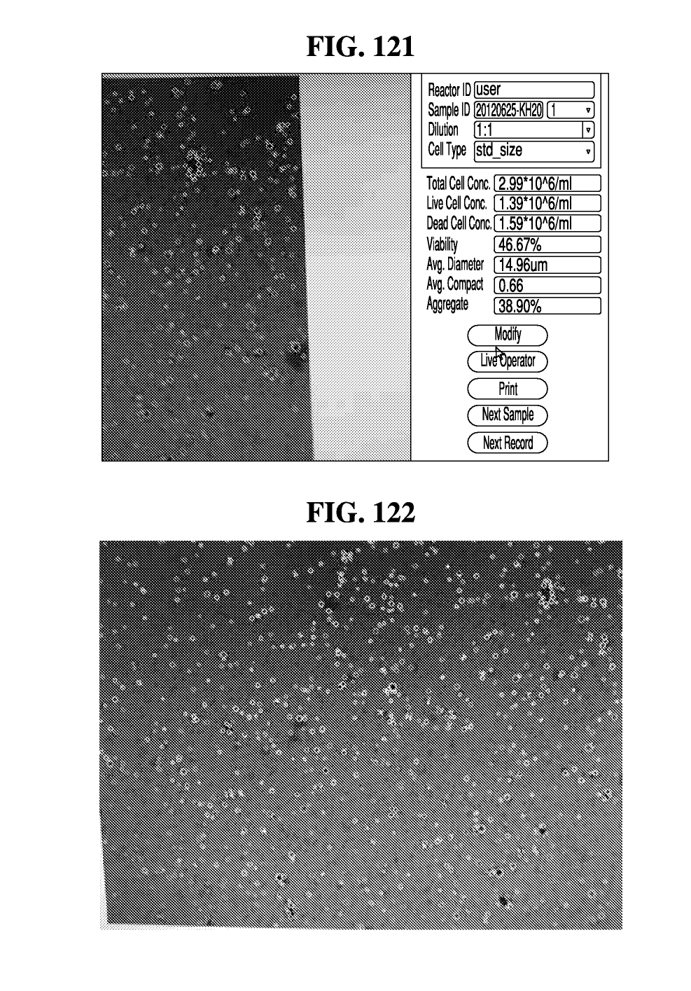

FIGS. 120, 121 and 12—KH204 medium sample number 4. Without Tryptophan.

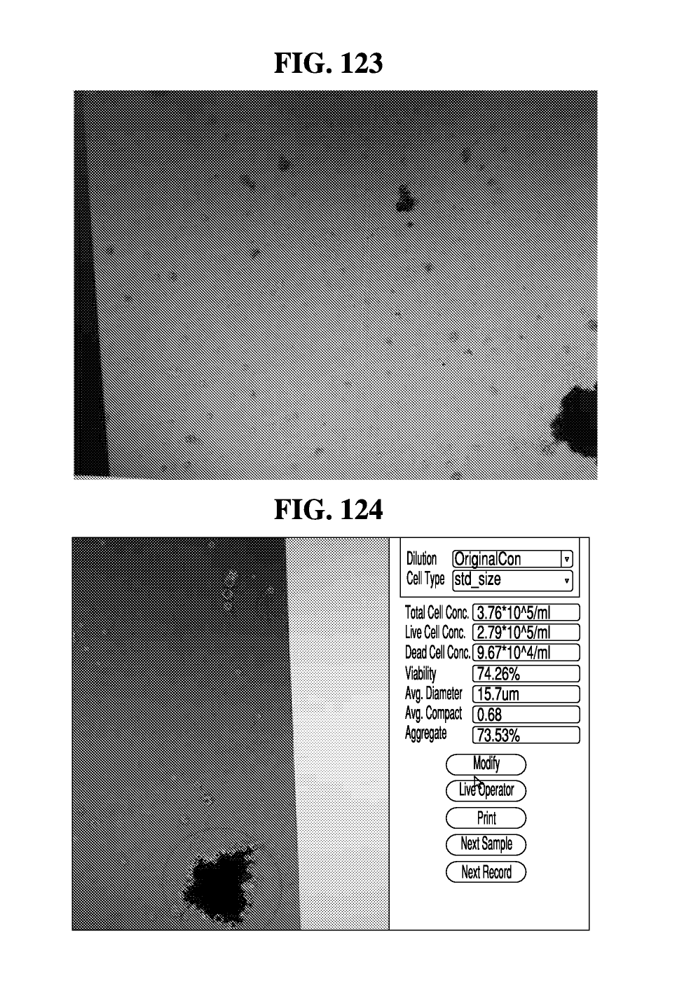

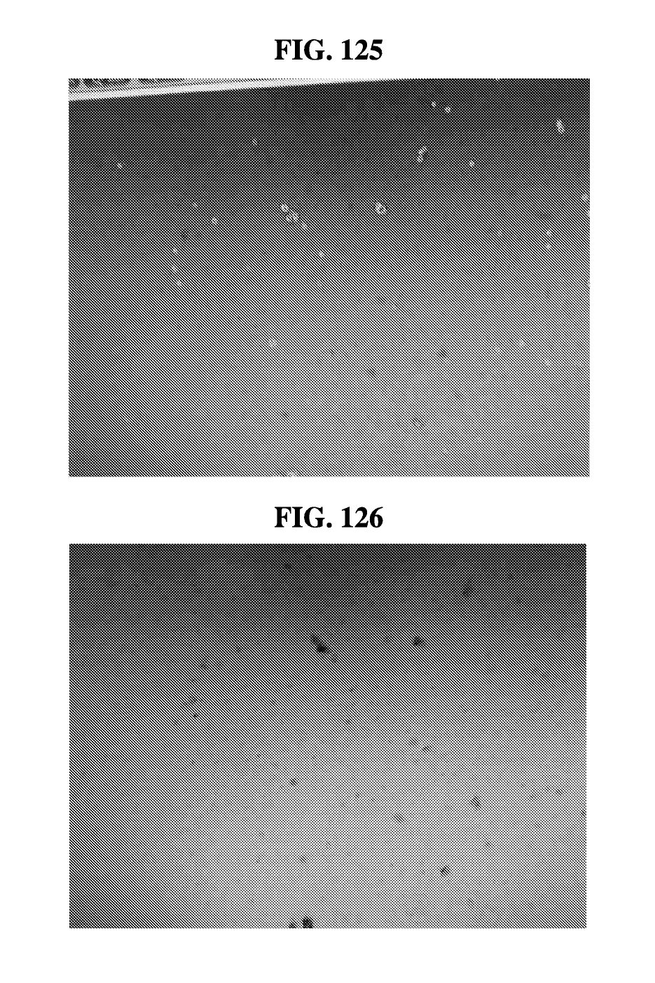

FIGS. 123, 124 and 12—KH204 medium sample number 5. Without Tryptophan.

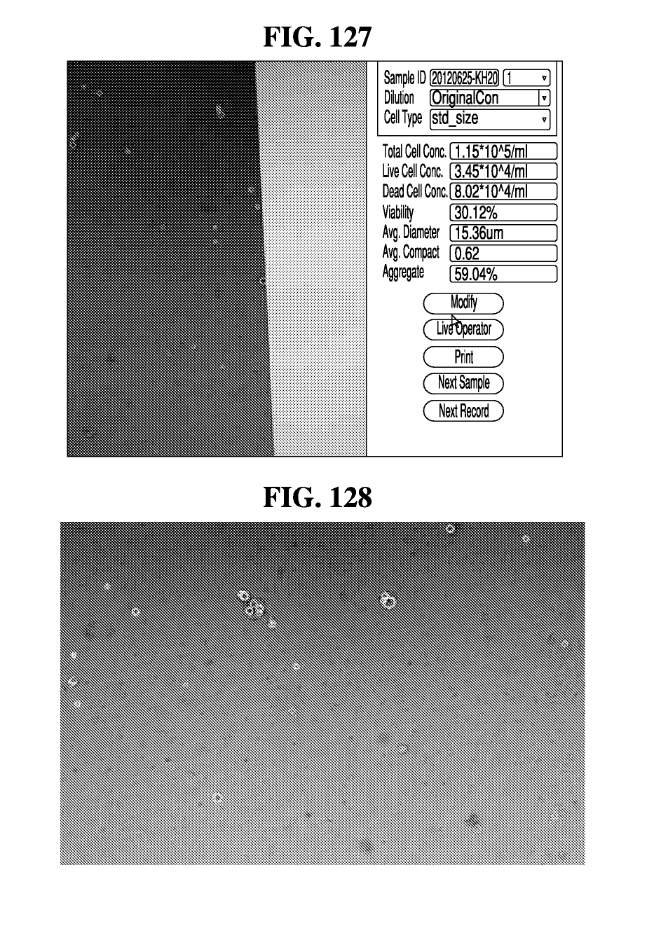

FIGS. 126, 127 and 12—KH 205 medium contains 24.4 g of paste of Pork with 500 mL of WFI. Sample number 1, Tryptophan is added as a stabilizer.

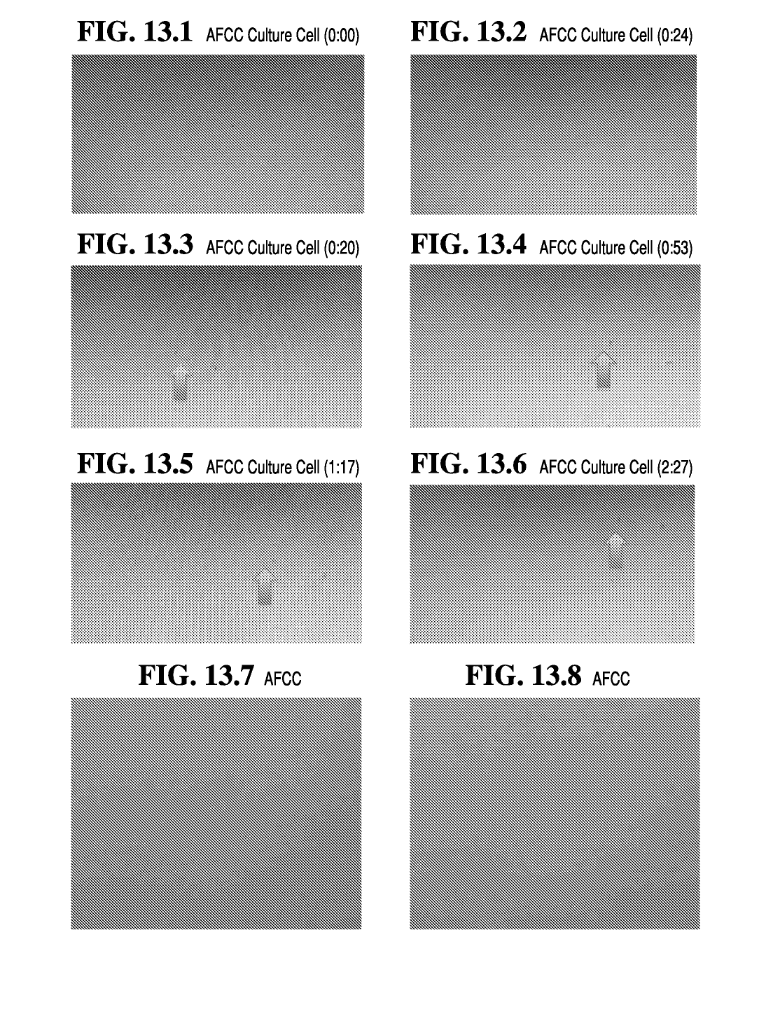

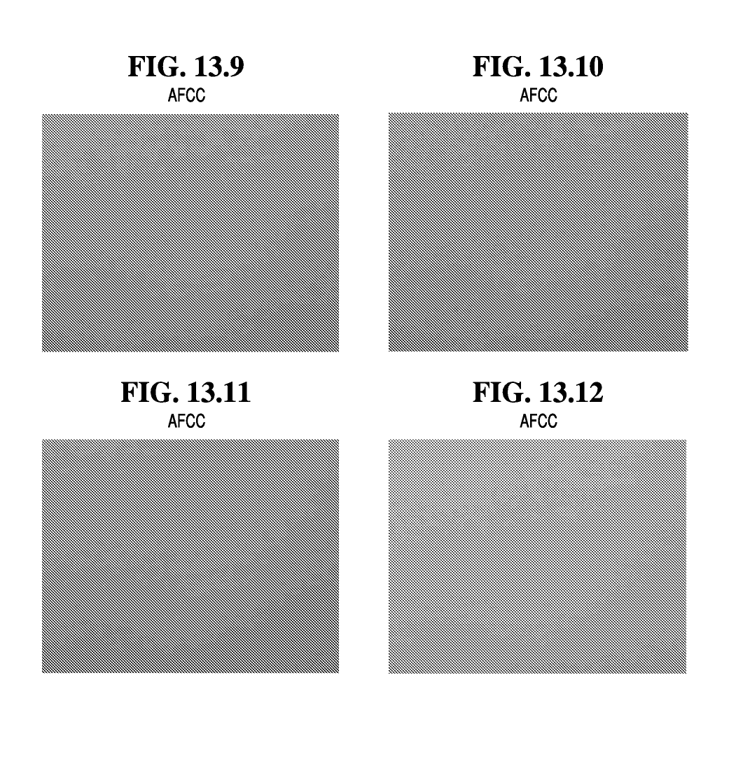

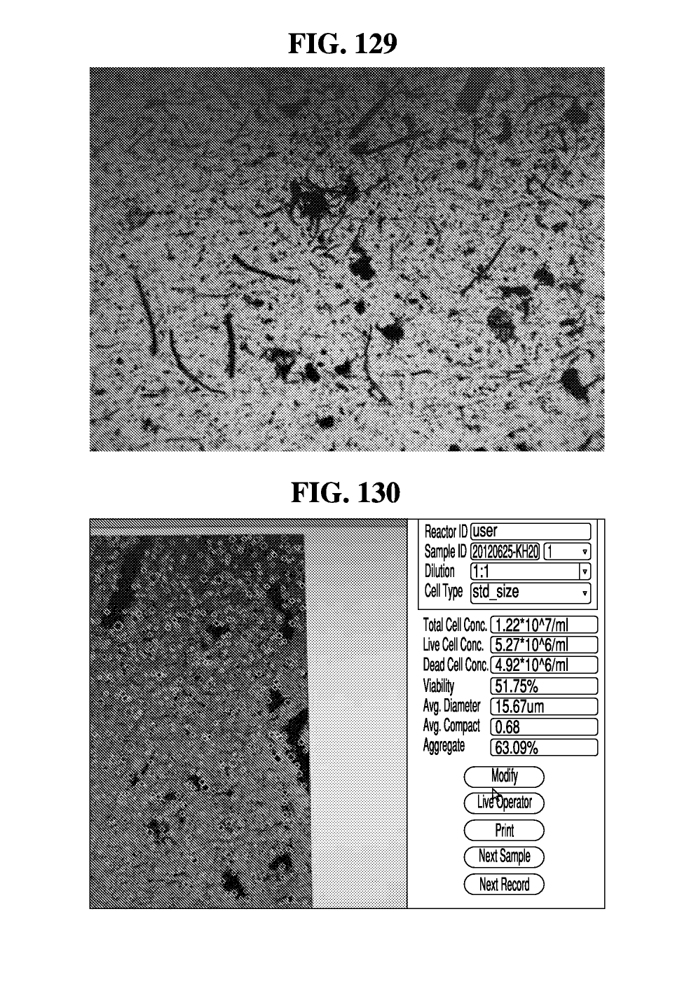

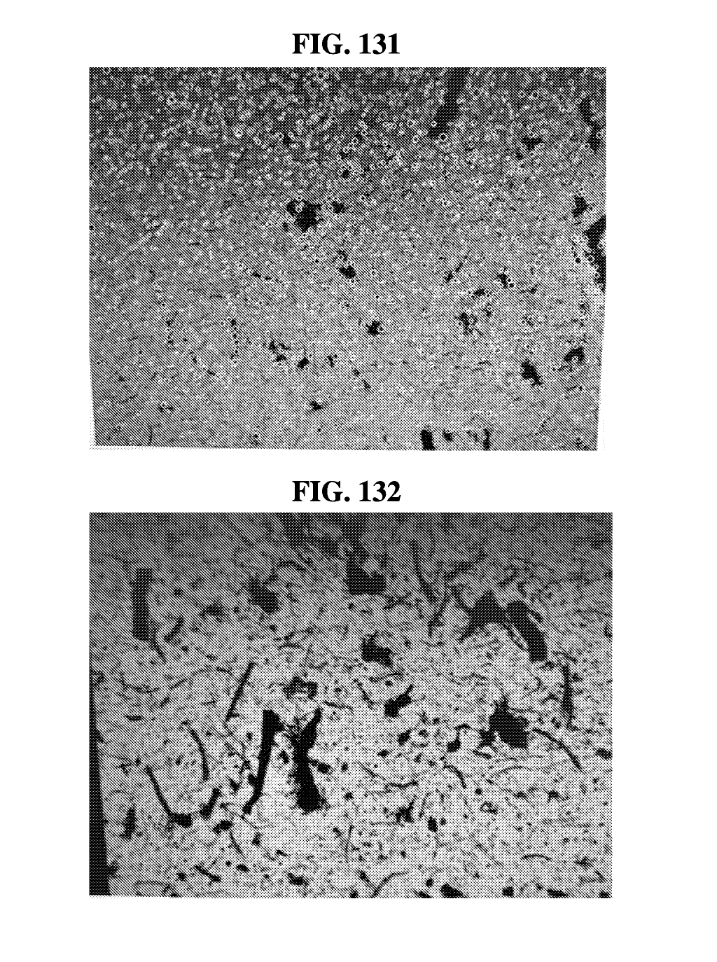

FIGS. 129, 130 and 13—KH205 medium sample number 2. Tryptophan is added as a stabilizer.

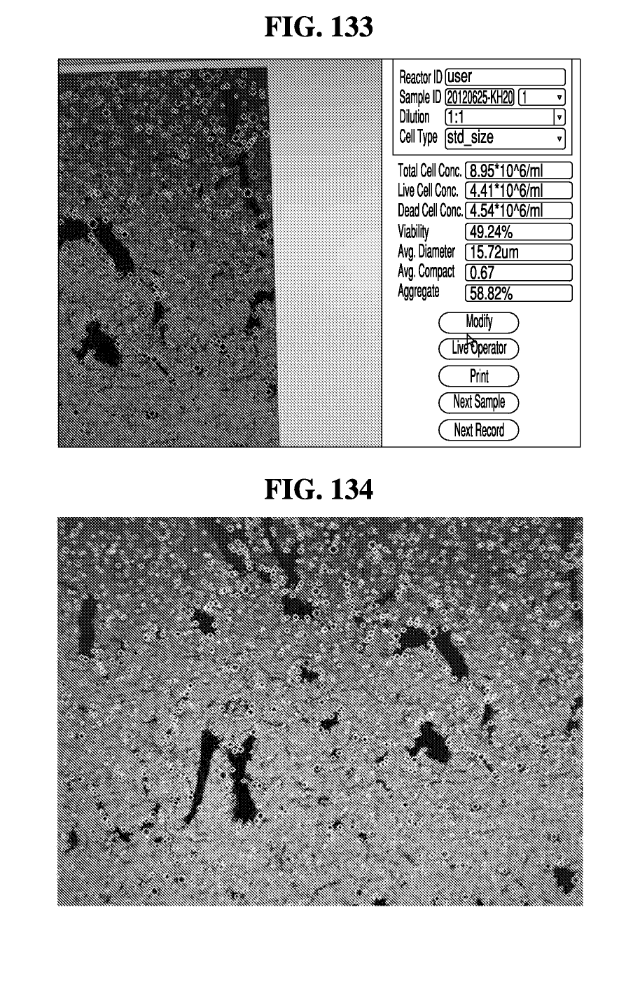

FIGS. 132, 133 and 13—KH205 medium sample number 3. Tryptophan is added as a stabilizer.

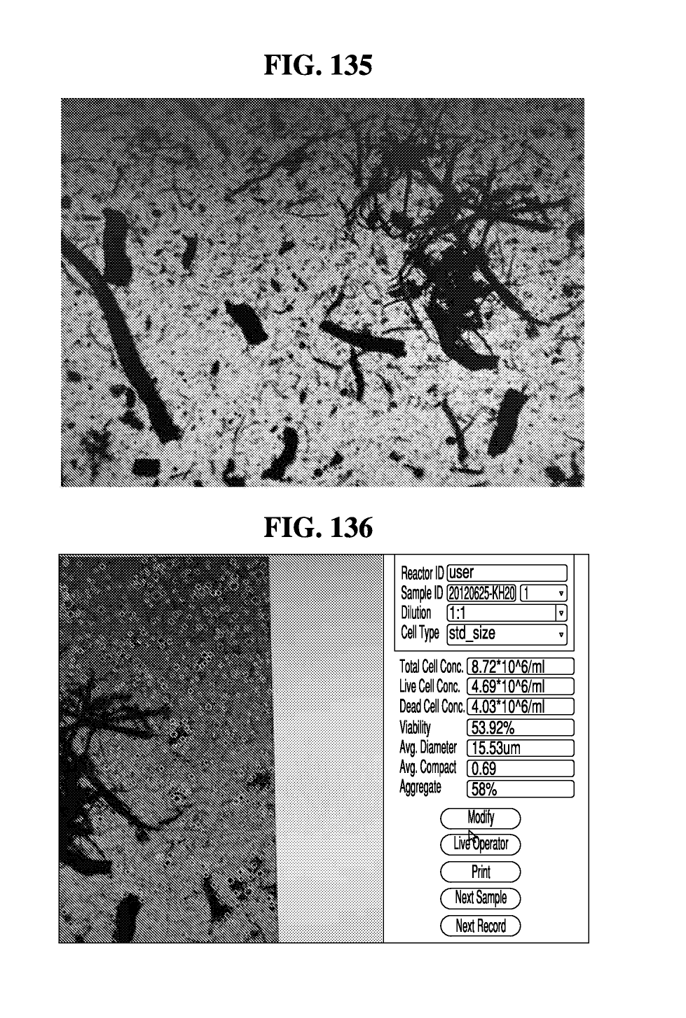

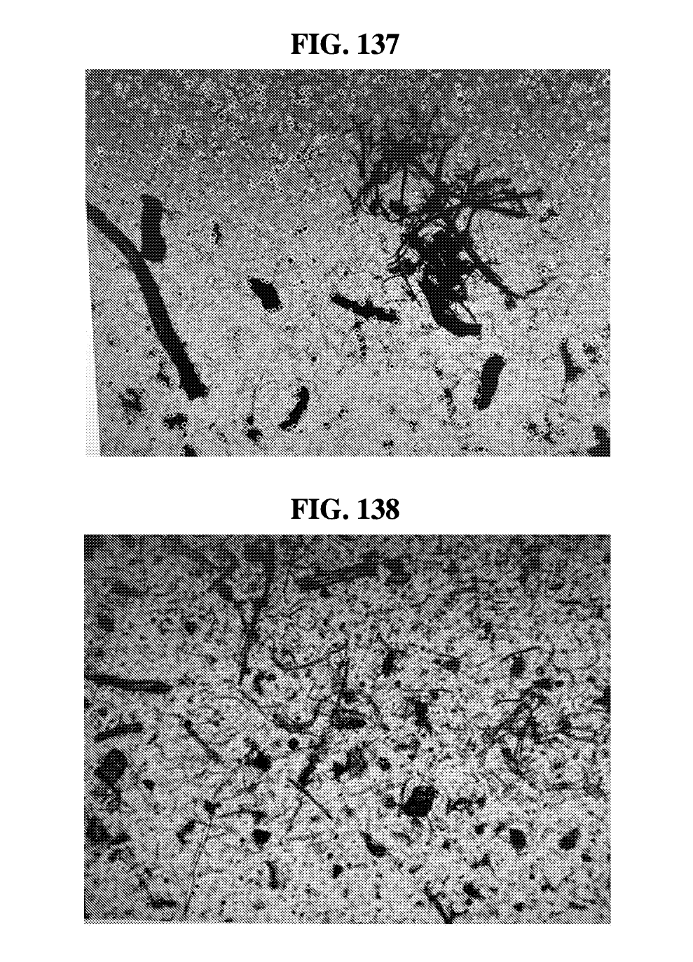

FIGS. 135, 136 and 13—KH205 medium sample number 4. Without Tryptophan.

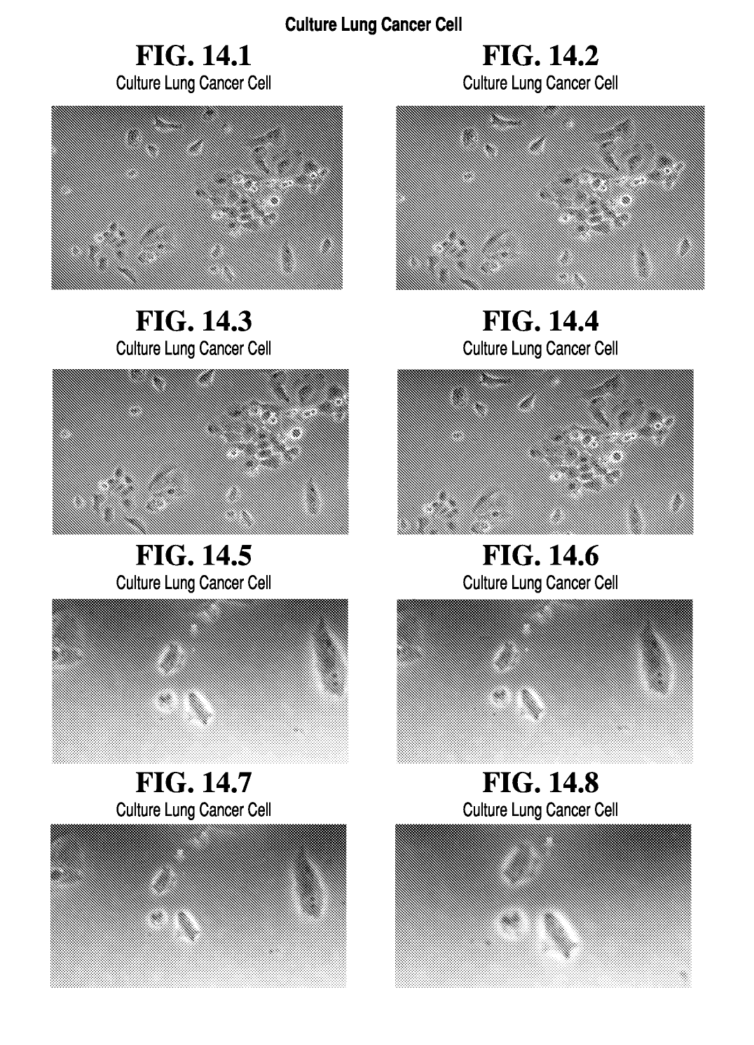

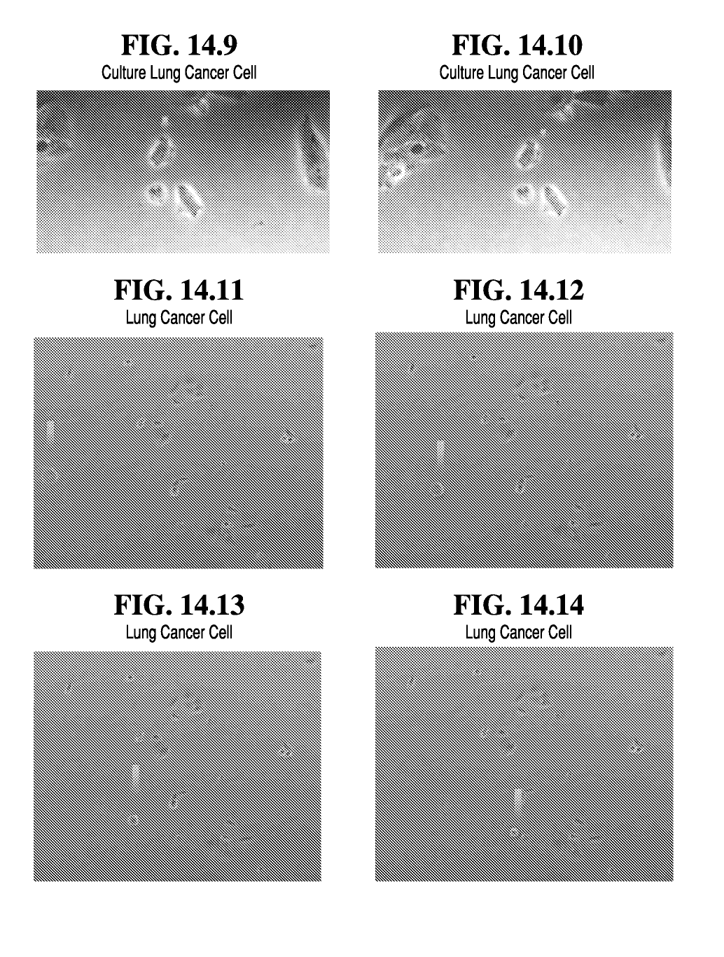

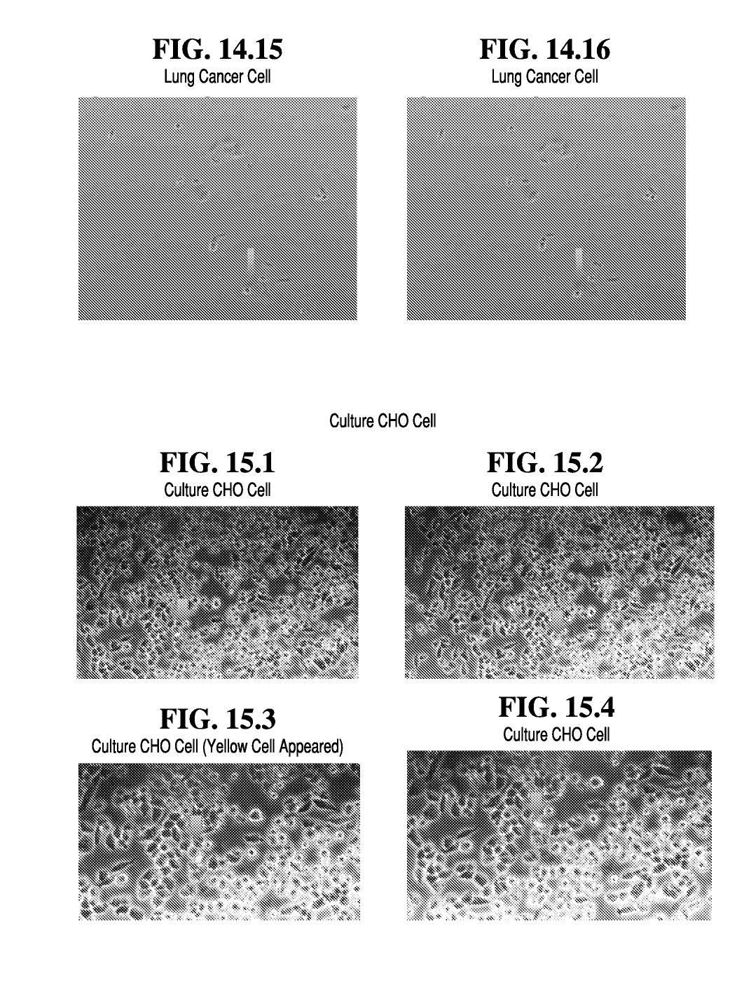

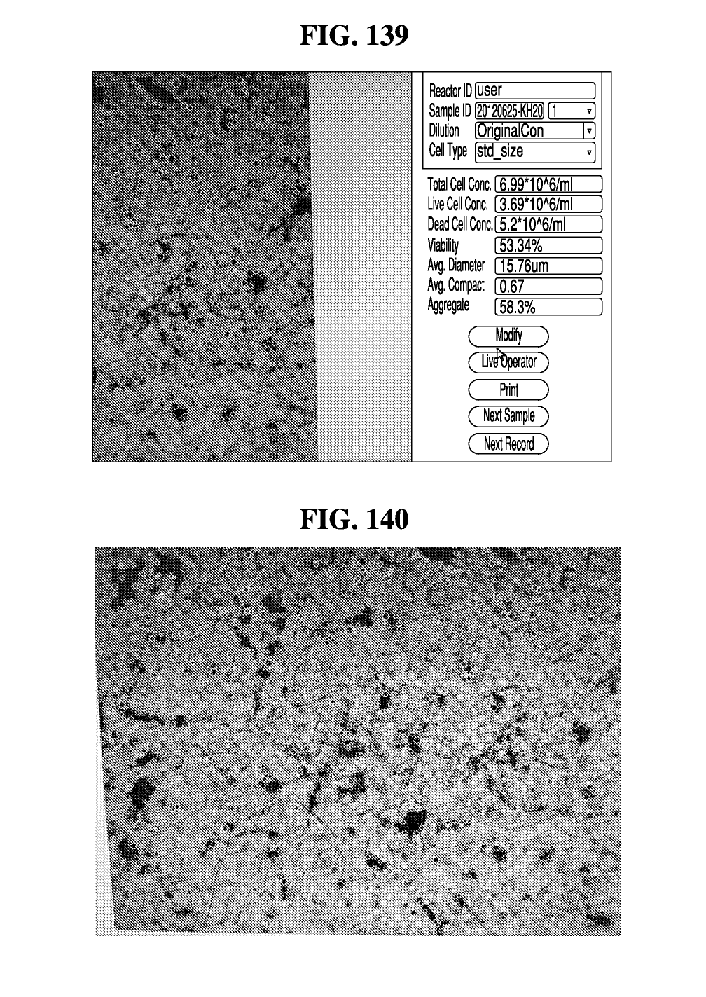

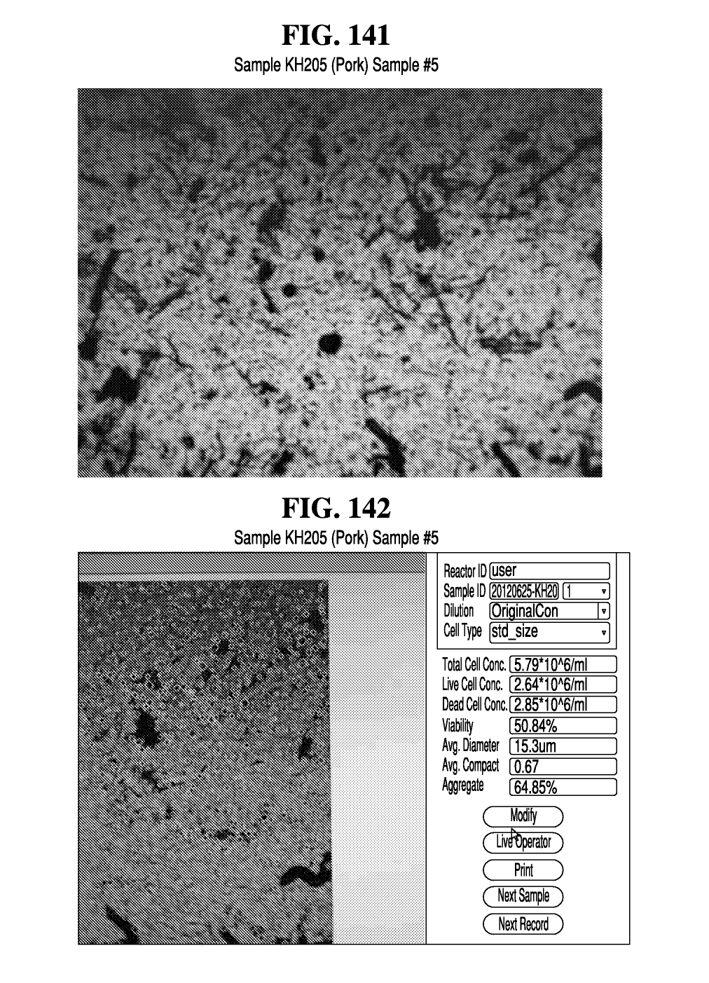

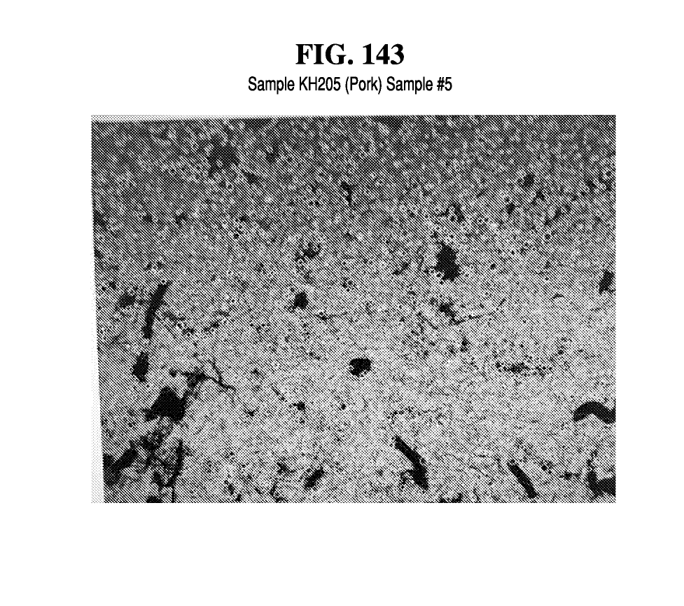

FIGS. 138, 139 and 14—KH205 medium sample number 5. Without Tryptophan.

FIGS. 141, 142 and 14—KH 206 medium contains 37 g of paste of Beef with 750 mL of WFI. Sample number 1, Tryptophan is added as a stabilizer.

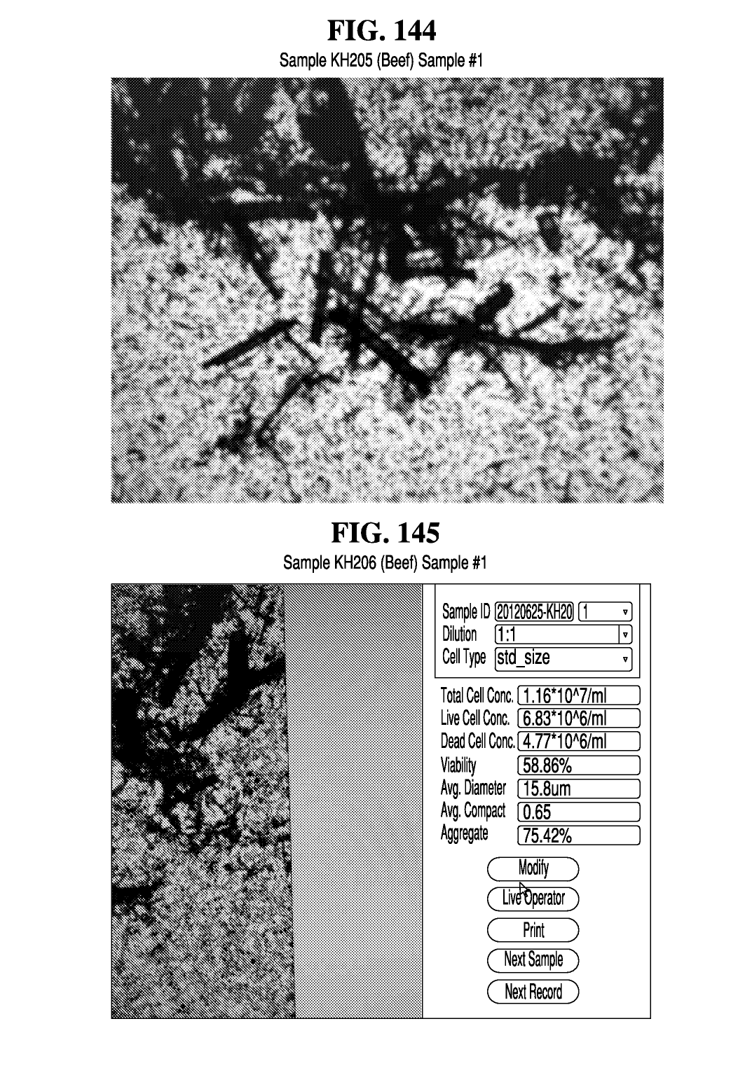

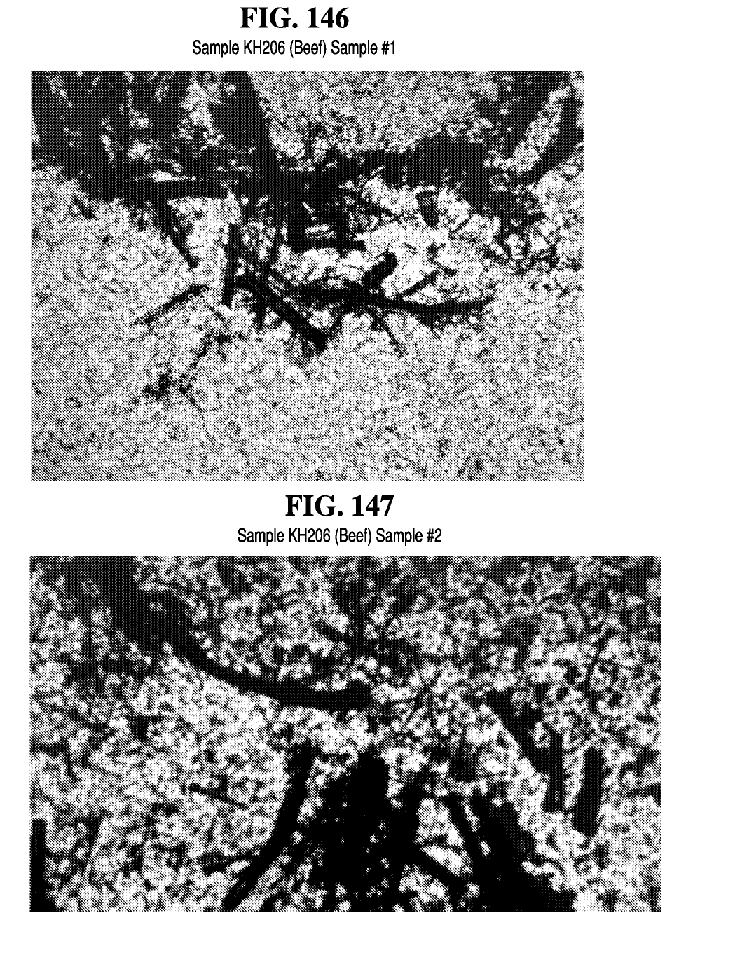

FIGS. 144, 145 and 14—KH206 medium sample number 2. Tryptophan is added as a stabilizer.

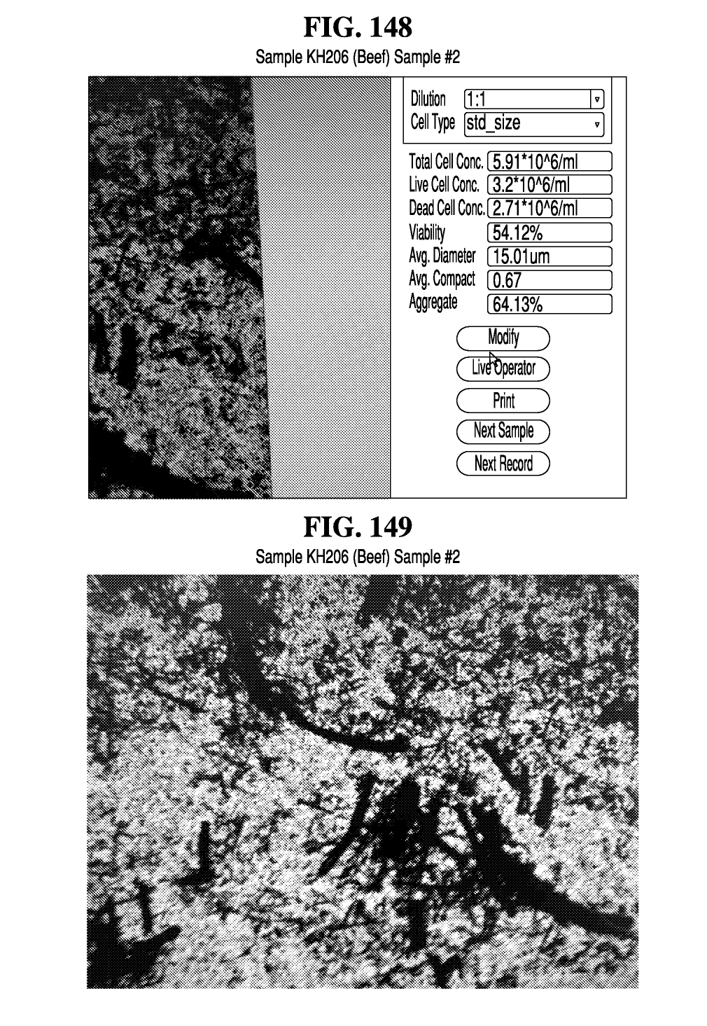

FIGS. 147, 148 and 14—KH206 medium sample number 3. Tryptophan is added as a stabilizer.

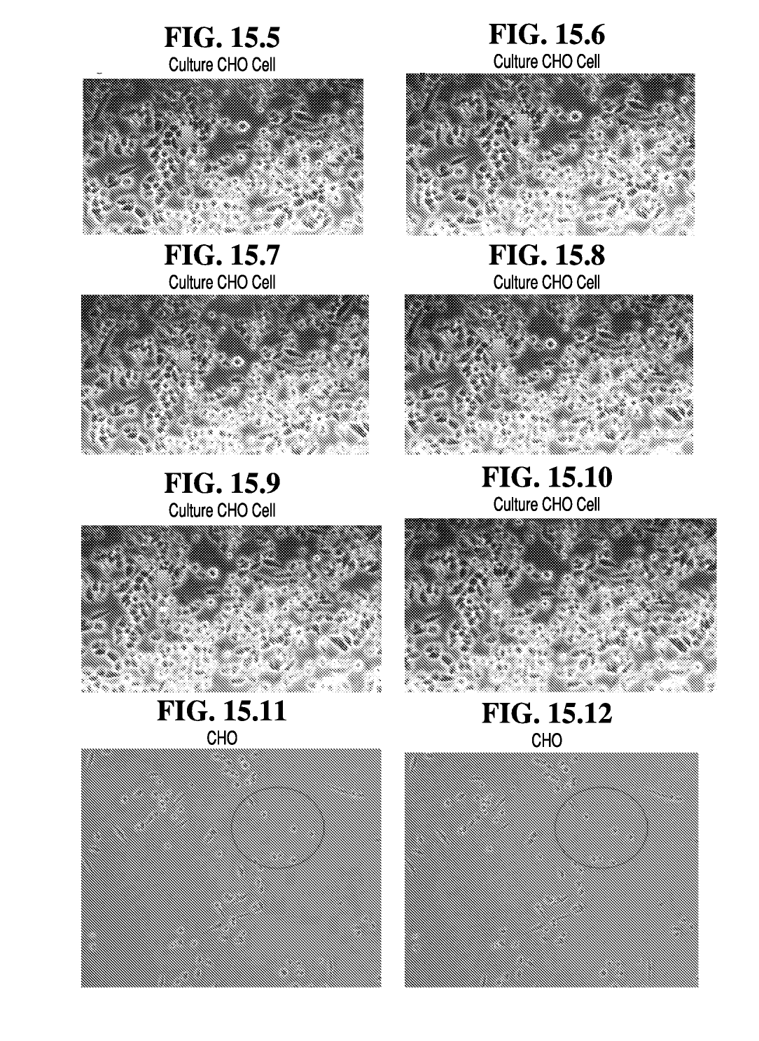

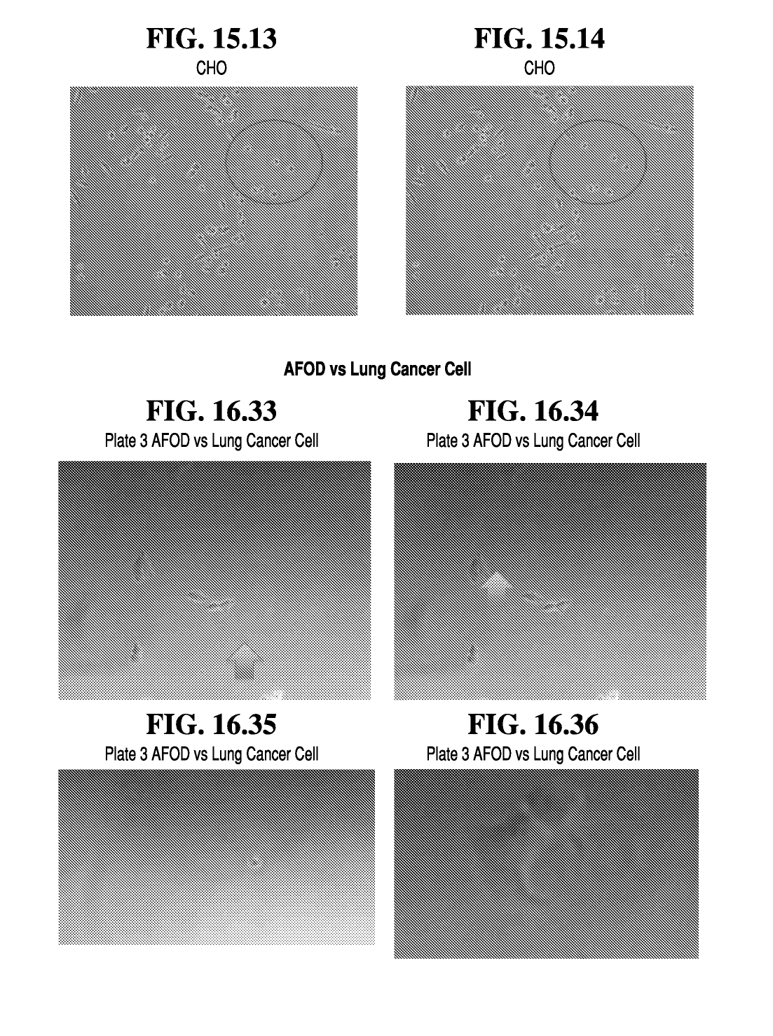

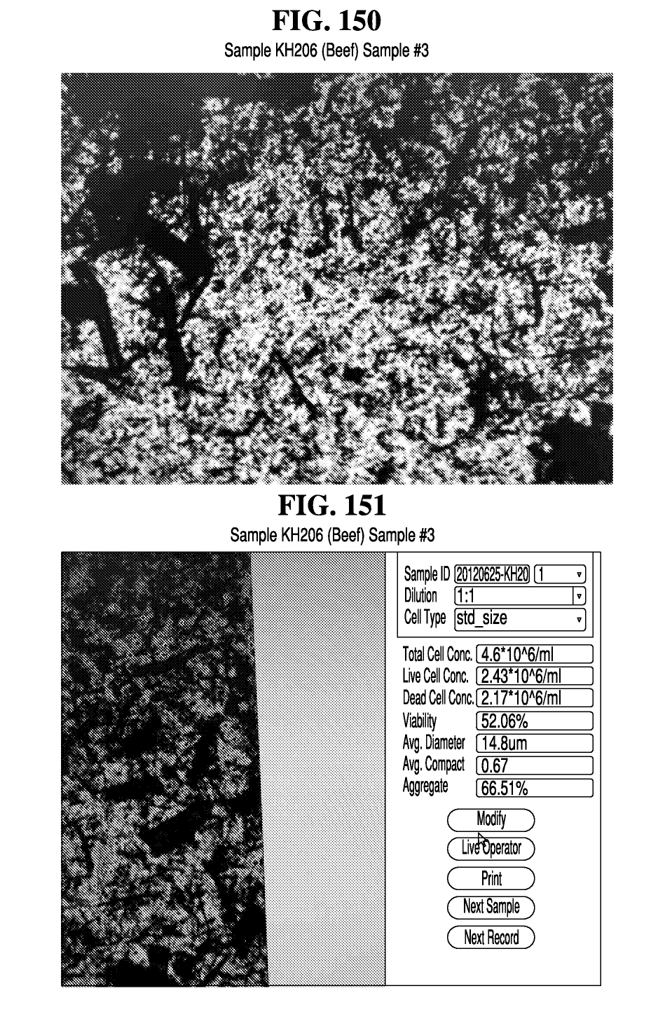

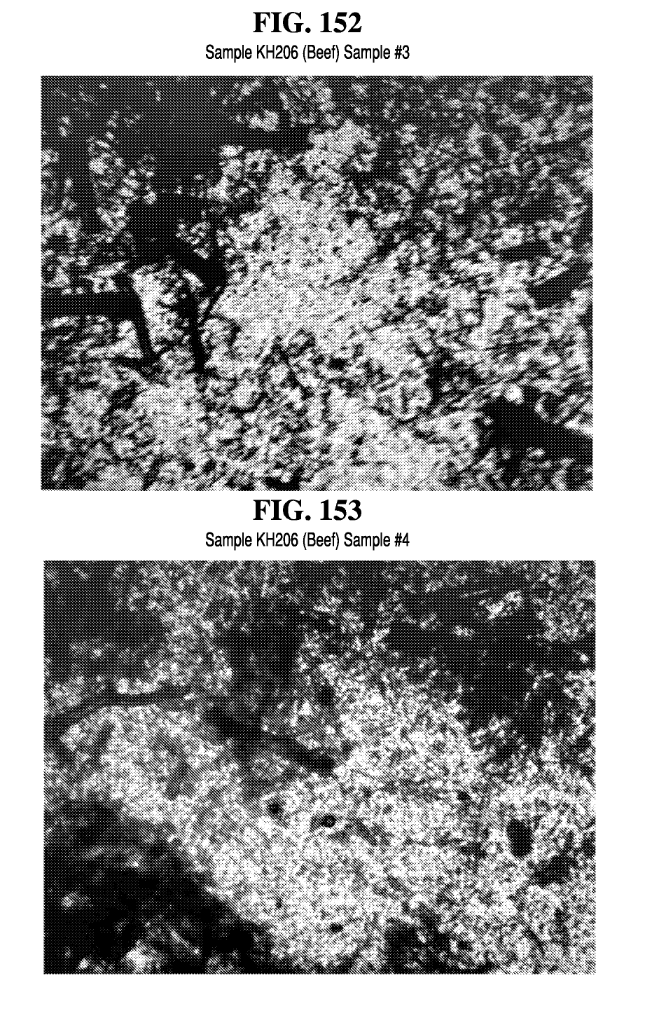

FIGS. 150, 151 and 15—KH206 medium sample number 4. Without Tryptophan.

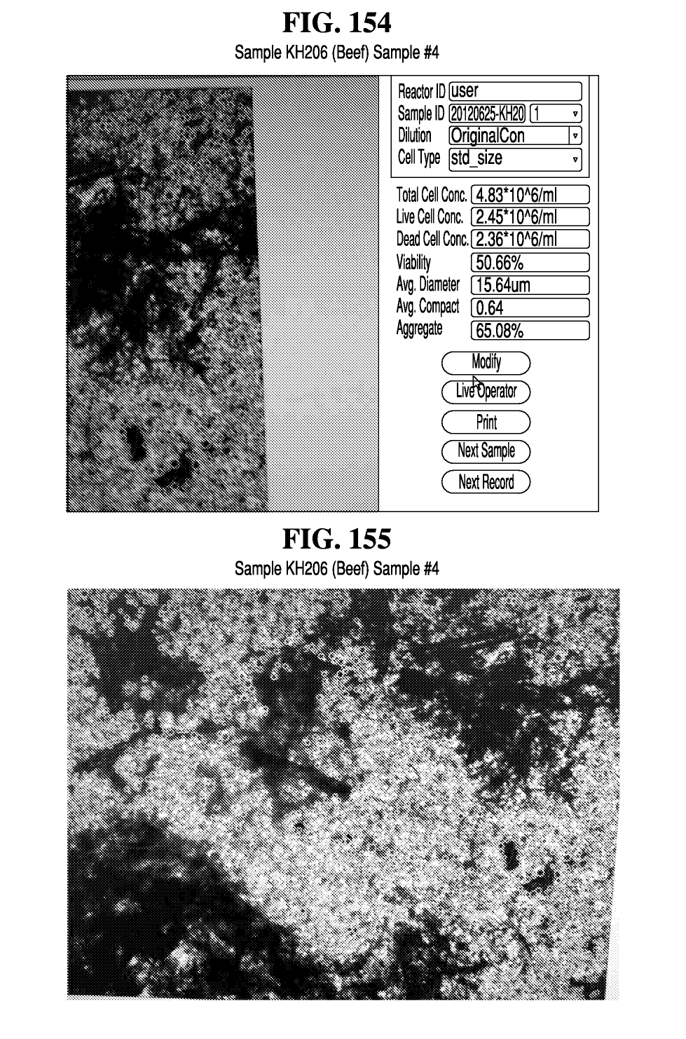

FIGS. 153, 154 and 15—KH206 medium sample number 5. Without Tryptophan.

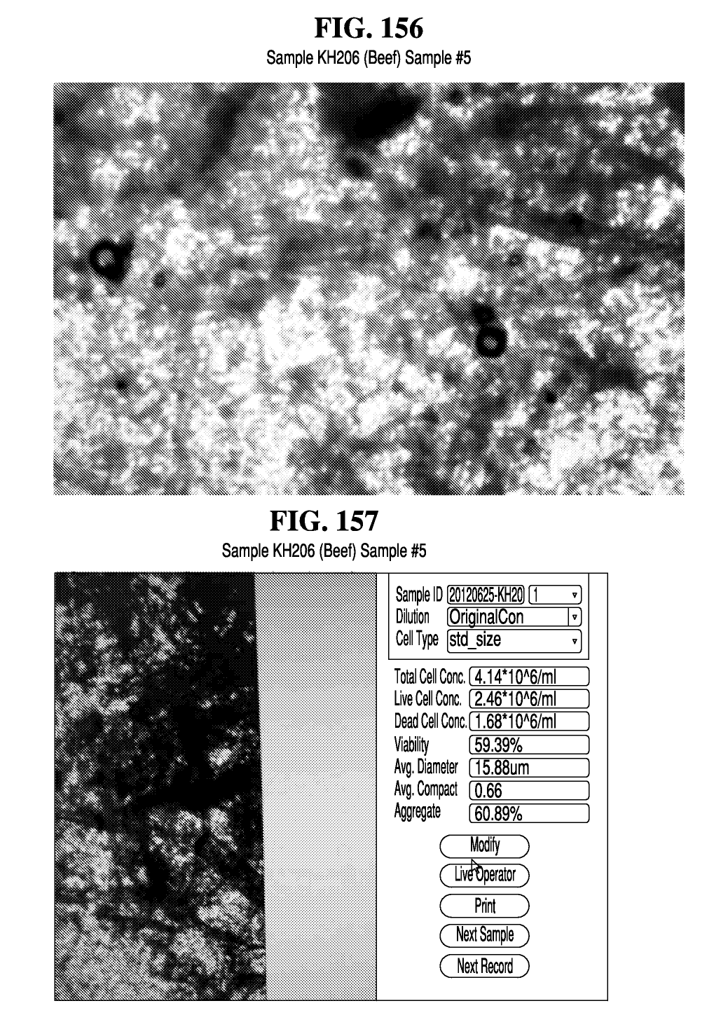

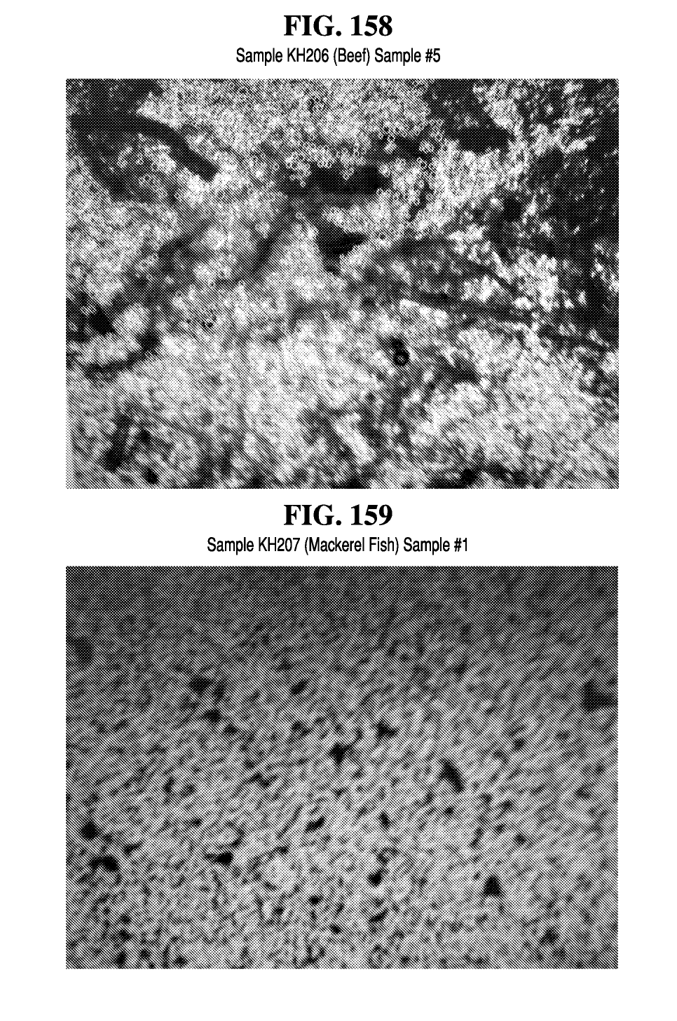

FIGS. 156, 157 and 15—KH 207 medium contains 10.2 g of paste of Mackerel Fish with 200 mL of WFI. Sample number 1, Tryptophan is added as a stabilizer.

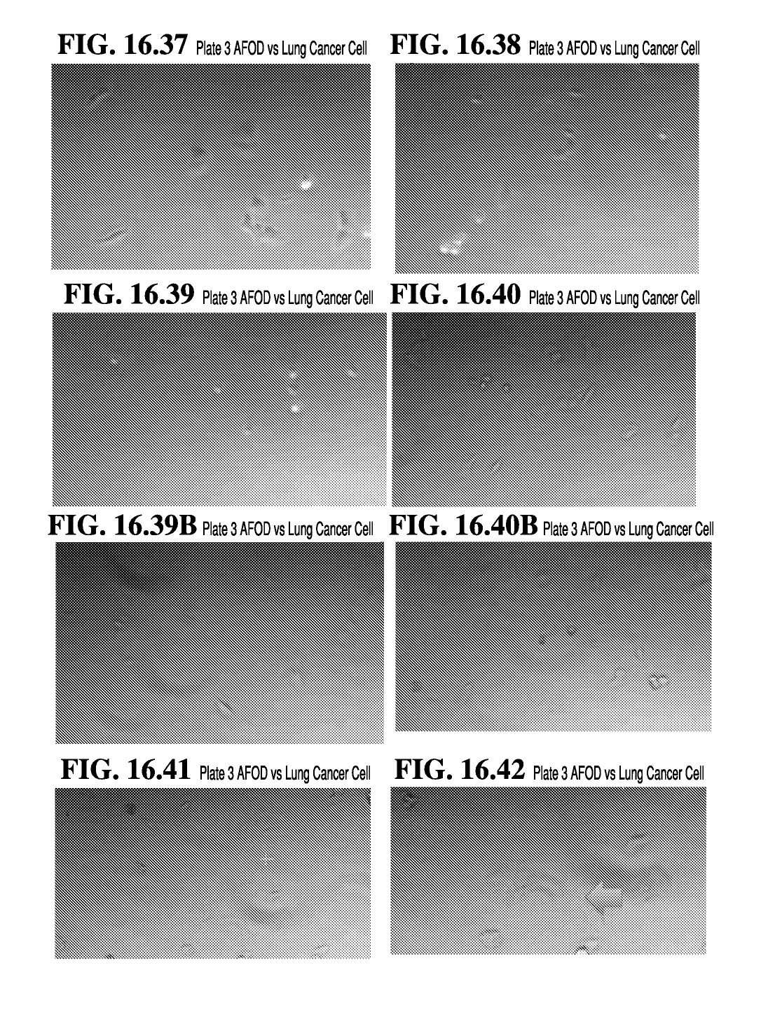

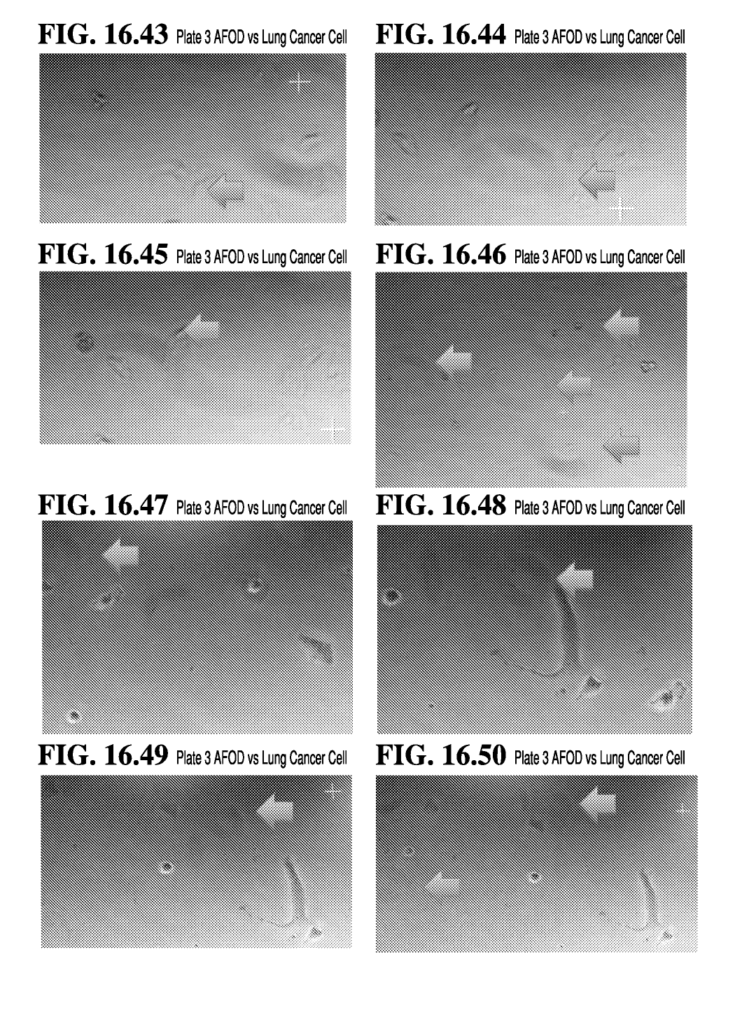

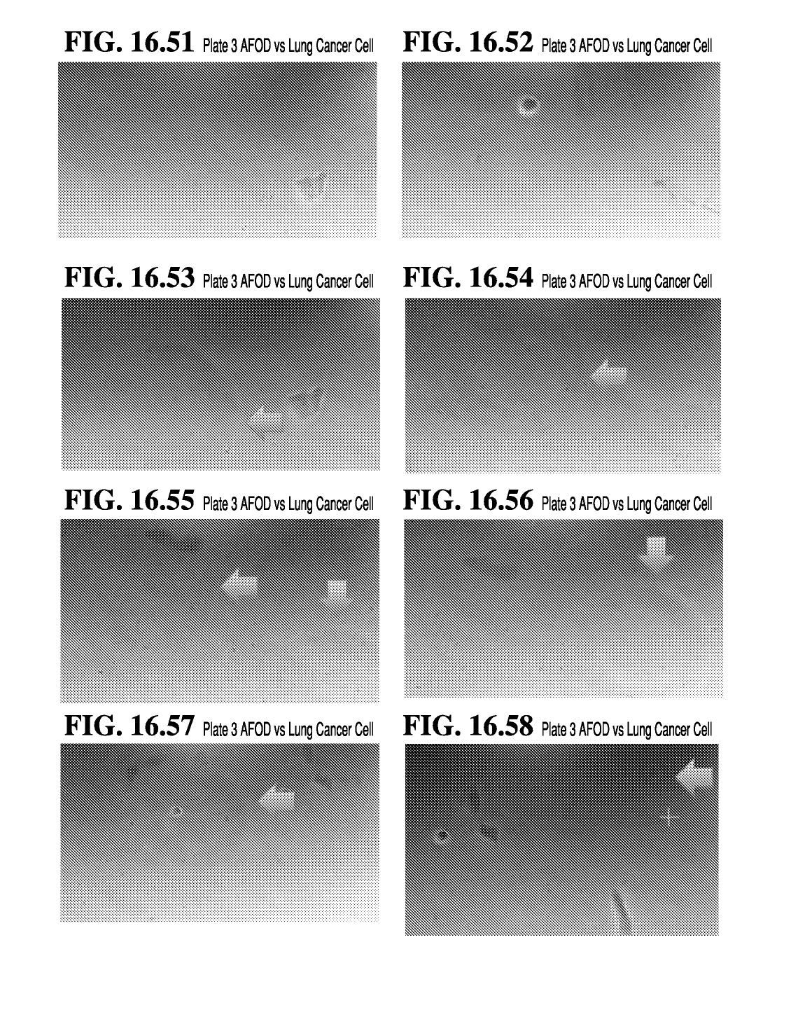

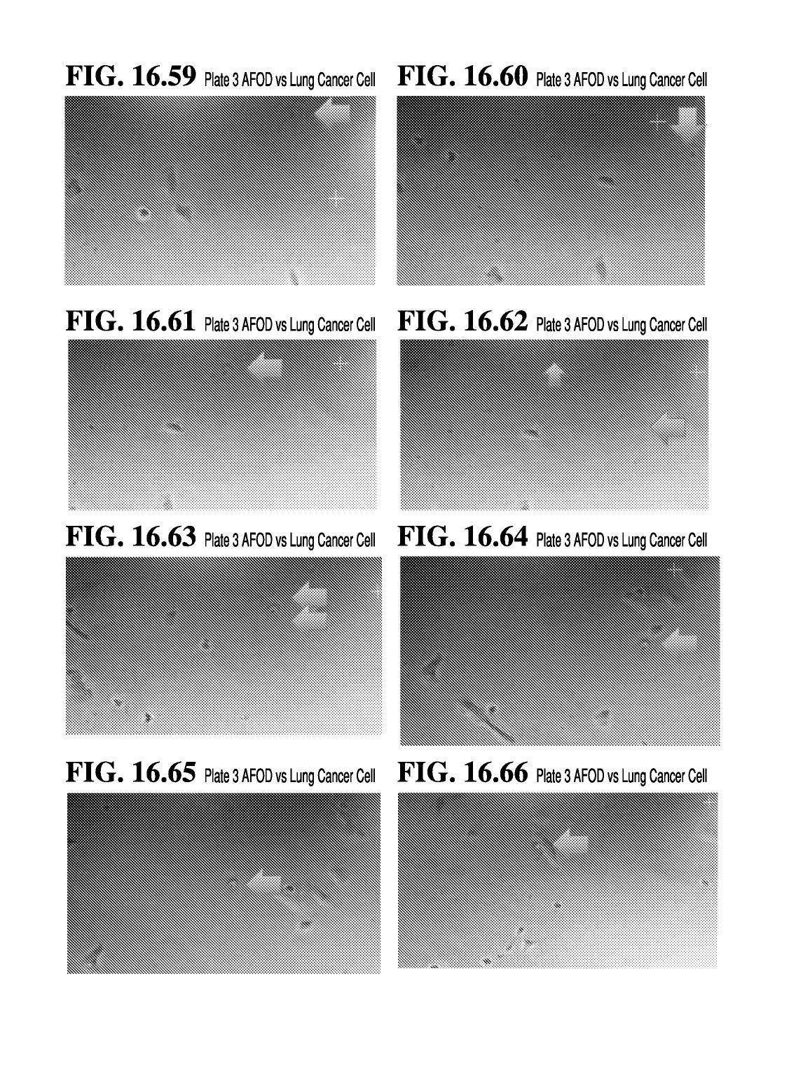

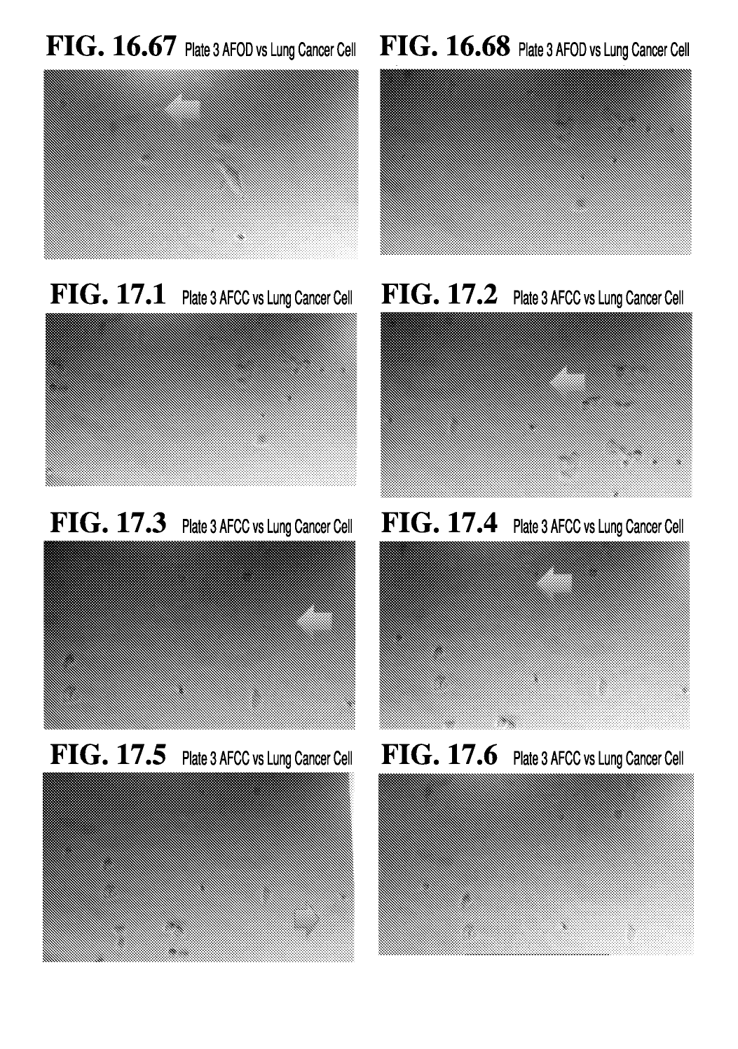

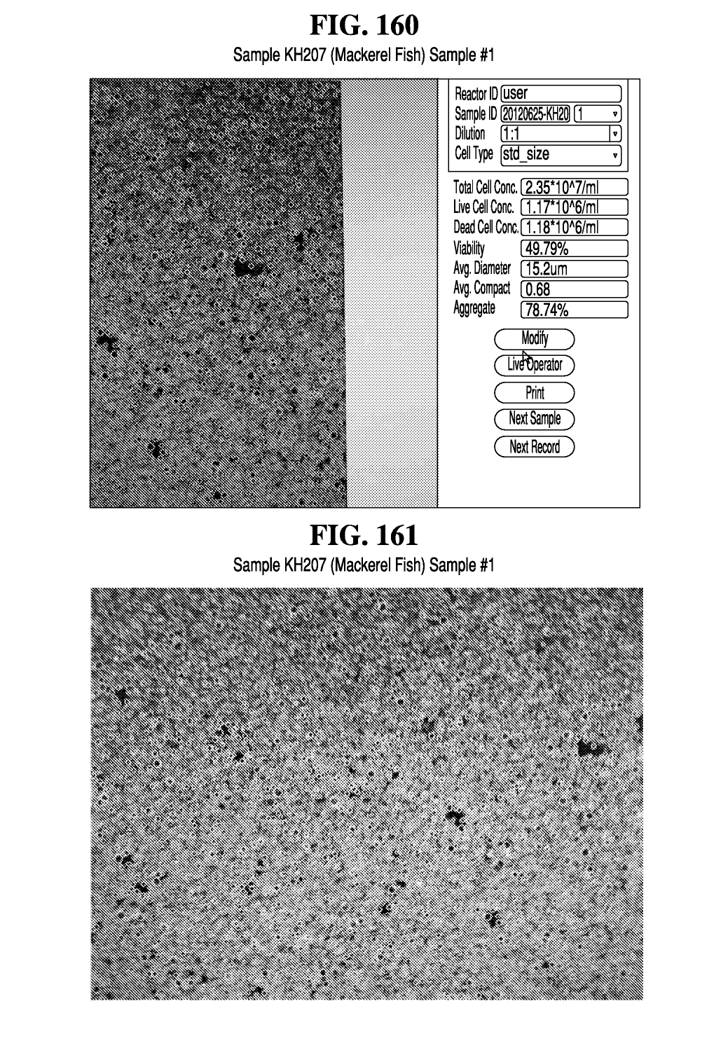

FIGS. 159, 160 and 16—KH207 medium sample number 2. Tryptophan is added as a stabilizer.

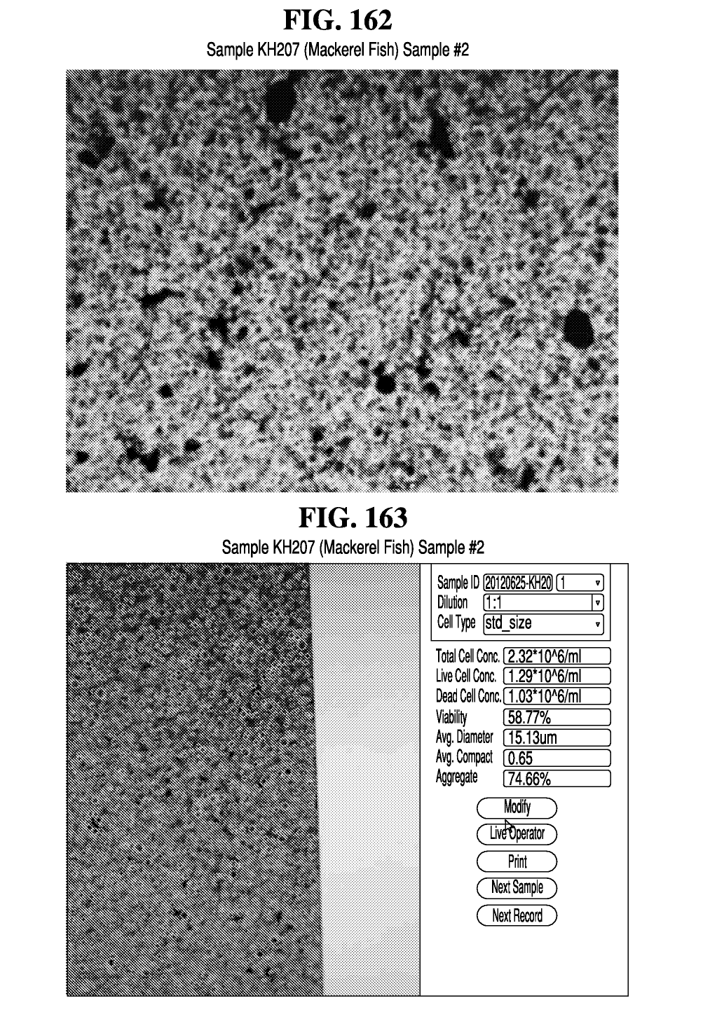

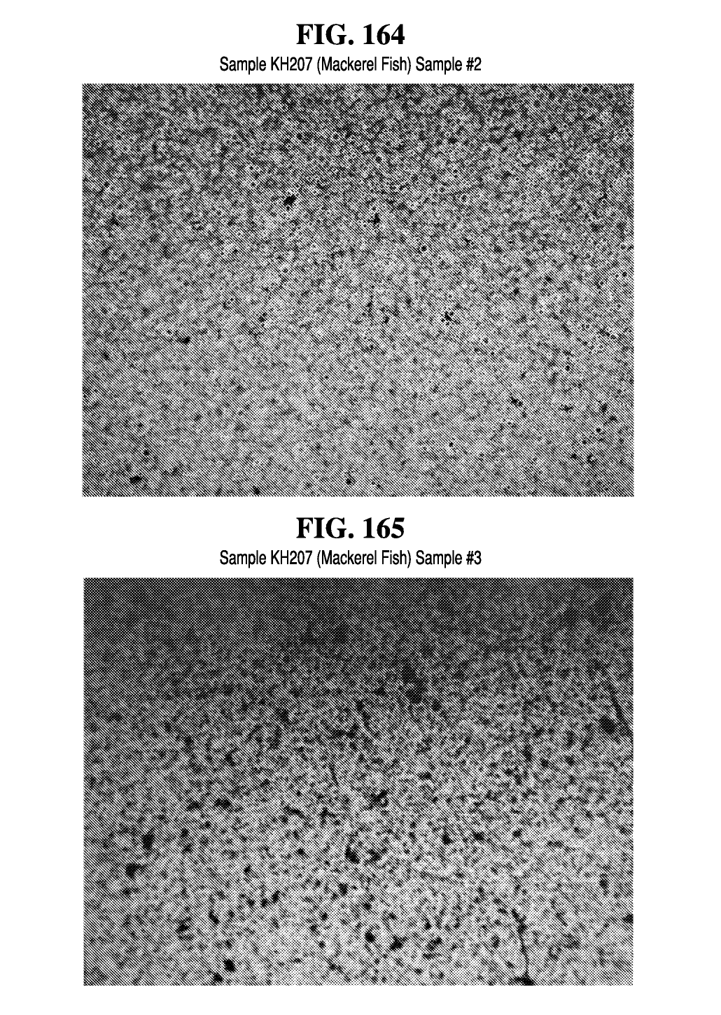

FIGS. 162, 163 and 16—KH207 medium sample number 3. Tryptophan is added as a stabilizer.

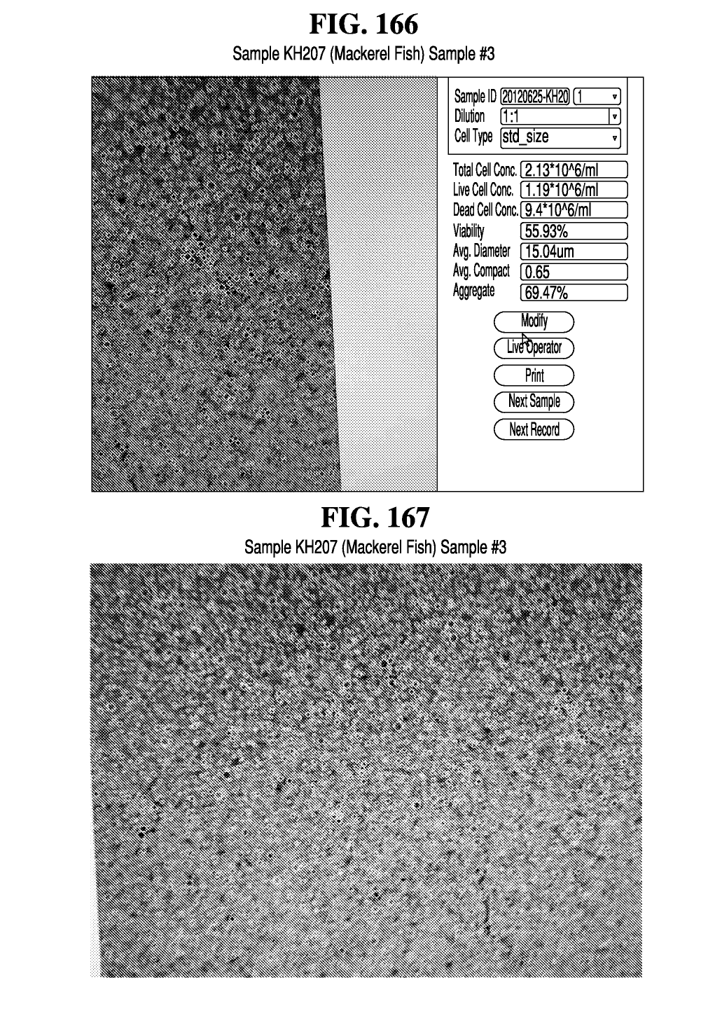

FIGS. 165, 166 and 16—KH207 medium sample number 4. Without Tryptophan.

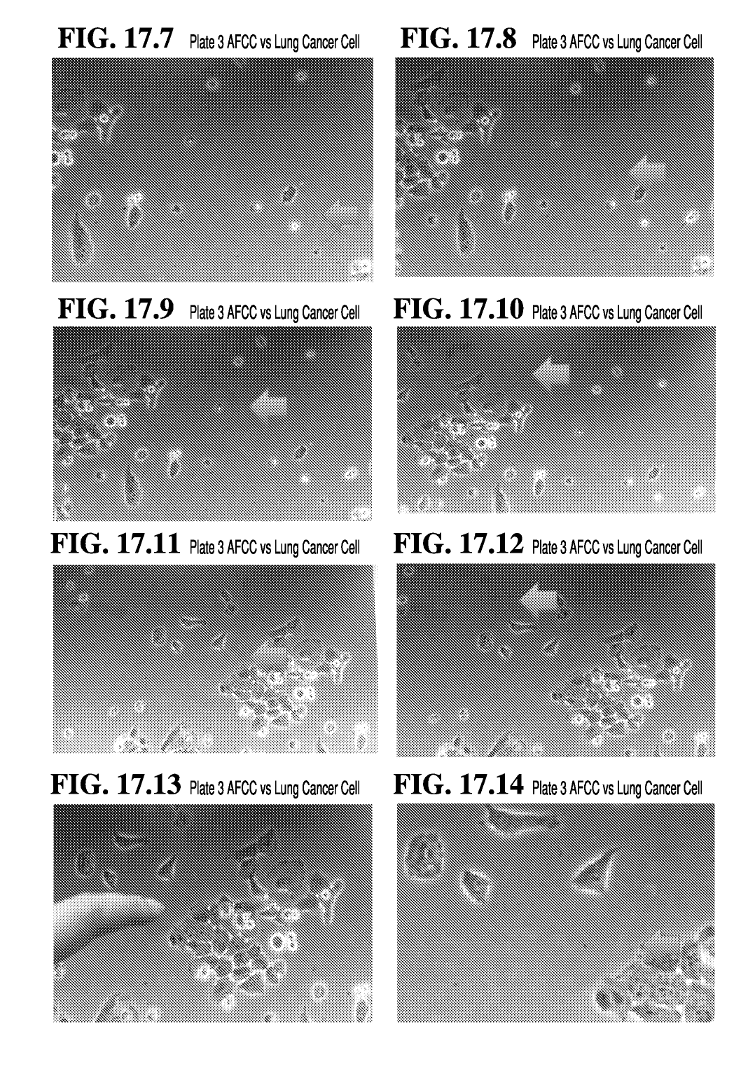

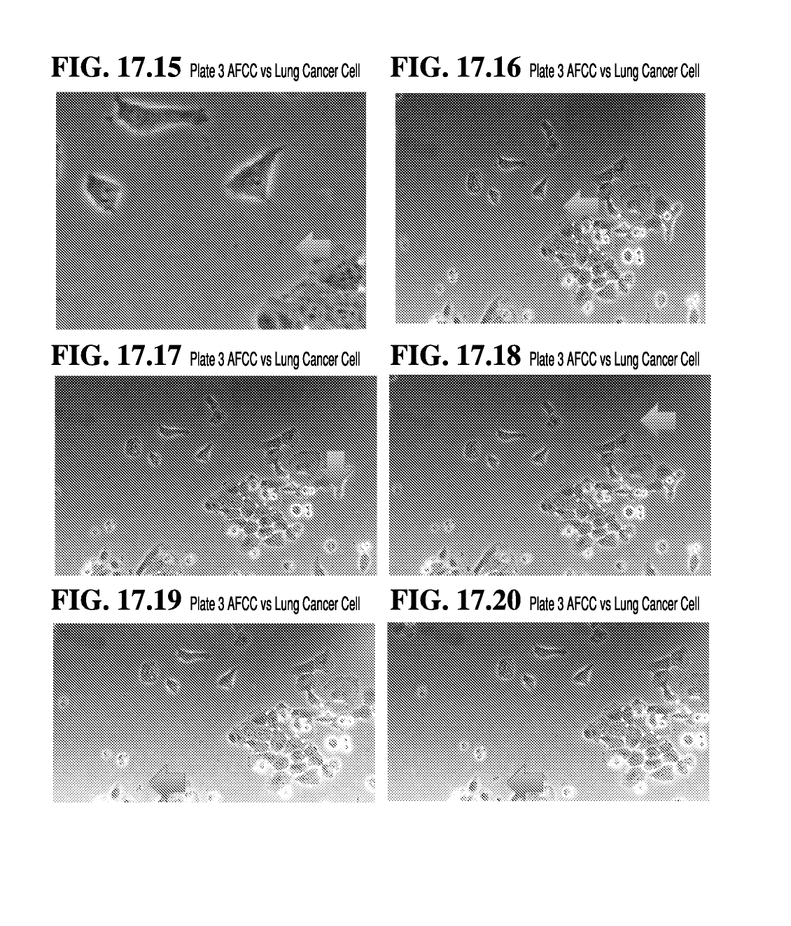

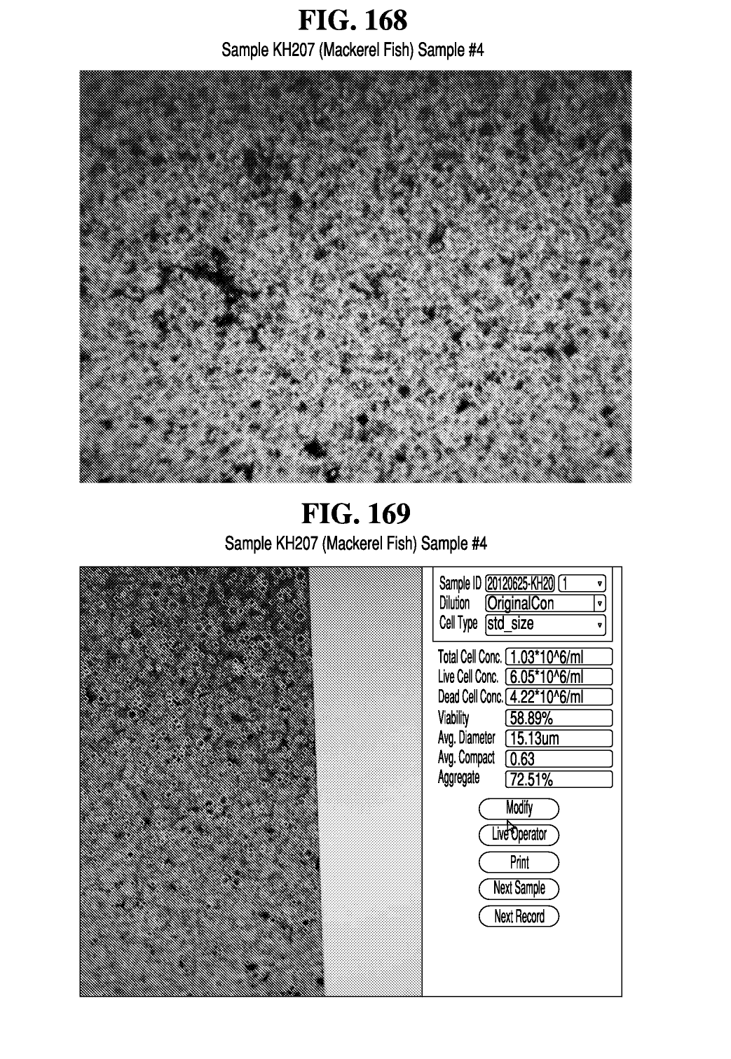

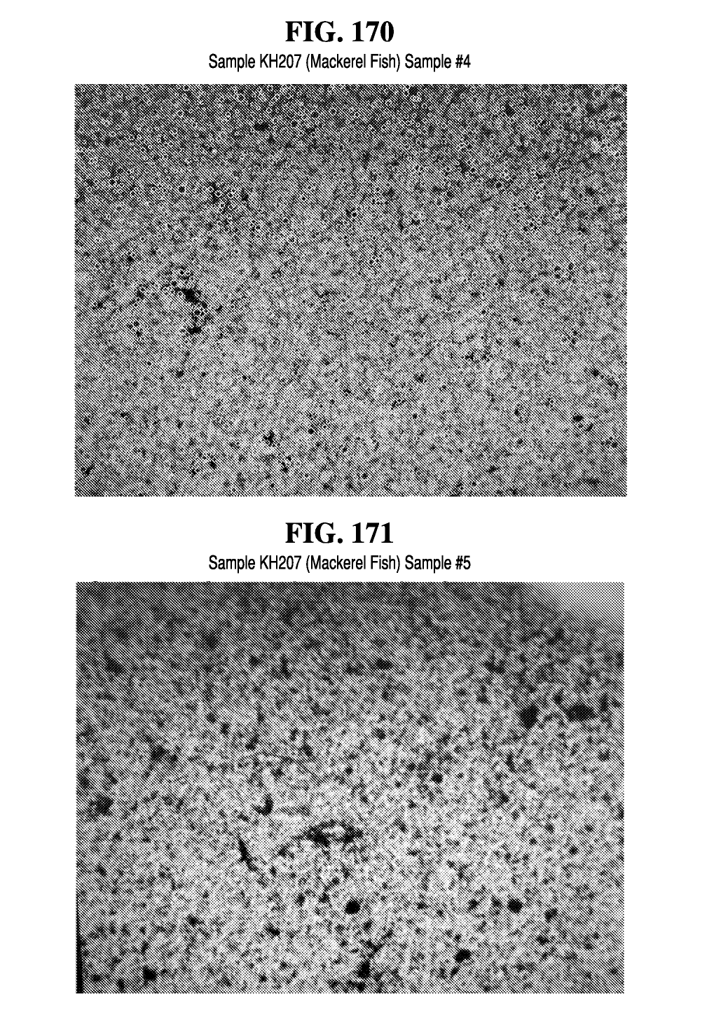

FIGS. 168, 169 and 17—KH207 medium sample number 5. Without Tryptophan.

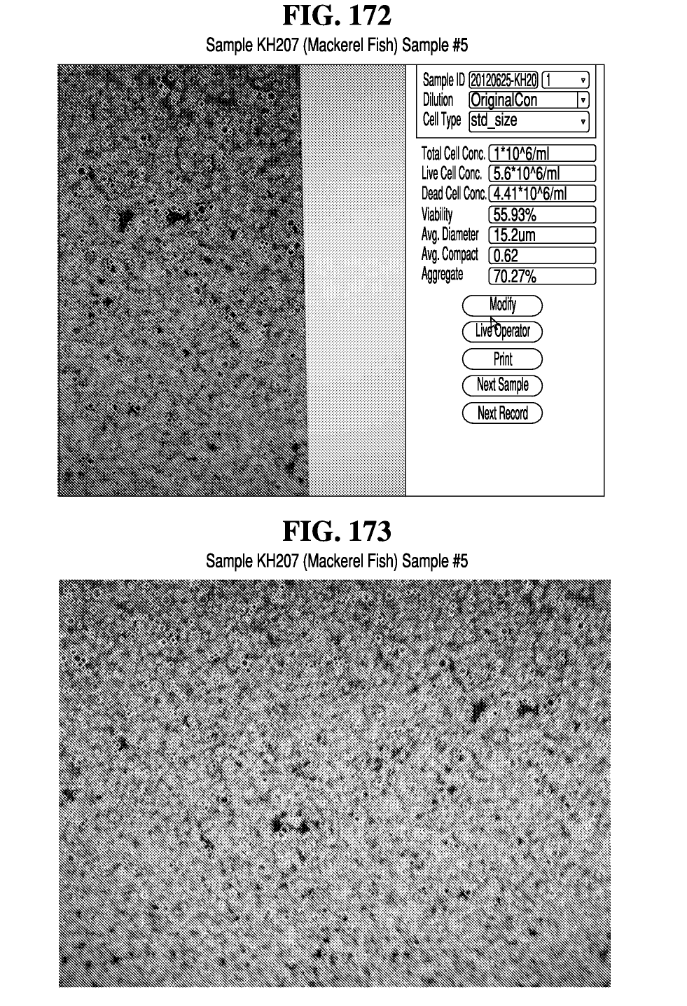

FIGS. 171, 172 and 17—KH 208 medium contains 23.8 g of paste of Chicken with 480 mL of WFI. Sample number 1, Tryptophan is added as a stabilizer.

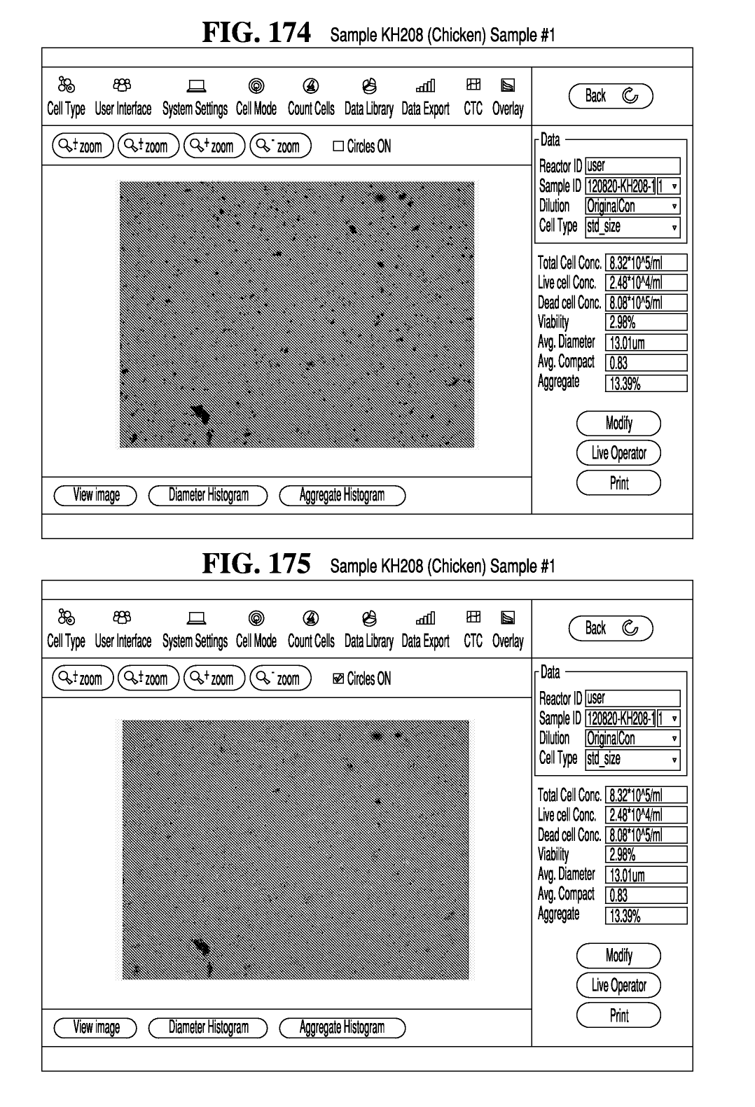

FIGS. 174 and 17—KH 209 medium contains 21.3 g of paste of Shrimp with 420 mL of WFI. Sample number 1, Tryptophan is added as a stabilizer.

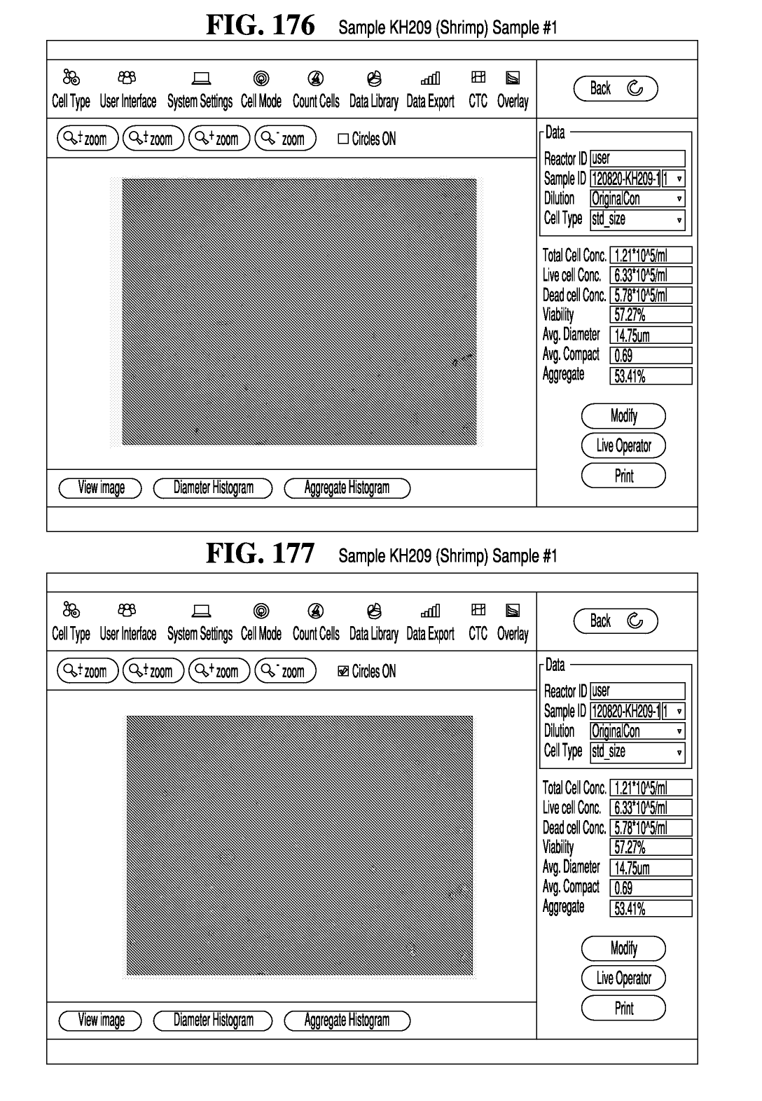

FIGS. 176 and 17—KH 210 medium contains 23.1 g of paste of Egg yoke with 460 mL of WFI. Sample number 1, Tryptophan is added as a stabilizer.

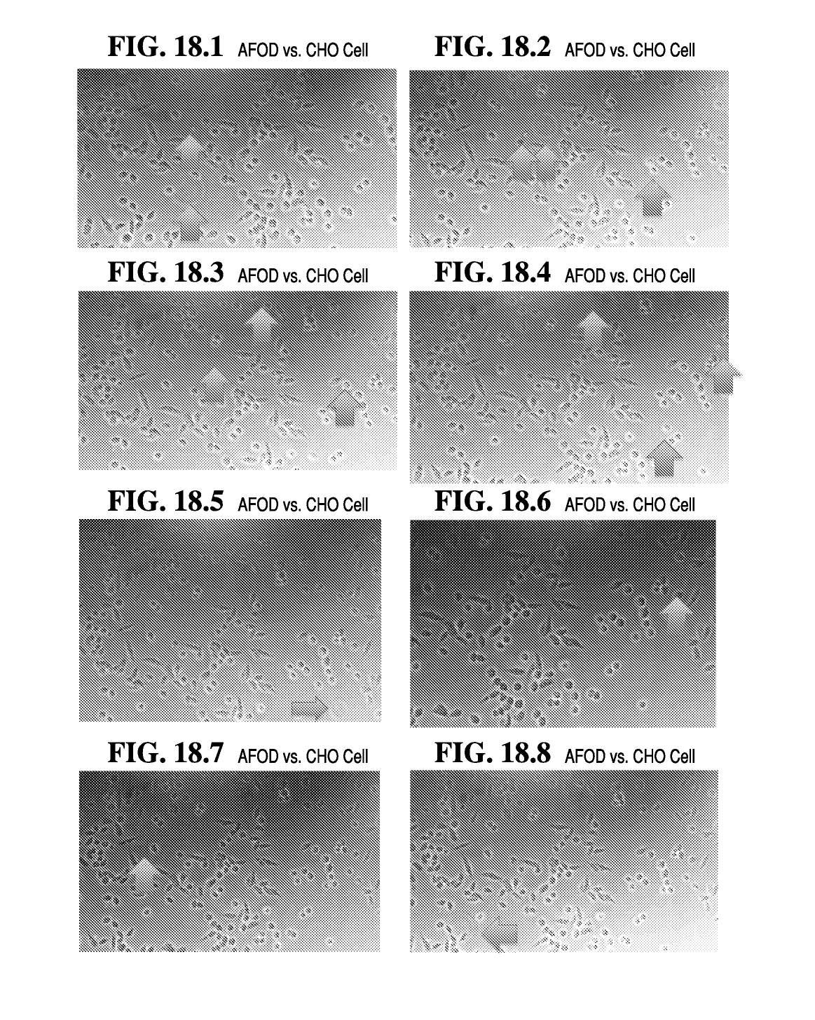

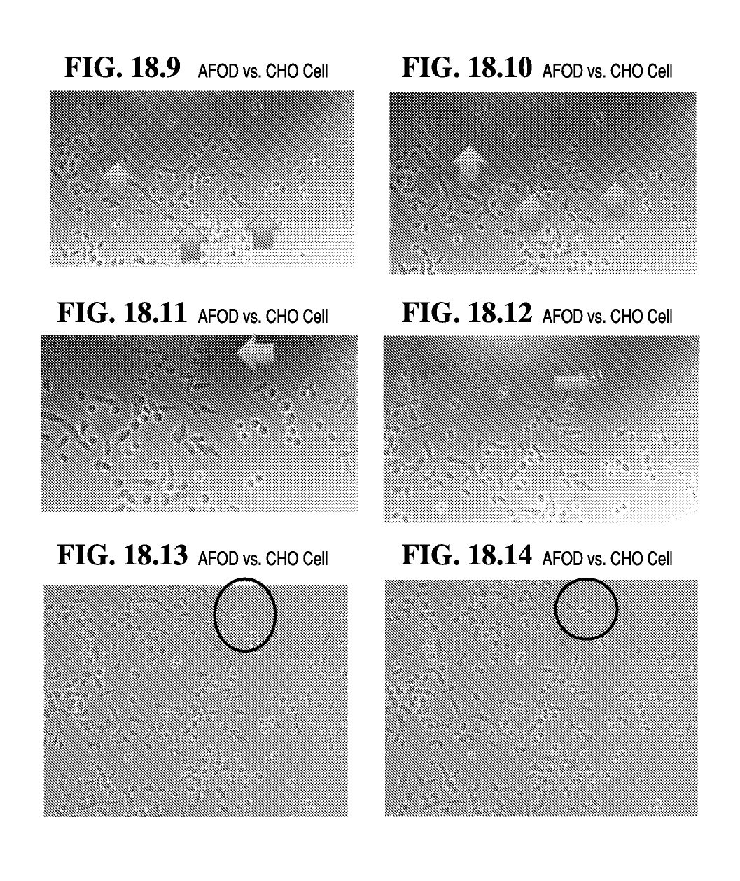

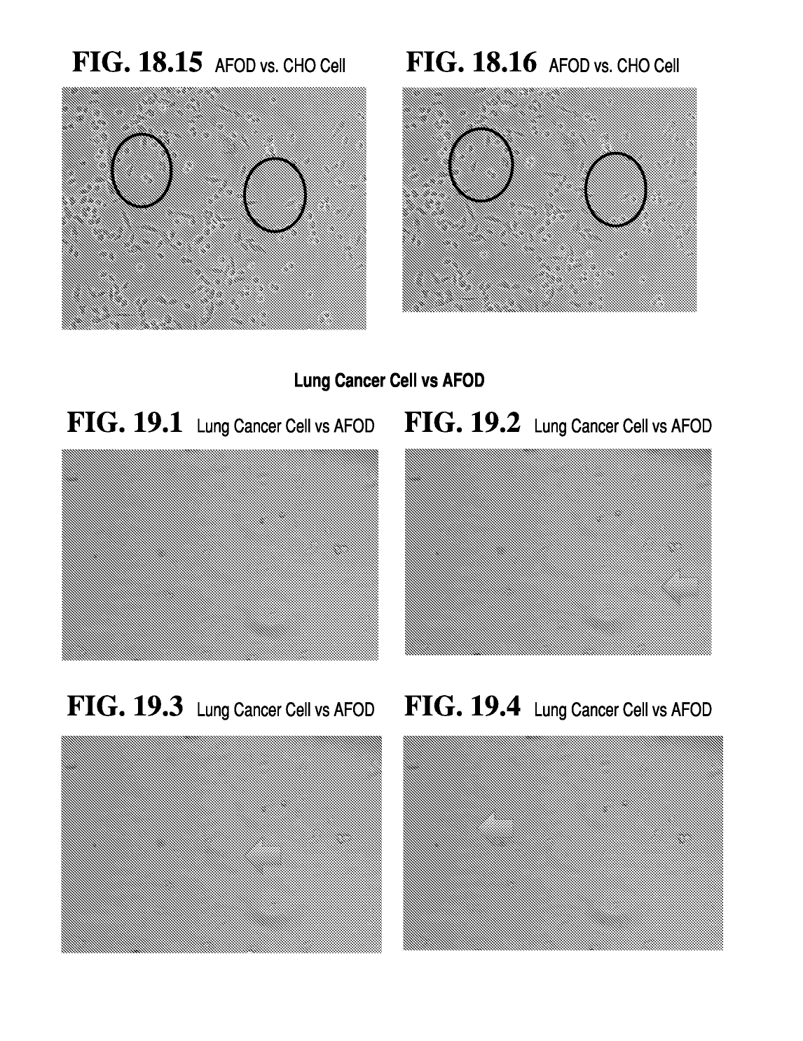

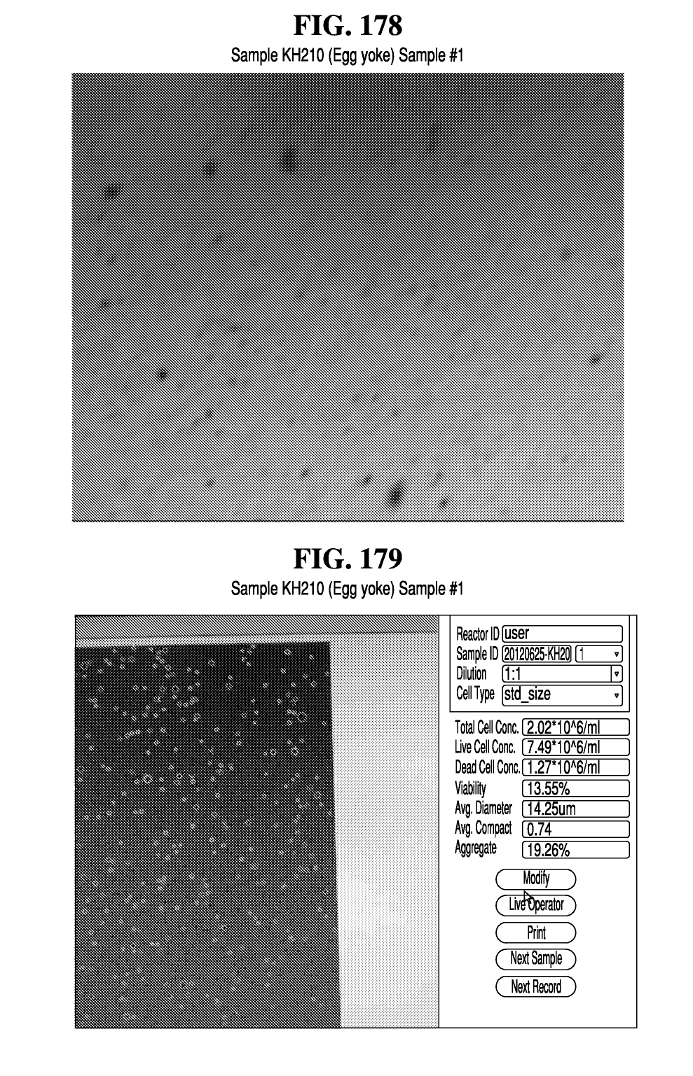

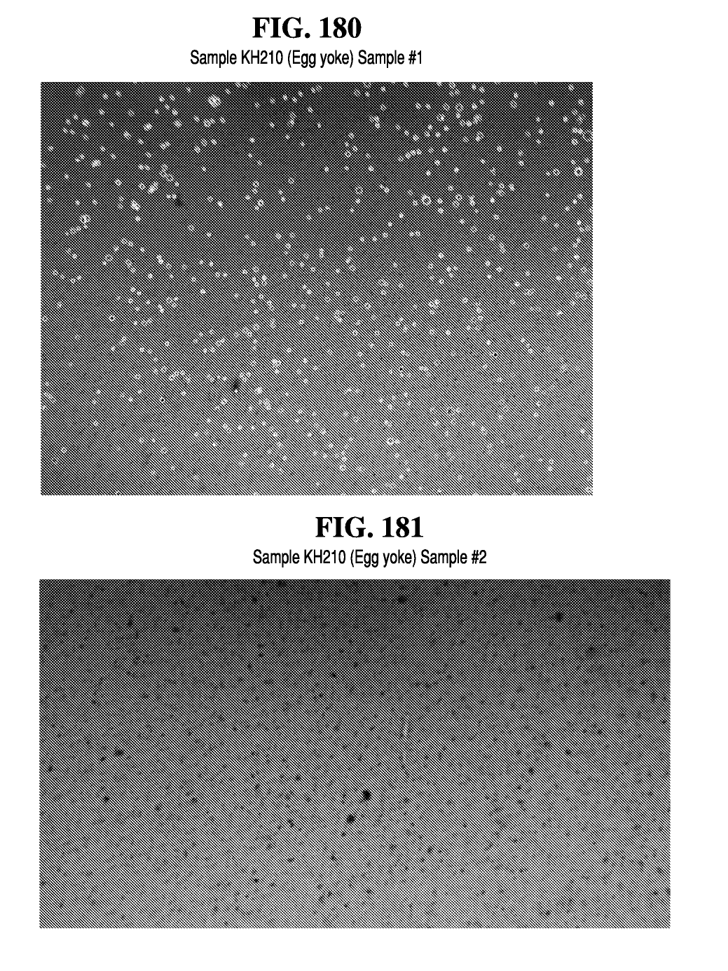

FIGS. 178, 179 and 18—KH210 medium sample number 2. Tryptophan is added as a stabilizer.

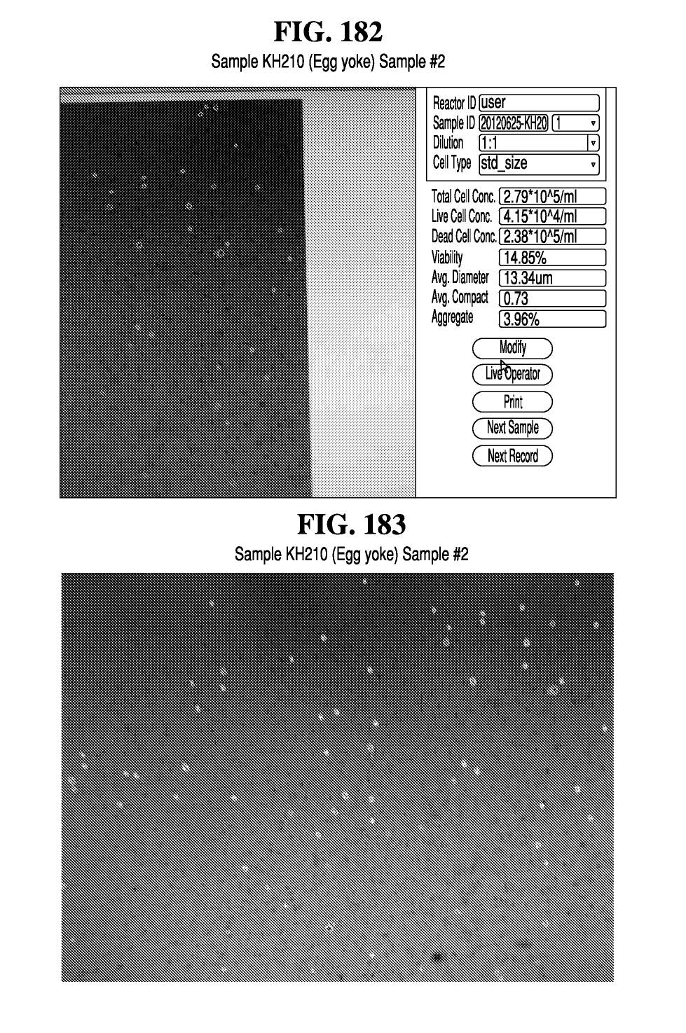

FIGS. 181, 182 and 18—KH210 medium sample number 3. Tryptophan is added as a stabilizer.

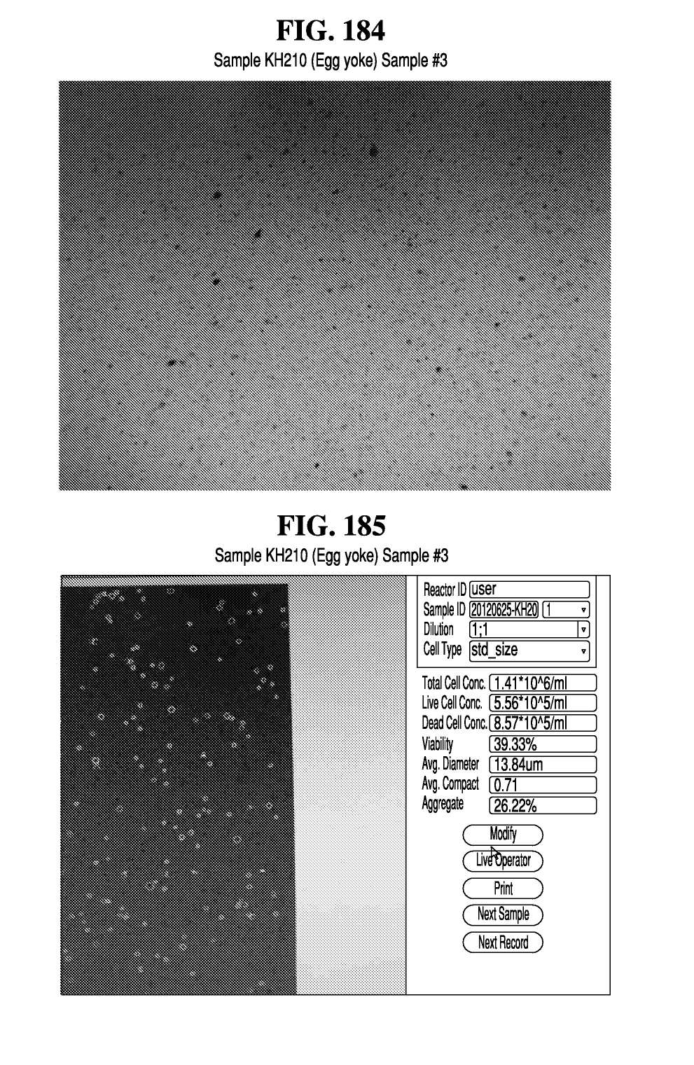

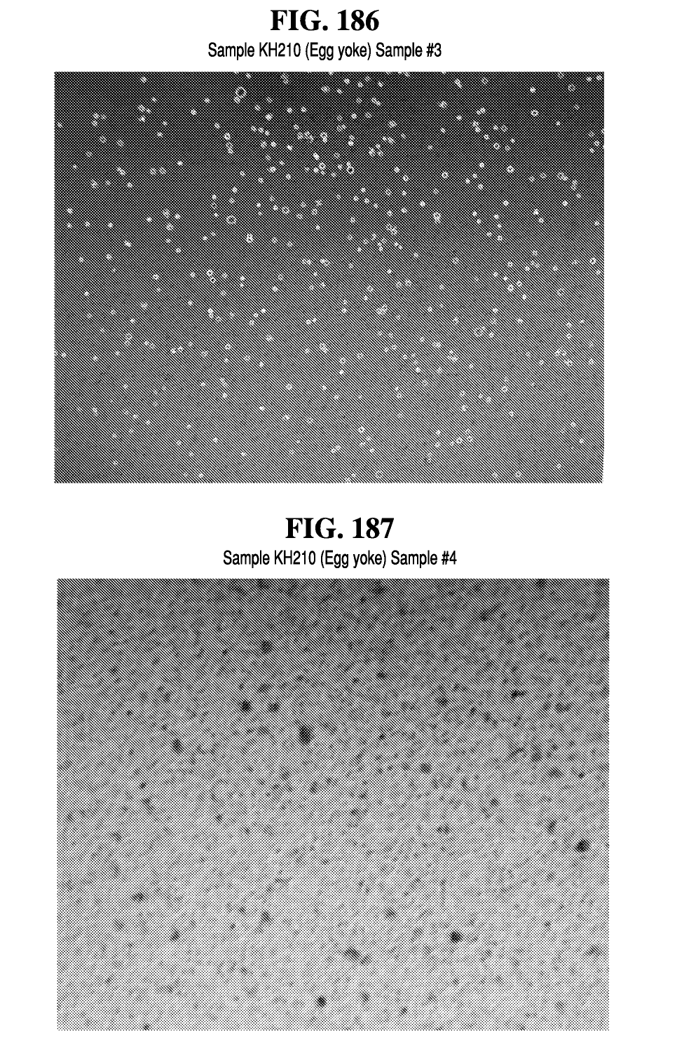

FIGS. 184, 185 and 18—KH210 medium sample number 4. Without Tryptophan.

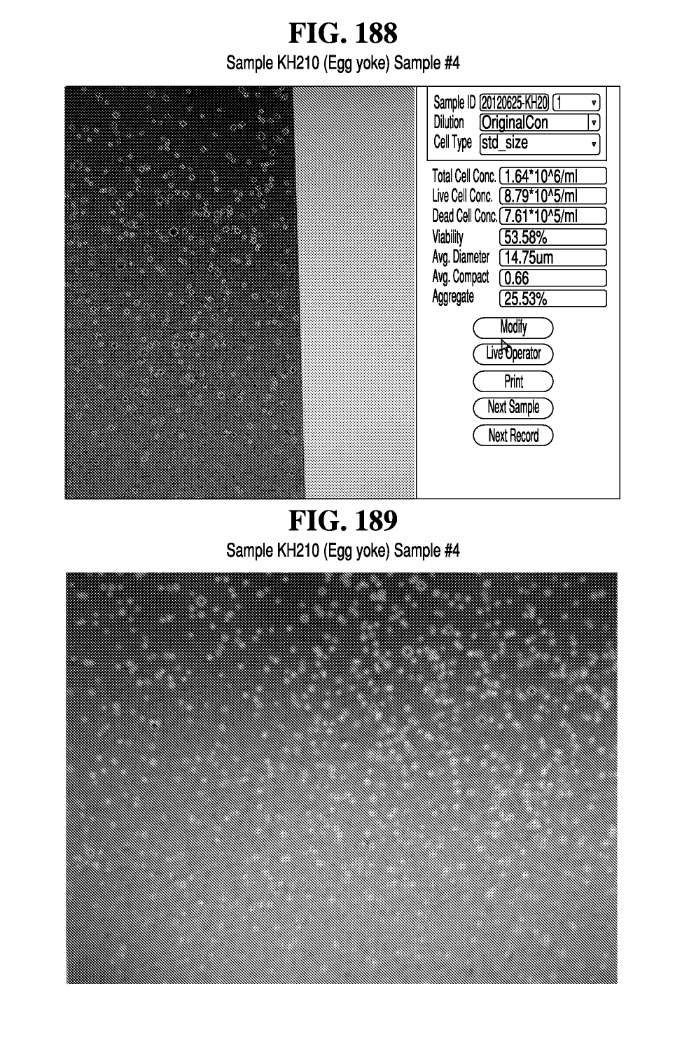

FIGS. 187, 188 and 18—KH210 medium sample number 5. Without Tryptophan.

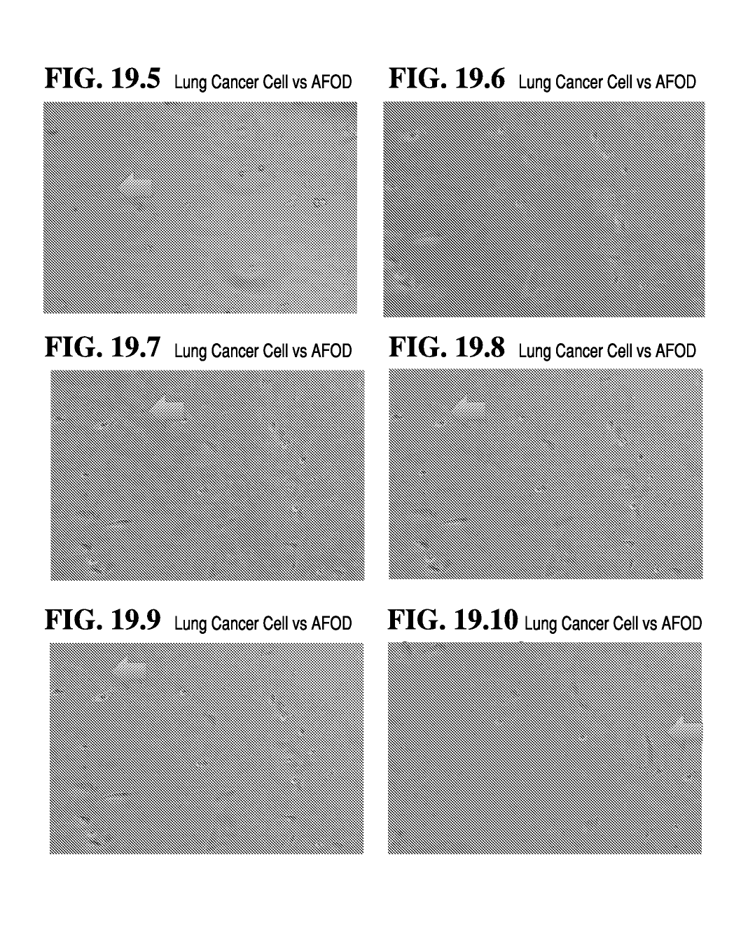

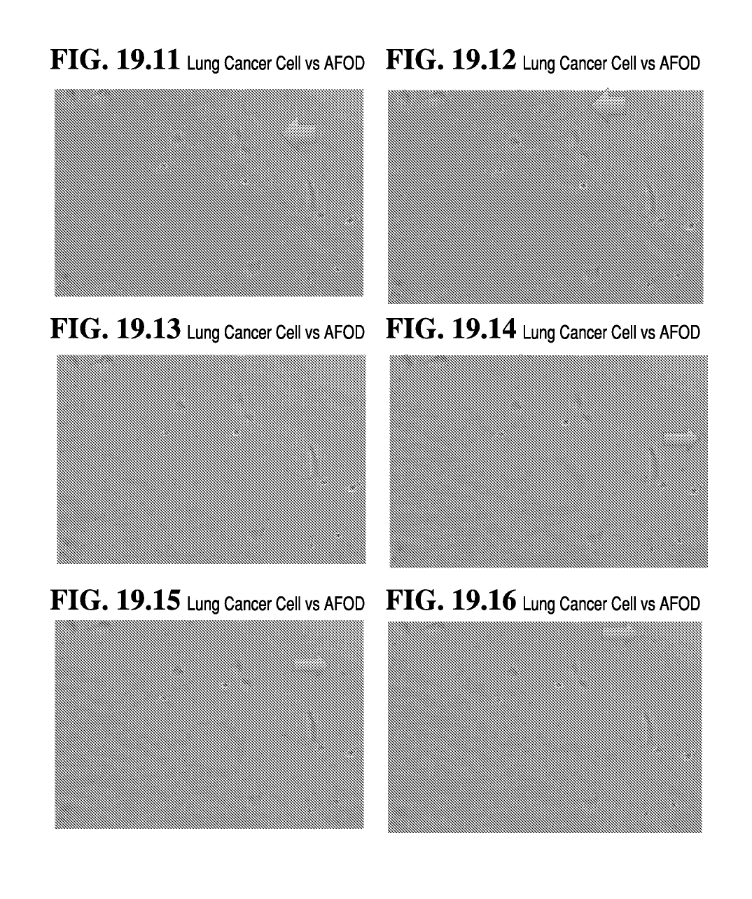

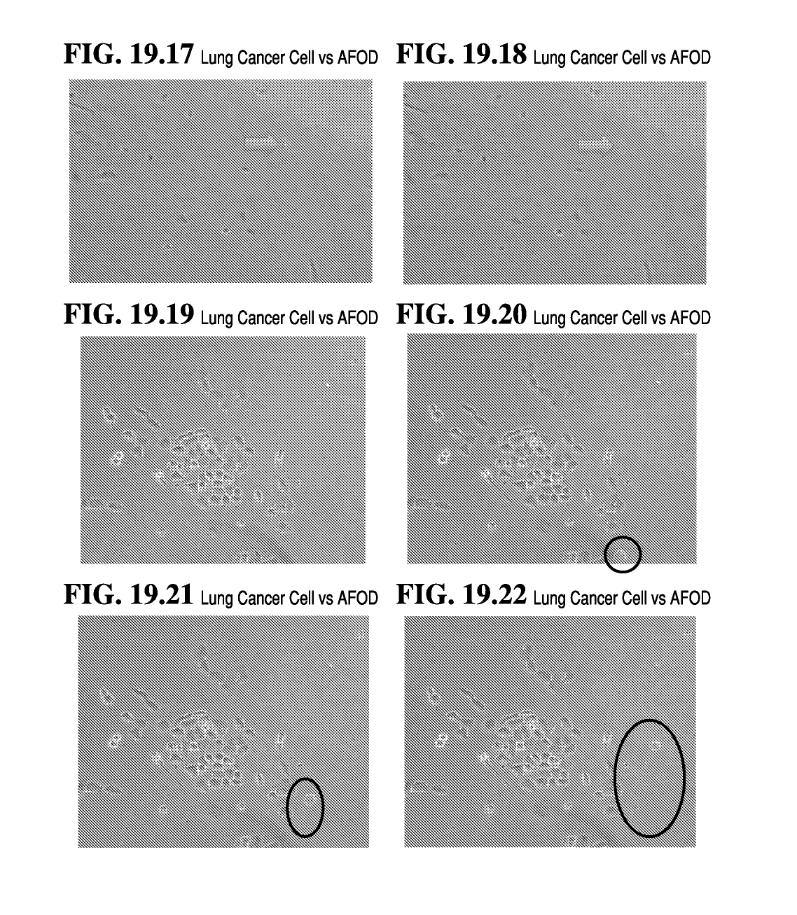

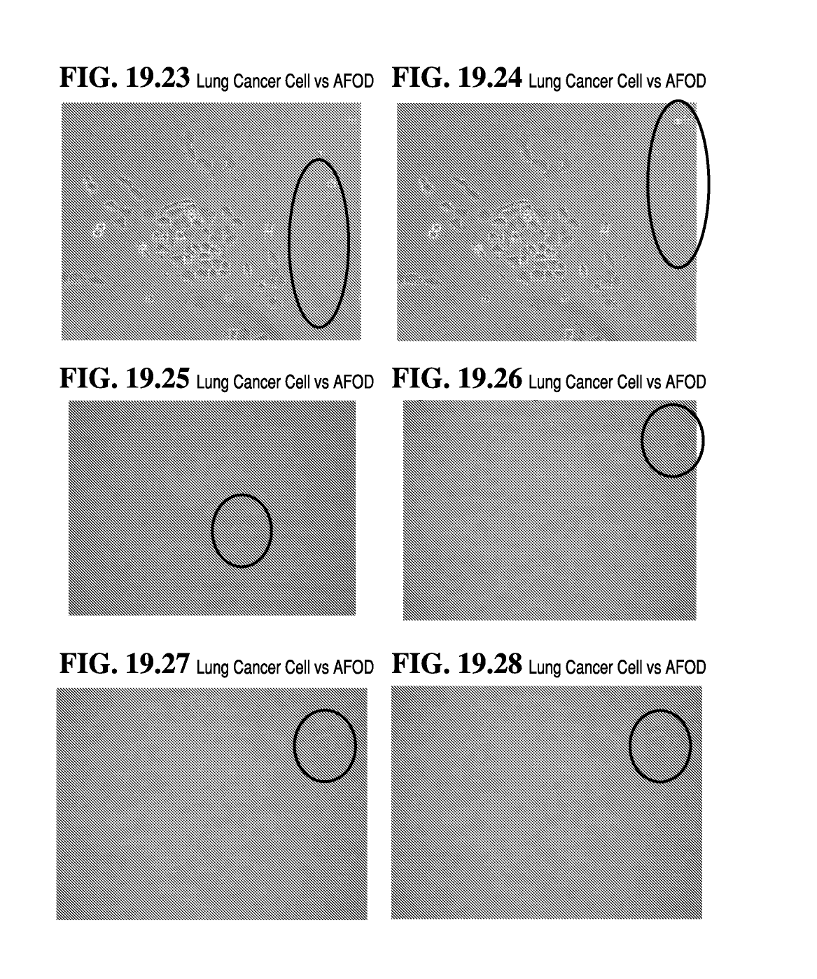

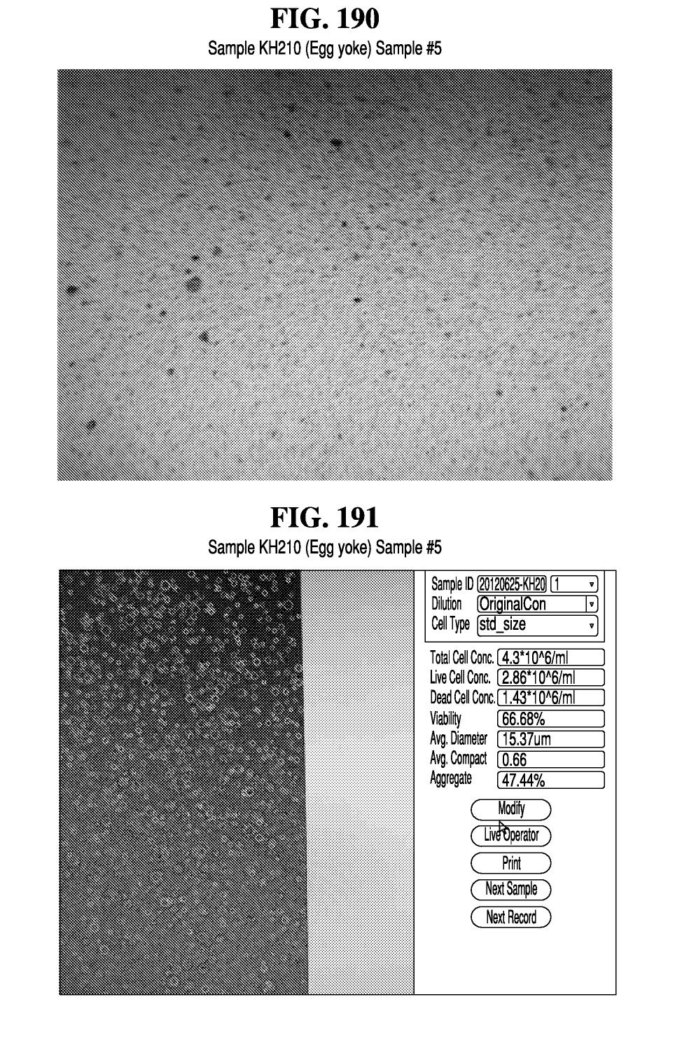

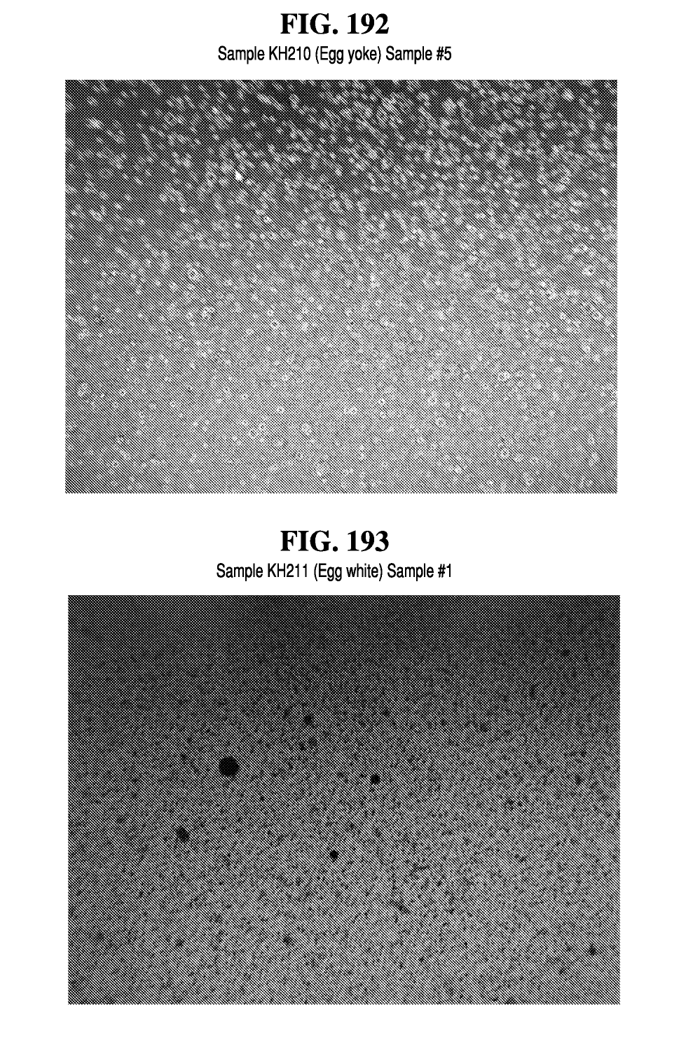

FIGS. 190, 191 and 19—KH 211 medium contains 22.8 g of paste of Egg white with 450 mL of WFI. Sample number 1, Tryptophan is added as a stabilizer.

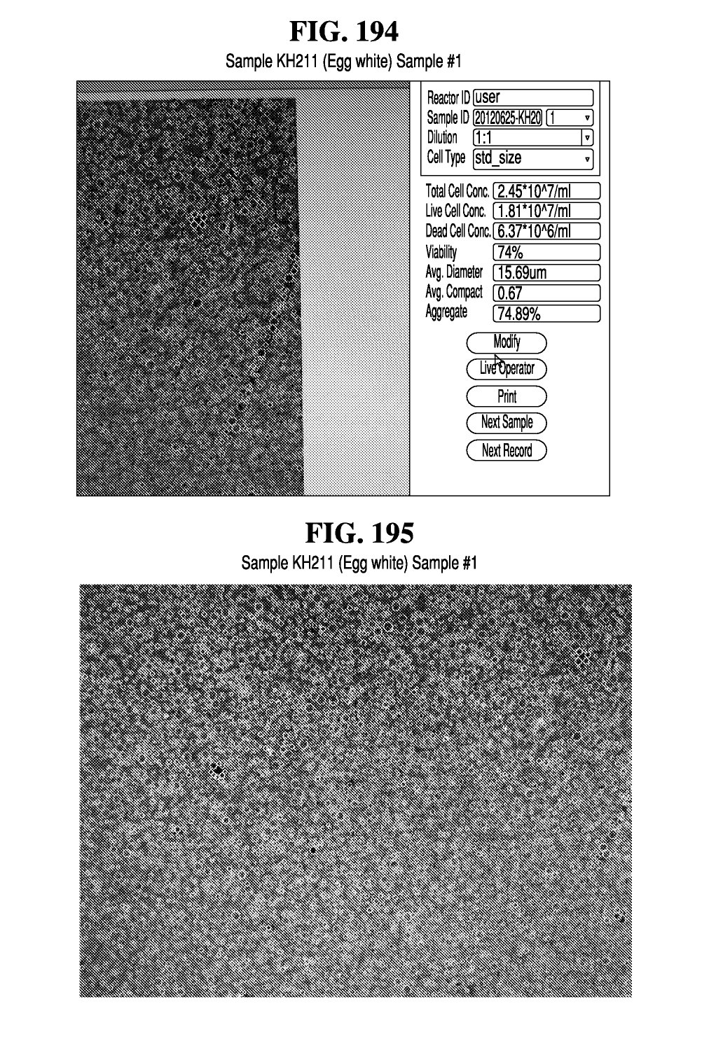

FIGS. 193, 194 and 19—KH211 medium sample number 2. Tryptophan is added as a stabilizer.

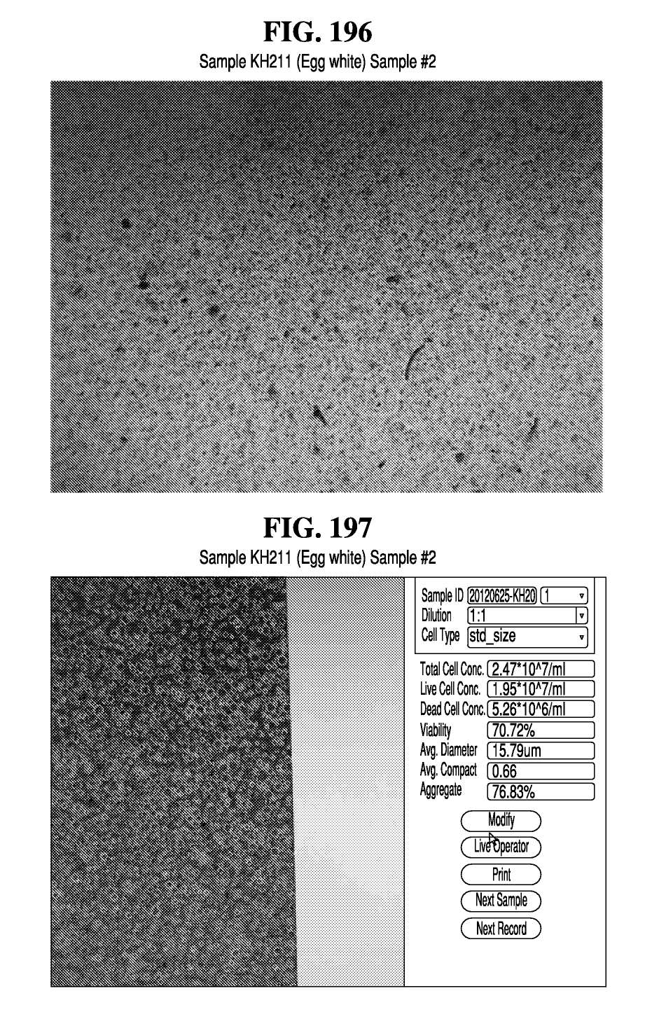

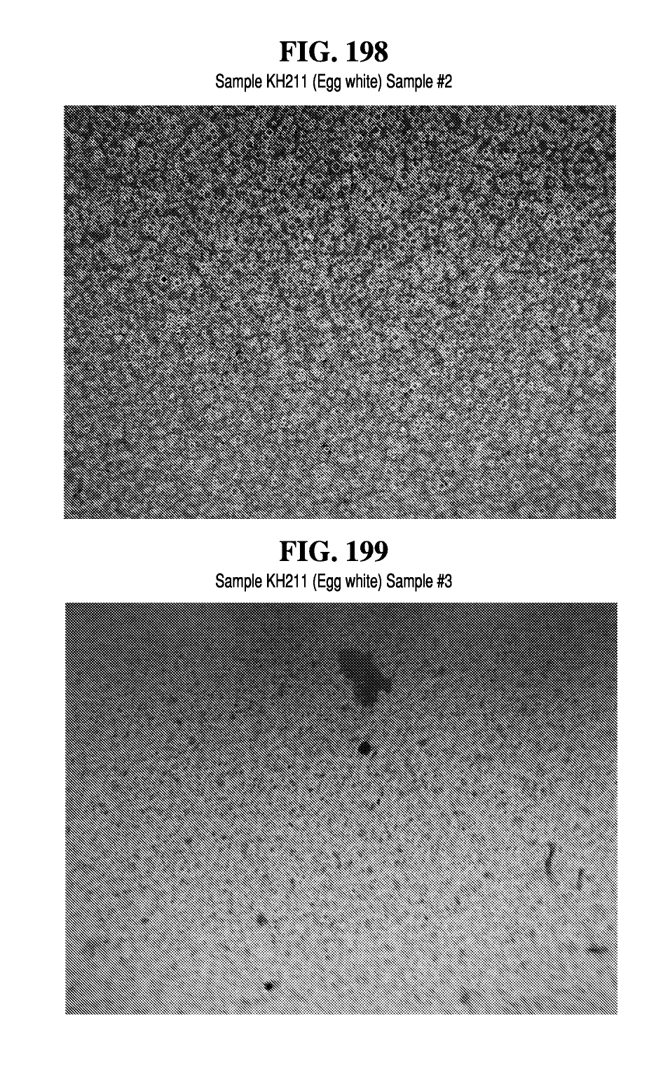

FIGS. 196, 197 and 19—KH211 medium sample number 3. Tryptophan is added as a stabilizer.

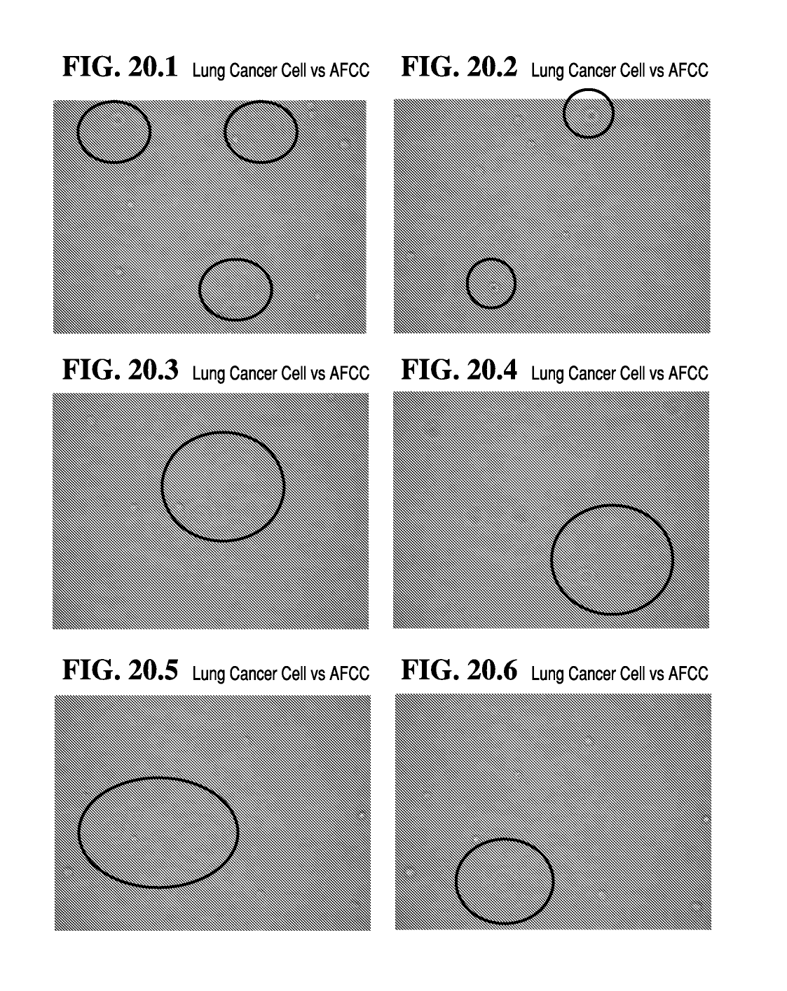

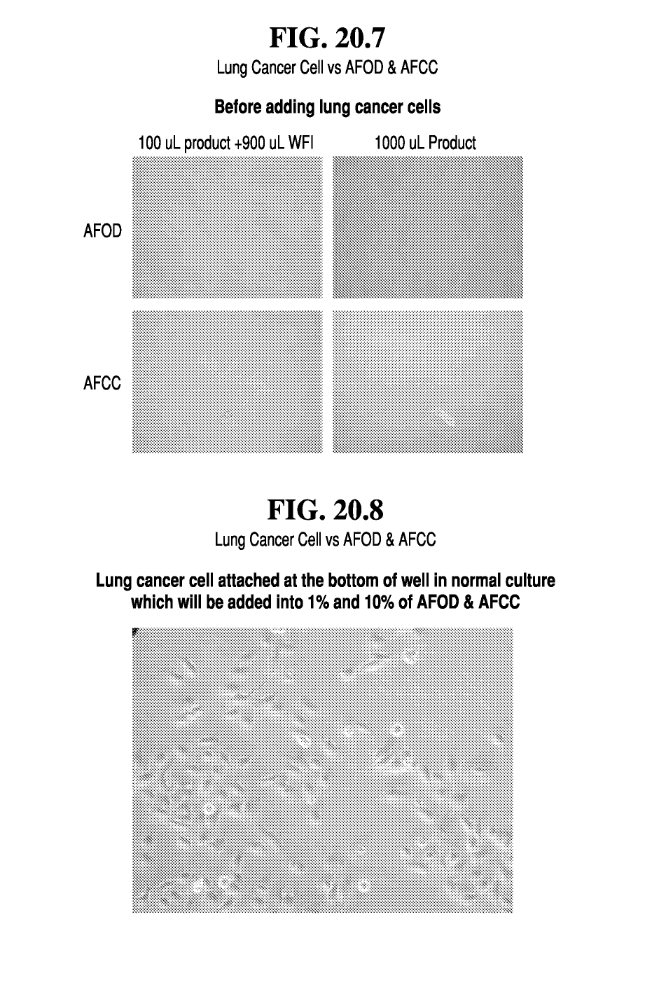

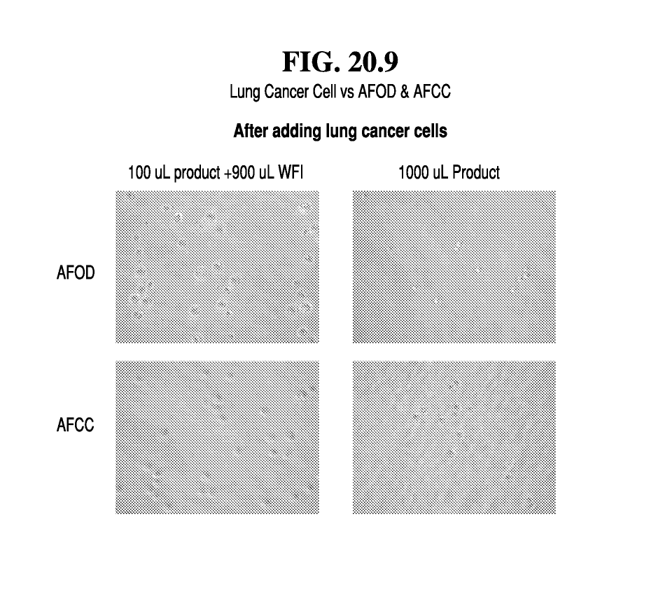

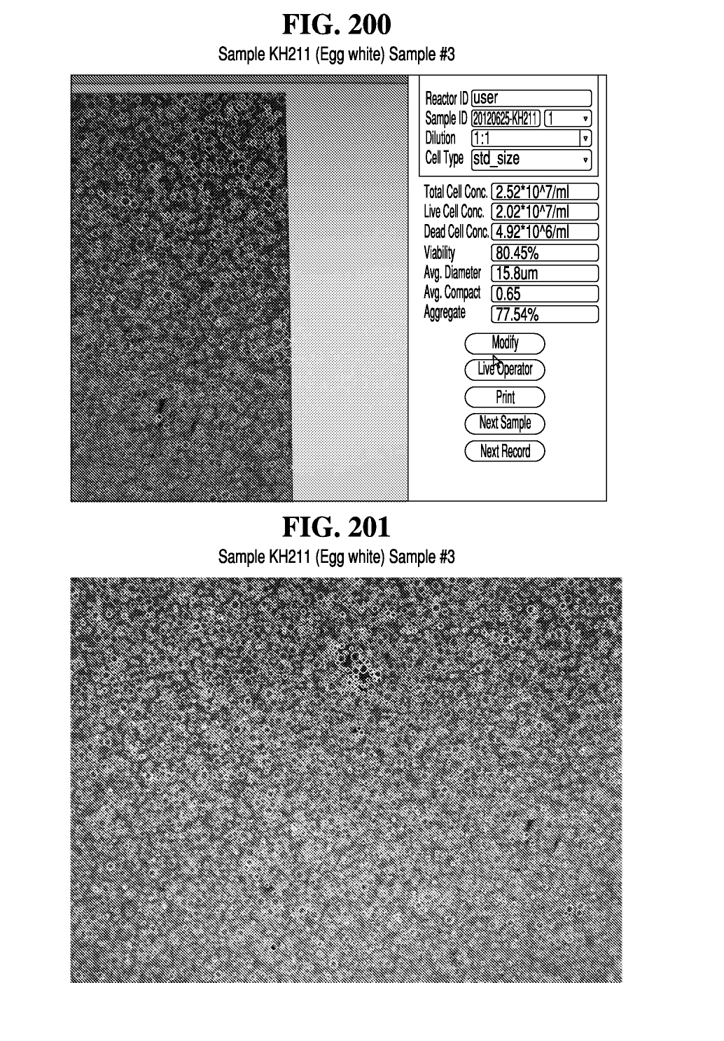

FIGS. 199, 200 and 20—KH211 medium sample number 4. Without Tryptophan.

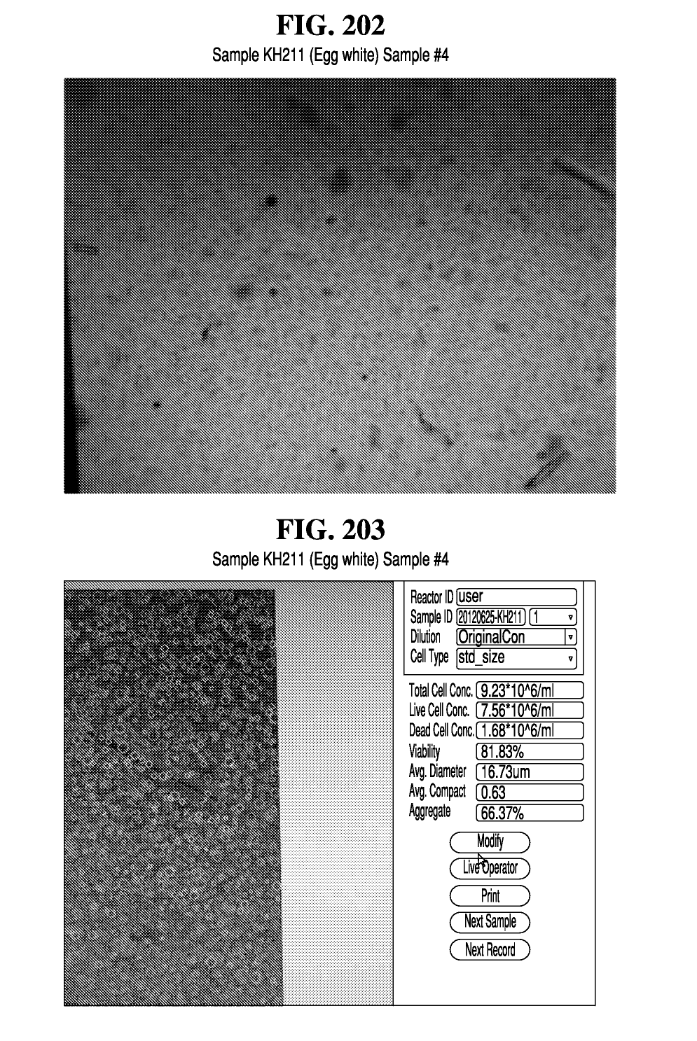

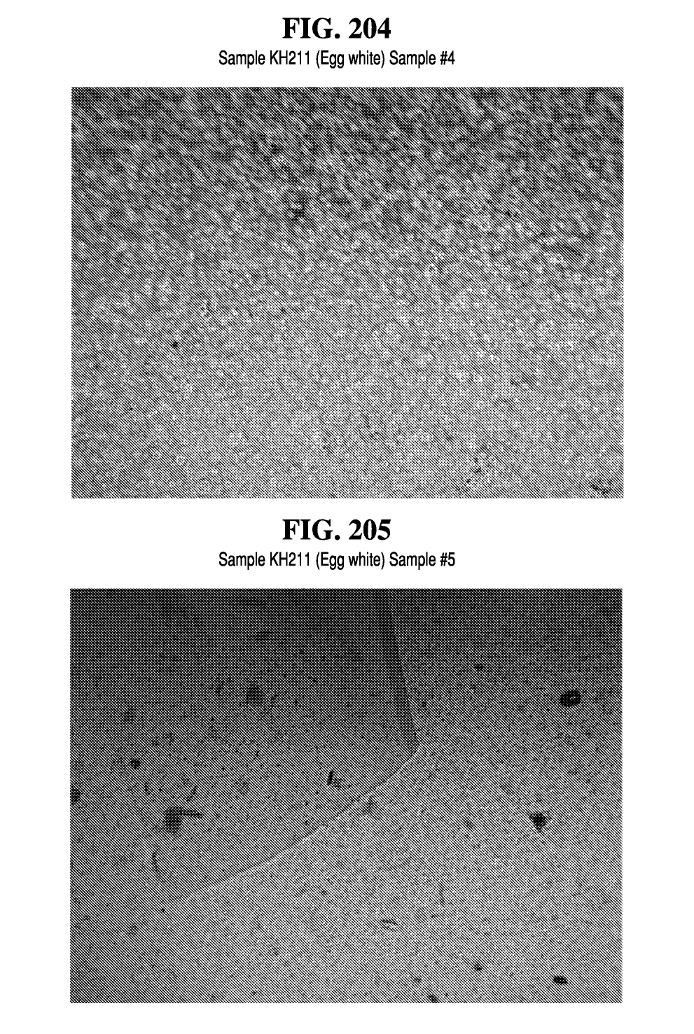

FIGS. 202, 203 and 20—KH211 medium sample number 5. Without Tryptophan.

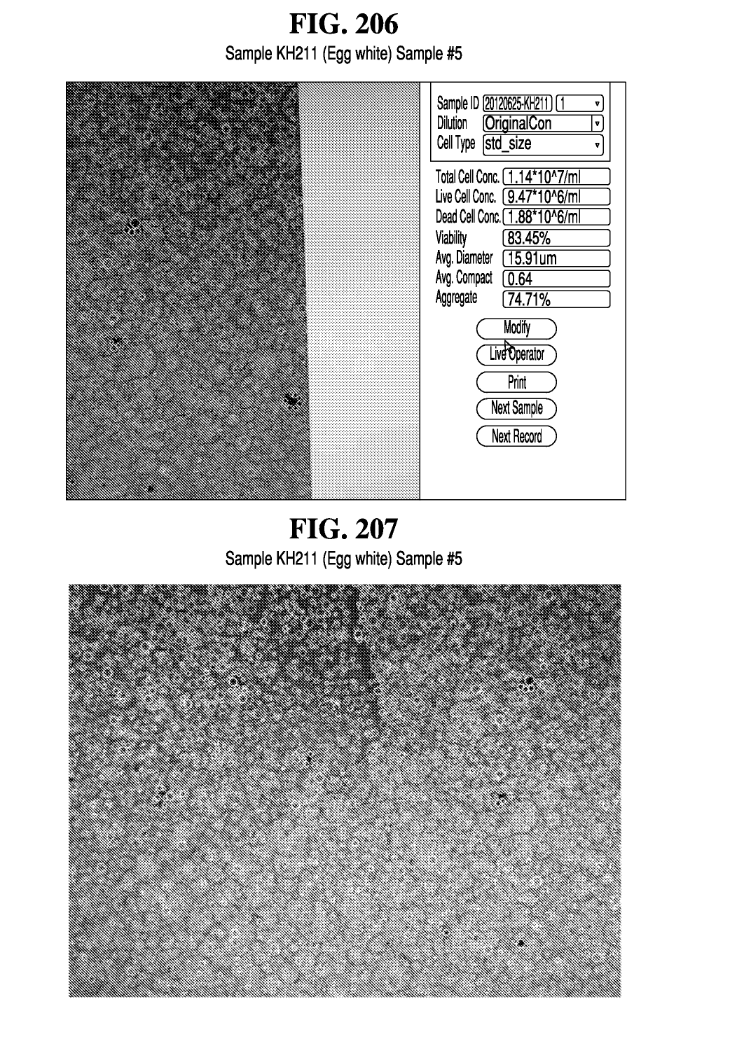

FIGS. 205, 206 and 20—KH 212 medium contains 27.1 g of paste of Shanghai Crab with 540 mL of WFI. Sample number 1, Tryptophan is added as a stabilizer.

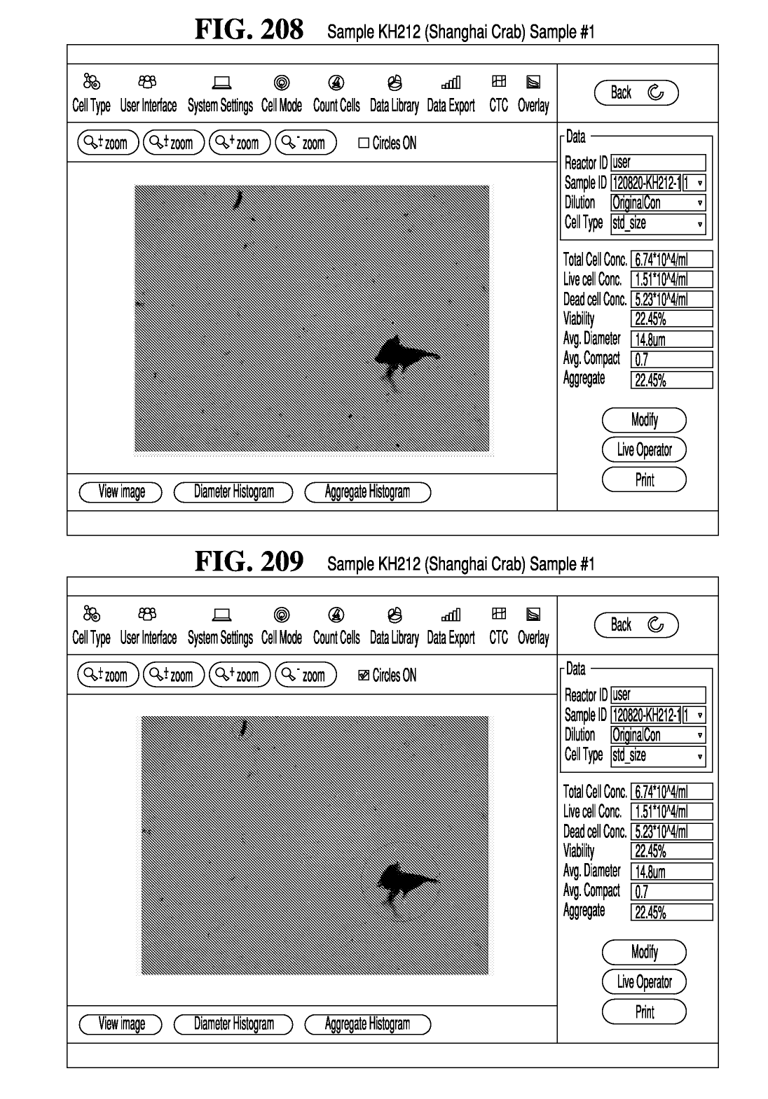

FIGS. 208 and 20—KH 213 medium contains 17.1 g of paste of Crawfish with 340 mL of WFI. Sample number 1, Tryptophan is added as a stabilizer.

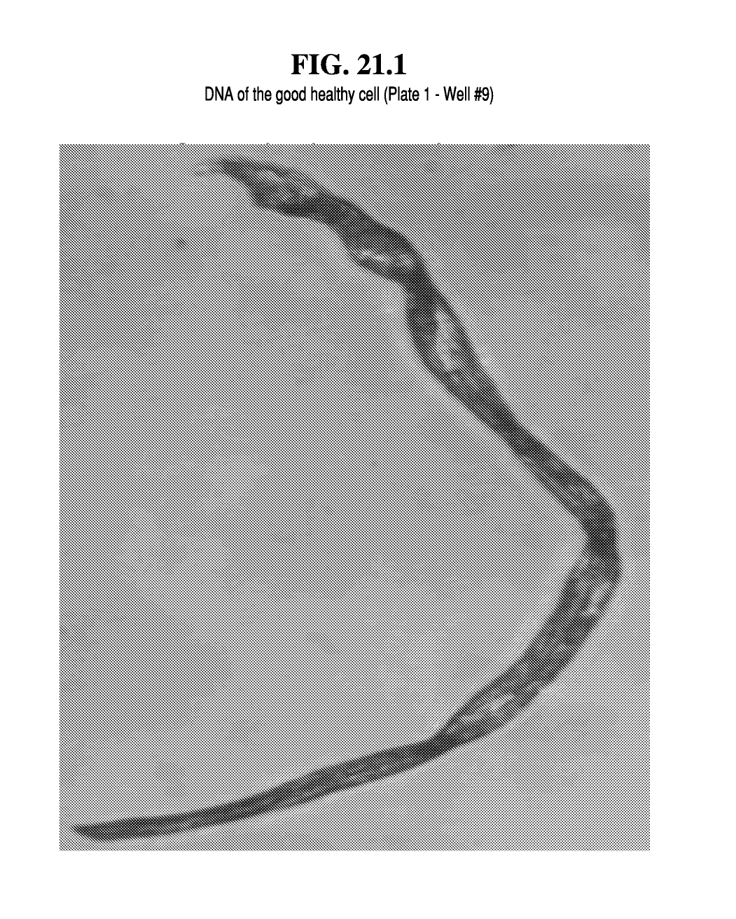

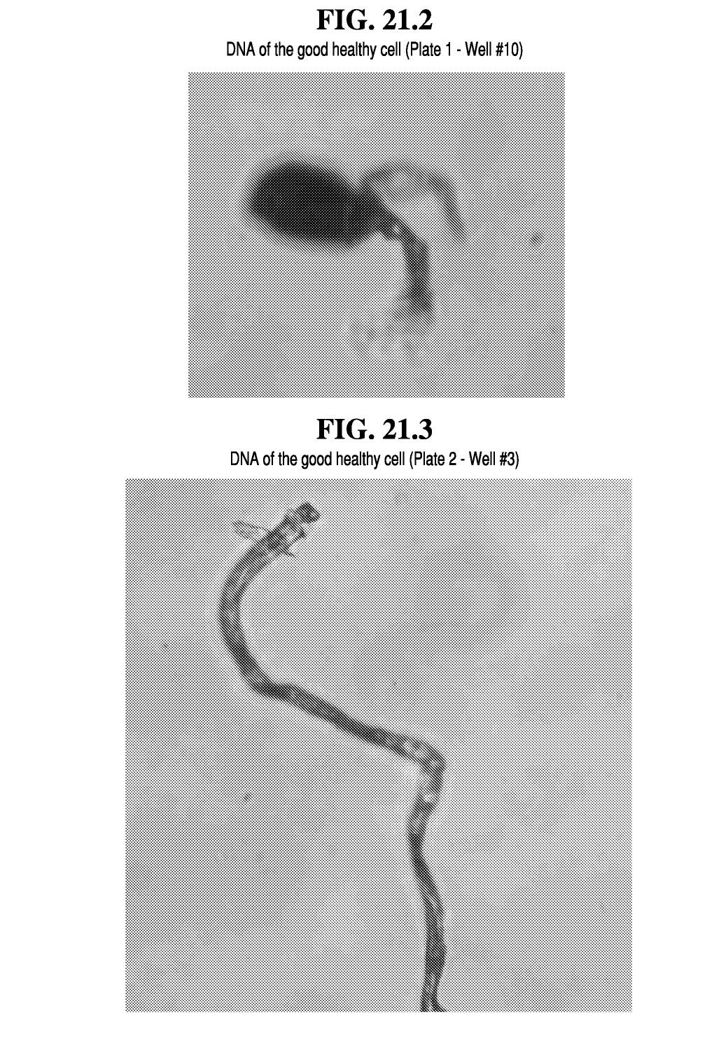

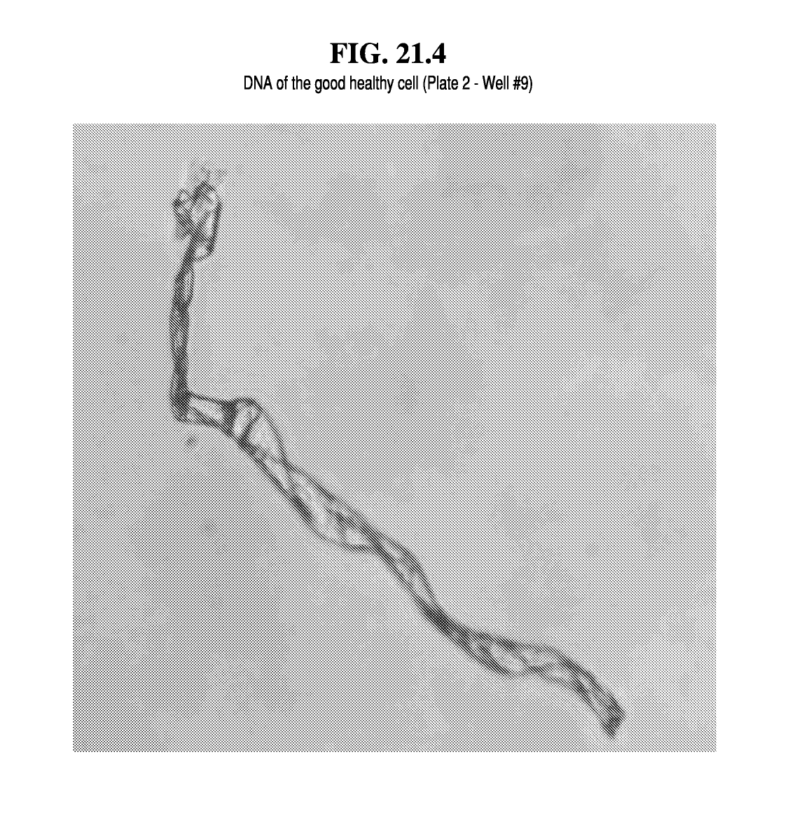

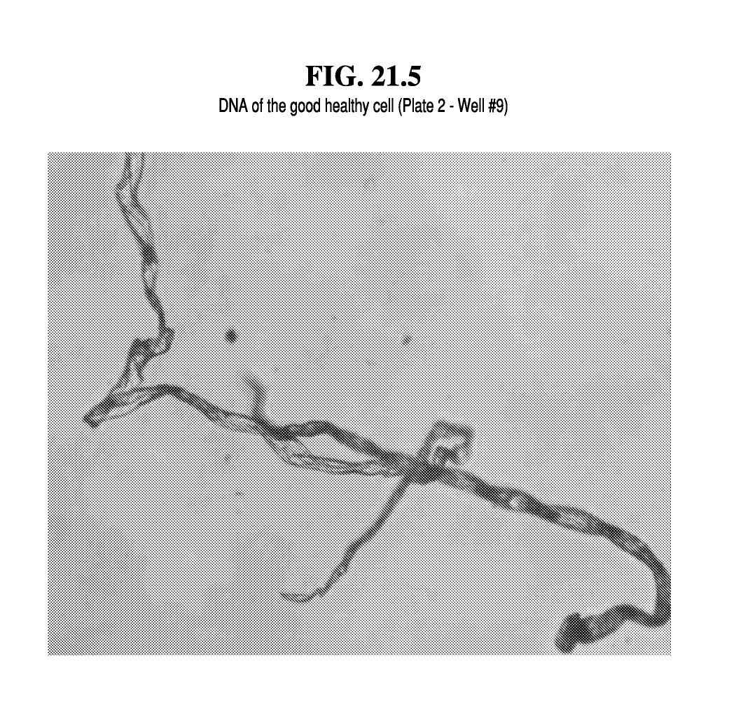

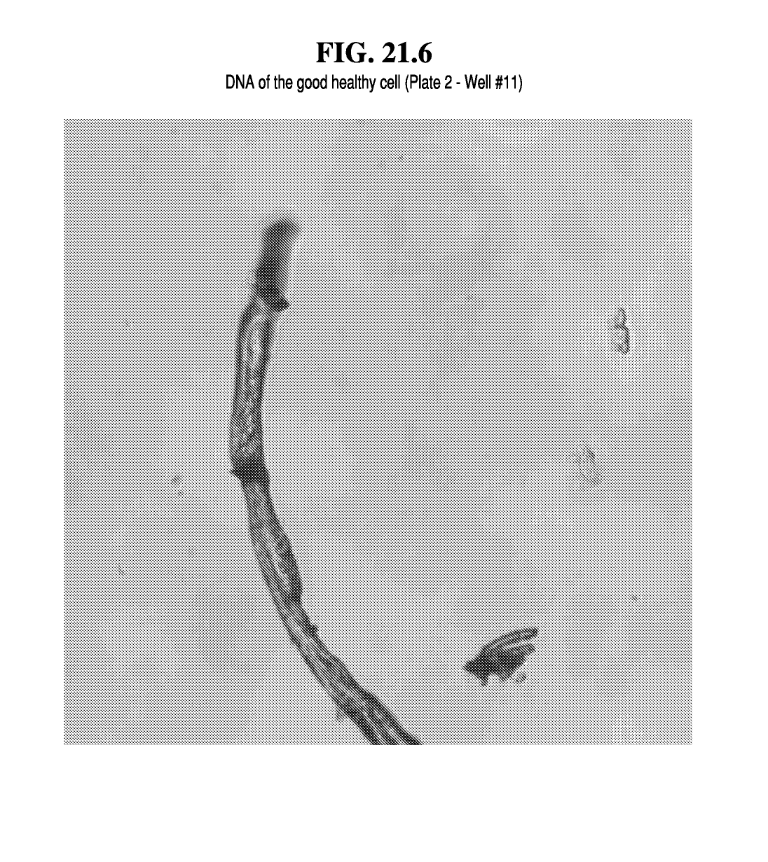

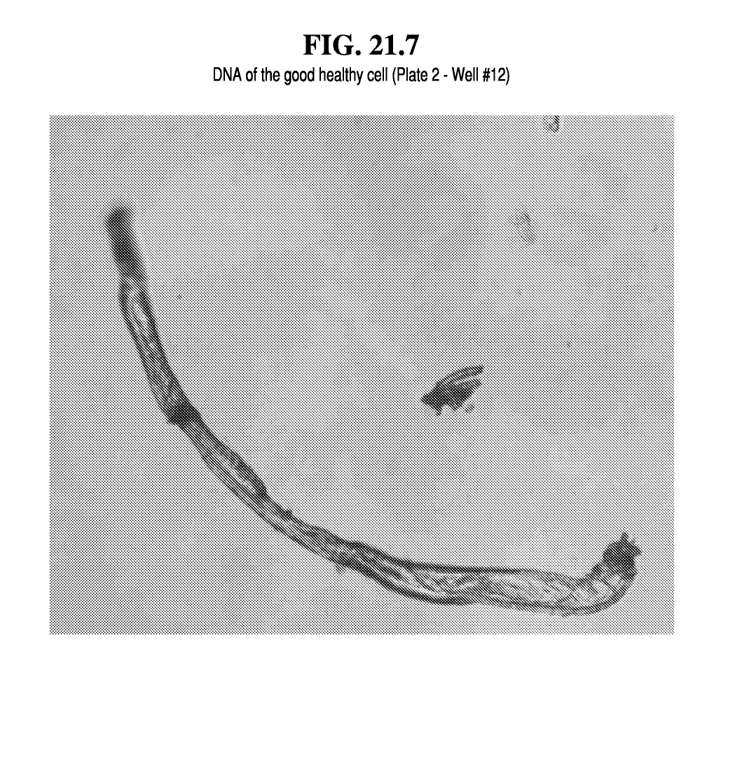

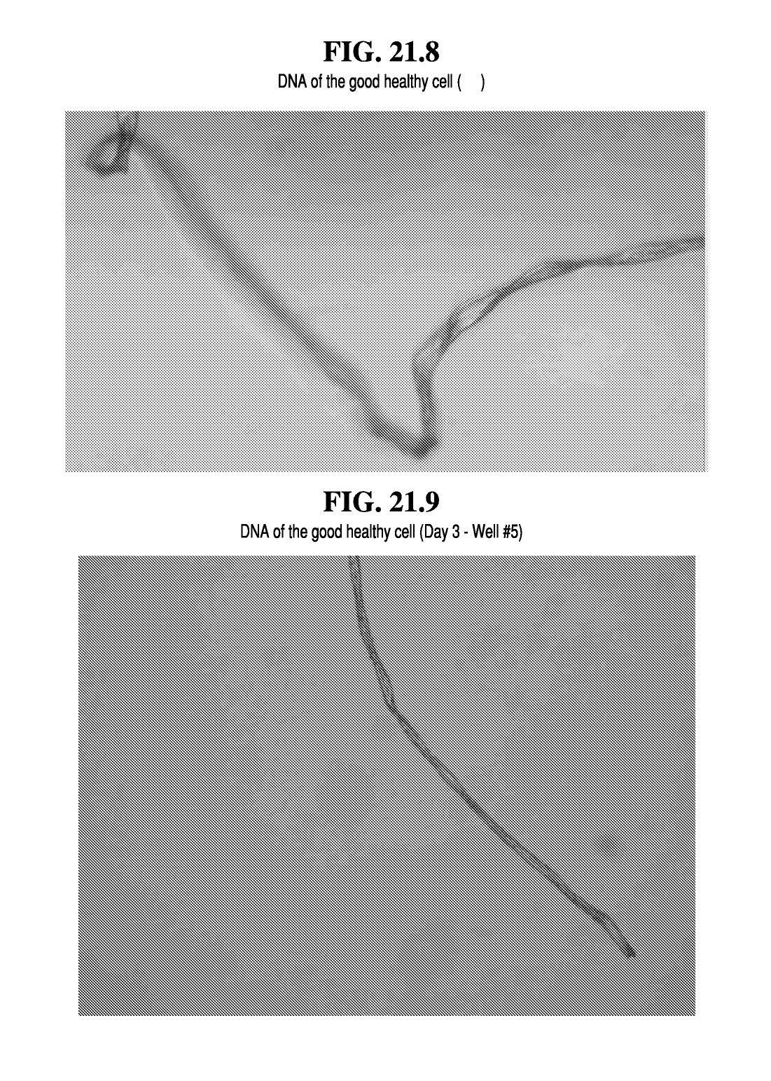

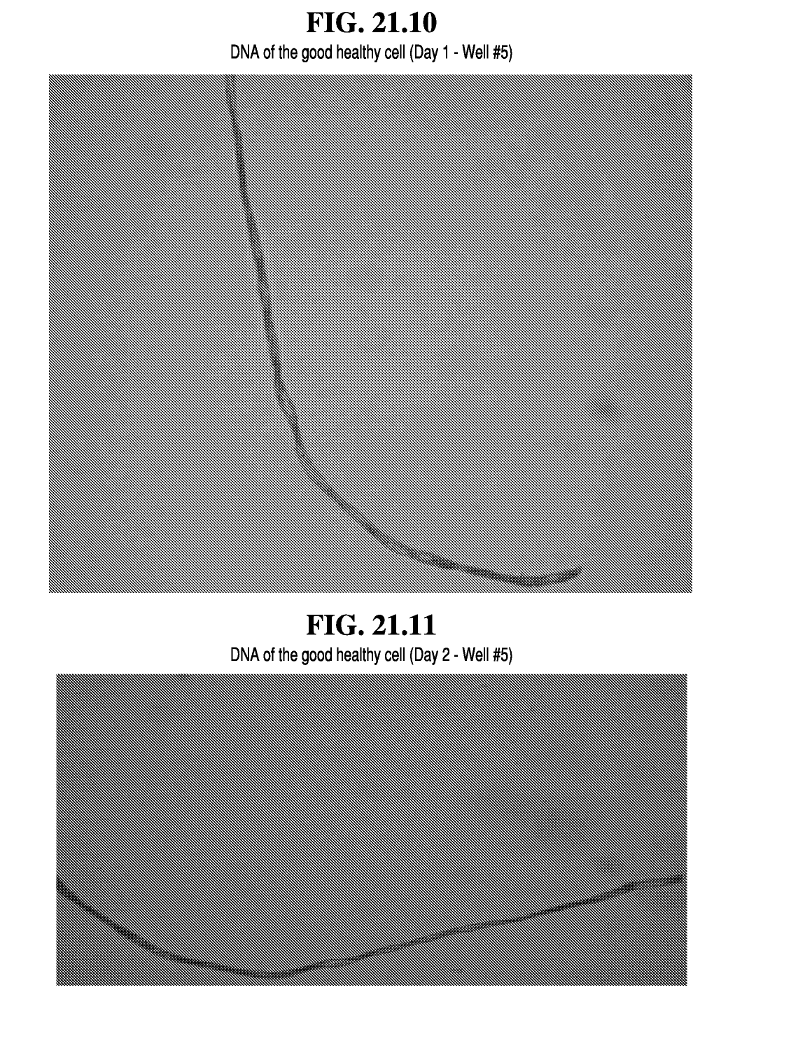

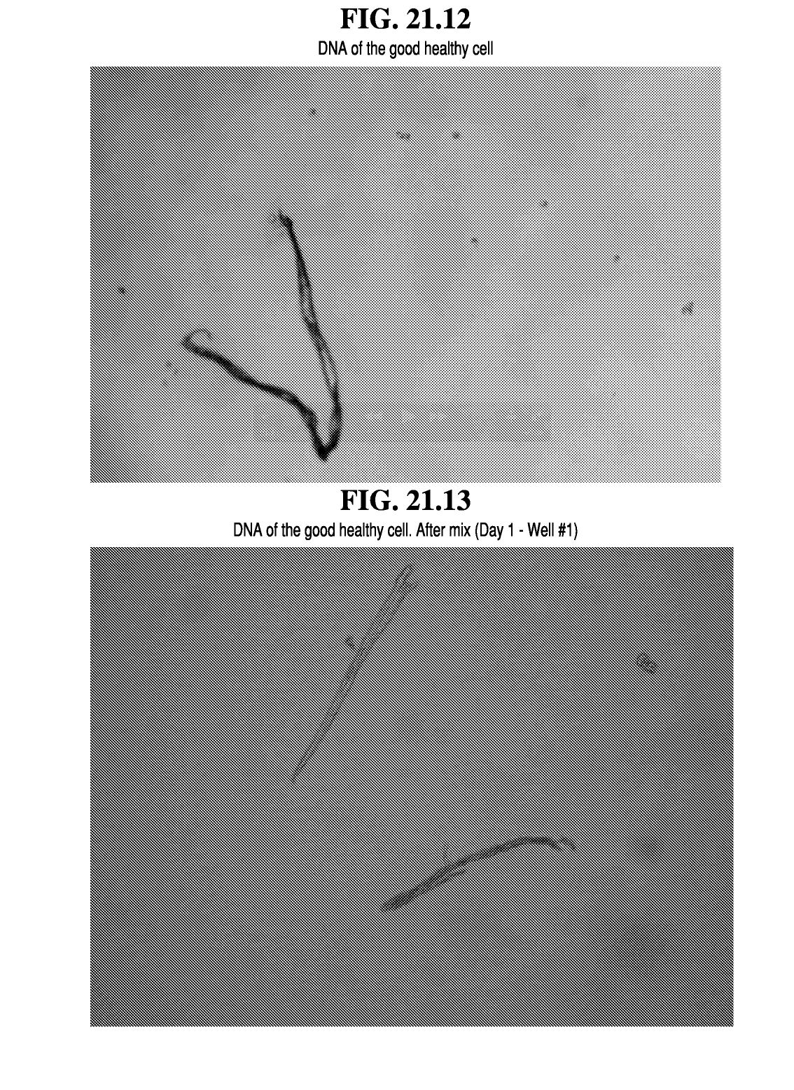

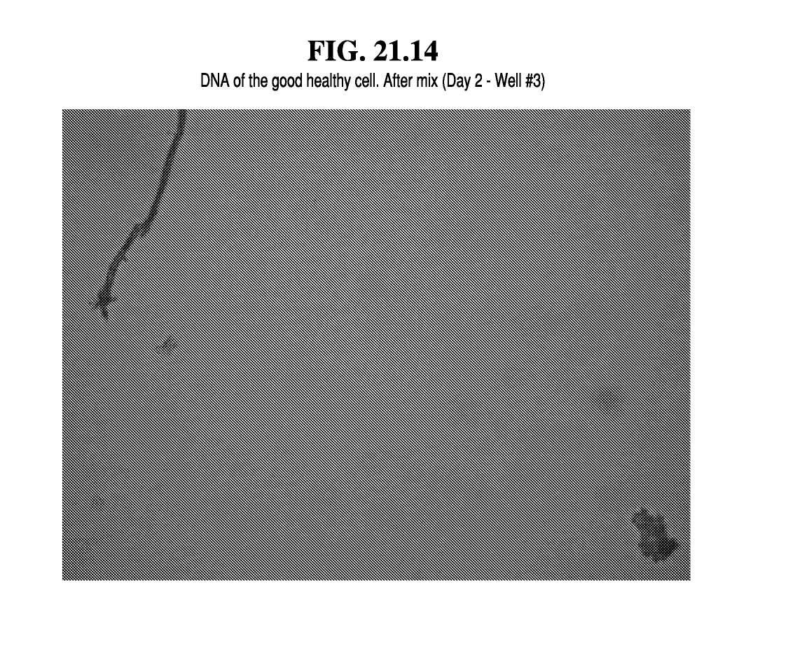

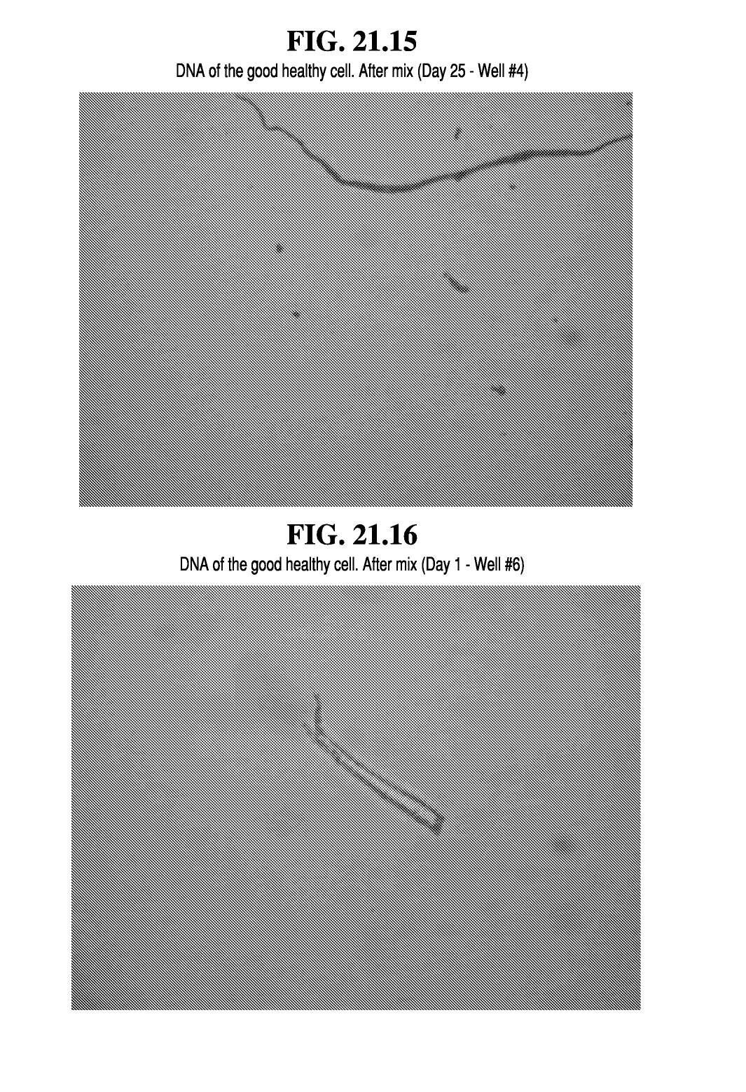

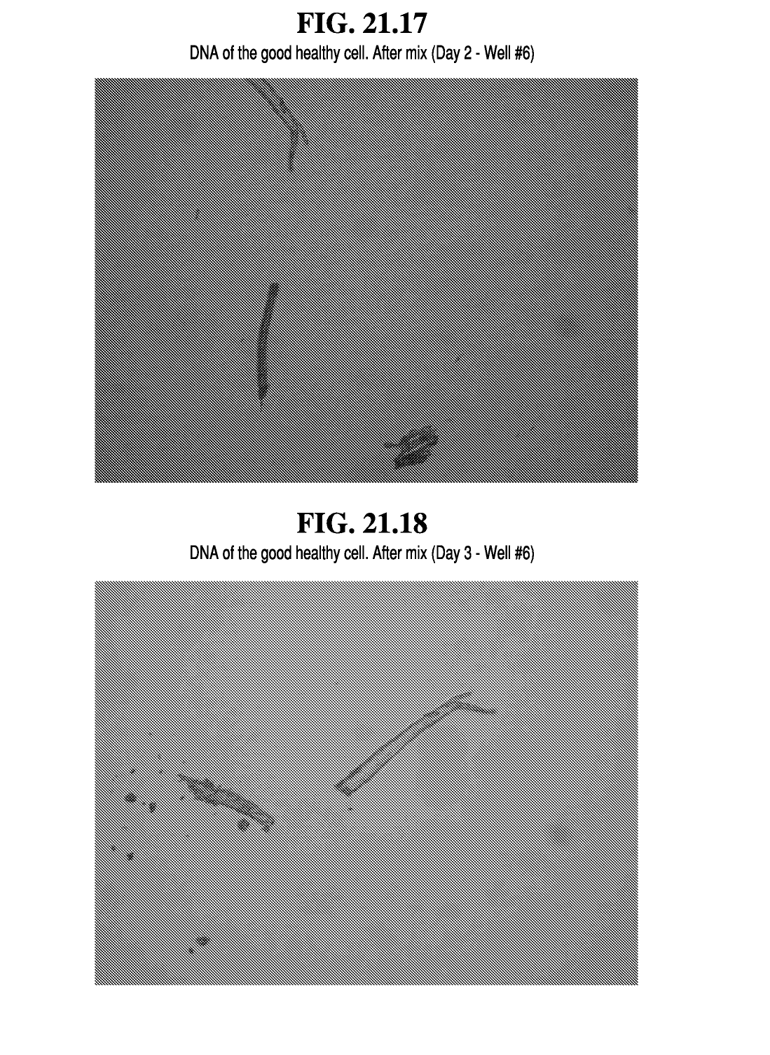

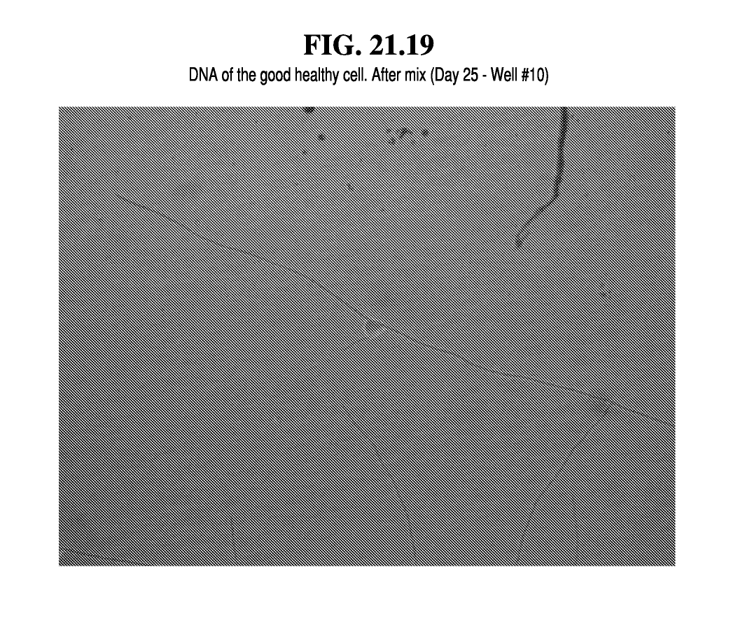

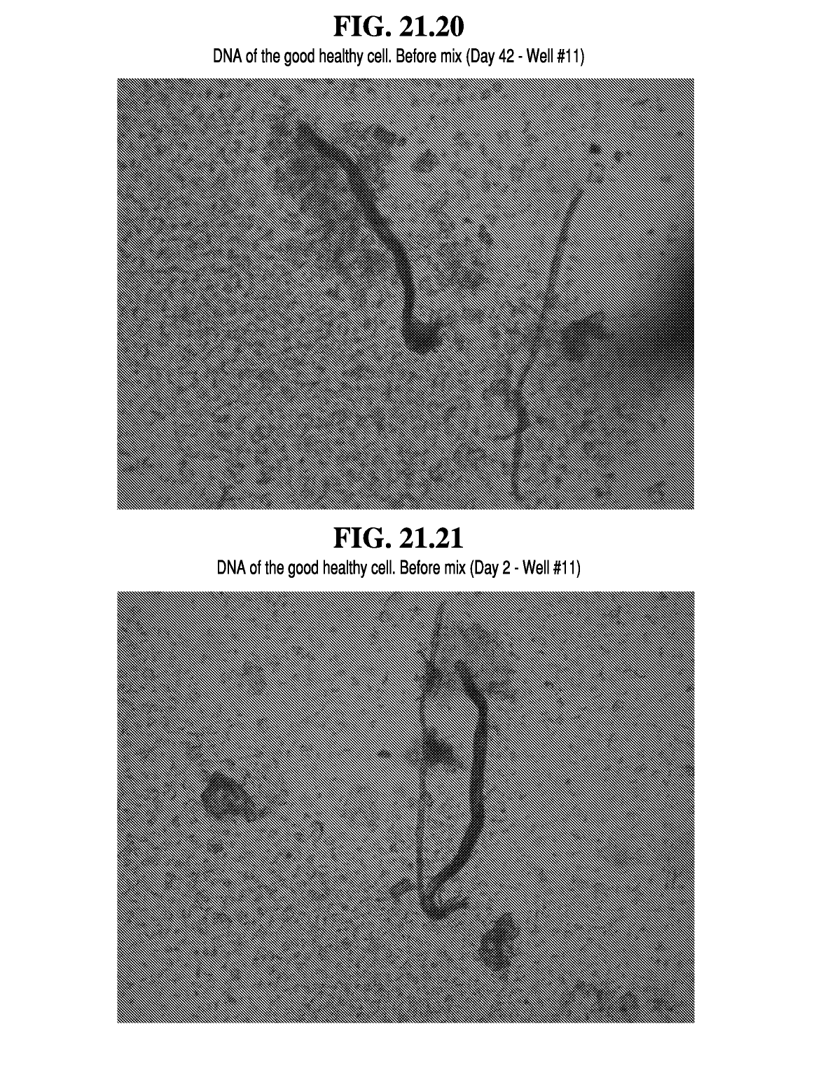

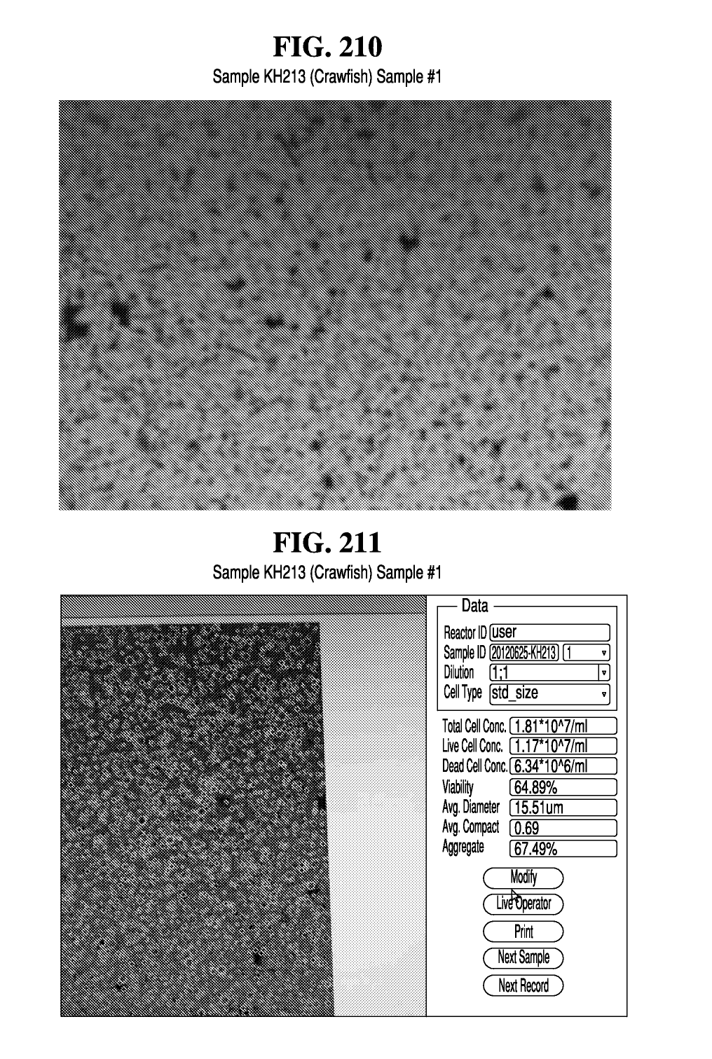

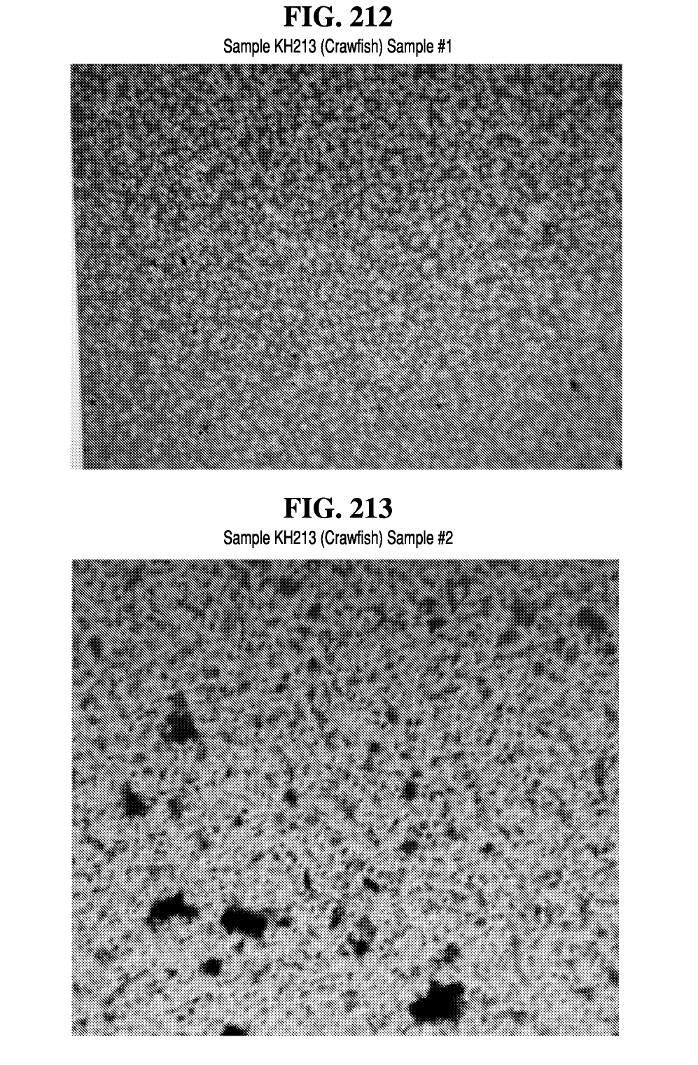

FIGS. 210, 211 and 21—KH213 medium sample number 2. Tryptophan is added as a stabilizer.

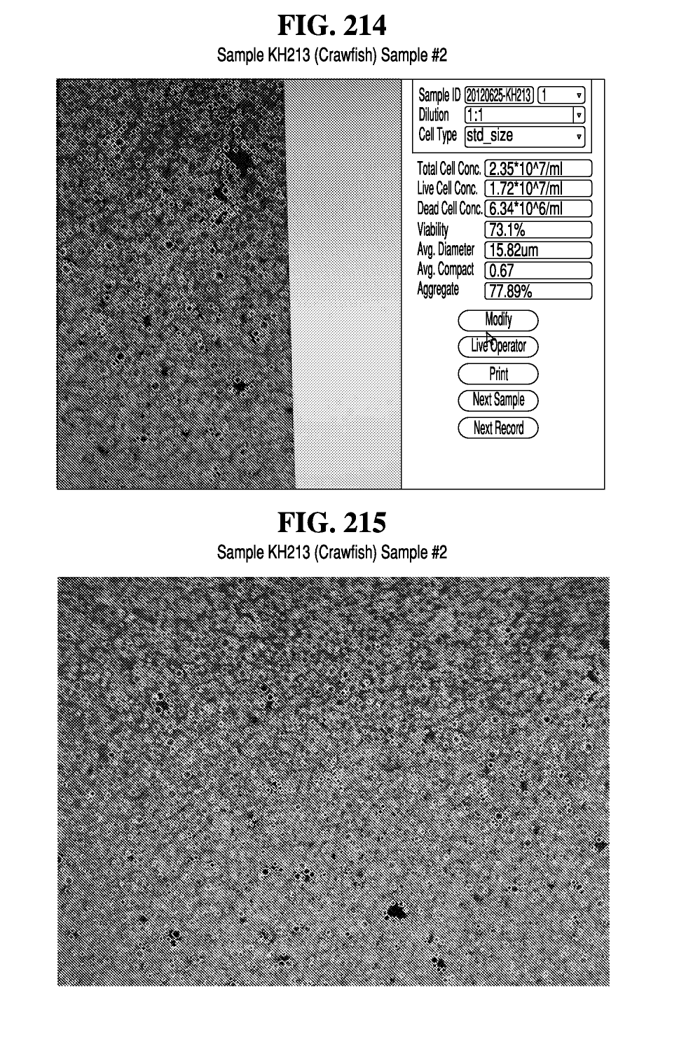

FIGS. 213, 214 and 21—KH213 medium sample number 3. Tryptophan is added as a stabilizer.

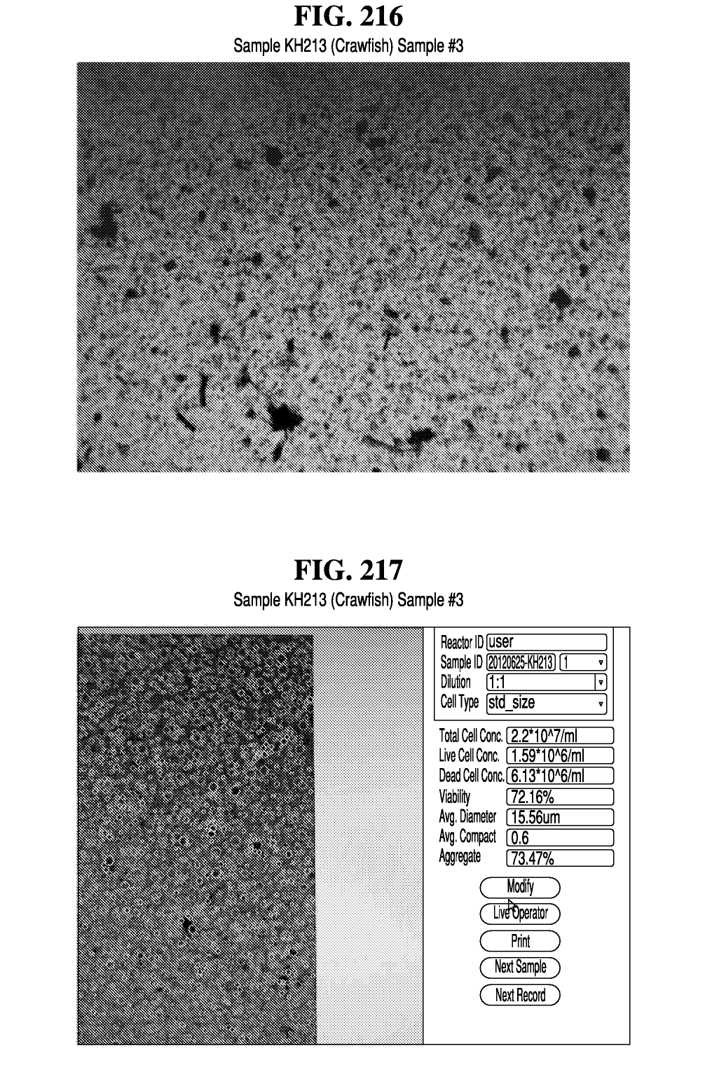

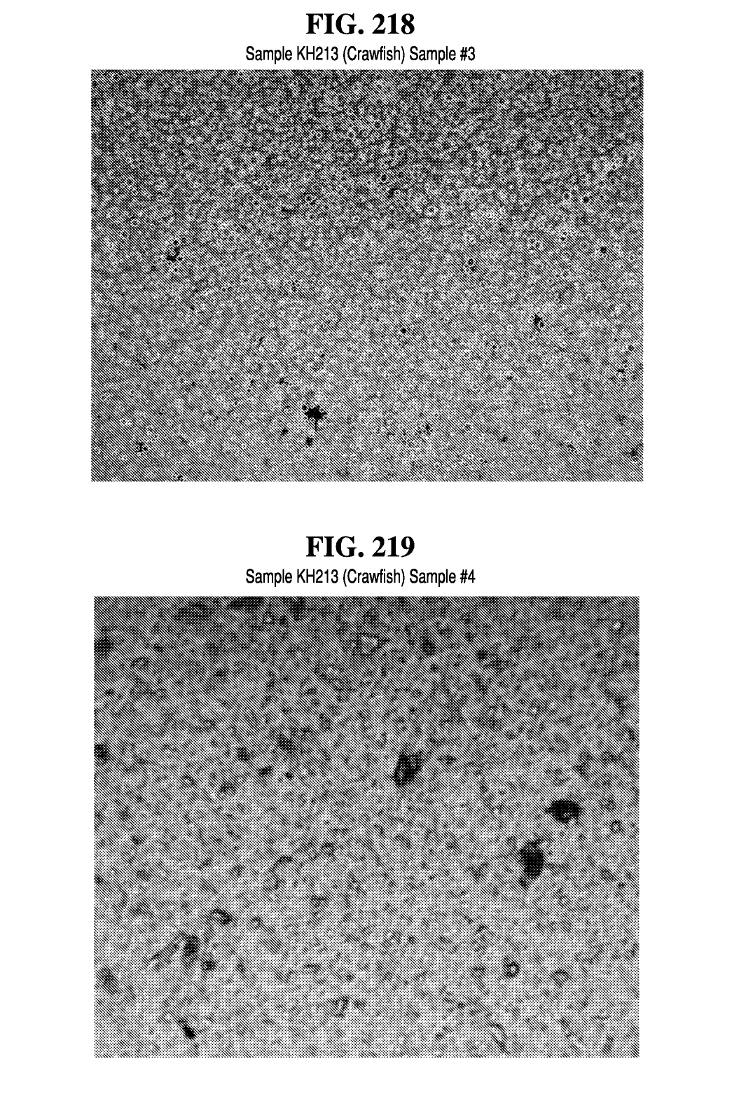

FIGS. 216, 217 and 21—KH213 medium sample number 4. Without Tryptophan.

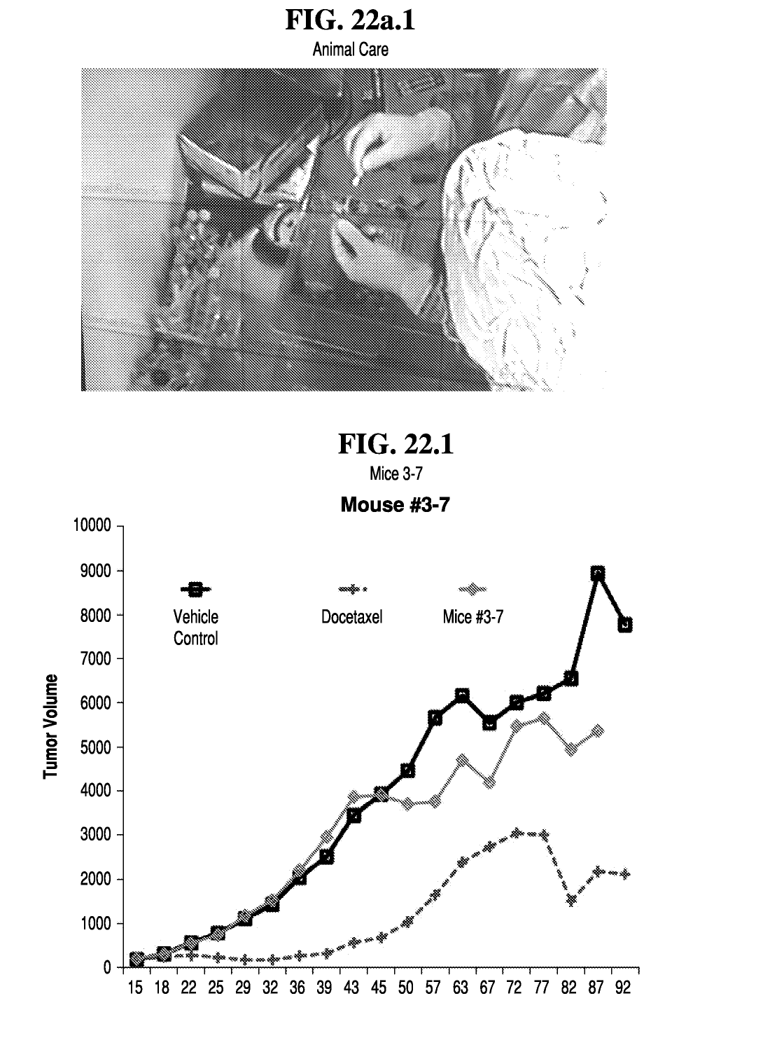

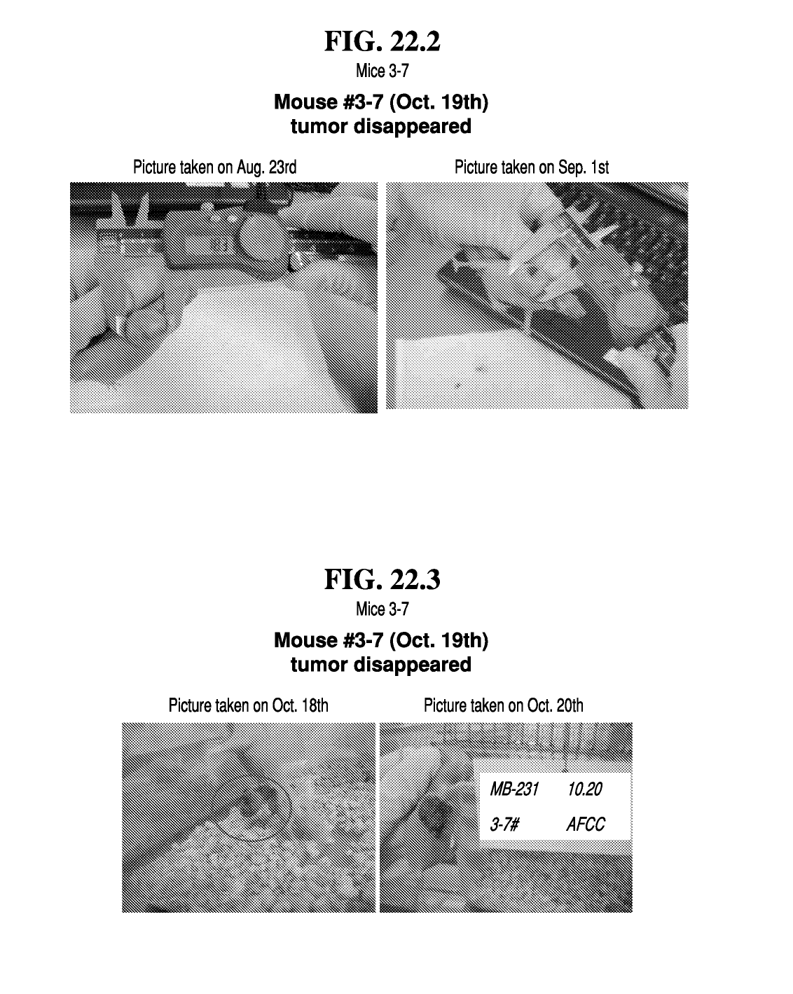

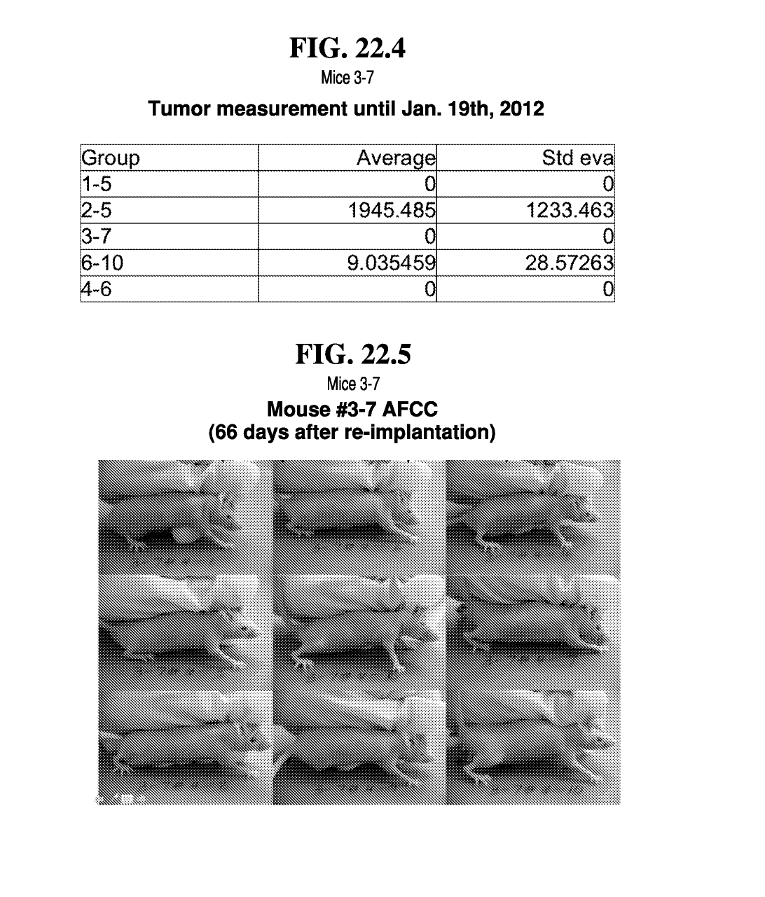

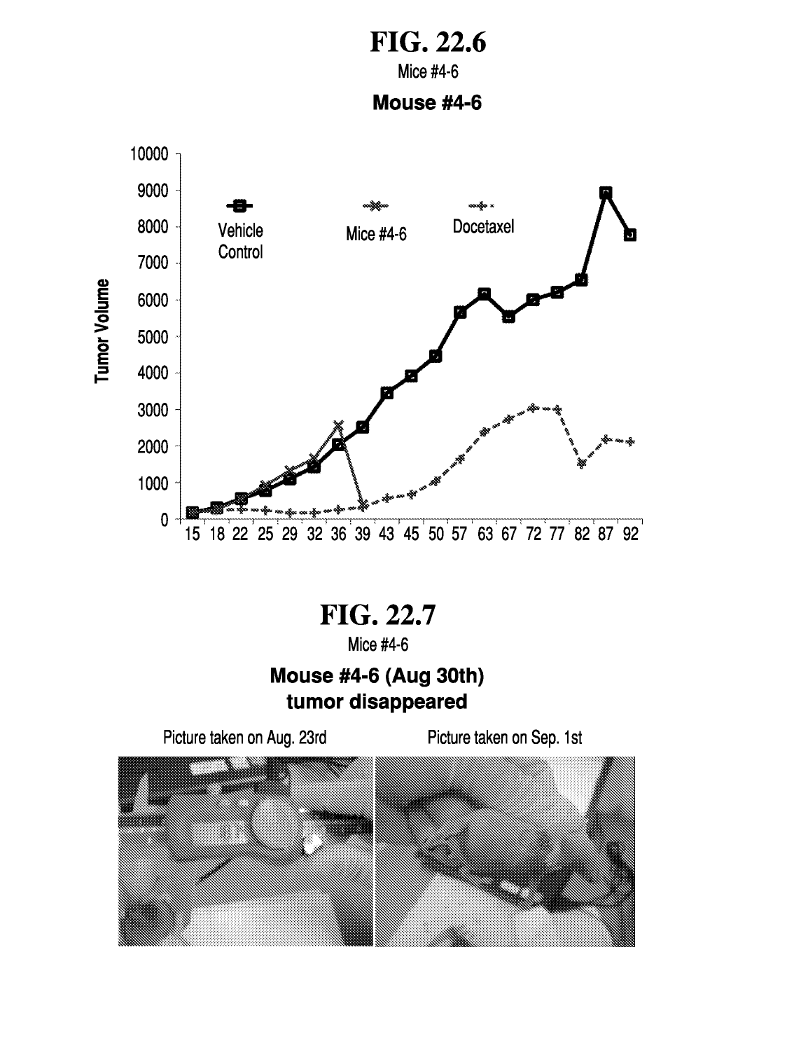

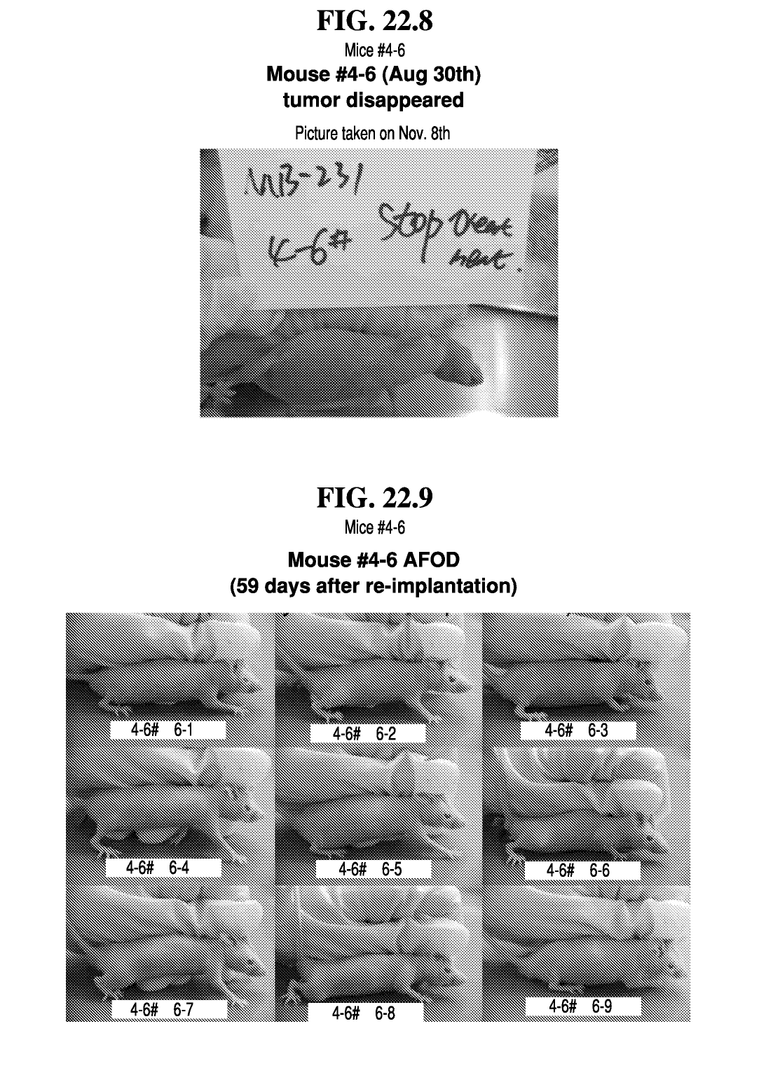

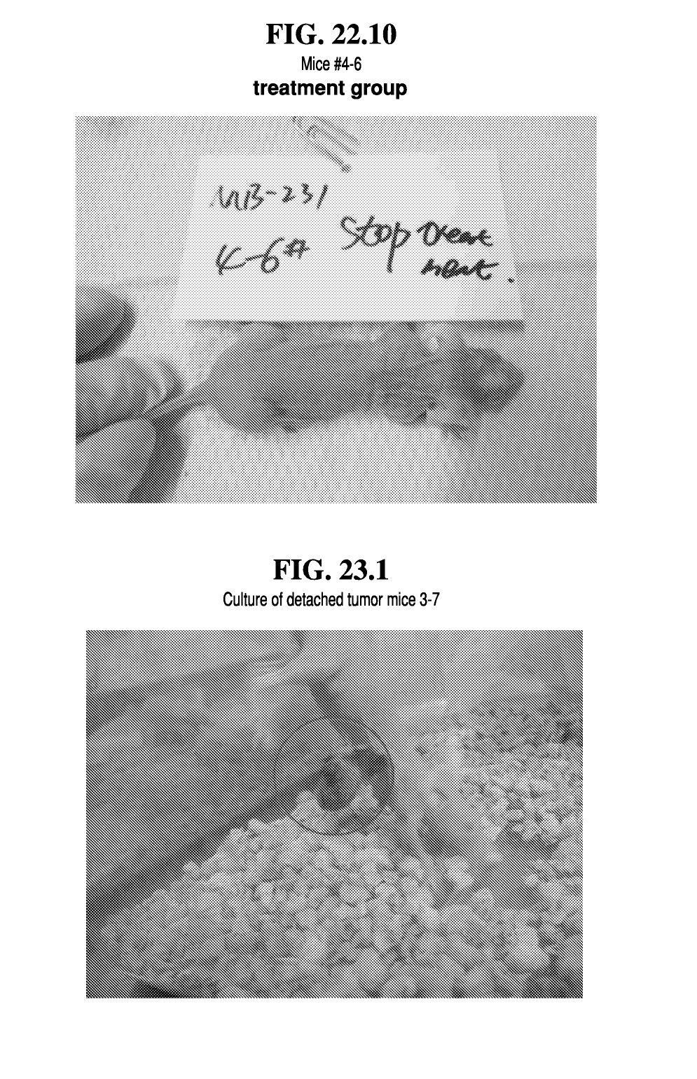

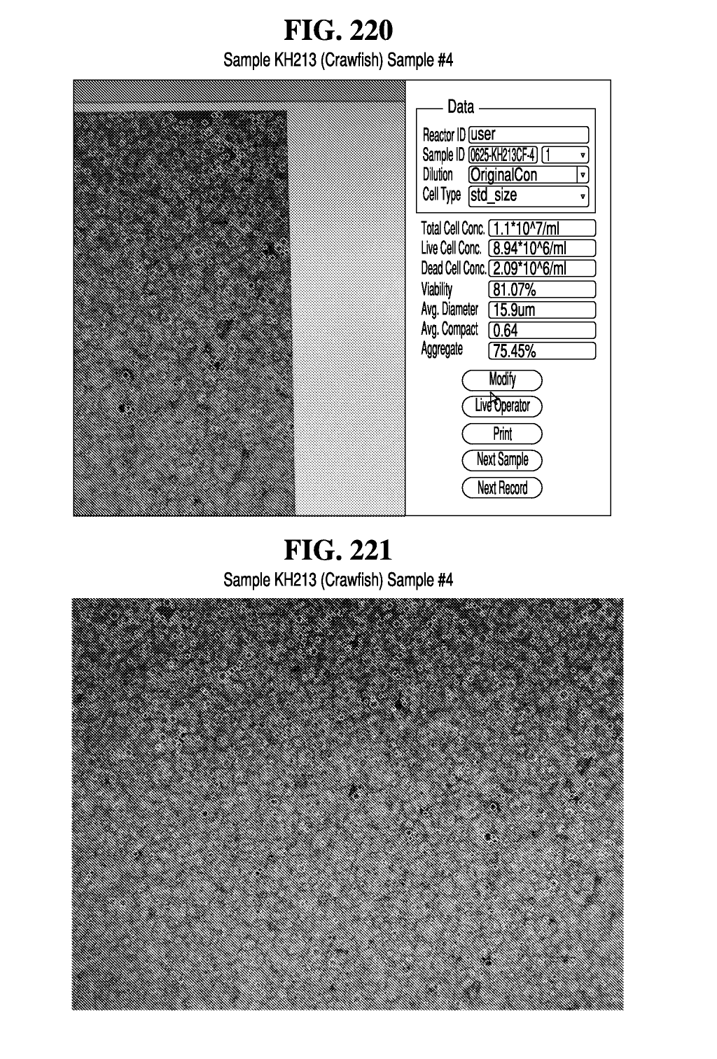

FIGS. 219, 220 and 22—KH213 medium sample number 5. Without Tryptophan.

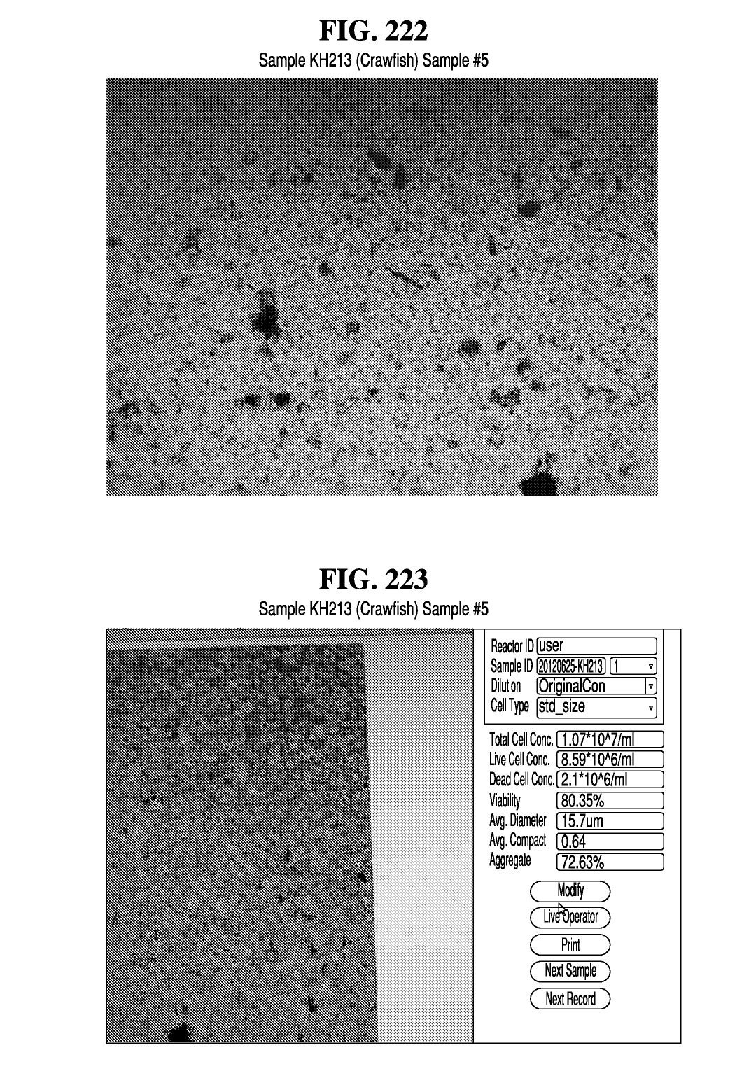

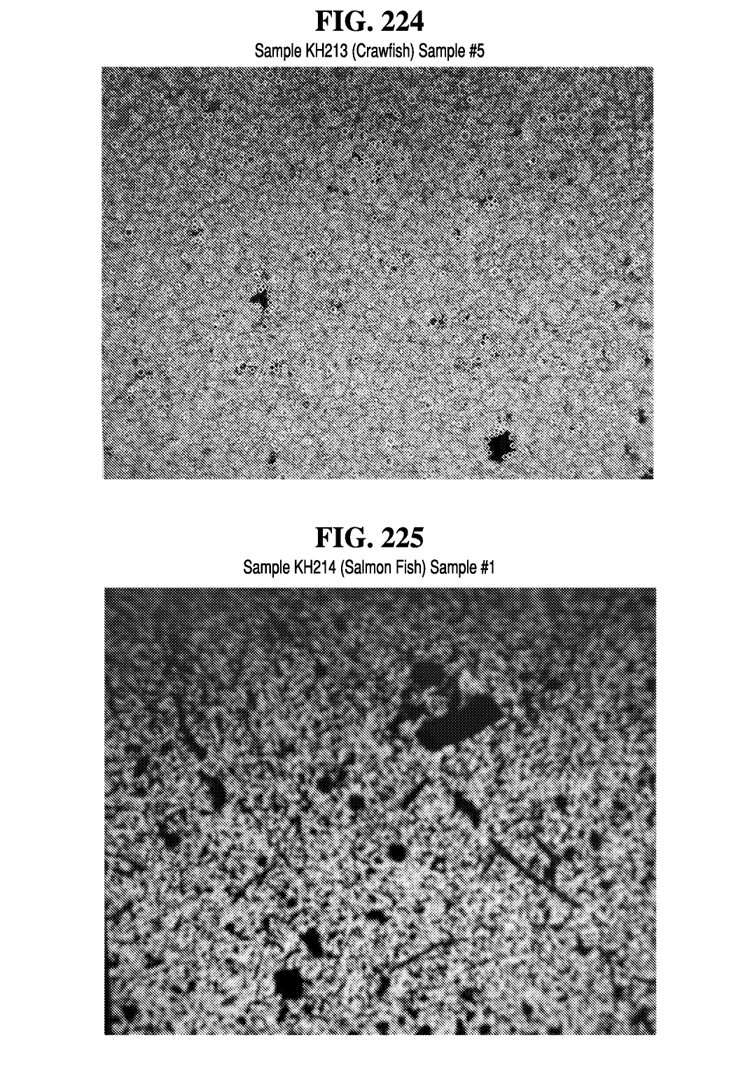

FIGS. 222, 223 and 22—KH 214 medium contains 36.4 g of paste of Salmon fish with 720 mL of WFI. Sample number 1, Tryptophan is added as a stabilizer.

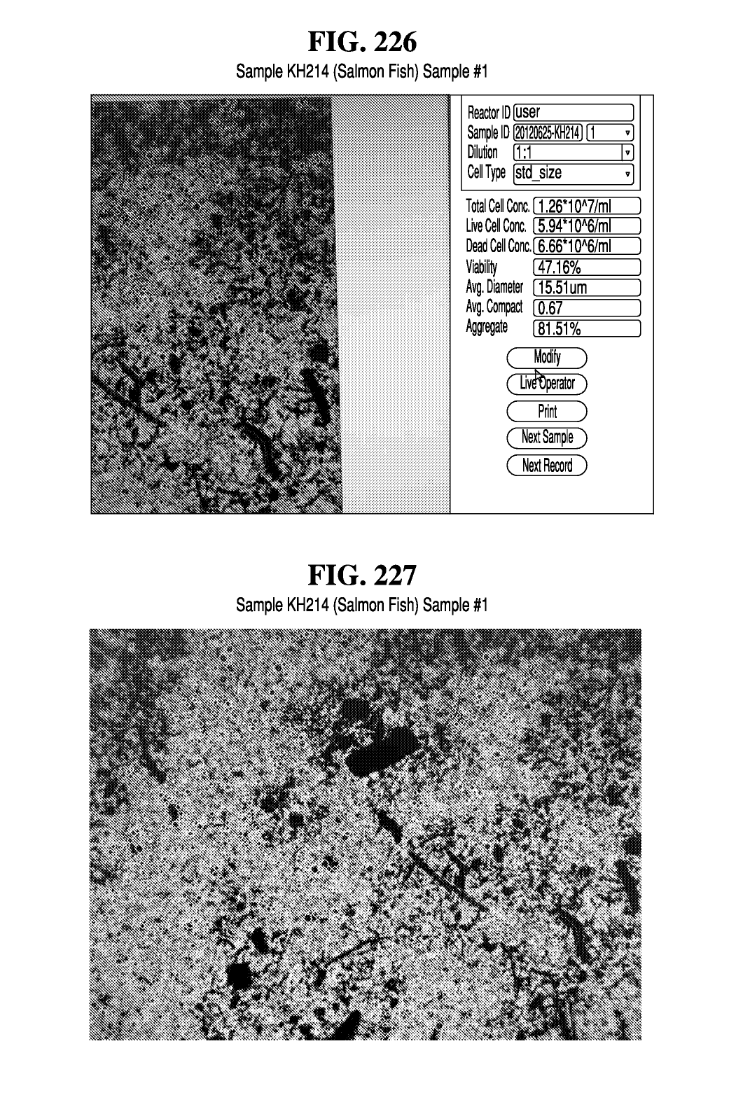

FIGS. 225, 226 and 22—KH214 medium sample number 2. Tryptophan is added as a stabilizer.

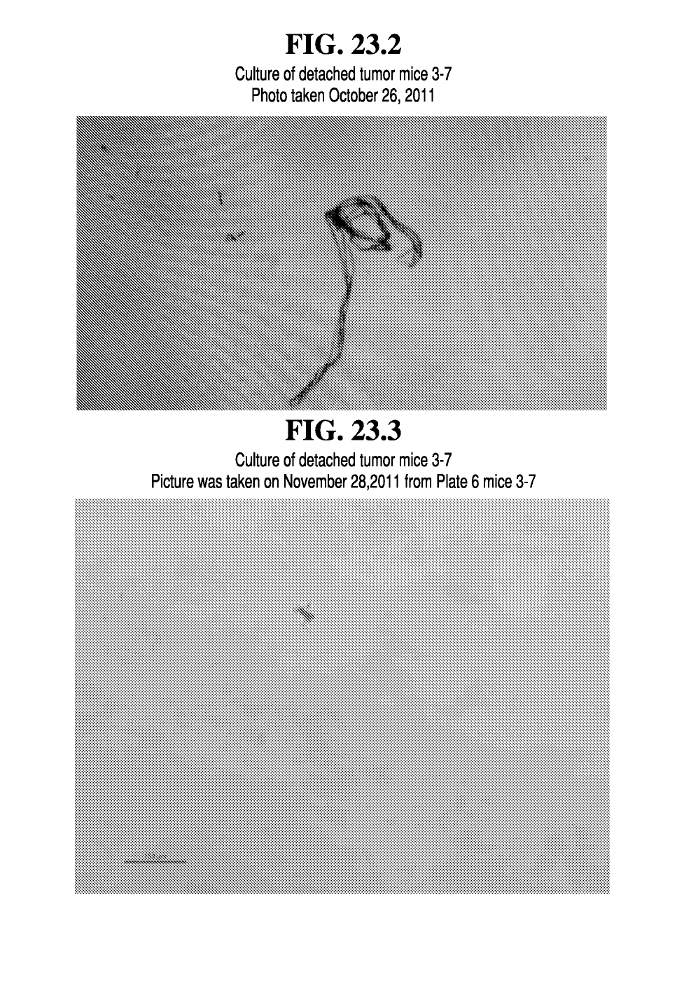

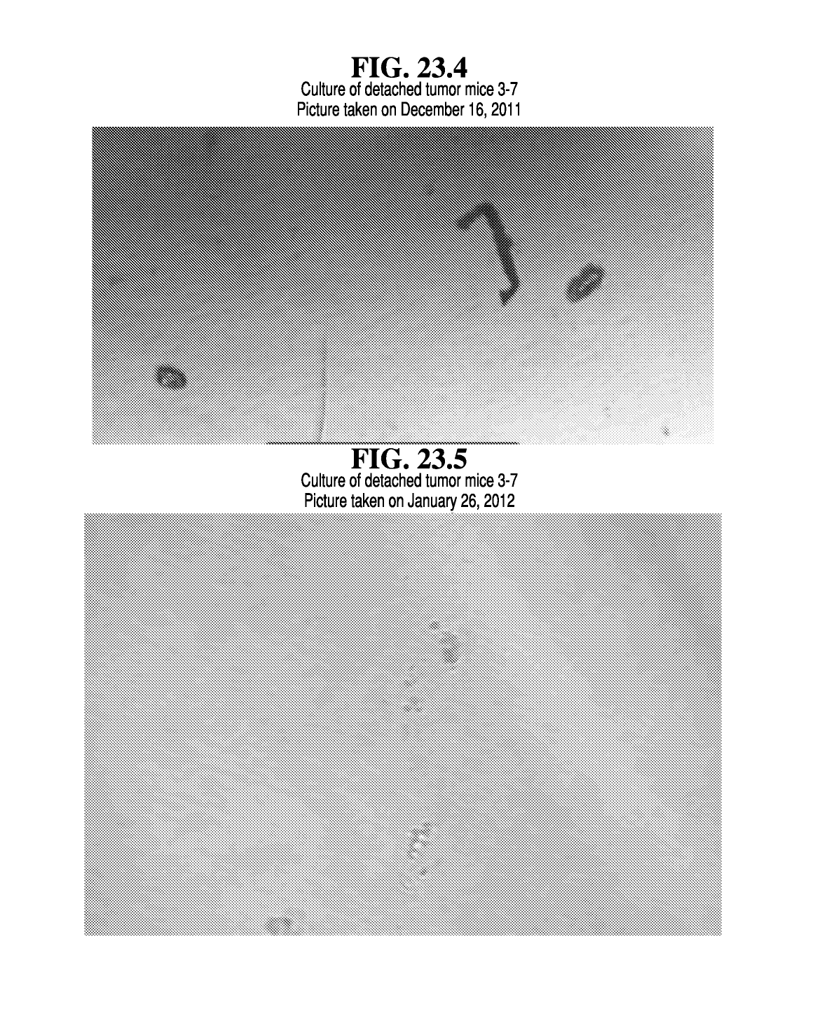

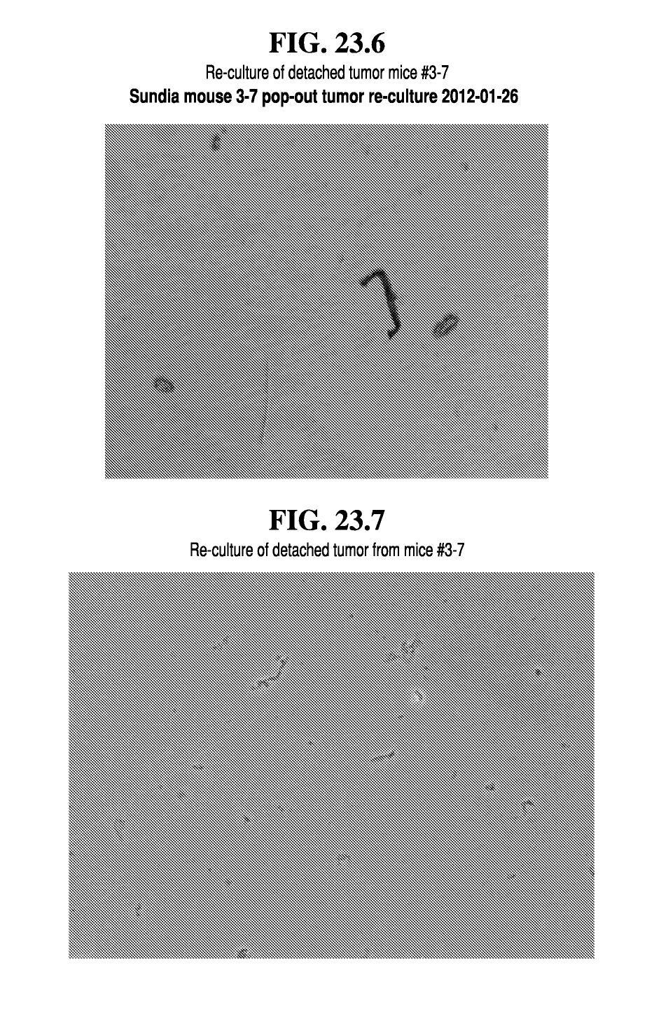

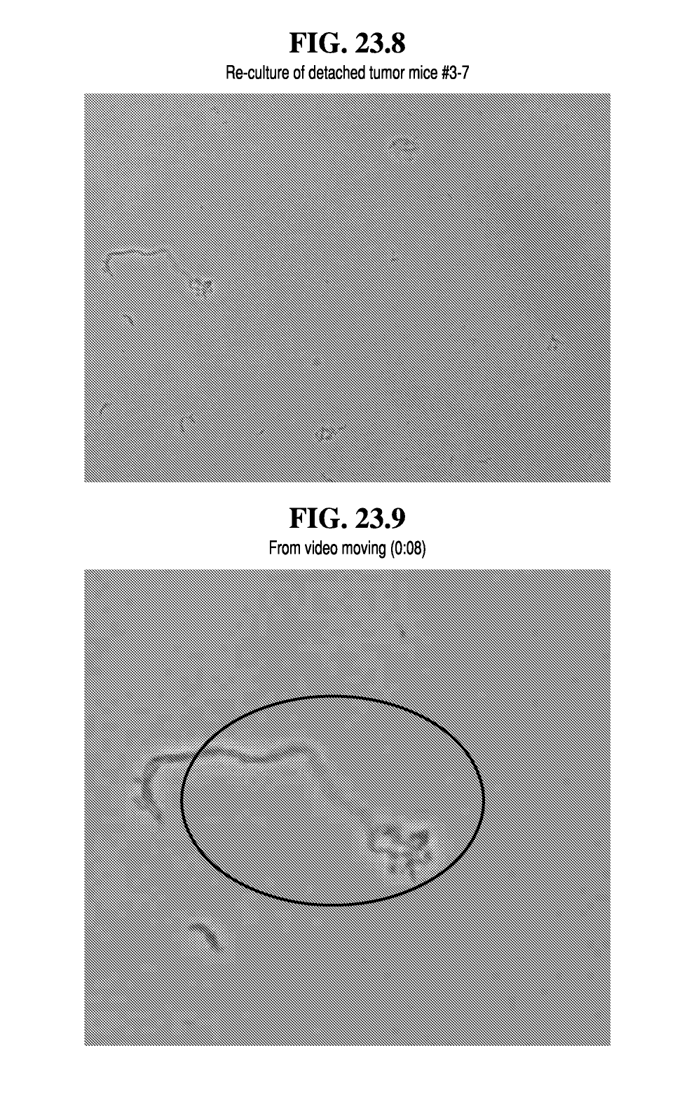

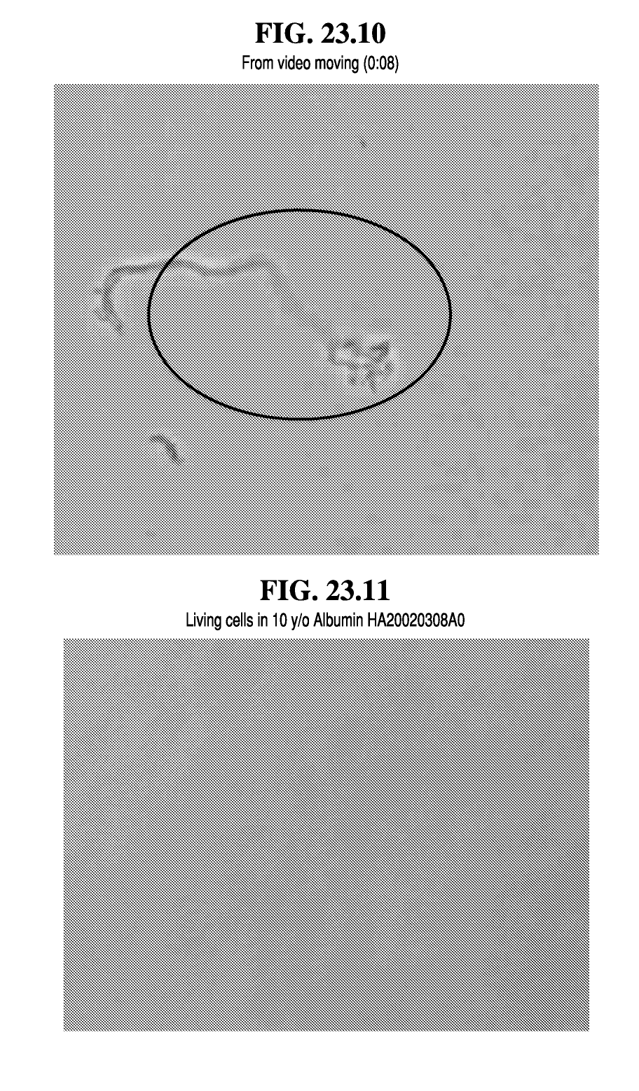

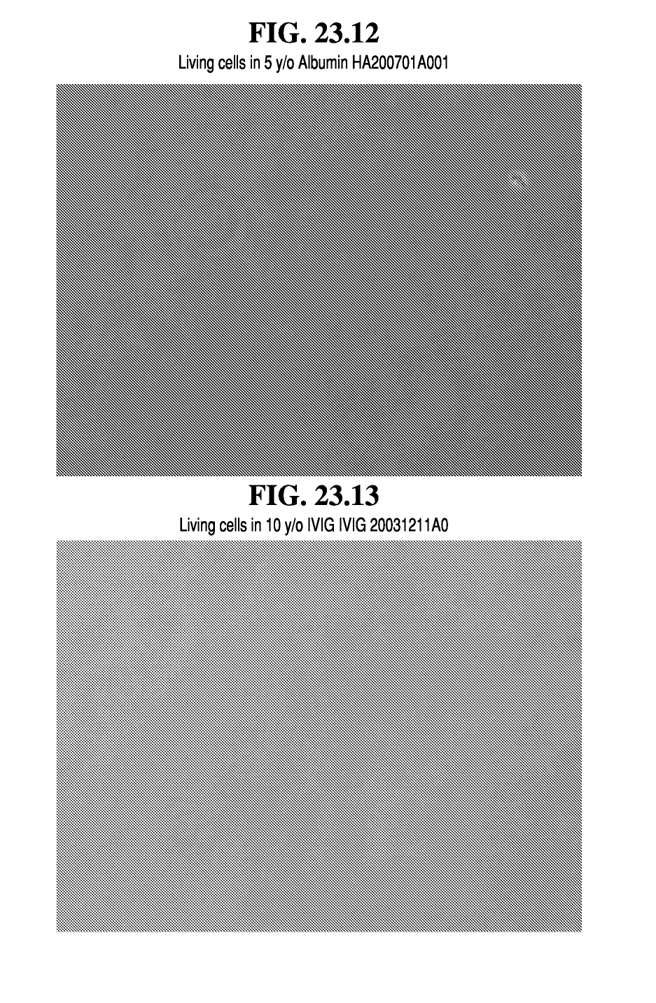

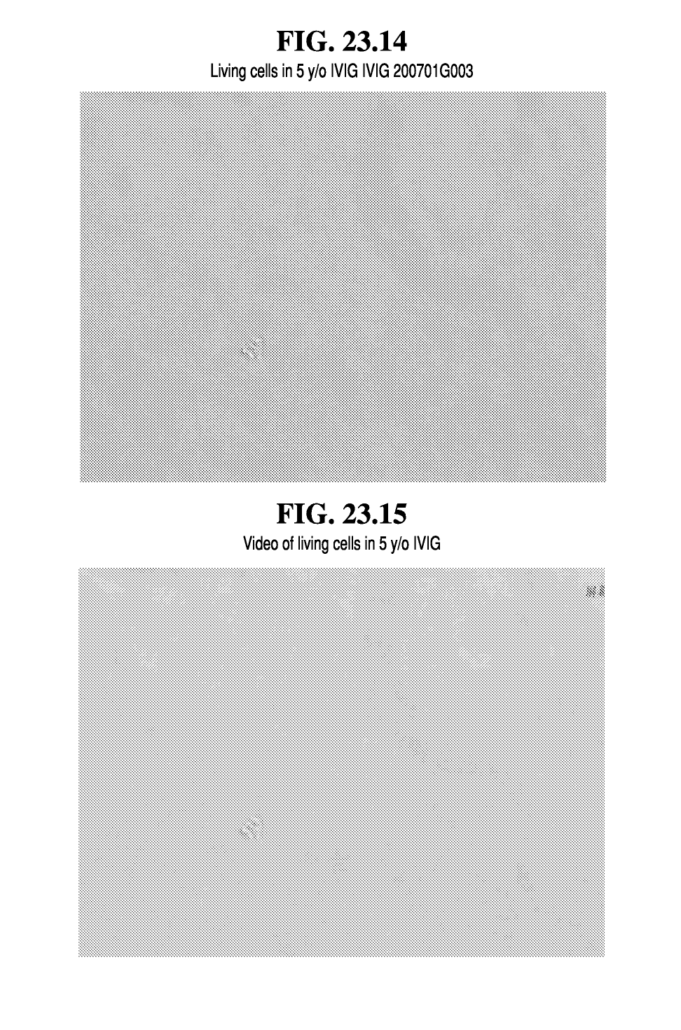

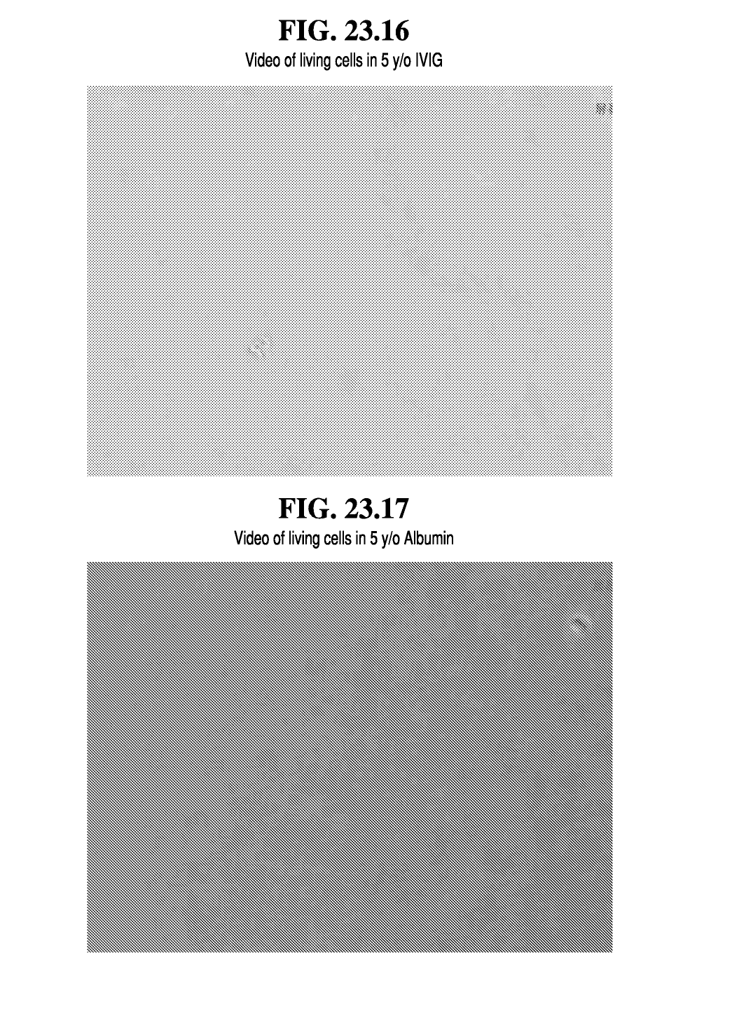

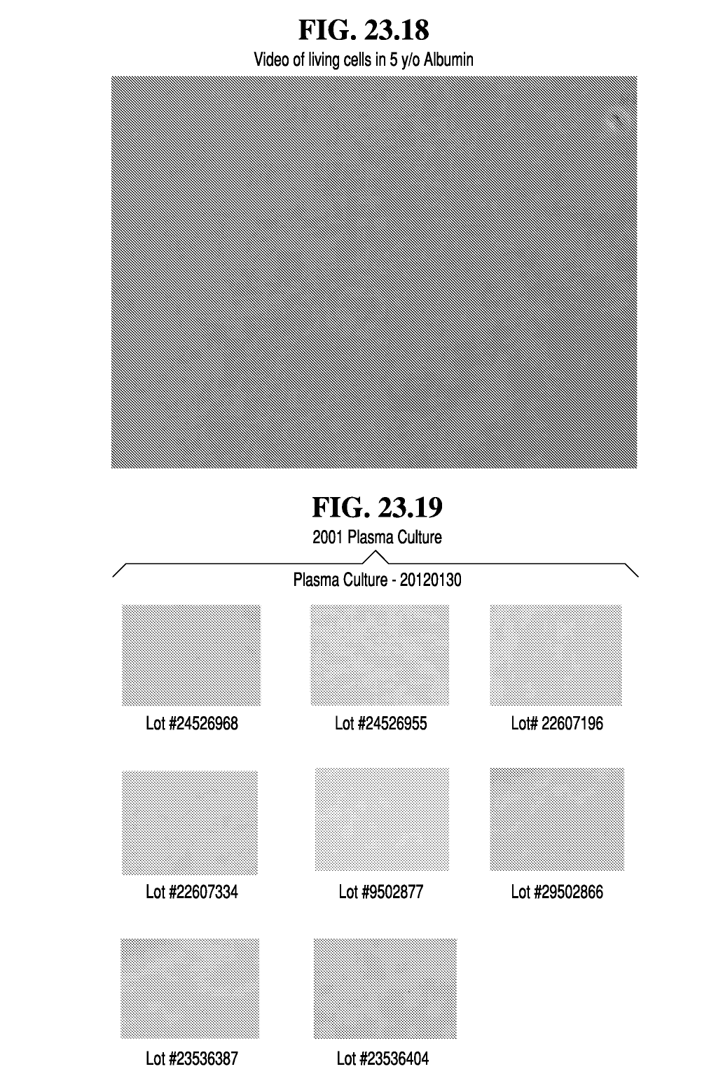

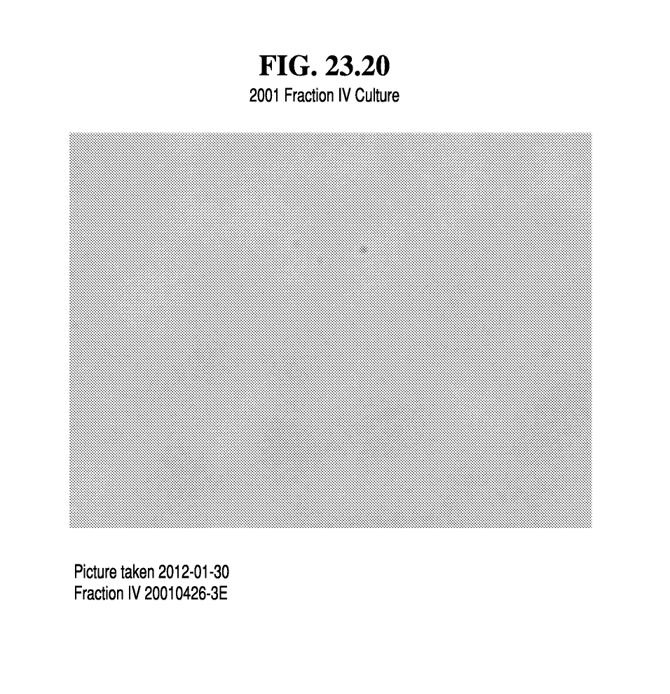

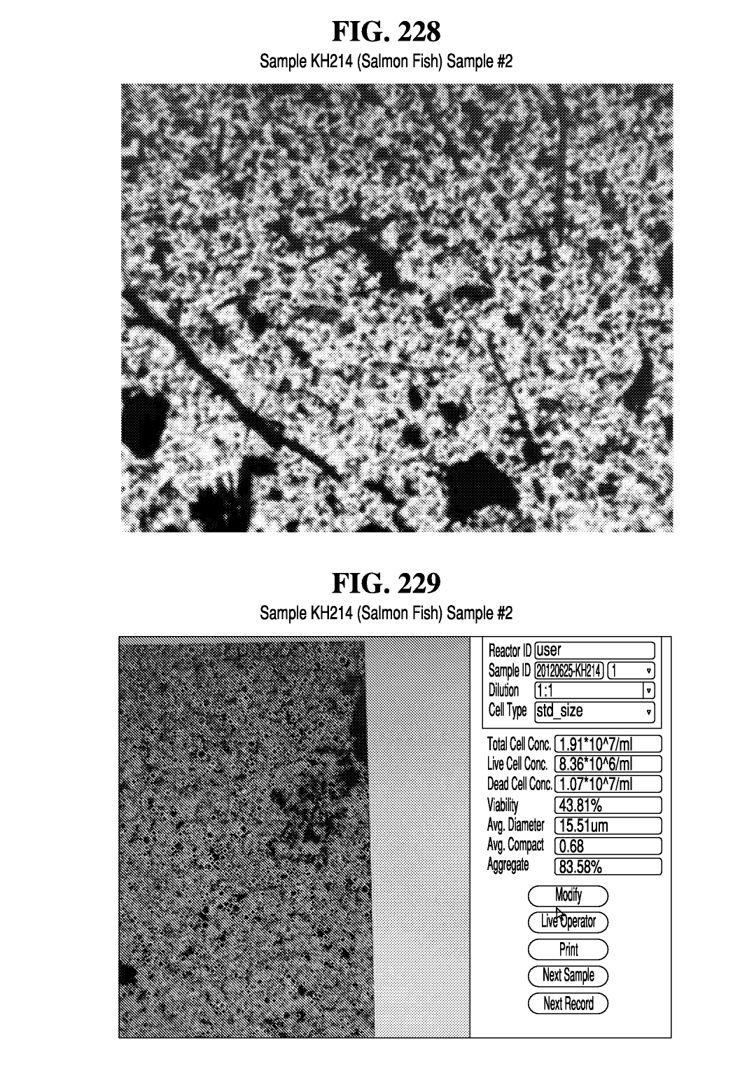

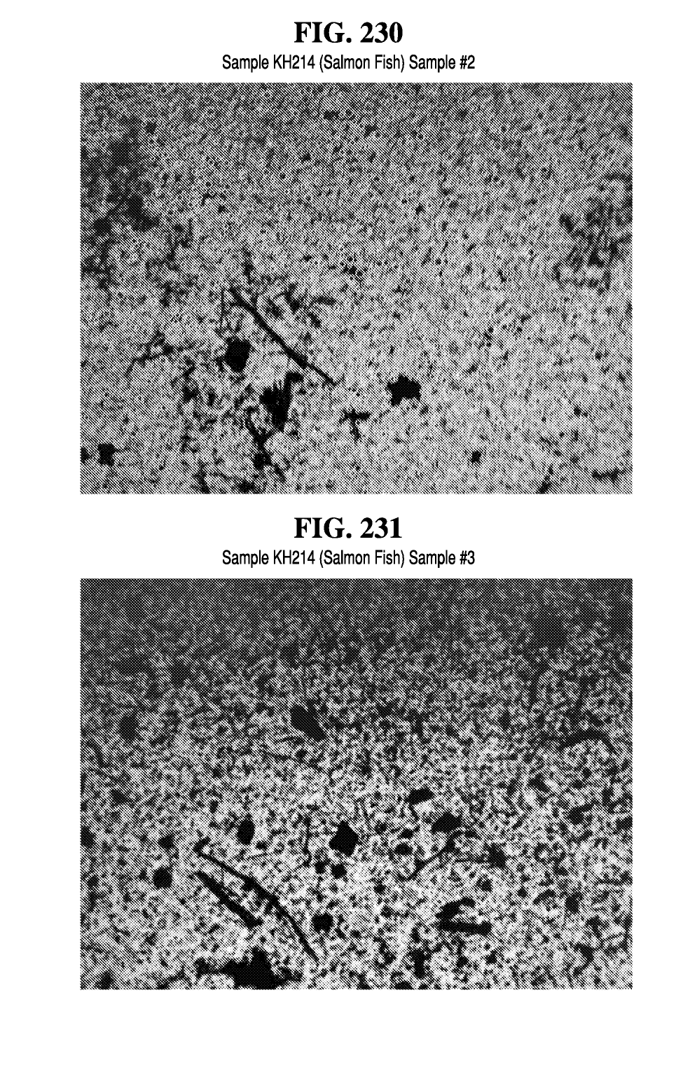

FIGS. 228, 229 and 23—KH214 medium sample number 3. Tryptophan is added as a stabilizer.

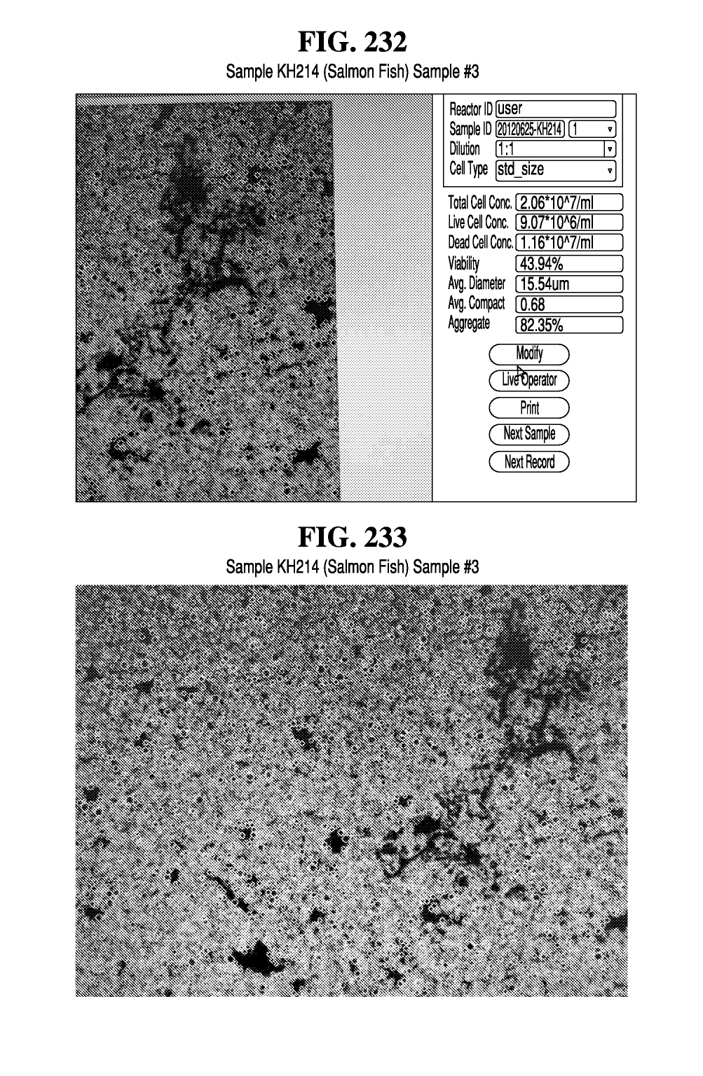

FIGS. 231, 232 and 23—KH214 medium sample number 4. Without Tryptophan.

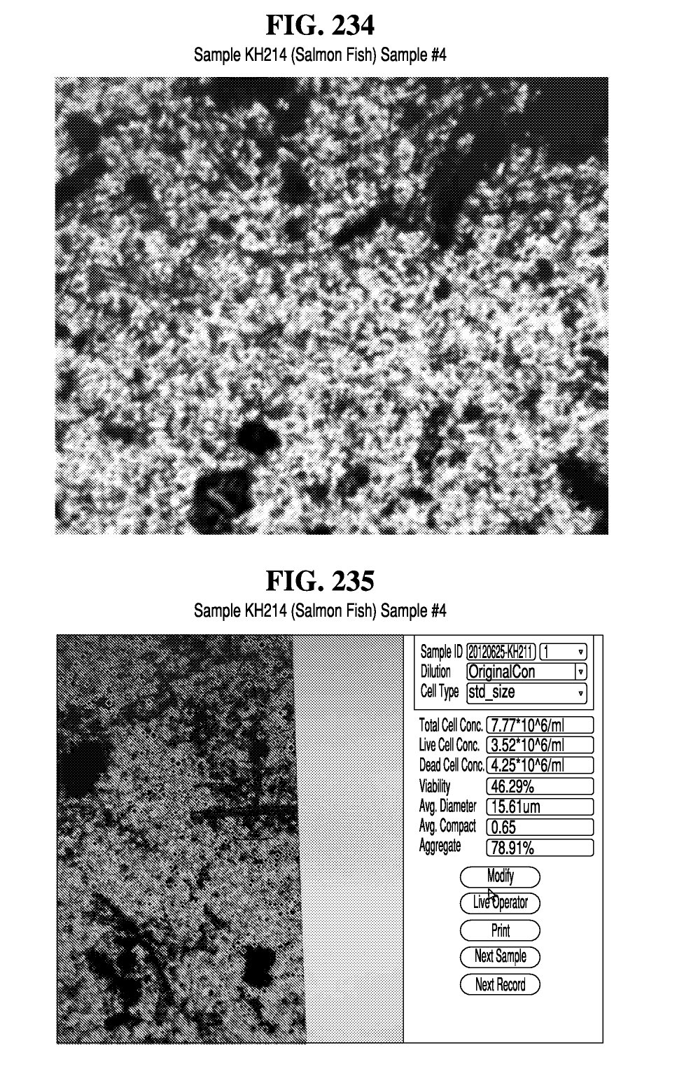

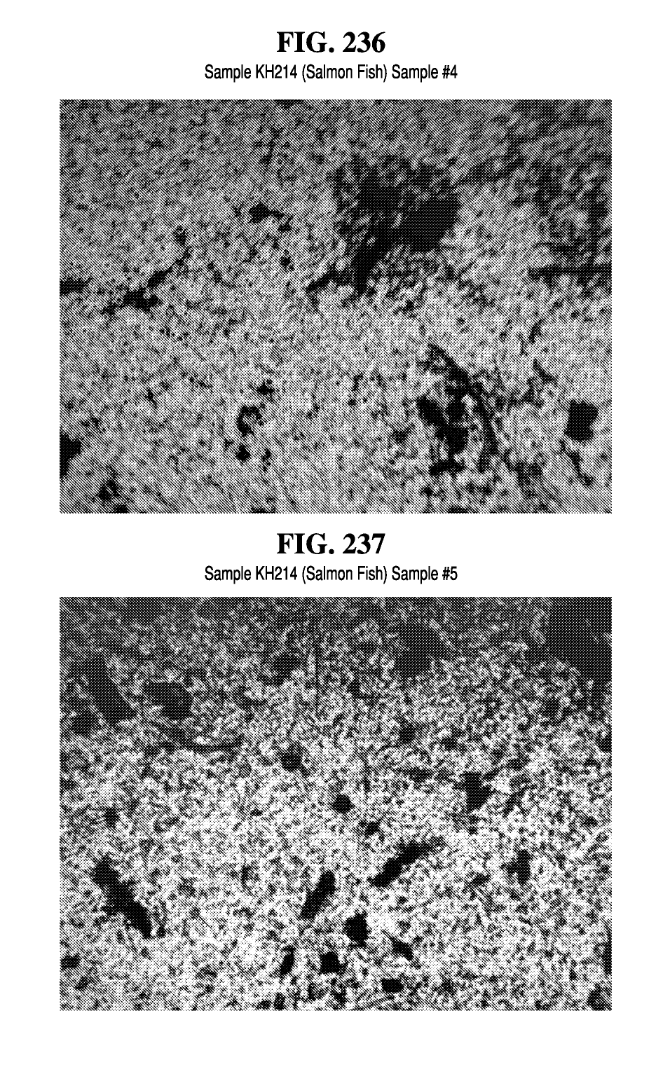

FIGS. 234, 235 and 23—KH214 medium sample number 4. Without Tryptophan.

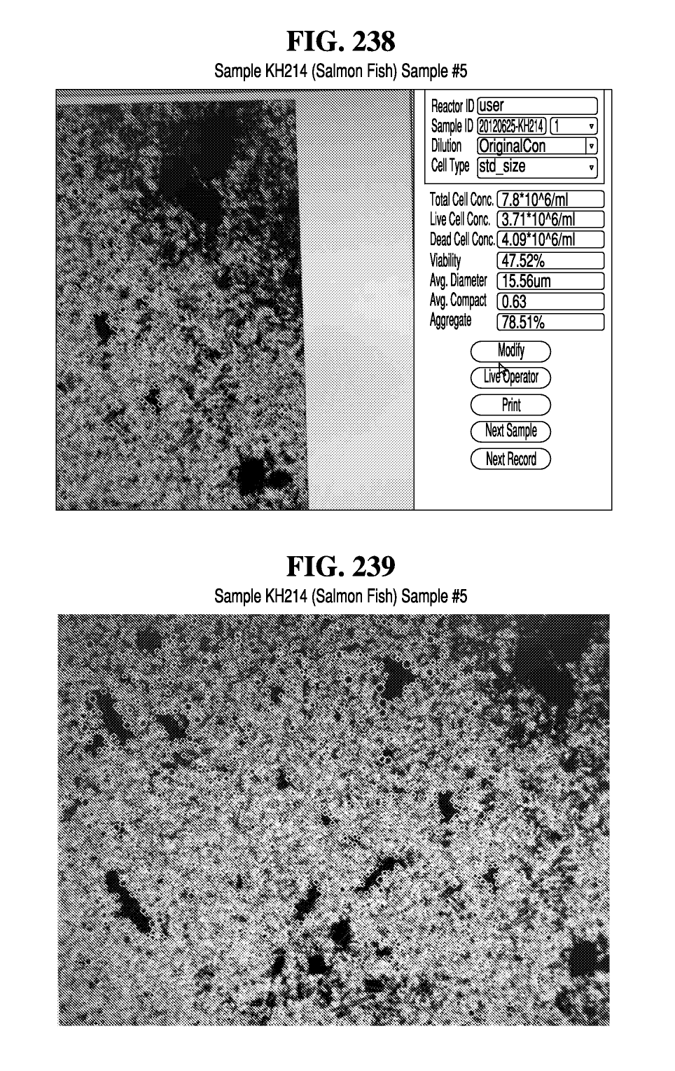

FIGS. 237, 238 and 23—KH301 medium sample of Chinese yam (1 tablet in 15 mL of Water for Injection)

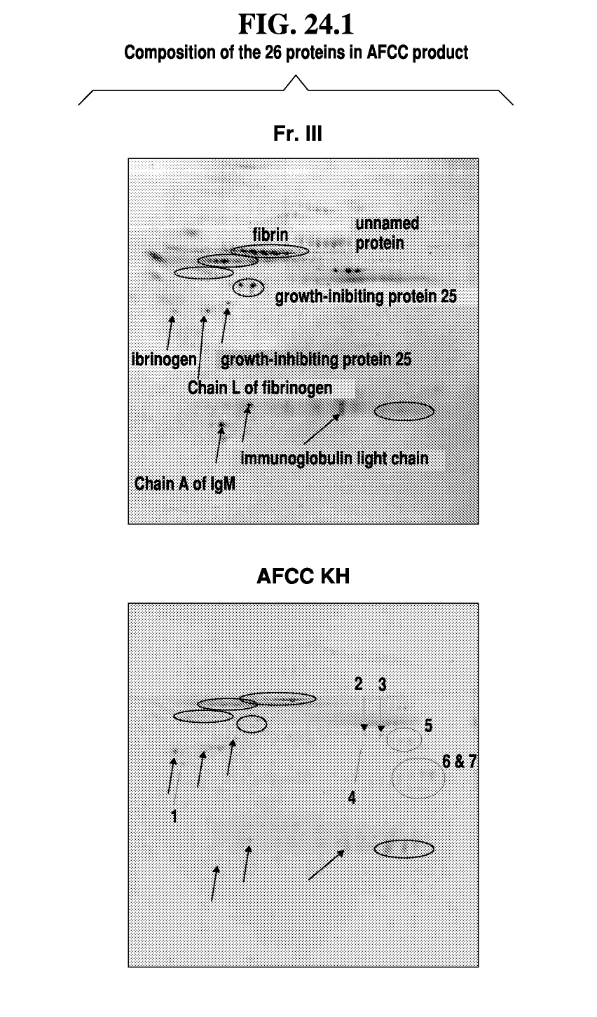

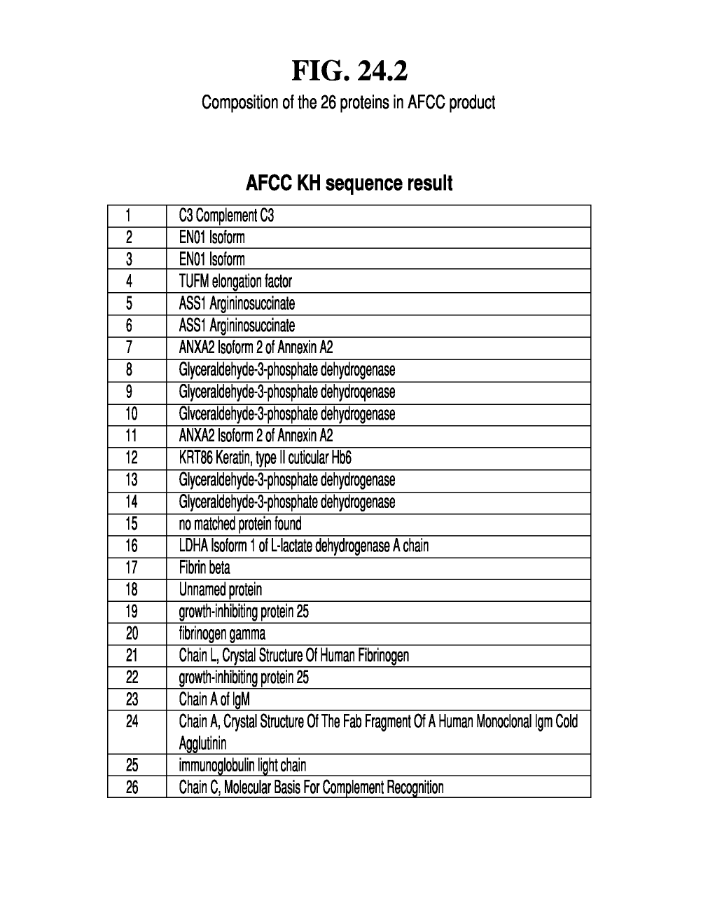

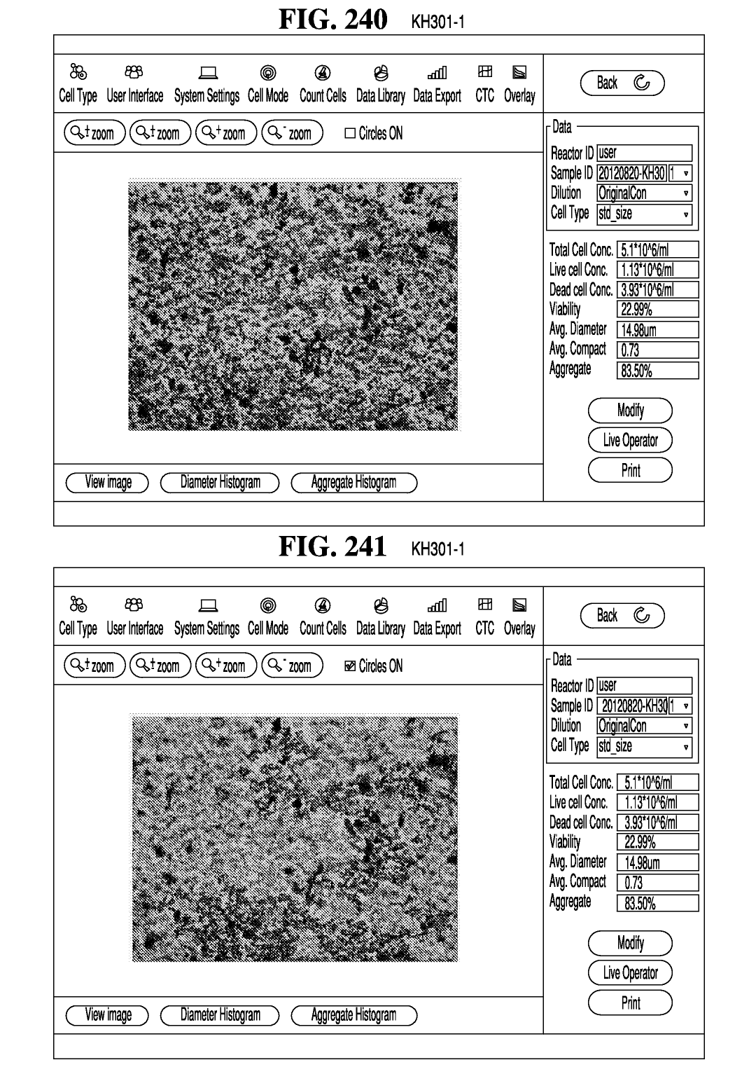

FIGS. 240 and 24—KH302 medium sample of Chinese worm medicine (Dong Chong Xia Cao)

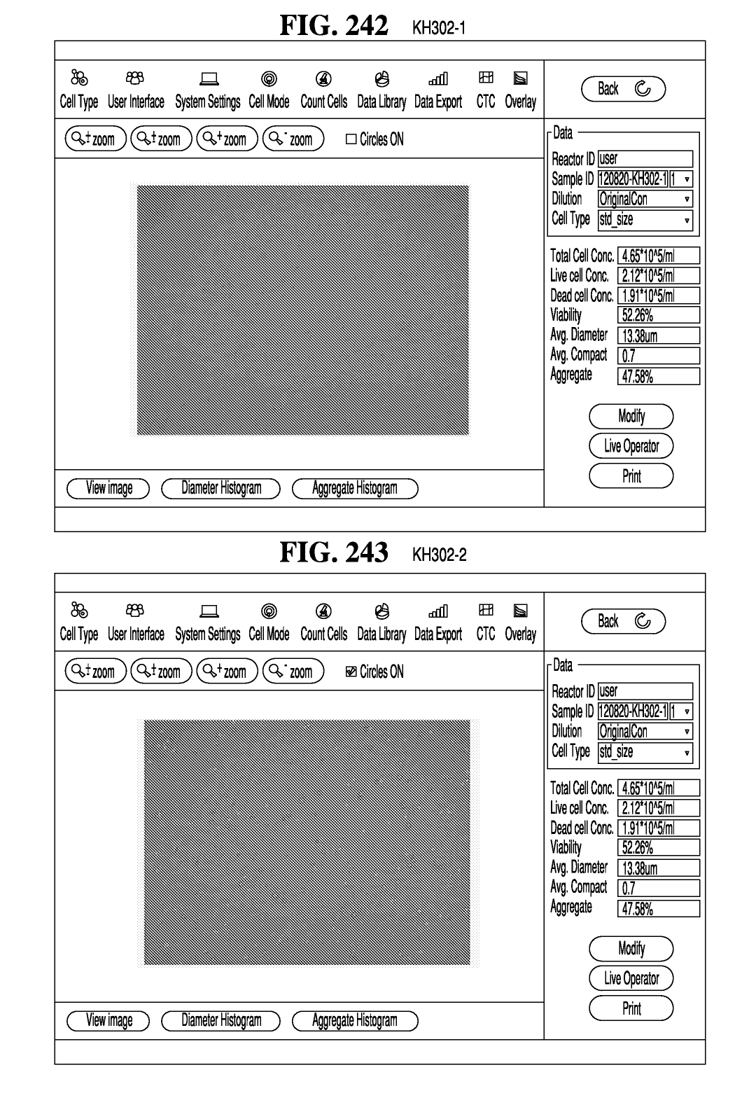

FIGS. 242 and 24—KH303 medium sample of Tibet Leaves

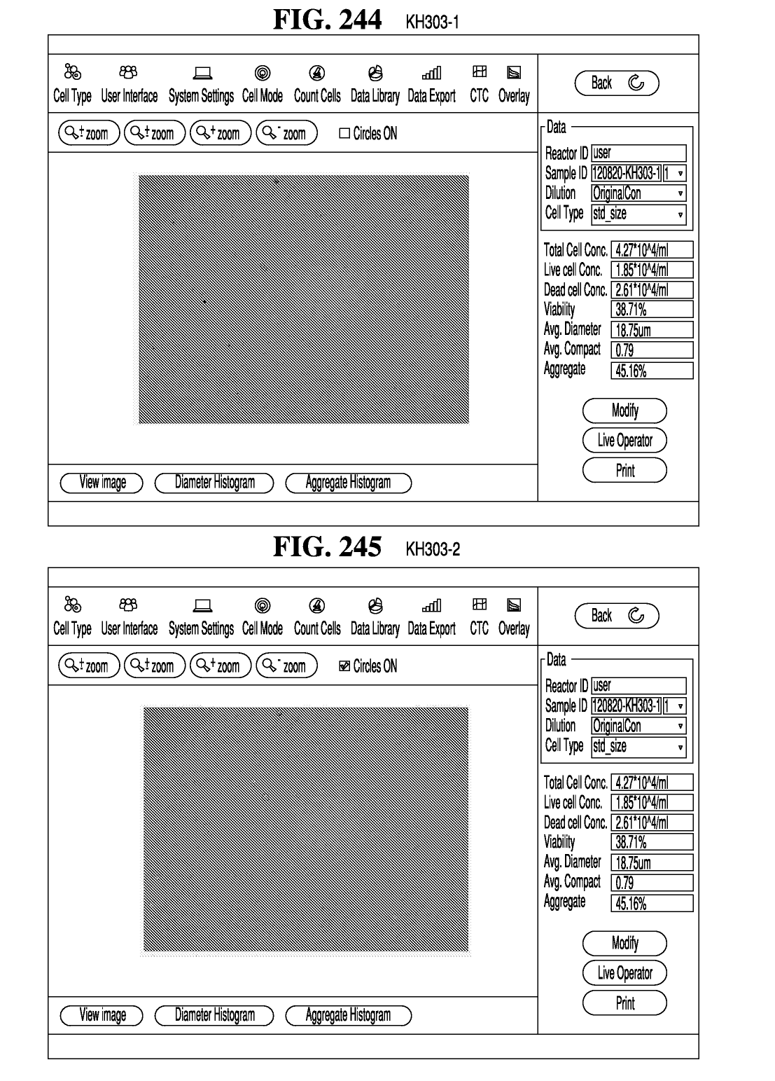

FIGS. 244 and 24—KH304 medium sample of Bovine Milk for new born baby

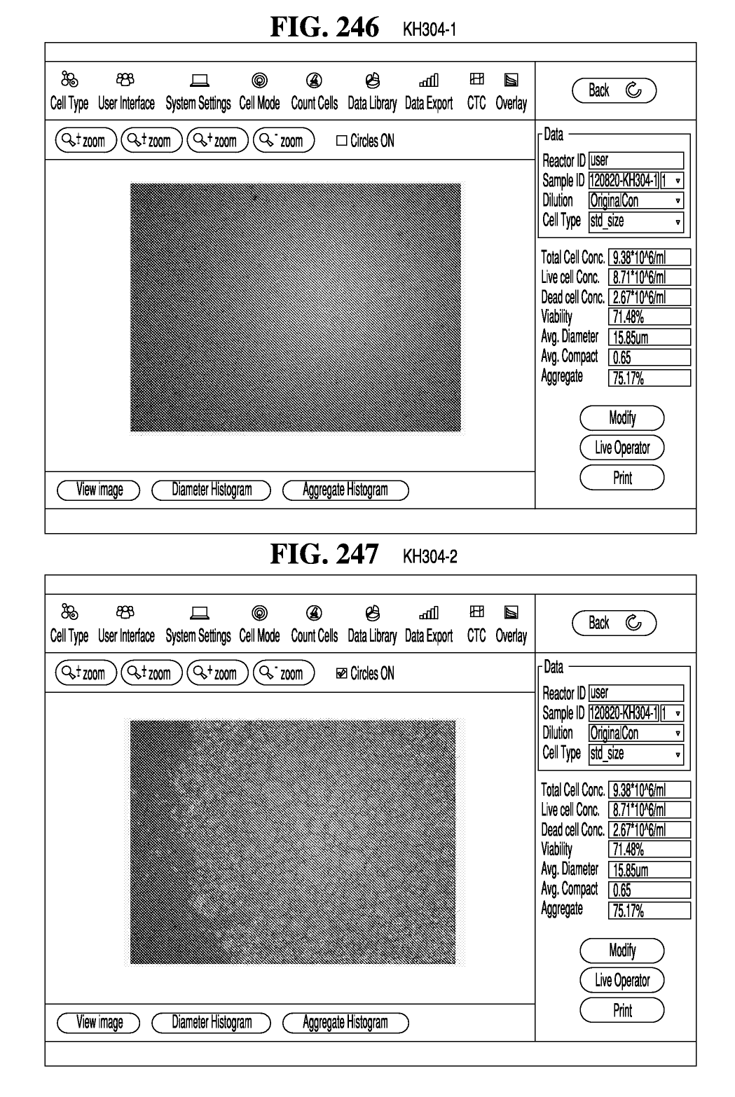

FIGS. 246 and 24—KH305 medium sample of Bovine Milk for three month old baby

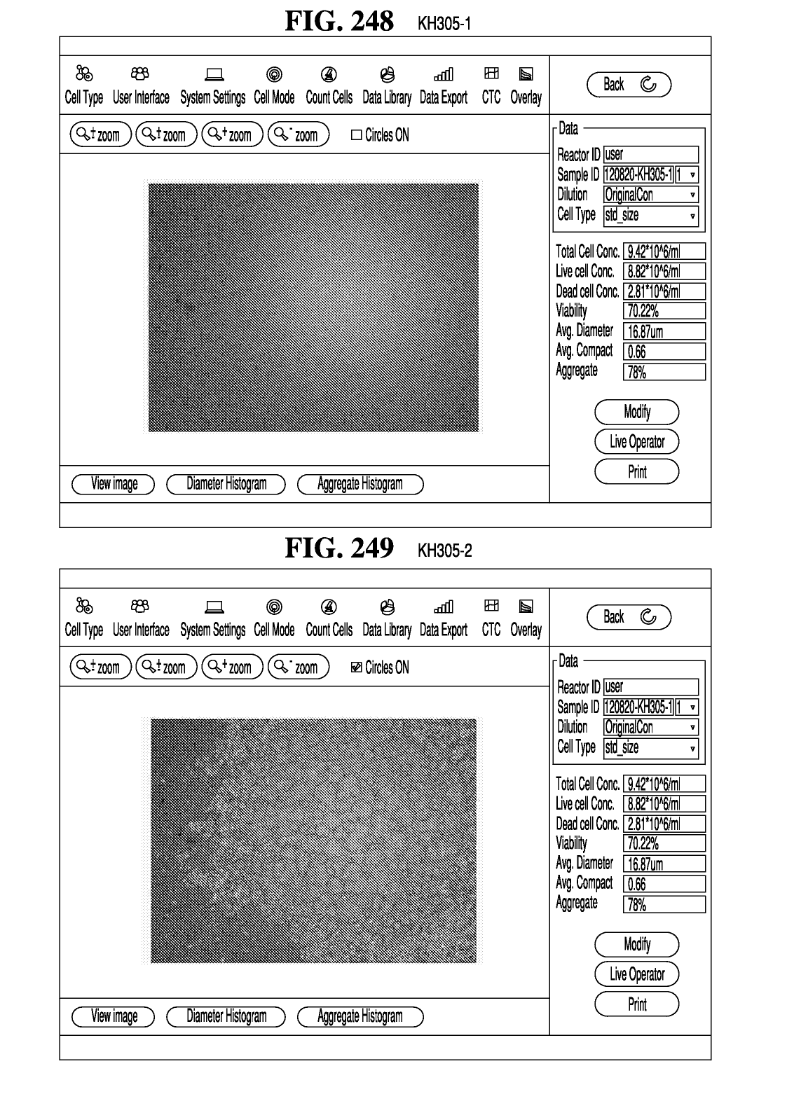

FIGS. 248 and 24—KH306 medium sample of Bovine Milk for six month old baby

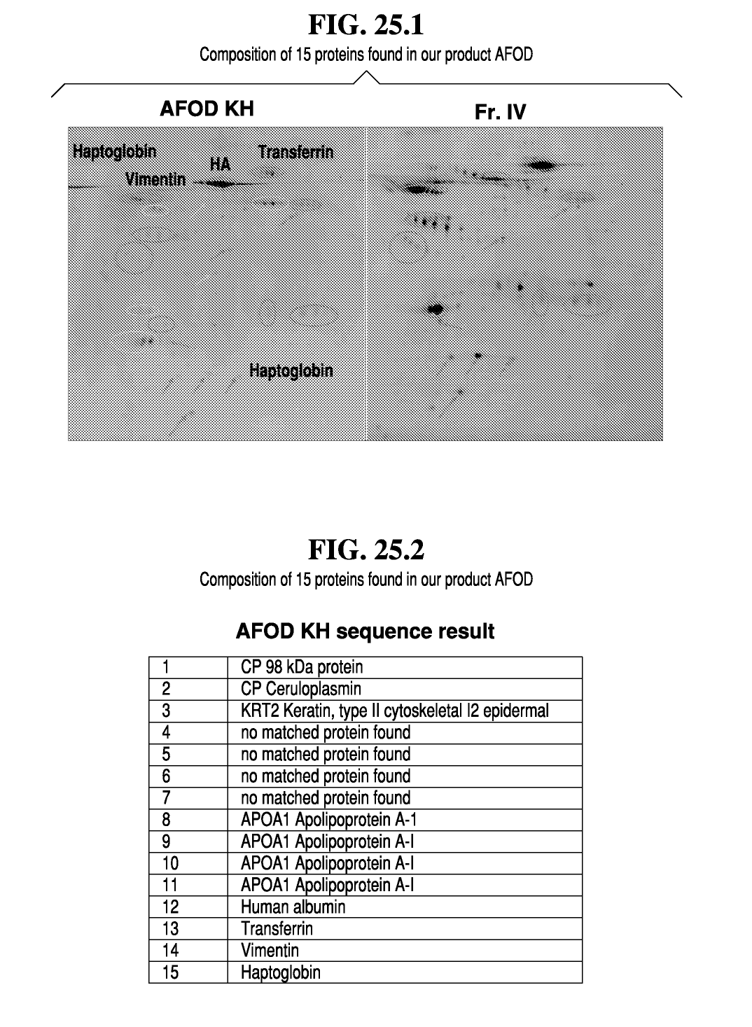

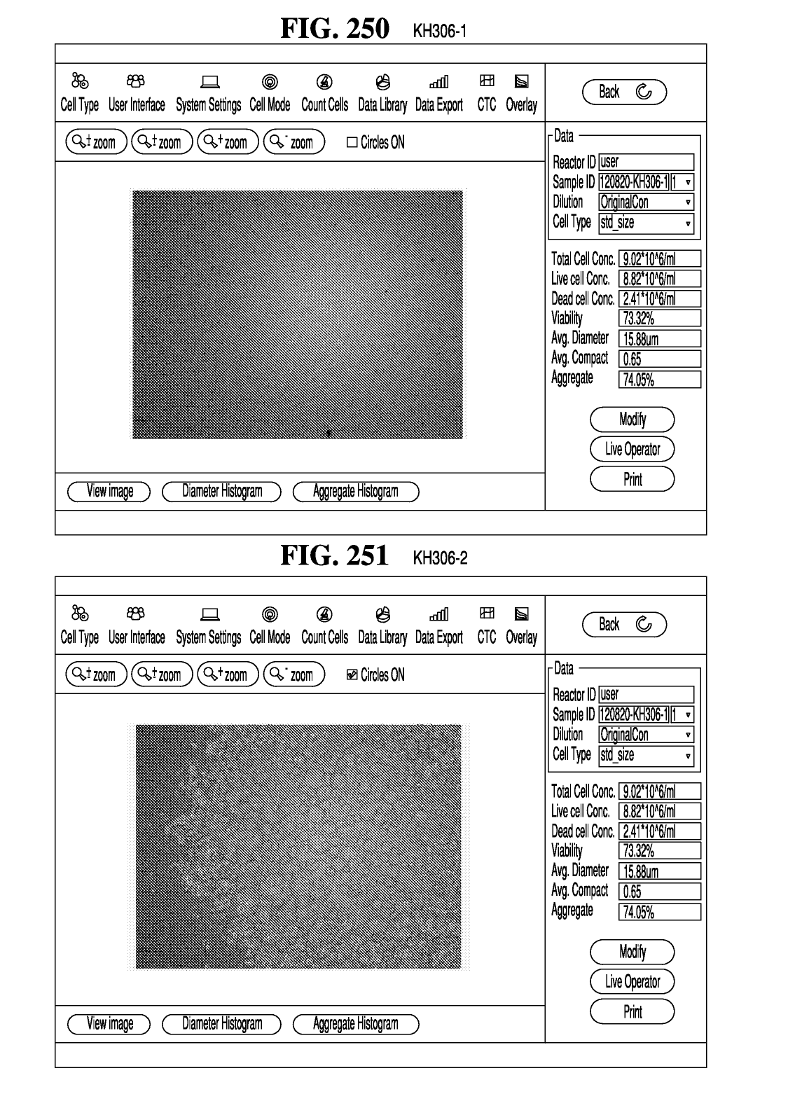

FIGS. 250 and 25—KH307 medium sample of Bovine Milk for 1 year old baby

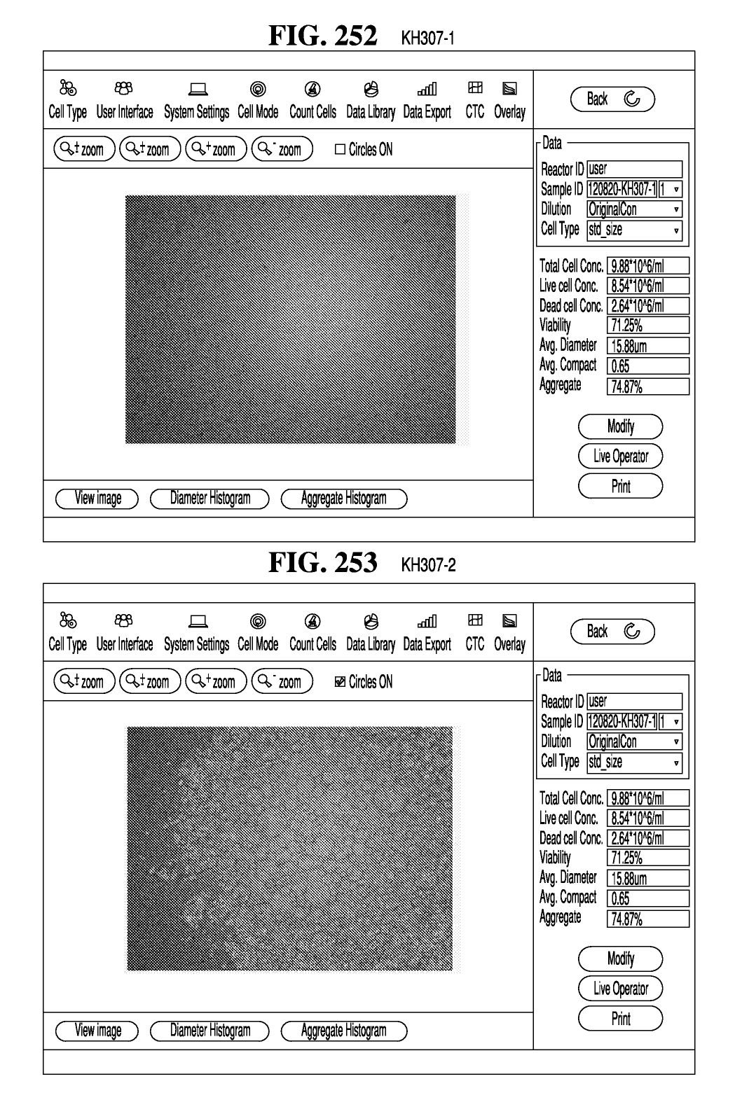

FIGS. 252 and 25—KH308 medium sample of Bovine Milk

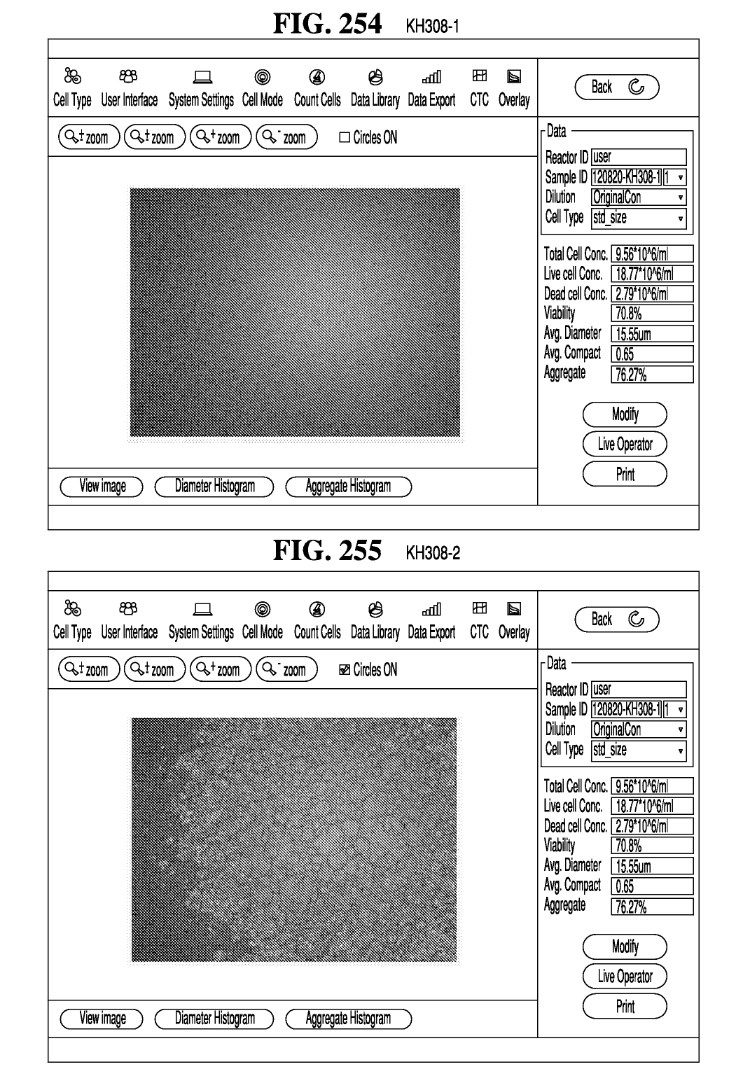

FIGS. 254 and 25—KH309 medium sample of Human Placenta

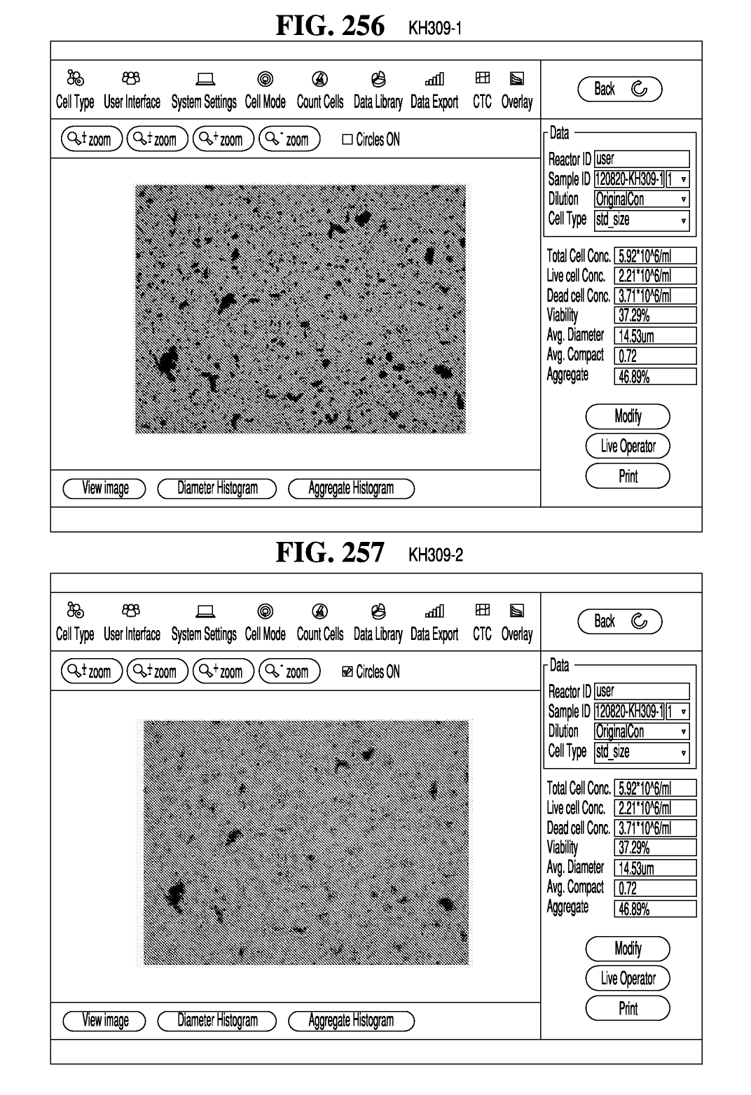

FIGS. 256 and 25

### IN Vitro Studies

Inflammation Markers
Cancer cells vs KH100-KH129 mediums
Characterization of cultured cells
Quantification of cholesterol and Triglyceride levels in RAAS products (2011)
Quantification of cholesterol and Triglyceride levels in RAAS products (2012)

### Inflammation Markers

The study has been performed by the school of pharmacy of Fudan University in Shanghai, China. 50 rabbits were used to study the efficacy of AFOD RAAS 1® (APOA1) for atherosclerosis and the inflammation.

MMP2 belongs to Proteins of the matrix metalloproteinase (MMP) family, which is involved in the breakdown of extracellular matrix in normal physiological processes, such as embryonic development, reproduction, and tissue remodeling, as well as in disease processes, such as arthritis and metastasis. Most MMP's are secreted as inactive proproteins which are activated when cleaved by extracellular proteinases. MMP2 degrades type IV collagen, the major structural component of basement membranes. It plays a role in endometrial menstrual breakdown, regulation of vascularization and the inflammatory response. The increase of MMP2 means the increase of inflammation response. Decrease represents the alleviation of inflammation.

PPAR (peroxisome proliferator-activated receptors Peroxisome proliferator-activated receptors) is a family of the nuclear hormone receptors, including 3 ligand-activated transcription factors: PPARalpha (NR1C1), PPARbeta/delta (NUC1; NR1C2), and PPARgamma (NR1C3). PPARalpha, -beta/delta, and -gamma are encoded by different genes but show substantial amino acid similarity, especially within the DNA and ligand binding domains. All PPARs act as heterodimers with the 9-cis-retinoic acid receptors (retinoid X receptor; RXRs) and play important roles in the regulation of metabolic pathways, including those of lipid of biosynthesis and glucose metabolism, as well as in a variety of cell differentiation, proliferation, and apoptosis pathways. Recently, there has been a great deal of interest in the involvement of PPARs in inflammatory processes. PPAR ligands, in particular those of PPARalpha and PPARgamma, inhibit the activation of inflammatory gene expression and can negatively interfere with pro-inflammatory transcription factor signaling pathways in vascular and inflammatory cells. The increased expression of PPARs helps in inhibiting the inflammation.

The NF-κB/Rel family includes NF-κB1 (p50/p105), NF-κB2 (p52/p100), p65 (RelA), RelB, and c-Rel (2). Most members of this family (RelB being one exception) can homodimerize, as well as form heterodimers with each other. The most prevalent activated form of NF-κB is a heterodimer consisting of a p50 or p52 subunit and p65, which contains transactivation domains necessary for gene induction. The expression of NF-κB proteins can provide site- and event-specificity in response to a particular stimulus. NF-κB is clearly one of the most important regulators of proinflammatory gene expression. Synthesis of cytokines, such as TNF-α, IL-1β, IL-6, and IL-8, is mediated by NF-κB, as is the expression of cyclooxygenase 2 (Cox-2). The increased expression of NF-kB increase the inflammatory response.

COX-2 is undetectable in most normal tissues. It is an inducible enzyme, becoming abundant in activated macrophages and other cells at sites of inflammation. COX-2 which is associated with pain and inflammation. However there is studies showing that COX-2 is associated with an inflammatory reaction during the early phase of an inflammatory response (at about 2 hours), later in the inflammatory process a swell of COX-2 exists which has been shown to have anti-inflammatory effects in the studied rats.

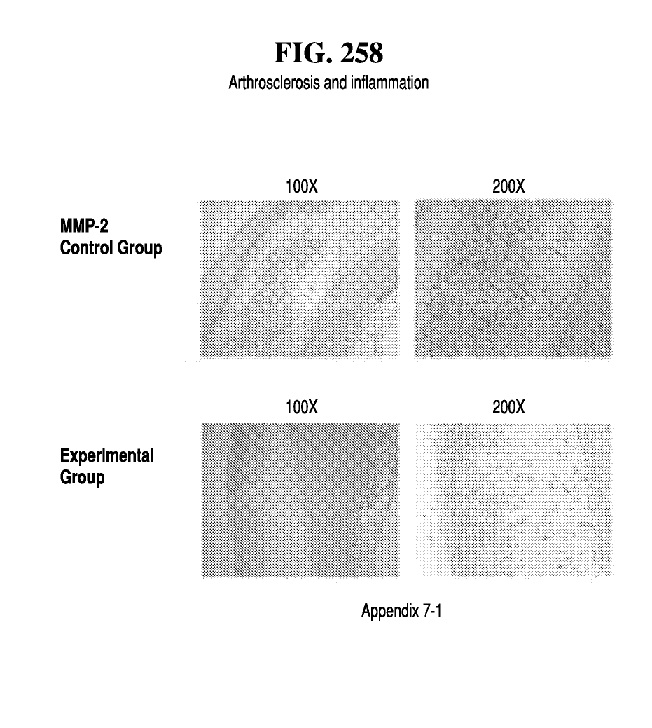

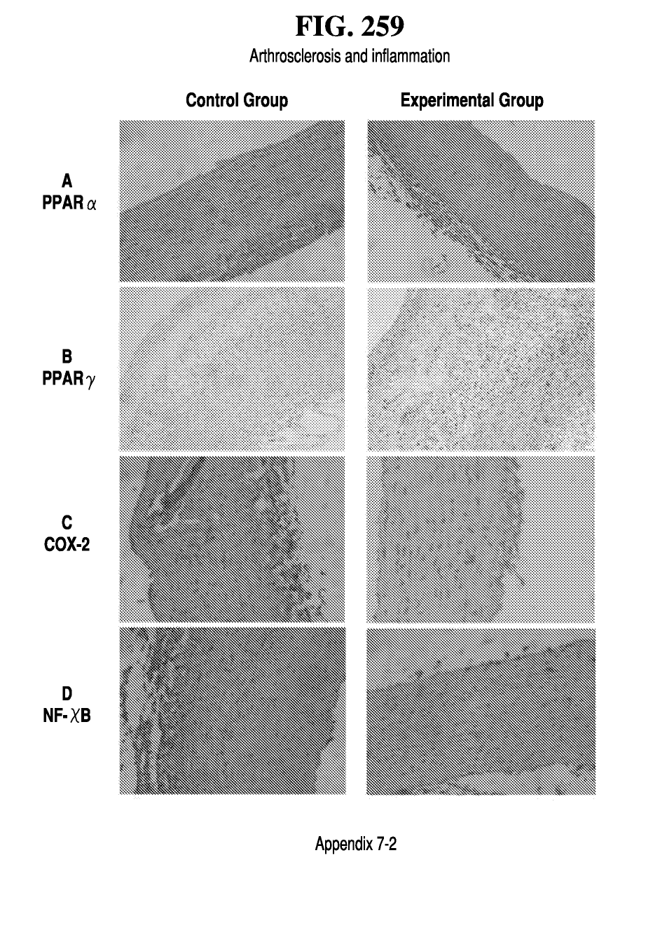

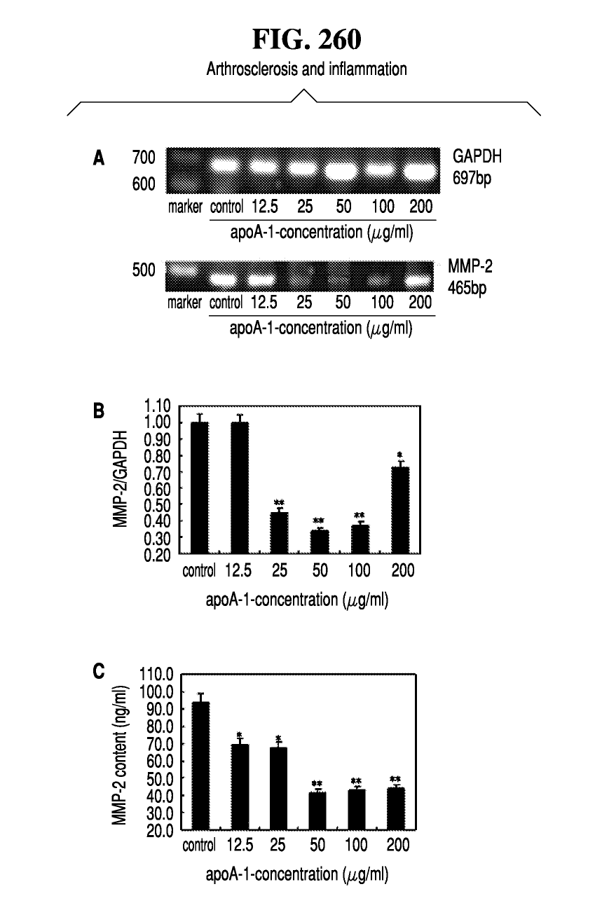

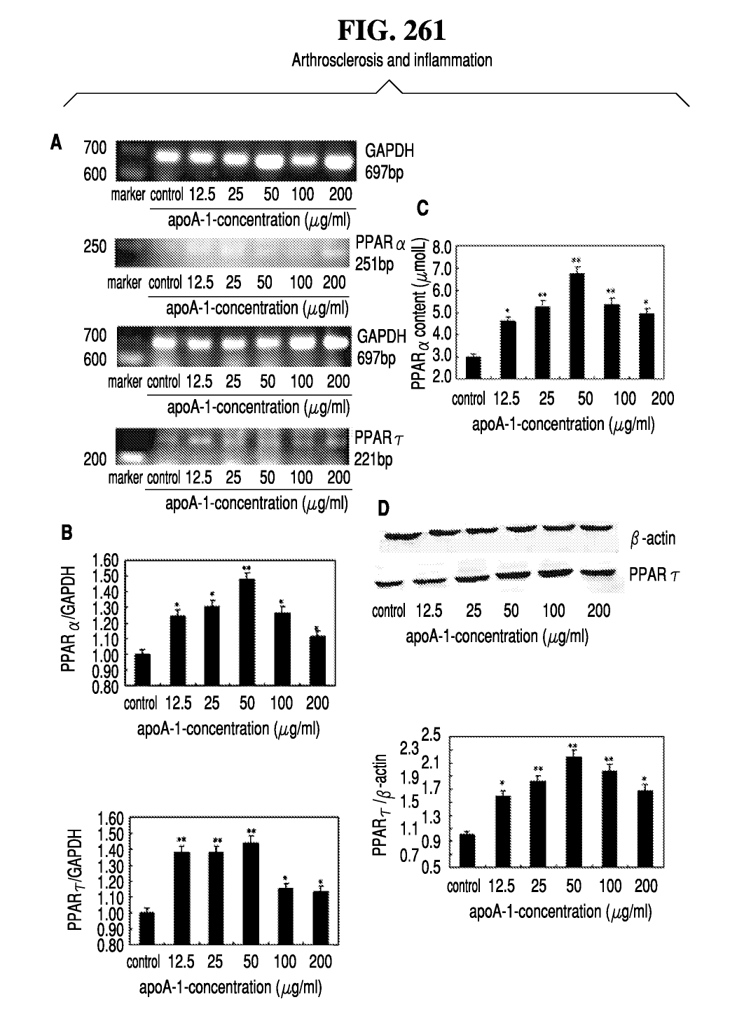

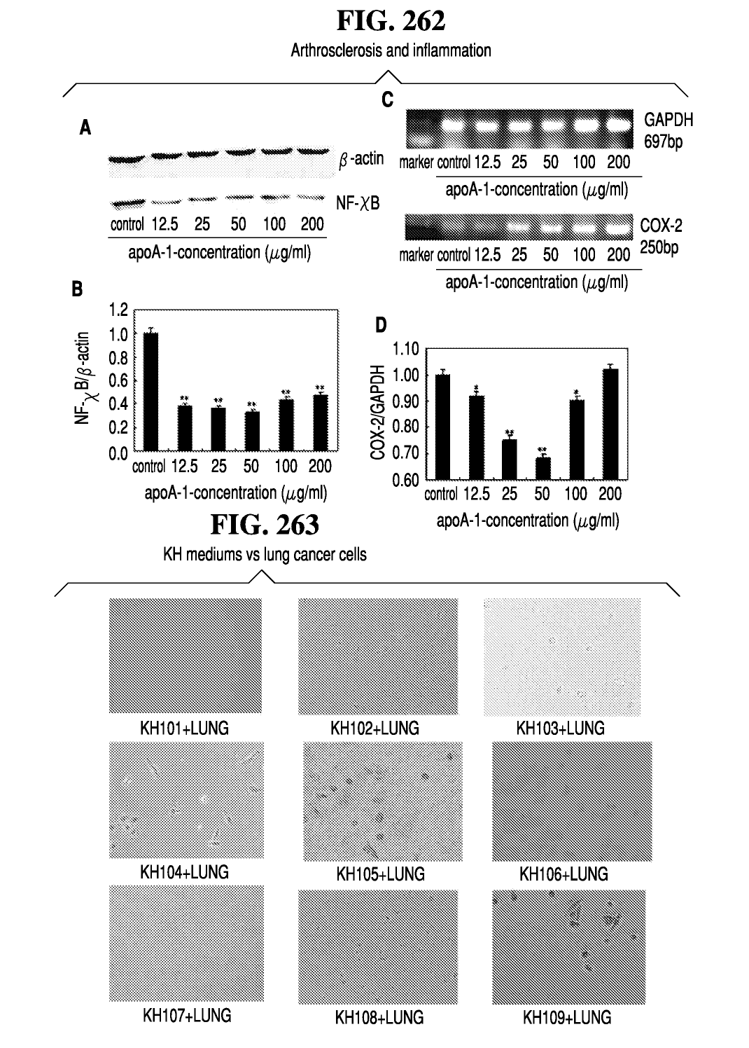

### FIGS. 258, 259, 260, 261 and 262

### Lung Cancer Cells vs KH100-KH129 Mediums

In order to find out if there is any effect on the cancer cell by using the KH mediums we have found that apparently the number of lung cancer cells have been reduced or completely blocked by KH101 which is from Non Sticky Rice. From which other companies in China have produced Human Albumin for use in the cell growth instead of feta bovine serum and also

Alpha 1 Antitrypsin is produced from Rice.

KH101 contains Albumin protein whose characteristic is the same as the Human Albumin therefore it is possible that it may work and inhibit the growth of the cancer cells. Like in the case of our product following the process AFOD RAAS from the human plasma.

With regards to the animal we have found that the bovine human albumin, bovine immunoglobulin, pig thrombin and pig fibrinogen have also inhibited lung cancer cell growth. In this case KH205 medium which consists of Pork meat shows inhibition of the lung cancer cell growth. Same is the case with the KH206 medium which consists of Beef meat.

Other mediums like egg yolk, egg white, crawfish and mackerel fish all show certain degree of inhibition of the lung cancer cells.

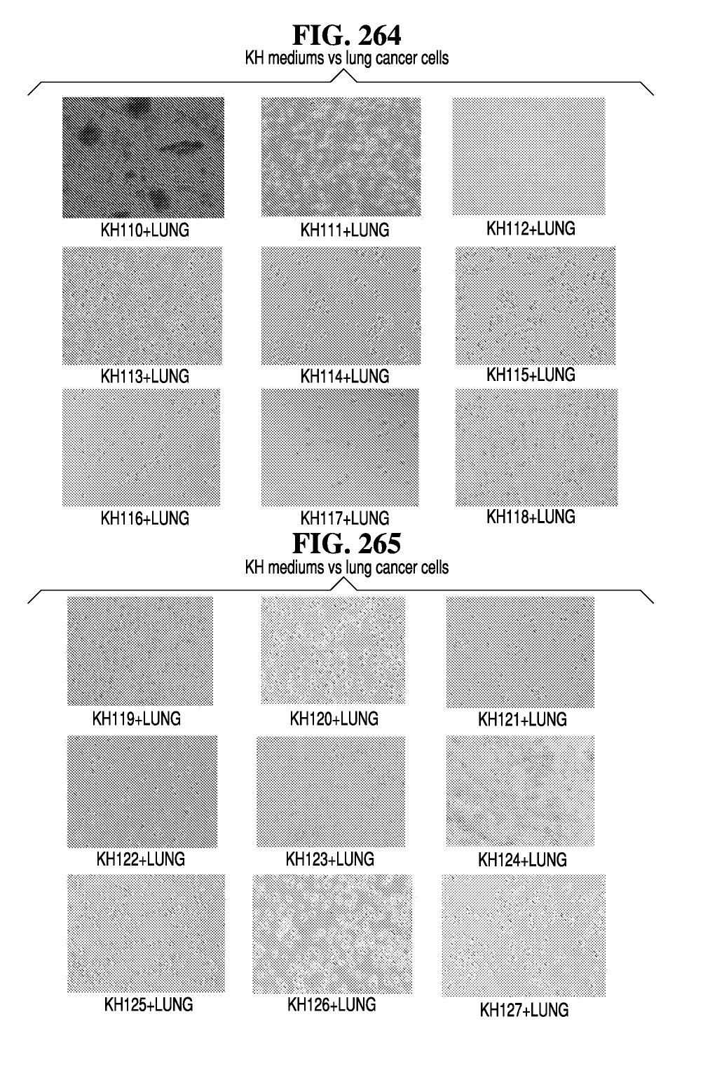

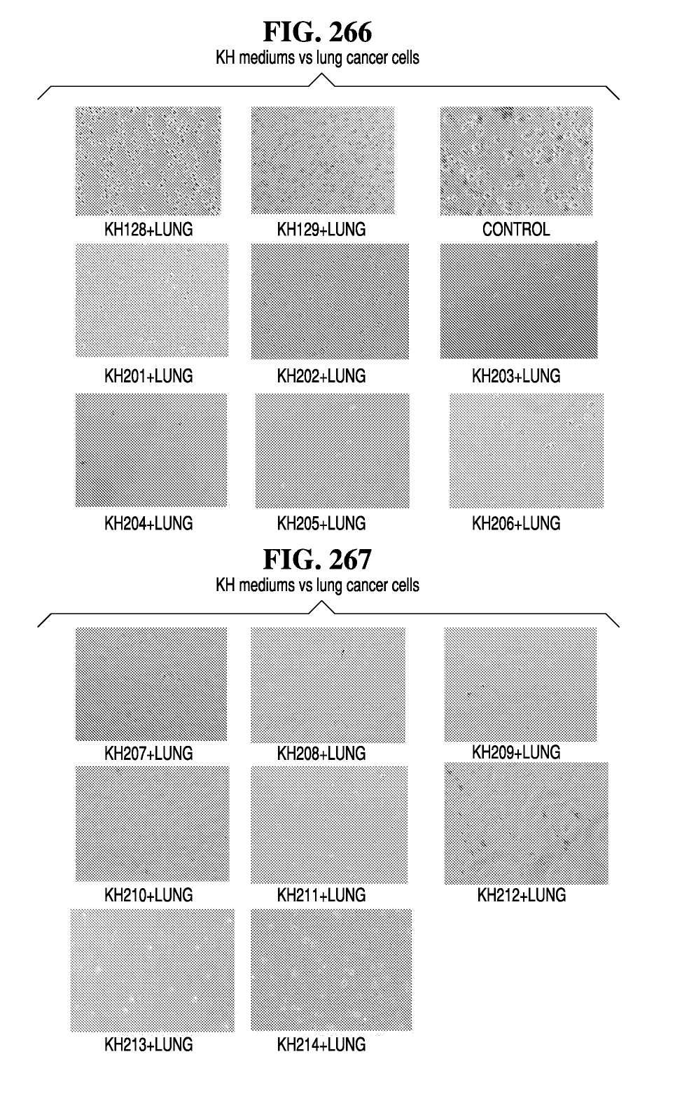

### FIGS. 263, 264, 265, 266, 267 and 268

There are plenty of undiscovered new KH cells in fruit, for example a grape which is the size of a finger nail contains 30,000 genes while a human being which weights average of 70 kilos has only 25,000 genes. In order to prove that there are new cells a number of plates containing cells have been analyzed and we found that the characterization of unknown cells to the CRO lab, but known to the inventor, for RAAS like the Dragon cell or other KH cells.

The above have been discovered in In-Vitro well study but not tested for CCK8 to measure the degree of inhibition of the cancer cells by each of the mediums. Such a study has been performed on Oct. 4, 2012 it is amazing to find out that all series of medium from KH100, KH200 and KH300 have different level of effect on the inhibition of the lung cancer, leukemia, gastric and breast both solid tumors and blood cancer have been tested.

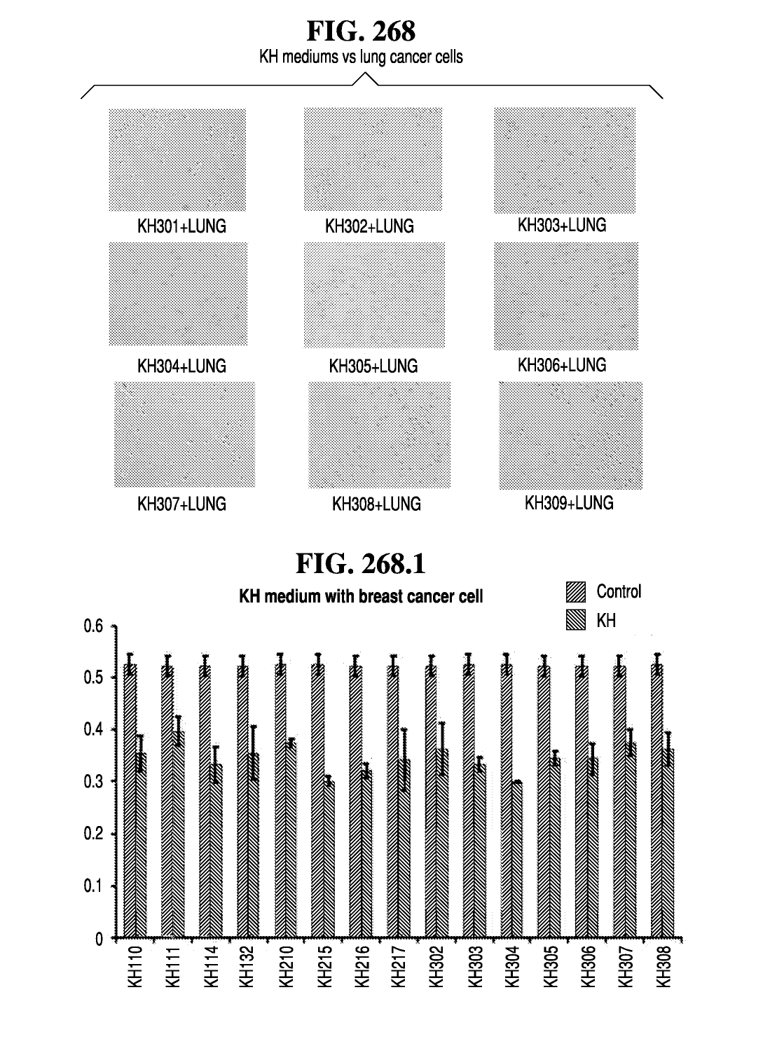

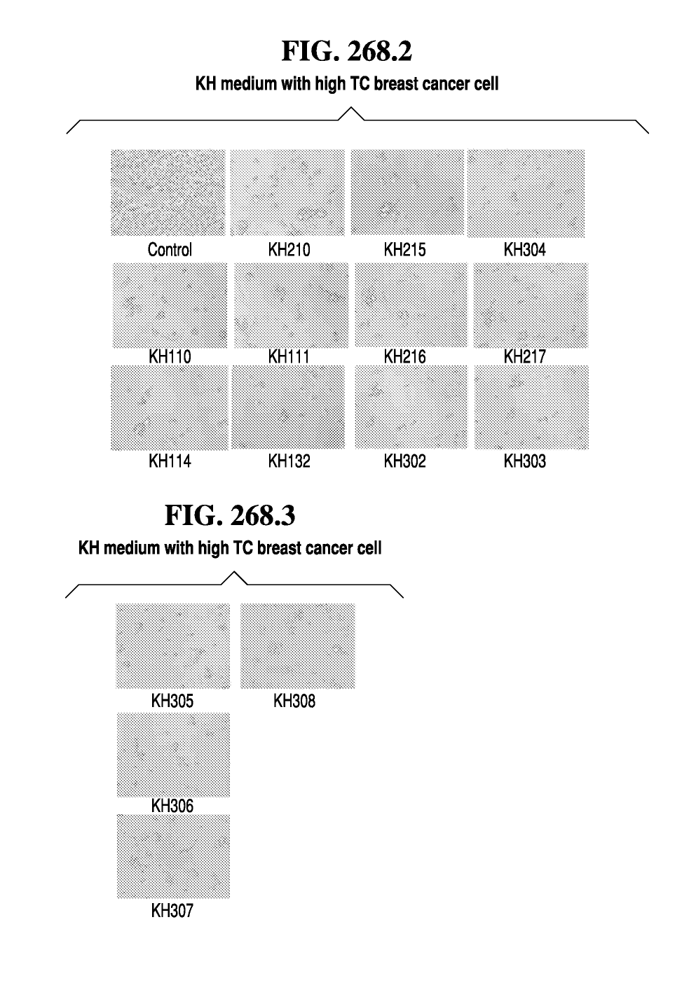

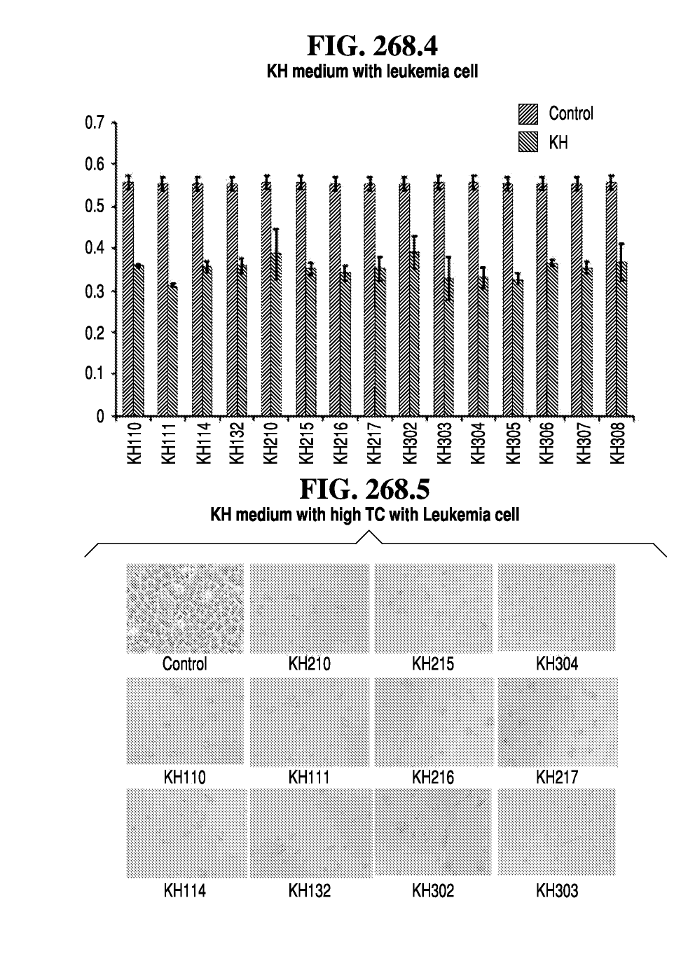

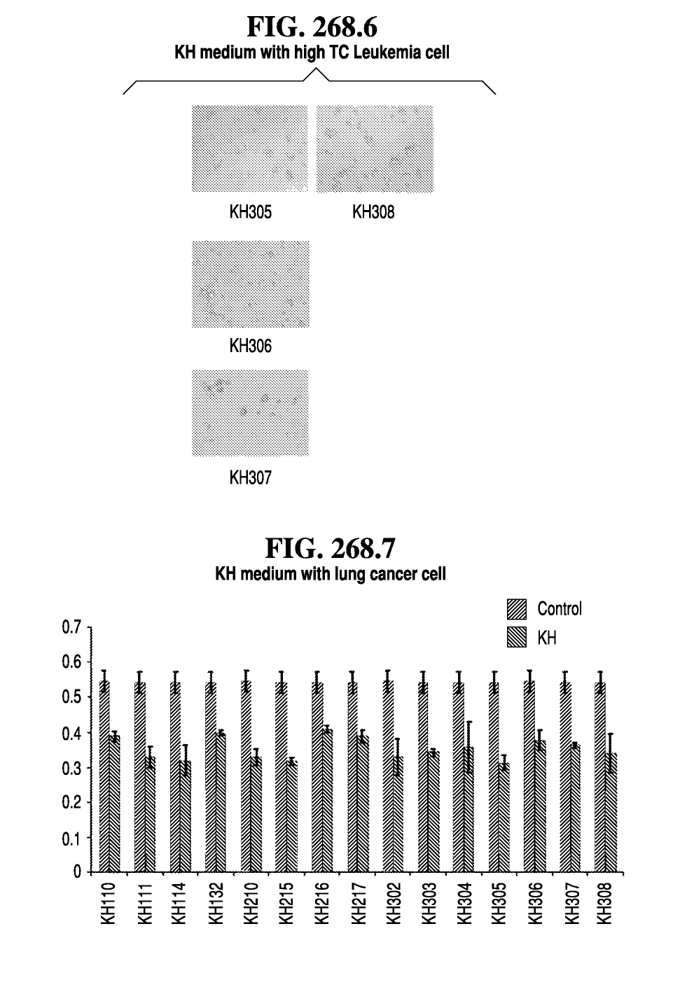

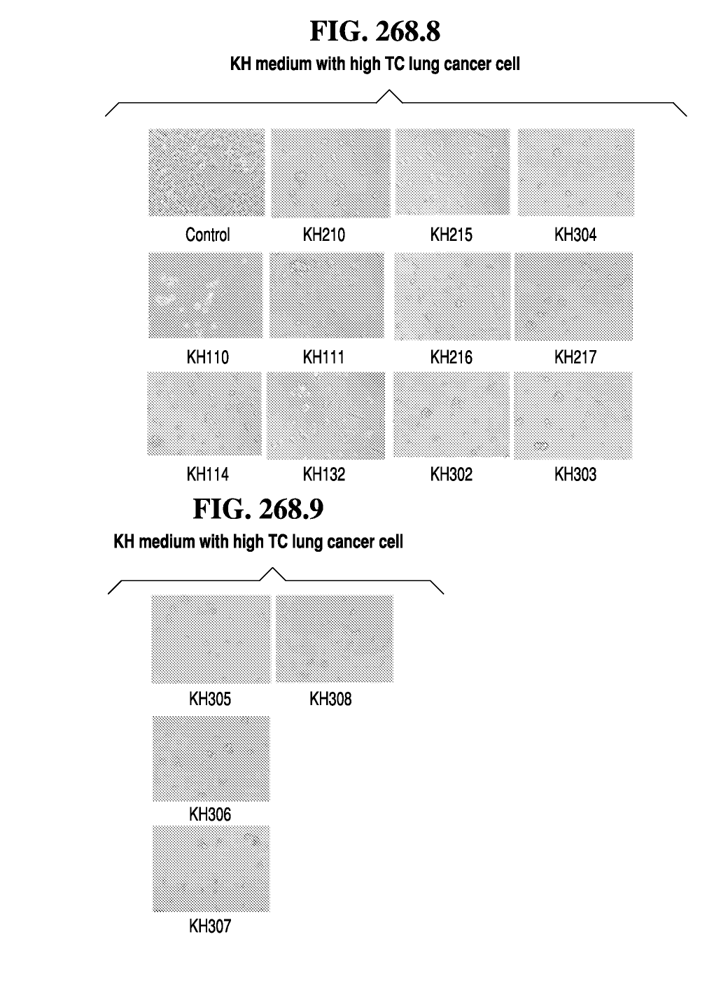

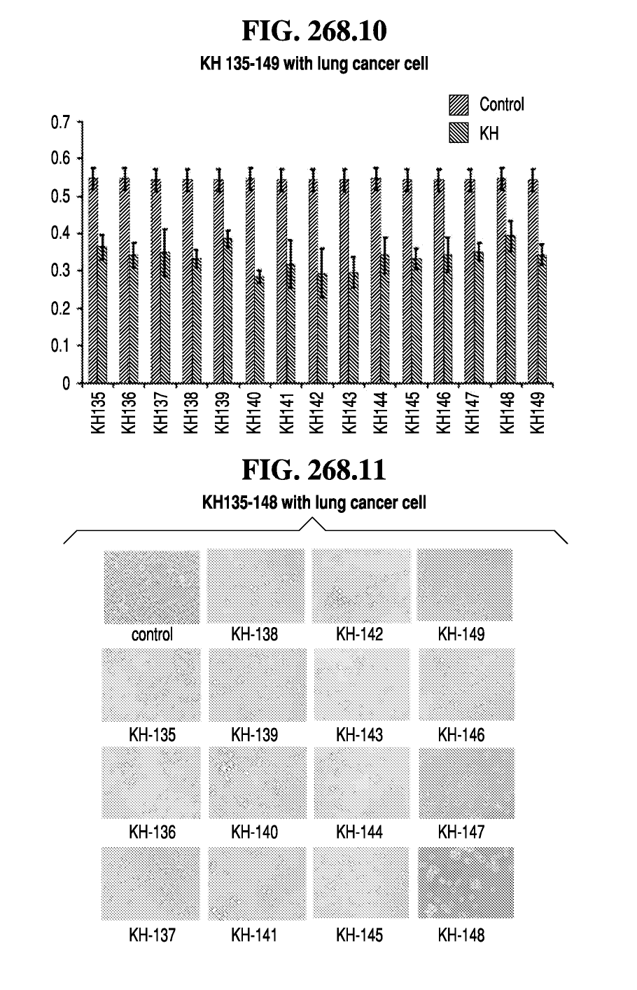

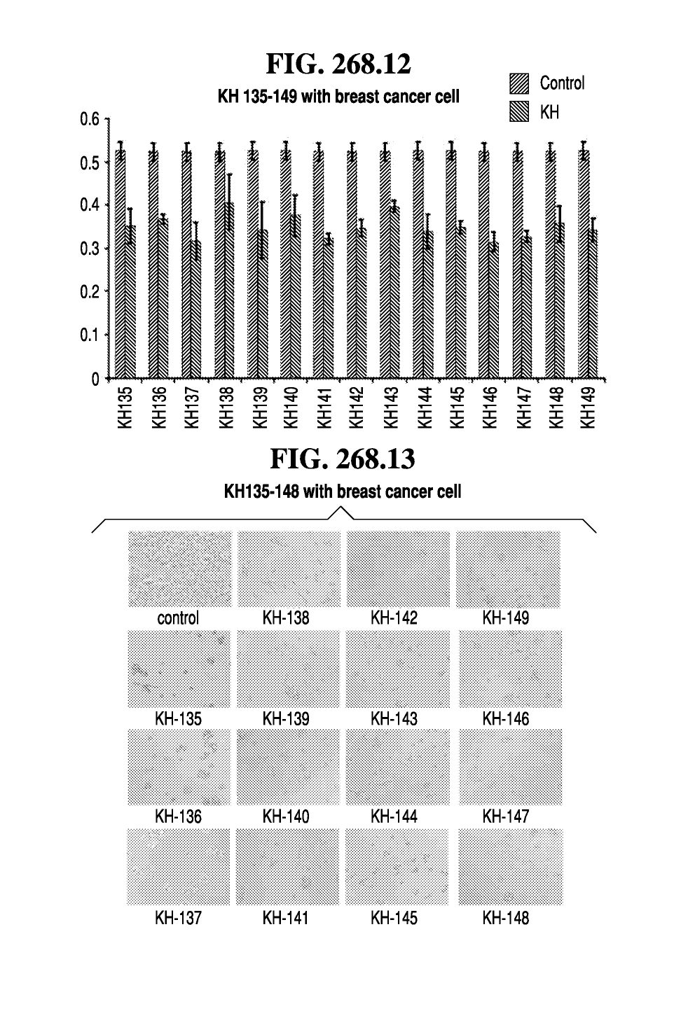

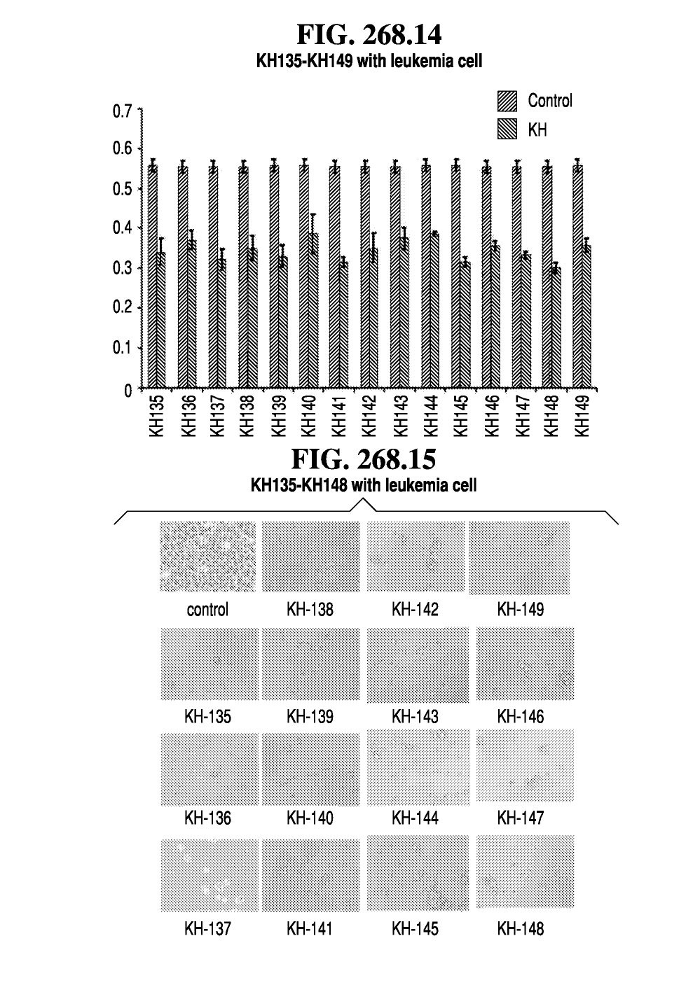

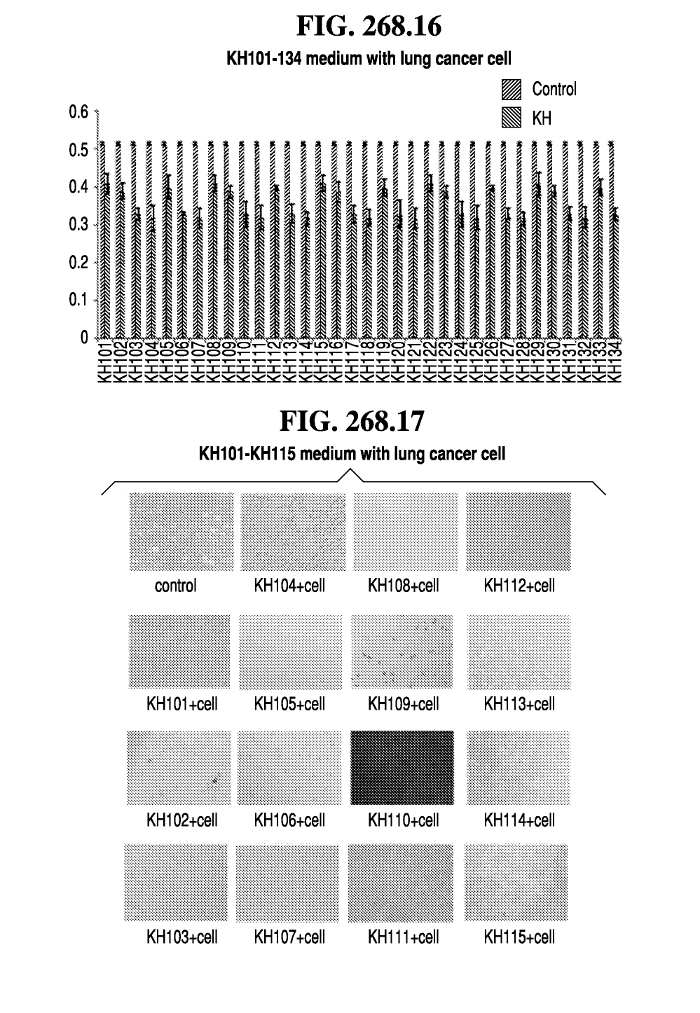

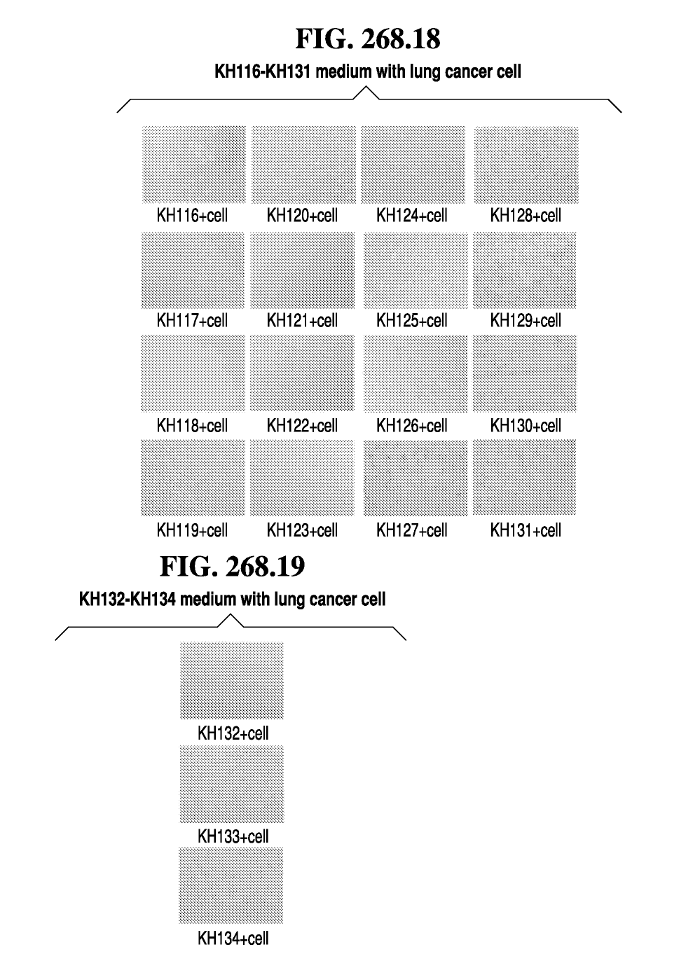

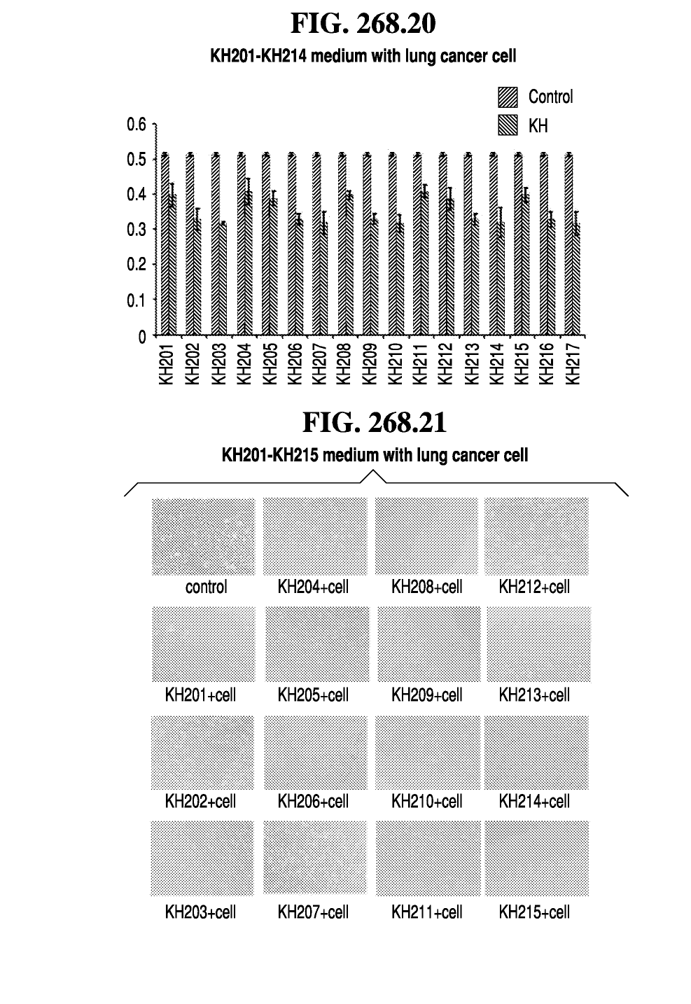

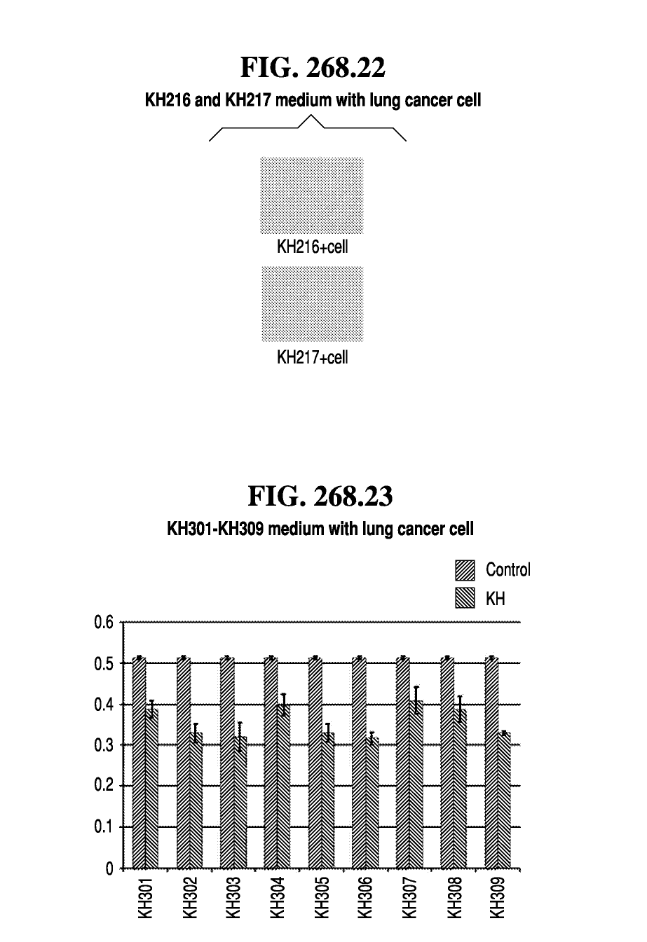

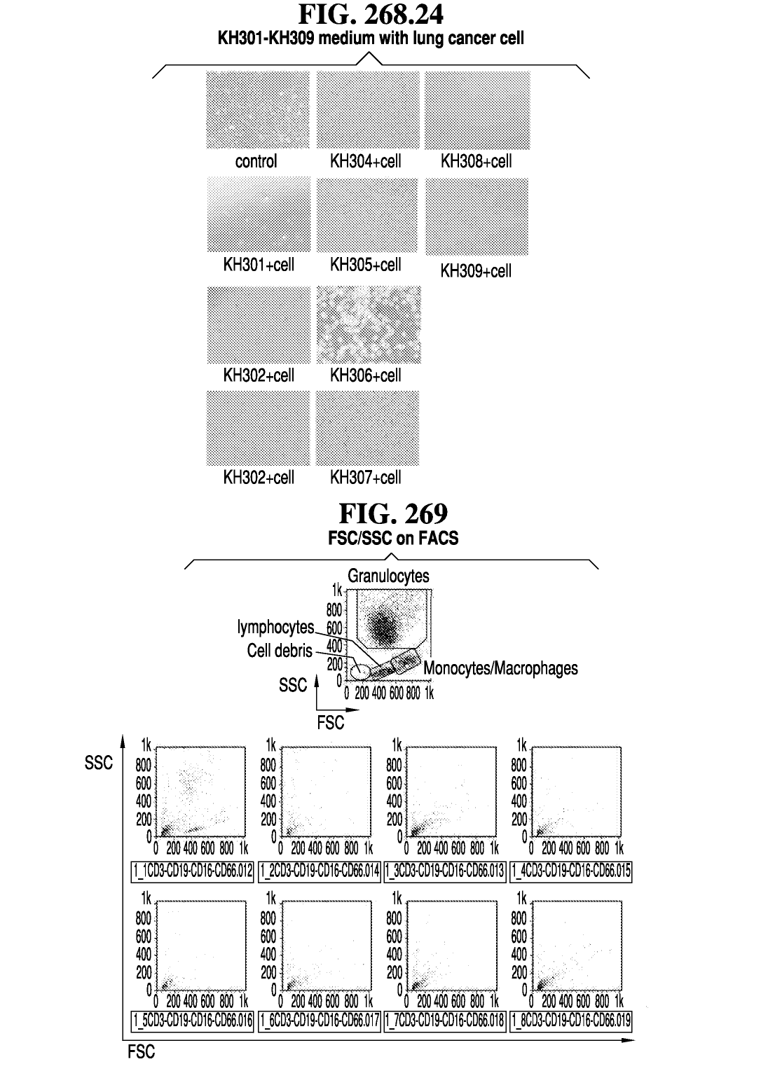

See FIG. 268.1, FIG. 268.2, FIG. 268.3, FIG. 268.4, FIG. 268.5, FIG. 268.6, FIG. 268.7, FIG. 268.8, FIG. 268.9, FIG. 268.10, FIG. 268.11, FIG. 268.12, FIG. 268.13, FIG. 268.14, FIG. 268.15, FIG. 268.16, FIG. 268.17, FIG. 268.18, FIG. 268.19, FIG. 268.20, FIG. 268.21, FIG. 268.22, FIG. 268.23, FIG. 268.24.

Another study has been performed to find out how the different mediums affect the CHO and HEK293 cells in comparison with the cell level indicators to find out which mediums have more effect than the others.

### Final Report

### Characterization of Cultured Cells for RAAS

### 1 Executive Summary

This study is to analyze the cells in culture by flow cytometric analysis. The samples were provided by the client. First, all the samples were counted individually with Vi-CELL Cell Viability Analyzer (Beckman Coulter) for cell number and viability. Then the samples were stained with cellular markers for different lineages including T cells, B cells, granulocytes, natural killer (NK) cells. Normal human peripheral blood sample was used as controls for the staining

Among 59 samples, 30 samples contained cells. Only 10 samples had total cell number above 1×10⁵ and only 5 samples reached viability above 90%. In comparison with forward scatter (FSC)/side scatter (SSC) of distinct subpopulations of human peripheral blood cells, such as lymphocytes, granulocytes, monocytes and macrophages, unknown samples didn't obtain the same distribution shown by FACS. Staining and distribution pattern of unknown samples also demonstrated they were not granulocytes, lymphocytes, or NK cells.

### 3 List of Abbreviations

| | |
|---|---|
| FACS | Flow Cytometry |
| BSA | Bovine serum albumin |
| FSC | Forward scatter |

| | |
|---|---|
| SSC | side scatter |
| NK cells | Natural killer cells |

### 4 Materials and Methods

### 4.1 Materials

### 4.1.1 Reagents

FITC, Anti-Human CD66, BD, Cat: 551479

FITC, Anti-Human CD34, BD, Cat: 560942

PE, Anti-Human CD3, BD, Cat: 561803

PE, Anti-Human CD146, BD, Cat: 561013

PE, Anti-Human CD56, BD, Cat: 561903

PE, Anti-Human CD14, BD, Cat: 561707

PE, Anti-Human CD11c, BD, Cat: 560999

PerCP-Cy5.5, Anti-Human CD16, BD, Cat: 560717

APC, Anti-Human CD19, BD, Cat: 561742

PE, Anti-Human CD41a, BD, Cat: 560979

ACK Lysis buffer, Invitrogen, Cat: A10492-01

PBS, Dycent Biotech (Shanghai) CO., Ltd. Cat: BJ141. FBS, Invitrogen Gibco, Cat: 10099141

BSA, Beyotime, ST023

### 4.1.2 Materials

Cell strainer (70 μm), BD, Cat: 352350

BD Falcon tubes (12×75 mm, 5 ml), BD, Cat: 352054

### 4.1.3 Equipments

Vi-CELL Cell Viability Analyzer, Beckman Coulter, Cat: 731050

FACSCalibur flow cytometer, BD, Cat: TY1218

### 4.2 Methods

### 4.2.1 Staining

Cells were placed into the 96-well (6×10⁵ cells/well) plate and blocked with 0.08% NaN3/PBS containing 1% FBS, 1% mouse serum and 2% BSA for 15 min at 4° C.
Cells were washed once with 1×PBS and resuspended with staining buffer (0.08% NaN3/PBS+1% FBS) with indicated antibodies for 30 min@ 4° C.
Cells were washed twice with 0.08% NaN3/PBS (200 μl per well) and resuspended with 400 μl 0.08% NaN3/PBS.
Excessive chunk from cell suspension were removed by filtrating through cell strainer. Cells were collected in BD Falcon tubes (12×75 mm, 5 ml) and analyzed by FACSCalibur.

### 5 Data Analysis

FACS data were analyzed by flowjo software.

### 6 Study Summary

6.1 Study initiation date and completion date

Cell samples were received on Apr. 26, 2012 and analyzed on April 27.

### 6.2 Study Purpose

The purpose of this study was to characterize the unknown cells.

### 6.3 Study Results

### 6.3.1 Cell Count

59 cell samples were counted individually using Vi-CELL Cell Viability Analyzer (Beckman Coulter). The detailed information was listed in Table 10.1.

TABLE 10.1
Cell counting

| Sample ID | Denisity × 10⁶/ml | Total cells | Viability(%) |
|---|---|---|---|
| 1_1 | 0.00E+00 | 0.00E+00 | |
| 1_2 | 0.00E+00 | 0.00E+00 | |
| 1_3 | 0.00E+00 | 0.00E+00 | |
| 1_4 | 0.00E+00 | 0.00E+00 | |
| 1_5 | 0.00E+00 | 0.00E+00 | |
| 1_6 | 0.00E+00 | 0.00E+00 | |
| 1_7 | 0.00E+00 | 0.00E+00 | |
| 1_8 | 0.00E+00 | 0.00E+00 | |
| 1_9 | 0.00E+00 | 0.00E+00 | |
| 1_10 | 0.00E+00 | 0.00E+00 | |
| 1_11 | 0.00E+00 | 0.00E+00 | |
| 1_12 | 0.00E+00 | 0.00E+00 | |
| 2_1 | 4.80E+04 | 4.80E+04 | |
| 66.7 | | | |
| 2_2 | 0.00E+00 | 0.00E+00 | |
| 2_3 | 0.00E+00 | 0.00E+00 | |
| 2_4 | 0.00E+00 | 0.00E+00 | |
| 2_5 | 0.00E+00 | 0.00E+00 | |
| 2_6 | 0.00E+00 | 0.00E+00 | |
| 2_7 | 0.00E+00 | 0.00E+00 | |
| 2_8 | 0.00E+00 | 0.00E+00 | |
| 2_9 | 0.00E+00 | 0.00E+00 | |
| 2_10 | 0.00E+00 | 0.00E+00 | |
| 2_11 | 0.00E+00 | 0.00E+00 | |
| 2_12 | 0.00E+00 | 0.00E+00 | |
| 3_1 | 4.80E+04 | 4.80E+04 | |
| 57.1 | | | |
| 3_2 | 2.40E+04 | 2.40E+04 | |
| 25 | | | |
| 3_3 | 5.90E+04 | 5.90E+04 | |
| 41.7 | 0.00E+00 | 0.00E+00 | |
| 3_4 | | | |
| 3_5 | 2.40E+04 | 2.40E+04 | |
| | 0.00E+00 | 0.00E+00 | |
| 3_6 | 3.60E+04 | 3.60E+04 | 50 |
| 3_7 | 2.40E+04 | 2.40E+04 | 20 |
| 3_8 | 2.40E+04 | 2.40E+04 | 40 |
| 3_9 | 3.60E+04 | 3.60E+04 | 100 |
| 3_10 | 3.60E+04 | 3.60E+04 | 60 |
| 3_11 | 9.50E+04 | 9.50E+04 | 57.1 |
| 3_12 | 2.40E+04 | 2.40E+04 | 40 |
| 4_1 | 9.50E+04 | 9.50E+04 | 32 |
| 4_2 | 3.80E+05 | 3.80E+05 | 69.6 |
| 4_3 | 3.30E+05 | 3.30E+05 | 93.3 |
| 4_4 | 1.20E+05 | 1.20E+05 | 35.7 |
| 4_5 | 3.70E+05 | 3.70E+05 | 72.1 |
| 4_6 | 2.50E+05 | 2.50E+05 | 87.5 |
| 4_7 | 1.80E+05 | 1.80E+05 | 37.5 |
| 4_8 | 2.40E+05 | 2.40E+05 | 44.4 |
| 4_9 | 3.30E+05 | 3.30E+05 | 96.6 |
| 5_1 | 1.80E+05 | 1.80E+05 | 48.4 |
| 5_2 | 2.40E+05 | 2.40E+05 | 55.6 |
| 5_3 | 3.00E+05 | 3.00E+05 | 92.6 |
| 5_4 | 2.70E+05 | 2.70E+05 | 79.3 |
| 5_5 | 2.10E+05 | 2.10E+05 | 51.4 |
| 5_6 | 2.40E+04 | 2.40E+04 | 66.7 |
| 6_1 | 1.20E+04 | 1.20E+04 | 50 |
| 6_2 | 1.20E+04 | 1.20E+04 | 50 |
| 6_3 | 1.20E+04 | 1.20E+04 | 100 |
| 6_4 | 0.00E+00 | 0.00E+00 | |
| 6_5 | 0.00E+00 | 0.00E+00 | |
| 6_6 | 0.00E+00 | 0.00E+00 | |
| 6_7 | 0.00E+00 | 0.00E+00 | |
| | 28.6 | 6_8 | |

Among 59 samples, 30 samples had countable cells. 10 samples highlighted in yellow had total cell number above 1×10⁵. Only 5 samples reached viability above 90%.

### 6.3.2 FSC/SSC Analysis by FACS

Among 59 samples, all the samples showed lots of cell debris by FSC/SSC. None of the samples were found to have the same distribution pattern as granulocytes, lymphocytes, monocytes and macrophages, suggesting that there were no visible granulocytes, lymphocytes, monocytes or macrophages in the tested samples (FIG. 1 to FIG. 9).

FIG. 269. FSC/SSC on FACS

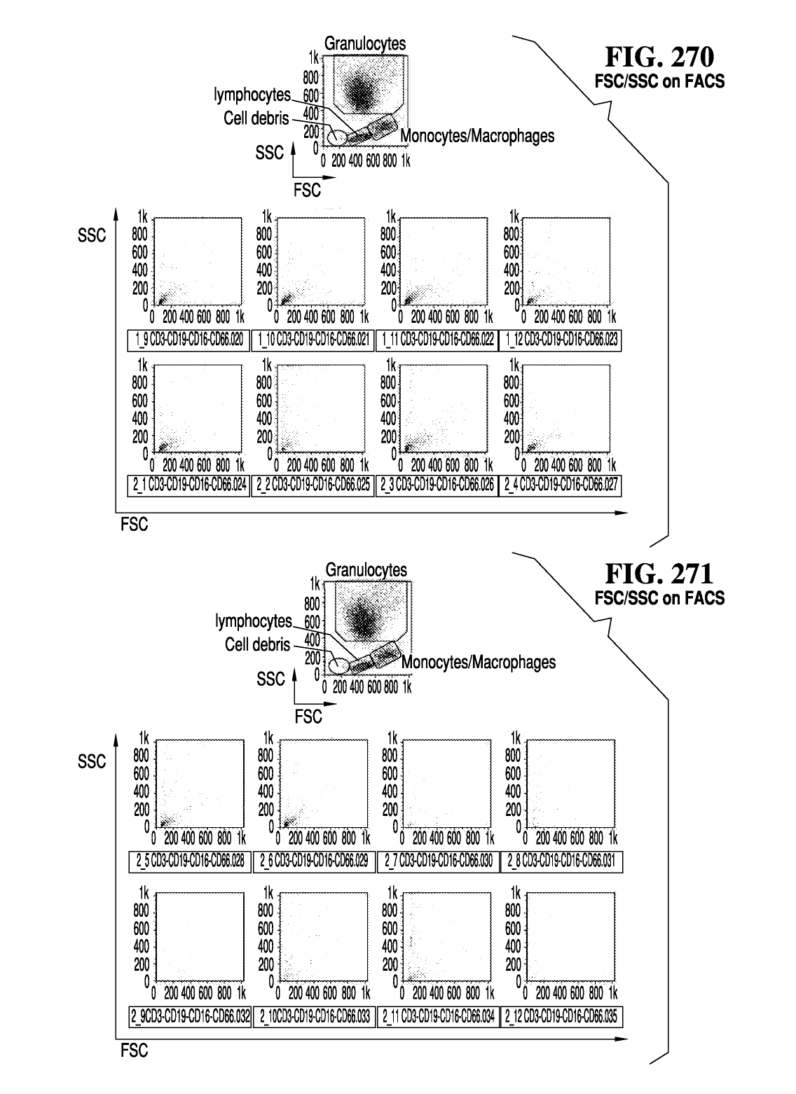

FIG. 270. FSC/SSC on FACS

FIG. 271. FSC/SSC on FACS

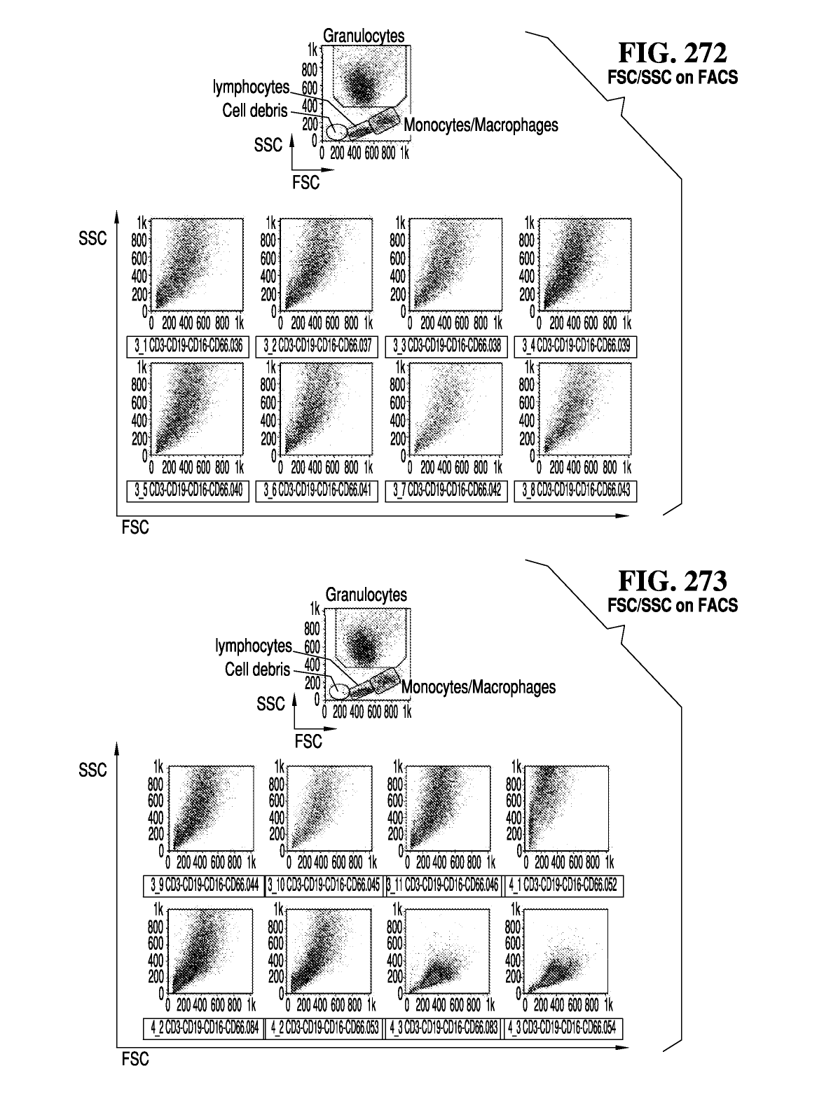

FIG. 272. FSC/SSC on FACS

FIG. 273. FSC/SSC on FACS

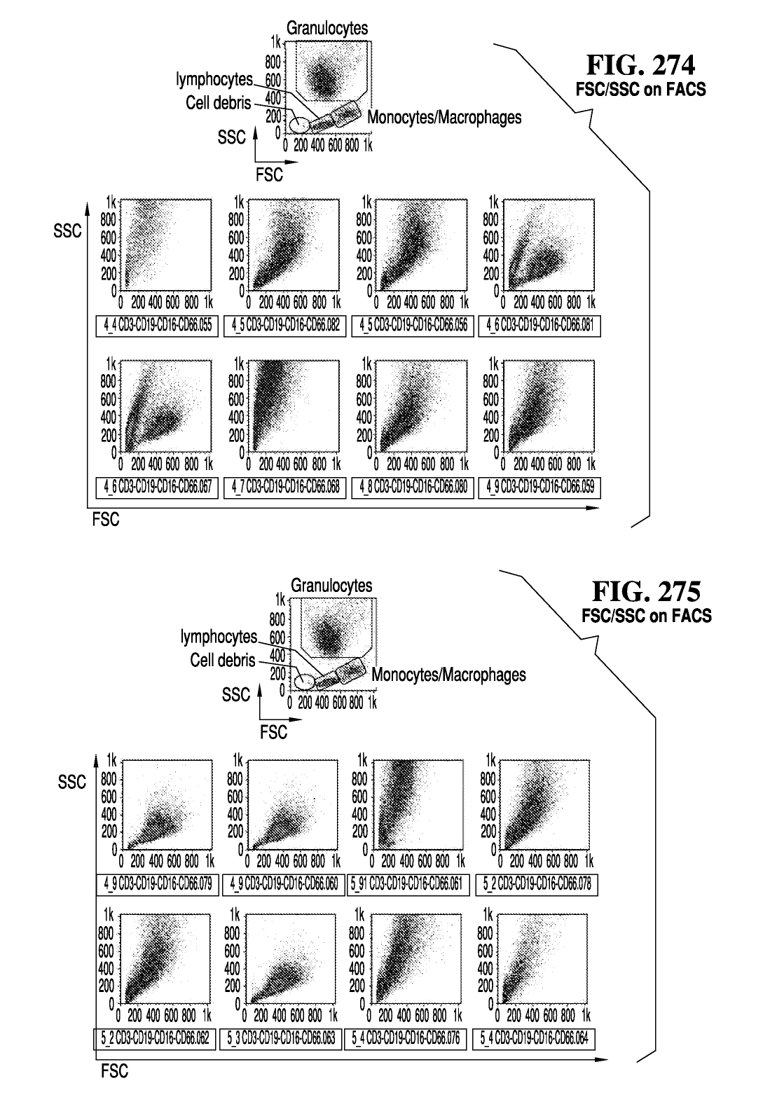

FIG. 274. FSC/SSC on FACS

FIG. 275. FSC/SSC on FACS

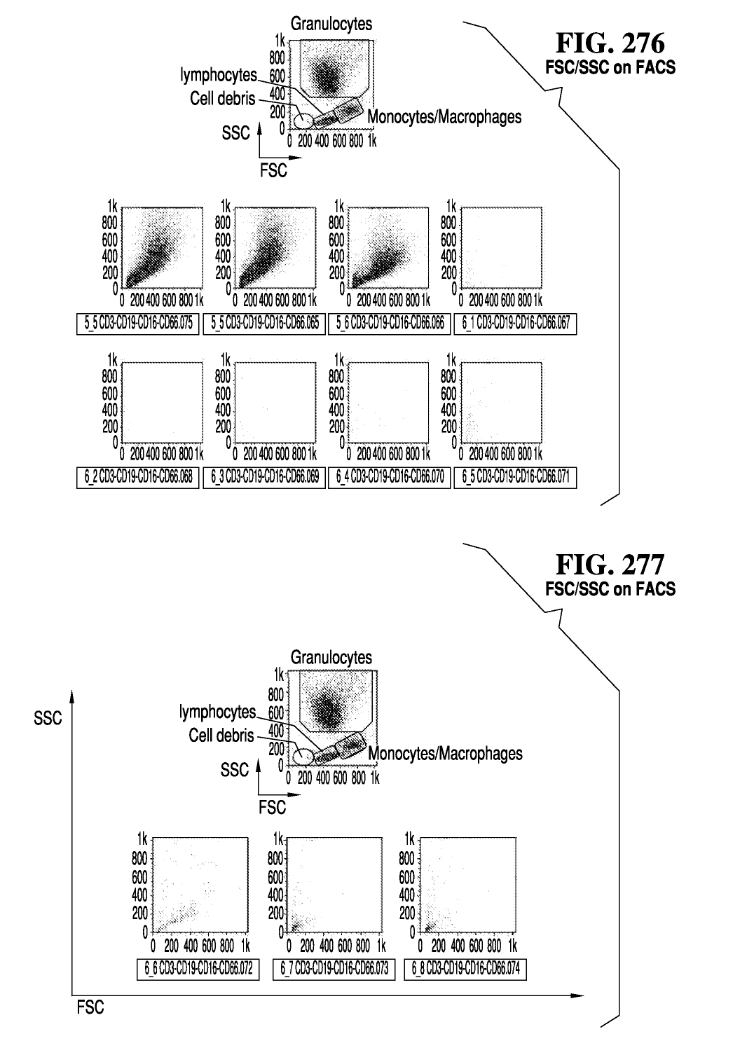

FIG. 276. FSC/SSC on FACS

FIG. 277. FSC/SSC on FACS

6.3.3 Comparison with Human T/B Cells by FACS

Human peripheral blood and test samples were stained side by side with the same antibodies. B and T cell populations were identified by FACS (FIG. 10 to FIG. 16). The data did not show a convincing population of T or B cells.

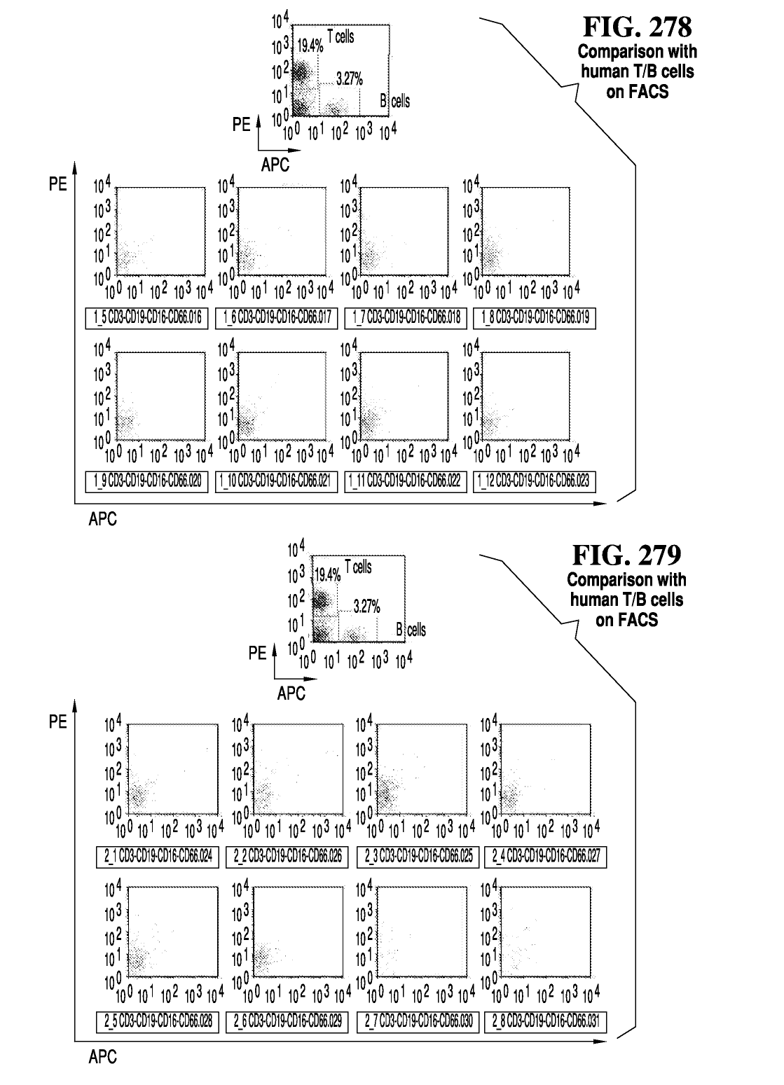

FIG. 278. Comparison with human T/B cells on FACS

FIG. 279. Comparison with human T/B cells on FACS

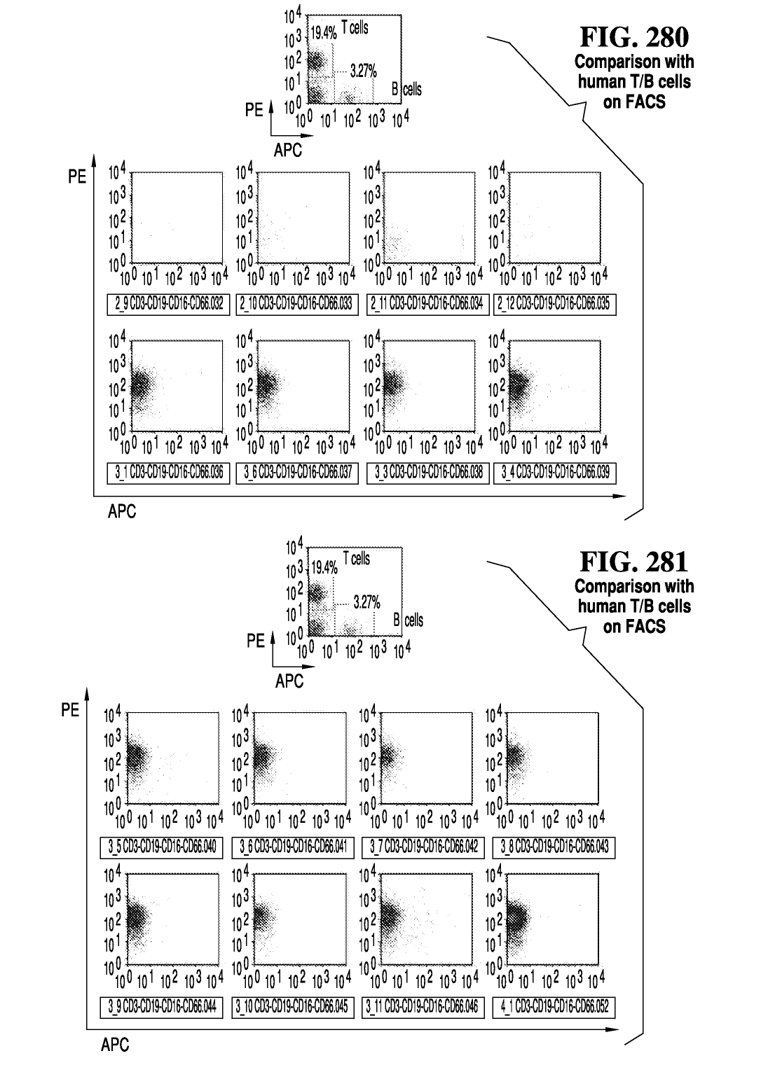

FIG. 280. Comparison with human T/B cells on FACS

FIG. 281. Comparison with human T/B cells on FACS

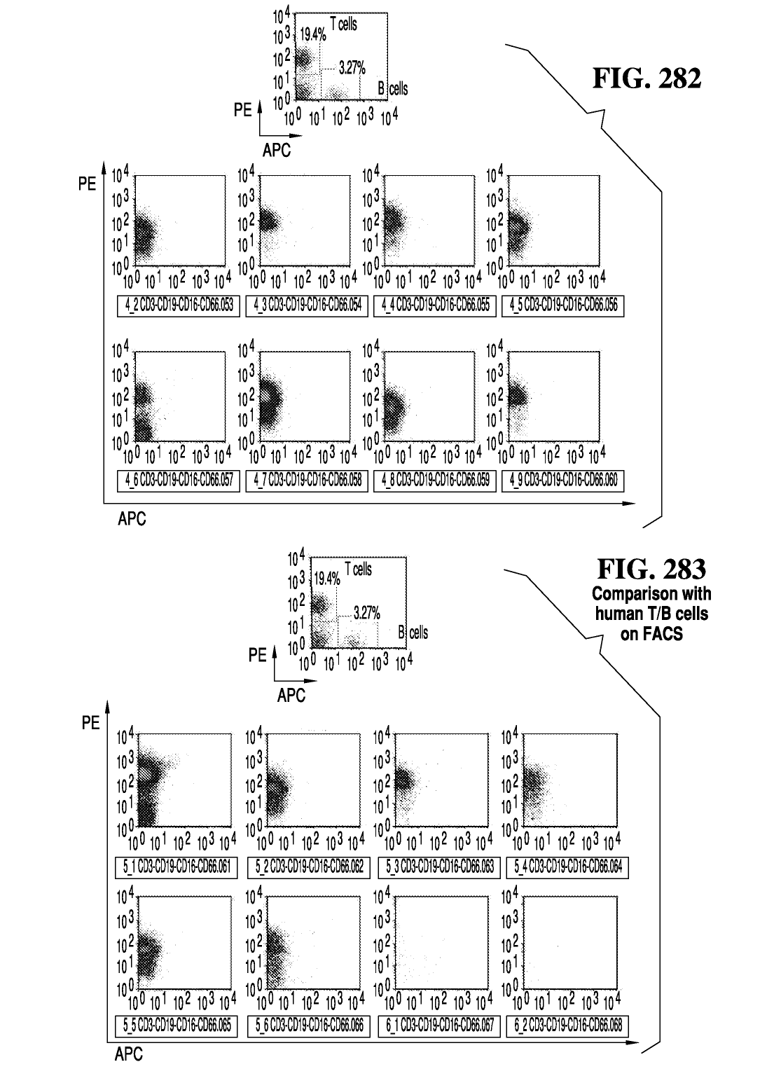

FIG. 282. Comparison with human T/B cells on FACS

FIG. 283. Comparison with human T/B cells on FACS

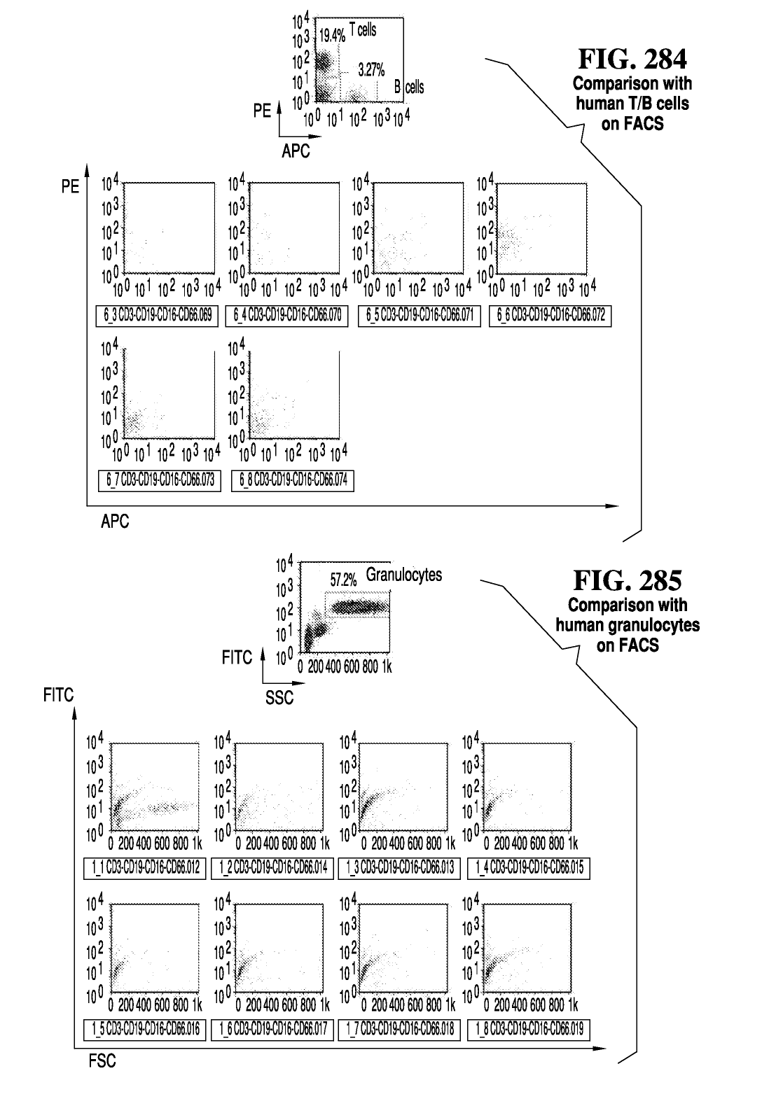

FIG. 284. Comparison with human T/B cells on FACS

6.3.4 Comparison Unknown Samples with Granulocytes by FACS

In addition to staining of T and B lymphocytes, human peripheral blood and test samples were stained simultaneously with the same antibodies and granulocytes were further identified by FACS. No granulocytes were found in all the test samples (FIG. 17 to FIG. 24).

FIG. 285—Comparison with human granulocytes on FACS

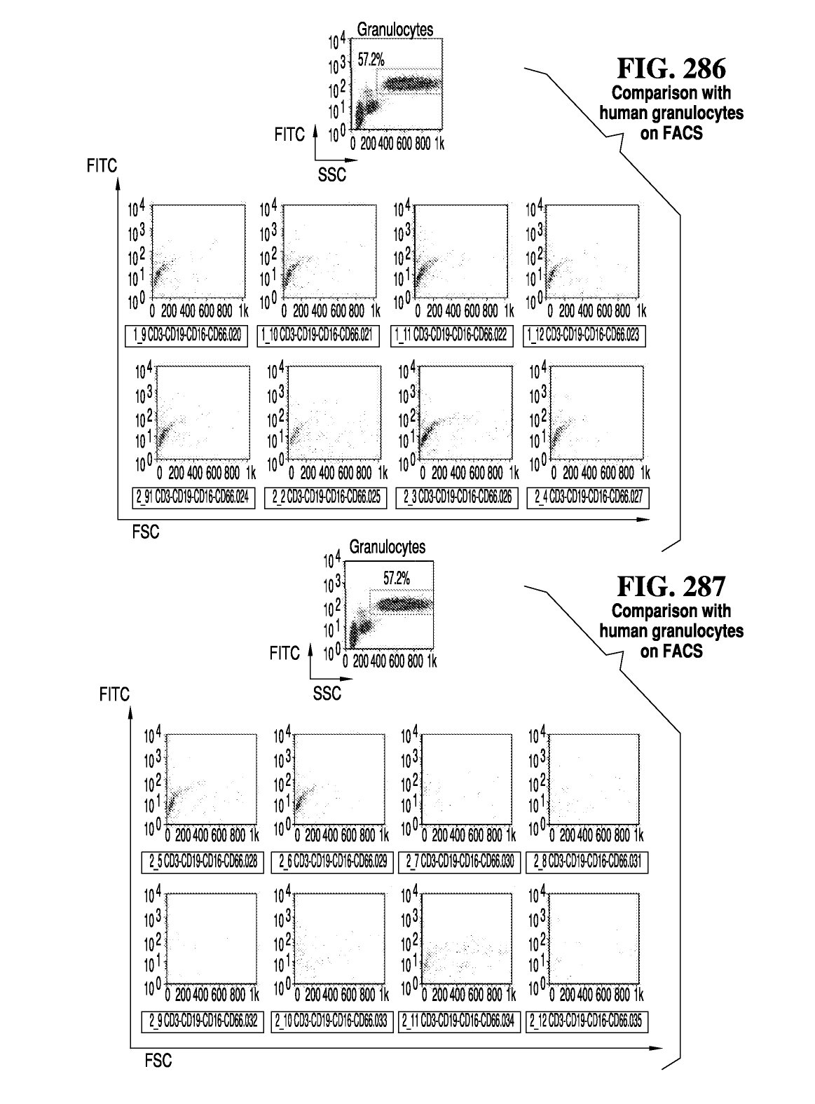

FIG. 286—Comparison with human granulocytes on FACS

FIG. 287—Comparison with human granulocytes on FACS

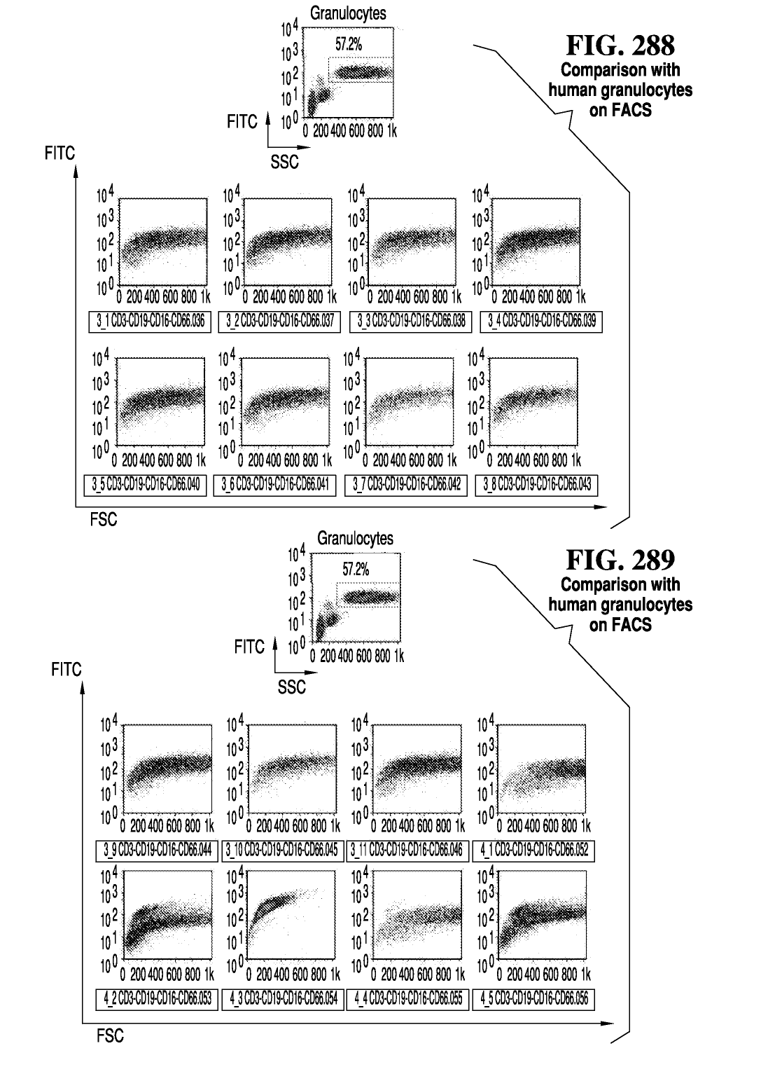

FIG. 288—Comparison with human granulocytes on FACS

FIG. 289—Comparison with human granulocytes on FACS

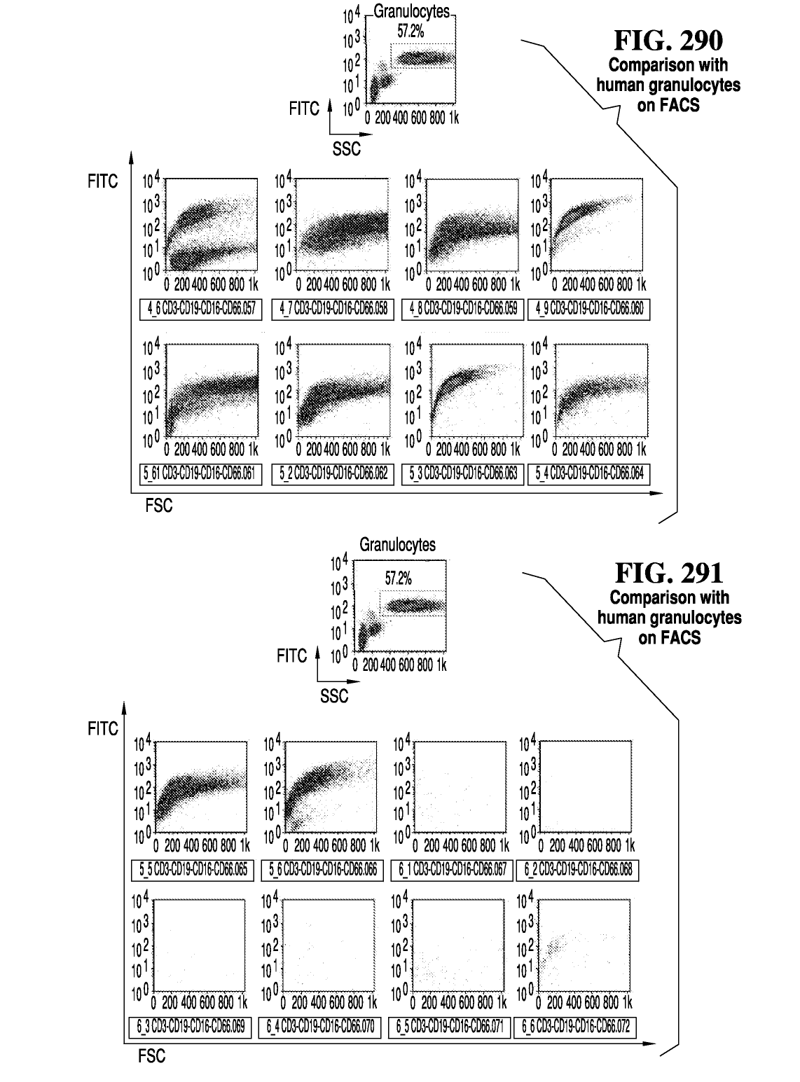

FIG. 290—Comparison with human granulocytes on FACS

FIG. 291—Comparison with human granulocytes on FACS

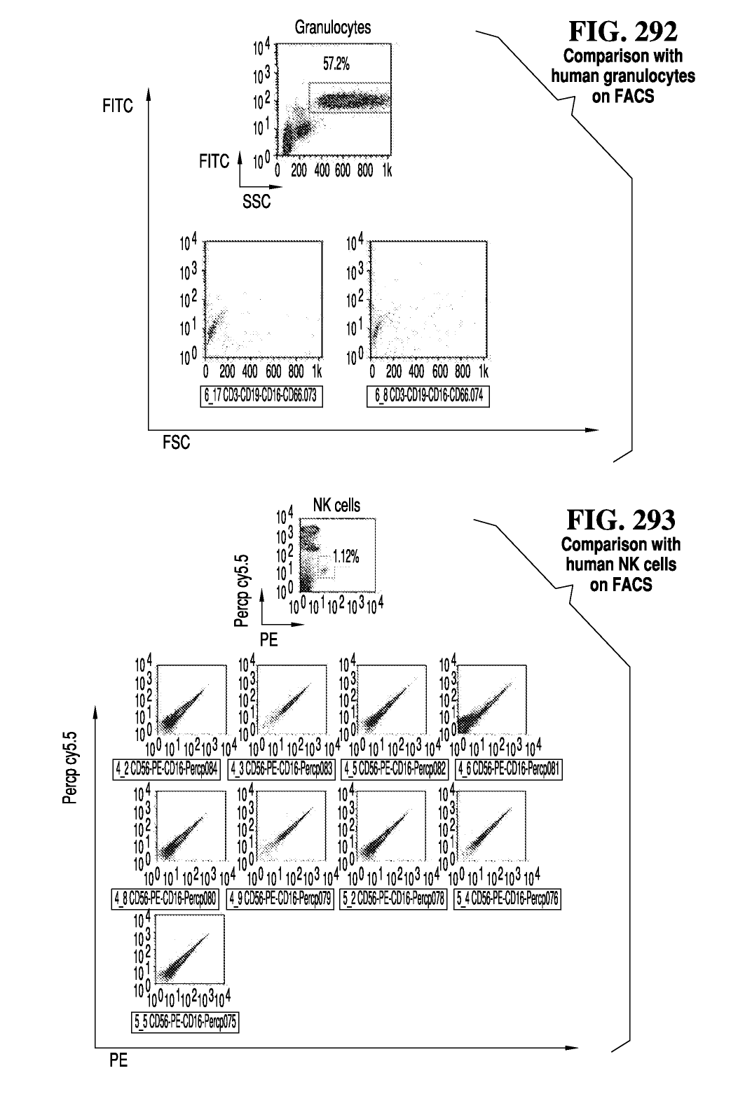

FIG. 292—Comparison with human granulocytes on FACS

6.3.5 Comparison Unknown Samples with NK Cells by FACS

None of the samples were found to contain NK cells (FIG. 25).

FIG. 293—Comparison with human NK cells on FACS

### 7. Conclusion

The characterization of unknown samples was carried out by staining with different cell surface markers for distinct cell lineages. Normal human peripheral blood cells were used as controls.

Vi-CELL cell viability analysis showed that 30 samples out of 59 samples had cells. Among these, only 10 samples had total cell number above 1×10⁵ and only 5 samples reached viability above 90% (Table 10.1).

FACS analysis indicated that the test samples may not contain any of the typical cells present in human peripheral blood.

### Quantification of Cholesterol and Triglyceride Levels in RAAS Products

### I. General Information

### 1.1 Experimental Requested by:

Mr. Kieu Hoang from Shanghai RAAS

### 1.2 Project ID/Code:

RAAS/T01

### 1.3 Experimental Objective:

Lipids panel tests for RAAS products (TC, TG, HDL and LDL) by Biovision kits

### 1.4 Experiment Number:

LIPIDS2k11-01

### 1.5 Target Start Date:

Sep. 12, 2011

### II. Introduction

The objective of this study was to quantify total cholesterol (TC), high-density-lipoprotein (HDL) cholesterol, low-density-lipoprotein (LDL) cholesterol and Triglyceride (TG) levels of RAAS products.

Cholesterol plays a central role in various disease developments. It is well known that low levels of HDL and high level of LDL are associated with an increased risk of cardiovascular events. BioVision's HDL and LDL/VLDL Cholesterol Quantification Kits provide a simple quantification method of HDL and LDL/VLDL after a convenient separation of HDL from LDL and VLDL (very low-density lipoprotein) in serum samples. In the assay, cholesterol oxidase specifically recognizes free cholesterol and produces products which react with probe to generate color (λ=570 nm) and fluorescence (Ex/Em=538/587 nm). Cholesterol esterase hydrolizes cholesteryl ester into free cholesterol, therefore, cholesterol ester and free cholesterol can be detected separately in the presence and absence of cholesterol esterase in the reactions.

The Cholesterol/Cholesteryl Ester Quantitation Kit provides a simple method for sensitive quantification of free cholesterol, cholesteryl esters, or both by colorimetric or fluorometric methods. Majority of the cholesterol in blood is in the form of cholesteryl esters which can be hydrolyzed to cholesterol by cholesterol esterase. Cholesterol is then oxidized by cholesterol oxidase to yield H₂O₂ which reacts with a sensitive cholesterol probe to produce color (λmax=570 nm) and fluorescence (Ex/Em=535/587 nm). The assay detects total cholesterol (cholesterol and cholesteryl esters) in the presence of cholesterol esterase or free cholesterol in the absence of cholesterol esterase in the reaction. Cholesteryl ester can be determined by subtracting the value of free cholesterol from the total (cholesterol plus cholesteryl esters).

Triglycerides are the main constituent of vegetable oil, animal fat, LDL and VLDL, and play an important role as transporters of fatty acids as well as serving as an energy source. Triglycerides are broken down into fatty acids and glycerol, after which both can serve as substrates for energy producing and metabolic pathways. High blood levels of triglycerides are implicated in atherosclerosis, heart disease and stroke as well as in pancreatitis. The Triglyceride Quantification Kit provides a sensitive, easy assay to measure triglyceride concentration in variety of samples. In the assay, triglycerides are converted to free fatty acids and glycerol. The glycerol is then oxidized to generate a product which reacts with the probe to generate colorimetric (spectrophotometry at λ=570 nm) and fluorometric (Ex/Em=535/587 nm) methods. The kit can detect 1 pmol-10 nmol (or 1˜10000 μM range) of triglyceride in various samples.

### III. Sample Lists:

| | Samples | | Sample | Samples |
|---|---|---|---|---|
| | received | Volume | preparation | to test |
| 1. AFOD | 160 bottles | 2 ml/ | use as | 3 bottles |
| | | bottle | supplied | |
| 2. AFOD RAAS 1 | 6 bottles | 50 ml/ | use as | 3 bottles |
| | | bottle | supplied | |
| 3. AFOD RAAS 2 | 6 bottles | 50 ml/ | use as | 3 bottles |
| | | bottle | supplied | |
| 4. AFCC RAAS 1 | 5 bottles | powder | dilute in | 3 bottles |
| | | | 10 ml H₂0 | |
| 5. AFCC RAAS 2 | 5 bottles | powder | dilute in | 3 bottles |
| | | | 10 ml H₂0 | |
| 6. AFCC RAAS 3 | 4 bottles | powder | dilute in | 3 bottles |
| | | | 2 ml H₂0 | |
| 7. AFCC RAAS 4 | 5 bottles | powder | dilute in | 3 bottles |
| | | | 10 ml H₂0 | |
| 8. AFCC RAAS 5 | 5 bottles | powder | dilute in | 3 bottles |
| | | | 2 ml H₂0 | |
| 9. AFOD RAAS 3 | 2 bottles | 2 ml/ | use as | 2 bottles |
| | | bottle | supplied | |
| 12. RE-VIII RAAS | 1 kit | powder | dilute in | 1 bottles |
| | | | diluents | |

### IV. Methods:

### 4A. Total Cholesterol/Cholesteryl Ester Quantification by Fluorometric Method (TC)

Cholesterol/Cholesteryl Ester Quantitation Kit (Catalog #K603-100; 100 assays; Store at −20° C.)

### 1. Kit Contents:

| Components | K622-100 | | Cap Code | Part Number |
|---|---|---|---|---|
| Cholesterol Assay Buffer | 25 | ml | WM | K603-100-1 |
| Cholesterol Probe (in DMSO, | 200 | μl | Red | K603-100-2A |
| anhydrous) | | | | |
| Enzyme Mix (lyophilized) | 1 | vial | Green | K603-100-4 |
| Cholesterol Esterase | 1 | vial | Blue | K603-100-5 |
| (lyophilized) | | | | |
| Cholesterol Standard | 100 | μl | Yellow | K603-100-6 |
| (2 μg/μl) | | | | |

### 2. Storage and Handling:

Store kit at −20° C., protect from light. Warm to room temperature before use. Keep enzymes and cholesterol standard on ice while using.

### 3. Reagents Preparation:

Cholesterol Probe: Warm to room temperature to thaw the DMSO solution before use. Store at −20° C., protect from light.

Cholesterol Esterase: Dissolve in 220 μl Cholesterol Assay Buffer before use. Aliquot and store at −20° C.

Enzyme Mix: Dissolve in 220 μl Cholesterol Assay Buffer before use. Aliquot and store at −20° C.

### 4. Cholesterol Assay Protocol:

4.1. Standard Curve Preparation:

Dilute the Cholesterol Standard to 25 ng/μl by adding 10 μl of the Cholesterol Standard to 790 μl of Cholesterol Assay Buffer, mix well. Add 0, 4, 8, 12, 16, 20 μl into a series of wells.

Adjust volume to 50 μl/well with Cholesterol Assay Buffer to generate 0, 0.1, 0.2, 0.3, 0.4, 0.5 μg/well of the Cholesterol Standard.

4.2. Sample Preparation: Add 5 μl test samples in a 96-well clear bottom black plate, Adjust to the final volume of 50 μl/well with Triglyceride Assay Buffer.

4.3. Cholesterol Reaction Mix: Mix enough reagents for the number of samples and standards to be performed: For each well, prepare a total 50 μl Reaction Mix:

45.6 μl Cholesterol Assay Buffer

0.4 μl Cholesterol Probe

2 μl Cholesterol Enzyme Mix

2 μl Cholesterol Esterase

4.4. Mix well Add 50 μl of the Reaction Mix to each well containing standard or test samples.

4.5. Incubate the reaction for 60 minutes at 37° C., protect from light.

4.6. Measure fluorescence at Ex/Em 535/590 nm in ENSPIRE

4.7. Calculations: Subtract 0 standard reading from readings. Plot the standard curve. Apply the sample readings to the standard curve to determine sample cholesterol amount in the reaction well.

Sample cholesterol concentrations: C=A/V (μg/μl)

Where: A is the sample cholesterol amount from the standard curve (μg). V is original sample volume added to the sample reaction well (μl).

### 4B. HDL and LDL/VLDL Cholesterol Quantification by Fluorometric Method (HDLC and LDLC/VLDLC)

HDL and LDL&VLDL Cholesterol Quantification Kit (Catalog #K613-100; 100 assays; Store at −20° C.)

### 1. Kit Contents:

| Components | Volume | | Cap Code | Part No. |
|---|---|---|---|---|
| Cholesterol Assay Buffer | 25 | ml | WM | K613-100-1 |
| 2× LDL/VLDL Precipitation | 10 | ml | NM | K613-100-2 |
| Buffer | | | | |
| Cholesterol Probe (in DMSO, | 200 | μl | Red | K613-100-3A |
| anhydrous) | | | | |
| Enzyme Mix (Lyophilized) | 1 | vial | Green | K613-100-5 |
| Cholesterol Esterase | 1 | vial | Blue | K613-100-6 |
| (Lyophilized) | | | | |
| Cholesterol Standard (2 μg/μl) | 100 | μl | Yellow | K613-100-7 |

### 2. Reagent Preparation:

Cholesterol Probe: Warm to room temperature, store at −20° C., protect from light. Cholesterol

Esterase: Dissolve in 220 μl Cholesterol Assay Buffer. Aliquot and store at −20° C.

Enzyme Mix: Dissolve in 220 μl Cholesterol Assay Buffer prior to use. Aliquot and store at −20° C.

### 3. HDL and LDL/VLDL Cholesterol Assay Protocol:

3.1. Separation of HDL and LDL/VLDL: Mix 100 μl of 2× Precipitation Buffer with 100 μl of serum sample in microcentrifuge tubes. Incubate 10 min at RT, centrifuge at 2000×g (5000 rpm) for 10 min. Transfer the supernatant (HDL) into new labeled tubes. Spin the precipitates (LDL/VLDL) again, Remove HDL supernatant. Resuspend the precipitate in 200 μl PBS.

Note A: If the supernatant is cloudy, the sample should be re-centrifuged. If the sample remains cloudy, dilute the sample 1:1 with PBS, and repeat the separation procedure. Multiply final results by two (2) due to the dilution with the 2× Precipitation Buffer.

3.2. Standard Curve and Sample Preparations: Dilute the Cholesterol Standard to 25 ng/μl by adding 10 μl of the Cholesterol Standard to 790 μl of Cholesterol Assay Buffer, Add 0, 4, 8, 12, 16, 20 μl into a series of wells in a 96-well clear bottom black plate. Adjust volume to 50 μl/well with Cholesterol Assay Buffer to generate 0, 0.1, 0.2, 0.3, 0.4, 0.5 μg/well of the Cholesterol Standard. Use 5 μl of the HDL or LDL/VLDL fraction, adjust the total volume to 50 μl/well with Cholesterol Assay Buffer.

3.3. Reaction Mix Preparations: Mix enough reagents for the number of assays performed. For each assay, prepare a total 50 μl Reaction Mix containing:

45.6 μl Cholesterol Assay Buffer

0.4 μl Cholesterol Probe

2 μl Enzyme Mix

2 μl Cholesterol Esterase

3.4. Add 50 μl of the Reaction Mix to each well containing the Cholesterol Standard or test samples, mix well.

3.5. Incubate the reaction for 60 minutes at 37° C., protect from light. Measure fluorescence at Ex/Em 538/587 nm in ENSPIRE

3.6. Calculations: Subtract 0 standard reading from readings. Plot the standard curve. Apply the sample readings to the standard curve to determine sample cholesterol amount in the reaction well.

Sample cholesterol concentrations:C=A/V(μg/μl)

Where: A is the sample cholesterol amount from the standard curve (μg). V is original sample volume added to the sample reaction well (μl).

### 4C Triglyceride Quantification by Fluorometric Method (TG)

Triglyceride Quantification Kit (Catalog #K622-100; 100 assays; Store at −20° C.)

### 1. Kit Contents:

| Components | K622-100 | | Cap Code | Part Number |
|---|---|---|---|---|
| Triglyceride Assay Buffer | 25 | ml | WM | K622-100-1 |
| Triglyceride Probe (lyophilized) | 1 | vial | Red | K622-100-2 |
| Dimethylsulfoxide (DMSO, | 0.4 | ml | Brown | K622-100-3 |
| Anhydrous) | | | | |
| Lipase | 0.5 | ml | Blue | K622-100-4 |
| Triglyceride Enzyme Mix | 1 | vial | Green | K622-100-5 |
| (lyophilized) | | | | |
| Triglyceride Standard (1 mM) | 0.2 | ml | Yellow | K622-100-6 |

### 2. Storage and Handling:

Store kit at −20° C., protect from light. Warm Triglyceride Assay Buffer to room temperature before use. Briefly centrifuge all small vials prior to opening.

### 3. Reagents Preparation:

Triglyceride Probe: Dissolve in 220 μl anhydrous DMSO (provided) before use. Store at −20° C., protect from light and moisture.

Triglyceride Enzyme Mix: Dissolve in 220 μl Triglyceride Assay Buffer. Aliquot and store at −20° C.

Lipase: Dissolve in 220 μl Triglyceride Assay Buffer. Aliquot and store at −20° C.

### 4. Triglyceride Assay Protocol:

4.1. Standard Curve Preparation:

Re-dissolve in hot water bath (80˜100° C.) for 1 minute or until the standard looks cloudy, vortex for 30 seconds, repeat the heat and vortex one more time. Dilute the Triglyceride Standard to 0.01 mM with the Triglyceride Assay Buffer. Add 0, 10, 20, 30, 40, 50 μl into each well individually. Adjust volume to 50 μl/well with Triglyceride Assay Buffer to generate 0.1, 0.2, 0.3, 0.4, 0.5 nmol/well of Triglyceride Standard.

4.2. Sample Preparation: Add 5 μl test samples in a 96-well clear bottom black plate, Adjust to the final volume of 50 μl/well with Triglyceride Assay Buffer.

4.3. Lipase: Add 2 μl of lipase to each standard and sample well. Mix and incubate 20 min at RT to convert triglyceride to glycerol and fatty acid.

4.4. Triglyceride Reaction Mix: Mix enough reagents for the number of samples and standards to be performed: For each well, prepare a total 50 μl Reaction Mix:

47.6 μl Triglyceride Assay Buffer

0.4 μl Triglyceride Probe

2 μl Triglyceride Enzyme Mix

4.5. Add 50 μl of the Reaction Mix to each well containing the Triglyceride Standard, test samples and controls. Mix well. Incubate at room temperature for 30 minutes, protect from light.

4.6. Measure fluorescence at Ex/Em 535/590 nm in ENSPIRE

4.7. Calculations:

Correct background by subtracting the value derived from the 0 triglyceride standard from all sample readings. Plot the standard curve. Apply sample Readings to the standard curve.

Triglyceride concentration can then be calculated:C=Ts/Sv(nmol/μl or μmol/ml or mM)

Where: Ts is triglyceride amount from standard curve (nmol).

Sv is the sample volume (before dilution) added in sample wells (μl).

### VII. Results

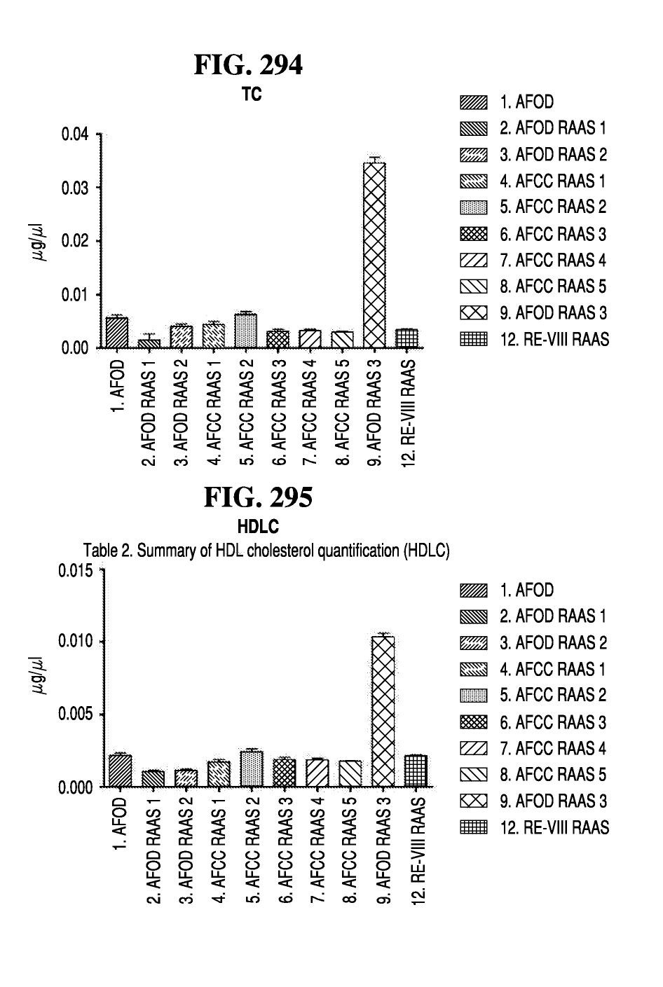

FIG. 294—Total Cholesterol/Cholesteryl Ester quantification (TC)

TABLE 11.1
Summary of Total Cholesterol/Cholesteryl Ester quantification (TC)

| | AVERAGE CONC |
|---|---|
| Test Article | (μg/μl) |
| 1. AFOD | 0.006 ± 0.0005 |
| 2. AFOD RAAS 1 | 0.001 ± 0.0011 |
| 3. AFOD RAAS 2 | 0.004 ± 0.0004 |
| 4. AFCC RAAS 1 | 0.004 ± 0.0006 |
| 5. AFCC RAAS 2 | 0.006 ± 0.0005 |
| 6. AFCC RAAS 3 | 0.003 ± 0.0004 |
| 7. AFCC RAAS 4 | 0.003 ± 0.0003 |
| 8. AFCC RAAS 5 | 0.003 ± 0.0003 |
| 9. AFOD RAAS 3 | 0.035 ± 0.0022 |
| 12. RE-VIII RAAS | 0.003 ± 0.0002 |

Normal range: human: 0.12 μg/μl~0.22 μg/μl

FIG. 295—HDL cholesterol quantification (HDLC)

TABLE 11.2
Summary of HDL cholesterol quantification
(HDLC) LIPIDS2K11-01

| | AVERAGE CONC |
|---|---|
| Test Article | (μg/μl) |
| 1. AFOD | 0.002 ± 0.0002 |
| 2. AFOD RAAS 1 | 0.001 ± 0.0001 |
| 3. AFOD RAAS 2 | 0.001 ± 0.0001 |
| 4. AFCC RAAS 1 | 0.002 ± 0.0002 |
| 5. AFCC RAAS 2 | 0.002 ± 0.0002 |
| 6. AFCC RAAS 3 | 0.002 ± 0.0002 |
| 7. AFCC RAAS 4 | 0.002 ± 0.0001 |
| 8. AFCC RAAS 5 | 0.002 ± 0.0001 |
| 9. AFOD RAAS 3 | 0.010 ± 0.0005 |
| 12. RE-VIII RAAS | 0.002 ± 0.0001 |

Normal range: human>0.03 μg/μl

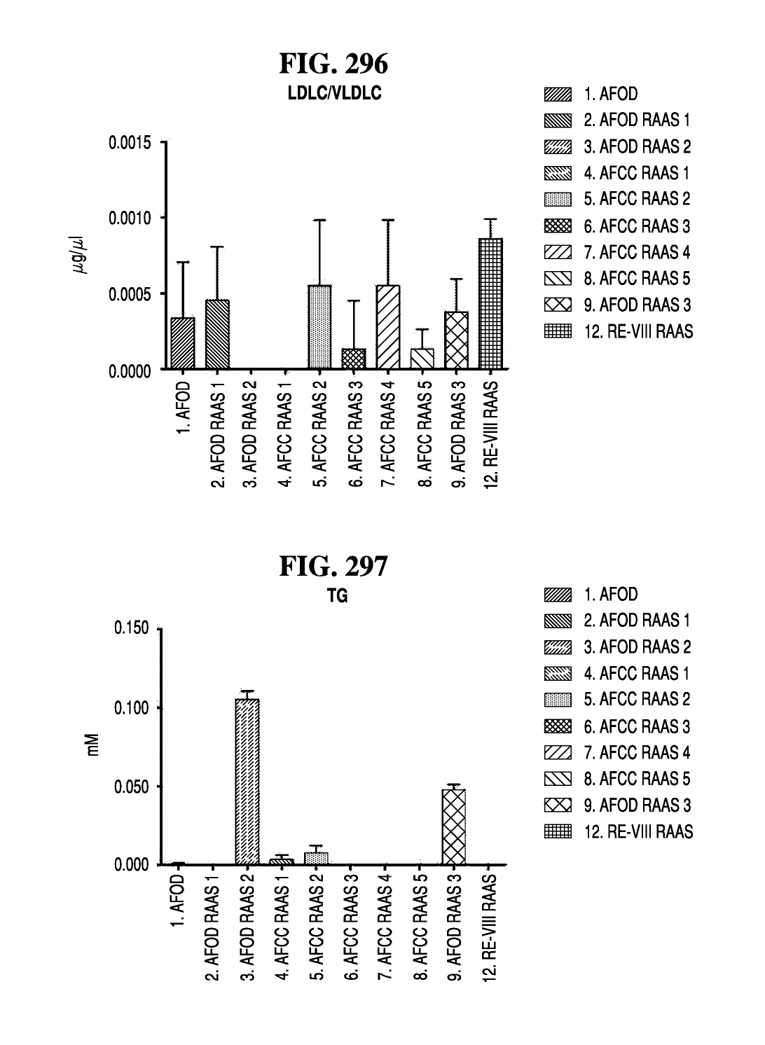

FIG. 296—LDL/VLDL cholesterol quantification (LDLC/VLDLC)

TABLE 11.3
Summary of LDL/VLDL cholesterol quantification (LDLC/VLDLC)

| Test Article | AVERAGE CONC (μg/μl) |
|---|---|
| 1. AFOD | 0.0003 ± 0.0004 |
| 2. AFOD RAAS 1 | 0.0005 ± 0.0004 |
| 3. AFOD RAAS 2 | 0.0000 ± 0.0000 |
| 4. AFCC RAAS 1 | 0.0000 ± 0.0000 |
| 5. AFCC RAAS 2 | 0.0005 ± 0.0004 |
| 6. AFCC RAAS 3 | 0.0001 ± 0.0003 |
| 7. AFCC RAAS 4 | 0.0005 ± 0.0004 |
| 8. AFCC RAAS 5 | 0.0001 ± 0.0003 |
| 9. AFOD RAAS 3 | 0.0004 ± 0.0004 |
| 12. RE-VIII RAAS | 0.0009 ± 0.0002 |

Normal range: human: 0.11 μg/μl~0.12 μg/μl

FIG. 297—Triglyceride quantification (TG)

TABLE 11.4
Summary of Triglyceride quantification (TG)

| | AVERAGE |
|---|---|
| Test Article | CONC(mM) |
| 1. AFOD | 0.001 ± 0.0006 |
| 2. AFOD RAAS 1 | 0.000 ± 0.0000 |
| 3. AFOD RAAS 2 | 0.105 ± 0.0056 |
| 4. AFCC RAAS 1 | 0.003 ± 0.0031 |
| 5. AFCC RAAS 2 | 0.007 ± 0.0045 |
| 6. AFCC RAAS 3 | 0.000 ± 0.0000 |
| 7. AFCC RAAS 4 | 0.000 ± 0.0000 |
| 8. AFCC RAAS 5 | 0.000 ± 0.0000 |
| 9. AFOD RAAS 3 | 0.047 ± 0.0073 |
| 12. RE-VIII RAAS | 0.000 ± 0.0000 |

Normal range: human: 0.45 mM~1.36 mM

As specifically requested by RAAS, the above data were re plotted individually

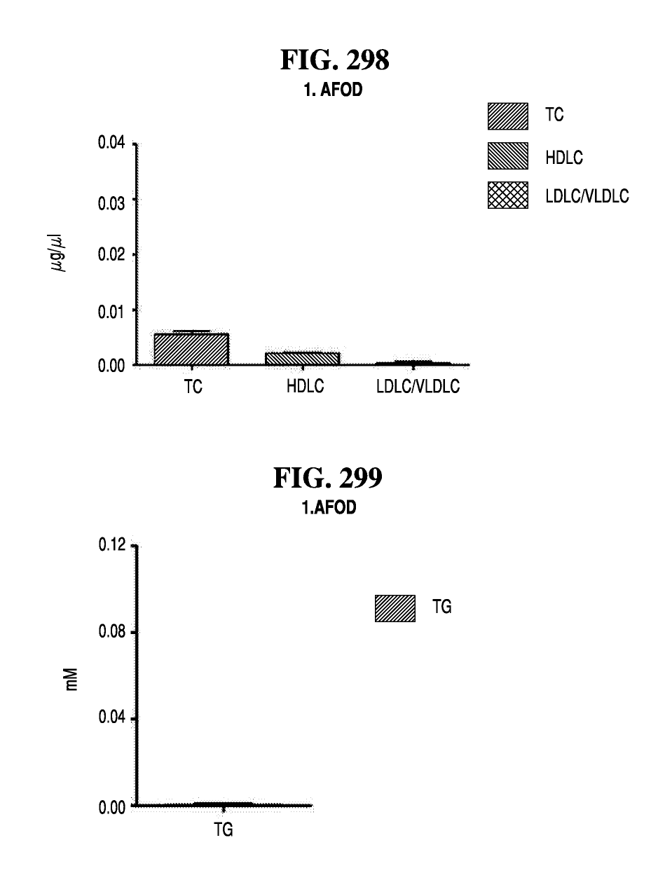

FIG. 298—TC, HDLC and LDLC/VLDLC quantification of sample #1.

### AFOD

FIG. 299—TG quantification of sample#1. AFOD

TABLE 11.5
Summary of TC, HDLC, LDLC/VLDLC and TG quantification of sample #1. AFOD

| Test Article | TC (μg/μl) | HDLC (μg/μl) | LDLC/VLDLC | TG(mM) |
|---|---|---|---|---|
| 1. AFOD | 0.006 ± 0.0005 | 0.002 ± 0.0002 | 0.0003 ± 0.0004 | 0.001 ± 0.0006 |
| Normal range | 0.12~0.22 | >0.03 | 0.11~0.12 | 0.45~1.36 |

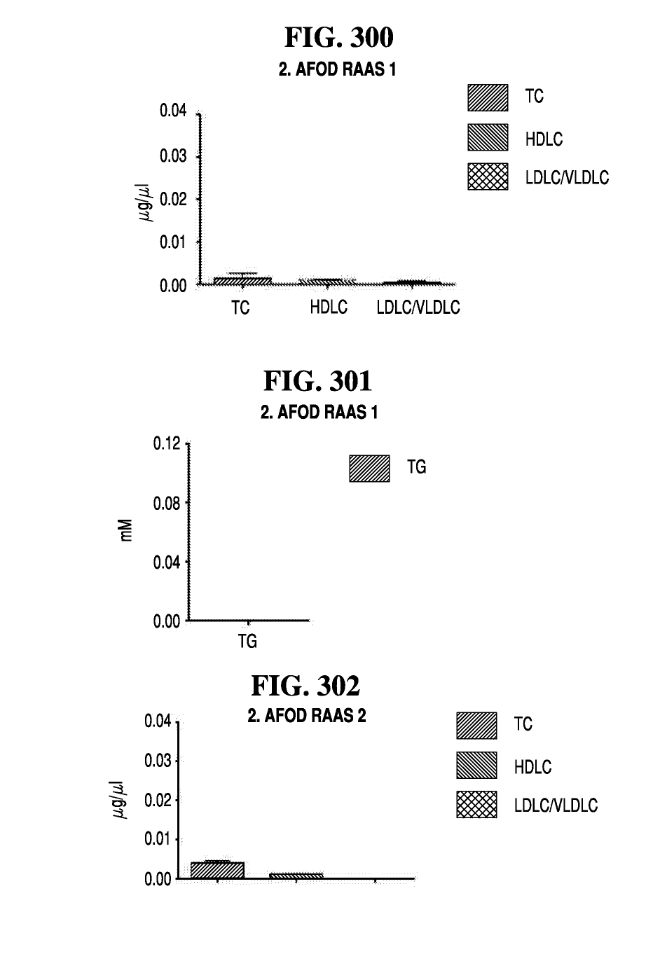

FIG. 300—TC, HDLC and LDLC/VLDLC quantification of sample #2. AFOD RAAS1

FIG. 301—TG quantification of sample #2. AFOD RAAS1

TABLE 11.6
Summary of TC, HDLC, LDLC/VLDLC and TG
Quantification of sample #2. AFOD RAAS 1

| Test Article | TC (μg/μl) | HDLC (μg/μl) | LDLC/VLDLC | TG(mM) |
|---|---|---|---|---|
| 2. AFOD RAAS | 0.001 ± 0.0011 | 0.001 ± 0.0001 | 0.0005 ± 0.0004 | 0.000 ± 0.0000 |
| Normal range | 0.12~0.22 | >0.03 | 0.11~0.12 | 0.45~1.36 |

FIG. 302—TC, HDLC and LDLC/VLDLC quantification of sample #3. AFOD RAAS2

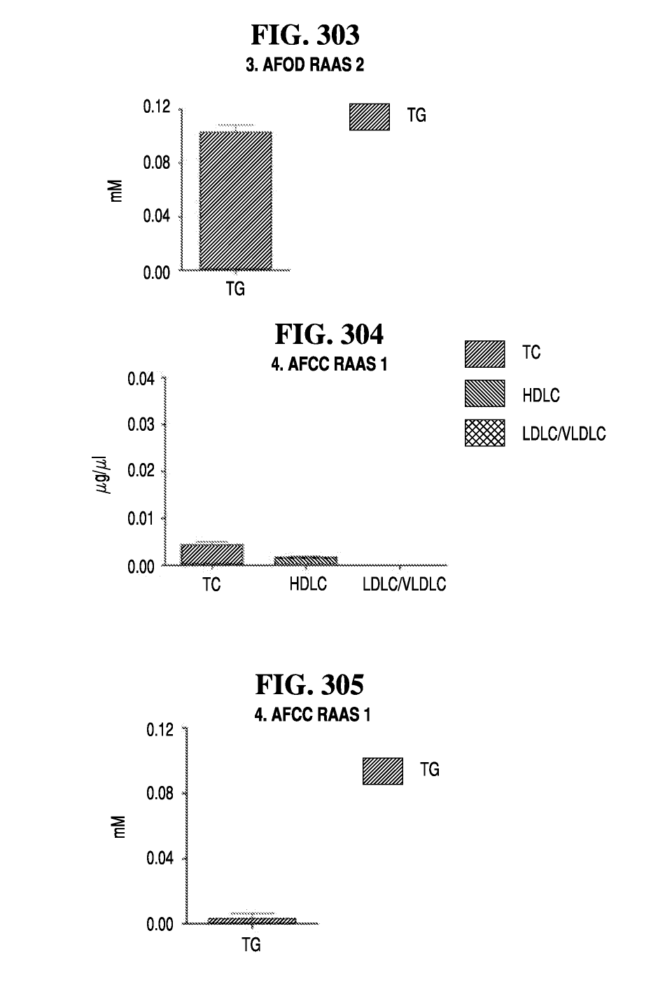

FIG. 303—TG quantification of sample #3. AFOD RAAS2

TABLE 11.7
Summary of TC, HDLC, LDLC/VLDLC and TG
quantification of sample #3. AFOD RAAS 2

| | | | LDLC/VLDLC | |
|---|---|---|---|---|
| Test Article | TC (μg/μl) | HDLC (μg/μl) | (μg/μl) | TG(mM) |
| 3. AFOD RAAS 2 | 0.004 ± 0.0004 | 0.001 ± 0.0001 | 0.0000 ± 0.0000 | 0.105 ± 0.0056 |
| Normal range | 0.12~0.22 | >0.03 | 0.11~0.12 | 0.45~1.36 |

FIG. 304—TC, HDLC and LDLC/VLDLC quantification of sample #4. AFCC RAAS 1

FIG. 305—TG quantification of sample #4. AFCC RAAS 1

TABLE 11.8
Summary of TC, HDLC, LDLC/VLDLC and TG
quantification of sample #4. AFCC RAAS 1

| | | | LDLC/VLDLC | |
|---|---|---|---|---|
| Test Article | TC (μg/μl) | HDLC (μg/μl) | (μg/μl) | TG(mM) |
| 4. AFCC RAAS 1 | 0.004 ± 0.0006 | 0.002 ± 0.0002 | 0.0000 ± 0.0000 | 0.003 ± 0.0031 |
| Normal range | 0.12~0.22 | >0.03 | 0.11~0.12 | 0.45~1.36 |

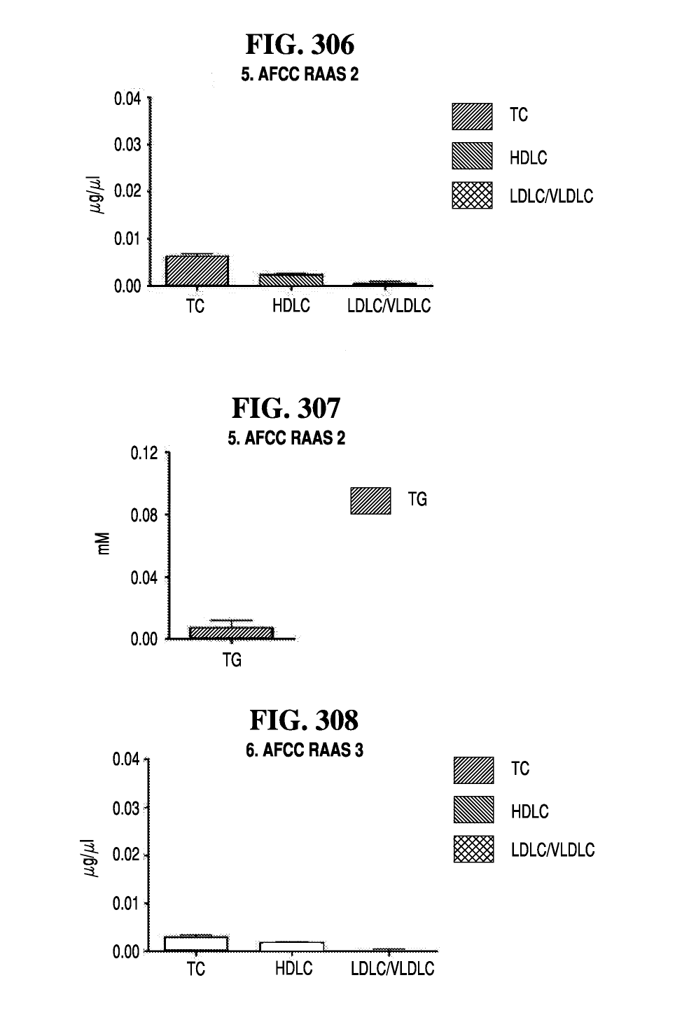

FIG. 306—TC, HDLC and LDLC/VLDLC quantification of sample #5. AFCC RAAS2

FIG. 307—TG quantification of sample #5. AFCC RAAS2

TABLE 11.9
Summary of TC, HDLC, LDLC/VLDLC and TG
Quantification of sample #5. AFCC RAAS 2

| | | | LDLC/VLDLC | |
|---|---|---|---|---|
| Test Article | TC (μg/μl) | HDLC (μg/μl) | (μg/μl) | TG(mM) |
| 5. AFCC RAAS 2 | 0.006 ± 0.0005 | 0.002 ± 0.0002 | 0.0005 ± 0.0004 | 0.007 ± 0.0045 |
| Normal range | 0.12~0.22 | >0.03 | 0.11~0.12 | 0.45~1.36 |

FIG. 308—TC, HDLC and LDLC/VLDLC quantification of sample #6. AFCC RAAS3

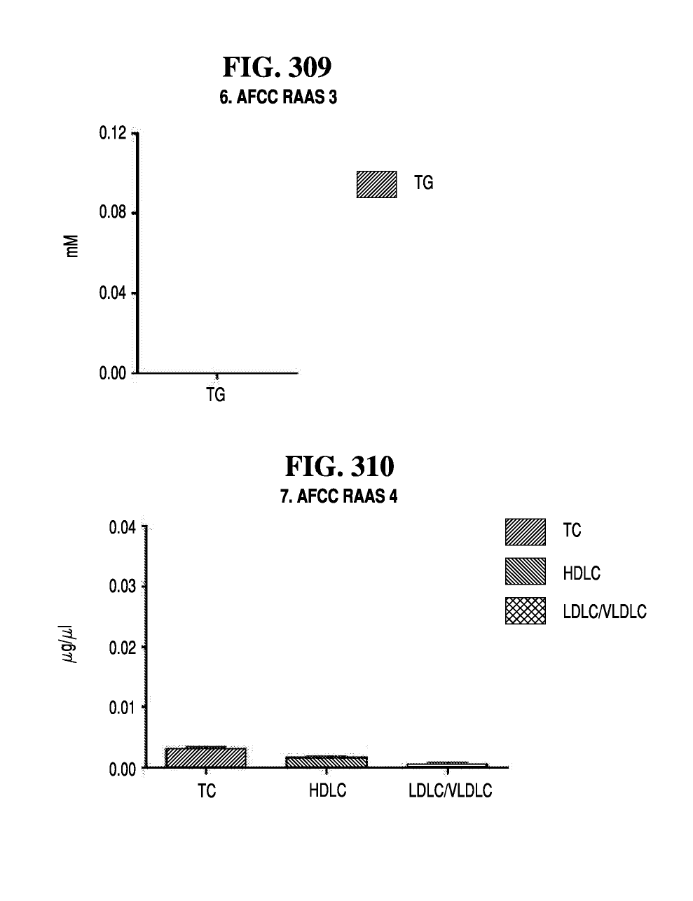

FIG. 309—TG Quantification of sample #6. AFCC RAAS3

TABLE 11.10
Summary of TC, HDLC, LDLC/VLDLC and TG
quantification of sample #6. AFCC RAAS 3

| | | | LDLC/VLDLC | |
|---|---|---|---|---|
| Test Article | TC (μg/μl) | HDLC (μg/μl) | (μg/μl) | TG(mM) |
| 6. AFCC RAAS 3 | 0.003 ± 0.0004 | 0.002 ± 0.0002 | 0.0001 ± 0.0003 | 0.000 ± 0.0000 |
| Normal range | 0.12~0.22 | >0.03 | 0.11~0.12 | 0.45~1.36 |

FIG. 310—TC, HDLC and LDLC/VLDLC quantification of sample #7. AFCC RAAS4

FIG. 311—TG quantification of sample #7. AFCC RAAS4

TABLE 11.11
Summary of TC, HDLC, LDLC/VLDLC and TG
Quantification of sample #7. AFCC RAAS 4

| | | | LDLC/VLDLC | |
|---|---|---|---|---|
| Test Article | TC (μg/μl) | HDLC (μg/μl) | (μg/μl) | TG(mM) |
| 7. AFCC RAAS 4 | 0.003 ± 0.0003 | 0.002 ± 0.0001 | 0.0005 ± 0.0004 | 0.000 ± 0.0000 |
| Normal range | 0.12~0.22 | >0.03 | 0.11~0.12 | 0.45~1.36 |

FIG. 312—TC, HDLC and LDLC/VLDLC quantification of sample #8. AFCC RAAS5

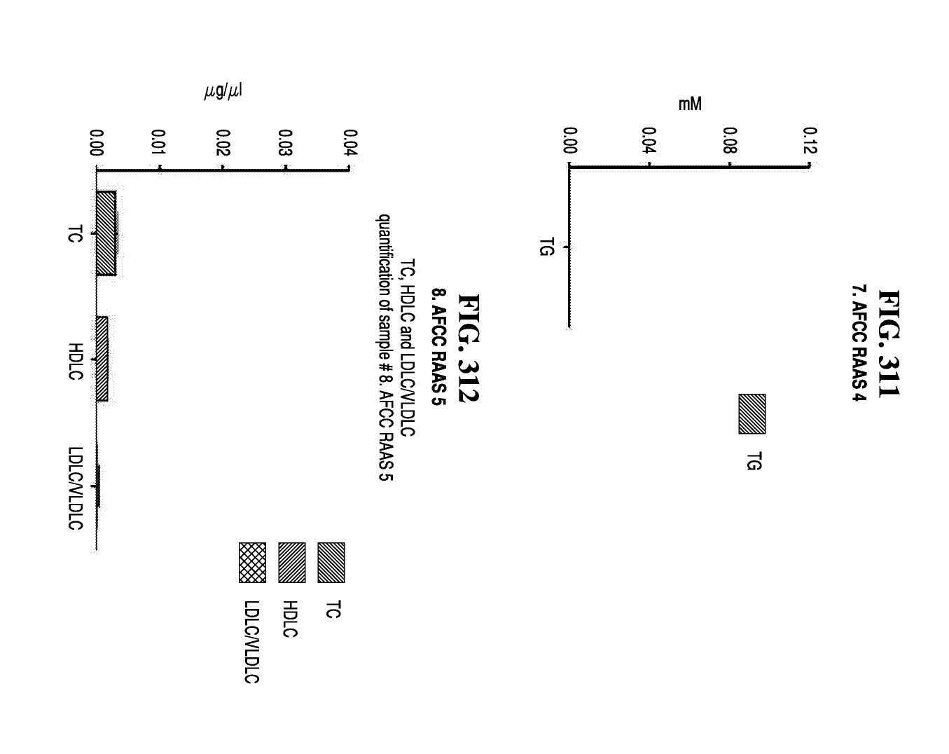

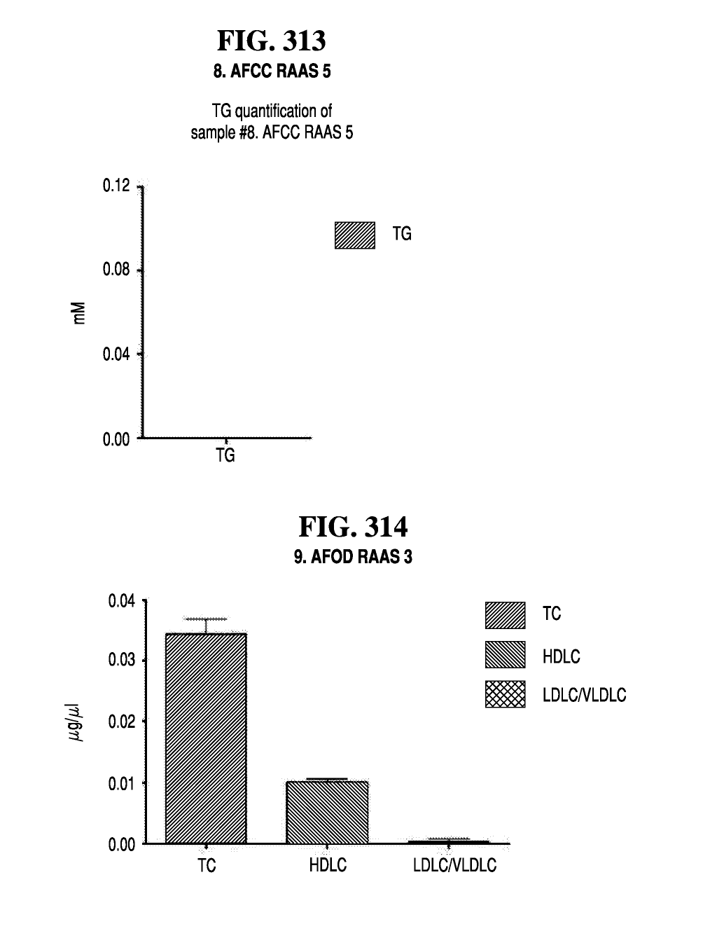

FIG. 313—TG quantification of sample #8. AFCC RAAS5

TABLE 11.12
Summary of TC, HDLC, LDLC/VLDLC and TG
quantification of sample #8.
AFCC RAAS 5

| | | | LDLC/ | |
|---|---|---|---|---|
| | TC | HDLC | VLDLC | |
| Test Article | (μg/μl) | (μg/μl) | (μg/μl) | TG(mM) |
| 8. AFCC RAAS 5 | 0.003 ± | 0.002 ± | 0.0001 ± | 0.000 ± |
| | 0.0003 | 0.0001 | 0.0003 | 0.0000 |
| Normal range | 0.12~0.22 | >0.03 | 0.11~0.12 | 0.45~1.36 |

FIG. 314—TC, HDLC and LDLC/VLDLC quantification of sample #9. AFOD RAAS3

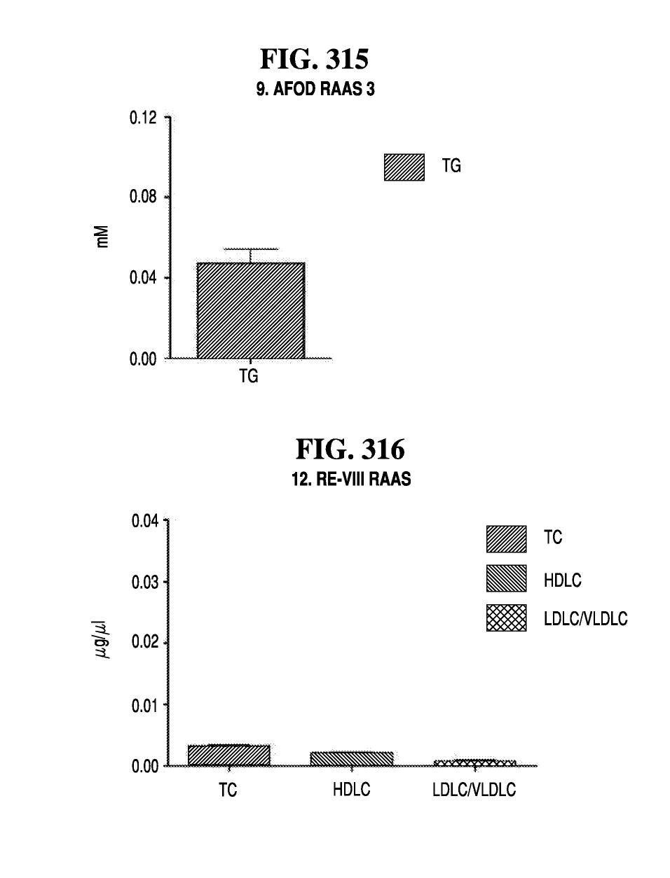

FIG. 315—TG quantification of sample #9. AFOD RAAS3

TABLE 11.13
Summary of TC, HDLC, LDLC/VLDLC and TG
quantification of sample #9.
AFOD RAAS 3

| | TC | HDLC | LDLC/ | |
|---|---|---|---|---|
| Test Article | (μg/μl) | (μg/μl) | VLDLC | TG(mM) |
| 9. AFOD RAAS 3 | 0.035 ± | 0.010 ± | 0.0004 ± | 0.047 ± |
| | 0.0022 | 0.0005 | 0.0004 | 0.0073 |
| Normal range | 0.12~0.22 | >0.03 | 0.11~0.12 | 0.45~1.36 |

FIG. 316—TC, HDLC and LDLC/VLDLC quantification of sample #12. RE-VIII RAAS

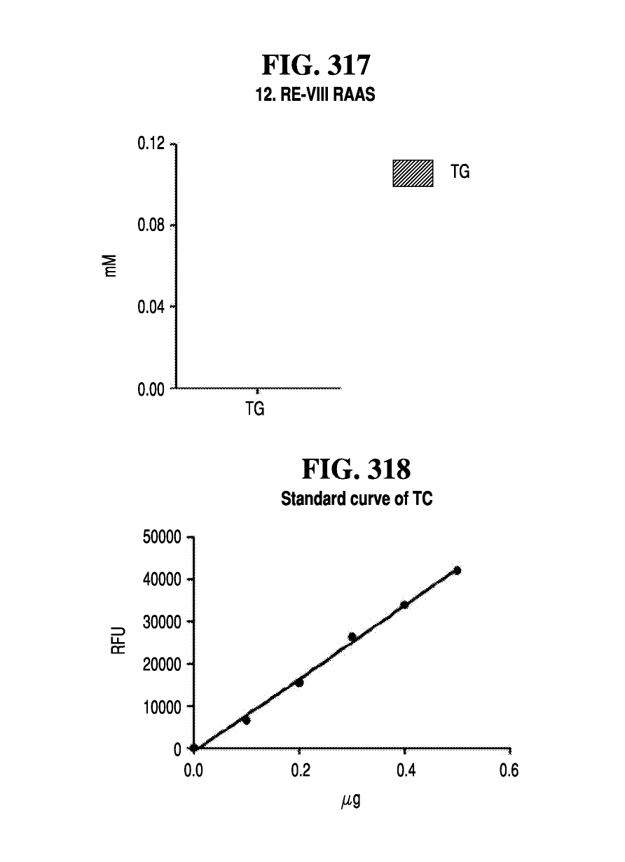

FIG. 317—TG quantification of sample #12. RE-VIII RAAS

TABLE 11.14
Summary of TC, HDLC, LDLC/VLDLC and TG
quantification in sample #12.
RE-VIII RAAS

| | TC | HDLC | LDLC/ | |
|---|---|---|---|---|
| Test Article | (μg/μl) | (μg/μl) | VLDLC | TG(mM) |
| 12. RE-VIII | 0.003 ± | 0.002 ± | 0.0009 ± | 0.000 ± |
| | 0.0002 | 0.0001 | 0.0002 | 0.0000 |
| Normal range | 0.12~0.22 | >0.03 | 0.11~0.12 | 0.45~1.36 |

### VIII. Conclusion

1. All selected RAAS products were tested duplicated for data accuracy. All the RAAS samples have no or very low detectable level of lipids.

2. AFOD RAAS2 and AFOD RAAS3 have a low concentration of TG which are a little higher than the other samples.

3. AFOD RAAS3 has a low concentration of TC and HDLC that is higher than the other samples.

### IX. Raw Data

TABLE 11.15
Raw data of Total Cholesterol/Cholesteryl Ester Quantification (TC)

| | | | DILUTION 1 | DILUTION 2 | AVERAGE |
|---|---|---|---|---|---|
| | AVERAGE | VOLUME | CONC | CONC | CONC |
| | (RFU) | (μl) | (μg/μl) | (μg/μl) | (μg/μl) |
| STD 0 μg | 3273 | | | | |
| STD 0.1 μg | 9871 | | | | |
| STD 0.2 μg | 18824 | | | | |
| STD 0.3 μg | 29598 | | | | |
| STD 0.4 μg | 37139 | | | | |
| STD 0.5 μg | 45302 | | | | |
| 1.1 AFOD | 4961 | 5 | 0.006 | 0.005 | 0.006 |
| 1.2 AFOD | 4789 | 5 | 0.006 | 0.005 | 0.005 |
| 1.3 AFOD | 4940 | 5 | 0.006 | 0.006 | 0.006 |
| 2.1 AFOD RAAS1 | 3469 | 5 | 0.002 | 0.002 | 0.002 |
| 2.2 AFOD RAAS1 | 3270 | 5 | 0.000 | 0.002 | 0.001 |
| 2.3 AFOD RAAS1 | 3326 | 5 | 0.002 | 0.000 | 0.001 |
| 3.1 AFOD RAAS2 | 4124 | 5 | 0.004 | 0.004 | 0.004 |
| 3.2 AFOD RAAS2 | 4375 | 5 | 0.005 | 0.004 | 0.004 |
| 3.3 AFOD RAAS2 | 4140 | 5 | 0.004 | 0.004 | 0.004 |
| 4.1 AFCC RAAS1 | 4479 | 5 | 0.004 | 0.005 | 0.005 |
| 4.2 AFCC RAAS1 | 4320 | 5 | 0.005 | 0.004 | 0.004 |
| 4.3 AFCC RAAS1 | 4280 | 5 | 0.005 | 0.004 | 0.004 |
| 5.1 AFCC RAAS2 | 5286 | 5 | 0.007 | 0.007 | 0.007 |
| 5.2 AFCC RAAS2 | 5238 | 5 | 0.006 | 0.007 | 0.006 |
| 5.3 AFCC RAAS2 | 4992 | 5 | 0.006 | 0.005 | 0.006 |
| 6.1 AFCC RAAS3 | 3918 | 5 | 0.004 | 0.003 | 0.003 |
| 6.2 AFCC RAAS3 | 3899 | 5 | 0.003 | 0.003 | 0.003 |
| 6.3 AFCC RAAS3 | 3649 | 5 | 0.003 | 0.003 | 0.003 |
| 7.1 AFCC RAAS4 | 3928 | 5 | 0.004 | 0.003 | 0.003 |
| 7.2 AFCC RAAS4 | 3745 | 5 | 0.003 | 0.003 | 0.003 |
| 7.3 AFCC RAAS4 | 3923 | 5 | 0.003 | 0.003 | 0.003 |
| 8.1 AFCC RAAS5 | 3758 | 5 | 0.003 | 0.003 | 0.003 |
| 8.2 AFCC RAAS5 | 3705 | 5 | 0.003 | 0.003 | 0.003 |
| 8.3 AFCC RAAS5 | 3832 | 5 | 0.003 | 0.004 | 0.003 |
| 9.1 AFOD RAAS3 | 16975 | 5 | 0.032 | 0.036 | 0.034 |
| 9.2 AFOD RAAS3 | 17497 | 5 | 0.037 | 0.033 | 0.035 |
| 12.1 RE-VIII RAAS | 3951 | 5 | 0.003 | 0.004 | 0.003 |

FIG. 318—Standard curve of Total Cholesterol/Cholesterol Ester Quantification (TC)

TABLE 11.16
Raw data of HDL Cholesterol Quantification (HDLC)

| | | | DILUTION 1 | DILUTION 2 | AVERAGE |
|---|---|---|---|---|---|
| | AVERAGE | VOLUME | CONC | CONC | CONC |
| | (RFU) | (μl) | (μg/μl) | (μg/μl) | (μg/μl) |
| STD 0 μg | 4193 | | | | |
| STD 0.1 μg | 14765 | | | | |
| STD 0.2 μg | 28156 | | | | |
| STD 0.3 μg | 42490 | | | | |
| STD 0.4 μg | 52471 | | | | |
| STD 0.5 μg | 66055 | | | | |
| 1.1 AFOD | 6208 | 5 | 0.002 | 0.002 | 0.002 |
| 1.2 AFOD | 6199 | 5 | 0.002 | 0.002 | 0.002 |
| 1.3 AFOD | 6366 | 5 | 0.002 | 0.002 | 0.002 |
| 2.1 AFOD RAAS1 | 4886 | 5 | 0.001 | 0.001 | 0.001 |
| 2.2 AFOD RAAS1 | 4808 | 5 | 0.001 | 0.001 | 0.001 |
| 2.3 AFOD RAAS1 | 4907 | 5 | 0.001 | 0.001 | 0.001 |
| 3.1 AFOD RAAS2 | 4949 | 5 | 0.001 | 0.001 | 0.001 |
| 3.2 AFOD RAAS2 | 4998 | 5 | 0.001 | 0.001 | 0.001 |
| 3.3 AFOD RAAS2 | 4974 | 5 | 0.001 | 0.001 | 0.001 |
| 4.1 AFCC RAAS1 | 5583 | 5 | 0.002 | 0.002 | 0.002 |
| 4.2 AFCC RAAS1 | 5815 | 5 | 0.002 | 0.002 | 0.002 |
| 4.3 AFCC RAAS1 | 5689 | 5 | 0.002 | 0.002 | 0.002 |
| 5.1 AFCC RAAS2 | 6527 | 5 | 0.002 | 0.002 | 0.002 |
| 5.2 AFCC RAAS2 | 6813 | 5 | 0.003 | 0.003 | 0.003 |
| 5.3 AFCC RAAS2 | 6427 | 5 | 0.002 | 0.002 | 0.002 |
| 6.1 AFCC RAAS3 | 6108 | 5 | 0.002 | 0.002 | 0.002 |
| 6.2 AFCC RAAS3 | 5840 | 5 | 0.002 | 0.002 | 0.002 |
| 6.3 AFCC RAAS3 | 5732 | 5 | 0.002 | 0.002 | 0.002 |
| 7.1 AFCC RAAS4 | 5775 | 5 | 0.002 | 0.002 | 0.002 |
| 7.2 AFCC RAAS4 | 6009 | 5 | 0.002 | 0.002 | 0.002 |
| 7.3 AFCC RAAS4 | 5850 | 5 | 0.002 | 0.002 | 0.002 |
| 8.1 AFCC RAAS5 | 5794 | 5 | 0.002 | 0.002 | 0.002 |
| 8.2 AFCC RAAS5 | 5724 | 5 | 0.002 | 0.002 | 0.002 |
| 8.3 AFCC RAAS5 | 5747 | 5 | 0.002 | 0.002 | 0.002 |
| 9.1 AFOD RAAS3 | 16875 | 5 | 0.011 | 0.010 | 0.011 |
| 9.2 AFOD RAAS3 | 15875 | 5 | 0.010 | 0.010 | 0.010 |
| 12.1 RE-VIII RAAS | 6239 | 5 | 0.002 | 0.002 | 0.002 |

FIG. 319—Standard curve of HDL Cholesterol Quantification (HDLC)

TABLE 11.17
Raw data of LDL/VLDL Cholesterol Quantification (LDLC/VLDLC)

| | | | | DILUTION 1 | DILUTION 2 | VERAGE |
|---|---|---|---|---|---|---|
| | | [symbol] VERAGE | [symbol] LUME | CONC | CONC | CONC |
| | | (RFU) | (μl) | (μg/μl) | (μg/μl) | (μg/μl) |
| STD | 0 μg | 5485 | | | | |
| STD | 0.1 μg | 17372 | | | | |
| STD | 0.2 μg | 33613 | | | | |
| STD | 0.3 μg | 45559 | | | | |
| STD | 0.4 μg | 58281 | | | | |
| STD | 0.5 μg | 67440 | | | | |
| 1.1 | AFOD | 5428 | 5 | 0.001 | 0.000 | 0.000 |
| 1.2 | AFOD | 5559 | 5 | 0.001 | 0.001 | 0.001 |
| 1.3 | AFOD | 5406 | 5 | 0.000 | 0.000 | 0.000 |
| 2.1 | AFOD RAAS1 | 5161 | 5 | 0.000 | 0.000 | 0.000 |
| 2.2 | AFOD RAAS1 | 5559 | 5 | 0.001 | 0.001 | 0.001 |
| 2.3 | AFOD RAAS1 | 5626 | 5 | 0.001 | 0.001 | 0.001 |
| 3.1 | AFOD RAAS2 | 3456 | 5 | 0.000 | 0.000 | 0.000 |
| 3.2 | AFOD RAAS2 | 3501 | 5 | 0.000 | 0.000 | 0.000 |
| 3.3 | AFOD RAAS2 | 3433 | 5 | 0.000 | 0.000 | 0.000 |
| 4.1 | AFCC RAAS1 | 5030 | 5 | 0.000 | 0.000 | 0.000 |
| 4.2 | AFCC RAAS1 | 5347 | 5 | 0.000 | 0.000 | 0.000 |
| 4.3 | AFCC RAAS1 | 5111 | 5 | 0.000 | 0.000 | 0.000 |
| 5.1 | AFCC RAAS2 | 5885 | 5 | 0.001 | 0.001 | 0.001 |
| 5.2 | AFCC RAAS2 | 5728 | 5 | 0.001 | 0.001 | 0.001 |
| 5.3 | AFCC RAAS2 | 5288 | 5 | 0.000 | 0.000 | 0.000 |
| 6.1 | AFCC RAAS3 | 5327 | 5 | 0.000 | 0.000 | 0.000 |
| 6.2 | AFCC RAAS3 | 5396 | 5 | 0.000 | 0.000 | 0.000 |
| 6.3 | AFCC RAAS3 | 5601 | 5 | 0.000 | 0.001 | 0.000 |
| 7.1 | AFCC RAAS4 | 5758 | 5 | 0.001 | 0.001 | 0.001 |
| 7.2 | AFCC RAAS4 | 5727 | 5 | 0.001 | 0.001 | 0.001 |
| 7.3 | AFCC RAAS4 | 5944 | 5 | 0.000 | 0.000 | 0.000 |
| 8.1 | AFCC RAAS5 | 5675 | 5 | 0.000 | 0.000 | 0.000 |
| 8.2 | AFCC RAAS5 | 5651 | 5 | 0.000 | 0.000 | 0.000 |
| 8.3 | AFCC RAAS5 | 5698 | 5 | 0.000 | 0.001 | 0.000 |
| 9.1 | AFOD RAAS3 | 5316 | 5 | 0.000 | 0.000 | 0.000 |
| 9.2 | AFOD RAAS3 | 5698 | 5 | 0.001 | 0.001 | 0.001 |
| 12.1 | RE-VIII RAAS | 5857 | 5 | 0.001 | 0.001 | 0.001 |

[symbol]

indicates data missing or illegible when filed

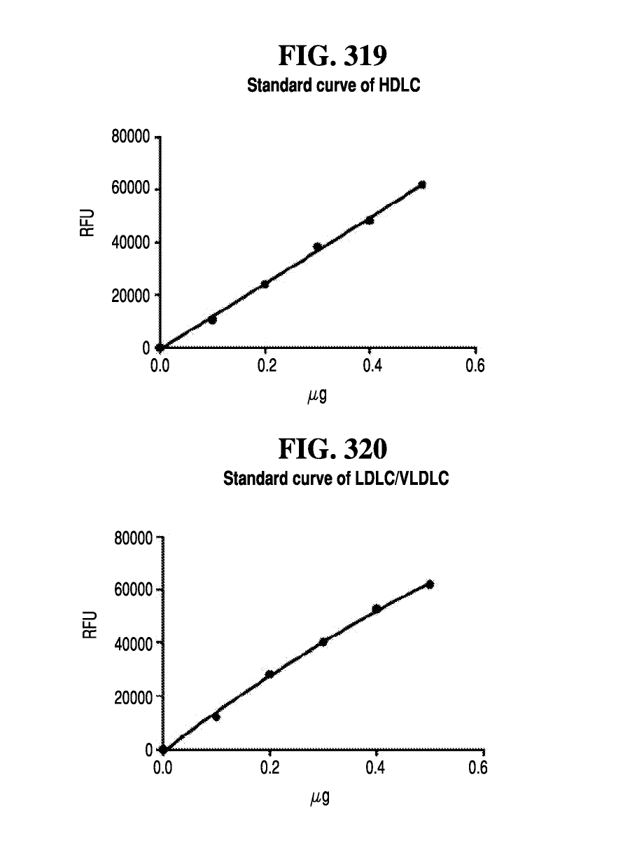

FIG. 320—Standard curve of LDL/VLDL Cholesterol Quantification (LDLC/VLDLC)

| | AVERAGE | VOLUME | DILUTION 1 | DILUTION 2 | |
|---|---|---|---|---|---|
| | (RFU) | (μl) | CONC (mM) | AVERAGE CONC (mM) | |
| STD 0 nmol | 47526 | | | CONC (mM) | |
| STD 0.1 nmol | 51671 | | | | |
| STD 0.2 nmol | 57288 | | | | |
| STD 0.3 nmol | 63212 | | | | |
| STD 0.4 nmol | 70295 | | | | |
| STD 0.5 nmol | 74368 | | | | |
| *1.1 AFOD | 47365 | 5 | 0.001 | — | 0.001 |
| *1.2 AFOD | 47173 | 5 | 0.000 | — | 0.000 |
| *1.3 AFOD | 45856 | 5 | 0.000 | — | 0.000 |
| *2.1 AFOD RAAS1 | 24562 | 5 | 0.000 | — | 0.000 |
| *2.2 AFOD RAAS1 | 24780 | 5 | 0.000 | — | 0.000 |
| *2.3 AFOD RAAS1 | 24347 | 5 | 0.000 | — | 0.000 |
| 3.1 AFOD RAAS2 | 78421 | 5 | 0.114 | 0.108 | 0.111 |
| 3.2 AFOD RAAS2 | 76371 | 5 | 0.104 | 0.105 | 0.104 |
| 3.3 AFOD RAAS2 | 74825 | 5 | 0.099 | 0.100 | 0.099 |
| 4.1 AFCC RAAS1 | 48773 | 5 | 0.005 | 0.008 | 0.007 |
| 4.2 AFCC RAAS1 | 47578 | 5 | 0.003 | 0.001 | 0.002 |
| 4.3 AFCC RAAS1 | 47032 | 5 | 0.002 | 0.000 | 0.001 |
| 5.1 AFCC RAAS2 | 49547 | 5 | 0.010 | 0.009 | 0.010 |
| 5.2 AFCC RAAS2 | 49817 | 5 | 0.011 | 0.011 | 0.011 |
| 5.3 AFCC RAAS2 | 47329 | 5 | 0.003 | 0.000 | 0.002 |
| 6.1 AFCC RAAS3 | 43219 | 5 | 0.000 | 0.000 | 0.000 |
| 6.2 AFCC RAAS3 | 42098 | 5 | 0.000 | 0.000 | 0.000 |
| 6.3 AFCC RAAS3 | 41083 | 5 | 0.000 | 0.000 | 0.000 |
| 7.1 AFCC RAAS4 | 46554 | 5 | 0.000 | 0.000 | 0.000 |
| 7.2 AFCC RAAS4 | 44498 | 5 | 0.000 | 0.000 | 0.000 |
| 7.3 AFCC RAAS4 | 43419 | 5 | 0.000 | 0.000 | 0.000 |
| 8.1 AFCC RAAS5 | 38654 | 5 | 0.000 | 0.000 | 0.000 |
| 8.2 AFCC RAAS5 | 38141 | 5 | 0.000 | 0.000 | 0.000 |
| 8.3 AFCC RAAS5 | 38136 | 5 | 0.055 | 0.045 | 0.050 |
| 9.1 AFOD RAAS3 | 60454 | 5 | 0.051 | 0.039 | 0.045 |
| 9.2 AFOD RAAS3 | 59038 | 5 | 0.000 | 0.000 | 0.000 |
| 12.1 RE-VIII RAAS | 40516 | 5 | 0.000 | 0.000 | 0.000 |

*No data was generated from Dilution 2 of 1.AFOD and 2.AFOD RAAS1 because of no enough reagents.

FIG. 321—Standard curve of Triglyceride Quantification (TG)

Quantification of Cholesterol and Triglyceride Levels in RAAS Products

### I. General Information

| | |
|---|---|
| Experiment requested by: | Mr. Kieu Hoang from Shanghai RAAS |
| Project ID/code: | LIPIDS/T01 |
| Experimental objective: | Lipids panel tests (TC, TG, HDL and LDL) |
| | by Biovision kits |
| Experiment NO: | LIPIDS2K12-02 |

### II. Introduction

The objective of this study was to quantify Cholesterol/Cholesteryl Ester (TC), HDL Cholesterol (HDLC) LDL/VLDL Cholesterol (LDLC/VLDLC) and Triglyceride (TG) concentration in RAAS products.
The Cholesterol/Cholesteryl Ester Quantitation Kit provides a simple method for sensitive quantification of free cholesterol, cholesteryl esters, or both by colorimetric or fluorometric methods. Majority of the cholesterol in blood is in the form of cholesteryl esters which can be hydrolyzed to cholesterol by cholesterol esterase. Cholesterol is then oxidized by cholesterol oxidase to yield H₂O₂ which reacts with a sensitive cholesterol probe to produce color (λmax=570 nm) and fluorescence (Ex/Em=535/590 nm). The assay detects total cholesterol (cholesterol and cholesteryl esters) in the presence of cholesterol esterase or free cholesterol in the absence of cholesterol esterase in the reaction.
BioVision's HDL and LDL/VLDL Cholesterol Quantification Kit provides a simple quantification method of HDL and LDL/VLDL after a convenient separation of HDL from LDL and VLDL (very low-density lipoprotein) in serum samples. In the assay, cholesterol oxidase specifically recognizes free cholesterol and produces products which react with probe to generate color (X=570 nm) and fluorescence (Ex/Em=538/587 nm). Cholesterol esterase hydrolizes cholesteryl ester into free cholesterol, therefore, cholesterol ester and free cholesterol can be detected separately in the presence and absence of cholesterol esterase in the reactions.
The Triglyceride Quantification Kit provides a sensitive, easy assay to measure triglyceride concentration in variety of samples. In the assay, triglycerides are converted to free fatty acids and glycerol. The glycerol is then oxidized to generate a product which reacts with the probe to generate colorimetric (spectrophotometry at λ=570 nm) and fluorometric (Ex/Em=535/590 nm) methods. The kit can detect 1 pmol-10 nmol (or 1˜10000 μM range) of triglyceride in various samples.

III. Sample list

| | Volume | Sample Preparation | Storage |
|---|---|---|---|
| KH 101 | ~1 ml | use as supplied | −20° C. |
| KH 102 | ~1 ml | use as supplied | −20° C. |
| KH 103 | ~1 ml | use as supplied | −20° C. |
| KH 104 | ~1 ml | use as supplied | −20° C. |
| KH 105 | ~1 ml | use as supplied | −20° C. |
| KH 106 | ~1 ml | use as supplied | −20° C. |
| KH 107 | ~1 ml | use as supplied | −20° C. |
| KH 108 | ~1 ml | use as supplied | −20° C. |
| KH 109 | ~1 ml | use as supplied | −20° C. |
| KH 110 | ~1 ml | use as supplied | −20° C. |
| KH 111 | ~1 ml | use as supplied | −20° C. |
| KH 112 | ~1 ml | use as supplied | −20° C. |
| KH 113 | ~1 ml | use as supplied | −20° C. |
| KH 114 | ~1 ml | use as supplied | −20° C. |
| KH 115 | ~1 ml | use as supplied | −20° C. |
| KH 116 | ~1 ml | use as supplied | −20° C. |
| KH 117 | ~1 ml | use as supplied | −20° C. |
| KH 118 | ~1 ml | use as supplied | −20° C. |
| KH 119 | ~1 ml | use as supplied | −20° C. |
| KH 120 | ~1 ml | use as supplied | −20° C. |
| KH 121 | ~1 ml | use as supplied | −20° C. |
| KH 122 | ~1 ml | use as supplied | −20° C. |
| KH 123 | ~1 ml | use as supplied | −20° C. |
| KH 124 | ~1 ml | use as supplied | −20° C. |
| KH 125 | ~1 ml | use as supplied | −20° C. |
| KH 126 | ~1 ml | use as supplied | −20° C. |
| KH 127 | ~1 ml | use as supplied | −20° C. |
| KH 128 | ~1 ml | use as supplied | −20° C. |
| KH 129 | ~1 ml | use as supplied | −20° C. |
| KH 130 | ~1 ml | use as supplied | −20° C. |
| KH 131 | ~1 ml | use as supplied | −20° C. |
| KH 132 | ~1 ml | use as supplied | −20° C. |
| KH 133 | ~1 ml | use as supplied | −20° C. |
| KH 134 | ~1 ml | use as supplied | −20° C. |
| KH 201 | ~1 ml | use as supplied | −20° C. |
| KH 202 | ~1 ml | use as supplied | −20° C. |
| KH 203 | ~1 ml | use as supplied | −20° C. |
| KH 204 | ~1 ml | use as supplied | −20° C. |
| KH 205 | ~1 ml | use as supplied | −20° C. |
| KH 206 | ~1 ml | use as supplied | −20° C. |
| KH 208 | ~1 ml | use as supplied | −20° C. |
| KH 209 | ~1 ml | use as supplied | −20° C. |
| KH 210 | ~1 ml | use as supplied | −20° C. |
| KH 211 | ~1 ml | use as supplied | −20° C. |
| KH 212 | ~1 ml | use as supplied | −20° C. |
| KH 213 | ~1 ml | use as supplied | −20° C. |
| KH 214 | ~1 ml | use as supplied | −20° C. |
| KH 215 | ~1 ml | use as supplied | −20° C. |
| KH 216 | ~1 ml | use as supplied | −20° C. |
| KH 217 | ~1 ml | use as supplied | −20° C. |
| KH 301 | ~1 ml | use as supplied | −20° C. |
| KH 302 | ~1 ml | use as supplied | −20° C. |
| KH 303 | ~1 ml | use as supplied | −20° C. |
| KH 304 | ~1 ml | use as supplied | −20° C. |
| KH 305 | ~1 ml | use as supplied | −20° C. |
| KH 306 | ~1 ml | use as supplied | −20° C. |
| KH 307 | ~1 ml | use as supplied | −20° C. |
| KH 308 | ~1 ml | use as supplied | −20° C. |
| KH 309 | ~1 ml | use as supplied | −20° C. |

### IV. Total Cholesterol/Cholesteryl Ester Quantification by Fluorometric Method (TC)

Cholesterol/Cholesteryl Ester Quantitation Kit (Catalog #K603-100; 100 assays; Store at −20° C.)

### 1. Kit Contents:

| Components | K622-100 | Cap Code | Part Number |
|---|---|---|---|
| Cholesterol Assay Buffer | 25 ml | WM | K603-100-1 |
| Cholesterol Probe (in DMSO, | 200 μl | Red | K603-100-2A |
| anhydrous) | | | |
| Enzyme Mix (lyophilized) | 1 vial | Green | K603-100-4 |
| Cholesterol Esterase (lyophilized) | 1 vial | Blue | K603-100-5 |
| Cholesterol Standard (2 μg/μl) | 100 μl | Yellow | K603-100-6 |

### 2. Storage and Handling:

Store kit at −20° C., protect from light. Warm to room temperature before use. Keep enzymes and cholesterol standard on ice while using.

### 3. Reagents Preparation:

Cholesterol Probe: Warm to room temperature to thaw the DMSO solution before use. Store at −20° C., protect from light.

Cholesterol Esterase: Dissolve in 220 μl Cholesterol Assay Buffer before use. Aliquot and store at −20° C.

Enzyme Mix: Dissolve in 220 μl Cholesterol Assay Buffer before use. Aliquot and store at −20° C.

### 4. Cholesterol Assay Protocol:

4.1. Standard Curve Preparation:

Dilute the Cholesterol Standard to 25 ng/μl by adding 10 μl of the Cholesterol Standard to 790 μl of Cholesterol Assay Buffer, mix well. Add 0, 4, 8, 12, 16, 20 μl into a series of wells. Adjust volume to 50 μl/well with Cholesterol Assay Buffer to generate 0, 0.1, 0.2, 0.3, 0.4, 0.5 μg/well of the Cholesterol Standard.

4.2. Sample Preparation: Add 5 μl test samples in a 96-well clear bottom black plate, Adjust to the final volume of 50 μl/well with Cholesterol Assay Buffer.

4.3. Cholesterol Reaction Mix: Mix enough reagents for the number of samples and standards to be performed: For each well, prepare a total 50 μl Reaction Mix:
45.6 μl Cholesterol Assay Buffer
0.4 μl Cholesterol Probe
2 μl Cholesterol Enzyme Mix
2 μl Cholesterol Esterase

4.4. Mix well Add 50 μl of the Reaction Mix to each well containing standard or test samples.

4.5. Incubate the reaction for 60 minutes at 37° C., protect from light.

4.6. Measure fluorescence at Ex/Em 535/590 nm in ENSPIRE

4.7. Calculations: Subtract 0 standard reading from readings. Plot the standard curve. Apply the sample readings to the standard curve to determine sample cholesterol amount in the reaction well.

### Sample Cholesterol Concentrations:

C=A/V(μg/μl)

Where: A is the sample cholesterol amount from the standard curve (μg).

V is original sample volume added to the sample reaction well (μl).

### V. HDL and LDL/VLDL Cholesterol Quantification by Fluorometric Method (HDLC and LDLC/VLDLC)

HDL and LDL&VLDL Cholesterol Quantification Kit (Catalog #K613-100; 100 assays; Store at −20° C.)

### 1. Kit Contents:

| Components | Volume | Cap Code | Part No. |
|---|---|---|---|
| Cholesterol Assay Buffer | 25 ml | WM | K613-100-1 |
| 2× LDL/VLDL Precipitation Buffer | 10 ml | NM | K613-100-2 |
| Cholesterol Probe | 200 μl | Red | K613-100-3A |
| (in DMSO, anhydrous) | | | |
| Enzyme Mix (Lyophilized) | 1 vial | Green | K613-100-5 |
| Cholesterol Esterase (Lyophilized) | 1 vial | Blue | K613-100-6 |
| Cholesterol Standard (2 μg/μl) | 100 μl | Yellow | K613-100-7 |

### 2. Reagent Preparation:

Cholesterol Probe Warm to room temperature, store at −20° C., protect from light.

Cholesterol Esterase Dissolve in 220 μl Cholesterol Assay Buffer. Aliquot and store at −20° C.

Enzyme Mix: Dissolve in 220 μl Cholesterol Assay Buffer prior to use. Aliquot and store at −20° C.

### 3. HDL and LDL/VLDL Cholesterol Assay Protocol:

3.1. Separation of HDL and LDL/VLDL: Mix 100 μl of 2× Precipitation Buffer with 100 μl of serum sample in microcentrifuge tubes. Incubate 10 min at RT, centrifuge at 2000×g (5000 rpm) for 10 min. Transfer the supernatant (HDL) into new labeled tubes. Spin the precipitates (LDL/VLDL) again, Remove HDL supernatant. Resuspend the precipitate in 200 μl PBS.

Note A: If the supernatant is cloudy, the sample should be re-centrifuged. If the sample remains cloudy, dilute the sample 1:1 with PBS, and repeat the separation procedure. Multiply final results by two (2) due to the dilution with the 2× Precipitation Buffer.

3.2. Standard Curve and Sample Preparations: Dilute the Cholesterol Standard to 25 ng/μl by adding 10 μl of the Cholesterol Standard to 790 μl of Cholesterol Assay Buffer, Add 0, 4, 8, 12, 16, 20 μl into a series of wells in a 96-well clear bottom black plate. Adjust volume to 50 μl/well with Cholesterol Assay Buffer to generate 0, 0.1, 0.2, 0.3, 0.4, 0.5 μg/well of the Cholesterol Standard. Use 5 μl of the HDL or LDL/VLDL fraction, adjust the total volume to 50 μl/well with Cholesterol Assay Buffer.

3.3. Reaction Mix Preparations: Mix enough reagents for the number of assays performed. For each assay, prepare a total 50 μl Reaction Mix containing:
45.6 μl Cholesterol Assay Buffer
0.4 μl Cholesterol Probe
2 μl Enzyme Mix
2 μl Cholesterol Esterase

3.4. Add 50 μl of the Reaction Mix to each well containing the Cholesterol Standard or test samples, mix well.

3.5. Incubate the reaction for 60 minutes at 37° C., protect from light. Measure fluorescence at Ex/Em 538/587 nm in ENSPIRE

3.6. Calculations: Subtract 0 standard reading from readings. Plot the standard curve. Apply the sample readings to the standard curve to determine sample cholesterol amount in the reaction well.

### Sample Cholesterol Concentrations:

C=A/V(μg/μl)

Where: A is the sample cholesterol amount from the standard curve (μg).

V is original sample volume added to the sample reaction well (μl).

### VI. Triglyceride Quantification by Fluorometric Method (TG)

Triglyceride Quantification Kit (Catalog #K622-100; 100 assays; Store at −20° C.)

### 1. Kit Contents:

| Components | K622-100 | Cap Code | Part Number |
|---|---|---|---|
| Triglyceride Assay Buffer | 25 ml | WM | K622-100-1 |
| Triglyceride Probe (lyophilized) | 1 vial | Red | K622-100-2 |
| Dimethylsulfoxide (DMSO, | 0.4 ml | Brown | K622-100-3 |
| Anhydrous) | | | |
| Lipase | 0.5 ml | Blue | K622-100-4 |
| Triglyceride Enzyme Mix | 1 vial | Green | K622-100-5 |
| (lyophilized) | | | |
| Triglyceride Standard (1 mM) | 0.2 ml | Yellow | K622-100-6 |

### 2. Storage and Handling:

Store kit at −20° C., protect from light. Warm Triglyceride Assay Buffer to room temperature before use. Briefly centrifuge all small vials prior to opening.

### 3. Reagents Preparation:

Triglyceride Probe: Dissolve in 220 μl anhydrous DMSO (provided) before use. Store at −20° C., protect from light and moisture.

Triglyceride Enzyme Mix: Dissolve in 220 μl Triglyceride Assay Buffer. Aliquot and store at −20° C.

Lipase: Dissolve in 220 μl Triglyceride Assay Buffer. Aliquot and store at −20° C.

### 4. Triglyceride Assay Protocol:

4.1. Standard Curve Preparation:

Re-dissolve in hot water bath (80˜100° C.) for 1 minute or until the standard looks cloudy, vortex for 30 seconds, repeat the heat and vortex one more time. Dilute the Triglyceride Standard to 0.01 mM with the Triglyceride Assay Buffer. Add 0, 10, 20, 30, 40, 50 μl into each well individually. Adjust volume to 50 μl/well with Triglyceride Assay Buffer to generate 0.1, 0.2, 0.3, 0.4, 0.5 nmol/well of Triglyceride Standard.

4.2. Sample Preparation: Add 5 μl test samples in a 96-well clear bottom black plate, Adjust to the final volume of 50 μl/well with Triglyceride Assay Buffer.

4.3. Lipase: Add 2 μl of lipase to each standard and sample well. Mix and incubate 20 min at RT to convert triglyceride to glycerol and fatty acid.

4.4. Triglyceride Reaction Mix: Mix enough reagents for the number of samples and standards to be performed: For each well, prepare a total 50 μl Reaction Mix:

| | |
|---|---|
| 47.6 μl | Triglyceride Assay Buffer |
| 0.4 μl | Triglyceride Probe |
| 2 μl | Triglyceride Enzyme Mix |

4.5. Add 50 μl of the Reaction Mix to each well containing the Triglyceride Standard, test samples and controls. Mix well. Incubate at room temperature for 30 minutes, protect from light.

4.6. Measure fluorescence at Ex/Em 535/590 nm in ENSPIRE

4.7. Calculations:

Correct background by subtracting the value derived from the 0 triglyceride standard from all sample readings. Plot the standard curve. Apply sample Readings to the standard curve.

Triglyceride Concentration can then be Calculated:

C=Ts/Sv(nmol/μl or μmol/ml or mM)

Where: Ts is triglyceride amount from standard curve (nmol).

Sv is the sample volume (before dilution) added in sample wells (μl).

### VII. Results

TABLE 12.1
Quantification of TC, HDL, LDL/VLDL and TG of sample KH 101

| | | | LDL/VLDL | |
|---|---|---|---|---|
| Sample | TC (μg/μl) | HDL (μg/μl) | (μg/μl) | TG (mmol/L) |
| KH 101 | 0.000 ± 0.000 | 0.006 ± 0.000 | 0.001± 0.000 | 0.013 ± 0.000 |

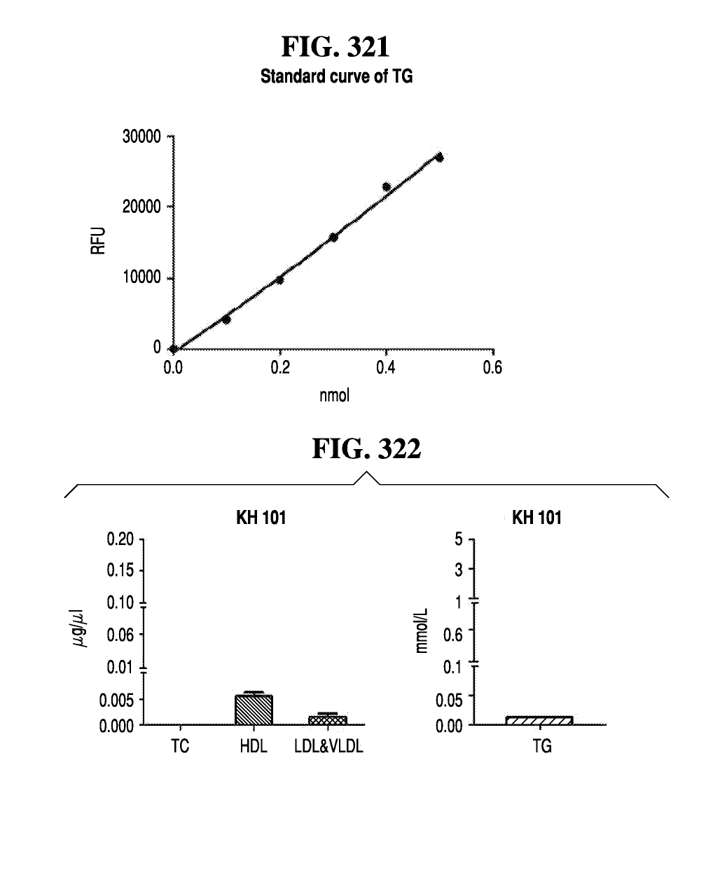

FIG. 322. Quantification of TC, HDL, LDL/VLDL and TG of sample KH 101

TABLE 12.2
Quantification of TC, HDL, LDL/VLDL and TG of sample KH102

| | | | LDL/VLDL | |
|---|---|---|---|---|
| Sample | TC (μg/μl) | HDL (μg/μl) | (μg/μl) | TG (mmol/L) |
| KH 102 | 0.002 ± 0.000 | 0.004 ± 0.000 | 0.001 ± 0.000 | 0.094 ± 0.011 |

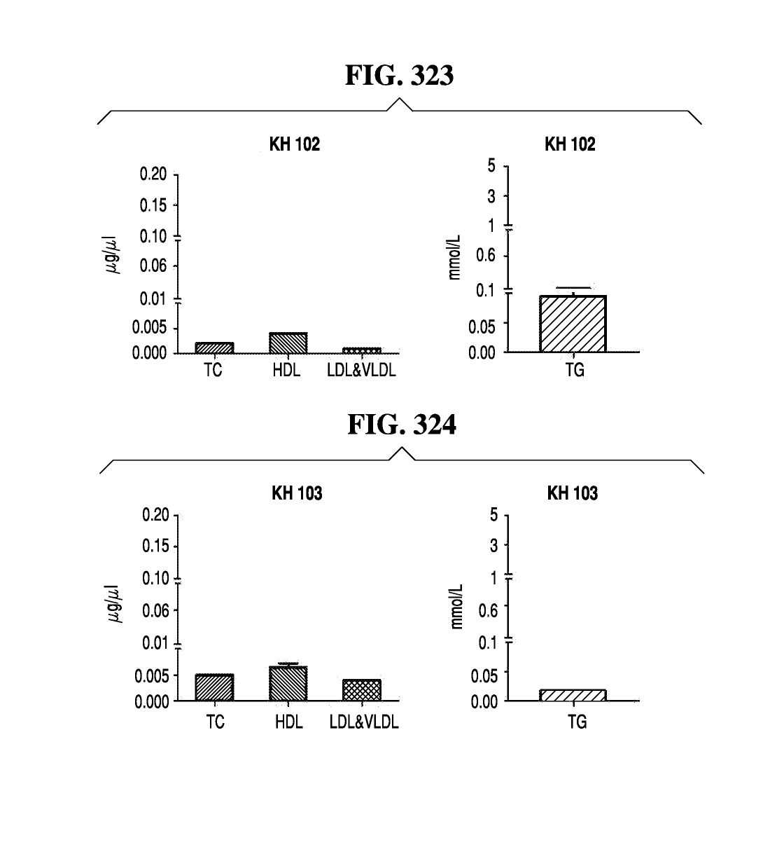

FIG. 323. Quantification of TC, HDL, LDL/VLDL and TG of sample KH 102

TABLE 12.3
Quantification of TC, HDL, LDL/VLDL and TG of sample KH 103

| | | | LDL/VLDL | |
|---|---|---|---|---|
| Sample | TC (μg/μl) | HDL (μg/μl) | (μg/μl) | TG (mmol/L) |
| KH 103 | 0.005 ± 0.000 | 0.007 ± 0.000 | 0.004 ± 0.000 | 0.018 ± 0.000 |

FIG. 324. Quantification of TC, HDL, LDL/VLDL and TG of sample KH 103

TABLE 12.4
Quantification of TC, HDL, LDL/VLDL and TG of sample KH 104

| | | | LDL/VLDL | |
|---|---|---|---|---|
| Sample | TC (μg/μl) | HDL (μg/μl) | (μg/μl) | TG (mmol/L) |
| KH 104 | 0.091 ± 0.000 | 0.052 ± | 0.003 ± 0.000 | 0.051 ± 0.006 |
| | | 0.001 | | |

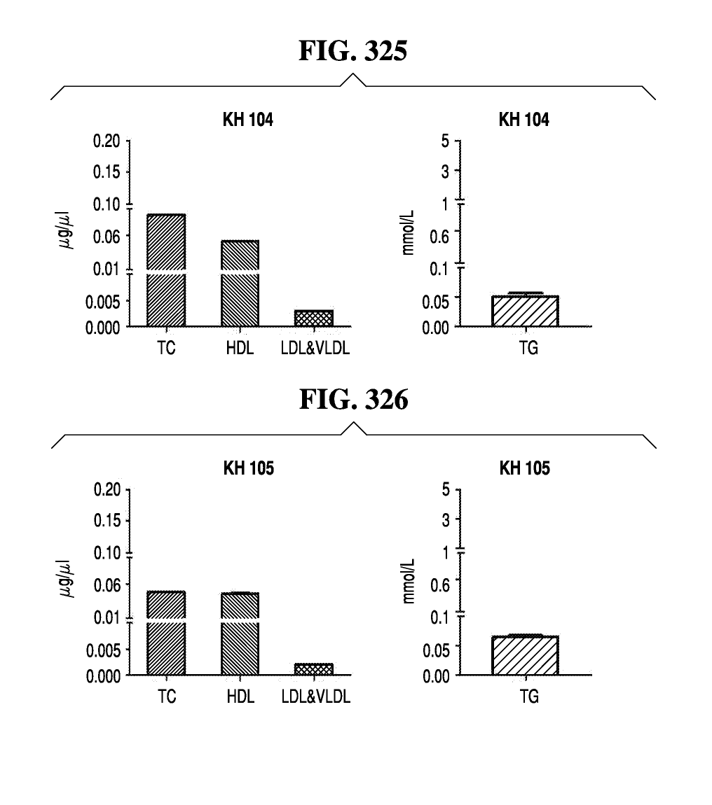

FIG. 325. Quantification of TC, HDL, LDL/VLDL and TG of sample KH 104

TABLE 12.5
Quantification of TC, HDL, LDL/VLDL and TG of sample KH 105

| | | | LDL/VLDL | |
|---|---|---|---|---|
| Sample | TC (μg/μl) | HDL (μg/μl) | (μg/μl) | TG (mmol/L) |
| KH 105 | 0.049 ± 0.001 | 0.046 ± | 0.002 ± 0.000 | 0.064 ± 0.004 |
| | | 0.001 | | |

FIG. 326. Quantification of TC, HDL, LDL/VLDL and TG of sample KH 105

TABLE 12.6
Quantification of TC, HDL, LDL/VLDL and TG of sample KH 106

| | | | LDL/VLDL | |
|---|---|---|---|---|
| Sample | TC (μg/μl) | HDL (μg/μl) | (μg/μl) | TG (mmol/L) |
| KH 106 | 0.029 ± 0.000 | 0.028 ± | 0.001 ± 0.000 | 0.021 ± 0.000 |
| | | 0.001 | | |

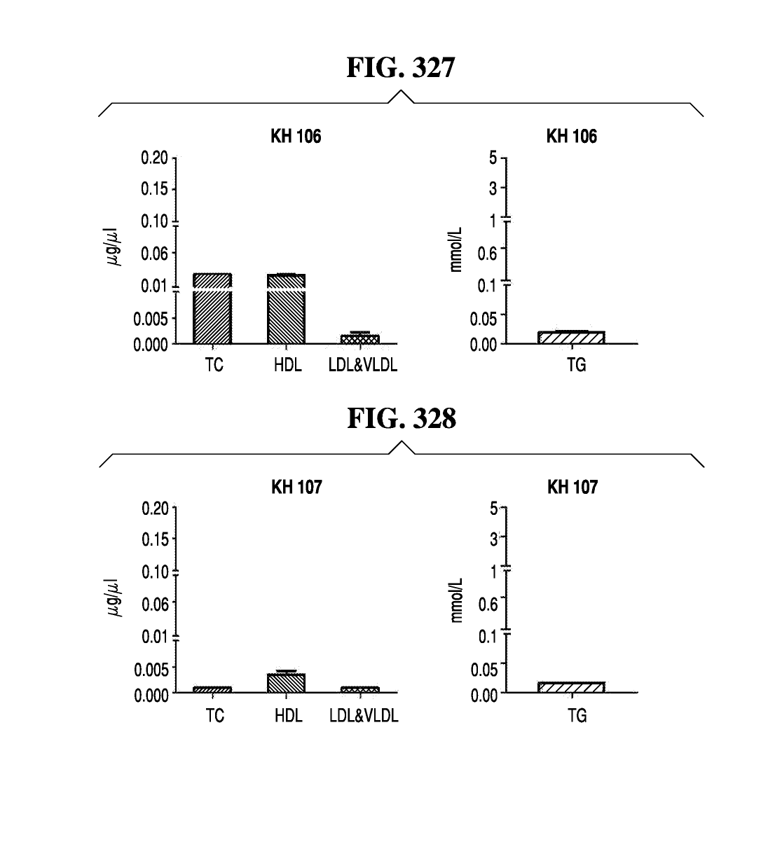

FIG. 327. Quantification of TC, HDL, LDL/VLDL and TG of sample KH106

TABLE 12.7
Quantification of TC, HDL, LDL/VLDL and TG of sample KH 107

| | | | LDL/VLDL | |
|---|---|---|---|---|
| Sample | TC (μg/μl) | HDL (μg/μl) | (μg/μl) | TG (mmol/L) |
| KH 107 | 0.001 ± 0.000 | 0.003 ± | 0.001 ± 0.000 | 0.017 ± 0.001 |
| | | 0.000 | | |

FIG. 328. Quantification of TC, HDL, LDL/VLDL and TG of sample KH 107

TABLE 12.8
Quantification of TC, HDL, LDL/VLDL and TG of sample KH 108

| | | | LDL/VLDL | |
|---|---|---|---|---|
| Sample | TC (μg/μl) | HDL (μg/μl) | (μg/μl) | TG (mmol/L) |
| KH 108 | 0.000 ± 0.000 | 0.002 ± | 0.001 ± 0.000 | 0.014 ± 0.000 |
| | | 0.000 | | |

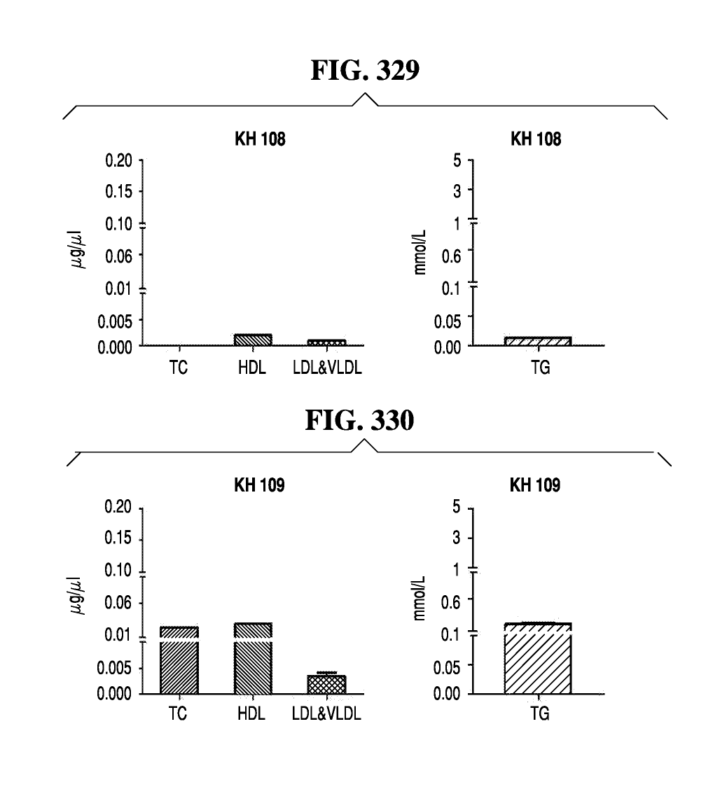

FIG. 329. Quantification of TC, HDL, LDL/VLDL and TG of sample KH 108

TABLE 12.9
Quantification of TC, HDL, LDL/VLDL and TG of sample KH 109

| | | | LDL/VLDL | |
|---|---|---|---|---|
| Sample | TC (μg/μl) | HDL (μg/μl) | (μg/μl) | TG (mmol/L) |
| KH 109 | 0.025 ± 0.001 | 0.03 ± | 0.004 ± 0.000 | 0.207 ± 0.012 |
| | | 0.000 | | |

FIG. 330. Quantification of TC, HDL, LDL/VLDL and TG of sample KH 109

TABLE 12.10
Quantification of TC, HDL, LDL/VLDL and TG of sample KH 110

| | | | LDL/VLDL | |
|---|---|---|---|---|
| Sample | TC (μg/μl) | HDL (μg/μl) | (μg/μl) | TG (mmol/L) |
| KH 110 | 0.134 ± 0.001 | 0.18 ± | 0.037 ± 0.000 | 1.684 ± 0.154 |
| | | 0.001 | | |

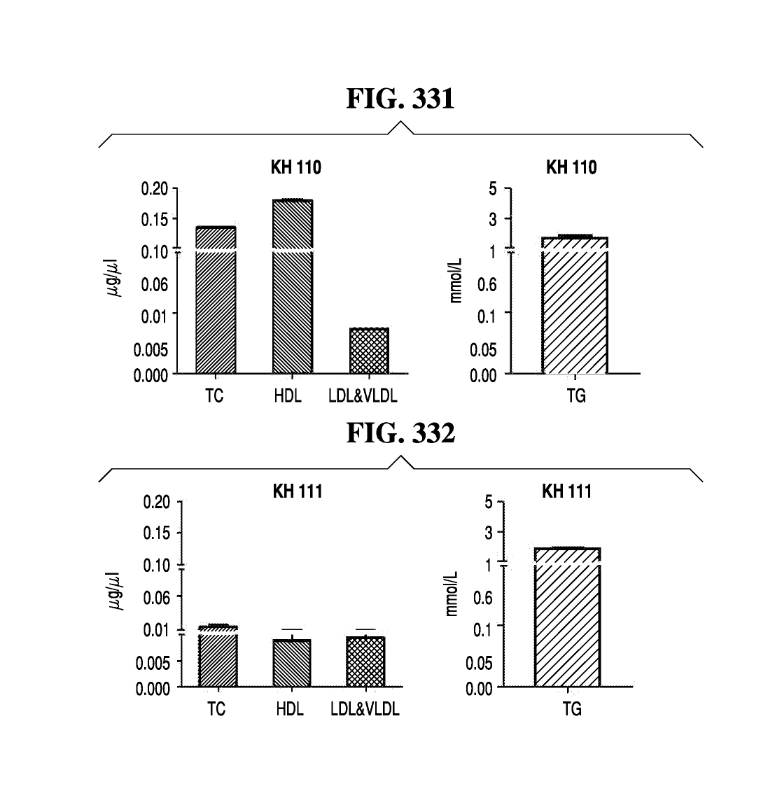

FIG. 331. Quantification of TC, HDL, LDL/VLDL and TG of sample KH 110

TABLE 12.11
Quantification of TC, HDL, LDL/VLDL and TG of sample KH 111

| | | | LDL/VLDL | |
|---|---|---|---|---|
| Sample | TC (μg/μl) | HDL (μg/μl) | (μg/μl) | TG (mmol/L) |
| KH 111 | 0.015 ± 0.003 | 0.009 ± 0.001 | 0.01 ± 0.001 | 1.865 ± 0.028 |

FIG. 332. Quantification of TC, HDL, LDL/VLDL and TG of sample KH 111

TABLE 12.12
Quantification of TC, HDL, LDL/VLDL and TG of sample KH 112

| | | | LDL/VLDL | |
|---|---|---|---|---|
| Sample | TC (μg/μl) | HDL (μg/μl) | (μg/μl) | TG (mmol/L) |
| KH 112 | 0.000 ± 0.000 | 0.006 ± | 0.003 ± 0.000 | 0.017 ± 0.004 |
| | | 0.000 | | |

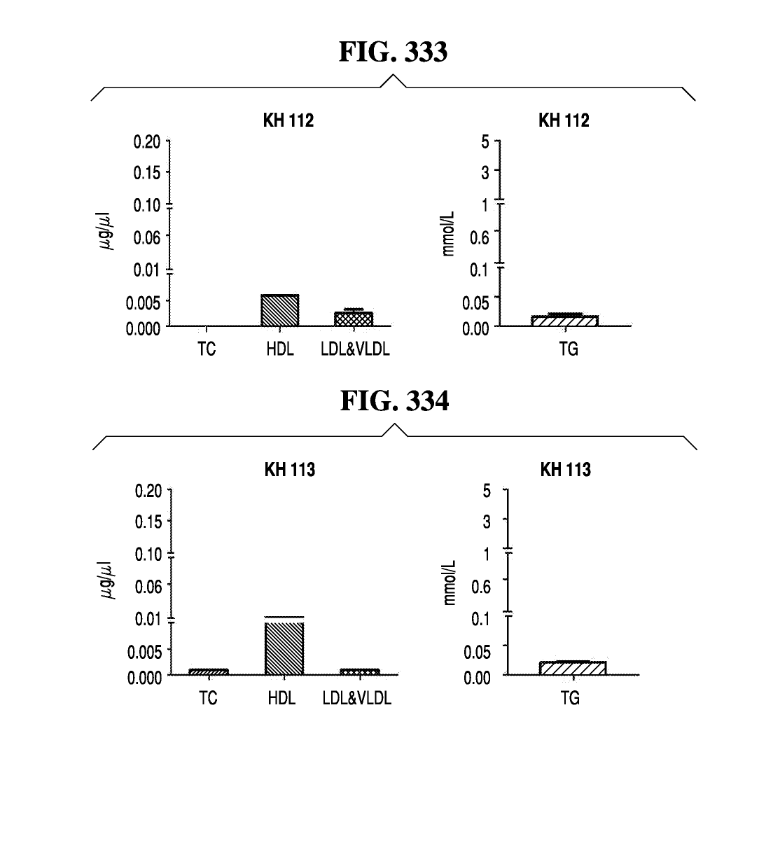

FIG. 333. Quantification of TC, HDL, LDL/VLDL and TG of sample KH 112

TABLE 12.13
Quantification of TC, HDL, LDL/VLDL and TG of sample KH 113

| | | | LDL/VLDL | |
|---|---|---|---|---|
| Sample | TC (μg/μl) | HDL (μg/μl) | (μg/μl) | TG (mmol/L) |
| KH 113 | 0.001 ± 0.000 | 0.012 ± | 0.001 ± 0.000 | 0.021 ± 0.001 |
| | | 0.000 | | |

FIG. 334. Quantification of TC, HDL, LDL/VLDL and TG of sample KH 113

TABLE 12.14
Quantification of TC, HDL, LDL/VLDL and TG of sample KH 114

| | | | LDL/VLDL | |
|---|---|---|---|---|
| Sample | TC (μg/μl) | HDL (μg/μl) | (μg/μl) | TG (mmol/L) |
| KH 114 | 0.002 ± 0.000 | 0.008 ± | 0.002 ± 0.000 | 0.232 ± 0.008 |
| | | 0.000 | | |

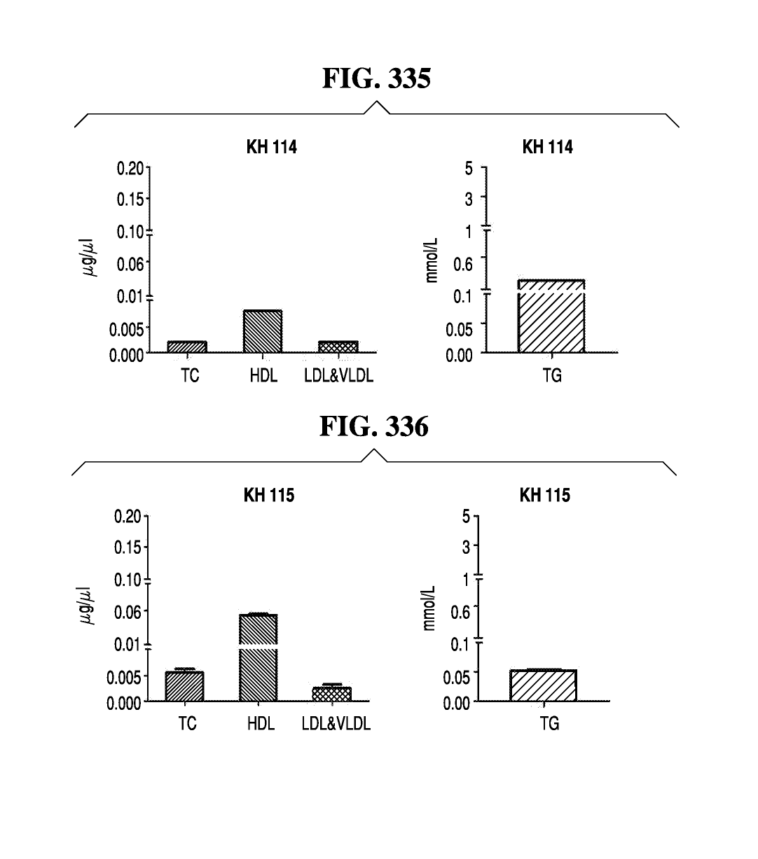

FIG. 335. Quantification of TC, HDL, LDL/VLDL and TG of sample KH 114

TABLE 12.15
Quantification of TC, HDL, LDL/VLDL and TG of sample KH 115

| | | | LDL/VLDL | |
|---|---|---|---|---|
| Sample | TC (μg/μl) | HDL (μg/μl) | (μg/μl) | TG (mmol/L) |
| KH 115 | 0.005 ± 0.001 | 0.054 ± | 0.003 ± 0.000 | 0.053 ± 0.002 |
| | | 0.002 | | |

FIG. 336. Quantification of TC, HDL, LDL/VLDL and TG of sample KH 115

TABLE 12.16
Quantification of TC, HDL, LDL/VLDL and TG of sample KH 116

| | | | LDL/VLDL | |
|---|---|---|---|---|
| Sample | TC (μg/μl) | HDL (μg/μl) | (μg/μl) | TG (mmol/L) |
| KH 116 | 0.001 ± 0.000 | 0.012 ± | 0.001 ± 0.000 | 0.027 ± 0.001 |
| | | 0.001 | | |

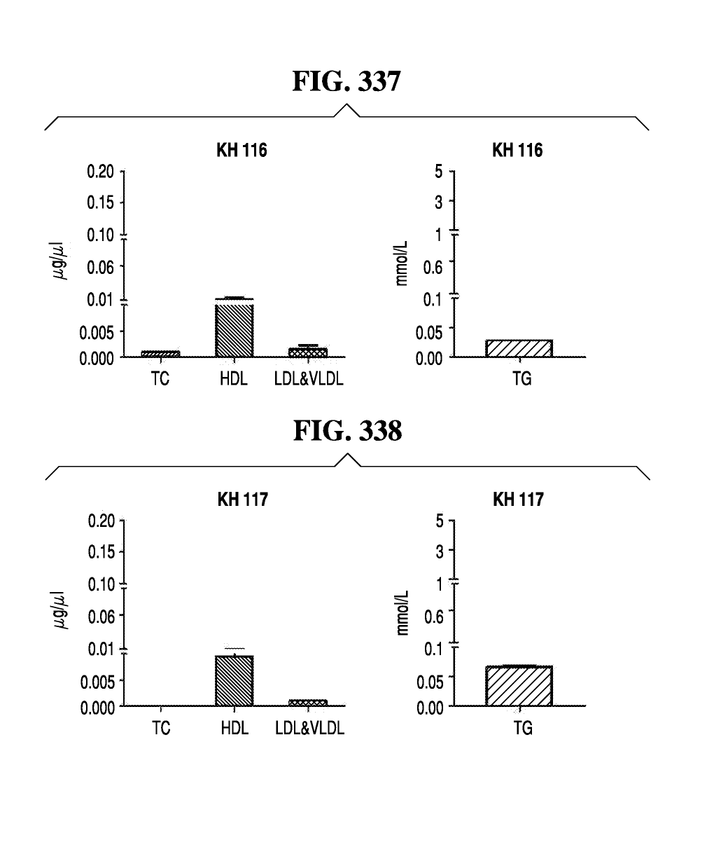

FIG. 337. Quantification of TC, HDL, LDL/VLDL and TG of sample KH 116

TABLE 12.17
Quantification of TC, HDL, LDL/VLDL and TG of sample KH 117

| | | | LDL/VLDL | |
|---|---|---|---|---|
| Sample | TC (μg/μl) | HDL (μg/μl) | (μg/μl) | TG (mmol/L) |
| KH 117 | 0.000 ± 0.000 | 0.01 ± 0.001 | 0.001 ± 0.000 | 0.066 ± 0.002 |

FIG. 338. Quantification of TC, HDL, LDL/VLDL and TG of sample KH 117

TABLE 12.18
Quantification of TC, HDL, LDL/VLDL and TG of sample KH 118

| | | | LDL/VLDL | |
|---|---|---|---|---|
| Sample | TC (μg/μl) | HDL (μg/μl) | (μg/μl) | TG (mmol/L) |
| KH 118 | 0.008 ± 0.003 | 0.031 ± | 0.004 ± 0.000 | 0.102 ± 0.001 |
| | | 0.003 | | |

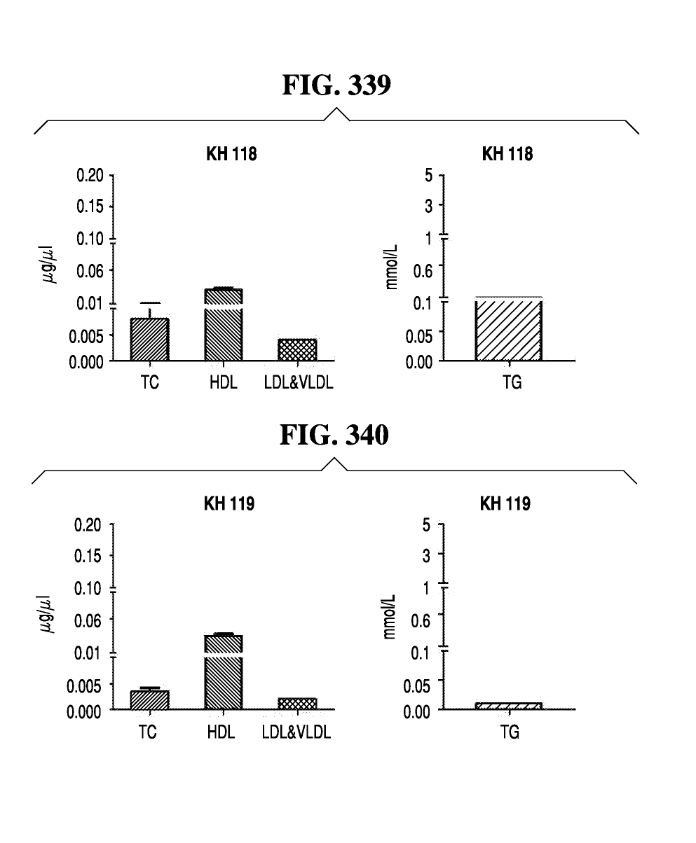

FIG. 339. Quantification of TC, HDL, LDL/VLDL and TG of sample KH 118

TABLE 12.19
Quantification of TC, HDL, LDL/VLDL and TG of sample KH 119

| Sam- | | | LDL/VLDL | |
|---|---|---|---|---|
| ple | TC (μg/μl) | HDL (μg/μl) | (μg/μl) | TG (mmol/L) |
| KH | 0.004 ± 0.000 | 0.035 ± 0.004 | 0.002 ± 0.000 | 0.011 ± 0.000 |
| 119 | | | | |

FIG. 340. Quantification of TC, HDL, LDL/VLDL and TG of sample KH 119

TABLE 12.20
Quantification of TC, HDL, LDL/VLDL and TG of sample KH 120

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.021 ± 0.001 | 0.003 ± 0.000 | 0.031 ± 0.001 |
| 120 | | | | |

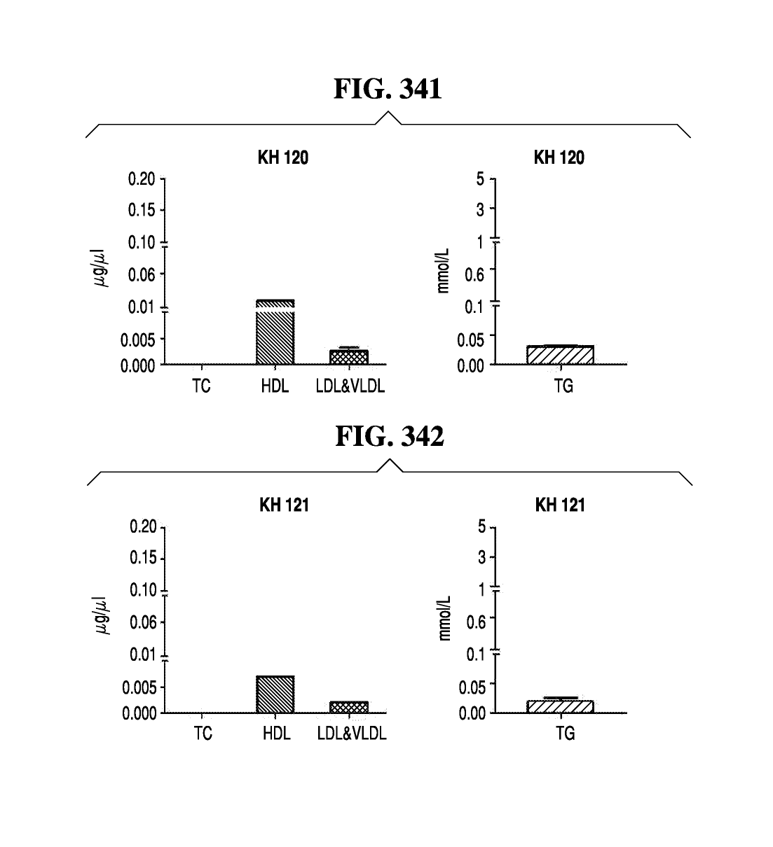

FIG. 341. Quantification of TC, HDL, LDL/VLDL and TG of sample KH 120

TABLE 12.21
Quantification of TC, HDL, LDL/VLDL and TG of sample KH 121

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.007 ± 0.000 | 0.002 ± 0.000 | 0.019 ± 0.007 |
| 121 | | | | |

FIG. 342. Quantification of TC, HDL, LDL/VLDL and TG of sample KH 121

TABLE 12.22
Quantification of TC, HDL, LDL/VLDL and TG of sample KH 122

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.008 ± 0.000 | 0.001 ± 0.000 | 0.003 ± 0.001 |
| 122 | | | | |

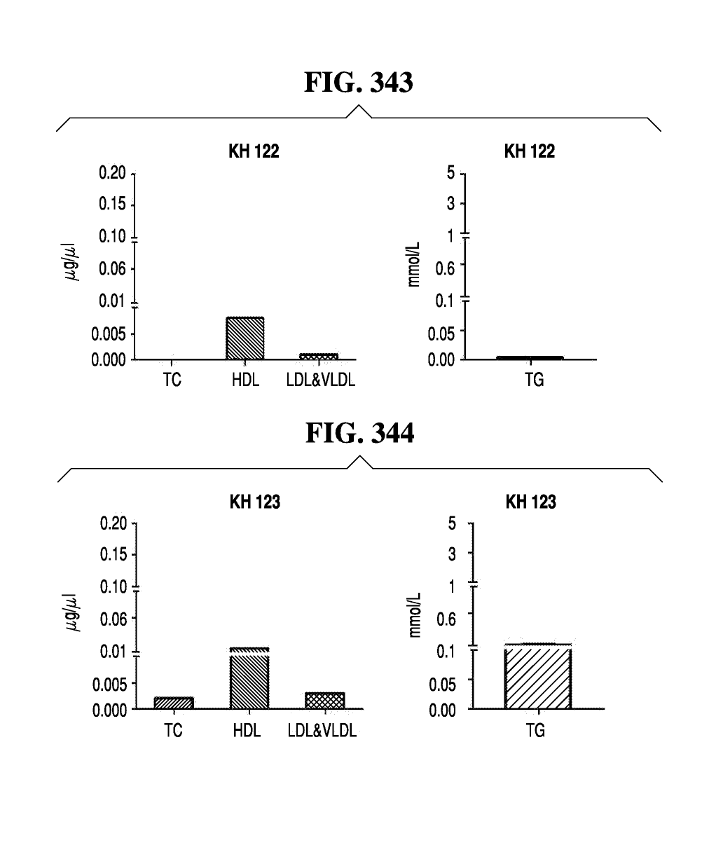

FIG. 343. Quantification of TC, HDL, LDL/VLDL and TG of sample KH 122

TABLE 12.23
Quantification of TC, HDL, LDL/VLDL and TG of sample KH 123

| Sam- | | | LDL/VLDL | |
|---|---|---|---|---|
| ple | TC (μg/μl) | HDL (μg/μl) | (μg/μl) | TG (mmol/L) |
| KH | 0.002 ± 0.000 | 0.016 ± 0.001 | 0.003 ± 0.000 | 0.104 ± 0.014 |
| 123 | | | | |

FIG. 344. Quantification of TC, HDL, LDL/VLDL and TG of sample KH 123

TABLE 12.24
Quantification of TC, HDL, LDL/VLDL and TG of sample KH 124

| Sam- | | | LDL/VLDL | |
|---|---|---|---|---|
| ple | TC (μg/μl) | HDL (μg/μl) | (μg/μl) | TG (mmol/L) |
| KH | 0.012 ± 0.000 | 0.024 ± 0.001 | 0.002 ± 0.000 | 0.015 ± 0.000 |
| 124 | | | | |

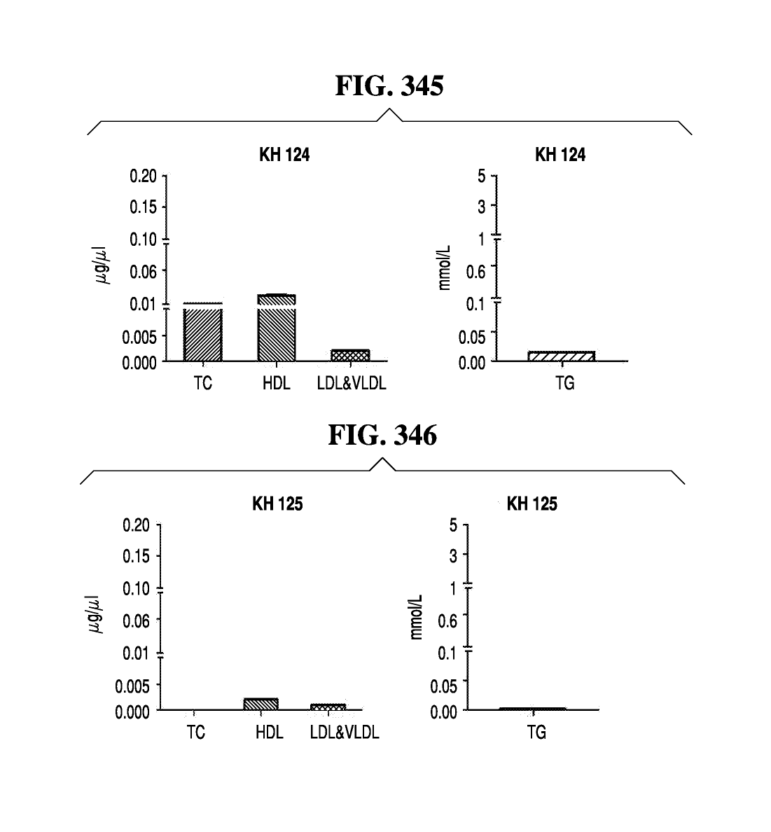

FIG. 345. Quantification of TC, HDL, LDL/VLDL and TG of sample KH 124

TABLE 12.25
Quantification of TC, HDL, LDL/VLDL and TG of sample KH 125

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.002 ± 0.000 | 0.001 ± 0.000 | 0.002 ± 0.000 |
| 125 | | | | |

FIG. 346. Quantification of TC, HDL, LDL/VLDL and TG of sample KH 125

TABLE 12.26
Quantification of TC, HDL, LDL/VLDL and TG of sample KH 126

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.002 ± 0.000 | 0.001 ± 0.000 | 0.001 ± 0.000 |
| 126 | | | | |

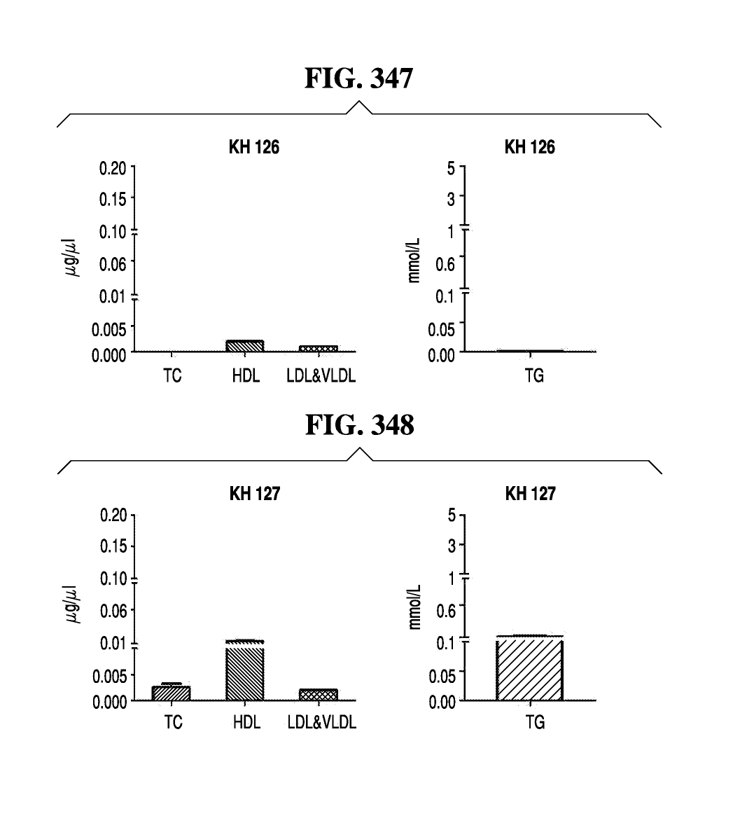

FIG. 347. Quantification of TC, HDL, LDL/VLDL and TG of sample KH 126

TABLE 12.27
Quantification of TC, HDL, LDL/VLDL and TG of sample KH 127

| Sam- | | | LDL/VLDL | |
|---|---|---|---|---|
| ple | TC (μg/μl) | HDL (μg/μl) | (μg/μl) | TG (mmol/L) |
| KH | 0.002 ± 0.000 | 0.014 ± 0.002 | 0.002 ± 0.000 | 0.113 ± 0.004 |
| 127 | | | | |

FIG. 348. Quantification of TC, HDL, LDL/VLDL and TG of sample KH 127

TABLE 12.28
Quantification of TC, HDL, LDL/VLDL and TG of sample KH 128

| Sam- | | | LDL/VLDL | |
|---|---|---|---|---|
| ple | TC (μg/μl) | HDL (μg/μl) | (μg/μl) | TG (mmol/L) |
| KH | 0.003 ± 0.000 | 0.006 ± 0.001 | 0.001 ± 0.000 | 0.014 ± 0.001 |
| 128 | | | | |

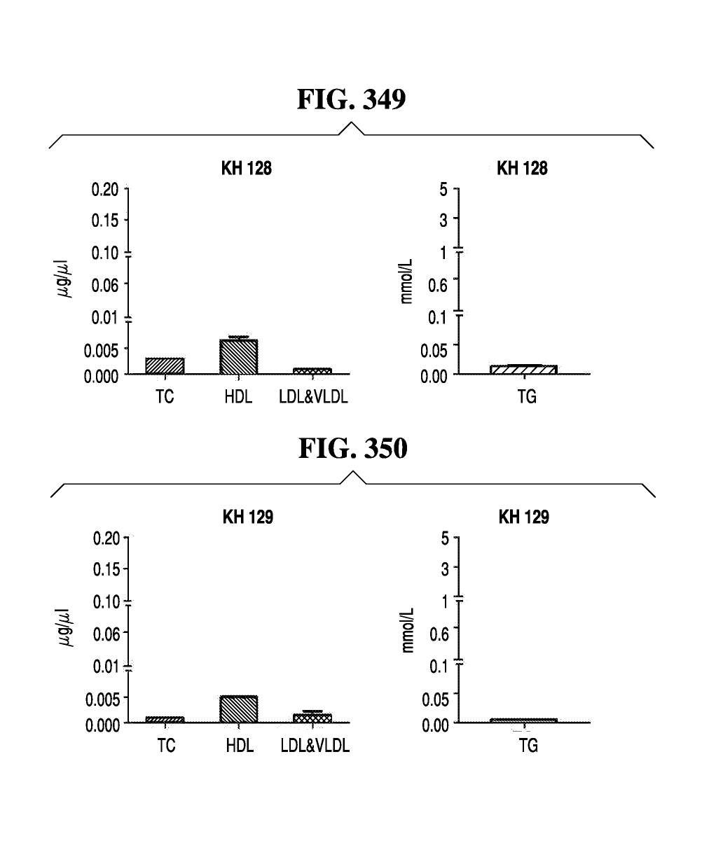

FIG. 349. Quantification of TC, HDL, LDL/VLDL and TG of sample KH 128

TABLE 12.29
Quantification of TC, HDL, LDL/VLDL and TG of sample KH 129

| Sam- | | | LDL/VLDL | |
|---|---|---|---|---|
| ple | TC (μg/μl) | HDL (μg/μl) | (μg/μl) | TG (mmol/L) |
| KH | 0.001 ± 0.000 | 0.005 ± 0.000 | 0.001 ± 0.000 | 0.006 ± 0.000 |
| 129 | | | | |

FIG. 350. Quantification of TC, HDL, LDL/VLDL and TG of sample KH 129

TABLE 12.30
Quantification of TC, HDL, LDL/VLDL and TG of sample KH 130

| Sam- | | | LDL/VLDL | |
|---|---|---|---|---|
| ple | TC (μg/μl) | HDL (μg/μl) | (μg/μl) | TG (mmol/L) |
| KH | 0.004 ± 0.000 | 0.054 ± 0.005 | 0.002 ± 0.000 | 0.015 ± 0.001 |
| 130 | | | | |

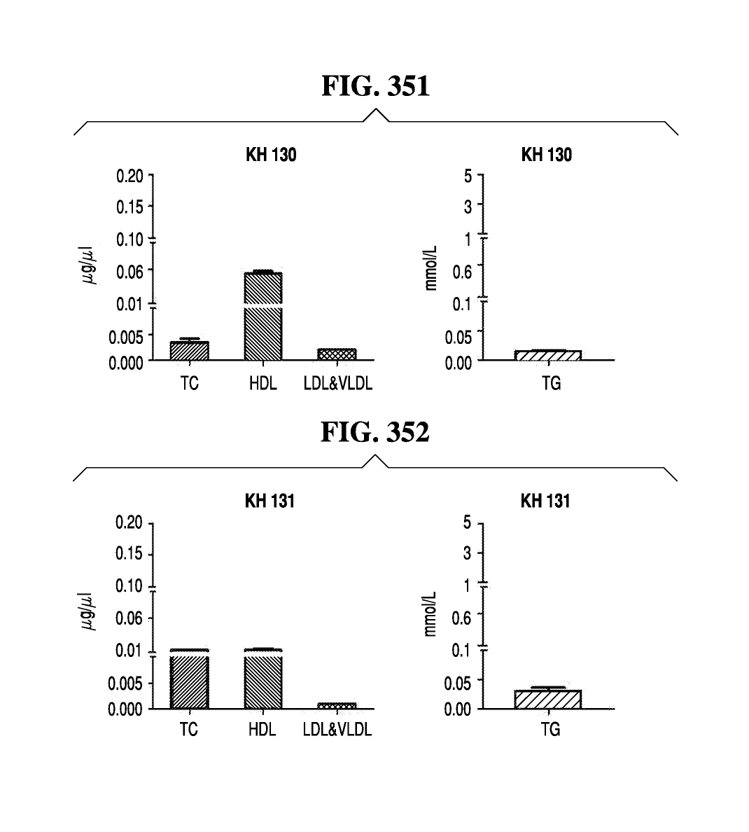

FIG. 351. Quantification of TC, HDL, LDL/VLDL and TG of sample KH 130

TABLE 12.31
Quantification of TC, HDL, LDL/VLDL and TG of sample KH 131

| Sam- | | | LDL/VLDL | |
|---|---|---|---|---|
| ple | TC (μg/μl) | HDL (μg/μl) | (μg/μl) | TG (mmol/L) |
| KH | 0.013 ± 0.001 | 0.014 ± 0.002 | 0.001 ± 0.000 | 0.031 ± 0.006 |
| 131 | | | | |

FIG. 352. Quantification of TC, HDL, LDL/VLDL and TG of sample KH 131

TABLE 12.32
Quantification of TC, HDL, LDL/VLDL and TG of sample KH 132

| Sam- | | | LDL/VLDL | |
|---|---|---|---|---|
| ple | TC (μg/μl) | HDL (μg/μl) | (μg/μl) | TG (mmol/L) |
| KH | 0.005 ± 0.000 | 0.007 ± 0.001 | 0.002 ± 0.000 | 2.928 ± 0.161 |
| 132 | | | | |

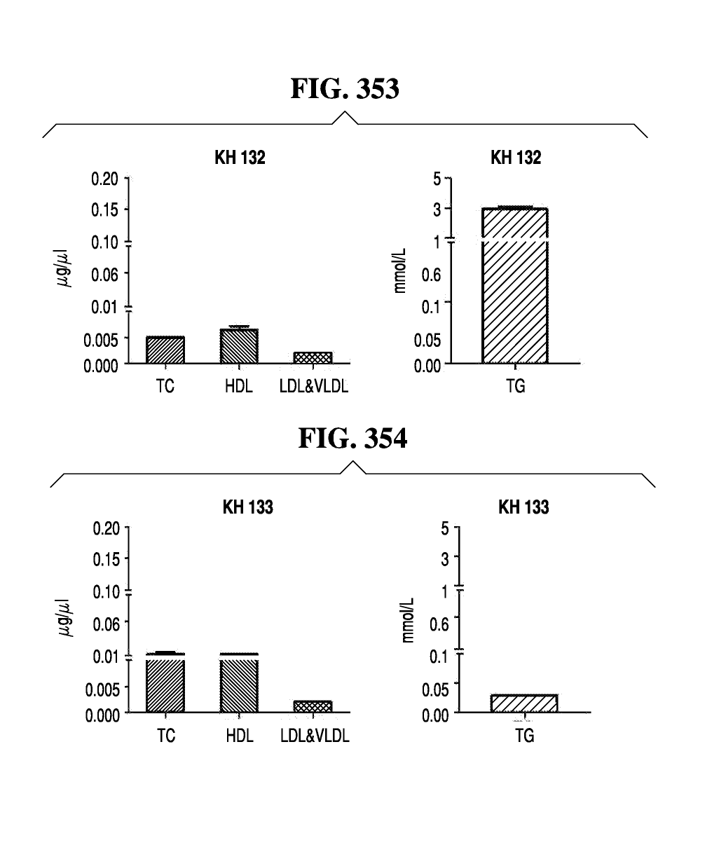

FIG. 353. Quantification of TC, HDL, LDL/VLDL and TG of sample KH 132

TABLE 12.33
Quantification of TC, HDL, LDL/VLDL and TG of sample KH 133

| Sam- | | | LDL/VLDL | |
|---|---|---|---|---|
| ple | TC (μg/μl) | HDL (μg/μl) | (μg/μl) | TG (mmol/L) |
| KH | 0.013 ± 0.003 | 0.012 ± 0.001 | 0.002 ± 0.000 | 0.029 ± 0.000 |
| 133 | | | | |

FIG. 354. Quantification of TC, HDL, LDL/VLDL and TG of sample KH 133

TABLE 12.34
Quantification of TC, HDL, LDL/VLDL and TG of sample KH 134

| Sam- | | | LDL/VLDL | |
|---|---|---|---|---|
| ple | TC (μg/μl) | HDL (μg/μl) | (μg/μl) | TG (mmol/L) |
| KH | 0.003 ± 0.000 | 0.005 ± 0.000 | 0.002 ± 0.000 | 0.054 ± 0.001 |
| 134 | | | | |

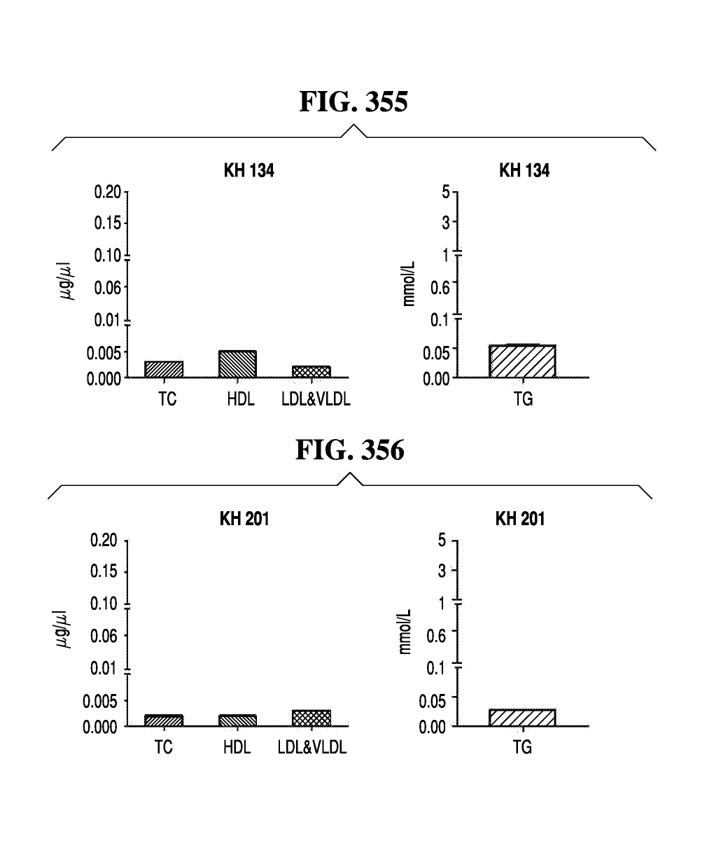

FIG. 355. Quantification of TC, HDL, LDL/VLDL and TG of sample KH 134

TABLE 12.35
Quantification of TC, HDL, LDL/VLDL and TG of sample KH 201

| Sam- | | | LDL/VLDL | |
|---|---|---|---|---|
| ple | TC (μg/μl) | HDL (μg/μl) | (μg/μl) | TG (mmol/L) |
| KH | 0.002 ± 0.000 | 0.002 ± 0.000 | 0.003 ± 0.000 | 0.027 ± 0.001 |
| 201 | | | | |

FIG. 356. Quantification of TC, HDL, LDL/VLDL and TG of sample KH 201

TABLE 12.36
Quantification of TC, HDL, LDL/VLDL and TG of sample KH 202

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.003 ± 0.000 | 0.002 ± 0.000 | 0.017 ± 0.002 |
| 202 | | | | |

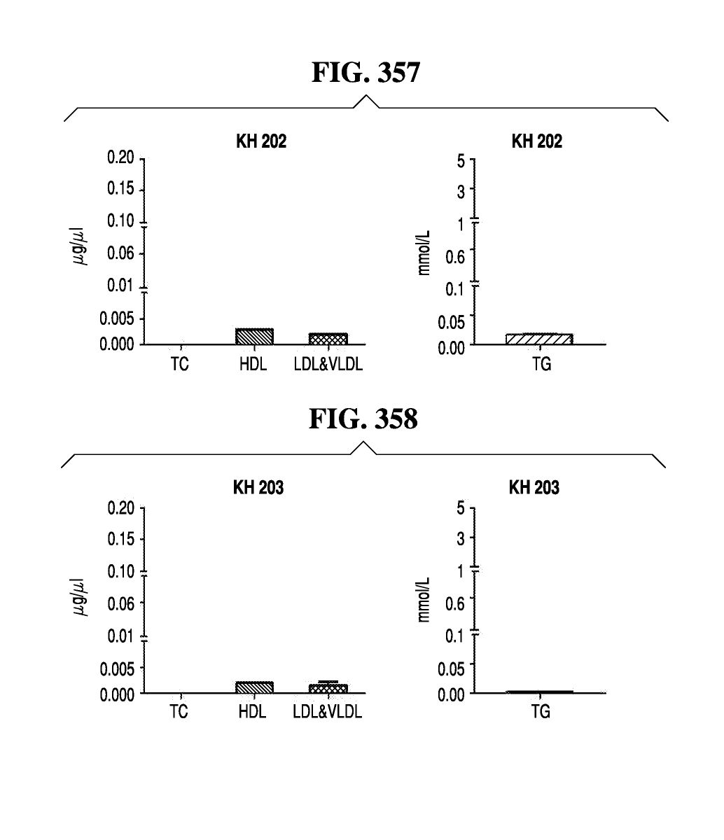

FIG. 357. Quantification of TC, HDL, LDL/VLDL and TG of sample KH 202

TABLE 12.37
Quantification of TC, HDL, LDL/VLDL and TG of sample KH 203

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.002 ± 0.000 | 0.002 ± 0.000 | 0.003 ± 0.000 |
| 203 | | | | |

FIG. 358. Quantification of TC, HDL, LDL/VLDL and TG of sample KH 203

TABLE 12.38
Quantification of TC, HDL, LDL/VLDL and TG of sample KH 204

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.003 ± 0.000 | 0.001 ± 0.000 | 0.187 ± 0.006 |
| 204 | | | | |

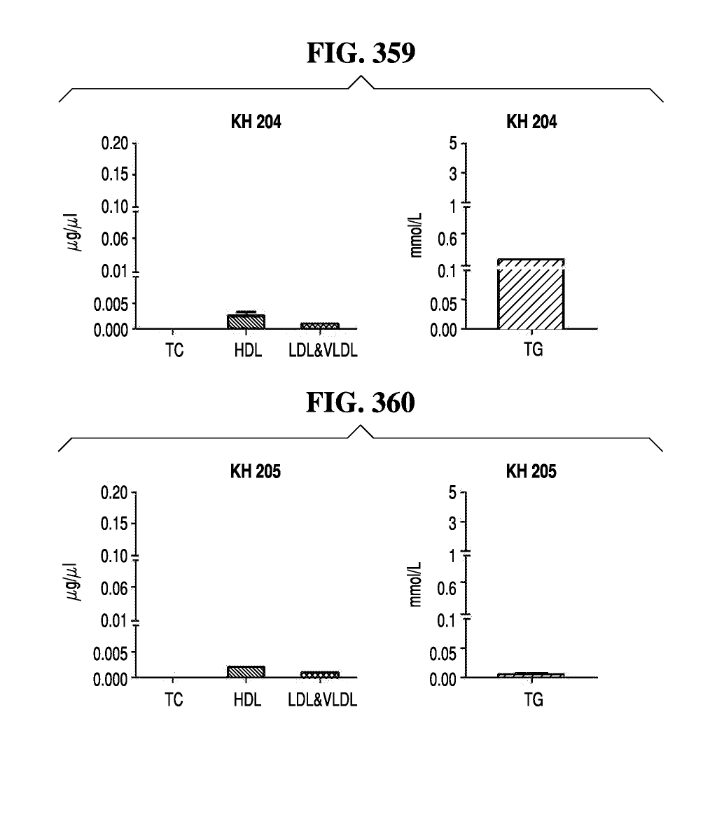

FIG. 359. Quantification of TC, HDL, LDL/VLDL and TG of sample KH 204

TABLE 12.39
Quantification of TC, HDL, LDL/VLDL and TG of sample KH 205

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.002 ± 0.000 | 0.001 ± 0.000 | 0.006 ± 0.002 |
| 205 | | | | |

FIG. 360. Quantification of TC, HDL, LDL/VLDL and TG of sample KH 205

TABLE 12.40
Quantification of TC, HDL, LDL/VLDL and TG of sample KH 206

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.002 ± 0.000 | 0.001 ± 0.000 | 0.007 ± 0.003 |
| 206 | | | | |

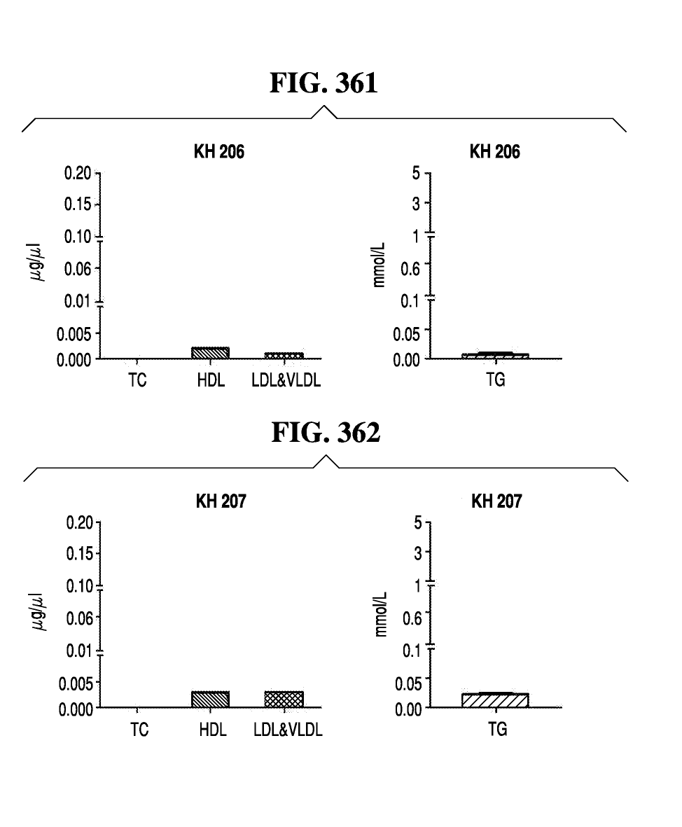

FIG. 361. Quantification of TC, HDL, LDL/VLDL and TG of sample KH 206

TABLE 12.41
Quantification of TC, HDL, LDL/VLDL and TG of sample KH 207

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.003 ± 0.000 | 0.003 ± 0.000 | 0.023 ± 0.002 |
| 207 | | | | |

FIG. 362. Quantification of TC, HDL, LDL/VLDL and TG of sample KH 207

TABLE 42
Quantification of TC, HDL, LDL/VLDL and TG of sample KH 208

| Sam- | | | LDL/VLDL | |
|---|---|---|---|---|
| ple | TC (μg/μl) | HDL (μg/μl) | (μg/μl) | TG (mmol/L) |
| KH | 0.002 ± 0.001 | 0.002 ± 0.000 | 0.002 ± 0.000 | 0.046 ± 0.005 |
| 208 | | | | |

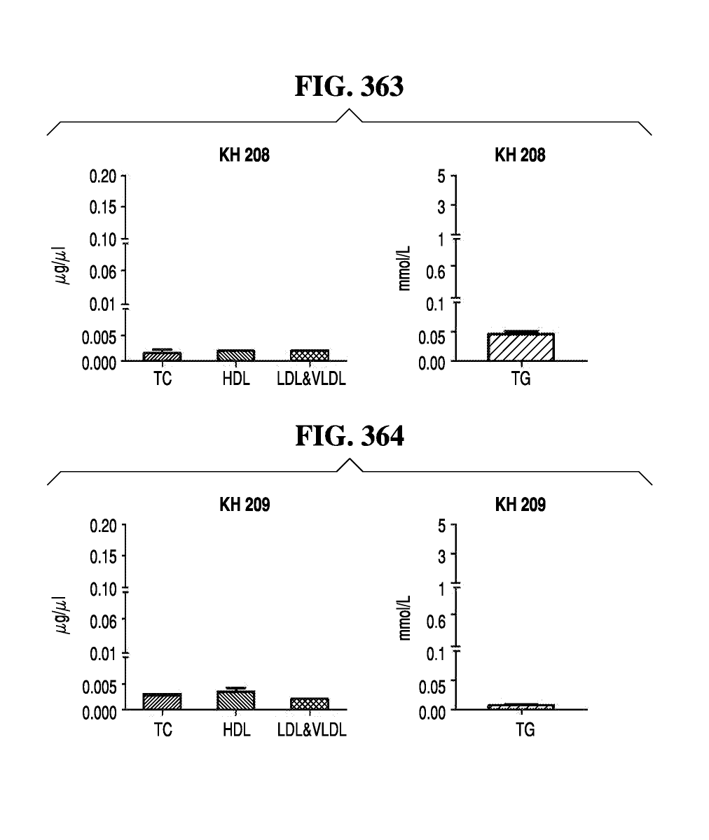

FIG. 363. Quantification of TC, HDL, LDL/VLDL and TG of sample KH 208

TABLE 12.43
Quantification of TC, HDL, LDL/VLDL and TG of sample KH 209

| Sam- | | | LDL/VLDL | |
|---|---|---|---|---|
| ple | TC (μg/μl) | HDL (μg/μl) | (μg/μl) | TG (mmol/L) |
| KH | 0.003 ± 0.000 | 0.004 ± 0.000 | 0.002 ± 0.000 | 0.007 ± 0.001 |
| 209 | | | | |

FIG. 364. Quantification of TC, HDL, LDL/VLDL and TG of sample KH 209

TABLE 12.44
Quantification of TC, HDL, LDL/VLDL and TG of sample KH 210

| Sam- | | | LDL/VLDL | |
|---|---|---|---|---|
| ple | TC (μg/μl) | HDL (μg/μl) | (μg/μl) | TG (mmol/L) |
| KH | 0.063 ± 0.001 | 0.003 ± 0.000 | 0.034 ± 0.001 | 0.752 ± 0.019 |
| 210 | | | | |

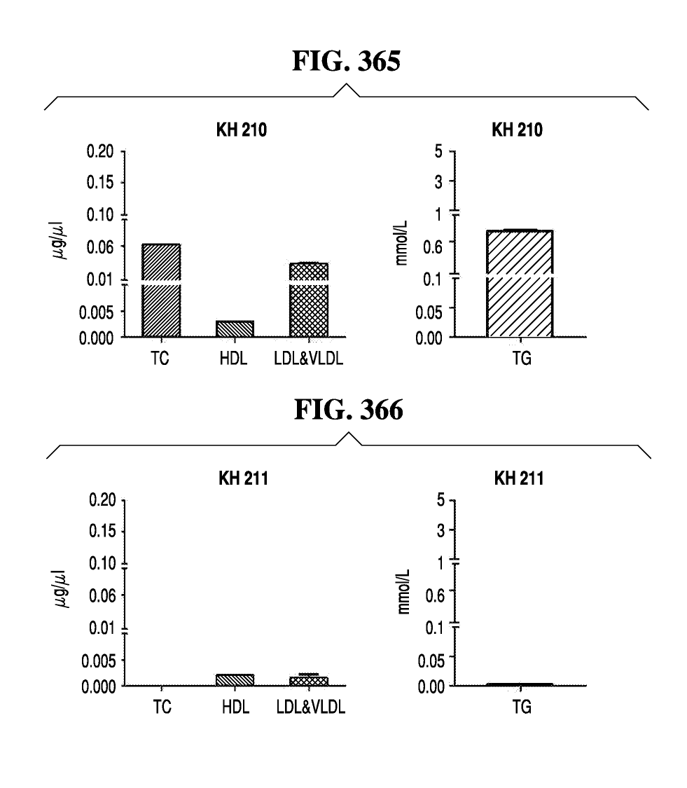

FIG. 365. Quantification of TC, HDL, LDL/VLDL and TG of sample KH 210

TABLE 12.45
Quantification of TC, HDL, LDL/VLDL and TG of sample KH 211

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.002 ± 0.000 | 0.001 ± 0.000 | 0.002 ± 0.000 |
| 211 | | | | |

FIG. 366. Quantification of TC, HDL, LDL/VLDL and TG of sample KH 211

TABLE 12.46
Quantification of TC, HDL, LDL/VLDL and TG of sample KH 212

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.002 ± 0.000 | 0.002 ± 0.000 | 0.012 ± 0.003 |
| 212 | | | | |

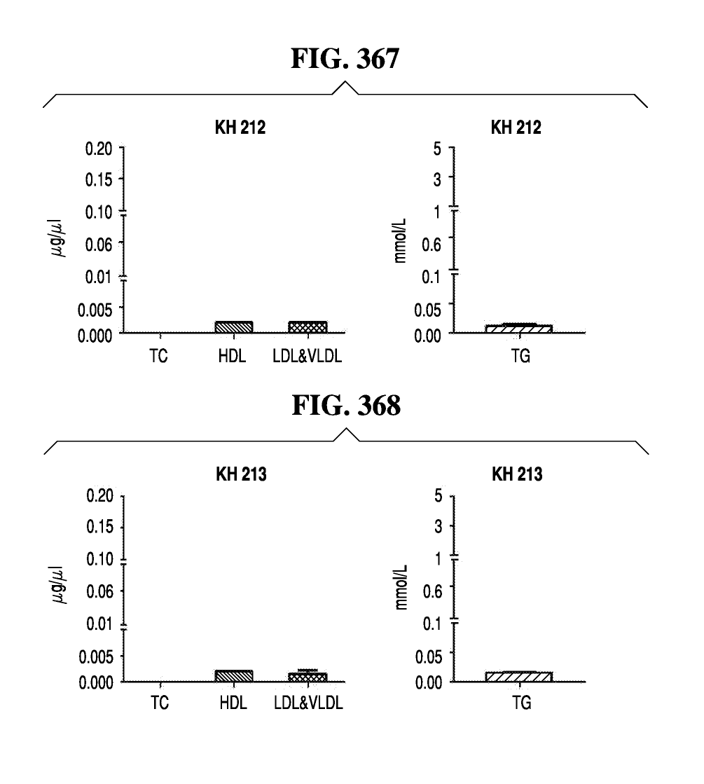

FIG. 367. Quantification of TC, HDL, LDL/VLDL and TG of sample KH 212

TABLE 12.47
Quantification of TC, HDL, LDL/VLDL and TG of sample KH 213

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.002 ± 0.000 | 0.002 ± 0.000 | 0.015 ± 0.001 |
| 213 | | | | |

FIG. 368. Quantification of TC, HDL, LDL/VLDL and TG of sample KH 213

TABLE 12.48
Quantification of TC, HDL, LDL/VLDL and TG of sample KH 214

| Sam- | | | LDL/VLDL | |
|---|---|---|---|---|
| ple | TC (μg/μl) | HDL (μg/μl) | (μg/μl) | TG (mmol/L) |
| KH | 0.004 ± 0.001 | 0.003 ± 0.000 | 0.002 ± 0.000 | 0.118 ± 0.001 |
| 214 | | | | |

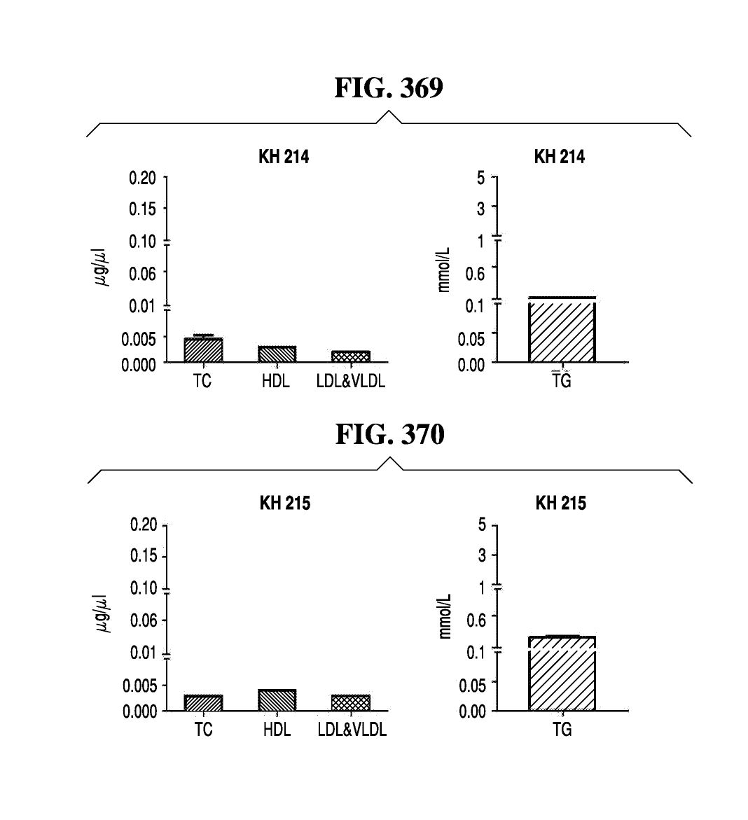

FIG. 369. Quantification of TC, HDL, LDL/VLDL and TG of sample KH 214

TABLE 12.49
Quantification of TC, HDL, LDL/VLDL and TG of sample KH 215

| Sam- | | | LDL/VLDL | |
|---|---|---|---|---|
| ple | TC (μg/μl) | HDL (μg/μl) | (μg/μl) | TG (mmol/L) |
| KH | 0.003 ± 0.000 | 0.004 ± 0.000 | 0.003 ± 0.000 | 0.258 ± 0.014 |
| 215 | | | | |

FIG. 370. Quantification of TC, HDL, LDL/VLDL and TG of sample KH 215

TABLE 12.50
Quantification of TC, HDL, LDL/VLDL and TG of sample KH 216

| Sam- | | | LDL/VLDL | |
|---|---|---|---|---|
| ple | TC (μg/μl) | HDL (μg/μl) | (μg/μl) | TG (mmol/L) |
| KH | 0.003 ± 0.000 | 0.006 ± 0.000 | 0.003 ± 0.000 | 0.318 ± 0.05 |
| 216 | | | | |

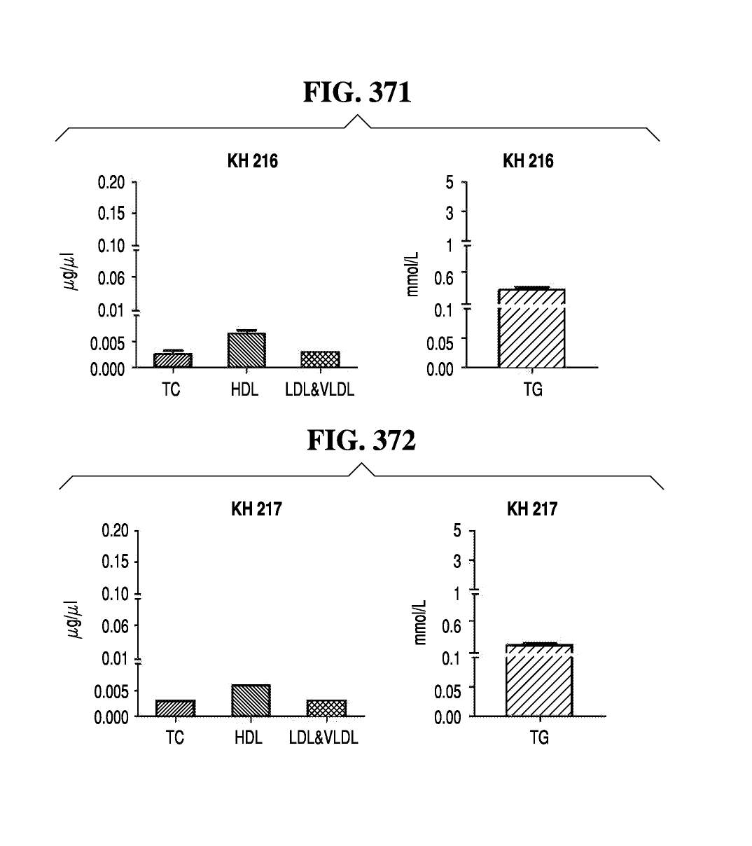

FIG. 371. Quantification of TC, HDL, LDL/VLDL and TG of sample KH 216

TABLE 12.51
Quantification of TC, HDL, LDL/VLDL and TG of sample KH 217

| Sam- | | | LDL/VLDL | |
|---|---|---|---|---|
| ple | TC (μg/μl) | HDL (μg/μl) | (μg/μl) | TG (mmol/L) |
| KH | 0.003 ± 0.000 | 0.006 ± 0.000 | 0.003 ± 0.000 | 0.223 ± 0.024 |
| 217 | | | | |

FIG. 372. Quantification of TC, HDL, LDL/VLDL and TG of sample KH 217

TABLE 12.52
Quantification of TC, HDL, LDL/VLDL and TG of sample KH 301

| Sam- | | | LDL/VLDL | |
|---|---|---|---|---|
| ple | TC (μg/μl) | HDL (μg/μl) | (μg/μl) | TG (mmol/L) |
| KH | 0.002 ± 0.000 | 0.018 ± 0.002 | 0.003 ± 0.000 | 0.079 ± 0.005 |
| 301 | | | | |

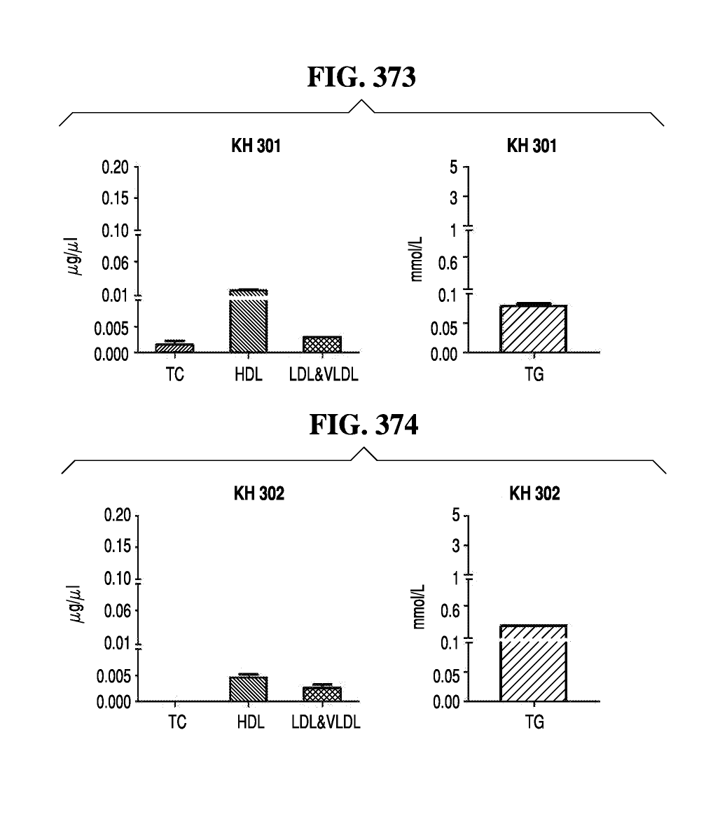

FIG. 373. Quantification of TC, HDL, LDL/VLDL and TG of sample KH 301

TABLE 12.53
Quantification of TC, HDL, LDL/VLDL and TG of sample KH 302

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.005 ± 0.000 | 0.002 ± 0.000 | 0.285 ± 0.003 |
| 302 | | | | |

FIG. 374. Quantification of TC, HDL, LDL/VLDL and TG of sample KH 302

TABLE 12.54
Quantification of TC, HDL, LDL/VLDL and TG of sample KH 303

| Sam- | | | LDL/VLDL | |
|---|---|---|---|---|
| ple | TC (μg/μl) | HDL (μg/μl) | (μg/μl) | TG (mmol/L) |
| KH | 0.000 ± 0.000 | 0.005 ± 0.001 | 0.002 ± 0.000 | 0.264 ± 0.008 |
| 303 | | | | |

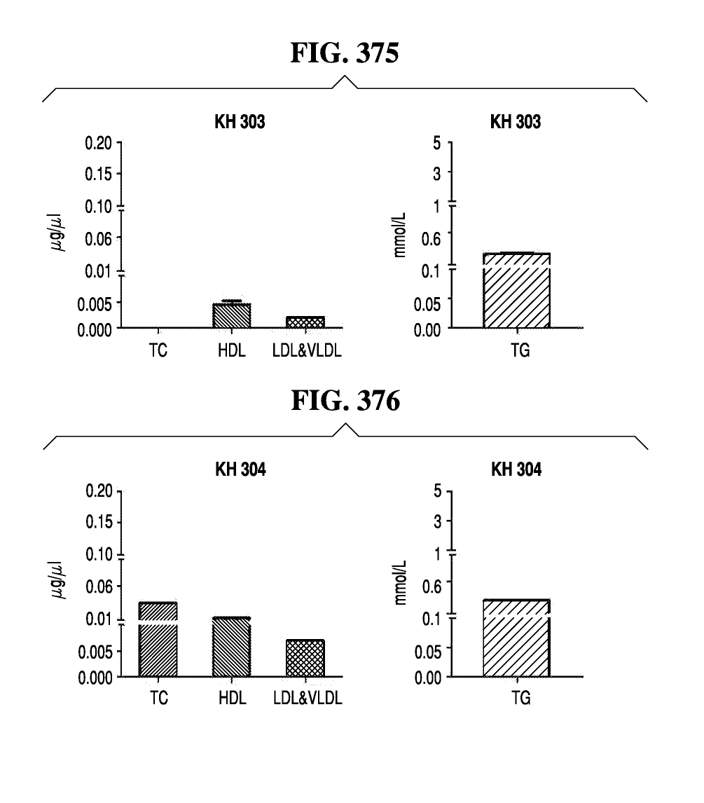

FIG. 375. Quantification of TC, HDL, LDL/VLDL and TG of sample KH 303

TABLE 12.55
Quantification of TC, HDL, LDL/VLDL and TG of sample KH 304

| Sam- | | | LDL/VLDL | |
|---|---|---|---|---|
| ple | TC (μg/μl) | HDL (μg/μl) | (μg/μl) | TG (mmol/L) |
| KH | 0.036 ± 0.001 | 0.014 ± 0.001 | 0.007 ± 0.000 | 0.301 ± 0.006 |
| 304 | | | | |

FIG. 376. Quantification of TC, HDL, LDL/VLDL and TG of sample KH 304

TABLE 12.56
Quantification of TC, HDL, LDL/VLDL and TG of sample KH 305

| Sam- | | | LDL/VLDL | |
|---|---|---|---|---|
| ple | TC (μg/μl) | HDL (μg/μl) | (μg/μl) | TG (mmol/L) |
| KH | 0.034 ± 0.001 | 0.015 ± 0.000 | 0.007 ± 0.000 | 0.302 ± 0.007 |
| 305 | | | | |

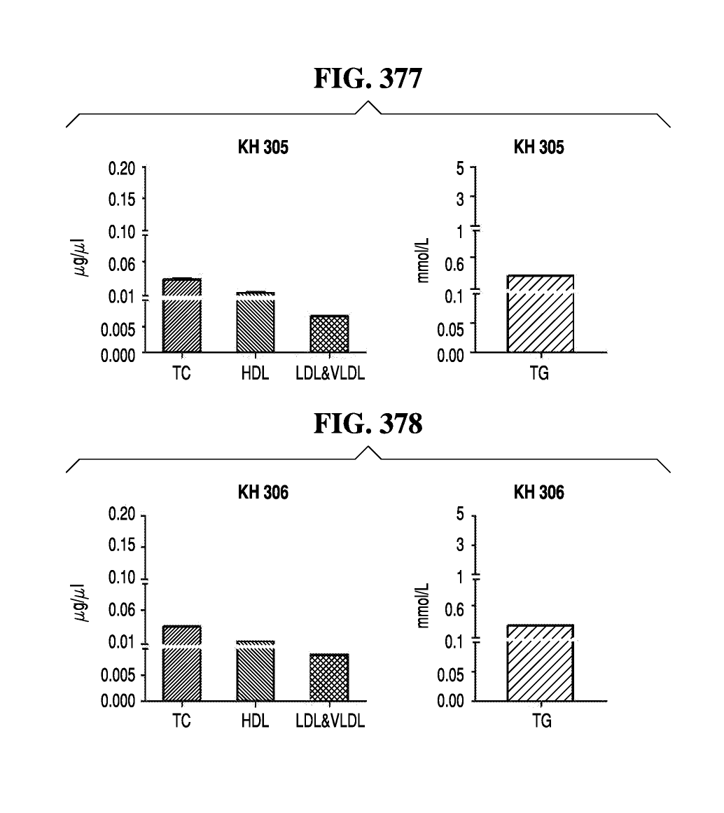

FIG. 377. Quantification of TC, HDL, LDL/VLDL and TG of sample KH 305

TABLE 12.57
Quantification of TC, HDL, LDL/VLDL and TG of sample KH 306

| Sam- | | | LDL/VLDL | |
|---|---|---|---|---|
| ple | TC (μg/μl) | HDL (μg/μl) | (μg/μl) | TG (mmol/L) |
| KH | 0.036 ± 0.001 | 0.014 ± 0.000 | 0.009 ± 0.000 | 0.297 ± 0.001 |
| 306 | | | | |

FIG. 378. Quantification of TC, HDL, LDL/VLDL and TG of sample KH 306

TABLE 12.58
Quantification of TC, HDL, LDL/VLDL and TG of sample KH 307

| Sam- | | | LDL/VLDL | |
|---|---|---|---|---|
| ple | TC (μg/μl) | HDL (μg/μl) | (μg/μl) | TG (mmol/L) |
| KH | 0.037 ± 0.002 | 0.016 ± 0.001 | 0.008 ± 0.001 | 0.296 ± 0.002 |
| 307 | | | | |

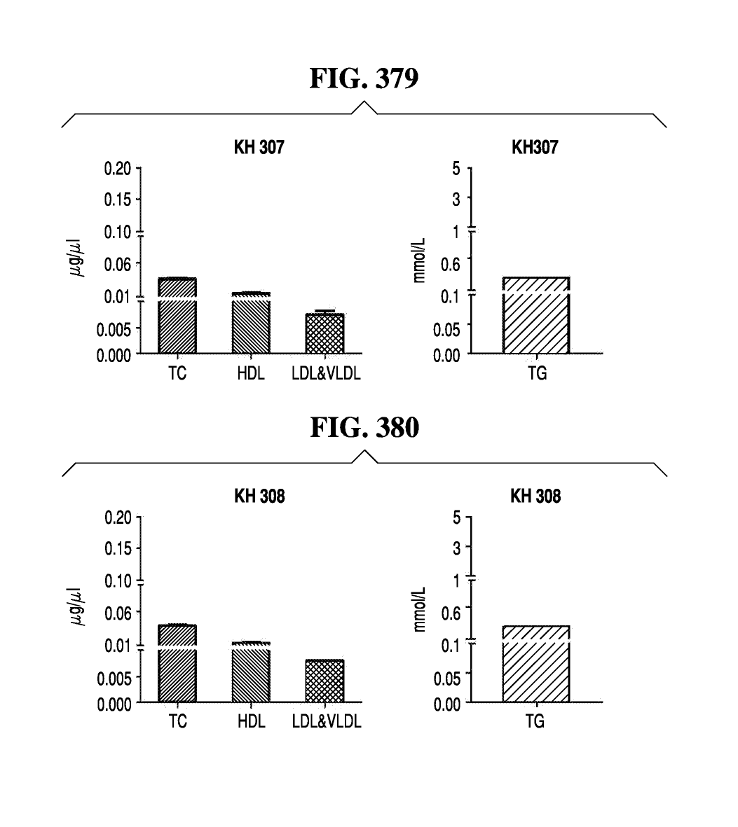

FIG. 379. Quantification of TC, HDL, LDL/VLDL and TG of sample KH 307

TABLE 12.59
Quantification of TC, HDL, LDL/VLDL and TG of sample KH 308

| Sam- | | | LDL/VLDL | |
|---|---|---|---|---|
| ple | TC (μg/μl) | HDL (μg/μl) | (μg/μl) | TG (mmol/L) |
| KH | 0.039 ± 0.001 | 0.015 ± 0.000 | 0.008 ± 0.001 | 0.289 ± 0.003 |
| 308 | | | | |

FIG. 380. Quantification of TC, HDL, LDL/VLDL and TG of sample KH 308

TABLE 12.60
Quantification of TC, HDL, LDL/VLDL and TG of sample KH 309

| | | | LDL/VLDL | |
|---|---|---|---|---|
| Sample | TC (μg/μl) | HDL (μg/μl) | (μg/μl) | TG (mmol/L) |
| KH 309 | 0.001 ± 0 | 0.004 ± 0.000 | 0.002 ± 0.000 | 0.12 ± 0.004 |

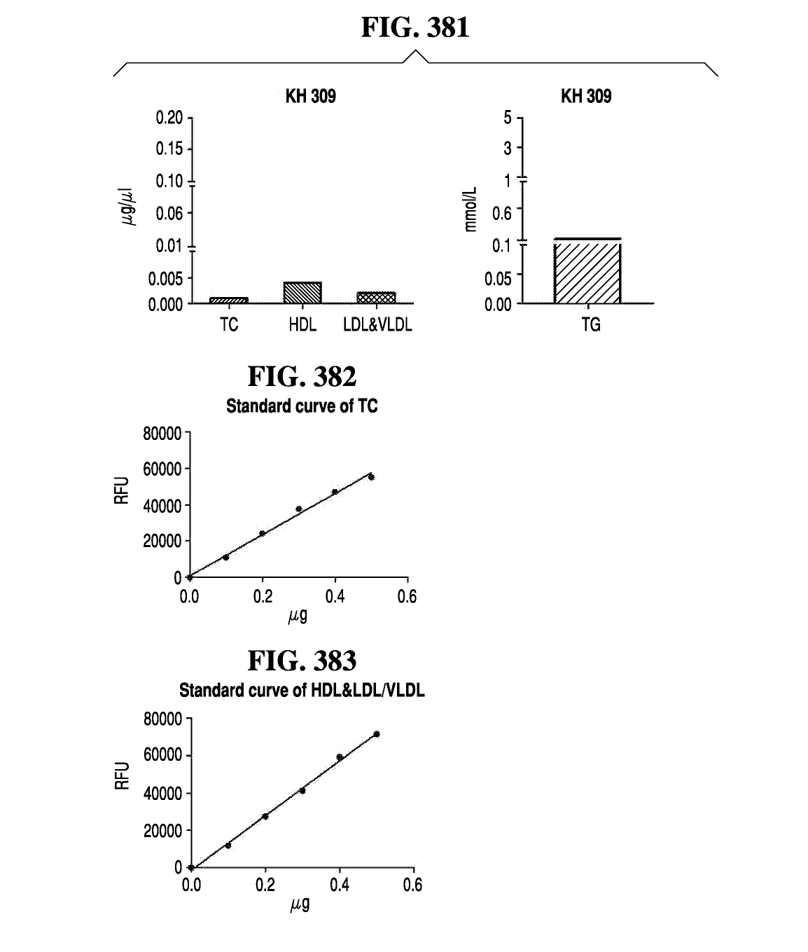

FIG. 381. Quantification of TC, HDL, LDL/VLDL and TG of sample KH 309

TABLE 12.61
Summary of TC, HDL, LDL/VLDL and TG quantification

| | TC | HDL | LDL/VLDL | TG |
|---|---|---|---|---|
| | (μg/μl) | (μg/μl) | (μg/μl) | (mmol/L) |
| KH 101 | 0.000 | 0.006 | 0.001 | 0.013 |
| KH 102 | 0.002 | 0.004 | 0.001 | 0.094 |
| KH 103 | 0.005 | 0.007 | 0.004 | 0.018 |
| KH 104 | 0.091 | 0.052 | 0.003 | 0.051 |
| KH 105 | 0.049 | 0.046 | 0.002 | 0.064 |
| KH 106 | 0.029 | 0.028 | 0.001 | 0.021 |
| KH 107 | 0.001 | 0.003 | 0.001 | 0.017 |
| KH 108 | 0.000 | 0.002 | 0.001 | 0.014 |
| KH 109 | 0.025 | 0.030 | 0.004 | 0.207 |
| KH 110 | 0.134 | 0.180 | 0.037 | 1.684 |
| KH 111 | 0.015 | 0.009 | 0.010 | 1.865 |
| KH 112 | 0.000 | 0.006 | 0.003 | 0.017 |
| KH 113 | 0.001 | 0.012 | 0.001 | 0.021 |
| KH 114 | 0.002 | 0.008 | 0.002 | 0.232 |
| KH 115 | 0.005 | 0.054 | 0.003 | 0.053 |
| KH 116 | 0.001 | 0.012 | 0.001 | 0.027 |
| KH 117 | 0.000 | 0.010 | 0.001 | 0.066 |
| KH 118 | 0.008 | 0.031 | 0.004 | 0.102 |
| KH 119 | 0.004 | 0.035 | 0.002 | 0.011 |
| KH 120 | 0.000 | 0.021 | 0.003 | 0.031 |
| KH 121 | 0.000 | 0.007 | 0.002 | 0.019 |
| KH 122 | 0.000 | 0.008 | 0.001 | 0.003 |
| KH 123 | 0.002 | 0.016 | 0.003 | 0.104 |
| KH 124 | 0.012 | 0.024 | 0.002 | 0.015 |
| KH 125 | 0.000 | 0.002 | 0.001 | 0.002 |
| KH 126 | 0.000 | 0.002 | 0.001 | 0.001 |
| KH 127 | 0.002 | 0.014 | 0.002 | 0.113 |
| KH 128 | 0.003 | 0.006 | 0.001 | 0.014 |
| KH 129 | 0.001 | 0.005 | 0.001 | 0.006 |
| KH 130 | 0.004 | 0.054 | 0.002 | 0.015 |
| KH 131 | 0.013 | 0.014 | 0.001 | 0.031 |
| KH 132 | 0.005 | 0.007 | 0.002 | 2.928 |
| KH 133 | 0.013 | 0.012 | 0.002 | 0.029 |
| KH 134 | 0.003 | 0.005 | 0.002 | 0.054 |
| KH 201 | 0.002 | 0.002 | 0.003 | 0.027 |
| KH 202 | 0.000 | 0.003 | 0.002 | 0.017 |
| KH 203 | 0.000 | 0.002 | 0.002 | 0.003 |
| KH 204 | 0.000 | 0.003 | 0.001 | 0.187 |
| KH 205 | 0.000 | 0.002 | 0.001 | 0.006 |
| KH 206 | 0.000 | 0.002 | 0.001 | 0.007 |
| KH 207 | 0.000 | 0.003 | 0.003 | 0.023 |
| KH 208 | 0.002 | 0.002 | 0.002 | 0.046 |
| KH 209 | 0.003 | 0.004 | 0.002 | 0.007 |
| KH 210 | 0.063 | 0.003 | 0.034 | 0.752 |
| KH 211 | 0.000 | 0.002 | 0.001 | 0.002 |
| KH 212 | 0.000 | 0.002 | 0.002 | 0.012 |
| KH 213 | 0.000 | 0.002 | 0.002 | 0.015 |
| KH 214 | 0.004 | 0.003 | 0.002 | 0.118 |
| KH 215 | 0.003 | 0.004 | 0.003 | 0.258 |
| KH 216 | 0.003 | 0.006 | 0.003 | 0.318 |
| KH 217 | 0.003 | 0.006 | 0.003 | 0.223 |
| KH 301 | 0.002 | 0.018 | 0.003 | 0.079 |
| KH 302 | 0.000 | 0.005 | 0.002 | 0.285 |
| KH 303 | 0.000 | 0.005 | 0.002 | 0.264 |
| KH 304 | 0.036 | 0.014 | 0.007 | 0.301 |
| KH 305 | 0.034 | 0.015 | 0.007 | 0.302 |
| KH 306 | 0.036 | 0.014 | 0.009 | 0.297 |
| KH 307 | 0.037 | 0.016 | 0.008 | 0.296 |
| KH 308 | 0.039 | 0.015 | 0.008 | 0.289 |
| KH 309 | 0.001 | 0.004 | 0.002 | 0.120 |

### VIII. Raw Data

TABLE 12.62
Raw data of Total Cholesterol/Cholesteryl Ester Quantification (TC)

| | Ave. | | Concen- | | Ave |
|---|---|---|---|---|---|
| | Read | Diluted | tration | Concentration | CONC |
| TC | (RFU) | fold | 1 (μg/μl) | 2 (μg/μl) | (μg/μl) |
| STD 0 μg | 750 | | | | |
| STD | 11933 | | | | |
| 0.1 μg | | | | | |
| STD 0.2 μg | 24906 | | | | |
| STD 0.3 μg | 38419 | | | | |
| STD 0.4 μg | 47757 | | | | |
| STD 0.5 μg | 55798 | | | | |
| KH 101 | 1510 | 1 | 0.000 | 0.000 | 0.000 |
| KH 102 | 2796.5 | 1 | 0.002 | 0.002 | 0.002 |
| KH 103 | 4444.5 | 1 | 0.005 | 0.005 | 0.005 |
| KH 104 | 53036 | 1 | 0.091 | 0.091 | 0.091 |
| KH 105 | 29152.5 | 1 | 0.049 | 0.048 | 0.049 |
| KH 106 | 17864 | 1 | 0.029 | 0.029 | 0.029 |
| KH 107 | 2136 | 1 | 0.001 | 0.001 | 0.001 |
| KH 108 | 629 | 1 | 0.000 | 0.000 | 0.000 |
| KH 109 | 15733.5 | 1 | 0.025 | 0.024 | 0.025 |
| KH 110 | 77569 | 1 | 0.135 | 0.134 | 0.134 |
| KH 111 | 10180.5 | 1 | 0.017 | 0.013 | 0.015 |
| KH 112 | 1386 | 1 | 0.000 | 0.000 | 0.000 |
| KH 113 | 2029.5 | 1 | 0.001 | 0.001 | 0.001 |
| KH 114 | 2928.5 | 1 | 0.002 | 0.002 | 0.002 |
| KH 115 | 4696.5 | 1 | 0.006 | 0.005 | 0.005 |
| KH 116 | 2080.5 | 1 | 0.001 | 0.001 | 0.001 |
| KH 117 | 1667 | 1 | 0.000 | 0.000 | 0.000 |
| KH 118 | 6223 | 1 | 0.010 | 0.006 | 0.008 |
| KH 119 | 3615 | 1 | 0.004 | 0.003 | 0.004 |
| KH 120 | 1761.5 | 1 | 0.000 | 0.000 | 0.000 |
| KH 121 | 1440.5 | 1 | 0.000 | 0.000 | 0.000 |
| KH 122 | 1269 | 1 | 0.000 | 0.000 | 0.000 |
| KH 123 | 2536.5 | 1 | 0.002 | 0.002 | 0.002 |
| KH 124 | 8368 | 1 | 0.012 | 0.012 | 0.012 |
| KH 125 | 738 | 1 | 0.000 | 0.000 | 0.000 |
| KH 126 | 754 | 1 | 0.000 | 0.000 | 0.000 |
| KH 127 | 2962.5 | 1 | 0.003 | 0.002 | 0.002 |
| KH 128 | 3160.5 | 1 | 0.003 | 0.003 | 0.003 |
| KH 129 | 2309 | 1 | 0.001 | 0.001 | 0.001 |
| KH 130 | 3624 | 1 | 0.003 | 0.004 | 0.004 |
| KH 131 | 9201.5 | 1 | 0.013 | 0.014 | 0.013 |
| KH 132 | 4543.5 | 1 | 0.005 | 0.005 | 0.005 |
| KH 133 | 8953.5 | 1 | 0.011 | 0.015 | 0.013 |
| KH 134 | 3203.5 | 1 | 0.003 | 0.003 | 0.003 |
| KH 201 | 2813 | 1 | 0.002 | 0.002 | 0.002 |
| KH 202 | 1359 | 1 | 0.000 | 0.000 | 0.000 |
| KH 203 | 1200.5 | 1 | 0.000 | 0.000 | 0.000 |
| KH 204 | 1556.5 | 1 | 0.000 | 0.000 | 0.000 |
| KH 205 | 981 | 1 | 0.000 | 0.000 | 0.000 |
| KH 206 | 996 | 1 | 0.000 | 0.000 | 0.000 |
| KH 207 | 1773 | 1 | 0.000 | 0.000 | 0.000 |
| KH 208 | 2558 | 1 | 0.002 | 0.001 | 0.002 |
| KH 209 | 3249 | 1 | 0.003 | 0.003 | 0.003 |
| KH 210 | 37167.5 | 1 | 0.063 | 0.062 | 0.063 |
| KH 211 | 825 | 1 | 0.000 | 0.000 | 0.000 |
| KH 212 | 1462.5 | 1 | 0.000 | 0.000 | 0.000 |
| KH 213 | 1712.5 | 1 | 0.000 | 0.000 | 0.000 |
| KH 214 | 4058.5 | 1 | 0.005 | 0.004 | 0.004 |
| KH 215 | 3439 | 1 | 0.003 | 0.003 | 0.003 |
| KH 216 | 3051 | 1 | 0.002 | 0.003 | 0.003 |
| KH 217 | 3371 | 1 | 0.003 | 0.003 | 0.003 |
| KH 301 | 2486.5 | 1 | 0.001 | 0.002 | 0.002 |
| KH 302 | 1804 | 1 | 0.000 | 0.000 | 0.000 |
| KH 303 | 1731 | 1 | 0.000 | 0.000 | 0.000 |
| KH 304 | 21963 | 1 | 0.036 | 0.035 | 0.036 |
| KH 305 | 21027.5 | 1 | 0.035 | 0.034 | 0.034 |
| KH 306 | 22136 | 1 | 0.037 | 0.036 | 0.036 |
| KH 307 | 22534 | 1 | 0.038 | 0.036 | 0.037 |
| KH 308 | 23780.5 | 1 | 0.040 | 0.038 | 0.039 |
| KH 309 | 2286 | 1 | 0.001 | 0.001 | 0.001 |

FIG. 382. Standard curve of Total Cholesterol/Cholesteryl Ester Quantification (TC)

TABLE 12.63
Raw data of HDL Quantification

| | Ave. | | Concen- | | Ave |
|---|---|---|---|---|---|
| | Read | Diluted | tration | Concentration | CONC |
| HDL | (RFU) | fold | 1 (μg/μl) | 2 (μg/μl) | (μg/μl) |
| STD 0 ug | 1946 | | | | |
| STD 0.1ug | 13757 | | | | |
| STD 0.2ug | 29148 | | | | |
| STD 0.3ug | 43081 | | | | |
| STD 0.4ug | 61121 | | | | |
| STD 0.5ug | 73362 | | | | |
| KH 101 | 4471.5 | 1 | 0.006 | 0.005 | 0.006 |
| KH 102 | 3608 | 1 | 0.004 | 0.004 | 0.004 |
| KH 103 | 5321 | 1 | 0.006 | 0.007 | 0.007 |
| KH 104 | 38451.5 | 1 | 0.052 | 0.051 | 0.052 |
| KH 105 | 34439.5 | 1 | 0.047 | 0.045 | 0.046 |
| KH 106 | 20733.5 | 1 | 0.028 | 0.027 | 0.028 |
| KH 107 | 2932.5 | 1 | 0.004 | 0.003 | 0.003 |
| KH 108 | 1587.5 | 1 | 0.002 | 0.002 | 0.002 |
| KH 109 | 22108.5 | 1 | 0.030 | 0.030 | 0.030 |
| KH 110 | 66401 | 2 | 0.181 | 0.179 | 0.180 |
| KH 111 | 6792 | 1 | 0.008 | 0.010 | 0.009 |
| KH 112 | 4510 | 1 | 0.006 | 0.006 | 0.006 |
| KH 113 | 9553 | 1 | 0.012 | 0.012 | 0.012 |
| KH 114 | 6300 | 1 | 0.008 | 0.008 | 0.008 |
| KH 115 | 39847 | 1 | 0.052 | 0.055 | 0.054 |
| KH 116 | 9188 | 1 | 0.011 | 0.013 | 0.012 |
| KH 117 | 7694.5 | 1 | 0.009 | 0.010 | 0.010 |
| KH 118 | 23431.5 | 1 | 0.033 | 0.029 | 0.031 |
| KH 119 | 25846.5 | 1 | 0.032 | 0.037 | 0.035 |
| KH 120 | 15692.5 | 1 | 0.020 | 0.021 | 0.021 |
| KH 121 | 5515 | 1 | 0.007 | 0.007 | 0.007 |
| KH 122 | 6349 | 1 | 0.008 | 0.008 | 0.008 |
| KH 123 | 12080 | 1 | 0.016 | 0.016 | 0.016 |
| KH 124 | 17878.5 | 1 | 0.025 | 0.023 | 0.024 |
| KH 125 | 2166 | 1 | 0.002 | 0.002 | 0.002 |
| KH 126 | 1931.5 | 1 | 0.002 | 0.002 | 0.002 |
| KH 127 | 10559.5 | 1 | 0.013 | 0.015 | 0.014 |
| KH 128 | 5149 | 1 | 0.006 | 0.007 | 0.006 |
| KH 129 | 4295.5 | 1 | 0.005 | 0.005 | 0.005 |
| KH 130 | 39819 | 1 | 0.051 | 0.057 | 0.054 |
| KH 131 | 10398 | 1 | 0.012 | 0.015 | 0.014 |
| KH 132 | 5508 | 1 | 0.007 | 0.006 | 0.007 |
| KH 133 | 9359 | 1 | 0.013 | 0.012 | 0.012 |
| KH 134 | 4287.5 | 1 | 0.005 | 0.005 | 0.005 |
| KH 201 | 2078 | 1 | 0.002 | 0.002 | 0.002 |
| KH 202 | 2421 | 1 | 0.003 | 0.003 | 0.003 |
| KH 203 | 1861.5 | 1 | 0.002 | 0.002 | 0.002 |
| KH 204 | 2269.5 | 1 | 0.002 | 0.003 | 0.003 |
| KH 205 | 1889.5 | 1 | 0.002 | 0.002 | 0.002 |
| KH 206 | 1776 | 1 | 0.002 | 0.002 | 0.002 |
| KH 208 | 2176 | 1 | 0.002 | 0.002 | 0.002 |
| KH 209 | 3035.5 | 1 | 0.004 | 0.003 | 0.004 |
| KH 210 | 2811.5 | 1 | 0.003 | 0.003 | 0.003 |
| KH 211 | 1592 | 1 | 0.002 | 0.002 | 0.002 |
| KH 212 | 1949.5 | 1 | 0.002 | 0.002 | 0.002 |
| KH 213 | 1954 | 1 | 0.002 | 0.002 | 0.002 |
| KH 214 | 2302.5 | 1 | 0.003 | 0.003 | 0.003 |

FIG. 383. Standard curve of HDL Quantification

TABLE 12.64
Raw data of HDL Quantification

| | Ave. | | | | Ave |
|---|---|---|---|---|---|
| | Read | Diluted | Concentration | Concentration | CONC |
| HDL | (RFU) | fold | 1 (μg/μl) | 2 (μg/μl) | (μg/μl) |
| STD 0 ug | 1622 | | | | |
| STD | 12364 | | | | |
| 0.1 ug | | | | | |
| STD | 29767 | | | | |
| 0.2 ug | | | | | |
| STD | 49289 | | | | |
| 0.3 ug | | | | | |
| STD | 65566 | | | | |
| 0.4 ug | | | | | |
| STD | 76613 | | | | |
| 0.5 ug | | | | | |
| KH 207 | 1705.5 | 1 | 0.003 | 0.003 | 0.003 |
| KH 215 | 2692.5 | 1 | 0.004 | 0.004 | 0.004 |
| KH 216 | 4697 | 1 | 0.007 | 0.006 | 0.006 |
| KH 217 | 4530.5 | 1 | 0.006 | 0.006 | 0.006 |
| KH 301 | 13631 | 1 | 0.019 | 0.016 | 0.018 |
| KH 302 | 3213.5 | 1 | 0.005 | 0.004 | 0.005 |
| KH 303 | 3239.5 | 1 | 0.005 | 0.004 | 0.005 |
| KH 304 | 10368.5 | 1 | 0.014 | 0.013 | 0.014 |
| KH 305 | 11203.5 | 1 | 0.015 | 0.014 | 0.015 |
| KH 306 | 10804.5 | 1 | 0.014 | 0.014 | 0.014 |
| KH 307 | 11912 | 1 | 0.016 | 0.015 | 0.016 |
| KH 308 | 11255.5 | 1 | 0.015 | 0.014 | 0.015 |
| KH 309 | 3085.5 | 1 | 0.004 | 0.004 | 0.004 |

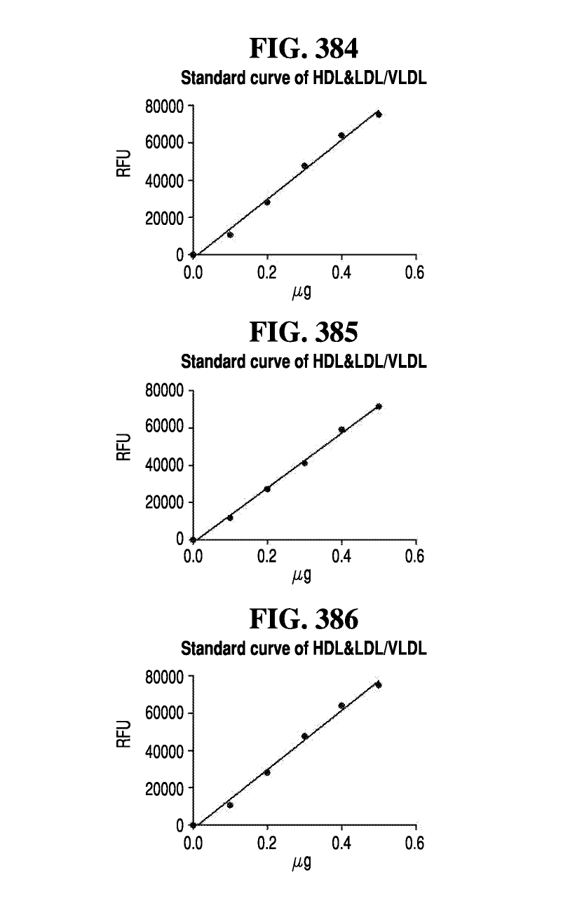

FIG. 384. Standard curve of HDL Quantification

TABLE 12.65
Raw data of LDL/VLDL Quantification

| | Ave. | | | | Ave |
|---|---|---|---|---|---|
| LDL/ | Read | Diluted | Concentration | Concentration | CONC |
| VLDL | (RFU) | fold | 1 (μg/μl) | 2 (μg/μl) | (μg/μl) |
| STD 0 ug | 1946 | | | | |
| STD | 13757 | | | | |
| 0.1 ug | | | | | |
| STD | 29148 | | | | |
| 0.2 ug | | | | | |
| STD | 43081 | | | | |
| 0.3 ug | | | | | |
| STD | 61121 | | | | |
| 0.4 ug | | | | | |
| STD | 73362 | | | | |
| 0.5 ug | | | | | |
| KH 101 | 1469.5 | 1 | 0.002 | 0.001 | 0.001 |
| KH 102 | 1130.5 | 1 | 0.001 | 0.001 | 0.001 |
| KH 103 | 3490.5 | 1 | 0.004 | 0.004 | 0.004 |
| KH 104 | 2723 | 1 | 0.003 | 0.003 | 0.003 |
| KH 105 | 2176 | 1 | 0.002 | 0.002 | 0.002 |
| KH 106 | 1444 | 1 | 0.002 | 0.001 | 0.001 |
| KH 107 | 1284.5 | 1 | 0.001 | 0.001 | 0.001 |
| KH 108 | 1198 | 1 | 0.001 | 0.001 | 0.001 |
| KH 109 | 3097.5 | 1 | 0.003 | 0.004 | 0.004 |
| KH 110 | 27844.5 | 1 | 0.037 | 0.037 | 0.037 |
| KH 111 | 7573 | 1 | 0.009 | 0.010 | 0.010 |
| KH 112 | 2270.5 | 1 | 0.002 | 0.003 | 0.003 |
| KH 113 | 1396 | 1 | 0.001 | 0.001 | 0.001 |
| KH 114 | 1794.5 | 1 | 0.002 | 0.002 | 0.002 |
| KH 115 | 2292 | 1 | 0.002 | 0.003 | 0.003 |
| KH 116 | 1463 | 1 | 0.001 | 0.002 | 0.001 |
| KH 117 | 1491.5 | 1 | 0.001 | 0.001 | 0.001 |
| KH 118 | 3526.5 | 1 | 0.004 | 0.004 | 0.004 |
| KH 119 | 1695 | 1 | 0.002 | 0.002 | 0.002 |
| KH 120 | 2315.5 | 1 | 0.003 | 0.002 | 0.003 |
| KH 121 | 1562 | 1 | 0.002 | 0.002 | 0.002 |
| KH 122 | 1344.5 | 1 | 0.001 | 0.001 | 0.001 |
| KH 123 | 2542 | 1 | 0.003 | 0.003 | 0.003 |
| KH 124 | 1810.5 | 1 | 0.002 | 0.002 | 0.002 |
| KH 125 | 1349 | 1 | 0.001 | 0.001 | 0.001 |
| KH 126 | 1257.5 | 1 | 0.001 | 0.001 | 0.001 |
| KH 127 | 1639 | 1 | 0.002 | 0.002 | 0.002 |
| KH 128 | 1420.5 | 1 | 0.001 | 0.001 | 0.001 |
| KH 129 | 1418 | 1 | 0.001 | 0.002 | 0.001 |
| KH 130 | 1969 | 1 | 0.002 | 0.002 | 0.002 |
| KH 131 | 1434.5 | 1 | 0.001 | 0.001 | 0.001 |
| KH 132 | 1597.5 | 1 | 0.002 | 0.002 | 0.002 |
| KH 133 | 1665 | 1 | 0.002 | 0.002 | 0.002 |
| KH 134 | 2097 | 1 | 0.002 | 0.002 | 0.002 |
| KH 201 | 2460.5 | 1 | 0.003 | 0.003 | 0.003 |
| KH 202 | 1683 | 1 | 0.002 | 0.002 | 0.002 |
| KH 203 | 1558 | 1 | 0.001 | 0.002 | 0.002 |
| KH 204 | 1364 | 1 | 0.001 | 0.001 | 0.001 |
| KH 205 | 1203 | 1 | 0.001 | 0.001 | 0.001 |
| KH 206 | 1401 | 1 | 0.001 | 0.001 | 0.001 |
| KH 208 | 1940 | 1 | 0.002 | 0.002 | 0.002 |
| KH 209 | 1593 | 1 | 0.002 | 0.002 | 0.002 |
| KH 210 | 25449.5 | 1 | 0.035 | 0.033 | 0.034 |
| KH 211 | 1442.5 | 1 | 0.001 | 0.002 | 0.001 |
| KH 212 | 1602.5 | 1 | 0.002 | 0.002 | 0.002 |
| KH 213 | 1544 | 1 | 0.002 | 0.001 | 0.002 |
| KH 214 | 1578 | 1 | 0.002 | 0.002 | 0.002 |

FIG. 385. Standard curve of LDL/VLDL Quantification

TABLE 12.66
Raw data of LDL/VLDL Quantification

| | Ave. | | | | Ave |
|---|---|---|---|---|---|
| LDL/ | Read | Diluted | Concentration | Concentration | CONC |
| VLDL | (RFU) | fold | 1 (μg/μl) | 2 (μg/μl) | (μg/μl) |
| STD 0 ug | 1622 | | | | |
| STD | 12364 | | | | |
| 0.1 ug | | | | | |
| STD | 29767 | | | | |
| 0.2 ug | | | | | |
| STD | 49289 | | | | |
| 0.3 ug | | | | | |
| STD | 65566 | | | | |
| 0.4 ug | | | | | |
| STD | 76613 | | | | |
| 0.5 ug | | | | | |
| KH 207 | 1632 | 1 | 0.003 | 0.003 | 0.003 |
| KH 215 | 1691 | 1 | 0.003 | 0.003 | 0.003 |
| KH 216 | 1653.5 | 1 | 0.003 | 0.003 | 0.003 |
| KH 217 | 1809 | 1 | 0.003 | 0.003 | 0.003 |
| KH 301 | 1691.5 | 1 | 0.003 | 0.003 | 0.003 |
| KH 302 | 1591 | 1 | 0.003 | 0.002 | 0.002 |
| KH 303 | 1510.5 | 1 | 0.002 | 0.002 | 0.002 |
| KH 304 | 5394.5 | 1 | 0.007 | 0.007 | 0.007 |
| KH 305 | 5359 | 1 | 0.007 | 0.007 | 0.007 |
| KH 306 | 6576.5 | 1 | 0.009 | 0.009 | 0.009 |
| KH 307 | 5754.5 | 1 | 0.008 | 0.007 | 0.008 |
| KH 308 | 5990.5 | 1 | 0.008 | 0.008 | 0.008 |
| KH 309 | 1557.5 | 1 | 0.002 | 0.002 | 0.002 |

FIG. 386. Standard curve of LDL/VLDL Quantification

TABLE 12.67
Raw data of Triglyceride Quantification (TG)

| | Ave. | | Con- | Con- | Ave |
|---|---|---|---|---|---|
| | Read | Diluted | centration | centration | CONC |
| TG | (RFU) | fold | 1 (mmol/L) | 2 (mmol/L) | (mmol/L) |
| STD | 3701 | | | | |
| 0 nmol | | | | | |
| STD | 8596 | | | | |
| 0.1 nmol | | | | | |
| STD | 16598 | | | | |
| 0.2 nmol | | | | | |
| STD | 23496 | | | | |
| 0.3 nmol | | | | | |
| STD | 29988 | | | | |
| 0.4 nmol | | | | | |
| STD | 34429 | | | | |
| 0.5 nmol | | | | | |
| KH 101 | 7629.5 | 1 | 0.013 | 0.013 | 0.013 |
| KH 102 | 33509.5 | 1 | 0.086 | 0.102 | 0.094 |
| KH 103 | 9048 | 1 | 0.018 | 0.017 | 0.018 |
| KH 104 | 19742 | 1 | 0.055 | 0.047 | 0.051 |
| KH 105 | 24052 | 1 | 0.067 | 0.062 | 0.064 |
| KH 106 | 10004 | 1 | 0.021 | 0.020 | 0.021 |
| KH 107 | 8745.5 | 1 | 0.016 | 0.017 | 0.017 |
| KH 108 | 7792.5 | 1 | 0.014 | 0.013 | 0.014 |
| KH 109 | 70010 | 1 | 0.216 | 0.199 | 0.207 |
| KH 112 | 8763 | 1 | 0.014 | 0.020 | 0.017 |
| KH 113 | 10107.5 | 1 | 0.022 | 0.020 | 0.021 |
| KH 114 | 77790 | 1 | 0.238 | 0.226 | 0.232 |
| KH 115 | 20396 | 1 | 0.054 | 0.052 | 0.053 |
| KH 116 | 12168.5 | 1 | 0.028 | 0.027 | 0.027 |
| KH 117 | 24590.5 | 1 | 0.065 | 0.067 | 0.066 |
| KH 118 | 36032 | 1 | 0.101 | 0.102 | 0.102 |
| KH 120 | 13302.5 | 1 | 0.030 | 0.032 | 0.031 |
| KH 122 | 4392 | 1 | 0.004 | 0.003 | 0.003 |
| KH 123 | 36865 | 1 | 0.114 | 0.095 | 0.104 |
| KH 124 | 8334 | 1 | 0.015 | 0.015 | 0.015 |
| KH 125 | 3996 | 1 | 0.002 | 0.002 | 0.002 |
| KH 126 | 3596.5 | 1 | 0.001 | 0.001 | 0.001 |
| KH 127 | 39651 | 1 | 0.110 | 0.116 | 0.113 |
| KH 128 | 7986.5 | 1 | 0.013 | 0.015 | 0.014 |
| KH 129 | 5412.5 | 1 | 0.006 | 0.006 | 0.006 |
| KH 130 | 8294 | 1 | 0.016 | 0.015 | 0.015 |
| KH 131 | 13312 | 1 | 0.035 | 0.027 | 0.031 |
| KH 133 | 12671.5 | 1 | 0.029 | 0.029 | 0.029 |
| KH 134 | 20743 | 1 | 0.053 | 0.055 | 0.054 |
| KH 201 | 12235 | 1 | 0.028 | 0.027 | 0.027 |
| KH 202 | 8750 | 1 | 0.015 | 0.018 | 0.017 |
| KH 203 | 4273.5 | 1 | 0.003 | 0.003 | 0.003 |
| KH 204 | 63501 | 1 | 0.192 | 0.183 | 0.187 |
| KH 205 | 5317.5 | 1 | 0.007 | 0.005 | 0.006 |
| KH 208 | 18255 | 1 | 0.050 | 0.043 | 0.046 |
| KH 209 | 5695 | 1 | 0.008 | 0.006 | 0.007 |
| KH 211 | 4056 | 1 | 0.002 | 0.002 | 0.002 |
| KH 215 | 86356 | 1 | 0.268 | 0.249 | 0.258 |
| KH 301 | 28702 | 1 | 0.075 | 0.082 | 0.079 |
| KH 302 | 94869.5 | 1 | 0.287 | 0.283 | 0.285 |
| KH 303 | 88094.5 | 1 | 0.258 | 0.269 | 0.264 |
| KH 304 | 99944 | 1 | 0.296 | 0.305 | 0.301 |
| KH 305 | 100402 | 1 | 0.297 | 0.307 | 0.302 |
| KH 306 | 98799.5 | 1 | 0.297 | 0.298 | 0.297 |
| KH 307 | 98539.5 | 1 | 0.295 | 0.298 | 0.296 |
| KH 308 | 96193.5 | 1 | 0.287 | 0.291 | 0.289 |
| KH 309 | 41917.5 | 1 | 0.123 | 0.117 | 0.120 |

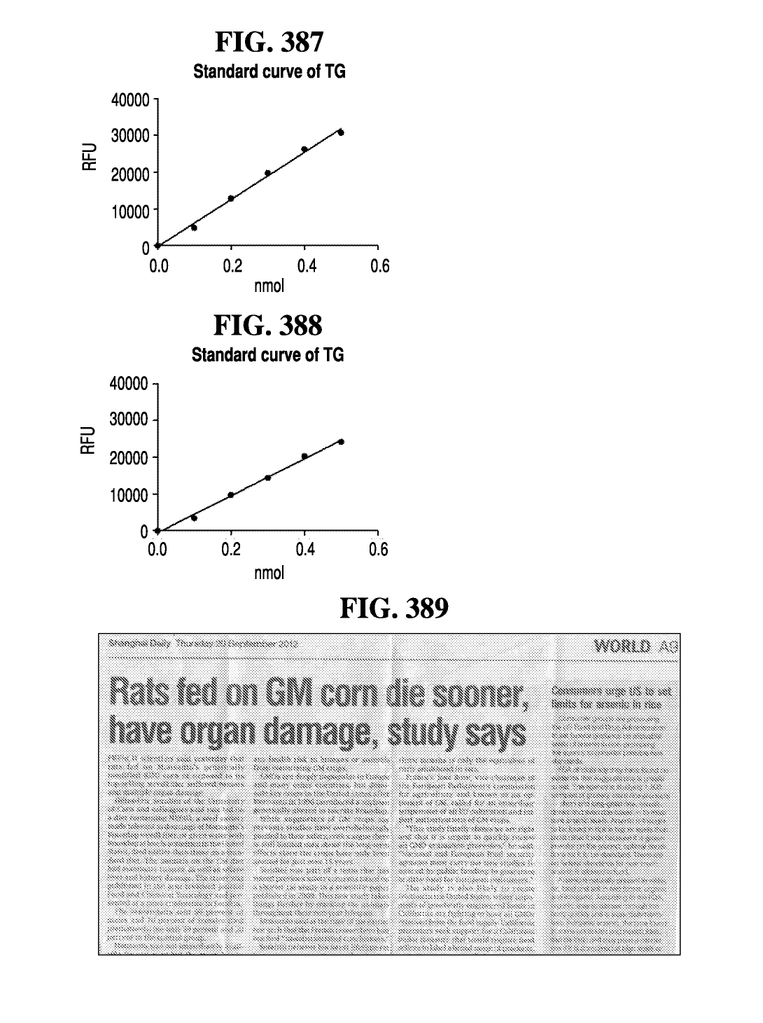

FIG. 387. Standard curve of Triglyceride Quantification (TG)

TABLE 12.68
Raw data of Triglyceride Quantification (TG)

| | Ave. | | | Con- | Ave |
|---|---|---|---|---|---|
| | Read | Diluted | Concentration | centration | CONC |
| TG | (RFU) | fold | 1 (mmol/L) | 2 (mmol/L) | (mmol/L) |
| STD | 2899 | | | | |
| 0 nmol | | | | | |
| STD | 6322 | | | | |
| 0.1 nmol | | | | | |
| STD | 12653 | | | | |
| 0.2 nmol | | | | | |
| STD | 17274 | | | | |
| 0.3 nmol | | | | | |
| STD | 23193 | | | | |
| 0.4 nmol | | | | | |
| STD | 27091 | | | | |
| 0.5 nmol | | | | | |
| KH 110 | 44712.5 | 10 | 1.793 | 1.575 | 1.684 |
| KH 111 | 49253.5 | 10 | 1.845 | 1.884 | 1.865 |
| KH 119 | 5121 | 1 | 0.011 | 0.011 | 0.011 |
| KH 121 | 7007 | 1 | 0.024 | 0.014 | 0.019 |
| KH 132 | 76021 | 10 | 3.042 | 2.814 | 2.928 |
| KH 206 | 4186.5 | 1 | 0.009 | 0.005 | 0.007 |
| KH 207 | 7996.5 | 1 | 0.024 | 0.021 | 0.023 |
| KH 210 | 21258.5 | 10 | 0.739 | 0.766 | 0.752 |
| KH 212 | 5326 | 1 | 0.014 | 0.010 | 0.012 |
| KH 213 | 6165.5 | 1 | 0.016 | 0.015 | 0.015 |
| KH 214 | 17200 | 2 | 0.118 | 0.119 | 0.118 |
| KH 216 | 10335 | 10 | 0.354 | 0.283 | 0.318 |
| KH 217 | 13552.5 | 5 | 0.206 | 0.240 | 0.223 |
| KH 302 | 23443 | 5 | 0.413 | 0.427 | 0.420 |

FIG. 388. Standard curve of Triglyceride Quantification (TG) FAT and GLUCOSE are ROTEINS, which have been proven from:

1. Plasma derived medicinal products
2. RDNA and Monoclonal products.
3. Animal protein products

### 4. Vegetables/Fruits/(Plants)

by conducting Lipid Test in a study with protocol designed by inventor and conducted at one of CRO in Shanghai.

A total of 20 different proteins from plasma derived with different concentration have been tested, all contains, TC (Total cholesterol), HDL (High Density Lipoprotein), LDL (Low Density Lipoprotein) and Triglycerides (AFOD RAAS 101-AFOD RAAS 110.

A total of 5 products from Animals (mainly Bovine and Pig) have been tested and all contains TC, HDL, LDL, and TG (P1,P2,P3,P4,P5)

A total of 6 RDNA products and Monoclonal antibodies have been tested and all contains TC, HDL, LDL, and TG (R1,R2,R3,R4,R5, and R6)

A Total of 34 Mediums From KH 101 through KH 134 except KH 102 U containing purified Urine of the Inventor and KH 108 Water for Injection from FRUITS, VEGETABLES, HOT CHILI, BLACK PEPPER have been tested and found to contain TC, HDL, LDL, and TG.

A Total of 17 Mediums from KH 201 through KH 217 from seafoods, meats, egg yoke, egg white, PORK FAT, CHICKEN FAT BEEF FAT have been tested and found to contain TC, HDL, LDL, and TG.

A total of 9 Mediums from Traditional Chinese medicine known to help stimulate sexual desire (KH 301), Very expensive chinese worm plant known to boost immune system (KH 302) and a tibet leave which is known through animal study to help insomnia mice to sleep (KH 303) Cow Milks (304-307) for baby formula and Placenta.

Due to unusual finding in the Hot pepper Chili KH 132 which has Highest Triglyceride 2.928 among all tested from KH 101 through KH 309.

Study continues on analyzing the other kind of pepper that is not very hot like Bell Pepper and Long green pepper. KH132 hot pepper chili has been known to be good for cancer but until today it has not been proven and the inventor has tested hot chili pepper vs. cancer cells and found that it inhibits the growth of the cancer cells like leukemia, breast, lung and gastric. This hot pepper remind the inventor of the case Mr. George Pino who 2000 was the general contractor to build the inventors house. The doctor had told him he had only 3 months to live as he had the gastric cancer. He still lives and continues to live until today. Six months after being told of his life expectancy by his doctor the inventor asked him how he was still alive. His claim to survival was the consumption of red hot chili peppers as he is a American/Mexican heritage. Now the inventor realize with high triglyceride will help inhibit the growth of gastric cancer.

KH 111 which contains GREEN BEAN has second highest level of Triglycerides 1.865 then KH 110, 14% RED WINE contains the third highest triglycerides with a reading of 1.684

Through this study, Inventor found that all Fruits vegetables have a level of HDL higher than LDL and unusual High Triglycerides from Hot chili (KH 132 Green Bean (KH 111) and Red Wine (KH 110).

The inventor believed that all these proteins (FAT) found in vegetables, fruits are all GOOD as they are not made from Animal and Human.

On the other hand triglycerides found in EGG YOLK (KH 210) has a reading of 0.752 and in EGG WHITE (KH 212 has a reading of 0.002 triglycerides found in PORK FAT(KH 215) has a reading of 0.258, Chicken Fat KH 216 with 0.318 and Beef Fat KH 217 with a reading of 0.223.

From 201 through 217 except KH 201 (Giant Clam) and KH 210 Egg Yolk Has a Higher reading of LDL than HDL, the rest of 15 Mediums have a reading of HDL (Good Cholesterol) HIGHER THAN LDL (Bad cholesterol)

From KH 301 through KH 309 HDL values have shown HIGHER than LDL.

Now the recent finding controversy regarding HDL and LDL and their relationship as well as TRIGLYCERIDES are being DEBATED.

In our study of number of cells in each particular medium has shown that KH 111, KH 110 KH 212, have a number of over 20,000,000 cells in 1 ML of medium whereas in KH 210 has a reading of 1,000,000 or 2,000,000 cells in 1 ML.

In a couple study for 4 weeks and 8 weeks of APOE Knock out mice had produced a very Strange result all mice in all groups show a LDL value much higher than HDL. This proves when GENE has been knocked out or transgenic mice, human or plants will have problem as the proteins in which RNA synthetize a SICK/DAMAGE/PROTEIN which feed cells causing the diseases, cancers. These have been proven for several successful animal studies for Arthritis/Parkinson diseases, Cancers, Hepatitis B, Hepatitis C and HIV viruses.

Mechanism for HUMAN, ANIMAL, PLANTS and other source are the SAME that is why:

Genetically modified rice between US government and China Government have to be suspended in Hunan province as Parents worry about EFFECTS of GM Food study on kids. Rice has been genetically modified to produce Vitamin A and a number of kids have been fed with GM Rice.

The investigation by the Ministry of Health of China is still undergoing however the inventor found that any subject from Human, Animal and plants have been genetically modified the gene will not be NORMAL as IT USED TO BE, THAT IS WHY:

RATS FED ON GM CORN DIE SOONER, HAVE ORGANS DAMAGE, Study says. (According to shanghai Daily Thursday 20 Sep. 2012)

### FIG. 389

In brief, with this invention, with new found good healthy KH proteins from Human/Animal/Plants can be used to cure for their diseases and IT IS NOT NECESSARY TO GENETICALLY MODIFIED GENE like the case of Rice, Corn, Soybean/Animal.

Any cell that have been modified will result as a bad damaged cell like the case of HEK293 which is a gene modified human cell with adenovirus 5 DNA which can produce parvovirus and lentivirus or retrovirus which are in the same family of HIV.

IN Vitro Study by CCK8 in our lab has shown that the HEK293 DOES NOT INHIBIT the growth of lung, breast, leukemia and gastric cancer cells and CHO cells.

We also found that all the cells from CHO, HEK293 and lung, breast, leukemia and gastric cancer all their cells look the SAME under optical microscope. The only difference of a good healthy KH cell and a bad, damaged, sick or cancer cell is the RNA which synthesize a good healthy protein or a bad protein which caused the disease or cancer.

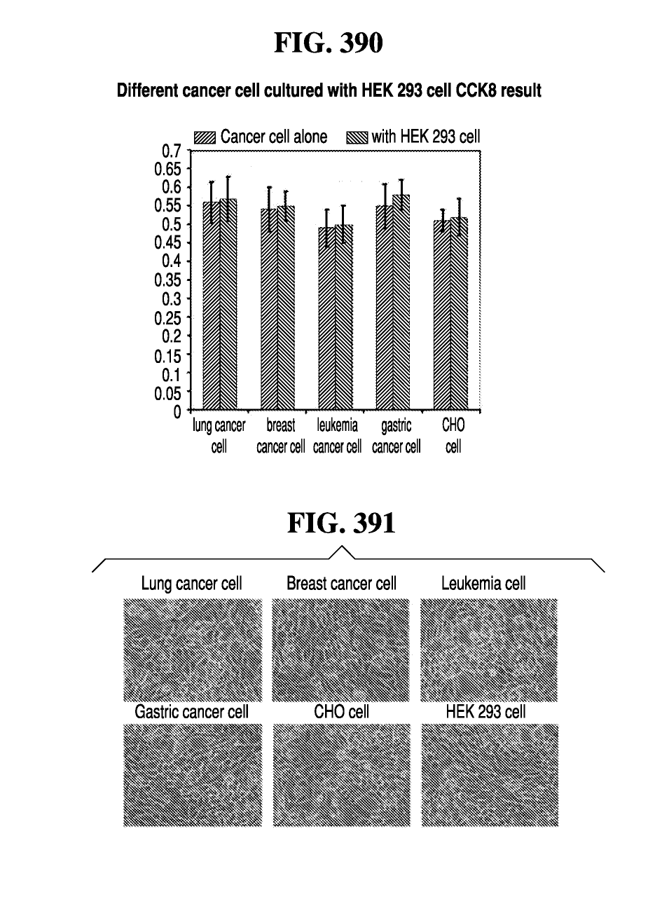

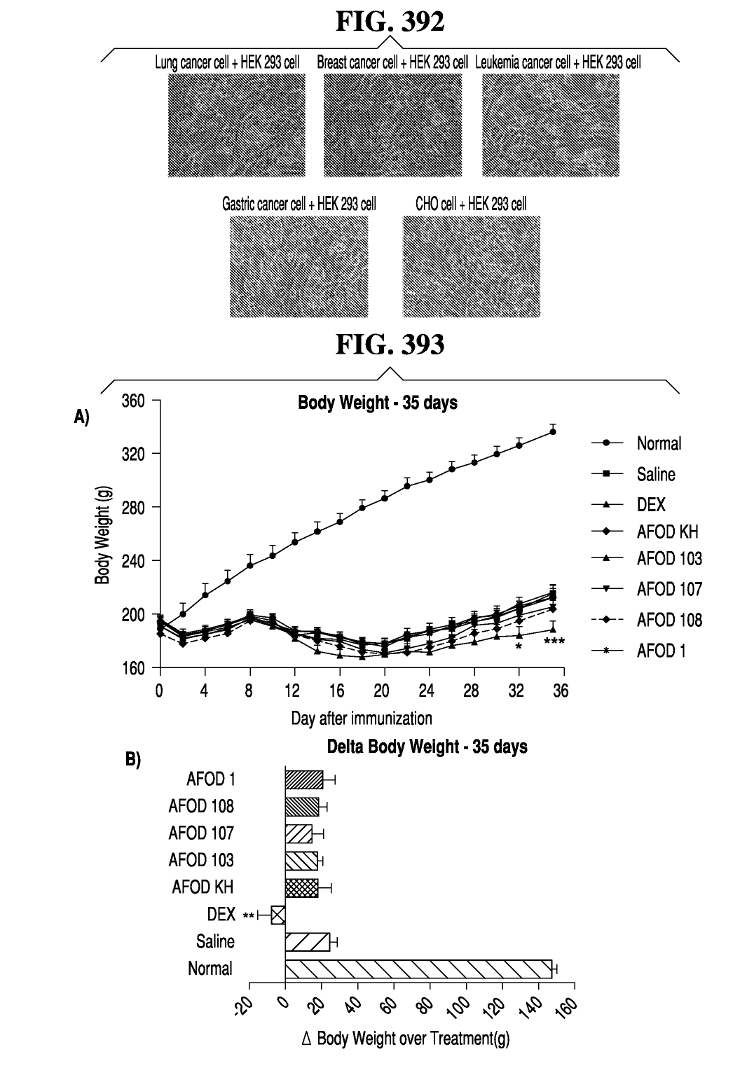

### FIG. 390; FIG. 391; FIG. 392

### Final Report

### Efficacy of Eight RAAS Test Articles on Adjuvant-Induced Arthritis (AIA) in Lewis Rats

### 1.0 Executive Summary

This study has evaluated the efficacy of eight RAAS test articles in the treatment of Adjuvant-Induced Arthritis (AIA) in Lewis rats. Male Lewis rats were immunized with Mycobacterium tuberculosis H37Ra to elicit AIA. On day 11 after immunization, when all the animals developed arthritis, the rats were administered with saline, Dexamethasone (Dex, positive control), and eight RAAS test articles for various durations, according to the sponsor's requests. The detailed treatment regimen is described below.

The data from this study showed that after the onset of the disease, the treatment with all eight RAAS products did not significantly affect the disease progression. After treatments, all the groups maintain 100% incidence rate. However, the group of animals treated with Dex had very mild disease, demonstrating dramatic inhibitory effects on the arthritic response. On the contrary, all the groups of rats treated with different RAAS products showed severe arthritis. The arthritic scores are similar among all the groups treated with RAAS products compared to that of vehicle group. Nevertheless, the measurement of paw swelling indicated that the paw volumes of the animals treated with AFCC KH and AFOD 101 decreased but the differences were not significant statistically at the most of the times compared to the vehicle group.

### 2.0 Study Personnel

3.0 The Following WuXi AppTec Personnel were Involved in the Study.

| Name | Qualifications | Title | Study Role |
|---|---|---|---|
| Shuhua Han | Ph.D. | Senior Director | Study Director |
| Jiakang Sun | Ph.D. | Group Leader | Study Monitor |
| Haiqing Chen | B.S. | Project Leader | Tester |
| Chen Fan | A.D. | Scientist | Tester |
| Hailian Xu | A.D. | Scientist | Doser |

| | |
|---|---|
| AIA | Adjuvant-induced arthritis |
| Dex | Dexamethasone |
| i.p. | intraperitoneal |
| HPMC | (Hydroxypropyl) methyl cellulose |
| p.o. | Per oral |
| b.i.d. | Twice a day |
| q.d. | Once a day |
| N/A | Not available |

### 4.0 List of Abbreviations

### 5.0 Materials and Methods

### 5.1 Experimental Groups

The original study was planned to do the treatment for 10 days after disease onset. Table 13.1 was the group setting and dosing regimen.

TABLE 13.1
Grouping and Dosing Regimen for Day 11 to 20.

| | | | | Conc. | Dose vol. | |
|---|---|---|---|---|---|---|
| Group | Test Article | N | Route | mg/ml | ml/rat | Frequency |
| 1 | Normal | 5 | N/A | N/A | N/A | N/A |
| 2 | Vehicle (Saline) | 8 | i.p. | N/A | 3 | q.d. |
| 3 | Dex ^{a} | 8 | p.o. | 0.02 | 5 ml/kg | q.d. |
| 4 | AFCC KH | 8 | i.p. | 18% | 3 | q.d. |
| 5 | AFOD KH | 8 | i.p. | 20% | 3 | q.d. |
| 6 | AFOD 101 | 8 | i.p. | 20% | 3 | q.d. |
| 7 | AFOD 102 | 8 | i.p. | 5% | 3 | q.d. |
| 8 | AFOD 103 | 8 | i.p. | 5% | 3 | q.d. |
| 9 | AFOD 107 | 8 | i.p. | 1% | 3 | q.d. |
| 10 | AFOD 108 | 8 | i.p. | 2.5% | 3 | q.d. |
| 11 | AFOD 1 | 8 | i.p. | 5% | 3 | q.d. |

^{a} 0.5% HPMC/0.02% Tween 80 made with MilliQ water as vehicle

After the completion of 10-day treatment, the sponsor requested to continue the treatment for 15 more days and to increase dosing volumes (from 3 ml/rat/day q.d., to 2.5 ml/rat/day b.i.d.) as indicated in Table 13.2.

TABLE 13.2
Grouping and Dosing Regimen for Day 21 to 35

| | | | | Conc. | Dose vol. | |
|---|---|---|---|---|---|---|
| Group | Test Article | N | Route | mg/ml | ml/rat | Frequency |
| 1 | Normal | 5 | N/A | N/A | N/A | N/A |
| 2 | Vehicle (Saline) | 8 | i.p. | N/A | 2.5 | b.i.d. |
| 3 | Dex ^{a} | 8 | p.o. | 0.02 | 5 ml/kg | q.d. |
| 4 | AFCC KH | 8 | i.p. | 18% | 2.5 | b.i.d. |
| 5 | AFOD KH | 8 | i.p. | 20% | 2.5 | b.i.d. |
| 6 | AFOD 101 | 8 | i.p. | 20% | 2.5 | b.i.d. |
| 7 | AFOD 102 | 8 | i.p. | 5% | 2.5 | b.i.d. |
| 8 | AFOD 103 | 8 | i.p. | 5% | 2.5 | b.i.d. |
| 9 | AFOD 107 | 8 | i.p. | 1-2% | 2.5 | b.i.d. |
| 10 | AFOD 108 | 8 | i.p. | 2.5% | 2.5 | b.i.d. |
| 11 | AFOD 1 | 8 | i.p. | 5% | 2.5 | b.i.d. |

^{a} 0.5% HPMC/0.02% Tween 80 made with MilliQ water as vehicle

After the completion of 25-day treatment, the sponsor requested additional 7 days treatment for five groups—Saline, Dex, AFCC KH, AFOD 101 and AFOD 102, as listed in Table 13.3. Please note that there was a two-day gap (Day 36 and 37) without treatment, before starting this 7-day period of treatment.

TABLE 13.3
Grouping and Dosing Regimen for Day 38 to Day 45:

| | | | | Conc. | Dose vol. | |
|---|---|---|---|---|---|---|
| Group | Test Article | N | Route | mg/ml | ml/rat | Frequency |
| 1 | Normal | 5 | N/A | N/A | N/A | N/A |
| 2 | Vehicle (Saline) | 8 | i.p. | N/A | 2.5 | b.i.d. |
| 3 | Dex ^{a} | 8 | p.o. | 0.02 | 5 ml/kg | q.d. |
| 4 | AFCC KH | 8 | i.p. | 18% | 2.5 | b.i.d. |
| 6 | AFOD 101 | 8 | i.p. | 20% | 2.5 | b.i.d. |
| 7 | AFOD 102 | 8 | i.p. | 28% | 2.5 | b.i.d. |

^{a} 0.5% HPMC/0.02% Tween 80 made with MilliQ water as vehicle

### 5.2 Material

### 5.2.1 Reagents

Mycobacterium tuberculosis H37Ra: Difico (Detroit, Mich., USA), Cat: 231141

Paraffin oil: China National Medicine Corporation Ltd, Cat: 30139828

Hydroxypropyl Methyl Cellulose: Sigma, Cat: C5135

Tween 80: Sigma, Sigma-Aldrich. (St. Louis, Mo., USA), Cat: P-4780

Saline: Jiangsu Kang Bao Pharmaceutical Co., Ltd. Cat: H32026295

Dexamethasone (Dex): Xinyi Pharmaceutical Co., Ltd, H31020793

5.2.2 Dose formulation and storage

All test articles were provided by the sponsor and storage at 4° C. before use.

### 5.2.3 Equipment

Plethysmometer, Italy UGO BASJLE, Biological Research Apparatus 21025

### 5.2.4 Animals and Testing Facility

| | |
|---|---|
| Species: | Rat |
| Strain: | Lewis |
| Vendor: | Beijing Vital Rivers Laboratories |
| Sex: | Male |
| Body Weight when study started | 180-200 g |
| Test Facility: | WuXi AppTec Vivarium |
| Food: | Free access to food (irradiated, |
| | Shanghai SLAC Laboratory Animal |
| | Co. Ltd., China) |
| Water: | Free access to water (municipal tap |
| | water filtered by Mol Ultrapure Water |
| | System) |
| Total number of animals | 85 |
| Animal housing: | 4 Rats/cage by treatment group |
| Identification | Each rat was identified by ear tag and |
| | cage card |
| Adaptation: | At least 7 days |
| Room: | SPF Room |
| Room temperature: | 20-26° C. |
| Room relative humidity: | 40-70% |
| Light cycle: | Fluorescent light for 12-hour light |
| | (6:00-18:00) and 12-hour dark |
| | (18:00-6:00) |
| Allocation to treatment groups: | Randomization into 11 groups to |
| | achieve similar mean body weight, |
| | minimizing bias (See Table 13.1). |

NOTE:
All of the experimental procedures carried out within this study were approved by IACUC at WuXi AppTec.

### 5.2.5 Test Article Preparation

Dex: Dex was dissolved with 0.5% HPMC/0.02% Tween 80 into a final concentration of 0.02 mg/ml. The dosing volume is 5 ml/kg. Sonicate the suspension in an ice water bath for 10 minutes. Four 12 ml aliquots were stored in 4° C. refrigerator before use.

RAAS test article: Right before each dosing, a 50 ml of aliquot of each test article was prepared and warmed to room temperature.

### 5.2.6 Immunization

### Adjuvant Preparation

Weigh 100 mg of heat-killed Mycobacterium tuberculosis, ground suspended in Paraffin oil to final concentration of 10 mg/ml.
Sonicate the suspension in an ice water bath for 15 minutes.

### Immunization Procedure

Shake the suspension of heat-killed M. tuberculosis in Paraffin oil (to ensure even distribution of bacterial particles), then draw suspension into a 1 ml glass syringe attached to a 20-G needle. Replace the needle on the glass syringe with a 25-G needle. Re-suspend material in glass syringe by rolling between hands.
Anesthetize the rats with isoflurane, then inject 0.1 ml M. tuberculosis suspension subcutaneously in the left hind foot pad.
For the normal group (n=5), mineral oil was injected subcutaneously in the left hind foot pad.
80 rats were randomly allocated to 10 groups (Table 13.1). The day of the injection was considered as day 0.

### 5.2.7 Treatment

The treatment started at Day 11 as instructed by the sponsor. The incidence rates were 100%. The original planned treatment was 10 days (Day 11 to 20), with the dosage and dosing routes indicated in Table 13.1.
Per sponsor's request, all eight test articles were continued treated for additional 15 days (Day 21 to 35), with increased dosage. The detailed dosage and regimen was listed in Table 13.2.
The sponsor requested another additional 7 days (Day 38 to 45) of treatment for Saline, Dex, AFCC KH, AFOD 101 and AFOD 102 groups (Table 13.3). There was a two days gap (Day 36 and 37) before this segment.

### 5.2.8 Endpoints

Body weight: Body weight of each animal was recorded every two days.
Paw swelling: The volume of right hind paw was pre-measured before immunization, and the right hind paw was measured once every two days, from Day 7 with plethysmometer.
Arthritic score: Start from Day 7 to 45, evaluate disease development by macroscopic inspection every two days. Assess walking ability, and screen for skin redness and swelling at the site of ankle and wrist joints and small interphalangeal joints. The left hind foot (the injected paw) will be excluded, the highest score is 12. See the criteria in table 13.4.

TABLE 13.4
Scoring system for evaluate arthritis severity

| Score | Clinical signs |
|---|---|
| 0 | No erythema or swelling |
| 1 | Slight erythema and swelling in one of the toes or fingers |
| 2 | Erythema and swelling in more than one toe or finger or mild |
| | swelling extending from the ankle to the mid-foot |
| 3 | Eryghema and severe swelling in the ankle or wrist |
| 4 | Complete erythema and swelling in toe or fingers and ankle or |
| | wrist, and inability to bend the ankle or wrist |

### 6.0 Data Analysis

Data were presented as mean±SEM. The body weight and paw volume were analyzed with two-way repeated ANOVA and the arthritis scores with Kruskal-Wallis test, by Graph Pad Prism 5. The statistical significance was noted when p<0.05.

### 7.0 Study Summary

### 7.1 Study Initiation Date and Completion Date

The study was initiated on Aug. 10, 2012, and ended on Sep. 24, 2012

### 7.2 Study Purpose

The goal of this project is to examine eight RAAS products in an autoimmune arthritis model, adjuvant induced arthritis (AIA) in rats. The study is to determine whether the products have therapeutic effects on AIA.

### 7.3 Study Results

The results of eight test articles are presented in two sections, according to their treatment durations: 1) 35 days treatment for AFOD KH, AFOD 103, AFOD 107, AFOD 108 and AFOD 1; 2) 45 days treatment for AFCC KH, AFOD 101 and AFOD 102.

### 7.3.1 Body Weight

Except Dex group, there was no significant difference for the body weight of all the treatment groups, when compared with saline group, in both 35 days and 45 days treatment sections (FIGS. 1 and 2). The reduction of body weight in Dex group was due to the side effect of Dex treatment.

FIG. 393—FIG. 1. Effects of AFOD KH, AFOD 103, AFOD 107, AFOD 108 and AFOD 1 on body weight (A) and body weight change (B) in AIA model till Day 35 (*p<0.05, **p<0.01, ***p<0.001, treatment groups v.s. saline group, two-way repeated or one-way ANOVA).

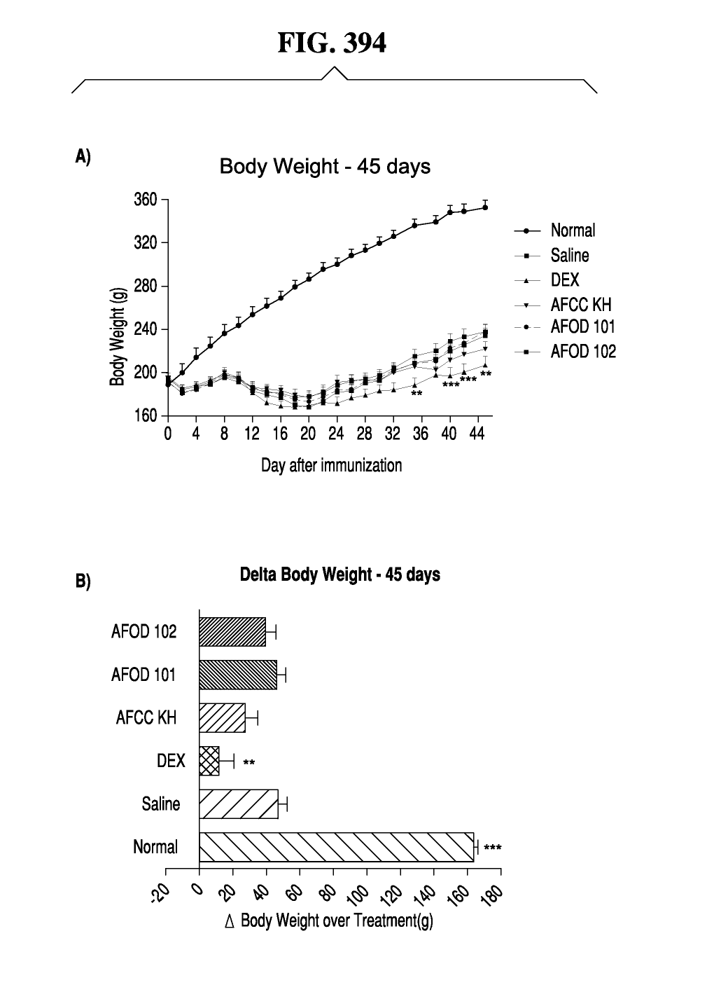

FIG. 394—Effects of AFCC KH, AFOD 101 and AFOD 102 on body weight (A) and body weight change (B) in AIA model till Day 45 (**p<0.01, ***p<0.001, treatment groups v.s. saline group, two-way repeated or one-way ANOVA).

### Paw Volume

The measurement of the paw volume indicated that the paw swelling was slightly reduced in the groups of animal treated with AFCC KH and AFOD 101. Statistical analysis showed that at the most of the times, the reduction was not significant statistically. However, the animals treated with AFCC KH showed significantly reduced paw volume on Day 22 and 35, compared to that of saline group (FIG. 4A). The animals treated with AFOD 101 showed significantly reduced paw swelling on day 22 (FIG. 4A). All other groups treated with the other six RAAS products didn't show any significant reduction in the paw swelling (FIGS. 3B & 4B).

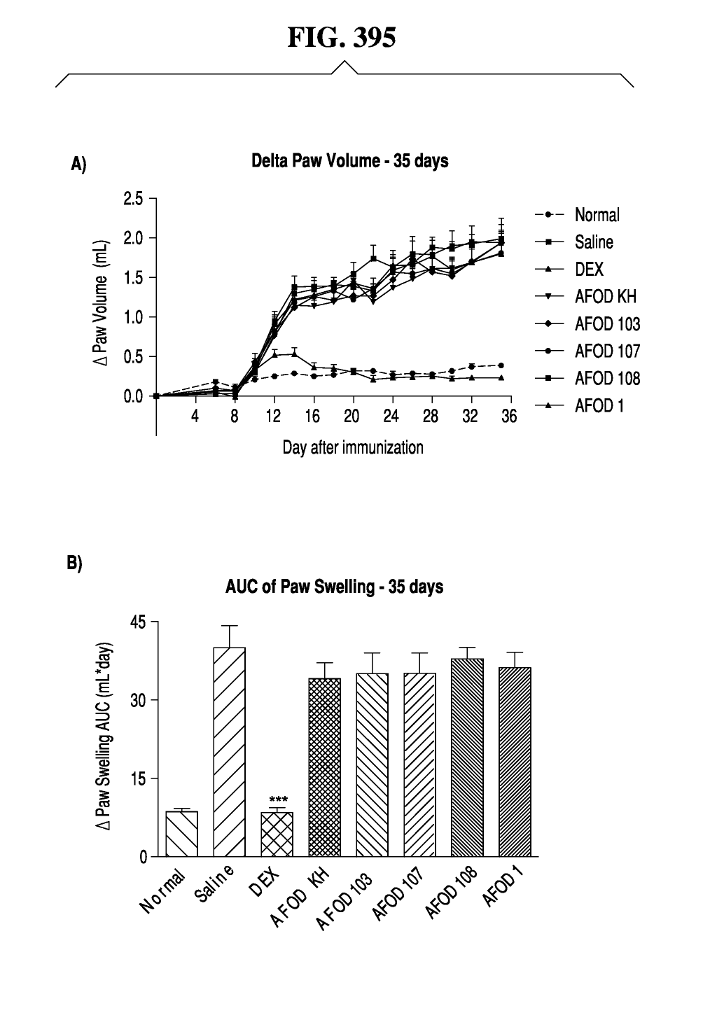

FIG. 395—Effects of AFOD KH, AFOD 103, AFOD 107, AFOD 108 and AFOD 1 on delta paw (right hind paw) volume (A) in AIA model till Day 35. AUC of delta paw volume curves were also presented (B). The delta paw volume of Dex group was significantly lower than saline group, from day 14 (***p<0.001, v.s. saline group, two-way repeated or one-way ANOVA).

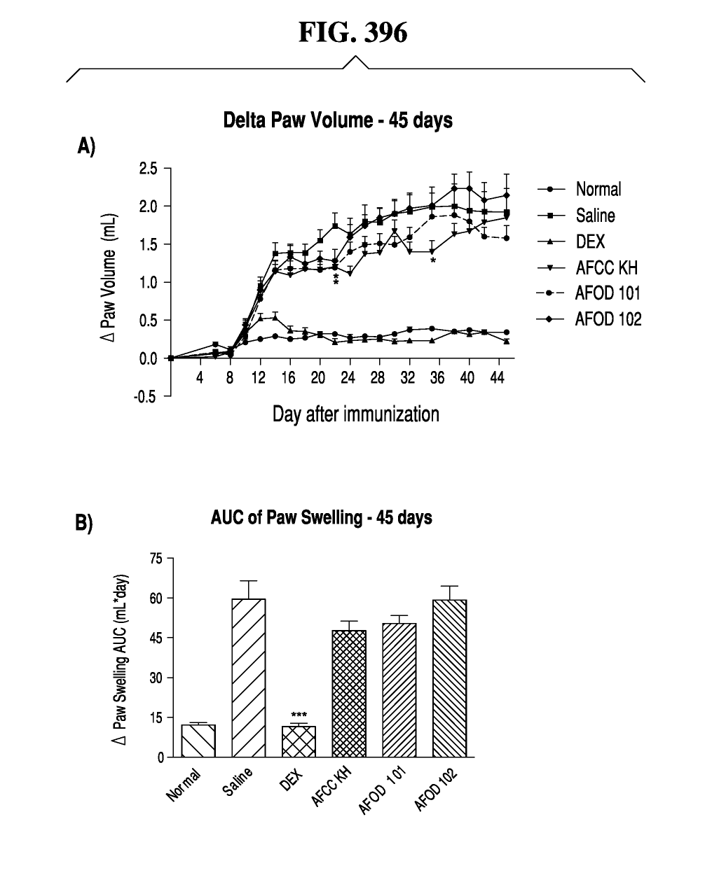

FIG. 396—Effects of AFCC KH, AFOD 101 and AFOD 102 on delta paw (right hind paw) volume (A) in AIA model till Day 45. AUC of delta paw volume curves were also presented (B). The delta paw volume of Dex group was significantly lower than saline group, from day 14 (***p<0.001, v.s. saline group, two-way repeated or one-way ANOVA).

### Arthritic Score

The arthritic scores in all the groups treated with the eight test articles were similar to that of vehicle group (FIGS. 396 & 397). Dex treatment significantly inhibited the disease development (FIGS. 396 & 397).

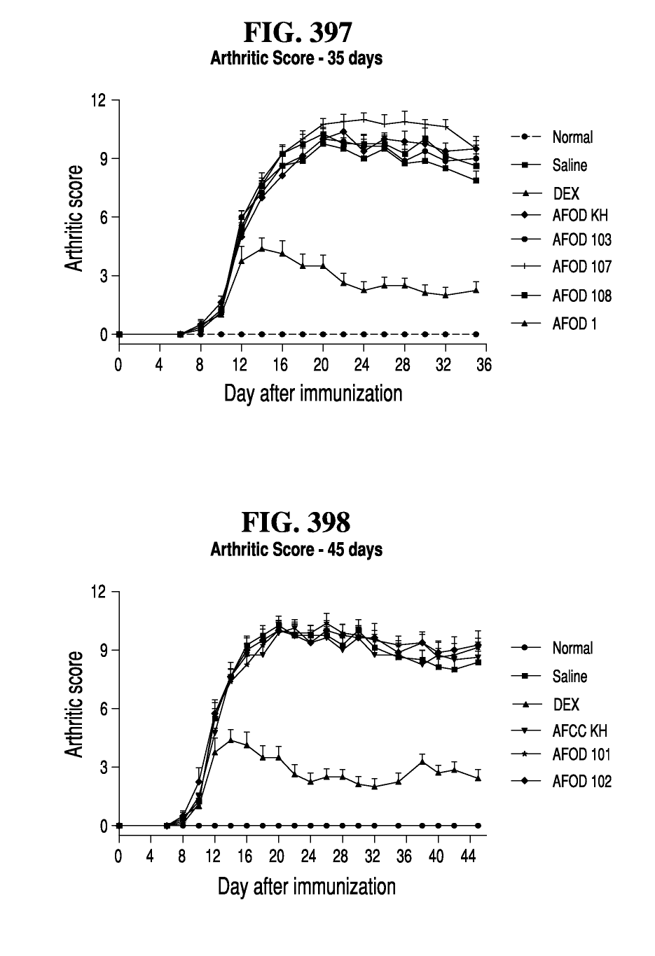

FIG. 397—Effects of AFOD KH, AFOD 103, AFOD 107, AFOD 108 and AFOD 1 on arthritic score in AIA model till day 35. The arthritic score of Dex group was significantly lower than saline group, from day 14 (p<0.01 for day 14, p<0.001 for day 16 to 35, Kruskal-Wallis test).

FIG. 398—Effects of AFCC KH, AFOD 101 and AFOD 102 on arthritic score in AIA model till Day 45. The arthritic score of Dex group was significantly lower than saline group, from day 14 (p<0.01 for day 14, p<0.001 for day 16 to 45, Kruskal-Wallis test).

### Incidence Rate

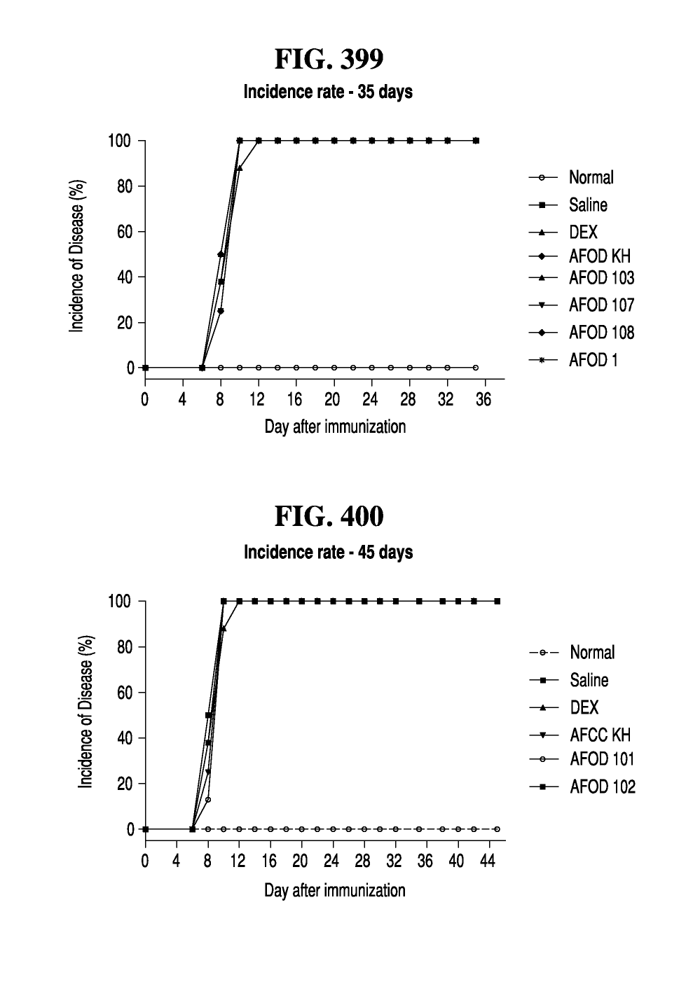

All the animals immunized with adjuvant developed arthritis at day 11 after immunization, when the treatment started, per sponsor's request. The incidence rates of all the groups remained 100% throughout the study period (FIGS. 399 & 400).

FIG. 399—Effects of AFOD KH, AFOD 103, AFOD 107, AFOD 108 and AFOD 1 on incidence rate in AIA model till day 35. The incidence rate reached 100%, 11 days after immunization. There was no change of incidence rate afterward, for all the treatment groups.

### 7.0 Conclusion

The treatment of eight test articles did not significantly affect the body weight changes compared to the saline group. The body weight of Dex group was lower than the other groups after treatment from Day 11. Overall, the treatment of eight test articles did not inhibit paw swelling significantly after 25-day or 32-day treatments. However, the group of animals treated with AFCC KH and AFOD 101 showed reduced paw swelling. Statistical analysis showed significant difference for AFCC KH and AFOD 101, but only on Day 22, 35 and Day 22 respectively, by comparing to vehicle group.
Based on the arthritic scores, all the treatments did not show significant impacts on the disease progression. Dex treatment significantly inhibited the disease development.
The incidence rate reached 100% after day 11, before the treatment started, demonstrating successful setup of the model. During the treatment from day 11 to day 45, the incidence rates in all the groups remained 100%.
Overall, the treatment of eight test articles did not inhibit paw swelling significantly after 25-day or 32-day treatments. However, the group of animals treated with AFCC KH and AFOD 101 showed reduced paw swelling. Statistical analysis showed significant difference for AFCC KH and AFOD 101, but only on Day 22, 35 and Day 22 respectively, by comparing to vehicle group.
Based on the arthritic scores, all the treatments did not show significant impacts on the disease progression. Dex treatment significantly inhibited the disease development.
The incidence rate reached 100% after day 11, before the treatment started, demonstrating successful setup of the model. During the treatment from day 11 to day 45, the incidence rates in all the groups remained 100%.

### 8.0 Reference

9.0 Debra M Meyer, Michael I Jesson, Xiong Li. Anti-inflammatory activity and neutrophil reductions mediated by the JAK1/JAK3 inhibitor CP-690,550, in rat adjuvant—induced arthritis 2010.7.1

### Study Report

### Efficacy of RAAS-8 in the HBV Mouse Hydrodynamic Injection Model

### Project Code: RASS HBV-06012012

### Study Period: Jun. 19, 2012 to Jul. 3, 2012

### 1 Introduction

Hydrodynamic injection (HDI) is an in vivo gene delivery technology. It refers to transiently transfect the mouse liver cells with a foreign gene via tail vein injection of a large volume saline containing plasmid within a few seconds. Taking the advantage of the liver-targeting manner of hydrodynamic injection, a single hydrodynamic injection of a replication-competent HBV DNA, could result in HBV replication in mouse liver shortly. This HBV hydrodynamic injection model on immunocompetent mice is a convenient and reproducible animal model for anti-HBV compound screening in vivo, which has been successfully established in WuXi ID department.

The purpose of this study is to evaluate in vivo anti-HBV efficacy of RASS 8 using the mouse hydrodynamic injection model.

### 2 Materials and Reagents

### 2.1. Animal:

Female BALB/c mice, age 6-8 weeks, between 18˜22 g.

### 2.2. Test Article:

Vehicle: normal saline.

Entecavir (ETV): supplied as powder, dissolved in normal saline prior to dosing.

AFOD-RAAS 8 (RAAS 8): provided by RAAS, 25% (blood-derived proteins) solution.

### 2.3. Reagent:

### HBV Plasmid DNA:

pcDNA3.1/HBV, prepared with Qiagen EndoFree Plasmid Giga Kit;

QIAamp 96 DNA Kit, Qiagen 51162; Universal PCR Master Mix, ABI 4324020; HBV DIG DNA probe, prepared by PCR DIG Probe Synthesis Kit, Roche 11636090910; DIG Wash and Block Buffer Set, Roche 11585762001; HBsAg ELISA kit, Kehua.

### 3 Experimental Procedure

3.1 Hydrodynamic injection and compound administration
3.1.1. From day −7 to day 0, all 5 mice in group 4 were administrated i.p./i.v. with test article daily for 8 days according to Table 14.2.
3.1.2. On day 0, all groups of mice were hydrodynamicly injected via tail vein with pcDNA3.1/HBV plasmid DNA in a volume of normal saline equal to 8% of a mouse body weight. The plasmid DNA solution for injections was prepared one day before injection and then stored in 4° C. until injection.
3.1.3. From day 0 to day 5, mice in groups 1-3 were weighed and treated with compounds or vehicle according to the regimen in Table 14.2. For groups 1 and 3, the first dosing was executed 4 hours pre HDI. For groups 2, the first dosing was executed 4 hours post HDI. For group 4, the last dosing was carried out 4 hours post HDI.
3.1.4. All mice were submandibularly bled for plasma preparation according to the design in Table 14.1.
3.1.5. All mice were sacrificed and dissected to obtain livers (two pieces of left lobe, one piece of middle lobe and one piece of right lobe) according to the regimen in table 14.1. Isolated livers were snap frozen in liquid nitrogen immediately upon collected.

TABLE 14.2
Schedule for Compound administration

| | | Day | | | | | | | | | | | | | | |
|---|---|---|---|---|---|---|---|---|---|---|---|---|---|---|---|---|
| group | | −7 | −6 | −5 | −4 | −3 | −2 | −1 | 0 | 1 | 2 | 3 | 4 | 5 | 6 | 7 |
| 4 | am | 0.2 ml, | 0.4 ml | 0.2 ml, | 0.4 ml | 0.2 ml | 0.4 ml | 0.4 ml | HDI*, | No | No | No | No | No | No | No |
| | | IV | IP | IV | IP | IV | IP | IP | IV | | | | | | | |
| | pm | No | No | No | No | No | No | No | 0.5 ml | No | No | No | No | No | No | No |
| | | | | | | | | | IP | | | | | | | |
| 2 | am | No | No | No | No | No | No | No | HDI, | 0.2 ml | 0.5 ml | 0.2 ml | 0.5 ml | No | No | No |
| | | | | | | | | | IV | IV | IP | IV | IP | | | |
| | pm | No | No | No | No | No | No | No | 0.5 ml | 0.3 ml | No | 0.3 ml | No | No | No | No |
| | | | | | | | | | IP | IP | | IP | | | | |
| 1 | am | No | No | No | No | No | No | No | 0.5 ml | 0.2 ml | 0.5 ml | 0.2 ml | 0.5 ml | No | No | No |
| | | | | | | | | | IP | IV | IP | IV | IP | | | |
| | pm | No | No | No | No | No | No | No | HDI, | 0.3 ml | No | 0.3 ml | No | No | No | No |
| | | | | | | | | | IV | IP | | IP | | | | |

HDI*: hydrodynamic injection

### 3.2 Sample Analysis

### 3.2.1 Detect HBV DNA Replication Level in Plasma

3.2.1.1 Isolate DNA from 50 μl plasma using QIAamp 96 DNA Blood Kit. DNA was eluted with 120 μl ddH₂O.
3.2.1.2. Run qPCR for HBV DNA quantification.
   a) Dilute HBV plasmid standard by 10-fold from 10⁷ copies/μl to 10 copies/μl.
   b) Prepare qPCR mix as shown below.

| | | | Volume for | |
|---|---|---|---|---|
| PCR reagents | Volume | | 100 Reactions | |
| DEPC Water | 1.1 | μl | 110 | μl |
| Taqman Universal Master Mix (2X) | 12.5 | μl | 1250 | μl |
| HBV Primer Forward (50 μM) | 0.2 | μl | 20 | μl |
| HBV Primer Reverse (50 μM) | 0.2 | μl | 20 | μl |
| HBV Probe (5 μM) | 1 | μl | 100 | μl |
| Total | 15 | μl | 1500 | μl |

c) Add 15 μl/well PCR mix to 96-well optical reaction plates.
d) Add 10 μl of the diluted plasmid standard.
e) Transfer 10 μl of the extracted DNA to the other wells. Seal the plates with optical adhesive film. Mix and centrifuge.
f) Place the plates into qPCR machine and run the program according to the table blow.

| | | |
|---|---|---|
| 50° C. | 2 min | 1 cycle |
| 95° C. | 10 min | 1 cycle |
| 95° C. | 15 s | 40 cycle |
| 60° C. | 60 s | |

g)

To eliminate the influence of input HBV plasmid, primers and probe targeting HBV sequence which detect newly replicated HBV DNA and input HBV plasmid DNA and targeting pcDNA3.1 plasmid backbone sequence which only detect the input plasmid DNA were used to do real-time PCR, respectively.

HBV DNA quantity=DNA determined by HBV primer-DNA determined by plasmid primer.

### 3.2.2 Detect HBsAg Level in Plasma

Dilute the plasma 500 fold;

Detect HBsAg level in 50 μl diluted plasma by using HBsAg ELISA kit.

### 3.2.3 Detect HBV Intermediate DNA Level in Livers

### 3.2.3.1 Liver DNA Isolation

a) Homogenize the liver tissue with Qiagen Tissue Lyser in 10 mM Tris.HCl, 10 mM EDTA, pH7.5.
b) Spin samples. Transfer the supernatant to a new tube containing equal volume of 2× proteinase K digestion buffer. Incubate at 50° C. for 3 hours.
c) Extract with phenol: choroform: Isoamyl alcohol.
d) Transfer the upper phase to new tubes, add RNase A and incubate at 37° C. for 30 min.
e) Extract with phenol: choroform: Isoamyl alcohol.
f) Transfer the upper phase to new microfuge tubes, add 0.7-1 volume of isopropanol, add GlycoBlue Coprecipitant to 50 μg/mL, incubate at −20° C. for 30 min.
g) Centrifuge (12000 g, 10 min) to precipitate DNA.
h) Wash the precipitate with 70% ethanol. Dissolve it in 25 μl ddH₂O. Store DNA at −20° C. until use.

3.2.3.2 qPCR for HBV DNA quantification with total liver DNA.

The total liver DNA was diluted to 10 ng/μl. Use 10 μl diluted sample to run real-time PCR.

HBV DNA quantity=DNA determined by HBV primer-DNA determined by plasmid primer.

3.2.3.3 Southern blot to detect HBV intermediate DNA level in livers.

a) Load 50 μg DNA for each sample. Run 1.2% agarose gel in 1×TAE.
b) After denaturing the gel with 0.25 M HCl at RT, neutralize the gel with neutralizing buffer.
c) Transfer the DNA form the gel to a pre-wet positively charged nylon membrane by upward capillary transfer overnight.
d) Remove the nylon membrane from the gel transfer assembly, UV cross-link the membrane (700 Microjoules/cm²), then wash it in 2×SSC for 5 min. Place the membrane at RT until dry.
e) Prehybridize membrane for 1 hour with hybridization buffer.
f) Pour off hybridization solution, and add the hybridization/pre-heated probe mixture, overnight
g) After hybridization and stringency washes, rinse membrane briefly in washing buffer.
h) Incubate the membrane in blocking solution, then in Antibody solution.
i) After wash in washing buffer, equilibrate in Detection buffer.
j) Place membrane with DNA side facing up on a development folder (or hybridization bag) and apply CDP-Star, until the membrane is evenly soaked. Immediately cover the membrane with the second sheet of the folder to spread the substrate evenly and without air bubbles over the membrane.
k) Squeeze out excess liquid and seal the edges of the development folder. Expose to X-ray film.
l) Expose to X-ray film at 15-25° C.

### 4 Results and Discussion

To investigate the effect of tested compounds on HBV replication in hydrodynamic model, the level of HBV DNA in plasma was analyzed by real-time PCR method (FIG. 1). Because the injected HBV plasmid DNA can also be detected by the primers targeting to HBV sequence, the primers and probe targeting the backbone sequence of pcDNA3.1 vector were designed and used for real-time PCR to eliminate the influence of residual plasmid in blood. The HBV quantity was calculated by the quantity determined by primers targeting HBV sequence subtracted by quantity determined by primers targeting the plasmid backbone sequence.

The results indicated that RASS 8 significantly inhibited the HBV replication by therapeutic or prophylactic treatment in a time-dependent manner post HDI. On day 1, RASS 8 therapeutic treatment showed ˜23% inhibition and RASS 8 prophylactic treatment showed ˜37% inhibition to HBV replication. On day 3 and day 4, the inhibition percentage to HBV replication by RASS 8 therapeutic, or prophylactic treatment was >99%, which is statistically significant. On day 5, RASS 8 therapeutic treatment caused ˜93% inhibition while its prophylactic treatment made almost 100% inhibition. The HBV level in both RAAS 8 prophylactic and therapeutic groups recovered a little on day 7 compared to the data on day 5. As a reference compound for the HBV HDI model, entecavir had significant inhibition to the HBV replication in the therapeutically-treated mice from day 3 post HDI to the end of experiment.

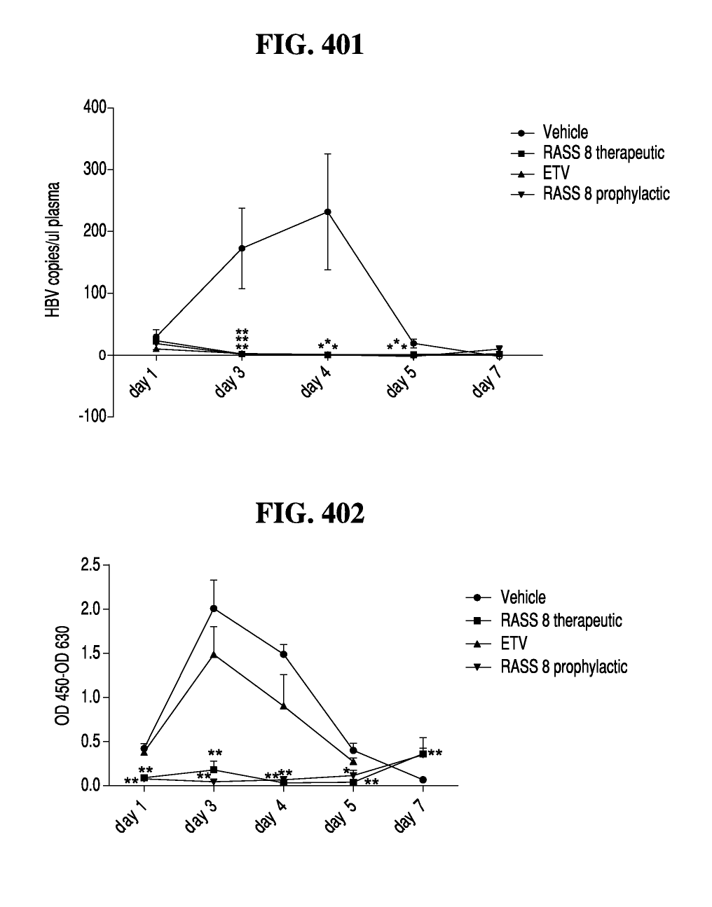

FIG. 401. Efficacy of therapeutic treatment or prophylactic treatment of RAAS 8 or ETV on in vivo HBV replication in HBV mouse HDI model. The total DNA was isolated from plasma by QIAamp 96 DNA Blood Kit. The HBV viral load in plasma during the course of the experiment was quantified by real-time PCR. Data is expressed as mean±SE. *P<0.05, **P<0.01 by Student's t-test.

Secreted HBV surface proteins are also important index for HBV replication. HBsAg level in plasma was detected by ELISA method (FIG. 2). Both RASS 8 therapeutic and prophylactic treatment had a significant inhibitory effect on HBsAg level in plasma within 5 days post HBV HDI while ETV didn't have significant inhibition to the HBsAg generation, suggesting that the in vivo effect of RAAS 8 on the in vivo HBV replication may be through a different mechanism from the entecavir.

FIG. 402. Effect of prophylactic treatment or therapeutic treatment of RAAS 8 or ETV on the HBsAg in mouse blood. The HBsAg level in plasma during the course of the experiment was determined by HBsAg ELISA kit. Data is expressed as mean±SE. *P<0.05, **P<0.01 by Student's t-test.

Hepatitis B virus is a member of the hepadnavirus family, which replicates in livers and depends on liver specific factors. Thus, the existence of intermediate DNA in livers is a direct evidence for HBV replication in livers. To quantify the intermediate HBV DNA in livers, the total DNA was isolated from liver and HBV DNA level was determined by real-time PCR (FIG. 3). ETV, as a positive control, significantly decreased the HBV intermediate DNA in liver on day 5. Similar to ETV, RASS 8 prophylactic treatment had a significant inhibition on the replication of HBV intermediate DNA in livers on day 7. In comparison to the prophylactic treatment of RAAS 8, its therapeutic treatment caused significant but to less extent inhibition to the liver HBV replication by real time PCR (FIG. 3).

The HBV quantity determined by real-time PCR is total copy number of rcDNA, dsDNA and ssDNA. To separate and visualize rcDNA, dsDNA and ssDNA, southern blot was performed (FIG. 4). The major form of HBV replication intermediate DNA was ssDNA, which was consistent with report in literatures. Due to the limitation of DIG DNA probe sensitivity, we were not able to detect rcDNA or dsDNA. ssDNA decreased dramatically after RASS 8 prophylactic treatment or ETV treatment (FIG. 4), which confirms the result by real-time PCR (FIG. 3).

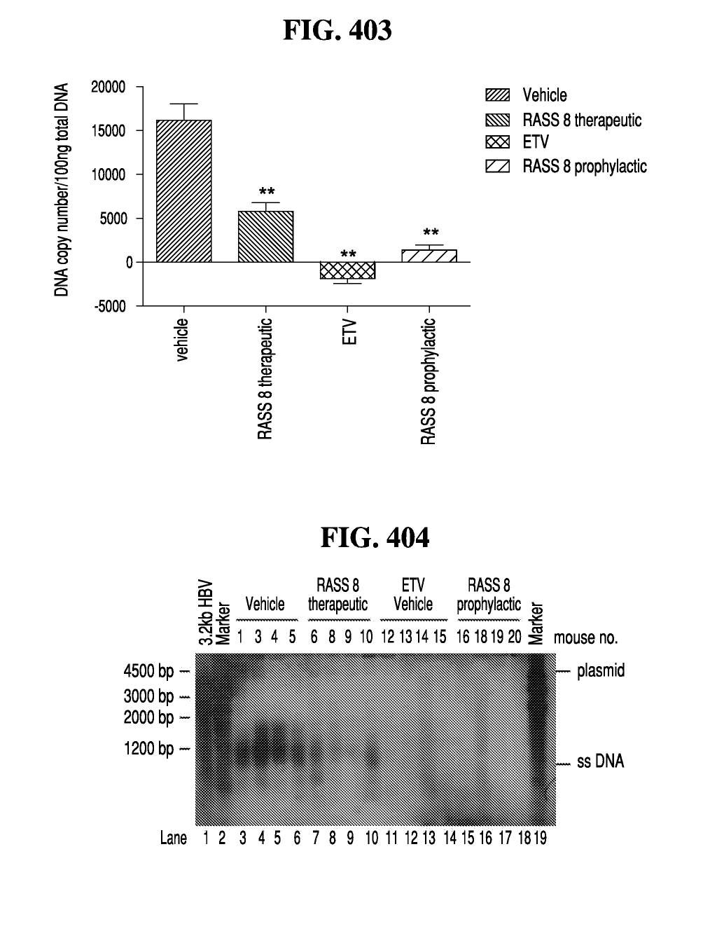

FIG. 403. Effect of prophylactic treatment or therapeutic treatment of RAAS 8 or ETV on the intermediate HBV replication in the mouse livers by qPCR. Mice in ETV group were sacrificed on day 5 and mice in the other three groups were sacrificed on day 7 post HDI. Liver DNA was isolated and subjected to real-time PCR to quantify the level of HBV replication intermediate DNA. Data is expressed as mean±SE. **P<0.01 by Student's t-test.

FIG. 404. Southern blot determination of intermediate HBV DNA in mouse livers. 50 μg total DNA each was subjected to southern blot. Lane 1 is 3.2 kb fragment of HBV plasmid (100 pg). Lane 2 and lane 19 are DNA makers. Lanes 3 to 18 are samples.

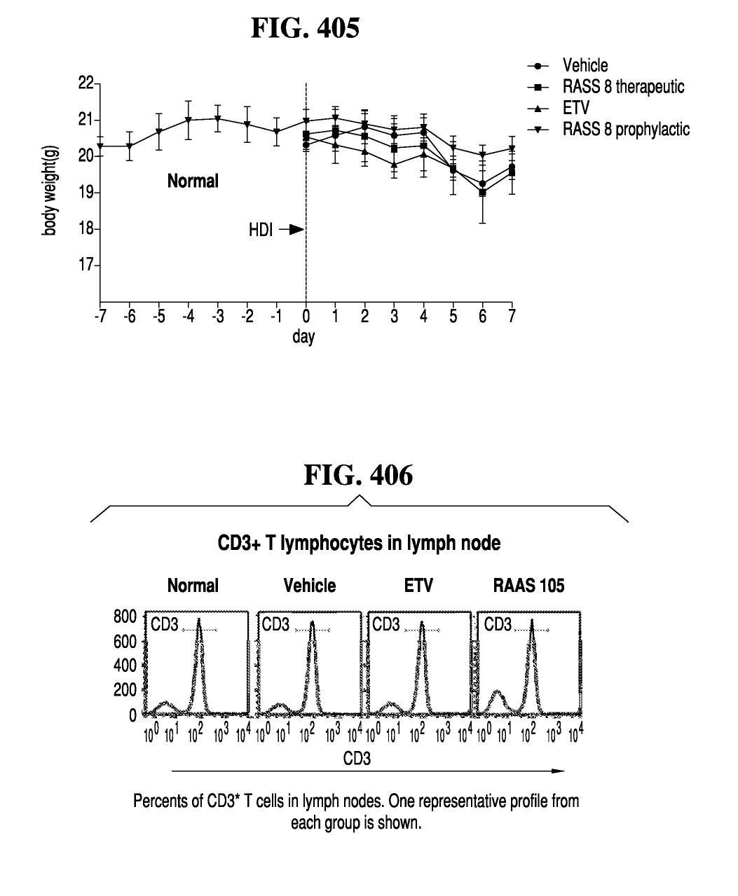

FIG. 405. The body weights of mice treated with vehicle or indicated compounds during the course of experiment

In summary, the RAAS 8 significantly inhibited HBV DNA replication by prophylactic or therapeutic treatment in the current study with the mouse HDI model. Impressively the prophylactic treatment with RAAS 8 displayed stronger inhibition to the HBV replication than its therapeutic treatment although we need more experiment to understand this phenomenon. In this study only 5 mice were used in each group. Thus the result may need to be confirmed by using more animals. In addition a well-designed mechanism study may be required to clarify how the RAAS 8 protein functions against HBV infection.

### FACS Results RAAS HBV Model Study in Mice

### Update 2, Prophylactic 105

The In-Vitro study of the mice to prove the efficacy of AFOD RAAS 105® in stopping the replication of the HBV virus on day 5 and completely eliminate all the Hepatitis B surface antigen also on day 5, then four mice from each group of the prophylaxis and the therapeutic treatment have been analyzed to find out the mechanism and the cell population in the mice of each group. Vehicle control, Positive control, Negative control, Prophylactic treated group and Therapeutically treated group.

We found that the change of the immune cell population in lymph node, spleen and the peripheral blood has tremendously increased of none T and none B lymphocytes, which cannot be recognized by current detection methods and the inventor concludes that these are KH new found good healthy cells, like the dragon cell in which the RNA synthesizes good proteins that: 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging, TO CURE THE HEPATITIS B VIRUS.

CD3+ T lymphocytes in lymph node of RAAS 105 reduced compared to the vehicle and positive group. In another study of the breast cancer we have found that the lymphocytes of those nude mice with cancer have increased this means that all the solid cancers or blood cancers like lymphoma and leukemia will have lymphocytes in the organs and blood. So the inventor concludes that the cancer patient already in the beginning stage have the lymphocyte cancer cells.

FIG. 406—CD3+ T lymphocytes in lymph node

T lymphocytes subsets in lymph node CD4 is lower than the vehicle and positive control and CD8 is higher.

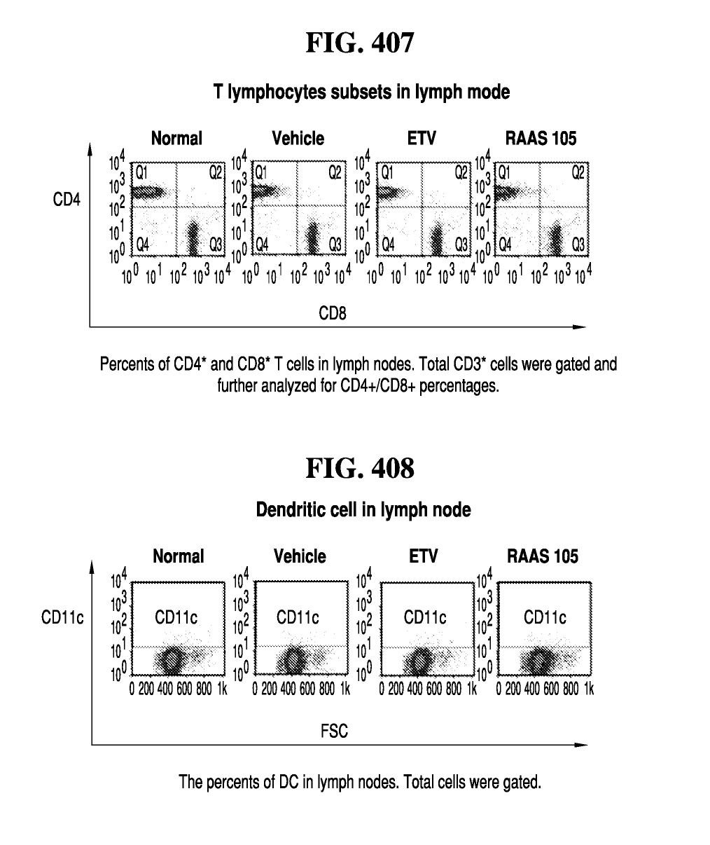

FIG. 407—T lymphocytes subsets in lymph node

Dendritic cell in lymph node is AFOD RAAS 105® is higher than the vehicle and the positive control

FIG. 408—Dendritic cell in lymph node

CD4+ T lymphocytes subsets in lymph node is lower in AFOD RAAS 105®

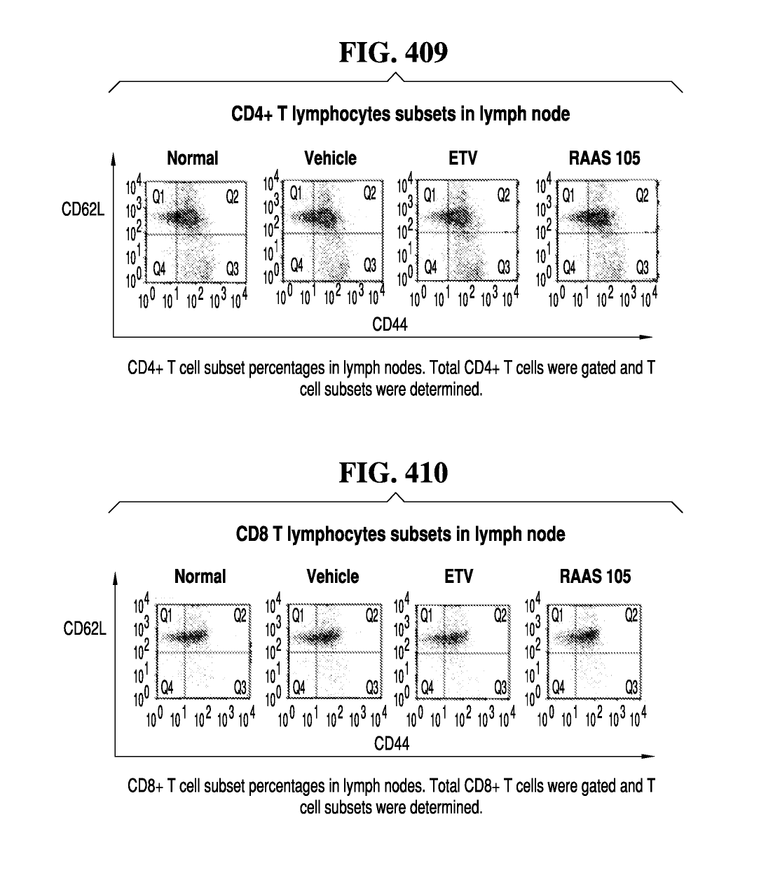

FIG. 409—CD4+ T lymphocytes subsets in lymph node

CD8 T lymphocytes subsets in lymph node is higher in AFOD RAAS 105®

FIG. 410—CD8 T lymphocytes subsets in lymph node

Macrophages/Granulocytes in lymph node is higher in granulocytes and lower in Macrophage.

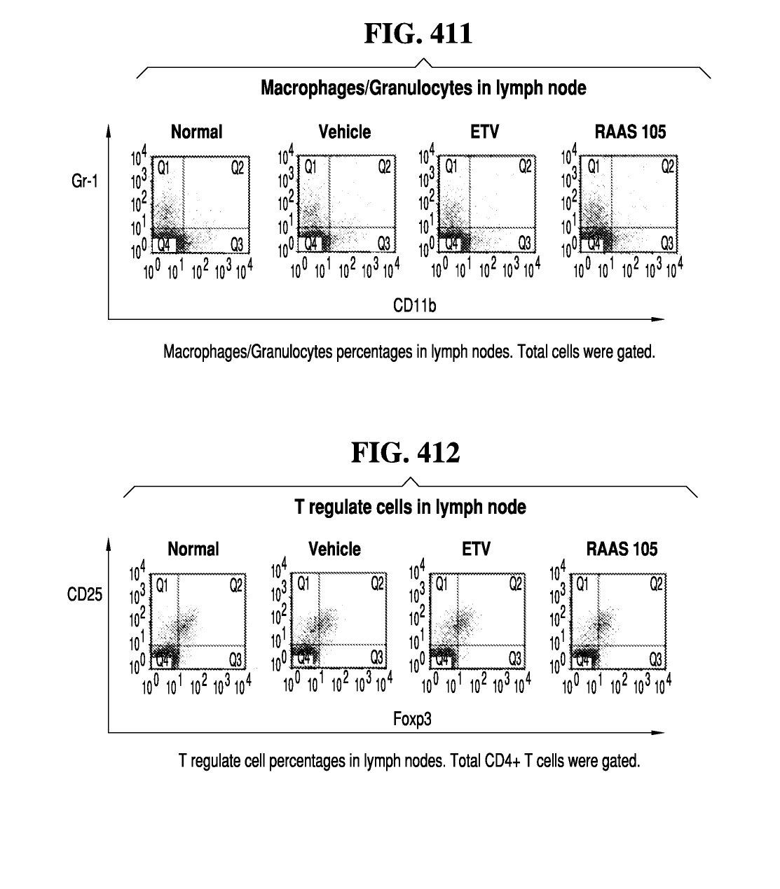

FIG. 411—Macrophage/Granulocytes in lymph node

T regulate cells in lymph node slightly increase in AFOD RAAS 105®

FIG. 412—T regulate cells in lymph node

T lymphocytes/B lymphocytes in spleen is higher than the vehicle control in AFOD RAAS 105® and much higher than in the normal group.

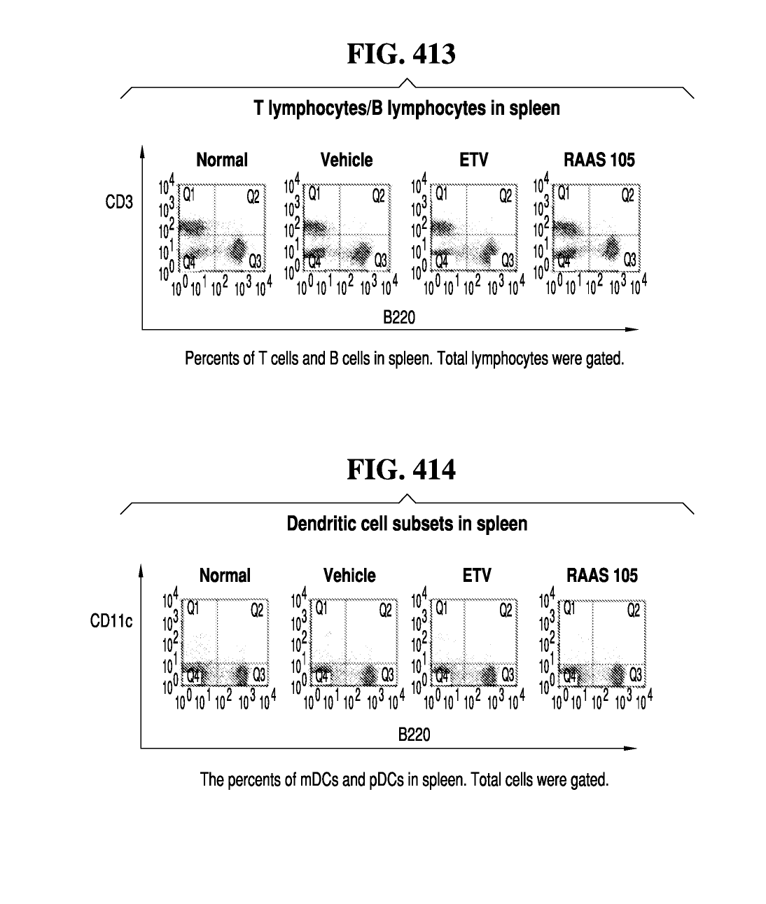

FIG. 413—T lymphocytes/B lymphocytes in spleen

T lymphocytes subsets in spleen is slightly higher in CD8 and slightly lower in CD3.

FIG. 414—Dendritic cell subsets in spleen

Dendritic cell subsets in spleen is higher for AFOD RAAS 105®

CD4+ T lymphocytes subsets is lower in AFOD RAAS 105®

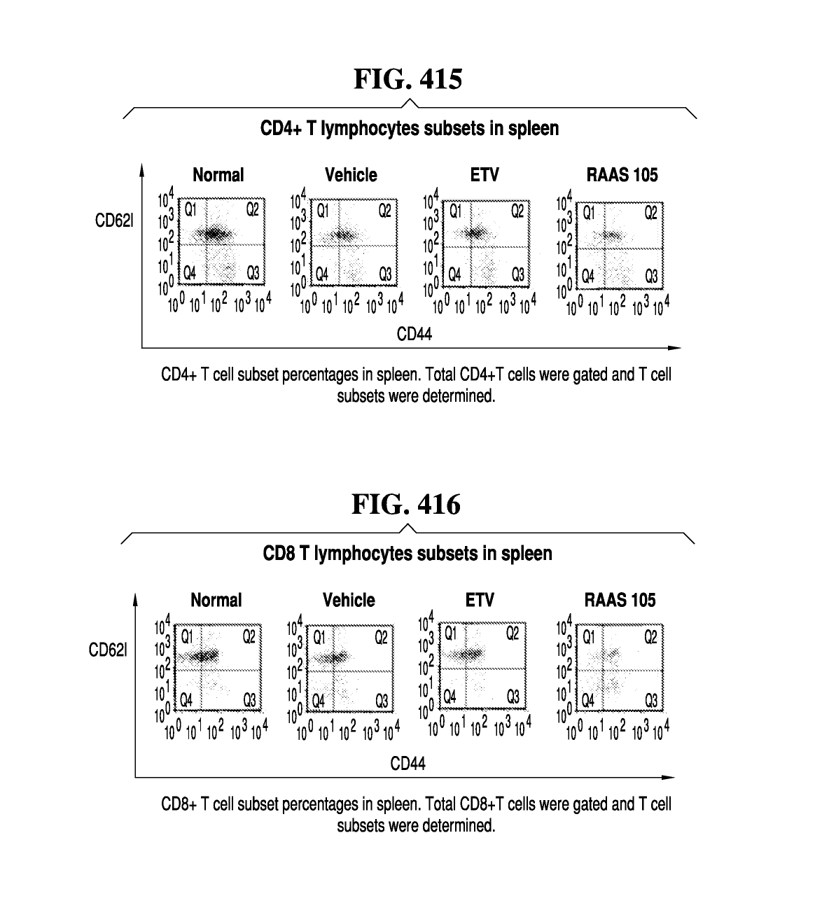

FIG. 415—CD4+ T lymphocytes subsets in spleen CD8 T lymphocytes subsets in spleen is lower in AFOD RAAS 105®

FIG. 416—CD8 T lymphocytes subsets in spleen

Macrophages subsets in spleen is the same as vehicle in AFOD RAAS 105®

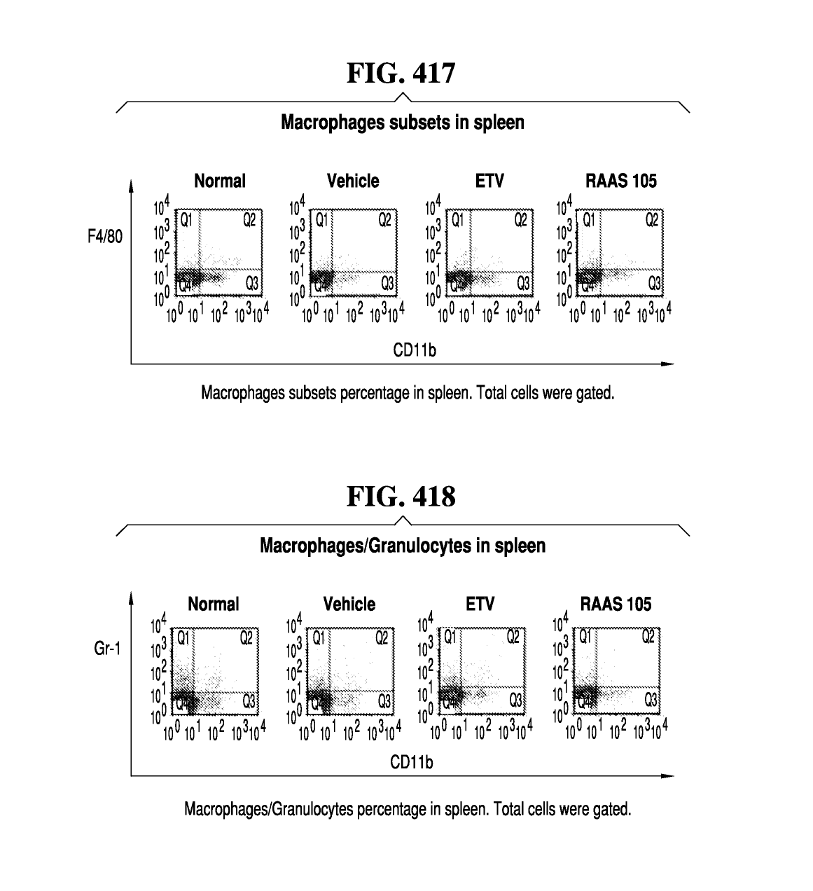

FIG. 417—Macrophages subsets in spleen

Macrophage/Granulocytes in spleen is lower in AFOD RAAS 105® to compare with vehicle. AFOD RAAS 105® does not compare with the positive control as ETV can stop the replication of the HBV virus but CANNOT eliminate the Hepatitis B surface antigen in mice. Therefore the comparison with the positive control is invalid.

FIG. 418—Macrophages/Granulocytes in spleen

T regulate cells has approximately a 40% increase in AFOD RAAS 105®

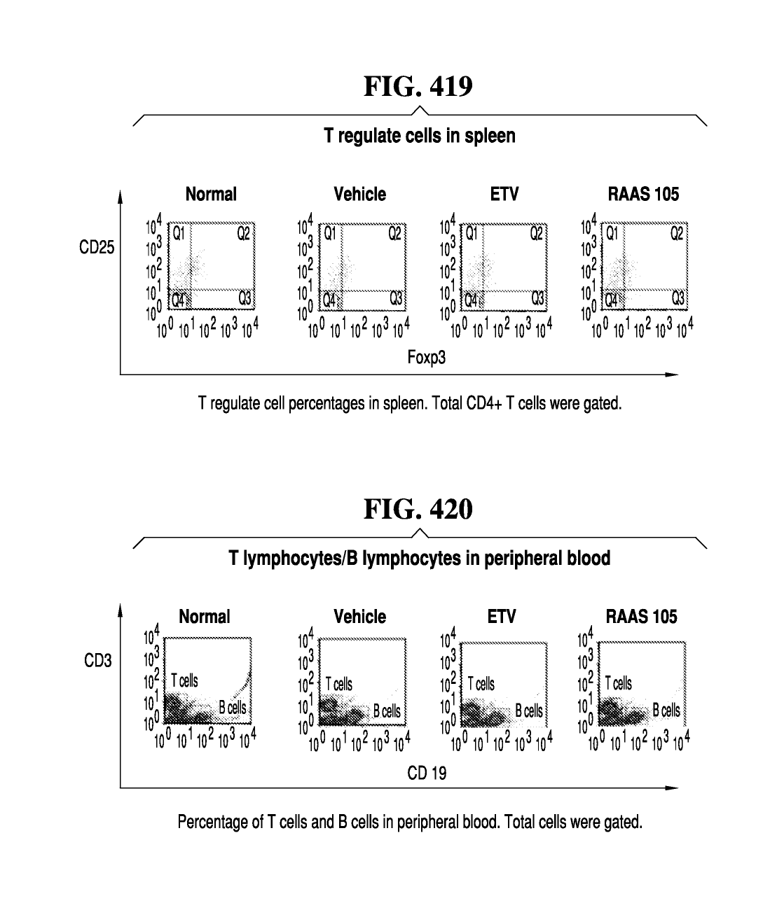

FIG. 419—T regulate cells in spleen

T lymphocytes/B lymphocytes in peripheral blood has a25% increase in T cell and 30% decrease in B cells in AFOD RAAS 105®

FIG. 420—T lymphocytes/B lymphocytes in peripheral blood

T lymphocytes subsets in peripheral blood is 15% lower in AFOD RAAS 105®

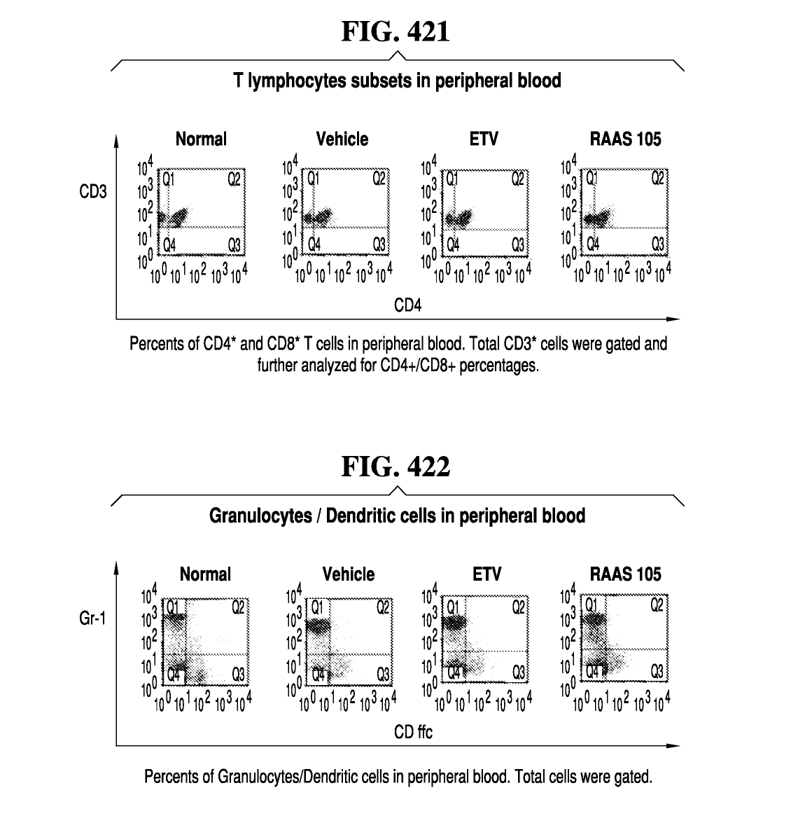

FIG. 421—T lymphocytes subsets in peripheral blood

Granulocytes/Dendritic cells in peripheral blood 55% increase in AFOD RAAS 105®

FIG. 422—Granulocytes/Dendritic cells in peripheral blood

Monocytes in peripheral blood is 33% higher in AFOD RAAS 105®

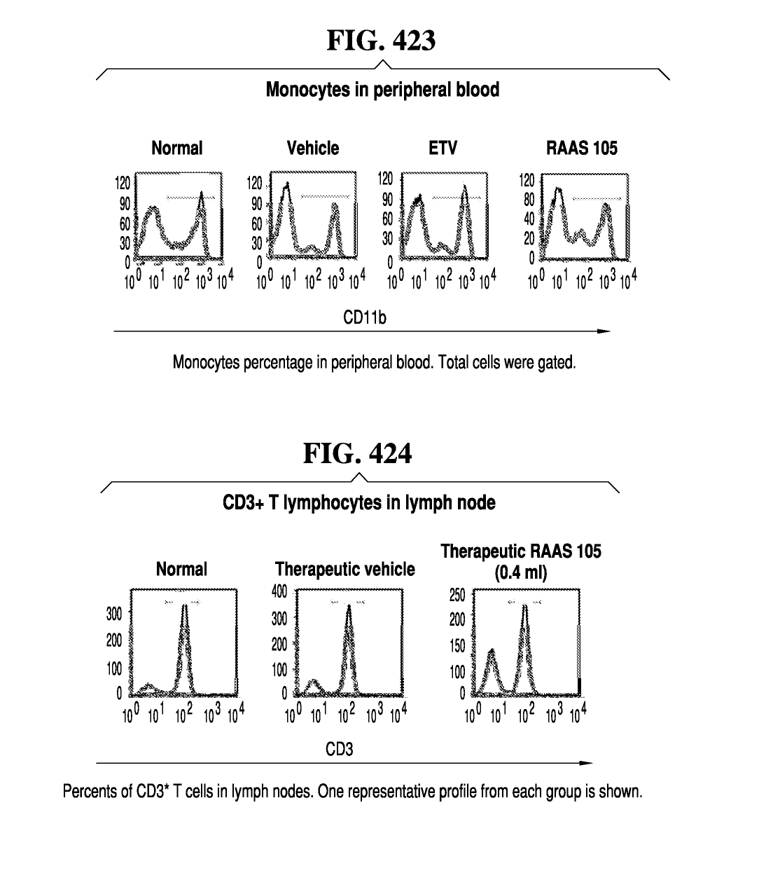

FIG. 423—Monocytes in peripheral blood

FACS results (partial) RAAS HBV model study in mice for the Therapeutic group

CD3+ T lymphocytes in lymph node is 33% lower in AFOD RAAS 105®

FIG. 424—CD3+ T lymphocytes in lymph node

T lymphocytes subsets in lymph node is 5% lower in CD4 and 18% higher CD8 in AFOD RAAS 105®

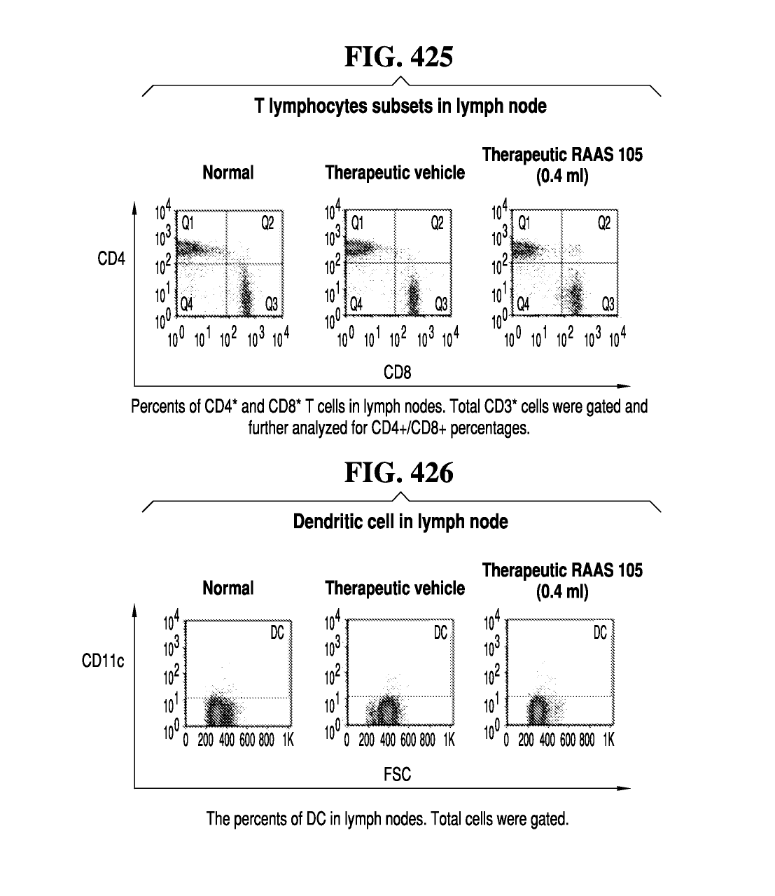

FIG. 425—T lymphocytes subsets in lymph node

Dendritic cell in lymph node is 10% higher in AFOD RAAS 105®

FIG. 426—Dendritic cell in lymph node

CD4+ T lymphocytes subsets in lymph node is 5,000% lower in AFOD RAAS 105®

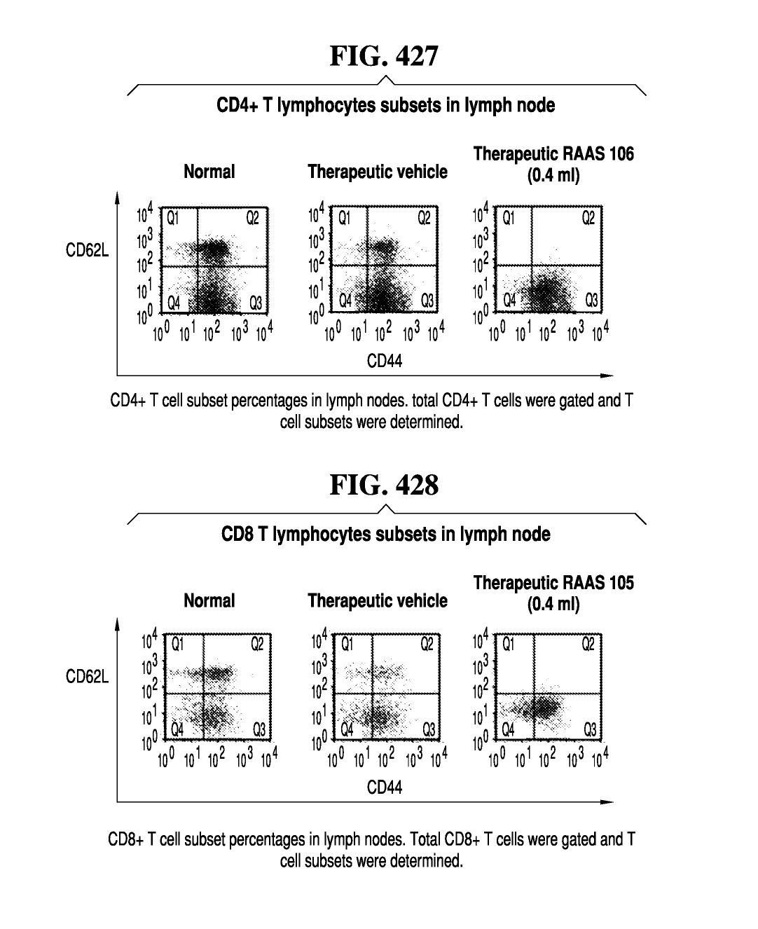

FIG. 427—CD4+ T lymphocytes subsets in lymph node

CD8 T lymphocytes subsets in lymph node is 846% lower in AFOD RAAS 105®

FIG. 428—CD8 T lymphocytes subsets in lymph node

Macrophages/Granulocytes in lymph node is 57% increase in AFOD RAAS 105®

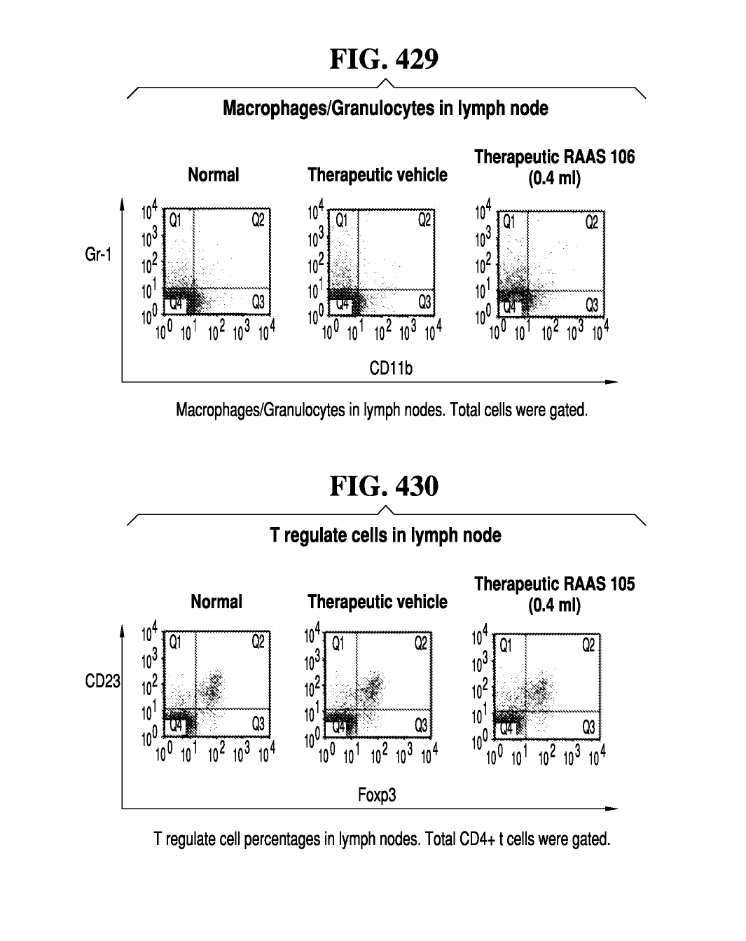

FIG. 429—Macrophages/Granulocytes in lymph node

T regulate cells in lymph node is 28% higher in AFOD RAAS 105®

FIG. 430—T regulate cells in lymph node

T lymphocytes/B lymphocytes in spleen is 67% lower T cells, 69% lower B cells and 170% increase in non T/non B cell (KH good healthy cells, like dragon cell under different patent application for new cell discovery) in AFOD RAAS 105®

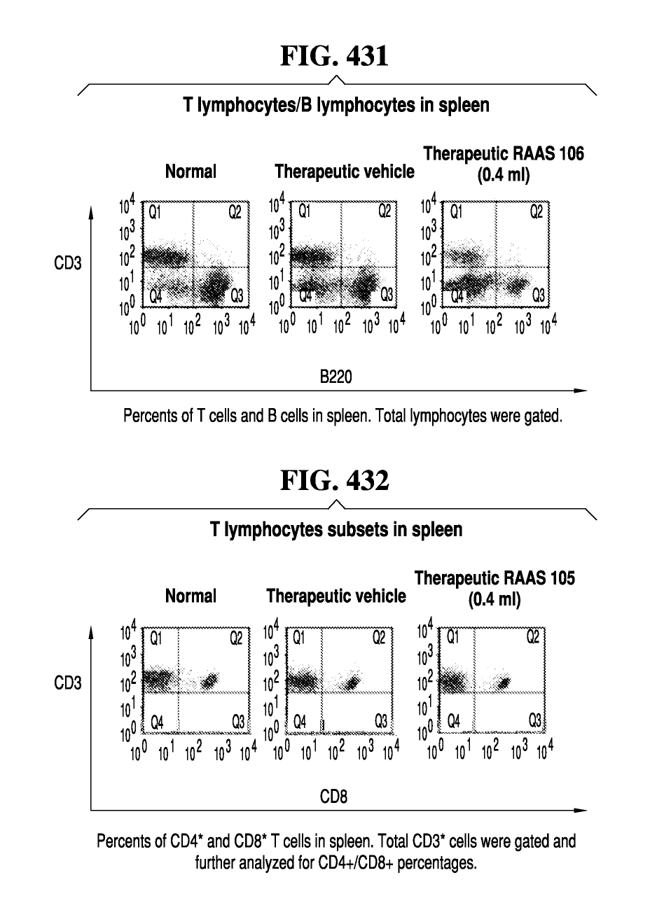

FIG. 431—T lymphocytes/B lymphocytes in spleen

T lymphocytes subsets in spleen is 13% lower in AFOD RAAS 105®

FIG. 432—T lymphocytes subsets in spleen

Dendritic cell subsets in spleen 62% lower in RAAS AFOD 105®

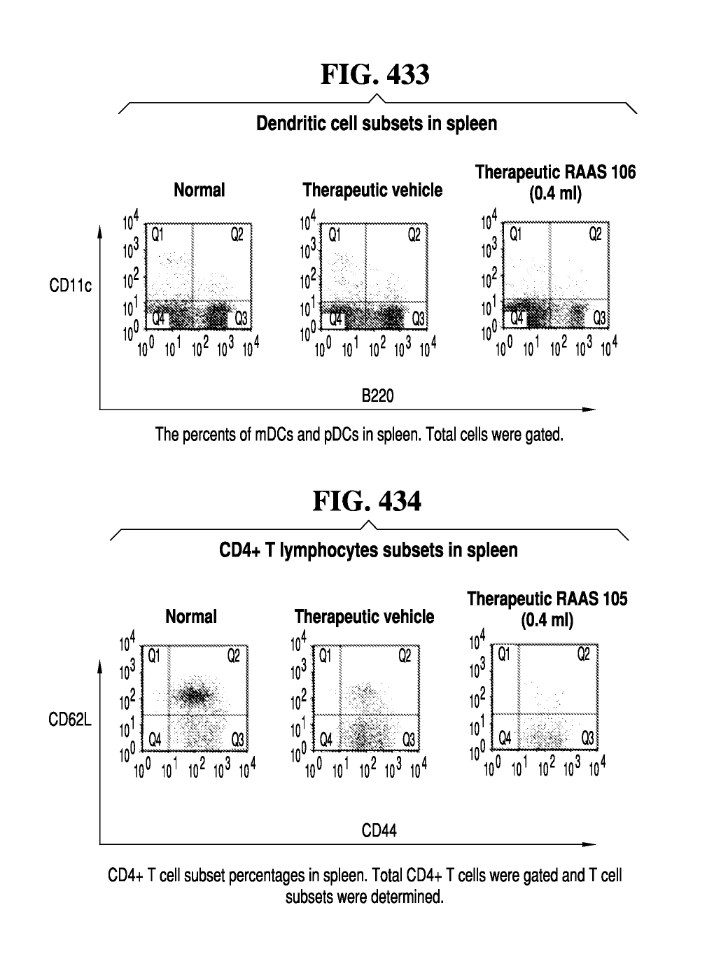

FIG. 433—Dendritic cell subsets in spleen

CD4+ T lymphocytes subsets in spleen is 80% lower in AFOD RAAS 105®

FIG. 434—CD4+ T lymphocytes subsets in spleen

CD8 T lymphocytes subsets in spleen is 85% lower in AFOD RAAS 105®

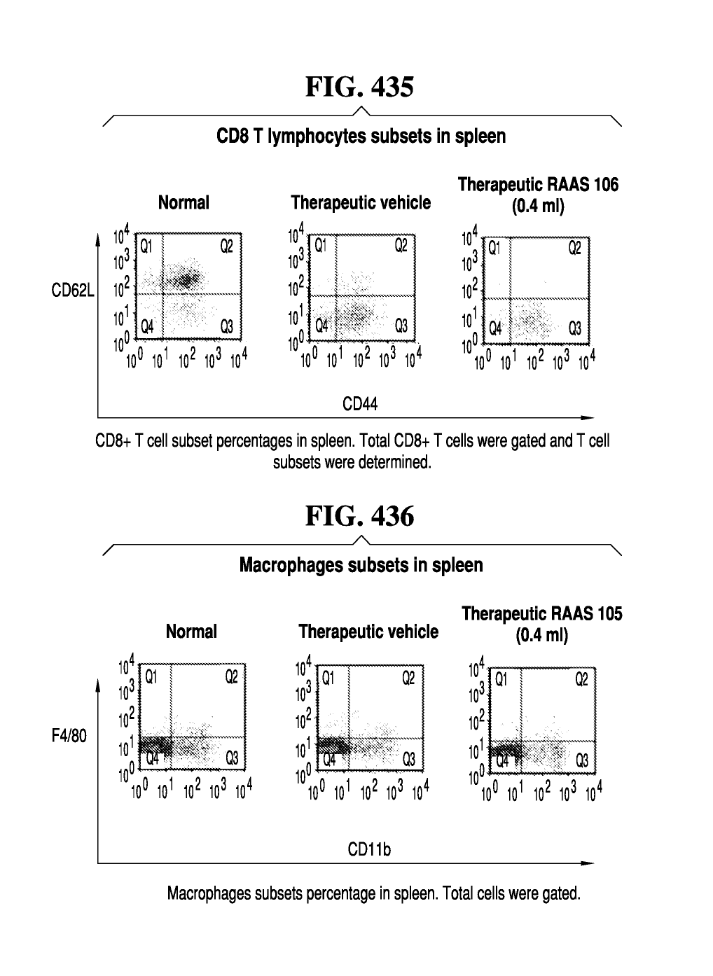

FIG. 435—CD8 T lymphocytes subsets in spleen

Macrophage subsets in spleen is 39% lower in AFOD RAAS 105®

FIG. 436—Macrophages subsets in spleen

Macrophages/Granulocytes in spleen is 18% lower in AFOD RAAS 105®

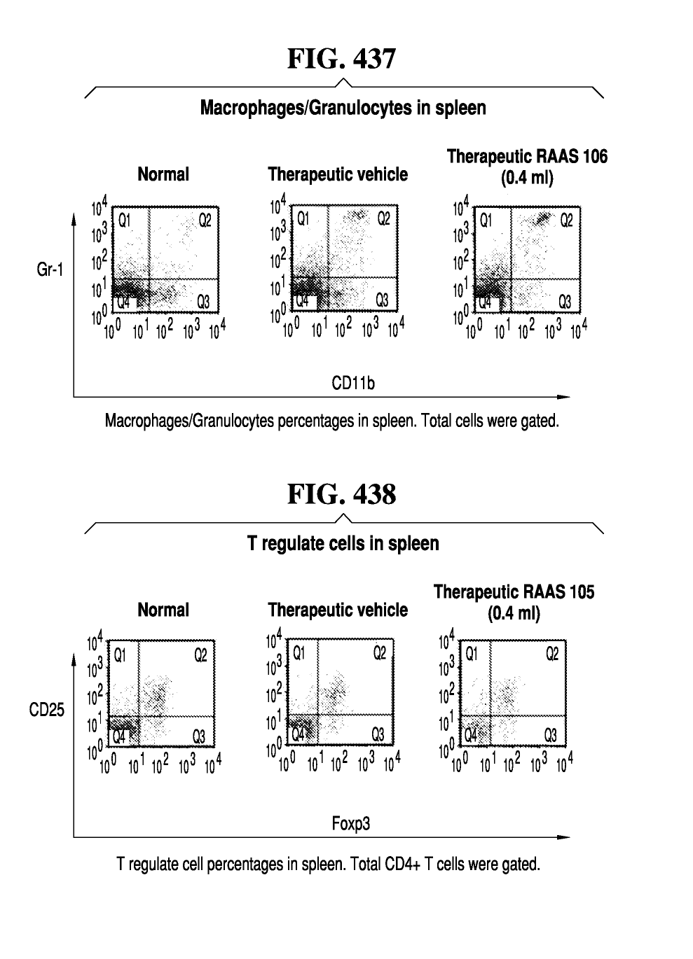

FIG. 437—Macrophages/Granulocytes in spleen

T regulate cells in spleen 100% higher in AFOD RAAS 105®

FIG. 438—T regulate cells in spleen

T lymphocytes/B lymphocytes in peripheral blood is 29% lower of T cells and 60% lower of B cells in AFOD RAAS 105®

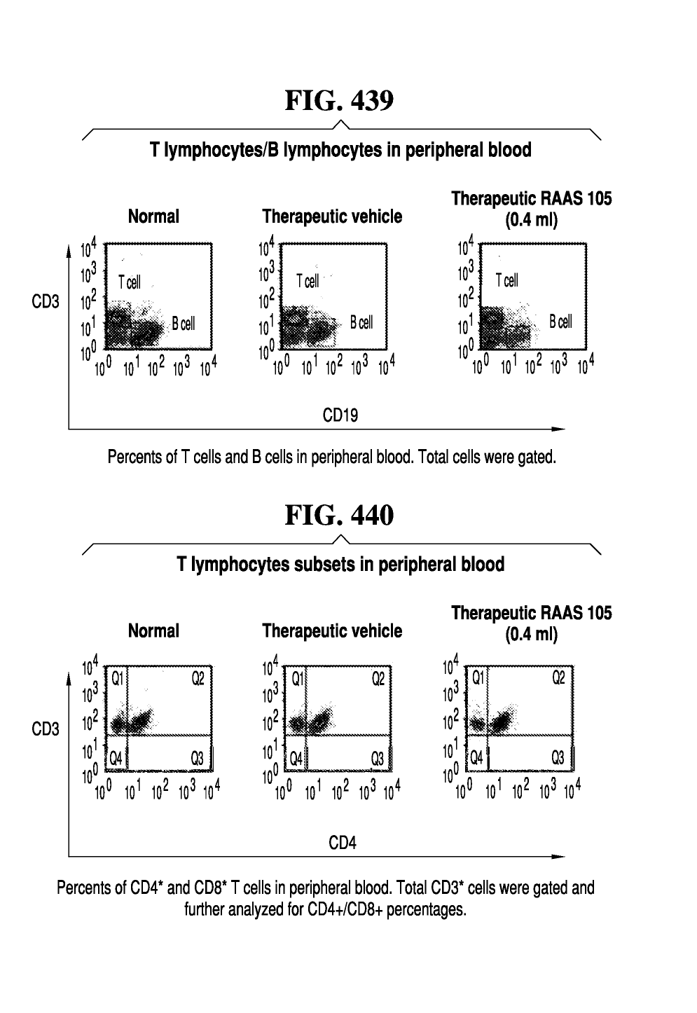

FIG. 439—T lymphocytes/B lymphocytes in peripheral blood

T lymphocytes subsets in peripheral blood is 4% higher in AFOD RAAS 105®

FIG. 440—T lymphocytes subsets in peripheral blood

Granulocytes/Dendritic cells in peripheral blood is 30% higher in AFOD RAAS 105®

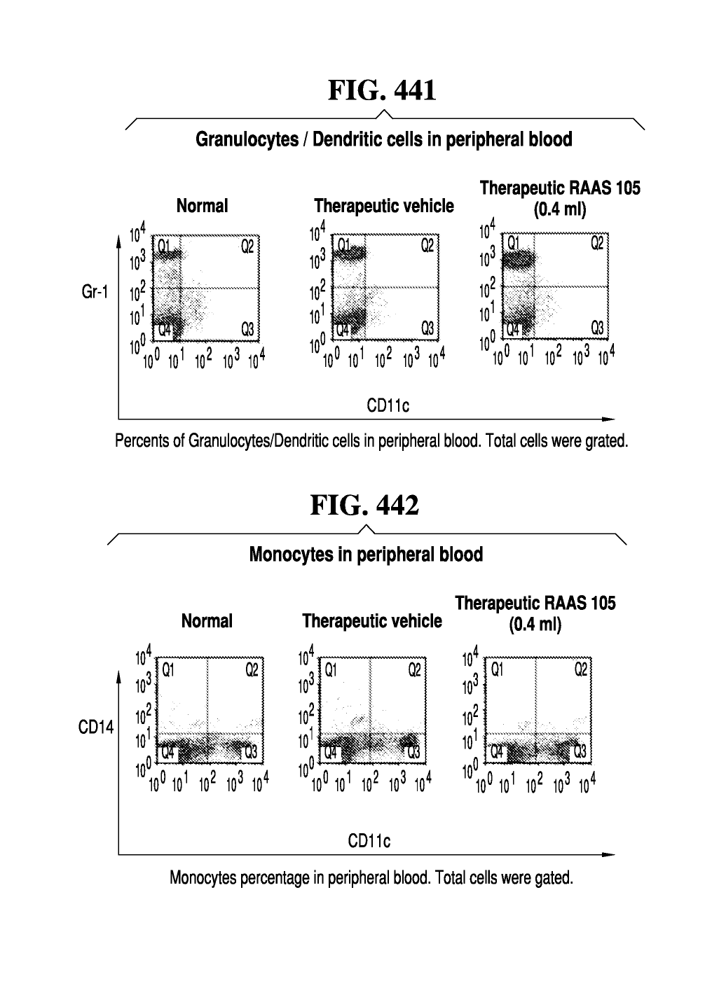

FIG. 441—Granulocytes/Dendritic cells in peripheral blood

Monocytes in peripheral blood is 52% lower in AFOD RAAS 105®

FIG. 442—Monocytes in peripheral blood

### Final Report of Efficacy Study on

### RAAS Antibodies in ApoE Mice

### Study Title: Efficacy Study of RAAS Antibodies on

### Atherosclerosis Model in ApoE Mice

### 1. Abbreviations and Definitions

kg kilogram
g gram
Mg milligram
ng Nano gram
ml Milliliter
μL microliter
h hours
min minutes
Cpd Compound
BW Body Weight
BG Blood Glucose
FBG Fasting Blood Glucose
DOB Date of Birth
TC Total Cholesterol
TG Triglyceride
LDL Low Density Lipoprotein
HDL High Density Lipoprotein
FBW Fasting Blood Glucose
SD Standard Deviation
SE Standard error
i.p Intraperitoneal injection
PFA paraformaldehyde

### 2. Introduction

The study described in this report evaluated in vivo efficacy of RAAS antibody APOA I on atherosclerotic model in ApoE knockout mice.

### 3. Purpose

To evaluate the efficacy effect of RAAS antibody APO AI on plasma lipid profile, plaque lesion of inner aorta and related parameters in atherosclerotic model.

### 4. Materials

4.1. Test article: RAAS Apo A I; Atorvastatin (reference compound)

4.2. Animal: ApoE knock out (ko) mouse

Sex: male

Strain: C57BL/6

Vender: Beijing Vitol River

Age: 8 weeks (arrived on 23 Dec. 2011)

Number: 60

4.3. Lipid profile test: Shanghai DaAn Medical Laboratory, Roche Modular automatic biochemistry analyzer

4.4. Heparin Sodium Salt: TCI, H0393

4.5. Capillary: 80 mm, 0.9-1.1 mm

4.6. Ophthalmic Tweezers and scissors: 66 vision-Tech Co., LTD, Suzhou, China. Cat#53324A, 54264TM

4.7. High Fat diet:TestDiet, Cat#58v8(35% kcal fat 1% chol)

4.8. Glycerol Jelly Mounting Medium: Beyotime, Cat# C0187.

4.9. Glucose test strips: ACCU-CHEK Performa: ROCHE (Lot#470396)

4.10. Image analyse: Aperio ScanScope system; Image-Proplus 6.0 software; Aperio image scope version 11.0.2.725 software.

4.11. Aorta staining: Oil Red O (Alfa Aesar) Isopropanol (Lab partner)

### 5. Experiment Method

### 5.1. Grouping Mice:

10 ApoE ko mice were fed with regular chow diet and used as negative control group. 50 ApoE ko mice were fed with high fat diet (35% kcal fat, 1% cholesterol) for 8 weeks, and then the plasma samples were collected for lipid profile measurement before the treatment. 50 ApoE ko mice were assigned into 5 groups based on the fasting overnight plasma TC and HDL level. The group information is shown in the table below.

TABLE 1
Information of groups

| | ApoE ko | | | Conc. Of | |
|---|---|---|---|---|---|
| Group | mice | Diet | Solution | CPD | Formulation |
| Negative Control | n = 10 | Normal diet | | | |
| Vehicles (saline) | n = 10 | High fat diet | 0.9% | | |
| | | | NaCL | | |
| ApoA1 High Dose: | n = 10 | High fat diet | | 5% Protein | |
| 0.1 ml i.p q.o d | | | | | |
| ApoA1 Mid Dose: | n = 10 | High fat diet | | 5% Protein | |
| 0.075 ml i.p q.o d | | | | | |
| ApoA1 Low Dose: | n = 10 | High fat diet | | 5% Protein | |
| 0.0.05 ml i.p q.o d | | | | | |
| Positive Control | n = 10 | High fat diet | 0.5% | 2 mg/mL | 20 mg + 10 ml |
| (Atorvastatin) 20 mg/kg | | | CMC | | 0.5% CMC |
| (increased to 40 mg/kg) | | | | | |

### 5.2. Study Timeline:

23 Dec. 2011: 60 ApoE mice arrived at chempartner and were housed in the animal facility in the building #3 for the acclimation.
6 Jan. 2012: Measured the body weight for each mouse. 50 mice were fed with high fat diet and 10 mice were fed with normal chow diet.
2 Mar. 2012: All mice were fasted over night and plasma samples (about 300 ul whole blood) were collected for lipid profile measurement before treatment with RAAS antibody.
19 Mar. 2012 to 6 Apr. 2012: Group the mice based on the TC and HDL level and start the treatment with 3 doses of antibody APOA1 by i.p daily on the weekday (The first dose was administered by iv injection via the tail vein. The reference compound atorvastatin was administered by oral dosing every day.
7 Apr. 2012 to 12 Apr. 2012: Stop dosing for 5 days. After 15 doses treatment with the antibody, several mice died in the treatment groups. The client asked for stopping treatment for a while.
13 Apr. 2012-6 Jul. 2012: The treatment with antibody APOA1 was changed to i.p injection every two days (Monday, Wednesday, and Friday) per client's instruction.
14 May 2012: All mice were fasted over night and plasma sample for each mouse (about 300 ul whole blood) was collected for lipid profile measurement after 8 weeks treatment.
9 Jul. 2012: All mice were fasted over night and plasma sample for each mouse (about 300 ul whole blood) was collected for lipid profile measurement after 16 weeks treatment. Blood glucose was also measured for each mouse.
9 Jul. 2012: The study was terminated after 16 weeks treatment. Measure BW, sacrificed each mouse, dissected the aorta, heart, liver and kidney and fixed them in 4% PFA.

### 5.3. Route of Compound Administration:

Antibody products were administrated by intraperitoneal injection every two days (Monday, Wednesday, and Friday). and the positive compound was administered by p.o every day.

### 5.4. Body Weight and Blood Glucose Measurement:

The body weight was weighed weekly during the period of treatment.

The fasting overnight blood glucose was measured at the end of study by Roche glucometer.

### 5.5 24 h Food Intake Measurement:

24 hours food intake for each cage was measured weekly

### 5.6. Plasma Lipid Profile Measurement:

About 300 ul of blood sample was collected from the orbital vein for each mouse and centrifuged at 7000 rpm for 5 min at 4° C. and the plasma lipid profile was measured by Roche Modular automatic biochemistry analyzer in DaAn Medical Laboratory

### 5.7. Study Taken Down:

After RAAS antibody products treatment for 16 weeks, all mice were sacrificed. Measured body weight and collected blood sample for each mouse. Weighed liver weight and saved a tiny piece of liver into 4% paraformaldehyde (PFA) fixation solution for further analysis. At same time, take the photos with heart, lung, aortas and two kidneys.

### 5.8. Oil Red Staining Procedure:

1. Sacrificed the mice and dissected the heart, aorta, and arteries under dissecting microscope.

2. Briefly wash with PBS and fixed in 4% paraformaldehyde (PFA) overnight at 4° C.

3. Rinse with 60% isopropanol

4. Stain with freshly prepared Oil Red O working solution 10 min.
1). Oil red O stock stain: 0.5% powder in isopropanol
2). Working solution: dilute with distilled water (3:2) and filter with membrane (0.22 um)

5. Rinse with 60% isopropanol 10 second.

6. Dispel the adherent bit fat outside of the aorta under the dissecting microscope.

7. Cut the vascular wall gently and keep the integrated arteries using the micro scissors.

8. Unfold the vascular inner wall with the cover slides and fix it by water sealing tablet.
5.9. Image Scanning and Analysis:

Scanning the glasses slides with the Aperio ScanScope system and analyze with the image proplus software to measure the area of atherosclerotic plaque lession. The results were expressed as the percentage of the total aortic surface area covered by lesions. The operation procedure of software was briefly described as follow: Converted the sys version photos into JPG version, then calibrated it and subsequently selected the red regions and then calculate the total area automatically by image proplus software.

### 5.10. Clinic Observation:

The information of dead animals was shown in the table as below.

TABLE 2
The information of dead and wounded mice

| | Animal Dead | | | | | Animal Wounded | |
|---|---|---|---|---|---|---|---|
| Group | Total | Cage No: | Animal No: | date | Reason | Total | Reason |
| Negative | 0 | | | | | 0 | |
| control | | | | | | | |
| Vehicle | 1 | 22 | 107 | 7 May | gastrorrhagia | 2 | fighting |
| Saline | | | | 2012 | and urinary | | each other |
| | | | | | tract infection | | |
| APOA 1 | 1 | 25 | 122 | 3 May | Fighting | 1 | fighting |
| high dose | | | | 2012 | | | each other |
| APOA 1 | 1 | 9 | 42 | 15 Apr. | Fighting | 1 | fighting |
| mid dose | | | | 2012 | | | each other |
| APOA 1 | 3 | 22 | 110 | 11 Apr. | gastrorrhagia | 3 | fighting |
| low dose | | | | 2012 | | | each other |
| | | 5 | 23 | 6 Jun. | urinary tract | | fighting |
| | | | | 2012 | infection | | each other |
| | | 25 | 125 | 7 Jun. | urinary tract | | fighting |
| | | | | 2012 | infection | | each other |
| Positive | 1 | 26 | 128 | 24 Mar. | enterorrhagia | 1 | fighting |
| control | | | | 2012 | | | each other |

### 6. Data Analysis

The results were expressed as the Mean±SEM and statistically evaluated by student's t-test. Differences were considered statistically significant if the P value was <0.05 or <0.01.

### 7. Results

### 7.1. Effect of APOA 1 on Body Weight

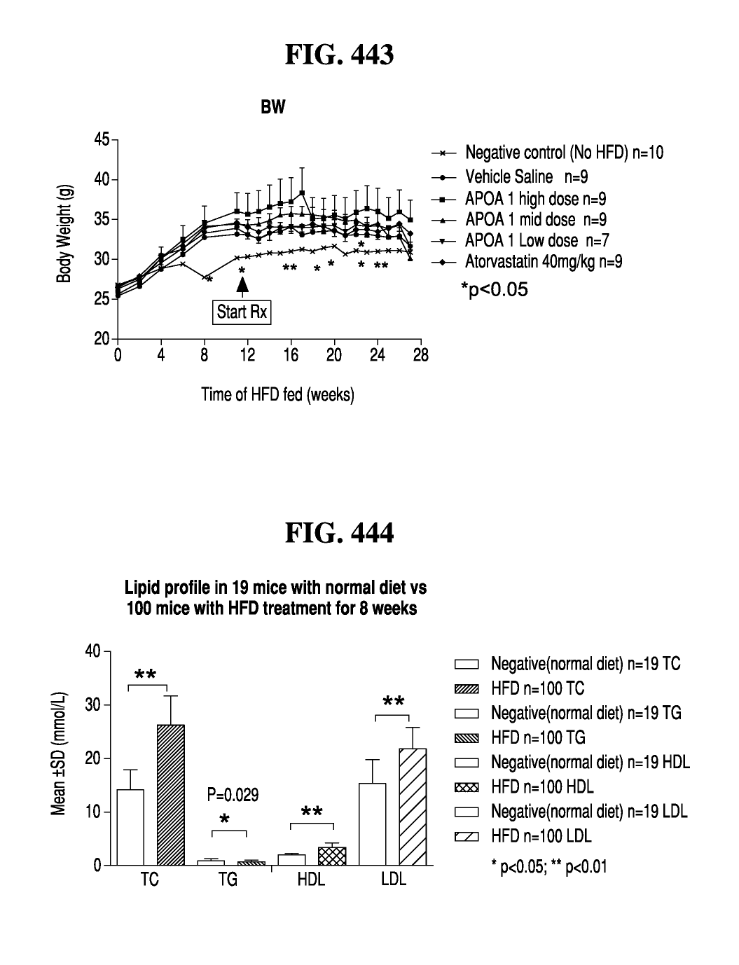

FIG. 443—Effect of APOA1 on body weight

The body weight in Apo E knockout mice fed with HFD significantly increased after 6 weeks treatment compared with the mice in negative control group that were fed with normal diet. There is no significant difference between the treatment groups and vehicle group.

### 7.2. Effect of HFD on Lipid Profile in ApoE Ko Mice

FIG. 444—Plasma lipid profile of ApoE mice fed with a normal diet and high fat diet

The lipid profile was measured in Apo E ko mice fed with high fat diet for 8 weeks. As shown above, plasma TC, TG, LDL as well as HDL in Apo E ko mice fed with high fat/high cholesterol for 8 weeks were significantly increased compared to Apo E KO mice fed with normal chow diet.

### 7.3. Effect of RAAS Antibody on Plasma Total Cholesterol (TC)

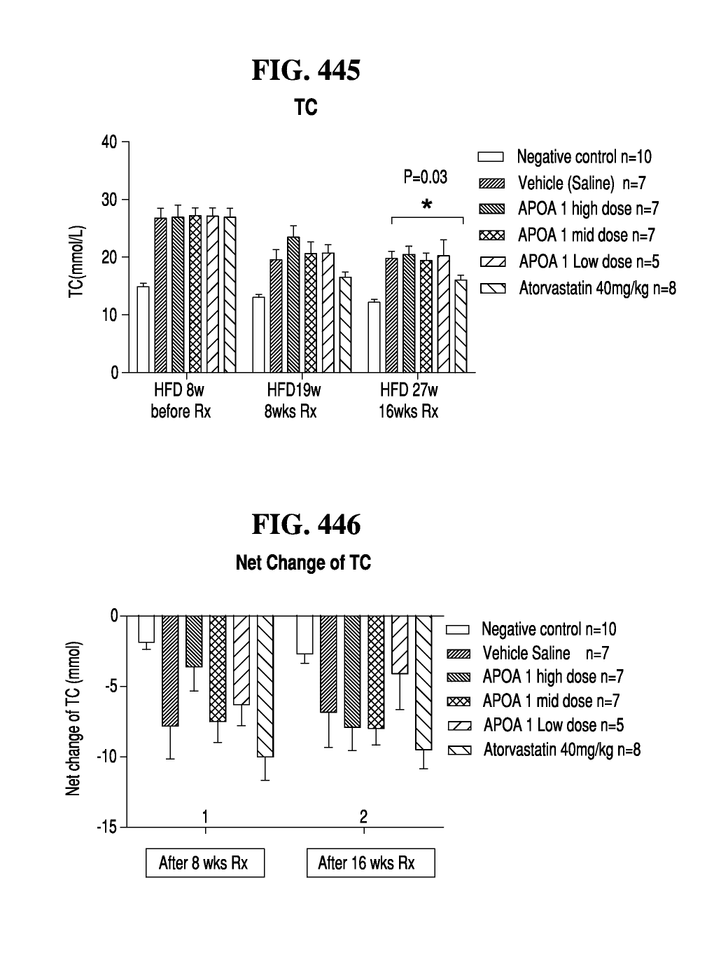

FIG. 445—Effect of RAAS antibody on plasma total cholesterol.

FIG. 446—Net change of RAAS antibody on plasma total cholesterol

As shown in the figure above, positive control atorvastatin can significantly lower total cholesterol level after 16 week treatment in ApoE ko mice but not reduce the TC net change.

### 7.4. The Effect of RAAS Antibody on Plasma Triglyceride (TG)

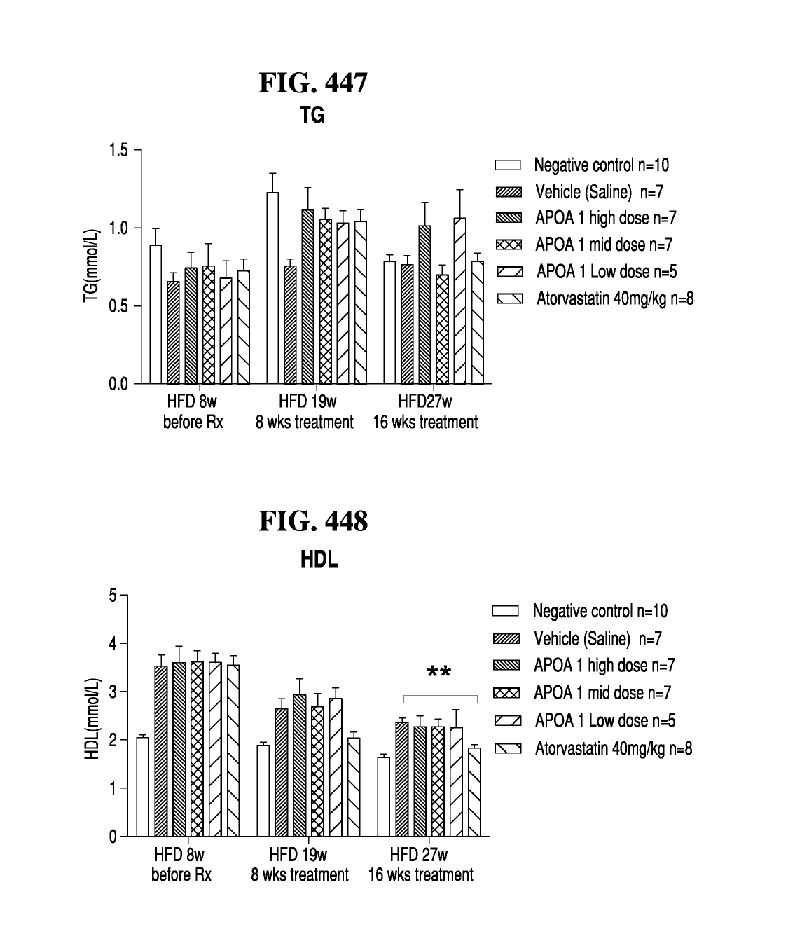

FIG. 447—The effect of RAAS antibody on total plasma Triglyceride

As shown in figure above, positive control atorvastatin and RAAS antibody had no effect on plasma TG level in Apo E ko mice fed with HFD after 16 weeks treatment.

### 7.5. The Effect of RAAS Antibody on High Density Lipoprotein (HDL)

FIG. 448—The effect of RAAS antibody on High Density Lipoprotein

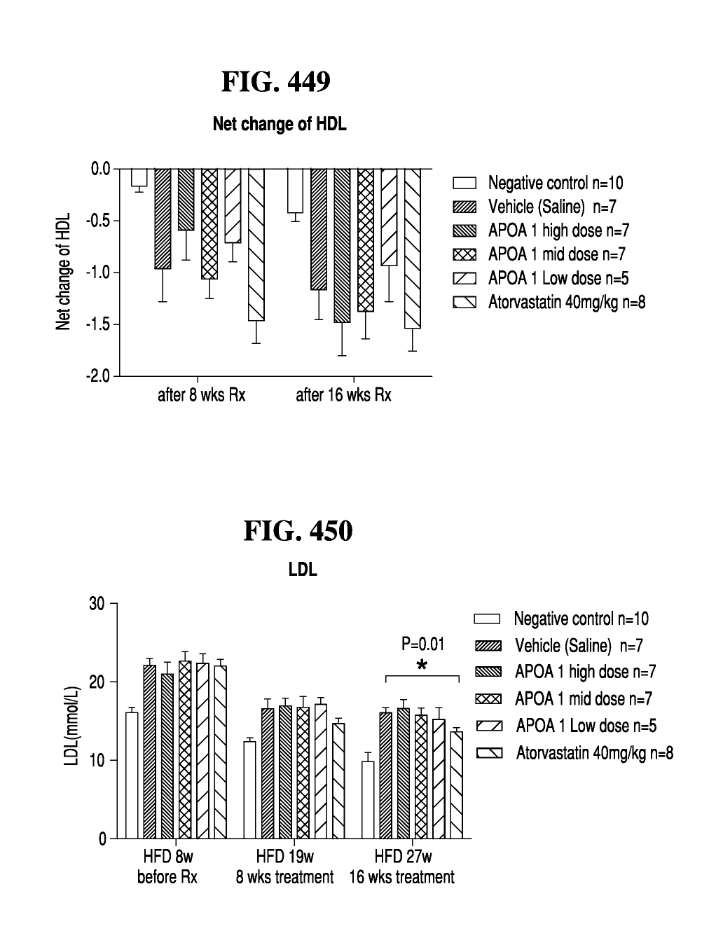

FIG. 449—Net change of RAAS antibody on High Density Lipoprotein

As shown in figure above, positive control atorvastatin can significantly lower high density lipoprotein in Apo E ko mice fed with HFD after 16 week treatment and RAAS antibody had a mild trend to decrease the HDL level in ApoE ko mice after 16 weeks treatment.

### 7.6. The Effect of RAAS Antibody on Low Density Lipoprotein (LDL)

FIG. 450—The effect of RAAS antibody on Low Density Lipoprotein

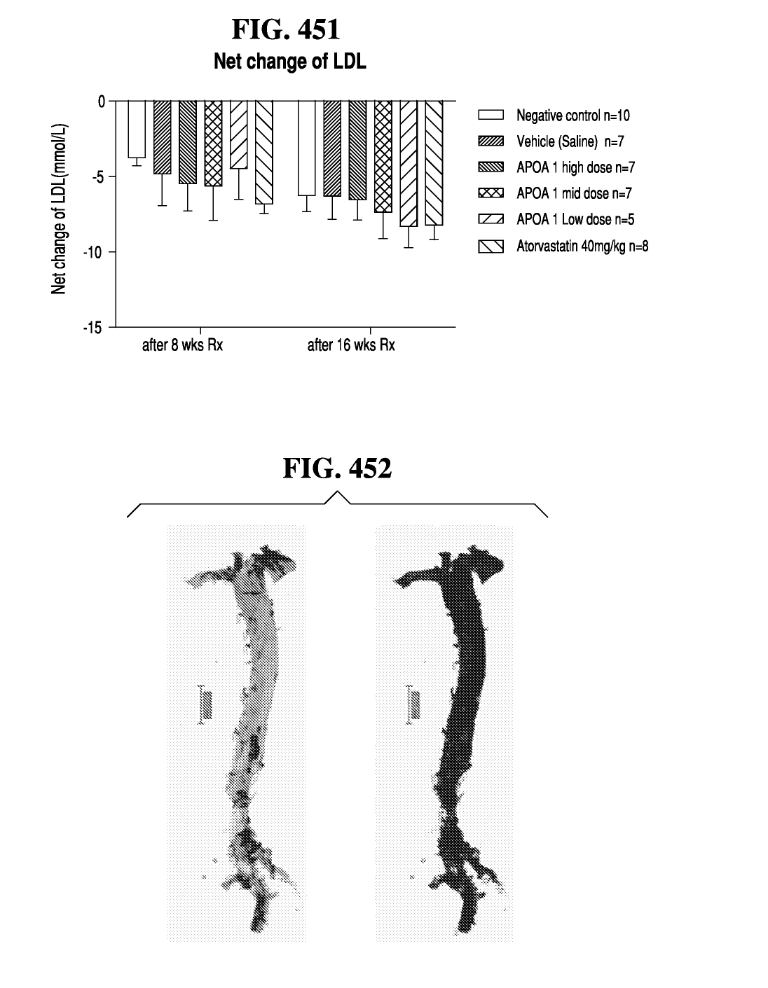

FIG. 451—Net change of RAAS antibody on Low Density Lipoprotein

As shown in figure above, positive control atorvastatin can significantly decrease low density lipoprotein in Apo E ko mice fed with HFD after 16 week treatment and there is no significant difference in net change of LDL.

### 7.7. The Effect of RAAS Antibody on Atherosclerosis Plaque Lesion

FIG. 452—Effect of RAAS antibody on negative control group on Atherosclerosis plaque lesion

As shown in the above diagram, we calculated all the plaque area stained by oil red and divided by total inner vascular area

Area Percent (%)=Sum Area of Atherosclerotic Plaque (Mm²)/Whole Area of Vascular Inner Wall (Mm²)

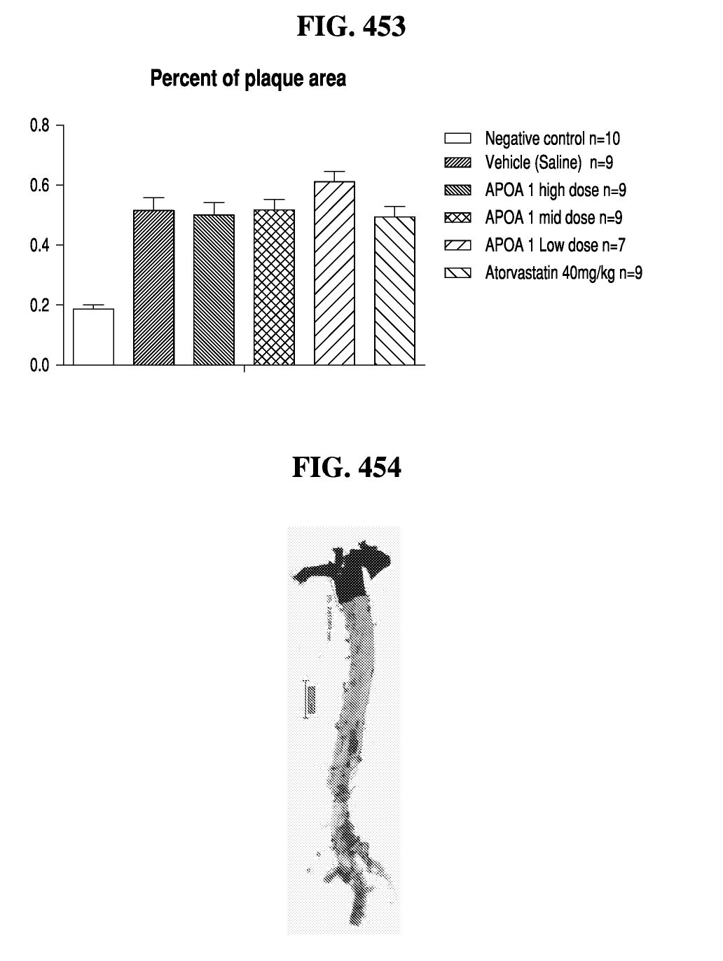

FIG. 453—Percent of plaque area in total inner vascular area

No significant difference between the vehicle and treatment groups in plaque area and percentage of plaque area although Atorvastatin showed a mild trend to decrease percentage of plaque area after 16 weeks oral administration.

FIG. 454—Illustrated analysis of arterial arch area

The total area of aorta from the aortic root to the thoracic aorta was measured (bracketed area).

As shown in the left panel, because the total lumen area in arterial arch is very difficult to identify in en face vessel, we measured the total area at the length of about 2 mm from aortic root down to the thoracic artery (bracketed area).

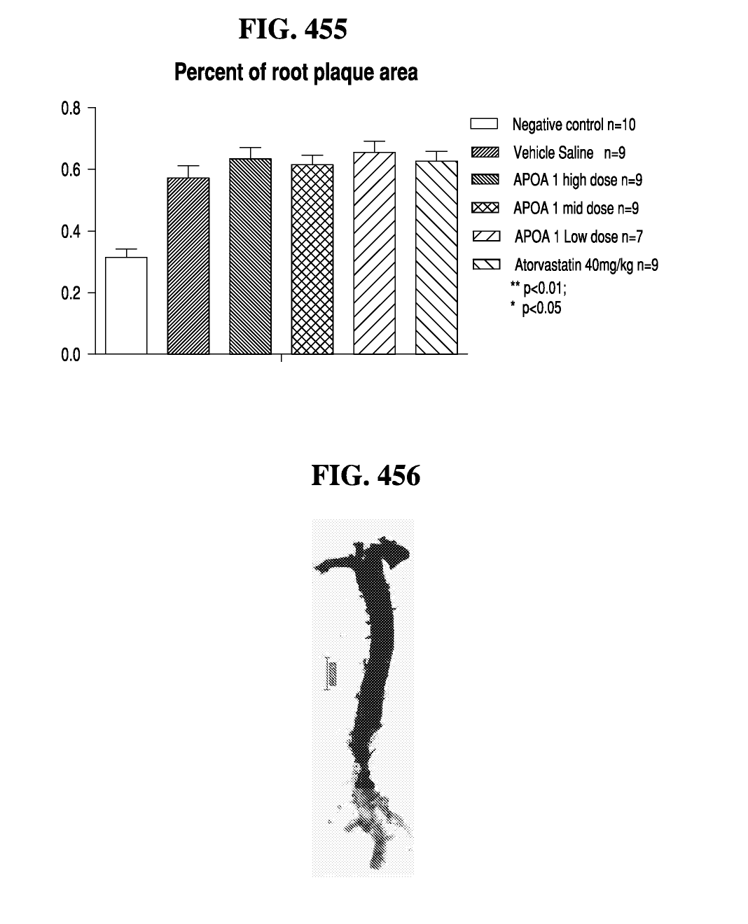

FIG. 455—Percent of plaque area in the arterial arch area

The plaque lesion was more severe in mice fed with HFD than mice in the normal diet (negative) group. No significant difference between the vehicle and treatment groups in plaque area and percentage of plaque area.

FIG. 456—Illustrated analysis from root to right renal artery

As shown in the left panel, the total area from the aortic root to the right renal artery were measured (bracketed area)

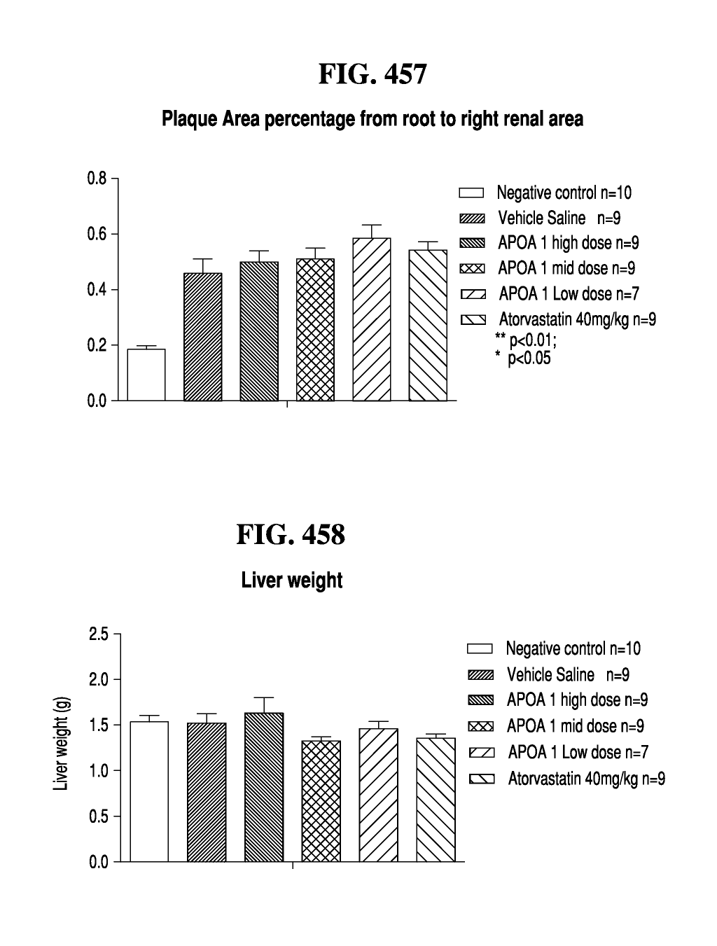

FIG. 457—Percent of plaque area from root to right renal artery

There is no significant difference between vehicle and treatment groups in plaque area and percentage of plaque area.

### 7.8. The Effect of RAAS Antibody on Liver Weight

FIG. 458—Diagram of liver weight

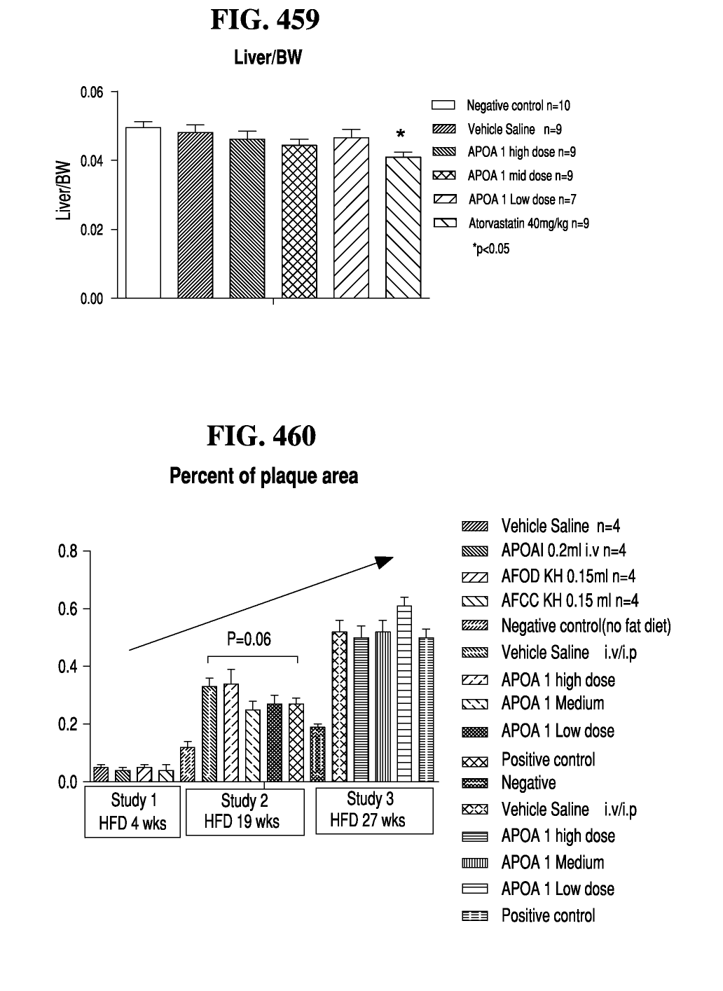

FIG. 459—Diagram of liver index

Atorvastatin at 20 mg/kg reduced the ratio of liver/body weight significantly after 16 weeks treatment, which is consistent with the 8 weeks treatment result in study 2.

### 7.9 Comparison of Percentage of Plaque Area in Study 1, 2, 3

FIG. 460—Comparison of percentage of plaque area in study 1, 2, 3

We also compared percent of plaque area in the study 1, 2 and 3. In study 1, all ApoE ko mice were fed with HFD for 4 weeks and mice were sacrificed at 14 weeks of age. In study 2, all ApoE ko mice were fed with HFD for 19 weeks except the mice in negative control group and all mice were sacrificed at 29 weeks of age. In study 3, the ApoE ko mice were fed with HFD for 27 weeks and sacrificed at 37 weeks. It is apparent that:

1. The plaque area increased steadily with HFD feeding time or aging.

2. The aorta atherosclerosis model in ApoE ko mouse was established successfully.

3. HFD feeding for 10 weeks plus 8 weeks Rx gave best result.

### 7.10 Comparison of TC Level in Study 1, 2, 3

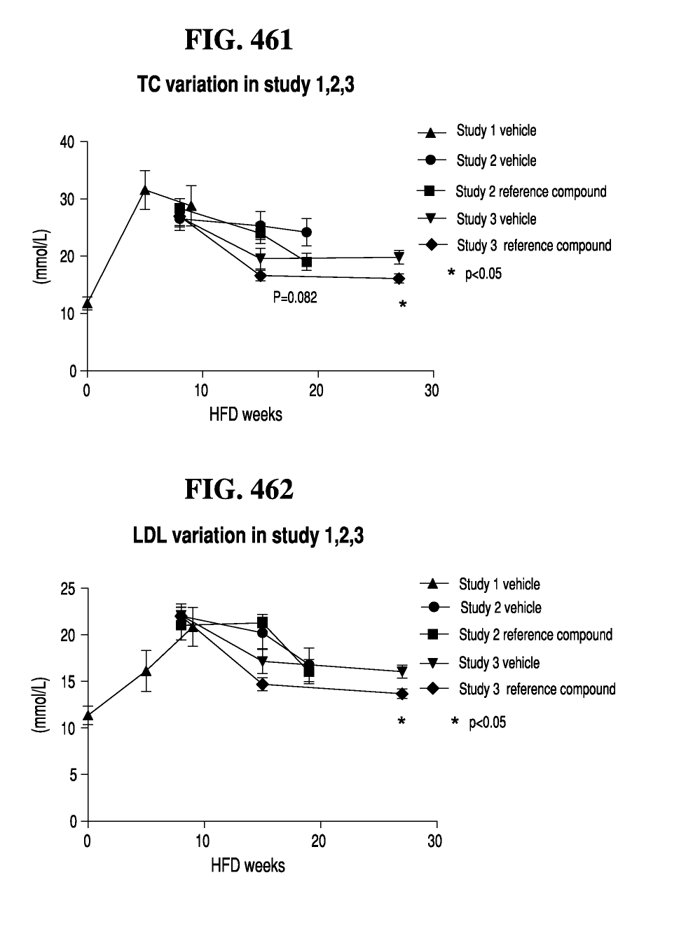

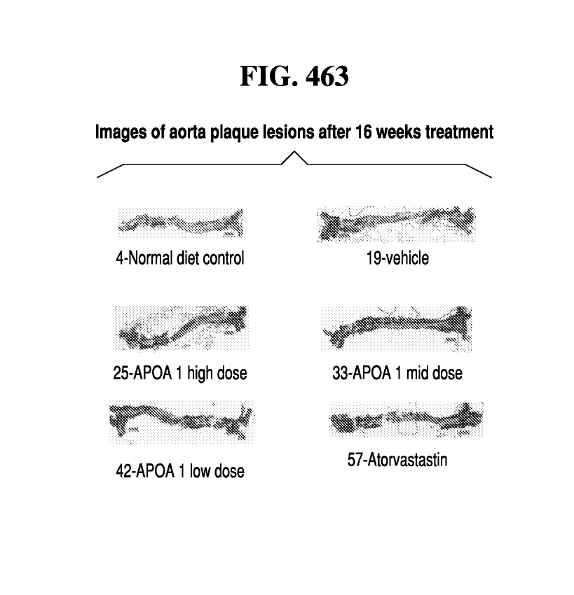

FIG. 461—Comparison of Total Cholesterol level in study 1, 2, 3

FIG. 462—Comparison of percentage of plaque area in study 1, 2, 3

The TC and LDL values from study 1, 2 and 3 in vehicle and reference groups peaked at week 10, and deceased subsequently during 27 weeks high fat diet feeding. This phenomenon was also observed in relevant literature reports (details can be seen in the report on ppt. version).

7.11. Image of Aorta with Red Oil Staining

One image of aorta stained by oil red from each group was selected and showed below. The branches of artery and the lipid plaques could be observed clearly and the plaques mainly distribute in the aortic root and principal branches of the abdominal aorta. It is consistent with the reference literatures.

### 8. Summary and Interpretation

1). Atorvastatin at 40 mg/kg significantly reduced liver/BW ratio, plasma TC, HDL and LDL, but did not affect the plaque lesion area of aorta in ApoE KO mice after 16 weeks treatment.
2). RAAS APOA1 did not affect the lipid profile in ApoE KO mice after 16 weeks treatment.
3). RAAS APOA1 did not reduce the plaque lesion area of aorta in ApoE KO mice after 16 weeks treatment.

### Interpretation:

1). The % athero-plaque lesion area reached 50% at the end of 16 week treatment. The 26 week HFD feeding might have made the mice too sick for the test drugs to reverse.
2). Seems 8 weeks treatment gave optimal athero-plaque reduction, as shown by RAAS Study 2 as well as by literature reports.
3). If repeat, suggest to reduce the HFD feeding duration before drug treatment to <6 weeks, and keep the treatment duration to 8 weeks.

### 9. Conclusion:

1). Atorvastatin at 40 mg/kg significantly reduced plasma TC, HDL and LDL level, liver weight and the ratio of liver/BW, but did not affect the plaque lesion area of aorta in ApoE KO mice after 16 weeks treatment.
2). RAAS antibody APOA1 didn't affect the lipid profile and reduce the plaque lesion of aorta in ApoE KO mice after 16 weeks treatment.

ApoE KO mice which lack the ApoE gene, therefore the RNA synthesized a bad protein that concluded in a controversial result. As the LDL is higher than the HDL, while 98% of the tested mediums have a higher HDL than LDL.

In the first four weeks and then 8 weeks, it had been proven that a certain percentage of the plaque had been removed by AFOD RAAS 1® however on the 16^{th} week it showed no further effect as the gene of the ApoE KO mice is already modified so the RNA cannot send the signal to synthesize the good proteins. While in the rabbit study with no modified gene has shown good results of removing the plaque up to 40%.

### Conclusion:

The inventor has determined that cells from any source never die.

Regardless from any source, animal, plant, fruit, human all cells are the same. The structure of all cells have the DNA and RNA. The function of any cell are the same as the function of the human cells, including KH good healthy cells in which the RNA synthesizes good proteins that:

1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals.

Because the of the above mechanism of the good healthy KH cells they can CURE, PROTECT, and PREVENT diseases, viruses infections, bacteria infections, auto immune disease, neurological disorder, all type of solid and blood cancer, coagulation, diabetic, inhibitor, immune deficiency, muscle and nerve repair and restoration, infiltration of radiation or any pollutant.

An animal good healthy cell has the same mechanism and function as the above mentioned good healthy KH cells. This can help cure diseases like H1N1, H5N1, mad cow disease, Foot and Mouth disease, blue ear disease in chicken, cow and pig and infiltration of radiation or any pollutant.

A Plant good healthy cell has the same mechanism and function as the above mentioned good healthy KH cells. This will help protect the crops from diseases and infiltration of radiation or any pollutant.

Like calories in a meal, the inventor believes that we can calculate to have enough good proteins by the UNITS of the cell. For example, in 20 microliters of KH101 (non-sticky rice) the inventor found 20 million cells.

In order to have the best diet to prevent the diseases, infection or disorders we can select the cell that synthesizes the good protein like lettuce, carrot, cucumber, egg white, etc instead of the one that contains the bad proteins like giant clam, fat from beef, chicken or pork, etc. which is harder for the body system to digest.

In conclusion protein is fat which can be found in the protein from plasma derived medicine products, recombinant DNA products, Monoclonal products, Animal derived or plant derived.

Thanks to the detection of the lipid panel in each particular product we can avoid the product for consumption which contains a lot of bad FAT.

Under a microscope you cannot tell the difference between a good healthy KH cell and a bad, damaged and sick cell as the RNA is the key element which synthesizes a good healthy protein or a bad, damaged and sick protein.

The good KH healthy cells

A good protein in which the KH good healthy cell whose RNA exists: 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals.

The bad, damaged and sick cells.

A bad, damaged or sick cell whose RNA synthesize a bad protein to cause the deficiency inhibitor disease and cancers.

Any protein in which the cell exists and has been modified has become a bad, damaged cell as the gene has been altered. This has been proven in genetically modified rice, genetically modified corn, E. coli and genetically modified cell in HEK293 which is a human cell.

The inventor concludes that any animal, human being, plant or any organism that has cells whose gene has been modified is no longer a GOOD HEALTHY KH CELLS and has become a BAD, DAMAGED AND SICK cells like in the case of the APOE knock out mice in which we found the LDL and triglycerides are much higher than the HDL. In 4 weeks to 8 weeks the plaque removal has been 30% to 40% reduction however in 16 weeks has no effect at all.

THE GENE THERAPY WILL NOT WORK. This has been proven by the following articles extracted from the internet:

http://www.bionews.org.uk/page_{—}12237.asp

A gene therapy trial for an inherited immune deficiency disorder has been suspended again, following the appearance of complications in a third child. Eleven patients affected by X-linked severe combined immunodeficiency disorder (X-SCID) have so far been treated by the team, based at the Necker Hospital in Paris. While most have responded extremely well to the therapy, the trial was suspended in late 2002, after two patients developed symptoms of leukemia. One of these boys is now in remission, but the other has since died. The AFSSAPS (French Agency for Health Product Safety) gave permission for the trial to restart in May 2004, but has now suspended the experimental treatment again.

Children affected by SCID have a faulty gene that means they have no working immune system, so their bodies cannot fight infections. This life-threatening condition is sometimes called ‘bubble boy’ disease, as unless they can be successfully treated with a matched bone marrow transplant, patients must spend their lives in a sterile environment. To carry out the gene therapy treatment, the French researchers harvested bone marrow from the patients, from which they isolated blood stem cells. They then infected these cells with a retrovirus (a virus that inserts its genetic material into the host cell's DNA) carrying a working gene, before returning the modified cells back to the patients.

Scientists think that the leukemia in the two patients reported in 2002 was caused by the gene therapy, although other factors may also have contributed. In both cases, researchers found that the retrovirus had inserted its genetic material close to the ‘on-switch’ of a cancer-causing gene called LMO2. It is thought that this event caused the unregulated growth of the bone marrow cells, which in turn triggered the leukemia. Now, a patient who was treated in April 2002, at the age of nine months, is also showing signs of ‘lymphoproliferation’—overgrowth of white blood cells. The AFSSAPS has suspended the trial while the causes of this latest complication are investigated, French newspapers reported last week.

http://www.bioresearchonline.com/doc.mvc/Patient-Death-Puts-Pall-Over-Gene-Therapy-0001

Gene therapy may come under closer scrutiny following the death of a teen-ager during an experiment at the University of Pennsylvania.

The “Washington Post” reports that this is the first death attributed to genetic research. Scientists say Jesse Gelsinger, 18 of Tucson, Ariz. fell ill and died four days after doctors infused his liver with genetically-engineered viruses. The gene therapy experiment has now been halted.

1. A method of introduction of good healthy cells selected from the group consisting of DRAGON CELLS, SNAKE CELLS, GOOD HEALTHY DOUBLE RINGS DIFFERENT SIZE CELLs, GOOD HEALTHY LIGHTNING CELLs, GOOD HEALTHY SQUARE PIXEL CELLs, BEAMING RAYS CELLs, GOOD HEALTHY RECONSTRUCTION BACKGROUND CELLs, GOOD HEALTHY FACET CELLs, GOOD HEALTHY CRATER CELLs, GOOD HEALTHY YELLOW CELLs, GOOD HEALTHY LEER CELLs, containing Good Proteins to send signal to DNA of sick, bad, damaged cells to transform RNA to synthesize Good Protein to cure diseases, viruses infections, Bacteria Infection, Auto immune disease, Neurological disorder, all type of 150 solid and blood cancers, coagulation, Diabetic, Inhibitor, Immune deficiency.

2. The method of claim 1, wherein the good healthy cells are SNAKE CELLs.

3. The method of claim 1, wherein the good healthy cells are GOOD HEALTHY DOUBLE RINGS DIFFERENT SIZE CELLs.

4. The method of claim 1, wherein the good healthy cells are GOOD HEALTHY LIGHTNING CELLs.

5. The method of claim 1, wherein the good healthy cells are GOOD HEALTHY SQUARE PIXEL CELLs.

6. The method of claim 1, wherein the good healthy cells are BEAMING RAYS CELLs.

7. The method of claim 1, wherein the good healthy cells are GOOD HEALTHY RECONSTRUCTION BACKGROUND CELLs.

8. The method of claim 1, wherein the good healthy cells are GOOD HEALTHY FACET CELLs.

9. The method of claim 1, wherein the good healthy cells are GOOD HEALTHY CRATER CELLs.

10. The method of claim 1, wherein the good healthy cells are GOOD HEALTHY YELLOW CELLs.

11. The method of claim 1, wherein the good healthy cells are GOOD HEALTHY LEER CELLs.

12. The method of claim 1, wherein the good proteins comprise C3 Complement C3.

13. The method of claim 1, wherein the good proteins comprise Good Protein ENO1 Isoform.

14. The method of claim 1, wherein the good proteins comprise Good Protein TUFM elongation factor.

15. The method of claim 1, wherein the good proteins comprise Good Protein ASS1 Argininosuccinate.

16. The method of claim 1, wherein the good proteins comprise Good Protein ANXA2 Isoform 2 of Annexin A2.

17. The method of claim 1, wherein the good proteins comprise Good Protein Glyceraldehyde-3-phosphate dehydrogenase.

18. The method of claim 1, wherein the good proteins comprise Good Protein KRT 86 Keratin.

19. The method of claim 1, wherein the good proteins comprise Good Protein type II cuticular HB6.

20. The method of claim 1, wherein the good proteins comprise Good Protein LDHA Isoform 1 of L-lactate dehydrogenase A chain.

21. The method of claim 1, wherein the good proteins comprise Good Protein Fibrin beta.

22. The method of claim 1, wherein the good proteins comprise Good Protein Growth-inhibiting protein 25.

23. The method of claim 1, wherein the good proteins comprise Good Protein Fibrinogen gama.

24. The method of claim 1, wherein the good proteins comprise Good Protein Chain L, Crystal structure of Human Fibrinogen.

25. The method of claim 1, wherein the good proteins comprise Good Protein Chain A of IgM.

26. The method of claim 1, wherein the good proteins comprise Good Protein Chain A Crystal structure of the Fab fragment of A Human Monoclonal Igm Cold Agglutinin.

27. The method of claim 1, wherein the good proteins comprise Good Protein Immunoglobulin light chain.

28. The method of claim 1, wherein the good proteins comprise Good Protein Chain C, Molecular Basis for Complement Recognition.

29. The method of claim 1, wherein the good proteins comprise Good Protein CP 98 kDa protein.

30. The method of claim 1, wherein the good proteins comprise Good Protein CP Reuloplasmin.

31. The method of claim 1, wherein the good proteins comprise Good Protein KRT2 Keratin, type II cytoskeletal epidermal.

32. The method of claim 1, wherein the good proteins comprise Good Protein APOA1 Apolipoprotein A-1.

33. The method of claim 1, wherein the good proteins comprise Good Protein Human Albumin.

34. The method of claim 1, wherein the good proteins comprise Good Protein Transferrin.

35. The method of claim 1, wherein the good proteins comprise Good Protein Vimentin.

36. The method of claim 1, wherein the good proteins comprise Good Protein Haptoglobin.

37. The method of claim 1, wherein the good proteins comprise Good Protein KH1.

38. The method of claim 1, wherein the good proteins comprise Good Protein KH2.

39. The method of claim 1, wherein the good proteins comprise Good Protein KH3.

40. The method of claim 1, wherein the good proteins comprise Good Protein KH4.

41. The method of claim 1, wherein the good proteins comprise Good Protein KH5.

42. The method of claim 1, wherein the good proteins comprise Good Protein KH6.

43. The method of claim 1, wherein the good proteins comprise Good Protein KH7.

44. The method of claim 1, wherein the good proteins comprise Good Protein KH8.

45. The method of claim 1, wherein the good proteins comprise Good Protein KH9.

46. The method of claim 1, wherein the good proteins comprise Good Protein KH10.

47. The method of claim 1, wherein the good proteins comprise Good Protein KH11.

48. The method of claim 1, wherein the good proteins comprise Good Protein KH12.

49. The method of claim 1, wherein the good proteins comprise Good Protein KH13.

50. The method of claim 1, wherein the good proteins comprise Good Protein KH14.

51. The method of claim 1, wherein the good proteins comprise Good Protein KH15.

52. The method of claim 1, wherein the good proteins comprise Good Protein KH16.

53. The method of claim 1, wherein the good proteins comprise Good Protein KH17.

54. The method of claim 1, wherein the good proteins comprise Good Protein KH18.

55. The method of claim 1, wherein the good proteins comprise Good Protein KH19.

56. The method of claim 1, wherein the good proteins comprise Good Protein KH20.

57. The method of claim 1, wherein the good proteins comprise Good Protein KH21.

58. The method of claim 1, wherein the good proteins comprise Good Protein KH22.

59. The method of claim 1, wherein the good proteins comprise Good Protein KH23.

60. The method of claim 1, wherein the good proteins comprise Good Protein KH24.

61. The method of claim 1, wherein the good proteins comprise Good Protein KH25.

62. The method of claim 1, wherein the good proteins comprise Good Protein KH26.

63. The method of claim 1, wherein the good proteins comprise Good Protein KH27.

64. The method of claim 1, wherein the good proteins comprise Good Protein KH28.

65. The method of claim 1, wherein the good healthy cells are dragon cells.

66. A method of introduction of any combination of any one or as many GOOD HEALTHY CELLs from any source discovered in this patent application or not yet discovered containing Good Proteins to send signal to DNA of sick, bad, damaged cells to transform RNA to synthesize Good Protein to cure diseases, viruses infections, Bacteria Infection, Auto immune disease, Neurological disorder, all type of 150 solid and blood cancers, coagulation, Diabetic, Inhibitor, Immune deficiency or by any means these GOOD HEALTHY CELLs are found to be effective against the diseases, viruses infections, Bacteria, Infection, Auto immune disease, Neurological disorder, all type of 150 solid and blood cancers, coagulation, Diabetic, Inhibitor, Immune deficiency.

67. The GOOD HEALTHY CELLs not only as described 11 types of cells above but also composed of any shape of the cell which have been identified in this patent application (FIG. 1 through FIG. 23) or not yet been identified that can send signal to DNA of sick, bad, damaged cells to transform RNA to synthesize Good Proteins to cure diseases, viruses infections, Bacteria Infection, Auto immune disease, Neurological disorder, all type of 150 solid and blood cancers, coagulation, Diabetic, Inhibitor, Immune deficiency or by any means these GOOD HEALTHY CELLs are found to be effective against the diseases, viruses infections, Bacteria, Infection, Auto immune disease, Neurological disorder, all type of 150 solid and blood cancers, coagulation, Diabetic, Inhibitor, Immune deficiency.

68. The method of claim 1, wherein the size of all these GOOD HEALTHY CELLs containing good protein is no greater than 20 nanometers.

69. The method of claim 1, wherein the GOOD HEALTHY CELLs containing good proteins do not die or get damaged as long as they are in plasma, fraction of plasma or in final product.

70. The method of claim 1, wherein the GOOD HEALTHY CELLs containing good proteins do not die going through virus inactivation by the method of solvent detergent which is known for killing the HVB, HCV, HIV.

71. The method of claim 1, wherein the GOOD HEALTHY CELLs containing good proteins do not die when heat up to 60 degrees Celsius for 20 hours and 100 degrees Celsius for 30 minutes.

72. The method of claim 1, wherein the GOOD HEALTHY CELLs containing good proteins do not die when alcohol is added up to 40% and through high speed centrifugation.

73. The method of claim 1, wherein the GOOD HEALTHY CELLs containing good proteins do not die in freeze-drying process or liquid format.

74. The method of claim 1, wherein the GOOD HEALTHY CELLs containing good proteins cannot be stripped off during the ultra filtration using different sizes of filters from 0.45 micron to 0.2 micron and even 50 Nano meters to 20 Nano meters.

75. The method of claim 1, wherein the GOOD HEALTHY CELLs containing good proteins are living in the proteins of the final product both liquid and lyophilized format including the recombinant DNA and live up to 10 years or more.

76. The method of claim 1, wherein the GOOD HEALTHY CELLs containing good proteins are durable and resistant and never die during the process of fractionation, further purification, lyophilized, virus inactivation, and final are living in the final products.

77. The method of claim 1, wherein the GOOD HEALTHY CELLs containing good proteins are living and has never been killed and stripped off from the protein.

78. The method of claim 1, wherein the GOOD HEALTHY CELLs contain the Good Protein nitric oxide synthase 1 (neuronal), isoform CRA_b.

79. The method of claim 1, wherein the GOOD HEALTHY CELLs contain the GOOD HEALTHY CELLs containing Good Protein Chain L, Crystal Structure Of Human Fibrinogen.

80. The method of claim 1, wherein the GOOD HEALTHY CELLs contain the Good Protein Chain A, Structure Of Human Serum Albumin.

81. The method of claim 1, wherein the GOOD HEALTHY CELLs contain the Good Protein Chain A, Human Serum Albumin In A Complex With Myristic Acid And Tri-Iodobenzoic Acid.

82. The method of claim 1, wherein the GOOD HEALTHY CELLs contain the Good Protein Chain A, Structure Of Human Serum Albumin With S-Naproxen And The Ga Module.

83. The method of claim 1, wherein the GOOD HEALTHY CELLs contain the Good Protein Chain G, Crystal Structure Of Human Fibrinogen.

84. The method of claim 1, wherein the GOOD HEALTHY CELLs contain the Good Protein fibrin beta (in Cryoprecipitate). claim safe

84. The method of claim 1, wherein the GOOD HEALTHY CELLs contain the Good Protein fibrin gamma (in Cryoprecipitate).

85. The method of claim 1, wherein the GOOD HEALTHY CELLs contain the Good Protein Chain A, Crystal Structure Of A1pi-Pittsburgh In The Native Conformation.

86. The method of claim 1, wherein the GOOD HEALTHY CELLs contain the Good Protein Keratin, Type II cytoskeletal (in Cryoprecipitate).

87. The method of claim 1, wherein the GOOD HEALTHY CELLs contain the Good Protein vinculin, isoform CRA_a (in fraction III).

88. The method of claim 1, wherein the GOOD HEALTHY CELLs contain the Good Protein Chain A, Crystal Structure Of Complement C3b In Complex With Factors B And D (in fraction III).

89. The method of claim 1, wherein the GOOD HEALTHY CELLs contain the Good Protein fibrin beta (in fraction III).

90. The method of claim 1, wherein the GOOD HEALTHY CELLs contain the Good Protein Chain A, Human Serum Albumin In A Complex With Myristic Acid And Tri-Iodobenzoic Acid (in fraction III).

91. The method of claim 1, wherein the GOOD HEALTHY CELLs contain the Good Protein Chain I, P14-Fluorescein-N135q-5380c-Antithrombin-Iii (in fraction III).

92. The method of claim 1, wherein the GOOD HEALTHY CELLs contain the Good Protein growth-inhibiting protein 25 (in fraction III).

93. The method of claim 1, wherein the GOOD HEALTHY CELLs contain the Good Protein Chain L, Crystal Structure Of Human Fibrinogen (in fraction III).

94. The method of claim 1, wherein the GOOD HEALTHY CELLs contain the Good Protein fibrinogen gamma (in fraction III).

95. The method of claim 1, wherein the GOOD HEALTHY CELLs contain the Good Protein CD5 antigen-like (in fraction III).

96. The method of claim 1, wherein the GOOD HEALTHY CELLs contain the Good Protein apolipoprotein A-IV precursor (in fraction III).

97. The method of claim 1, wherein the GOOD HEALTHY CELLs contain the Good Protein Chain C, Molecular Basis For Complement Recognition (in fraction III).

97. The method of claim 1, wherein the GOOD HEALTHY CELLs contain the Good Protein Chain B, H-Ficolin (in fraction III).

98. The method of claim 1, wherein the GOOD HEALTHY CELLs contain the Good Protein complement C4-B-like isoform 2 (in fraction III).

99. The method of claim 1, wherein the GOOD HEALTHY CELLs contain the Good Protein immunoglobulin light chain (in fraction III).

100. The method of claim 1, wherein the GOOD HEALTHY CELLs contain the Good Protein Chain A, Crystal Structure Of The Fab Fragment Of A Human Monoclonal Igm Cold Agglutinin (in fraction III).

101. The method of claim 1, wherein the GOOD HEALTHY CELLs contain the Good Protein PR domain containing 8, isoform CRA_b (in fraction III).

102. The method of claim 1, wherein the GOOD HEALTHY CELLs contain the Good Protein Chain D, The Structure Of Serum Amyloid P Component Bound To Phosphoethanolamine (in fraction III).

103. The method of claim 1, wherein the GOOD HEALTHY CELLs contain the Good Protein retinol binding protein 4, plasma, isoform CRA_a (Prothrombin Complex Concentrate).

104. The method of claim 1, wherein the GOOD HEALTHY CELLs contain the Good Protein Chain A, Crystal Structure Of Transthyretin In Complex With Iododiflunisal-Betaalaoh (Prothrombin Complex Concentrate).

105. The method of claim 1, wherein the GOOD HEALTHY CELLs contain the Good Protein complement component 9, isoform CRA_a (Prothrombin Complex Concentrate).

106. The method of claim 1, wherein the GOOD HEALTHY CELLs contain the Good Protein kininogen 1, isoform CRA_a (Prothrombin Complex Concentrate).

107. The method of claim 1, wherein the GOOD HEALTHY CELLs contain the Good Protein beta-tubulin (Prothrombin Complex Concentrate).

108. The method of claim 1, wherein the GOOD HEALTHY CELLs contain the Good Protein vimentin, isoform CRA_a (Prothrombin Complex Concentrate).

109. The method of claim 1, wherein the GOOD HEALTHY CELLs contain the Good Protein complement component C4B (Prothrombin Complex Concentrate).

110. The method of claim 1, wherein the GOOD HEALTHY CELLs contain the Good Protein Chain C, Molecular Basis For Complement Recognition And Inhibition Determined By Crystallographic Studies Of The Staphylococcal Complement Inhibitor (Scin) (Prothrombin Complex Concentrate).

111. The method of claim 1, wherein the GOOD HEALTHY CELLs contain the Good Protein Bound To C3c And C3 (Prothrombin Complex Concentrate).

112. The method of claim 1, wherein the GOOD HEALTHY CELLs contain the Good Protein Chain D, The Structure Of Serum Amyloid P Component Bound To Phosphoethanolamine (Prothrombin Complex Concentrate).

113. The method of claim 1, wherein the GOOD HEALTHY CELLs contain the Good Protein 4-kDa subunit of Complex I (Prothrombin Complex Concentrate).

114. The method of claim 1, wherein the GOOD HEALTHY CELLs contain the Good Protein A1AT (Fraction paste IV).

115. The method of claim 1, wherein the GOOD HEALTHY CELLs contain the Good Protein vitamin D-binding protein precursor (Fraction paste IV).

116. The method of claim 1, wherein the GOOD HEALTHY CELLs contain the Good Protein Semenogelin-1 (Fraction paste IV).

117. The method of claim 1, wherein the GOOD HEALTHY CELLs contain the Good Protein Haptoglobin (Fraction paste IV).

118. The method of claim 1, wherein the GOOD HEALTHY CELLs contain the Good Protein Vimentin (Fraction paste IV).

119. The method of claim 1, wherein the GOOD HEALTHY CELLs contain the Good Protein Nesprin-2 (Fraction paste IV).

120. The method of claim 1, wherein the GOOD HEALTHY CELLs containing good proteins live inside AFOD and AFCC treated cancer tumor after it has been detached completely from the body of the nude mice #3-7, and when cancer tumor is cultured, the GOOD HEALTHY CELLs of AFOD and AFCC continue to live.

121. The method of claim 1, wherein the GOOD HEALTHY CELLs containing good proteins can help grow hair on the nude mice head after the mice with breast cancer has been treated with products AFOD and AFCC as observed on mice #4-6.

122. The method of claim 1, wherein the GOOD HEALTHY CELLs containing good proteins can help restore the immune system on the nude mice with limited to no immune system after treated with products AFOD and AFCC.

123. The shelf life of protein products from plasma or from other source can be longer than the existing indications as the GOOD HEALTHY CELLs containing good proteins are living therefore the efficacy of the GOOD HEALTHY CELLs containing good protein can be effective up to 10 years like in human albumin and immunoglobulin.

124. The process of making the medium derived from any source to harvest any cell—named KH cells—KH cells are good healthy cells in which the RNA synthesizes good proteins that send signal to the DAMAGED, SICK, AND BAD CELLS that triggers that synthesis of good proteins that transform these cells to become GOOD healthy cells.

125. The process of making the medium derived from any source to harvest any cell—named KH cells—KH cells are good healthy cells in which the RNA synthesizes good proteins that send signal to the other currently undamaged cells to synthesis of good proteins to protect them from being DAMAGED, INFECTED and PRONE to DNA and other cellular alterations.

126. The process of making the medium derived from any source to harvest any cell—named KH cells—KH cells are good healthy cells in which the RNA synthesizes good proteins that send signal to the body to produce new cells that are healthy and forbid them from being affected by intra- and extracellular damaging signals.

127. The process of making the medium derived from any source to harvest any cell—named KH cells—KH cells are good healthy cells in which the RNA synthesizes good proteins to increase the protein yield for the application of the cell expression of human healthcare, animal healthcare and plant healthcare including fertilizer and maximize production of medicine, food, fruit, juice, meat, seafood and plants.

128. The KH cells which are the good healthy cells from human, animal, plant or from any other sources NEVER DIE.

129. The KH cells which are the good healthy cells from any source can survive virus inaction methods, such as solvent detergent technology, dry heating up to 120 degrees Celsius for one and half hours, pasteurization, double pasteurization, nano filtration and 40% alcohol addition to it.

130. A medium selected from the group consisting of KH101 medium consisting of non-sticky rice cells, KH103 medium consisting of soy bean cells, KH104 medium consisting of Orange cells, KH105 medium consisting of Grape cells backspace, KH106 medium consisting of Apple, KH107 medium consisting of sticky rice cells, KH109 medium consisting of white wine cells, KH110 medium consisting of red wine cells, KH111 medium consisting of green bean cells, KH112 medium consisting of Oat cells, KH113 medium consisting of Chestnut cells, KH114 medium consisting of Dorian cells, KH115 medium consisting of Raspberry cells, KH116 medium consisting of Pear cells, KH117 medium consisting of Jack Fruit cells, KH118 medium consisting of Water Apple cells, KH119 medium consisting of Mangosteen cells, KH120 medium consisting of Lettuce cells, KH121 medium consisting of Corn cells, KH122 medium consisting of Sweet Potato cells, KH123 medium consisting of Cucumber cells, KH124 medium consisting of Tomato cells, KH125 medium consisting of Dragon Fruit cells, KH126 medium consisting of Water Melon cells, KH127 medium consisting of Lychee cells, KH128 medium consisting of Yellow melon cells, KH129 medium consisting of Pineapple cells, KH130 medium consisting of Coconut cells, KH131 medium consisting of Mint cells, KH132 medium consisting of Hot Pepper cells, KH133 medium consisting of Black Pepper cells, KH134 medium consisting of Carrot cells, in which the RNA synthesizes good proteins that. 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals to increase the protein yield for the application of the cell expression of human healthcare, animal healthcare and plant healthcare including fertilizer and maximize production of medicine, food, fruit, juice, meat, seafood and plants.

131. The medium of claim 130, wherein the medium is KH101 medium consisting of non-sticky rice cells.

132. The medium of claim 130, wherein the medium is KH103 medium consisting of soy bean cells.

133. The medium of claim 130, wherein the medium is KH104 medium consisting of Orange cells.

134. The medium of claim 130, wherein the medium is KH105 medium consisting of Grape.

135. The medium of claim 130, wherein the medium is KH106 medium consisting of Apple cells.

136. The medium of claim 130, wherein the medium is KH107 medium consisting of sticky rice.

137. The medium of claim 130, wherein the medium is KH109 medium consisting of white wine cells.

138. The medium of claim 130, wherein the medium is KH110 medium consisting of red wine cells.

139. The medium of claim 130, wherein the medium is KH111 medium consisting of green bean cells.

140. The medium of claim 130, wherein the medium is KH112 medium consisting of Oat cells.

141. The medium of claim 130, wherein the medium is KH113 medium consisting of Chestnut cells.

142. The medium of claim 130, wherein the medium is KH114 medium consisting of Dorian cells.

143. The medium of claim 130, wherein the medium is KH115 medium consisting of Raspberry cells.

144. The medium of claim 130, wherein the medium is KH116 medium consisting of Pear cells.

145. The medium of claim 130, wherein the medium is KH117 medium consisting of Jack Fruit cells.

146. The medium of claim 130, wherein the medium is KH118 medium consisting of Water Apple cells.

147. The medium of claim 130, wherein the medium is KH119 medium consisting of Mangosteen cells.

148. The medium of claim 130, wherein the medium is KH120 medium consisting of Lettuce cells.

149. The medium of claim 130, wherein the medium is KH121 medium consisting of Corn cells.

150. The medium of claim 130, wherein the medium is KH122 medium consisting of Sweet Potato cells.

151. The medium of claim 130, wherein the medium is KH123 medium consisting of Cucumber cells.

152. The medium of claim 130, wherein the medium is KH124 medium consisting of Tomato cells.

153. The medium of claim 130, wherein the medium is KH125 medium consisting of Dragon Fruit cells.

154. The medium of claim 130, wherein the medium is KH126 medium consisting of Water Melon cells.

155. The medium of claim 130, wherein the medium is KH127 medium consisting of Lychee cells.

156. The medium of claim 130, wherein the medium is KH128 medium consisting of Yellow melon cells.

157. The medium of claim 130, wherein the medium is KH129 medium consisting of Pineapple cells.

158. The medium of claim 130, wherein the medium is KH130 medium consisting of Coconut cells.

159. The medium of claim 130, wherein the medium is KH131 medium consisting of Mint cells.

160. The medium of claim 130, wherein the medium is KH132 medium consisting of Hot Pepper cells.

161. The medium of claim 130, wherein the medium is KH133 medium consisting of Black Pepper cells.

162. The medium of claim 130, wherein the medium is KH134 medium consisting of Carrot cells.

163. The medium of claim 130, wherein the medium is a combination of at least one of the KH mediums that produces a meal with a total unit of good healthy proteins.

164. The medium of claim 130, wherein the medium is a combination of at least one of the KH mediums that produces a drink with a total unit of good healthy proteins.

165. The medium of claim 130, wherein the medium is KH201 medium consisting of Green Mussel cells.

166. The medium of claim 130, wherein the medium is KH202 medium consisting of Duck cells.

167. The medium of claim 130, wherein the medium is KH203 medium consisting of Giant clam cells.

168. The medium of claim 130, wherein the medium is KH204 medium consisting of Alaskan Crab cells.

169. The medium of claim 130, wherein the medium is KH205 medium consisting of Pork cells.

170. The medium of claim 130, wherein the medium is KH206 medium consisting of Beef cells.

171. The medium of claim 130, wherein the medium is KH207 medium consisting of Mackerel Fish cells.

172. The medium of claim 130, wherein the medium is KH208 medium consisting of Chicken cells.

173. The medium of claim 130, wherein the medium is KH209 medium consisting of Shrimp cells.

174. The medium of claim 130, wherein the medium is KH210 medium consisting of Egg Yolk cells.

175. The medium of claim 130, wherein the medium is KH211 medium consisting of Egg White cells.

176. The medium of claim 130, wherein the medium is KH212 medium consisting of Shanghai Crab cells.

177. The medium of claim 130, wherein the medium is KH213 medium consisting of Crawfish cells.

178. The medium of claim 130, wherein the medium is KH214 medium consisting of Salmon Fish cells.

179. The medium of claim 130, wherein the medium is KH301 medium consisting of Chinese Yam cells.

180. The medium of claim 130, wherein the medium is KH302 medium consisting of Chinese worm medicine (Dong Chong Xia Cao) cells.

181. The medium of claim 130, wherein the medium is KH303 medium consisting of Tibet leaves cells.

182. The medium of claim 130, wherein the medium is KH304 medium consisting of Bovine Milk for newly born baby cells.

183. The medium of claim 130, wherein the medium is KH305 medium consisting of Bovine Milk for three month old babies cells.

184. The medium of claim 130, wherein the medium is KH306 medium consisting of Bovine Milk for six month old babies cells.

185. The medium of claim 130, wherein the medium is KH307 medium consisting of Bovine Milk for one year old baby cells.

186. The medium of claim 130, wherein the medium is KH308 medium consisting of Bovine Milk cells.

187. The medium of claim 130, wherein the medium is KH309 medium consisting of Human Placenta cells.

188. The medium of claim 130, wherein the medium is KH135 medium consisting of banana cells.

189. The medium of claim 130, wherein the medium is KH136 medium consisting of big banana cells.

190. The medium of claim 130, wherein the medium is KH137 medium consisting of small banana cells.

191. The medium of claim 130, wherein the medium is KH138 medium consisting of star fruit cells.

192. The medium of claim 130, wherein the medium is KH139 medium consisting of pomegranate cells.

193. The medium of claim 130, wherein the medium is KH140 medium consisting of plum cells.

194. The medium of claim 130, wherein the medium is KH141 medium consisting of mango cells.

195. The medium of claim 130, wherein the medium is KH142 medium consisting of green hot pepper cells.

196. The medium of claim 130, wherein the medium is KH143 medium consisting of red sweet pepper cells.

197. The medium of claim 130, wherein the medium is KH144 medium consisting of green sweet pepper cells.

198. The medium of claim 130, wherein the medium is KH145 medium consisting of daisy flower cells.

199. The medium of claim 130, wherein the medium is KH146 medium consisting of puer tea cells.

200. The medium of claim 130, wherein the medium is KH147 medium consisting of walnut cells.

201. The medium of claim 130, wherein the medium is KH148 medium consisting of white bread cells.

202. The medium of claim 130, wherein the medium is KH149 medium consisting of brown bread cells.

203. Any combination of any substance that contains good KH healthy cells in which the RNA synthesizes good proteins that. 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals to increase the protein yield for the application of the cell expression of human healthcare, animal healthcare and plant healthcare including fertilizer and maximize production of medicine, food, fruit, juice, meat, seafood and plants.

204. A good protein in which the KH good healthy cell whose RNA exists. 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals.

205. A bad, damaged or sick cell whose RNA synthesize a bad protein that 1—Send signal to the good healthy CELLS that triggers the synthesis of bad proteins that transform these cells to become bad, damaged or sick cells. 2—Send signal to the other currently undamaged cells to synthesis a bad protein to become DAMAGED, INFECTED and PRONE to DNA and other cellular alterations. 3—Send signal to the body to produce new cells that are unhealthy and are help them to be affected by intra- and extracellular damaging signals to cause the deficiency inhibitor disease and cancers.

206. Any genetic modification of any human being, animal, plant or living organism will 1—Send signal to the good healthy CELLS that triggers the synthesis of bad proteins that transform these cells to become bad, damaged or sick cells. 2—Send signal to the other currently undamaged cells to synthesis a bad protein to become DAMAGED, INFECTED and PRONE to DNA and other cellular alterations. 3—Send signal to the body to produce new cells that are unhealthy and are help them to be affected by intra- and extracellular damaging signals to cause the deficiency inhibitor disease, cancers and DEATH (gene therapy).

207. Any living organism including human, animal or plant whose RNA synthesize a bad protein can be on selected diet which contain a good KH healthy cell to 1—Send signal to the DAMAGED, SICK, AND BAD CELLS that triggers that synthesis of good proteins that transform these cells to become GOOD healthy cells. 2—Send signal to the other currently undamaged cells to synthesis of good proteins to protect them from being DAMAGED, INFECTED and PRONE to DNA and other cellular alterations. 3—Send signal to the body to produce new cells that are healthy and forbid them from being affected by intra- and extracellular damaging signals to slow the progression of the disease or cancer and recover.
